(19) United States
(12) Patent Application Publication
(10) Pub. No.: US 2022/0084664 A1
(43) Pub. Date: Mar. 17, 2022

(54) DYNAMIC HEALTH RECORDS

(71) Applicant: Leonard H. Ginsburg, Merion, PA (US)

(72) Inventor: Leonard H. Ginsburg, Merion, PA (US)

(21) Appl. No.: 17/187,843

(22) Filed: Feb. 28, 2021

Related U.S. Application Data
(63) Continuation-in-part of application No. 17/008,586, filed on Aug. 31, 2020.
(60) Provisional application No. 62/893,688, filed on Aug. 29, 2019.
(60) Provisional application No. 62/907,410, filed on Sep. 27, 2019.
(60) Provisional application No. 62/983,350, filed on Feb. 28, 2020.
(60) Provisional application No. 62/987,165, filed on Mar. 9, 2020.
(60) Provisional application No. 63/026,547, filed on May 18, 2020.
(60) Provisional application No. 63/116,684, filed on Nov. 20, 2020.
(60) Provisional application No. 63/127,840, filed on Dec. 18, 2020.

(51) Int. Cl. G16H 40/20; G16H 15/00

(52) U.S. Cl. CPC G16H 40/20; G06F 3/0481; G16H 15/00

(57) ABSTRACT

A computer implemented method of creating medical orders includes generating a dashboard display comprising one or multiple visible panels having data corresponding to different respective medical services, receiving a request to create an order in response to user interaction with a first one of the multiple panels, retrieving first medical information as a function of information associated with the panel from which the request was received, and populating a place order panel with the retrieved first medical information.

### BACKGROUND

Caregivers are often called upon to make rapid life and death decisions based on a patient's condition in the context of a medical history as presented, for example, in an Electronic Medical Record (“EMR”). However, the visual display systems for EMR's are difficult to understand and require the user to move through multiple screens, interfaces, and menus to obtain the disparate information needed to make a medical decision. This creates great difficulties when caring for multiple patients in a busy practice and is compounded when different doctors provide care for the same patient. Moreover, the complex interfaces associated with EMRs are particularly problematic at the point of care as they slow caregivers down and distract them from meaningful face-time, caring for patients. Communication and sharing care for a particular patient between multiple health care providers has become more difficult. Now, rather than a fax or short dictated medical summary, caregivers are now sending voluminous amounts of information as the EMR has no organized way to correlate associated data over time nor share information across different EMR's, and non-integrated systems.

Furthermore, no system displays clinical and examination findings, medications actually taken by the patient, procedures, and diagnostic tests in a way that a user can discover at a glance if treatment is effective. No system, provides the ability for the user to visualize in context to allow them to double check that the orders and medications they are placing, are in fact the exact correct ones they intend to order. There is no system that displays, correlates, and highlights interrelated data points that can make a difference in the life of each patient. When information is displayed in a flow chart in an EMR, the presentations are not able to be adjusted manually and dynamically for an individual patient. Now that former paper medical records have been digitalized, data is very difficult to find. Medical care and the associated data dispersed in the computer has become so complex that what is needed is the type of intelligent, actionable visual display system that in context automatically adjusts the presentation, sorts, compresses and highlights the data the user needs to be able to make a medical decision and visualize the cause-and-effect of treatment.

In health care, EMR systems, and practice and image management systems borrow dashboards and displays from other fields. These displays often have time or date in one direction, with width or height consistent and variable factors that are being shown or measured in the other direction. The importance of certain occurrences in time may deserve more or less emphasis. Time spent does not always equate with work effort or results.

While these traditional methods of evaluating data may work for some fields, these displays are sorely lacking in the medical field, which demands an entire new approach to all existing displays, flow charts, spreadsheets and dashboards. In medicine a particular date of service could have an occurrence with much greater significance than another date. One date might be a routine office examination and another cancer discovered. An encounter with one provider, can be much more impactful than another. Both dates of service may have common features such as a particular clinical measurement. But, what is needed is a way to express the intensely different occurrences, so that at a glance with limited space and time, the critical information for a particular patient is conveyed. Unfortunately, EMR's, if they have a flow chart, simply display data in similar ways used outside of medicine. Existing spreadsheets such as excel, may work for assisting accountants or even building an airplane. Once one plane is built, the next can be built the exact same way. Replicating even one human being has never been accomplished and no two people are the same nor react to medical care the exact same way.

What is needed is an entirely new display approach to presenting, organizing and measuring data tailored for the human being. A simple, elegant solution that enables caregivers to synthesize information and populate and document a chart and even display orders as created when seeing a patient. A single presentation that enables a caregiver to identify medical problems through data visualization, where data is presented and displayed in an intuitive, easy to view manner.

### SUMMARY

The above and other needs in the art are addressed by a data command center visual display system and associated methods for displaying data on a display from multiple data sources and allowing navigation amongst the data without leaving the display of the visual display system. Numerous technical issues rooted in computer technology must be solved for the data to be presented to the visual display system so that the data may be displayed in the command center using a single display interface. For example, the visual display system must provide access to the requisite health information systems and third-party support services whereby the data may be accessed, processed, and presented without unacceptable delay. Also, the display data must be collected and ordered to facilitate the various combinations of the data into respective display panels that may be navigated on the interface. For example, it is desirable for the data to be configured in a task-based or specialty-specific display configuration for use by physicians, for example. To do this, various features in prior art systems needed to be acquired and combined in a new way to facilitate access to the features without having to navigate away from the display. For example, conventional EMR systems provide interfaces to third party prescription ordering systems but require the user the navigate to another system and away from the EMR interface. Accessing ordering panels without leaving the display becomes particularly difficult where the display space is limited as is the case for many physicians who use portable display devices and mobile computers. The structural embodiments described herein address these technical issues to generate the command center visual display system embodiments described herein.

In exemplary embodiments, such a data command center visual display system in accordance with the present principles includes a patient database that stores patient identification information, patient insurance information, patient medical history information, a computer readable storage medium having stored thereon instructions thereon, and a processor that executes the instructions to perform operations including creating a plurality of adjustable display panels configured to display predetermined combinations of the patient identification information, patient insurance information, patient medical history information, and creating a patient flowsheet that integrates the patient medical history information into a table that presents the patient's medical history by visit to at least one physician with respective procedures or actions performed during each visit represented as first icons identifying the procedure or action performed and second icons enabling selection of a new procedure or action, where the first and second icons provide links to associated patient medical information and ordering display panels that may be accessed without leaving the display. In response to selection of the second icon by a user of the visual display system, an ordering display panel is presented to the display in addition to the adjustable display panels and patient flowsheet. The desired procedures or actions may be ordered from the ordering display panels while relevant portions of the patient's medical history are still visible on the display screen. The scope of the claims also contemplates corresponding methods performed by the visual display system and users thereof.

In exemplary embodiments, the ordering display panel comprises an ePrescribing panel for ordering medication or a medical procedure ordering panel for ordering a medical procedure. By way of example, the medical procedure ordering panel for ordering a medical procedure may further provide a link to the quality reporting panel that displays quality reporting metrics and/or peer data related to the procedure that is being ordered. All of such ordering display panels are configured in the context of the display to conserve display space so that the ordering display panel may be displayed while still being able to view the medical history data, for example.

In other exemplary embodiments, the ordering display panel comprises an imaging order panel for ordering a medical image of the patient or a lab order panel for ordering a lab test of the patient. In still other embodiments, instructions are provided that when executed create an image icon in an adjustable display panel and/or the patient flowsheet that, when selected by the user of the visual data system, opens a display window for viewing of one or more images without leaving the display.

In other exemplary embodiments, the visual display system incorporates financial data with the patient medical history data into the display panels. Such a visual display system includes a patient database that stores patient identification information, patient insurance information, patient medical history information, and patient payment information, a computer readable storage medium having stores thereon instructions thereon, and a processor that executes the instructions to perform operations including creating a plurality of adjustable display panels configured to display predetermined combinations of the patient identification information, patient insurance information, patient medical history information, and patient payment information, and creating a patient flowsheet that integrates the patient medical history information and patient payment information into a table that presents the patient's medical history by visit to at least one physician with respective procedures or actions performed during each visit represented as first icons identifying the procedure or action performed and second icons indicating whether the procedure or action has been paid for in part or in full, the first and second icons providing links to associated patient medical history information and/or patient payment information. In response to selection by a user of the visual display system, the adjustable display panels and patient flowsheet are moved into a task-based or specialty-specific display configuration such that the patient identification information, patient insurance information, patient medical history information, and patient payment information may be accessed without leaving the display. The task-based or specialty-specific display configuration is then presented to the display. In exemplary embodiments, selection of the first icons or second icons open display windows to associated medical history data and/or financial data and overlay a portion of the display with the display windows whereby the associated medical history data and/or financial data may be viewed by the user of the visual display system while the adjustable display panels and the patient flowsheet are displayed in a background on the display. Throughout this description, it will be appreciated that all financial data in the system, including costs to patient, is compartmentalized such that no user may see financial details for users or organizations not authorized in accordance with applicable policies and law. Also, the scope of the claims also contemplates corresponding methods performed by the visual display system and users thereof.

The visual display system includes a number of features that enable accessing information on the display. For example, third icons are provided in the patient flowsheet or display panels that include links to compliance information about compliance with insurance guidelines and/or good clinical practice guidelines for a procedure or action associated with each third icon. In exemplary embodiments, the compliance information includes aggregated medical treatment guidelines and an overview outlining similarities and differences amongst different medical treatment guidelines making up the aggregated medical treatment guidelines. The aggregated medical treatment guidelines may include information related to recommended follow-up with the patient, information related to procedures permitted or prevented by the patient's insurance or contra-indications, and information relating to proper billing for the procedure or action associated with a third icon selected from the patient flowsheet or display panels. In exemplary embodiments, the visual display system provides access to a clinical decision support system that uses a rules engine and/or natural language processing to aggregate the medical treatment guidelines and to generate the overview outlining similarities and differences amongst different medical treatment guidelines making up the aggregated medical treatment guidelines. The clinical decision support system and/or natural language processing system may further compare medical data to notice patterns, errors and anomalies in different entries or notes, find discrepancies in payments, alert the user of the visual display system about inconsistent medical documentation or improper orders, speed up the process of complying with regulations, alert the user of the visual display system that a plan or order is inconsistent with a preferred practice plan for a patient, or warn the user of the visual display system that billing certain procedures might not be covered. The natural language processing system may also be accessed parse notes in the patient flowsheet or display panels for potential ICD10 codes or alternative diagnosis.

The visual display system also includes a display configuration that enables users of the visual display system to order medications, diagnostic tests, images, procedures, and the like directly from the patient flowsheet or display panel. For example, an icon or link in the patient flowsheet or display panel may include an ePrescribing panel for ordering medication or a medical procedure ordering panel for ordering a medical procedure. The medical procedure ordering panel may further include a link to a quality reporting panel that displays quality reporting metrics and/or peer data related to the procedure that is being ordered. In other embodiments, an icon or link in the patient flowsheet or display panel may include an imaging order panel for ordering a medical image of the patient or a lab order panel for ordering a lab test of the patient. In still other embodiments, an image icon is provided in an adjustable display panel and/or the patient flowsheet that, when selected by the user of the visual data system, opens a display window for viewing of one or more images without leaving the display screen. In other embodiments, an alert icon is provided in an adjustable display panel and/or the patient flowsheet that, when selected by the user of the visual data system, opens an alert message without leaving the display. In still other embodiments, one of the display panels may be configured to accept today's visit notes from the user of the visual display system in connection with a patient visit for storage for access with other data of the one display panel.

Other novel features in exemplary embodiments include a moveable note icon for association with context information in a corresponding one of the adjustable display panels and/or the patient flowsheet. The note icon moves with the context information as the context information is moved on the display. When the note icon is selected, the user of the visual display system may enter a note relating to the context information.

In still other embodiments, data input by the user of the visual display system may trigger auto-population of information in the adjustable display panels and patient flowsheet and auto-population of the patient's medical record in an electronic medical record system. In the exemplary embodiments, the auto-population occurs without the user of the video display system leaving the display.

In other embodiments, new clinical information for the patient is provided to a diagnosis evaluation algorithm for comparison of the new clinical information with previous corresponding clinical information for the patient to determine whether the new clinical information is indicative of an improvement or worsening of the patient's medical condition. The visual display system further generates diagnosis indicators providing a visual representation of an improvement of a medical problem, disease, or symptom, or a worsening of a medical problem, disease, or symptom as a result of taking a particular medication or undergoing a particular medical procedure and displays the diagnosis indicators in the adjustable display panels and/or the patient flowsheet.

Other embodiments of the visual display system allow for increased speed of data presentation by a local database that stores a subset of patient identification information, patient insurance information, patient medical history information, and patient payment information, where the subset includes the patient identification information, patient insurance information, patient medical history information, and patient payment information for patients having an appointment within a predetermined time window.

The visual display system in exemplary embodiments includes interfaces to an external health information system and third party service systems. In exemplary embodiments, the external health information system includes at least one of an electronic medical records system, a practice management system, a health information exchange, a picture archive and communications system, a clearing house/billing system, and a laboratory system. On the other hand, the third party service systems may include one or more of an ePrescribing system, an insurance verification/referral/pre-authorization system, a system for establishing medical necessity by verifying that a procedure or medication is associated with a correct ICD10 code supporting its use, a clinical services pricing and location system, a claim status checking system, services in support of the National Correct Coding Initiative, services to proactively ensure claims are coded correctly to prevent issues in billing, claims compliance services that evaluate claims against National Coverage Determination (NCD) and Local Coverage Determination (LCD) guidelines as well as local insurance regulations to establish and document medical necessity, a natural language processing system, and artificial intelligence/cognitive systems that provide clinical decision support.

In exemplary embodiments, the patient identification information, patient insurance information, patient medical history information, and patient payment information is stored in the patient database in transactional tables that capture clinical and billing data and reporting tables where data is aggregated for a particular physician, practice, health system or other entity. Each table uses a surrogate primary key that is a unique value within the table used to identify a row that is not directly tied to data in that row. In the exemplary embodiments, XML code moves and stores different display panel and flowsheet views. The XML code further identifies a collection of panels and tabs, wherein within each panel is a panel ID that links the panel to a tab, the panel's position, and whether or not the panel is stacked with another panel. The XML code may also set up the display panels and patient flowsheet on the display by, for example, identifying a collection of columns and, for each column, a name of the column along with a data source. The display panels so configured are presented to the display for selection and display panel frames on the display screen are manipulated for receiving selected display panels.

In other exemplary embodiments, the patient flowsheet is organized around patient medical information corresponding to a particular disease state and/or procedures and/or insurance coverage and/or actions for treating the particular disease state.

The patient database may also be adapted to include patient medical history information from a plurality of medical care providers whereby the patient flowsheet may be adapted to include medical history information from more than one medical care provider in order to provide shared treatment of the patient in the patient flowsheet. In other embodiments, a summary table may be provided that illustrates everything the user of the visual display system has done for each patient in a particular time frame or for each patient having a particular disease state in a particular time frame. The summary table may also include information from other medical care providers who are providing shared treatment of the patient. If financial data, cost, charge, payment is on the summary table with the medical data, this data is compartmentalized such that no user may see financial details for users or organizations not authorized in accordance with applicable policies and law.

In yet other embodiments, a data command center visual display system is provided that presents dynamic data to a display. The command center visual display system includes a plurality of adjustable display panels configured to display predetermined combinations of patient identification information and patient medical information. A patient flowsheet is created that includes a table that presents the patient's medical information by medical service, medical procedure, diagnostic test, medication, and diagnosis that is prescribed, ordered, performed, or selected during respective encounters with at least one medical care provider. In response to selection by a user, at least two adjustable display panels containing medical information relating to one or more patients in the patient flowsheet are presented to the display in a single view. The user may edit or move the medical information or the patient identification information within the display panels while the display panels are simultaneously open.

In some embodiments, a method for rules-based data display in a data command center including a medical records dashboard including one or more windows including information received or derived from at least one patient database, the medical records dashboard comprising a display, using the one or more windows, of at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the display according to at least one of a time and a date that the medical services, the clinical data, the examination findings, the diagnostic tests and the procedures were performed on the one or more patients, the method includes receiving patient-related data from the at least one patient database, comparing the received patient-related data with configuration rules to determine which portions of the received patient-related data are to be displayed in data entry fields of the medical records dashboard, identifying collapsible data entry fields of the at least one collapsible data entry field of the medical records dashboard that are determined to not have any patient-related data to display as collapsed data entry fields, displaying patient-related data in the data entry fields of the medical records dashboard in accordance with the configuration rules and collapsing data entry fields of the medical records dashboard identified as collapsed data entry fields.

In some embodiments, a data command center visual display system that displays data on a display includes a computing device comprising at least one processor, a non-transitory computer-readable medium, having stored thereon, software instructions that when executed by the at least one processor of the computing device, cause the computing device to perform operations comprising at least, linking to and receiving patient related medical records including patient data from at least one patient data source, and displaying a medical records dashboard including one or more windows, the medical record dashboard capable of displaying, using the one or more windows, patient data from at least one patient data source including at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the one or more windows according to at least one of a time and a date that the medical services, the clinical data, the examination findings, the diagnostic tests and the procedures were performed on the one or more patients, wherein a display of patient data in the medical records dashboard is determined by: comparing the patient data with configuration rules to determine which portions of the patient data are to be displayed in the data entry fields of the medical records dashboard, identifying collapsible data entry fields of the at least one collapsible data entry field of the medical records dashboard that are determined to not have patient data to display as collapsed data entry fields, and displaying patient data in the data entry fields of the medical records dashboard in accordance with the configuration rules and collapsing data entry fields of the medical records dashboard identified as collapsed data entry fields.

In some embodiments, a method for unique patient identification of a subject patient in a data command center including patient-related data received or derived from at least one patient database includes collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning phase.

In some embodiments, a data command center visual display system for determining a unique patient identification includes a computing device comprising at least one processor, a non-transitory computer-readable medium, having stored thereon, software instructions that when executed by the at least one processor of the computing device, cause the computing device to perform operations comprising at least: linking to and receiving patient related medical records including patient data from at least one patient data source, collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning.

In some embodiments, a method for medication management and display in a data command center comprising one or more windows for display and including information received or derived from at least one patient database, the data command center displaying, using the one or more windows, at least one of medical services, clinical data, examination findings, diagnostic tests, and procedures performed on one or more patients, the one or more windows comprising a plurality of data entry fields for displaying the information received or derived from the at least one patient database, wherein the at least one of the medical services, the clinical data, the examination findings, the diagnostic tests, and the procedures are arranged in on the one or more windows according to at least one of a time and a date that the medical services, the clinical data, the examination findings, the diagnostic tests and the procedures were performed on the one or more patients, includes determining, from at least one of the information received or derived from the at least one patient database and the at least one of the medical services, the clinical data, the examination findings, the diagnostic tests, and the procedures, medications administered to the one or more patients, generating a respective graphical representation for each of the determined medications administered to the one or more patients, and displaying at least one generated, respective graphical representation of at least one medication administered to a patient in the at least one or more windows in context with at least one of the information received or derived from the at least one patient database and the at least one of the medical services, the clinical data, the examination findings, the diagnostic tests, and the procedures, wherein the at least one generated, respective graphical representation of the at least one medication administered to the patient is arranged in on the one or more windows according to at least one of the times and the dates that the at least one medication was being administered to the patient.

In some embodiments, a data command center visual display system that displays data on a display includes a computing device comprising at least one processor, a non-transitory computer-readable medium, having stored thereon, software instructions that when executed by the at least one processor of the computing device, cause the computing device to perform operations including at least, linking to and receiving patient related medical records including patient data from at least one patient data source, wherein the patient data includes at least one of medical services, clinical data, examination findings, diagnostic tests, and procedures performed on one or more patients, determining, from at least one of the patient data and the at least one of the medical services, the clinical data, the examination findings, the diagnostic tests, and the procedures, medications administered to the one or more patients, generating a respective graphical representation for each of the determined medications administered to the one or more patients, and displaying using the one or more windows, at least one of medical services, clinical data, examination findings, diagnostic tests, and procedures performed on one or more patients and at least one generated, respective graphical representation of at least one medication administered to a patient in context with at least one of the patient data and the at least one of the medical services, the clinical data, the examination findings, the diagnostic tests, and the procedures, wherein the at least one of the medical services, the clinical data, the examination findings, the diagnostic tests, and the procedures are arranged on the display according to at least one of a time and a date that the medical services, the clinical data, the examination findings, the diagnostic tests and the procedures were performed on the one or more patients, and wherein the at least one generated, respective graphical representation of the at least one medication administered to the patient is arranged on the display according to at least one of the times and the dates that the at least one medication was being administered to the patient.

In some embodiments, a method for a display of a graphical representation of complete medical history of a patient in a data command center comprising one or more windows for display and including patient-related data received or derived from at least one patient database, the method includes determining, from the patient-related data, a complete medical history of at least one patient including at least one of medical services, clinical data, examination findings, diagnostic tests, medications administered to and procedures performed on a patient, generating a graphical representation of the determined complete medical history of the patient including the at least one of medical services, clinical data, examination findings, diagnostic tests, medications administered to and procedures performed on the patient, and displaying the generated graphical representation in the at least one or more windows according to at least one of a time and a date that the at least one of the medical services, the clinical data, the examination findings, the diagnostic tests, and the procedures the medical services, the clinical data, the examination findings, the diagnostic tests and the procedures were performed on the one or more patients and at least one of the times and the dates that the medications were being administered to the patient, wherein a user is enabled to select a location in the displayed graphical representation and details regarding the at least one of medical services, clinical data, examination findings, diagnostic tests, medications administered to and procedures performed on the patient related to that selected location are presented to the user.

In some embodiments, a method for in-context display of images and patient related information includes retrieving patient related information from at least one patient database or server, displaying at least one medical record dashboard including one or more windows for displaying, using a single display interface, patient related information retrieved from or derived from the at least one patient database or server including at least one of medical services, clinical data, examination findings, diagnostic tests, and procedures performed on one or more patients. In some embodiments, the one or more windows include a plurality of data entry fields for displaying the patient related information received or derived from the at least one patient database, wherein the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. In such embodiments, the method can further include generating at least one visual representation of at least one image related to the at least one of the medical services, the clinical data, the examination findings, the diagnostic tests, and the procedures performed on the one or more patients, and displaying at least one of the at least one generated visual representations on the display in at least one of the plurality of data entry fields, such that when a displayed visual representation is selected, a respective image is displayed concurrently with the patient related information on the display.

In some embodiments, a visual representation of a patient-related image in accordance with the present principles can include at least one thumbnail representation of an image.

In some embodiments, a user of an in-context image management system in accordance with the present principles is able to select an image to view by reviewing the thumbnail representations of the available patient-related images.

In some embodiments, a selection of a displayed visual representation of patient-related images can include clicking on the displayed visual representation of patient-related images using a pointing device.

In some embodiments, a selection of a displayed visual representation of patient-related images can include hovering over a displayed visual representation of patient-related images using a pointing device.

In some embodiment of the present principles, a system for in-context display of images and patient related information include a computing device comprising at least one processor and a non-transitory computer-readable medium, having stored thereon, software instructions. In such embodiments, when the software instructions are executed by the at least one processor of the computing device, the system is configured to perform operations including at least retrieving patient related information from at least one patient database or server, displaying at least one medical record dashboard including one or more windows for displaying, using a single display interface, patient related information retrieved from or derived from the at least one patient database or server including at least one of medical services, clinical data, examination findings, diagnostic tests, and procedures performed on one or more patients. In such embodiments, the one or more windows include a plurality of data entry fields for displaying the patient related information received or derived from the at least one patient database, and where the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. In such embodiments the system is further configured to perform operations including generating at least one visual representation of at least one image related to the at least one of the medical services, the clinical data, the examination findings, the diagnostic tests, and the procedures performed on the one or more patients, and displaying at least one of the at least one generated graphical representation on the display in at least one of the plurality of data entry fields, such that when a displayed graphical representation is selected, a respective image is displayed concurrently with the patient related information on the display.

Other and further embodiments in accordance with the present principles are described below.

### DESCRIPTION OF THE DRAWINGS

FIG. 1 depicts a high-level block diagram of a Data Command Center in accordance with an embodiment of the present principles.

FIG. 2 depicts a high-level block diagram of a computing device 200 suitable for use with embodiments of a Data Command Center in accordance with the present principles.

FIG. 3 depicts a high-level diagram of a medical records dashboard selection window of, for example, a medical record system useful for selecting and launching at least a portion of a Data Command Center (CC) in accordance with an embodiment of the present principles.

FIG. 4A depicts an example of a medical records dashboard of the Data Command Center in accordance with an embodiment of the present principles.

FIG. 4B depicts a portion of the medical records dashboard of FIG. 4A in accordance with some embodiments of the present principles.

FIG. 4C depicts greater detail of at least some portions of the medical records dashboard of FIG. 4A in accordance with some embodiments of the present principles.

FIG. 4D depicts greater detail of at least some portions of the medical records dashboard of FIG. 4A in accordance with some embodiments of the present principles.

FIG. 5A depicts the medical records dashboard including a medical summary update process in accordance with some embodiments of the present principles.

FIG. 5B depicts a portion of the medical records dashboard including a notes update procedure in accordance with an embodiment of the present principles.

FIG. 6 depicts a user record access process of the medical records dashboard in accordance with an embodiment of the present principles.

FIG. 7A depicts an image access window of the medical records dashboard in accordance with an embodiment of the present principles.

FIG. 7B depicts the functionality of three specific rows in the user interface in accordance with an embodiment of the present principles.

FIG. 8 depicts a medical records and diagnosis update process of the medical records dashboard in accordance with an embodiment of the present principles.

FIG. 9 depicts a medical record update marker process of the medical records dashboard in accordance with an embodiment of the present principles.

FIG. 10 depicts a medical record update marker process of the medical records dashboard in accordance with an embodiment of the present principles.

FIG. 11 depicts a portion of a medical records dashboard in accordance with another embodiment of the present principles.

FIG. 12 depicts a portion of the medical records dashboard of FIG. 11 in accordance with an embodiment of the present principles.

FIG. 13 depicts a portion of a medical records dashboard configured for display as a function of disease or patient in accordance with another embodiment of the present principles.

FIG. 14 depicts a portion of a medical records dashboard configured for display as a function of disease of a patient and specifically configured to display data related to patients with diabetes in accordance with another embodiment of the present principles.

FIG. 15 depicts an embodiment of a medical records dashboard which can be displayed following a user's selection of at least one medical records dashboard from the medical records dashboard selection window in accordance with another embodiment of the present principles.

FIG. 16 depicts a ledger window accessible from the medical records dashboard of FIG. 15 in accordance with an embodiment of the present principles.

FIG. 17 depicts an embodiment of a Data Command Center menu including a medical records dashboard implemented as a data interface to a medical record system in accordance with an embodiment of the present principles.

FIG. 18 depicts an embodiment of a User View control panel that can be part of the View/Task menu of the medical records dashboard of the Data Command menu of the embodiment of FIG. 17 in accordance with an embodiment of the present principles.

FIG. 19 depicts an embodiment sticky note panel of the Data Command Center menu of FIG. 17, which is activated when the add sticky notes icon in FIG. 17 is selected in accordance with an embodiment of the present principles.

FIG. 20 depicts an embodiment of a Patient Information Panel of the Data Command Center menu, which can be activated when the Patient Information Bar is selected in accordance with an embodiment of the present principles.

FIG. 21 depicts an embodiment of a Patient Insurance Panel of the Data Command Center menu, which can be activated when the Patient Insurance Bar is selected in accordance with an embodiment of the present principles.

FIG. 22A depicts an embodiment of a Today's Visit Notes tab of the Data Command Center menu in accordance with an embodiment of the present principles.

FIG. 22B depicts an embodiment of a Surgeries tab of a medical records dashboard of a Data Command Center in accordance with an embodiment of the present principles.

FIGS. 23A, 23B, 23C, 23D, 23E, 23F, 23G, 23H, and 23I depict an embodiment of a medical records dashboard in accordance with another embodiment of the present principles.

FIG. 24 depicts an embodiment of a co-managed medical records dashboard in the Data Command Center of the present principles in accordance with one embodiment.

FIGS. 25A and 25B depict a medical records dashboard including an ability to launch a Co-Management process in accordance with an embodiment the present principles.

FIGS. 26A and 26B depict a medical records dashboard including a custom template creation process for co-management in accordance with an embodiment of the present principles.

FIG. 27A depicts a first portion of a workflow diagram of a Co Management process in accordance with an embodiment of the present principles.

FIG. 27B depicts a second portion of the workflow diagram of the Co Management process of FIG. 27A in accordance with an embodiment of the present principles.

FIG. 28 depicts a flow diagram of a method for Co-Management of patient information in a medical records dashboard in accordance with an embodiment of the present principles.

FIG. 29 depicts a flow diagram of a method for Unique Patient Identification in a Data Command Center in accordance with an embodiment of the present principles.

FIGS. 30A, 30B, 30C, 30D, 30E, 30F, 30G, 30H, and 30I depict a first embodiment of a Medication Management chart that can be displayed in at least a portion of a medical records dashboard of the present principles in accordance with one embodiment.

FIG. 31 depicts an embodiment of the control panel #1 of the Medication Management chart of FIGS. 30A, 30B, 30C, 30D, 30E, 30F, 30G, 30H, and 30I in accordance with an embodiment of the present principles.

FIGS. 32A and 32B depict a Medication Management Chart that can be displayed as part of a medical records dashboard or as a stand-alone Medication Management tool in accordance with an embodiment of the present principles.

FIGS. 33A1, 33A2, and 33A3 depict an example of how the Control Panel #1 of FIGS. 30A, 30B, 30C, 30D, 30E, 30F, 30G, 30H, and 30I can be implemented by a user to identify start and stop dates for the various medications taken by a user in accordance with an embodiment of the present principles.

FIGS. 33B1, 33B2, 33B3 and 33B4 depict an embodiment of a Medication Management Chart in which icons can be activated to bring up additional information in accordance with an embodiment of the present principles.

FIGS. 33C1, 33C2, and 33C3 depict another embodiment of a Medication Management Chart in which icons can be activated to bring up additional information in accordance with another embodiment of the present principles.

FIGS. 33D1, 33D2, and 33D3 depict an embodiment of the Medication Management Chart in which intraocular pressure, in addition to being listed by number, is also displayed as a vertical line graph, for example as depicted by element 1 in accordance with an embodiment of the present principles.

FIGS. 33E1, 33E2, and 33E3 depict an embodiment of the Medication Management Chart of FIG. 33D in which the control panel can be used to input a reason that a medication has been started or stopped in accordance with an embodiment of the present principles.

FIGS. 33F1, 33F2, and 33F3 depict an embodiment of the Medication Management Chart of FIG. 33D in which the control panel can be used to correct start and stop dates for a medication in accordance with an embodiment of the present principles.

FIGS. 33G1, 33G2, and 33G3 depict depicts an embodiment of the Medication Management Chart of FIG. 33D in which both corrected start and stop dates for a medication taken by a patient and incorrect start and stop dates for a medication taken by a patient and listed for example by a 3^{rd} party data provider such as an EMR can be displayed simultaneously in accordance with an embodiment of the present principles.

FIGS. 33H1, 33H2, and 33H3 depict depicts an embodiment of the Medication Management Chart of FIG. 33D in which a user is alerted that a medication being taken by a patient has changed, even if medications are being listed by class and the new medication is of the same class as the old medication in accordance with an embodiment of the present principles.

FIGS. 33I1, 33I2, and 33I3 depict an embodiment of the Medication Management Chart of FIGS. 33H1-3 in which a user is able to select a portion of a graph to bring up additional information associated with the graph in accordance with an embodiment of the present principles.

FIG. 34 depicts an illustration of a second embodiment of a Medication Management chart that can be displayed in at least a portion of the medical records dashboard of the present principles in accordance with one embodiment.

FIGS. 35A, 35B, and 35C depict a medical records dashboard including a third embodiment of a Medication Management chart in accordance with an embodiment of the present principles.

FIG. 36 depicts a high-level workflow diagram of an embodiment of Medication Management in a Data Command Center in accordance with an embodiment of the present principles.

FIGS. 37A and 37B depict an exemplary embodiment of a Medications Management chart/tool which does not use rows or columns in accordance with an alternate embodiment of the present principles.

FIGS. 38A1, 38A2, and 38A3 depict a first enlarged portion of the Data Command Center.

FIGS. 38B1 and 38B2 depict a second enlarged portion of the Data Command Center.

FIGS. 38C1 and 38C2 depict a third enlarged portion of the Data Command Center.

FIG. 38D depicts a fourth enlarged portion of the Data Command Center.

FIGS. 39A, 39B, and 39C depict an embodiment of a medical records dashboard of a Data Command Center in which a user/medical care provider is enabled to place orders in context with other relevant patient data/information, so as to enable the user/medical care provider to see the future orders in context in an embodiment not using rows and columns in accordance with the present principles.

FIG. 40A depicts a first portion of a workflow diagram of a process for intelligently expanding, collapsing, highlighting or graying out, displaying, and/or hiding columns, rows and/or any other portion of the medical records dashboard in accordance with an embodiment of the present principles.

FIG. 40B depicts a second portion of a workflow diagram of a process for intelligently expanding, collapsing, highlighting or graying out, displaying, and/or hiding columns, rows and/or any other portion of the medical records dashboard in accordance with an embodiment of the present principles.

FIG. 41 depicts a flow diagram of a method for rules-based data display in a data command center comprising a medical records dashboard in accordance with an embodiment of the present principles.

FIG. 42A, 42b, AND 42C depict a graphical view of the entire medical history of a patient as a Whole Life tool in accordance with an embodiment of the present principles.

FIGS. 43A and 43B depict a post appointment summary chart of a Medical Guidance tool in accordance with an embodiment of the present principles.

FIG. 44 depicts an Evaluative Clinical Reporting (ECR) interface in accordance with an embodiment of the present principles.

FIG. 45A depicts a reporting architecture of a Data Command Center in accordance with an embodiment of the present principles.

FIG. 45B depicts an embodiment of the Orchestrator Master Pipeline of FIG. 45A in with an embodiment of the present principles.

FIG. 46 depicts a sequence diagram for executing a report in accordance with an embodiment of the present principles.

FIG. 47 depicts an embodiment of a medical records dashboard of a Data Command Center in which alerts and tasks can be in accordance with an embodiment of the present principles.

FIG. 48 depicts a menu for pre-configuring alerts in accordance with an embodiment the present principles.

FIG. 49 depicts an embodiment of a medical records dashboard in which alerts are configured based on medication in accordance with the present principles.

FIGS. 50A and 50B depict three different representations of an intelligent alert configuration system overlayed upon several different aspects of an application in accordance with an embodiment of the present principles.

FIG. 51 depicts a view configuration page in accordance with an embodiment of the present principles.

FIG. 52 depicts a view configuration alerts and auto-tasks in accordance with another embodiment of the present principles.

FIG. 53 depicts a Data Command Center architecture and connectivity to external Health Information Technology systems and third-party services in accordance with an embodiment of the present principles.

FIG. 54 depicts examples of Health Information Technology systems in accordance with an embodiment of the present principles.

FIG. 55 depicts a medical records dashboard of a Data Command Center of the present principles that enables a healthcare provider while delivering medical care to a patient to participate in revenue cycle management in accordance with an embodiment of the present principles.

FIG. 56 depicts an embodiment of an in-context image management system of a Data Command Center of the present principles displaying thumbnailed images in rows and columns in accordance with an embodiment of the present principles.

FIG. 57 depicts an embodiment of the in-context image management system displaying images in the context of graphically visualized medical data modules in accordance with an embodiment of the present principles.

FIGS. 58A and 58B depict an embodiment of the in-context image management system displaying multiple patients and multiple images in accordance with an embodiment of the present principles.

FIGS. 59A, 59B, 59C, and 59D depict an embodiment of a display of an in-context image management system enabling a direct edit of image information in accordance with an embodiment of the present principles.

FIG. 60 depicts a high level diagram of a Financial Flowsheet in accordance with an embodiment of the present principles.

FIG. 61 depicts a flow diagram of a method for in-context display of images and patient related information in accordance with an embodiment of the present principles.

FIG. 62 depicts a high-level infrastructure diagram of a Data Command Center in accordance with an embodiment of the present principles.

FIG. 63 illustrates a logical implementation of a multitenant example of DHRpro with a Co-Management Connection to allow for sharing of data.

FIG. 64 illustrates examples of different data being accessed.

FIG. 65 Illustrates a standard workflow when data is received from an external system through any of the messaging standards or API methods in accordance with the tool described herein.

FIG. 66A depicts a first portion of a workflow diagram of a process for intelligently expanding, collapsing, highlighting, graying, displaying, and/or hiding columns, rows and/or any other portion of the medical records dashboard in accordance with an embodiment of the present principles.

FIG. 66B depicts a second portion of the workflow diagram of a process of FIG. 48A for intelligently expanding, collapsing, highlighting, graying, displaying, and/or hiding columns, rows and/or any other portion of the medical records dashboard in accordance with an embodiment of the present principles.

FIG. 66C depicts a third portion of the workflow diagram of a process of FIG. 48A for intelligently expanding, collapsing, highlighting, graying, displaying, and/or hiding columns, rows and/or any other portion of the medical records dashboard in accordance with an embodiment of the present principles.

FIG. 66D depicts a flow diagram of a method for rules-based data display in a data command center comprising a medical records dashboard in accordance with an embodiment of the present principles.

FIG. 67 illustrates examples of different ways data fields may be dynamically updated in accordance with an embodiment of the present principles.

FIG. 68 depicts a series of data points associated with the determination of how and when to display data fields.

FIGS. 69A and 69B depict configuration options associated with dynamic data representation.

FIGS. 70A1-70A2 depict a patient specific dashboard in accordance with an embodiment of present principals.

FIGS. 70B1-70B2 depict additional data display within a patient specific dashboard in accordance with an embodiment of present principals.

FIGS. 70C1-70C2 depict an ordering module within a patient specific dashboard in accordance with an embodiment of present principals.

FIGS. 70D1-70D2 depict a future order and filtering within patient specific dashboard in accordance with an embodiment of present principals.

FIGS. 70E1-70E2 depict in-context alert configuration within a patient specific dashboard in accordance with an embodiment of present principals.

FIGS. 70F1-70F2 depict a correlative line graph in accordance with an embodiment of present principals.

FIGS. 70G1-70G2 depict an augmented correlative line graph in accordance with an embodiment of present principals.

FIG. 71 illustrates dynamic, interactive header and summary rows in accordance with an embodiment of the present principles.

FIGS. 72A and 72B further illustrate dynamic, interactive header and summary rows in accordance with an embodiment of the present principles.

FIG. 73 illustrates an Ordering Panel with required Ordering Elements.

FIG. 74 illustrates generally the process for ordering a procedure in accordance with the tool described herein.

FIG. 75 illustrates generally the process for receiving an order in accordance with the tool described herein.

FIG. 76 illustrates the logic used to check for pre-authorization and/or a referral in accordance with the tool described herein.

FIG. 77 Illustrates the precertification process that is invoked by presentation of a CPT Procedure code and an ICD10 Diagnosis code in accordance with the tool described herein.

FIG. 78 Illustrates the referral process that is invoked by presentation of a CPT Procedure code and an ICD10 Diagnosis code in accordance with the tool described herein.

FIG. 79 illustrates an exemplary clinical decision support system in accordance with the tool described herein.

FIGS. 80A1 and 80A2 illustrate a dynamic report in accordance with present principals.

FIGS. 80B1 and 80B2 illustrate order initiation from a dynamic report in accordance with present principals.

FIGS. 80C1 and 80C2 illustrate order completion and display from a dynamic report in accordance with present principals.

FIGS. 81A1 and 81A2 depict an embodiment of a medical records dashboard of a Data Command Center in which reports can be seen in context with an ability to order in accordance with an embodiment of the present principles.

FIGS. 81B1 and 81B2 depict an embodiment of a medical records dashboard of a Data Command Center in which reports can be seen in context with an ability to order in accordance with an embodiment of the present principles.

FIGS. 81C1 and 81C2 depict an embodiment of a medical records dashboard of a Data Command Center in which reports can be seen in context with an ability to order in accordance with an embodiment of the present principles.

FIGS. 81D1 and 81D2 depict an embodiment of a medical records dashboard of a Data Command Center in which reports can be seen in context with an ability to order and view orders in accordance with an embodiment of the present principles.

FIG. 82A depicts a trigger processing module in accordance with principals denoted in FIG. 69A and FIG. 69B.

FIG. 82B illustrates an example of a critical patient indicator.

FIG. 83A illustrates a horizontal correlative graph in accordance with an embodiment of the present principles.

FIG. 83B illustrates a series of configurations in accordance with an embodiment FIG. 83t of the present principles.

FIG. 83C illustrates interactions between data sets in accordance with an embodiment of the present principles.

FIGS. 83D1 and 83D2 illustrate direct access and parameter setting on a horizontal graph in accordance with an embodiment of the present principles.

FIGS. 83E1 and 83E2 illustrate several views of limited area displays in accordance with an embodiment of the present principles.

FIG. 83F illustrates another view of limited area display in accordance with an embodiment of the present principles.

FIG. 84A illustrates a weighting system in accordance with an embodiment of the present principles.

FIG. 84B illustrates a weighting system flow diagram in accordance with an embodiment of the present principles.

FIG. 84C illustrates a whole life view zoomed out in accordance with an embodiment of the present principles.

FIG. 84D illustrates a whole life view zoomed in to a first level in accordance with an embodiment of the present principles.

FIG. 84E illustrates a whole life view fully zoomed in in accordance with an embodiment of the present principles.

FIG. 85A illustrates a high-level data knowledge storage system in accordance with an embodiment of the present principles.

FIG. 85B illustrates a high-level data categorization system in accordance with an embodiment of the present principles.

FIG. 85C illustrates a high-level data category search system in accordance with an embodiment of the present principles.

FIG. 85D illustrates a high-level data correlation system in accordance with an embodiment of the present principles.

FIG. 86 illustrates a first Flowsheet that can be used in predictive analytics in accordance with an embodiment of the present principles.

FIGS. 87A, 887B, 87C, 87D, and 87E illustrate a second Flowsheet that can be used in predictive analytics in accordance with an embodiment of the present principles.

FIGS. 88A and 88B illustrate predictive analytics in accordance with an embodiment of the present principles.

FIGS. 89A, 89B, and 89C illustrate predictive analytics in accordance with an embodiment of the present principles.

FIG. 90 illustrates predictive analytics in accordance with an embodiment of the present principles.

FIG. 91 illustrates predictive analytics in accordance with an embodiment of the present principles.

FIG. 92 illustrates predictive analytics in accordance with an embodiment of the present principles.

The figures are not drawn to scale and may be simplified for clarity. It is contemplated that elements and features of one embodiment may be beneficially incorporated in other embodiments without further recitation.

### DETAILED DESCRIPTION

Embodiments of the present principles generally relate to a Data Command Center for displaying data on a display screen from multiple data sources and enabling navigation amongst the data on a single display. While the concepts of the present principles are susceptible to various modifications and alternative forms, specific embodiments thereof are shown by way of example in the drawings and are described in detail below. It should be understood that there is no intent to limit the concepts of the present principles to the particular forms disclosed. On the contrary, the intent is to cover all modifications, equivalents, and alternatives consistent with the present principles and the appended claims. For example, although embodiments of the present principles will be described primarily with respect to inter-function with an EMR system, such teachings should not be considered limiting. Embodiments in accordance with the present principles can inter-function with other informational systems such as Health Information Exchanges (HIEs), Billing Clearinghouses, Insurance Companies, Picture Archiving and Communication Systems (PACS) as well as third party services and the like.

In addition, the tool embodiments of the present principles are not limited in application to the details of construction and the arrangement of components set forth in the following description or illustrated in the following drawings. Embodiments of the present principles are capable of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, the term “medical care provider” is intended to represent any healthcare provider/clinical professional such as a doctor, physician, podiatrist, chiropractor, dentist, veterinarian, ancillary staff, nurses, physician's assistant, medical care provider, physical therapist, all allied health professionals, and/or hospital staff member. All such healthcare providers/clinical professional can implement embodiments of the present principles the tool as interchangeable users.

As used herein a row, column, or line of items (even a diagonal line) is intended to represent a sequencing or evaluation of information in any direction. In the embodiments depicted herein, information does not have to be depicted as having a visual or physical separation in the vertical or horizontal direction to be defined as being a row or column. In accordance with the present principles items next to each other horizontally, lined up in such a way that straight lines above and below can be drawn and items fall between those two horizontal lines, can be considered as being in a row. Items in rows can be related by similar time or other common or same denominator, such as a medical service, procedure, image or financial number, so that a user can quickly visualize trends or changes in those items. Similarly, items next to each other vertically, lined up in such a way that straight lines to the left and to the right can be drawn can be considered as being in a column. In some embodiments, items can be arranged diagonally and be considered to be in a row or a column.

As used herein, Practice Management Systems (PMs) are programs that perform the billing collection and reconciliation of payments as well as scheduling patients. PMs can also be referred to as Revenue Cycle Management (RCM) and have associated billing companies that use software to help practices and medical care providers get the bills out and collect money from insurance companies. In some embodiment, these entities can integrate with and work through clearing houses.

In the embodiments described herein, the terms window screen, scrolling screen, display. view, snapshot and the like can be used interchangeably and are intended to represent a single instance of the presentation of medical information associated with at least one patient. In the described embodiments, the single instance can be presented on one or more windows, in a single or multiple screens, a scrolling screen, in one or more views and using one or more snapshots. For example, in some embodiments in accordance with the present principles a user can access different panels from a scrolling screen and converge the panels into a single view or snapshot. That is, in accordance with the present principles, a user is able to compile data/information from various windows, screens, scrolling screens, displays, snapshots and the like and create a single instance presentation including the data/information of interest to the user for at least one patient. In accordance with the present principles, a single instance presentation can be presented on more than one monitor at a time. As used herein, the term single instance presentation is intended to describe a single display interface that is not limited to a single monitor. That is, in some embodiments, what defines a single instance presentation is the fact that there is a single interface, a single control that controls the presentation of the date/information, which can be then be viewed on one or more monitors or other means.

The term medical tests as described herein is intended to describe medical procedures performed for or on patients including but not limited to image or imaging, diagnostic tests, radiological tests or procedures, laboratories, chemistry and hematological tests, photography, genetic testing, nuclear scans, ultrasounds, x-rays, optical coherent tomography photographs and angiographies, assessments and plans, letters, examination findings and any medical testing or medical services that tests or screens patients for a medical condition, which in some instance can be identified by CPTcodes. It should be further noted that in some instances, terms like diagnosis can be reflected by ICD 9 or 10 or similar identifying factors, and medications can be interchangeable.

As used herein, the terms icon, symbol, and indicator are all interchangeable and are intended to describe a visual element enabling the access of additional underlying information and having the ability to convey additional information simply by their presentation. That is, such visual elements can convey information by their display which can include such visual presentations including but not limited to words, numbers, blinking elements, flashing elements, color changing elements, elements in italics, underlined elements, and the like or any means that draws the attention of a user.

The reference to a medical records dashboard of the present principles described throughout the teachings herein is intended to refer to any embodiment of a medical records dashboard according to the present principles that is applicable to a currently described embodiment.

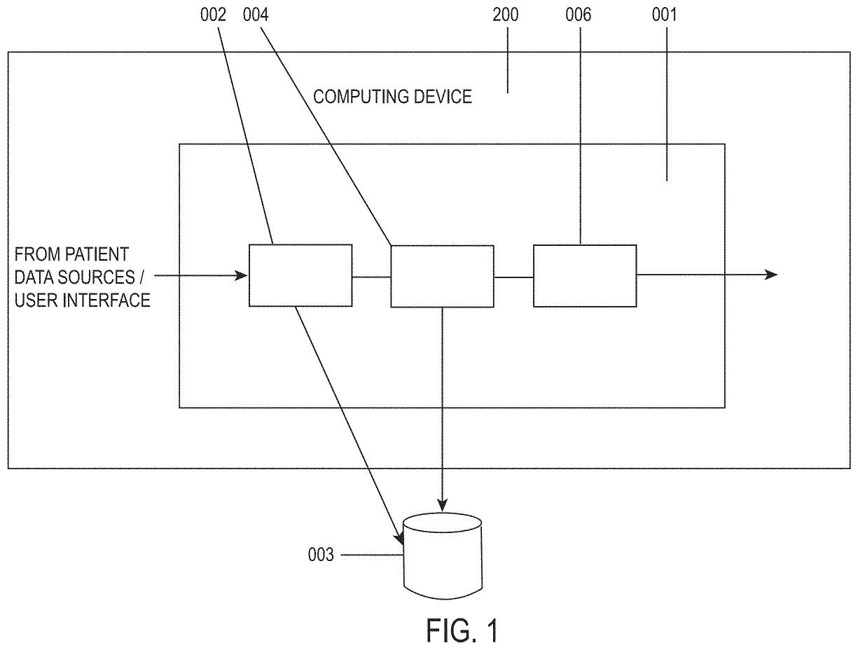

FIG. 1 depicts a high-level block diagram of a Data Command Center (DCC) 001 in accordance with an embodiment of the present principles. In the embodiment of FIG. 1, the Data Command Center 001 illustratively comprises an integration module 002 (i.e., to interface data between an EMR and the DCC), a Rules module 004 (i.e., to determine where and how the data is to be displayed), and a display module 006 (i.e., to display the data in the appropriate place). In the embodiment of FIG. 1, the integration module 002 and the rules module 004 can be in communication with a data storage 003. For example, the integration module 002 can store data from patient data sources in the data storage 003 and the rules module 004 can access the data storage 003 to retrieve data and/or information stored therein.

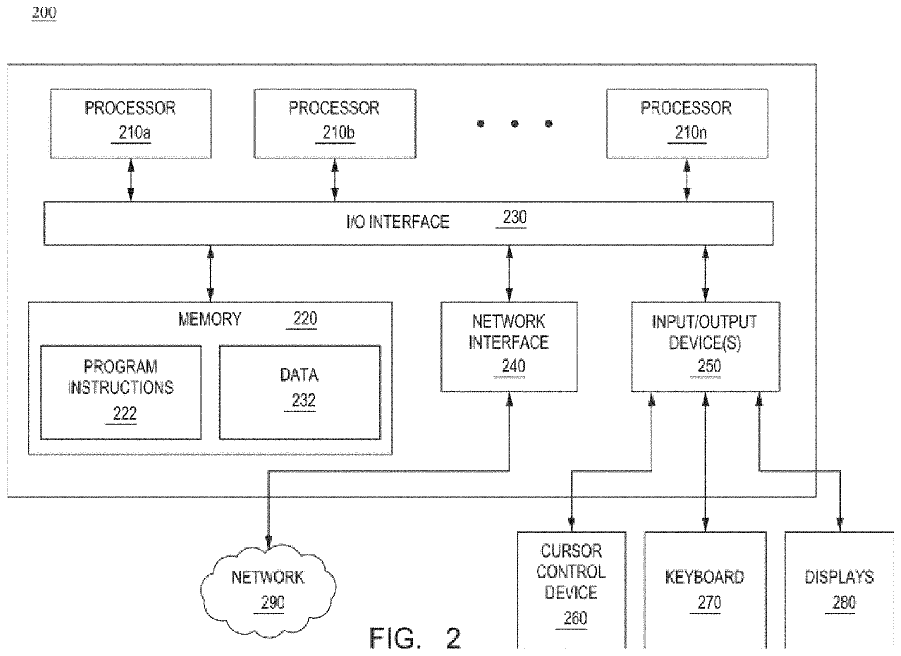

As depicted in FIG. 1, embodiments of a Data Command Center in accordance with the present principles, such as the Data Command Center 001 of FIG. 1, can be implemented in a computing device 200. FIG. 2 depicts a high-level block diagram of a computing device 200 suitable for use with embodiments of a Data Command Center in accordance with the present principles such as the user Data Command center 001 of FIG. 1. In some embodiments, the computing device 200 can be configured to implement methods of the present as processor-executable executable program instructions 222 (e.g., program instructions executable by processor(s) 210) in various embodiments.

In the embodiment of FIG. 2, the computing device 200 includes one or more processors 210a-210n coupled to a system memory 220 via an input/output (I/O) interface 230. The computing device 200 further includes a network interface 240 coupled to I/O interface 230, and one or more input/output devices 250, such as cursor control device 260, keyboard 270, and display(s) 280. In various embodiments, a user interface can be generated and displayed on display 280. In some cases, it is contemplated that embodiments can be implemented using a single instance of computing device 200, while in other embodiments multiple such systems, or multiple nodes making up the computing device 200, can be configured to host different portions or instances of various embodiments. For example, in one embodiment some elements can be implemented via one or more nodes of the computing device 200 that are distinct from those nodes implementing other elements. In another example, multiple nodes may implement the computing device 200 in a distributed manner.

In different embodiments, the computing device 200 can be any of various types of devices, including, but not limited to, a personal computer system, desktop computer, laptop, notebook, tablet or netbook computer, mainframe computer system, handheld computer, workstation, network computer, a camera, a set top box, a mobile device, a consumer device, video game console, handheld video game device, application server, storage device, a peripheral device such as a switch, modem, router, or in general any type of computing or electronic device.

In various embodiments, the computing device 200 can be a uniprocessor system including one processor 210, or a multiprocessor system including several processors 210 (e.g., two, four, eight, or another suitable number). Processors 210 can be any suitable processor capable of executing instructions. For example, in various embodiments processors 210 may be general-purpose or embedded processors implementing any of a variety of instruction set architectures (ISAs). In multiprocessor systems, each of processors 210 may commonly, but not necessarily, implement the same ISA.

System memory 220 can be configured to store program instructions 222 and/or data 232 accessible by processor 210. In various embodiments, system memory 220 can be implemented using any suitable memory technology, such as static random-access memory (SRAM), synchronous dynamic RAM (SDRAM), nonvolatile/Flash-type memory, or any other type of memory. In the illustrated embodiment, program instructions and data implementing any of the elements of the embodiments described above can be stored within system memory 220. In other embodiments, program instructions and/or data can be received, sent or stored upon different types of computer-accessible media or on similar media separate from system memory 220 or computing device 200.

In one embodiment, I/O interface 230 can be configured to coordinate I/O traffic between processor 210, system memory 220, and any peripheral devices in the device, including network interface 240 or other peripheral interfaces, such as input/output devices 250. In some embodiments, I/O interface 230 can perform any necessary protocol, timing or other data transformations to convert data signals from one component (e.g., system memory 220) into a format suitable for use by another component (e.g., processor 210). In some embodiments, I/O interface 230 can include support for devices attached through various types of peripheral buses, such as a variant of the Peripheral Component Interconnect (PCI) bus standard or the Universal Serial Bus (USB) standard, for example. In some embodiments, the function of I/O interface 230 can be split into two or more separate components, such as a north bridge and a south bridge, for example. Also, in some embodiments some or all of the functionality of I/O interface 230, such as an interface to system memory 220, can be incorporated directly into processor 210.

Network interface 240 can be configured to allow data to be exchanged between the computing device 200 and other devices attached to a network (e.g., network 290), such as one or more external systems or between nodes of the computing device 200. In various embodiments, network 290 can include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 240 can support communication via wired or wireless general data networks, such as any suitable type of Ethernet network, for example; via digital fiber communications networks; via storage area networks such as Fiber Channel SANs, or via any other suitable type of network and/or protocol.

Input/output devices 250 can, in some embodiments, include one or more display terminals, keyboards, keypads, touchpads, scanning devices, voice or optical recognition devices, or any other devices suitable for entering or accessing data by one or more computer systems. Multiple input/output devices 250 can be present in computer system or can be distributed on various nodes of the computing device 200. In some embodiments, similar input/output devices can be separate from the computing device 200 and can interact with one or more nodes of the computing device 200 through a wired or wireless connection, such as over network interface 240.

Those skilled in the art will appreciate that the computing device 200 is merely illustrative and is not intended to limit the scope of embodiments. In particular, the computer system and devices can include any combination of hardware or software that can perform the indicated functions of various embodiments, including computers, network devices, Internet appliances, PDAs, wireless phones, pagers, and the like. The computing device 200 can also be connected to other devices that are not illustrated, or instead can operate as a stand-alone system. In addition, the functionality provided by the illustrated components can in some embodiments be combined in fewer components or distributed in additional components. Similarly, in some embodiments, the functionality of some of the illustrated components may not be provided and/or other additional functionality can be available.

The computing device 200 can communicate with other computing devices based on various computer communication protocols such a Wi-Fi, Bluetooth® (and/or other standards for exchanging data over short distances includes protocols using short-wavelength radio transmissions), USB, Ethernet, cellular, an ultrasonic local area communication protocol, etc. The computing device 200 can further include a web browser.

Although the computing device 200 is depicted as a general purpose computer, the computing device 200 is programmed to perform various specialized control functions and is configured to act as a specialized, specific computer in accordance with the present principles, and embodiments can be implemented in hardware, for example, as an application specified integrated circuit (ASIC). As such, the process steps described herein are intended to be broadly interpreted as being equivalently performed by software, hardware, or a combination thereof.

Those skilled in the art will also appreciate that, while various items are illustrated as being stored in memory or on storage while being used, these items or portions of them can be transferred between memory and other storage devices for purposes of memory management and data integrity. Alternatively, in other embodiments some or all of the software components can execute in memory on another device and communicate with the illustrated computer system via inter-computer communication. Some or all of the system components or data structures can also be stored (e.g., as instructions or structured data) on a computer-accessible medium or a portable article to be read by an appropriate drive, various examples of which are described above. In some embodiments, instructions stored on a computer-accessible medium separate from the computing device 200 can be transmitted to the computing device 200 via transmission media or signals such as electrical, electromagnetic, or digital signals, conveyed via a communication medium such as a network and/or a wireless link. Various embodiments can further include receiving, sending or storing instructions and/or data implemented in accordance with the foregoing description upon a computer-accessible medium or via a communication medium. In general, a computer-accessible medium can include a storage medium or memory medium such as magnetic or optical media, e.g., disk or DVD/CD-ROM, volatile or non-volatile media such as RAM (e.g., SDRAM, DDR, RDRAM, SRAM, and the like), ROM, and the like.

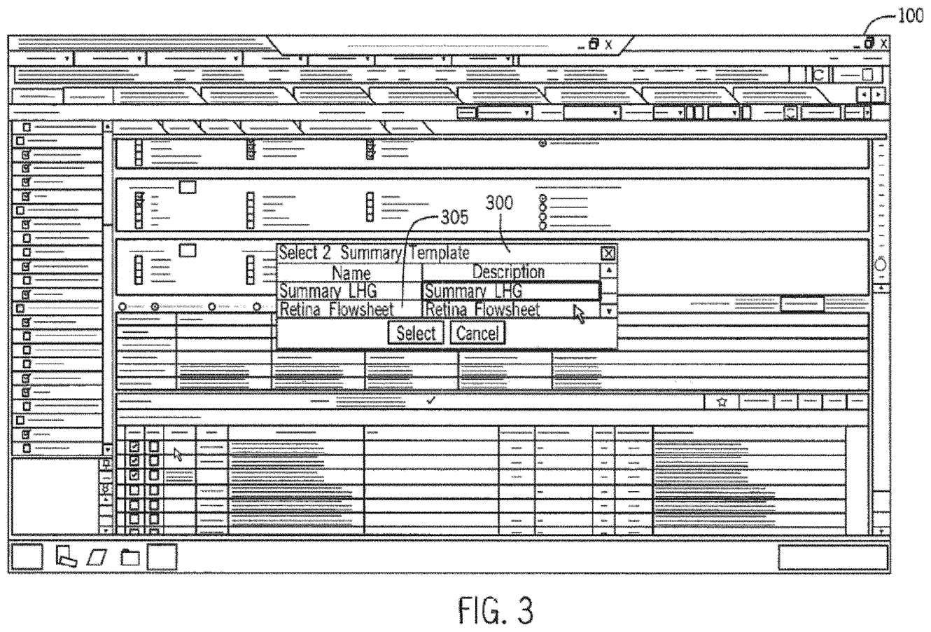

In some embodiments, a Data Command Center (CC) in accordance with the present principles is implemented as a data interface to a medical record system (e.g., EMR). In such embodiments a medical care provider can utilize a conventional medical record system to launch or enter a Data Command Center (CC) in accordance with the present principles including a medical-services tracking system that can display information dashboards, tables, charts, windows, as will be described herein. For example, FIG. 3 depicts a high-level diagram of a medical records dashboard selection window of, for example, a medical record system useful for selecting and launching at least a portion of a Data Command Center (CC) in accordance with an embodiment of the present principles. For example, as depicted in FIG. 3, a medical records dashboard selection window 300 can include one or more selectable medical records dashboards from which a user can select to access at least one medical records dashboard. For example, in one non-limiting example embodiment, the user can select a “Retina Flowsheet” 305 to access and/or launch a medical records dashboard including a retina flowsheet in a Data Command Center (CC) in accordance with the present principles. In some further embodiments, the at least one medical records dashboard can include any number of selectable medical records for any medical condition, and/or any medical diagnosis, and/or any medical treatment.

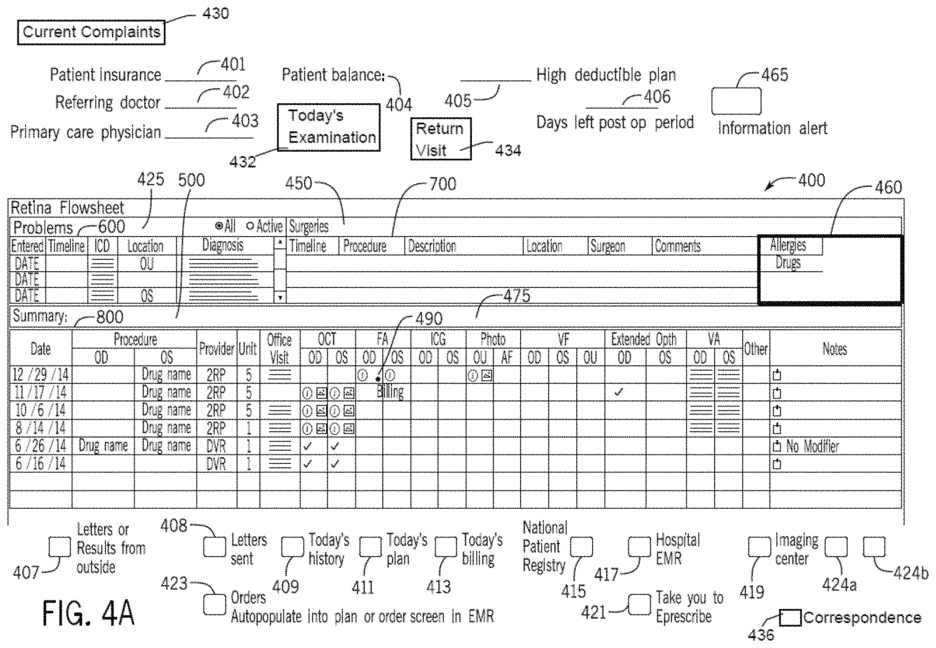

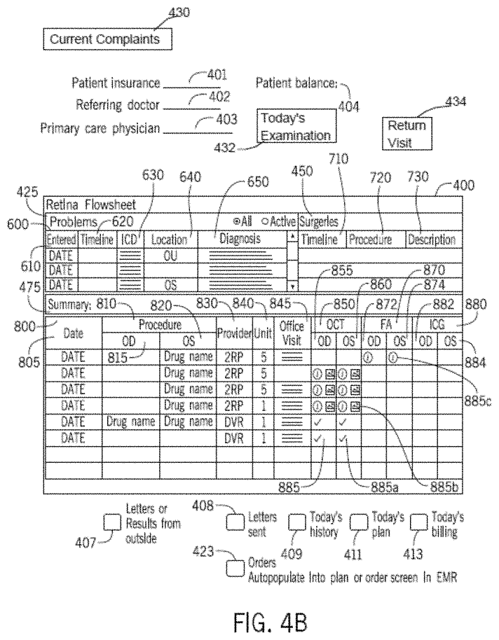

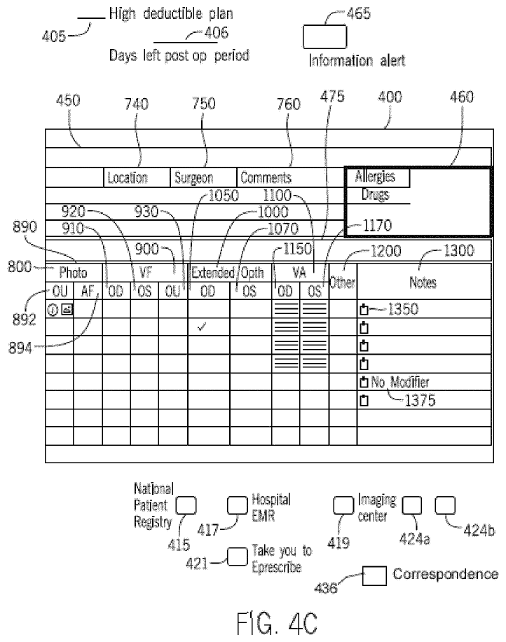

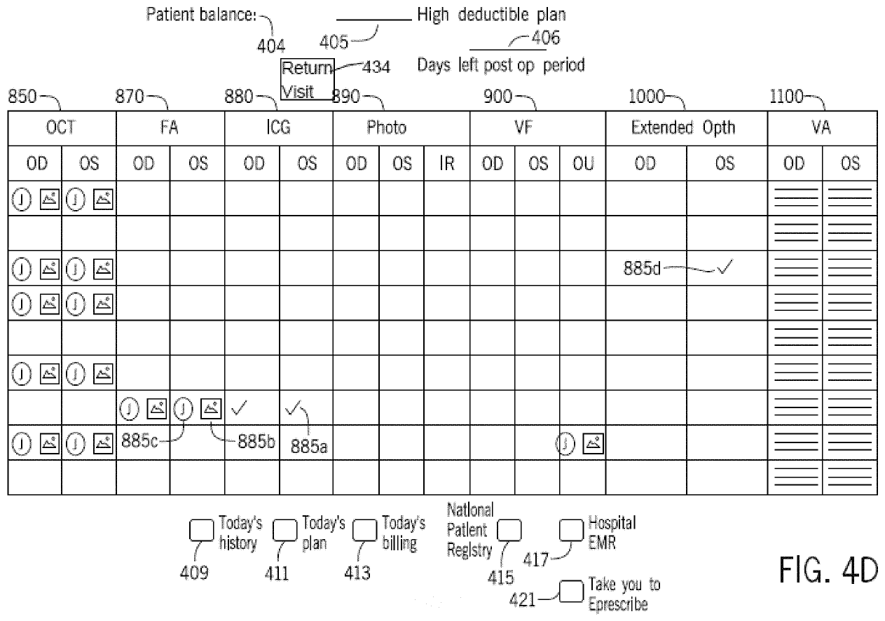

For example, FIG. 4A depicts a medical records dashboard 400 of the Data Command center 001 in accordance with an embodiment of the present principles. The medical records dashboard 400 is capable of displaying data from one or more medical records, and/or track medical procedures and services based on claims made or billing signed off by a physician for one or more delivered medical procedures or services. For example, in some embodiments the integration module 002 of the Data Command center 001 can dynamically link to various external databases comprising patient information that can be displayed in the medical records dashboard 400 by the display module 006 in accordance with rules for display in the rules module 004. For example, in some embodiments, the Data Command center 001 can function as a portal to patient information prepared by the user or patient information from other sources.

In some embodiments, the medical records dashboard 400 can be auto-populated by the display module 006 in accordance with rules in the rules module 004 as a function of claims made or billing signed off by a physician. In such embodiments, any data displayed within the medical records dashboard 400 is derived from one or more claim records that have been billed for one or more procedures or services have previously been provided to the patient. In some other embodiments, auto-population can be enabled in both directions interacting as a switchboard between the entire EMR and the medical records dashboard 400 along with what is added to any window, sub-window, column or entry in the medical records dashboard 400 being automatically added to the appropriate part of the chart for documentation before finalizing the encounter.

The medical records dashboard 400 can display information related to any medical procedures or services in relation to care of a patient. For example, in some embodiments, the medical records dashboard 400 can display information related to medical procedures or services in relation to retinal eye medical care of a patient. In some embodiments, the medical records dashboard 400 can display information including components where there is a summary of the patient's problem list that a user can input patient information and constantly update and change. Further, this information can be auto-populated with the touch of a button into a designated location such as the current plan documenting the patient's current visit (thus aiding documentation for the current visit). Further, whatever is important for a user to input into the day's visits for documentation can be initially inputted in the table, and then permanently into the day's patient visits. Further, a summary section of the medical records dashboard 400 can be dynamic and can be changed at every visit rather than being written to an unchangeable document or file (e.g., such as a PDF). Further, any patient data that is input, received, analyzed, or created can be auto-populated into any portion of the dashboard 400, and/or can form a dataflow out of the medical records dashboard 400 to another electronic system or server, or another user, observer, or other third-party.

In some embodiments, the medical records dashboard 400 can display various windows and sub-windows based on a user preference and/or current or previous user interaction with the medical records dashboard 400. For example and with reference to FIG. 4A, in some embodiments, the medical records dashboard 400 can display a problems window 425 and/or a surgeries window 450 where information related to a patient's medical problems and surgeries can be displayed in information columns 600, 700 respectively. Further, in some embodiments, patient information related to allergies and drugs can be displayed within the allergies/drug section 460. This information can be auto-populated from a variety of sources, or inputted by a user.

In some embodiments, the medical records dashboard 400 can include a summary window 475 enabling a user to view and edit summary information related to the patient, any details of care provided to the patient, and/or and any medical diagnosis information prepared by a medical practitioner. Further, in some embodiments, the medical records dashboard 400 can also display detailed information related to any medical procedures or services provided to the patient, including procedures or services that are auto-populated by claims made, or billings or payments including billing signed off by a physician as detailed above. For example, in some embodiments, the medical records dashboard 400 can display visual display window 500 including information columns 800 that can be auto-populated by claims made or billings signed off by a physician. The auto-population can include billings, payments, or other information from anywhere in the EMR chart. For example in some embodiments, the information that is auto-populated can include treatment summaries, and/or diagnosis summaries, and/or patient feedback summaries, and/or other physician summaries, and so on. For example, in some embodiments, the Data Command center 001 can display and/or auto-populate at least one field, table, or window with at least one of a patient's prior medical procedures, diagnostic tests, surgeries, current medications, current illnesses, treated illnesses, and so on. The Data Command center 001 can auto-populate various data fields via an electronic dataflow established between the Data Command center 001 and one or more computer systems of servers that comprise patient information (e.g., such as electronic medical records). The dataflow can comprise a two-way flow from the source of patient data to the Data Command center 001 and from the Data Command center 001 to the source. In some embodiments, this information can be any medical diagnosis information, any medical procedures or services provided to the patient, procedures or services by claims made, or billings or payments including billing signed off by a physician as detailed earlier, any information from anywhere in the EMR chart including treatment summaries, and/or diagnosis summaries, the patient's prior medical procedures, diagnostic tests, surgeries, current medications, current illnesses, treated illnesses, and/or patient feedback summaries, and/or other physician summaries, patient outcome summaries, treatment summaries, and/or diagnosis summaries, and/or patient feedback summaries, and/or other physician summaries or treatments. Further, in some embodiments, the information that is auto-populated can include patient outcome summaries. For example, in some embodiments, the Data Command center 001 processes a plurality of patient outcomes and displays an analysis of patient outcomes based at least in part on patient information from treatment summaries, and/or diagnosis summaries, and/or patient feedback summaries, and/or other physician summaries or treatments. In some embodiments, the patient outcomes can include or comprise physician quality reporting system (PQRS) quality measures. In some embodiments, calculated or reported patient outcomes can include or comprise at least one PQRS measures code.

As depicted in FIG. 4A, the medical records dashboard 400 can include miscellaneous information identifying the patient, information related to the patient's insurance plan, physicians and referring physicians, and the patient's current balance. Other information can relate to the patient's prior visit, prior diagnosis or procedure and any important information relevant to the next visit. Additional information can relate to the current visit, including history of illness and chief or current medical complaint, billing information, and retrievable medical information including pharmacy information. For example and as depicted in FIG. 4A, in some embodiments, the medical records dashboard 400 can include a patient insurance entry 401, referring physician entry 402, and primary care physician entry 403. The medical records dashboard 400 can also include patient balance entry 404, and a high deductible plan entry 405. Important patient information related to a pending or current visit can include a “days left post-op period” entry 406 and/or an information alert 465. In some embodiments, the information alert 465 can be auto-populated based on other information or entries in the medical records dashboard 400. In other embodiments, the information alert 465 can be set by any user to alert the user or other user of information relevant to the patient. In some embodiments, the information alert 465 can comprise a daily technician update, including information to medical information such as blood pressure, or whether the patient is pregnant, or any other urgent information with which a member of a health care team can alert another member. Further, this information can become permanent or can be deleted from the medical records dashboard 400, and from any record or table accessible from the medical records dashboard 400, including any medical record. Further, this information can serve as or be configured as a “sticky note” that can be removed from any of the above-mentioned records. For example, the “sticky note” can be an electronic sticky note riding on the dashboard or any record accessible from the dashboard.

Furthermore, the medical records dashboard 400 can provide improvement as described where test interpretations and evaluation of patients, once documented and billed, usually become date stamped, and cannot be easily amended without applying a new date of amendment. In some embodiments, the Data Command center 001 can improve and follow care that will not necessarily be used as part of a particular day's medical record. Therefore, months or years apart, physicians can add notes into the table when new findings, discoveries, or realizations warrant it without feeling encumbered that they are “changing past medical record” and a disclosure of such can be at the bottom of the medical records dashboard 400. Allowing physicians and technicians to add and change notes within the medical records dashboard 400 (rather than changing a patient's EMR chart) can enable a user to summarize critically important health/history/treatment data, which can then be used as a faster point of reference while examining the patient. Notes that exist on the medical records dashboard 400 can flag or alert a user to an important medical change, and can be used as an additional form of communication to strengthen lines of communication between technicians/clinic staff and physicians to better ensure that a medical care provider is quickly directed to important medical information.

In some embodiments, a daily technician update can be accessed or otherwise made visible to the user in at least one portion of the dashboard 400. In some embodiments, the information alert 465 can be displayed in a specific color and/or with a specific graphic and/or animation. For example, in some embodiments, the information alert 465 can comprise a flashing red animation. To protect the medical care provider during an audit, a statement on the medical records dashboard 400 can be added that “notes on this table” are not necessarily added at the time listed as the date and not for documentation in a medical record, but as a rapid reminder medical decision making and cliff note reference tool. As another example, if this patient's records were ever sent to another medical care provider or insurance company or were audited, this is critical information that a medical care provider is often not privy to and an icon on the table will alert the physician of this fact. By selecting this or another icon, the history of this audit or records release request or other occurrence can be seen. So, if an insurance company is requesting a medical necessity report or other information that is needed by a billing office or anyone else, the medical care provider can be informed on the medical records dashboard 400 so that the medical care provider can instantly decide what is needed.

As depicted in FIG. 4A, the medical records dashboard 400 can include an icon 407 enabling access to one or more letters or results from external data sources or third parties. In some embodiments, the medical records dashboard 400 can further include an icon 408 enabling access to letters sent 408, which can be written, typed, and/or dictated from the user and/or another medical care provider. In some embodiments, the medical records dashboard 400 includes an entry or access to the current day's history, the current day's plan, and/or to the current day's billing. For example, as depicted in FIG. 4A, the medical records dashboard 400 includes a “Today's history” button or icon 409, a “Today's plan” button or icon 411, and a “Todays billing” button or icon 413. In some embodiments, the medical records dashboard 400 can include a correspondence button or icon 436, which can be used to view, access, enter, and/or auto-populate correspondence related to a patient's care. Such correspondence can include any medical record and/or any correspondence generated while the patient is under care by the user and/or any other physician or medical practitioner, medical services provider, and/or medical insurance company.

In some embodiments, the medical records dashboard 400 can display a summary of the patient's problem list in which a user can input patient information and constantly update and change. For example, the medical records dashboard 400 of FIG. 4A includes an icon/button 430 for enabling the entry of or access to current complaints of the patient. In some embodiments, if a user activates (e.g., by clicking using a cursor) the button 430, information related to the patient's current medical problems or complaints can be shown and/or displayed and/or updated by a user. In some embodiments, the information can be auto-populated into the medical records dashboard 400.

In some embodiments, the medical records dashboard 400 can include a today's examination access icon/button 432 enabling a user to access, view and/or input patient information, patient examination results, tests, notes, or any information relevant to the medical care of the patient. By activating (e.g., by clicking using a cursor) today's examination access icon/button 432, information related to the patient's examination including medical problems or complaints patient information, patient examination results, tests, notes, etc., can be presented and/or displayed and/or updated using one or more windows and the like. In some embodiments, the information associated with the today's examination access icon/button 432 can be auto-populated into the medical records dashboard 400 by, for example, the display module 006 following auto-population rules of the rules module 004.

In some embodiments, any stored or displayed patient's examination records/data can be cleared from the medical records dashboard 400 following some time period once a patient visit is complete. In some embodiments, the medical records dashboard 400 can remove the display of or access to a previous patient's examination details once the patient visit has ended. In some embodiments, the medical records dashboard 400 can remove display or access to a previous patient's examination details later in the day of the patient's visit, or before the following day, or at any time selected by the user. In some embodiments, the information can be auto-populated into any EMR system for recordation into one or more EMR's of the patient. In some embodiments, for any auto-populated information that includes technical information without any associated professional interpretation, the Data Command center 001 via the medical records dashboard 400 can provide a visual and/or audible alert to enable a user to provide an update for auto-population to an EMR system.

In some embodiments, the medical records dashboard 400 can include at least one link to information from external databases, providers, hospitals (e.g., such as a discharge summary), clinics and/or testing laboratories, etc., (e.g., where the information can include the overall diagnostic imaging center of the practice for certain pieces of equipment and into the machine to actually see all of the study). In the latter example, the medical records dashboard 400 can receive information from at least one coupled database and/or server and/or controller. For example, as depicted in the embodiment of FIG. 4A, the Data Command center 001 via the medical records dashboard 400 can have entry or access to third party data sources via icons/buttons, including but not limited to, the National Patient Registry icon/button 415, the hospital EMR icon/button 417, the imaging center icon/button 419 and the ePrescribe icon/button 421.

In some embodiments, orders can be auto-populated into the medical records dashboard 400 or order screen of an EMR using, for example, the Orders icon/button 423. For example, in some embodiments, during or after completion of a patient examination, any medical service, medical test or diagnostic, or other medical service can be auto-populated into an order section of the medical records dashboard 400. Any recommendation for a return visit can be viewed, accessed, and/or auto-populated using the return visit icon/button 434 of the medical records dashboard 400. For example, in some embodiments, the recommendations can be any advised next steps in the patient's care, any diagnosis, prescriptions, tests, etc. In some embodiments, the aforementioned “Today's plan” icon/button 411 can be used to view, access, and/or auto-populate details including for a day's activities for the patient examination.

In some embodiments, an “Imaging Center” icon/button 424a of the medical records dashboard 400 enables a user access to the piece or pieces of diagnostic equipment that were used that or another day for performing tests on a patient(s) so the user can now measure and/or access the test results. Such functionality can be internal to the user's practice so that any diagnostic equipment can be accessed. The ability to access the diagnostic equipment and data directly, in accordance with the present principles, enables a user access to not just one single piece of diagnostic equipment but all equipment available and all tests available can be evaluated and the evaluation of changes of such tests overtime can be made.

In the embodiment of the medical records dashboard 400 of FIG. 4A, the icon/button 424b enables a user access to a company sponsoring a clinical research website (i.e., sometimes a pharmaceutical company and other times a company that invented a device). Such functionality enables a clinical researcher access to such a website and input any data that was obtained from a patient visit using the access provided using the access provided. In a research application in accordance with the present principles, a researcher is provided access to diagnostic equipment and/or to a research spreadsheet where the researcher can access and input data.

Further details of the problems window 425, surgeries window 450, and command center visual display window 500 are provided in FIGS. 4B-4D illustrating enlarged views of portions of the medical records dashboard 400. For example, FIG. 4B depicts a portion of the medical records dashboard 400 of FIG. 4A in accordance with some embodiments of the present principles. As illustrated in FIG. 4B, in some embodiments, the information columns 600 of the problems window 425 can include a date column 610, a timeline column 620, an “ICD” column 630 for international classification of disease codes including international classification of disease codes, such as version 9 or version 10, (hereinafter collectively referred to as “ICD code” information), location of the problem or disorder (shown as “OD”, “OS”, “OU” identifying right eye, left eye, both eyes), or from any part of the body, and a diagnosis column 650 for detailing information related to an initial diagnosis or final diagnosis of a patient's problem or disorder that can be auto-populated or input manually. Further, in some embodiments, the information columns 700 of the surgeries window 450 can include information related to services or procedures that were provided to the patient (procedure columns 720), a description of the services or procedures performed (description columns 730), and when the services or procedures were provided (timeline columns 710).

FIG. 4C depicts greater detail of at least some portions of the medical records dashboard 400 of FIG. 4A in accordance with some embodiments of the present principles. Referring to FIG. 4C, in some embodiments, the surgeries window 450 can include location information 740, surgeon or physician information 750, and a comments section 760. Referring to the display window 500 of FIG. 4C, the information columns 800 can include a date column 805, and a procedure column 810 illustrating or providing access to information detailing one or more procedures performed on the patient. Further, the procedure column 810 can include an “OD” column 815, and “OS” column 820 providing right and left eye procedure information, or could be a body part (i.e., orthopedic surgery limb versus spine). In some embodiments, information related to the medical care provider, the location where the procedure was performed, and office visit information can be provided to the user in column 830, and unit column 840, and office visit column 845.

FIG. 4D depicts greater detail of at least some portions of the medical records dashboard 400 of FIG. 4A in accordance with some embodiments of the present principles. Referring to FIG. 4D, in some embodiments the user can view information related to tests and procedures performed on the patient. For example, in some embodiments, such information can include information related to one or more medical imaging procedures such as an optical coherence tomography (“OCT”), or fluorescein angiography (“FA”), and/or indocyanine green chorioangiography (“ICG”), or any current procedural terminology code (hereinafter “CPT code”), including any CPT code found in the American Medical Association CPT 2015 professional edition or other edition, the entire contents of which is incorporated by reference. Moreover, the user can view information related to tests and procedures performed on the patient based on an ICD code. Other clinical vocabularies such as Systematized Nomenclature of Medicine (hereinafter “SNOMED codes”) can be used in other embodiments as the system is not limited.

In some embodiments, the user can compare patient clinical information, such as labs and vitals using the medical records dashboard 400, before and after a selected medication has been prescribed or a procedure has been performed to better understand the effect of the medication or procedure on the patient. Similarly, in other embodiments, the user can examine how the current patient compares against other patients in the practice and population in general using the medical records dashboard 400 to better understand outcomes.

As depicted in FIGS. 4A-4D, in some embodiments, medical procedures performed (including any of the aforementioned medical imaging procedures) that have been billed and claimed can be viewed or accessed by a user within any of the “OCT” column 850 (split as an “OD” column 855 and “OS” column 860), an “FA” column 870 (split as an “OD” column 872 and “OS” column 874), and/or “ICG” column 880 (split as “OD” column 882 and “OS” column 884).

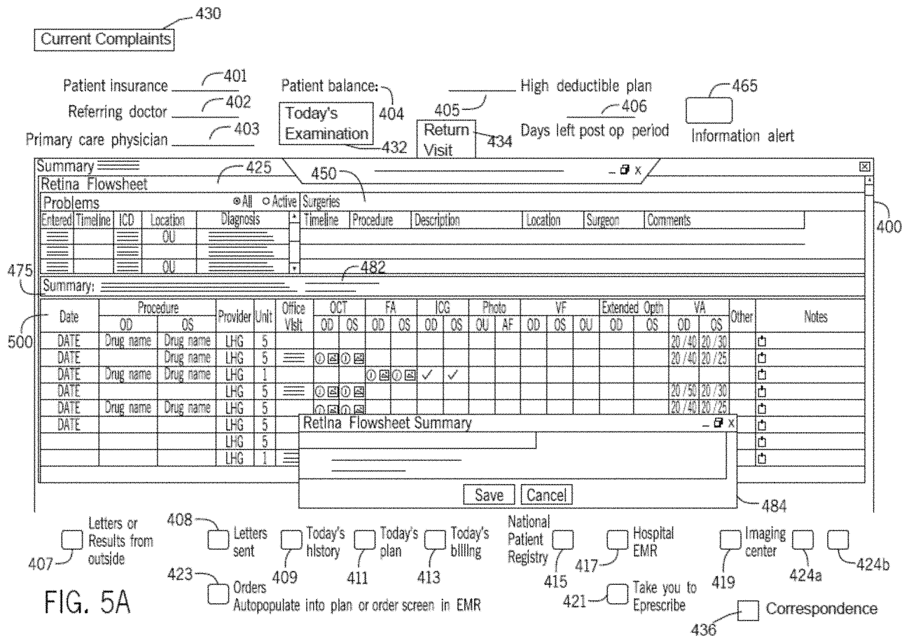

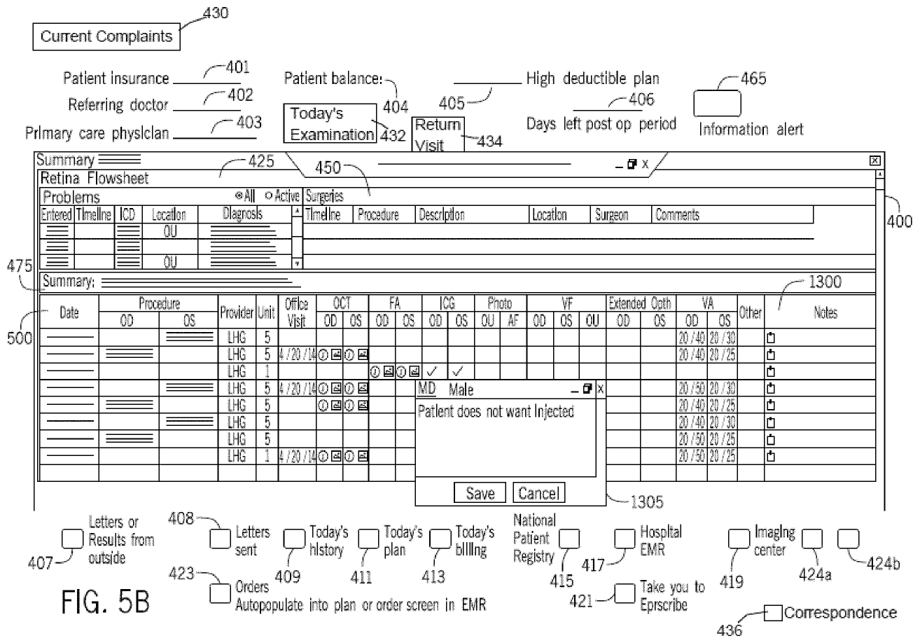

Referring back to FIG. 4C, the information columns 800 can include a photo column 890 configured to enable a user to access any photographic images of the patients eyes including optical and auto-fluorescent images of the eyes (“OU” column 892 and “AF” column 894). In some embodiments, if visual function tests were performed, information can be viewed or accessed in the visual field “VF” column 900 (including an “OD” column 910, “OS” column 920, and/or “OU” column 930). Some embodiments also include an extended ophthalmology column 1000 (including “OD” column 1050 and “OS” column 1070), and a visual acuity column (“VA” column 1100, including “OD” column 1150, and “OS” column 1170). In some embodiments, as described earlier, other details of various tests, procedures or services can be viewed or accessed in the other column 1200. Further, information associated with any of the user-accessible tests, procedures or services or other notes provided by the user and/or medical care provider can be viewed or accessed in the notes column 1300 using one or more notes access icons/buttons 1350 and/or by viewing a note entry 1375 (e.g., and/or any note entered using the note entry window 1375 . . . . The functionality of the notes column 1300 is further discussed below with reference to FIG. 5B). In accordance with embodiments of the present principles, the information can be auto-populated into the medical records dashboard 400 or into EMR plan pages as described above with respect to different embodiments. That is, as described above, various data fields/entries of the medical records dashboard 400 of the Data Command center 001 of, for example, FIG. 1, can be auto-populated via an electronic dataflow established between the Data Command center 001 and one or more computer systems of servers that comprise patient information (e.g., such as electronic medical records). The dataflow to and from the medical records dashboard 400 can comprise a two-way flow from the source of patient data to the Data Command center 001, and from the Data Command center 001 to the data source.

In some embodiments, the medical records dashboard 400 can include visual cues, icons, or markers representing and/or enabling access to detailed information related to medical services, procedures or tests provided to the patient. Further, by employing data visualization techniques, a user's eye can be trained to quickly identify these icons or markers and increase the efficiency of user accessing key medical indicators such as test results and surgical histories. For example, in some embodiments, medical services, procedures or tests performed or provided can be assigned a visual code, icon, or graphical marker. For example, the embodiment of the medical records dashboard 400 of FIG. 4B depicts visual cues, icons, or markers 885 representing medical services, procedures or tests performed or provided to a patient. In some embodiments, the information columns 800 within the display window 500 can include at least one “test done, no image attached” icon 885a, one or more “see image in order viewer” icon 885b, at least one “view order interpretation” icon 885c, and/or at least one “procedure billed or claims made” icon 885d, where an appearance in the medical records dashboard 400 can indicate that a claim was made, and a change in color or other method (italics, bold, etc.) can represent whether the bill was paid. Further, FIG. 4D depicts another example of “test done, no image attached” icon 885a, “see image in order viewer” icon 885b, “view order interpretation” icon 885c, and “procedure billed or claims made” icon 885d, in which an appearance in the medical records dashboard 400 represents a claim was made, and a change in color or other notification method can represent whether the bill was paid. Data visualization icons and markers located in the medical records dashboard 400 can be used to quickly identify billing or coding errors by enabling a user to determine inconsistencies among various entries in the medical records dashboard 400, and thus can empower the physician to be proactive and thorough in areas of compliance with insurance guidelines. The use of these icons to identify potential errors in coding can provide an additional level of protection and proofing to reduce and prevent potential billing and/or malpractice errors.

In some embodiments, the medical records dashboard 400 can provide a text summary of any entry within the medical records dashboard 400. As described earlier, the summary window 475 can enable a user to view and edit summary information related to the patient, any details of care provided to the patient, and/or and any medical diagnosis information prepared by a medical practitioner. In some embodiments, the user can add and/or edit the summary information. For example, FIG. 5A depicts the medical records dashboard 400 including a medical summary update process in accordance with some embodiments of the present principles. In the embodiment depicted in FIG. 5A, the medical records dashboard 400, including the problems window 425, surgeries window 450, summary window 475, and command center visual display window 500, can further include summary comments 482 that can be entered, updated, expanded using the summary input window 484. In some embodiments, a user can enter information within the summary input window 484 for entry into the summary window 475.

FIG. 5B depicts a portion of the medical records dashboard 400 including a notes update procedure in accordance with an embodiment of the present principles. For example, using a notes update procedure, a user can add or update information associated with any of the user-accessible tests, procedures or services or other notes provided by the user and/or medical care provider in the notes column 1300. As depicted in the embodiment of FIG. 5B, the medical records dashboard 400 can include the problems window 425, the surgeries window 450, and the summary window 475, and the visual display window 500 of with notes column 1300 of the medical records dashboard 400 can be updated with one or more notes using the note entry window 1305.

In some embodiments, placement or viewing functions of the medical records dashboard 400 can be toggled using a left or right mouse click function. For example, in some embodiments, following an initial impression or diagnosis, a right click can bring up a note function (e.g., through note entry window 1305), and/or a left click can bring up the summary function (e.g., through summary window 475 as summary comments 482).

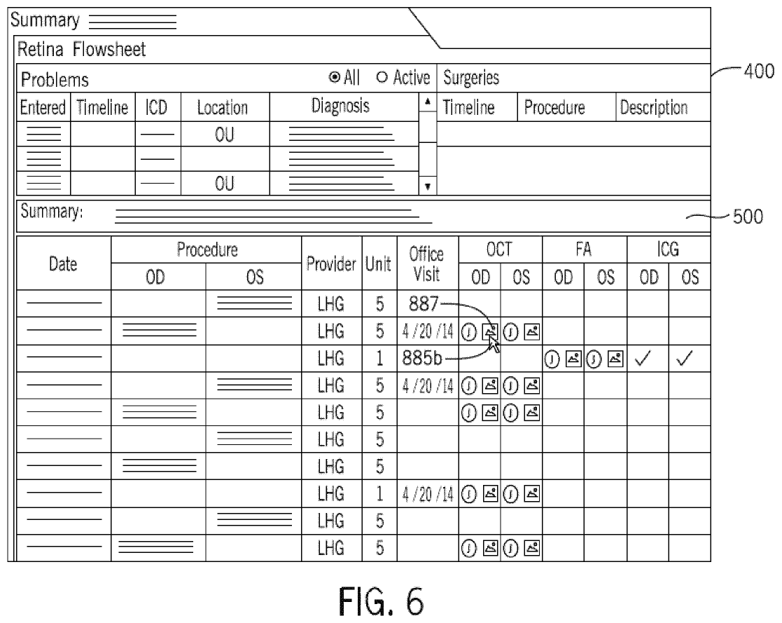

Referring to at least FIG. 4B, in some embodiments, a user can access underlying information linked to visual cues, icons, or markers 885 by, for example, using a single click or mouse-over. That is, in accordance with the present principles, in some embodiments a user can use the visual display window 500 of the medical records dashboard 400 to access and view any information auto-populated within the visual display window 500 and/or other windows or sub-windows of the medical records dashboard 400. For example, FIG. 6 depicts a user record access process of the medical records dashboard 400 in accordance with an embodiment of the present principles. In some embodiments, a user action 887 (depicting a user click or mouse-over of a cursor) can enable a user to access and view information (in this example, information linked to “see image in order viewer” icon 885b). In some further embodiments, a user can use a single click of or mouse-over of a portion of the medical records dashboard 400 to access and view any information within any portion of the medical records dashboard 400. Further, in some embodiments, a user can use left and right mouse clicks to navigate from one portion of the medical records dashboard 400 to another. Furthermore, in some embodiments, a right-click mouse function can be used to bring up and update a an portion of the medical records dashboard 400 and/or display any important information in the medical record dashboard 400, and a left-click can bring the user back to another portion of the medical records dashboard 400.

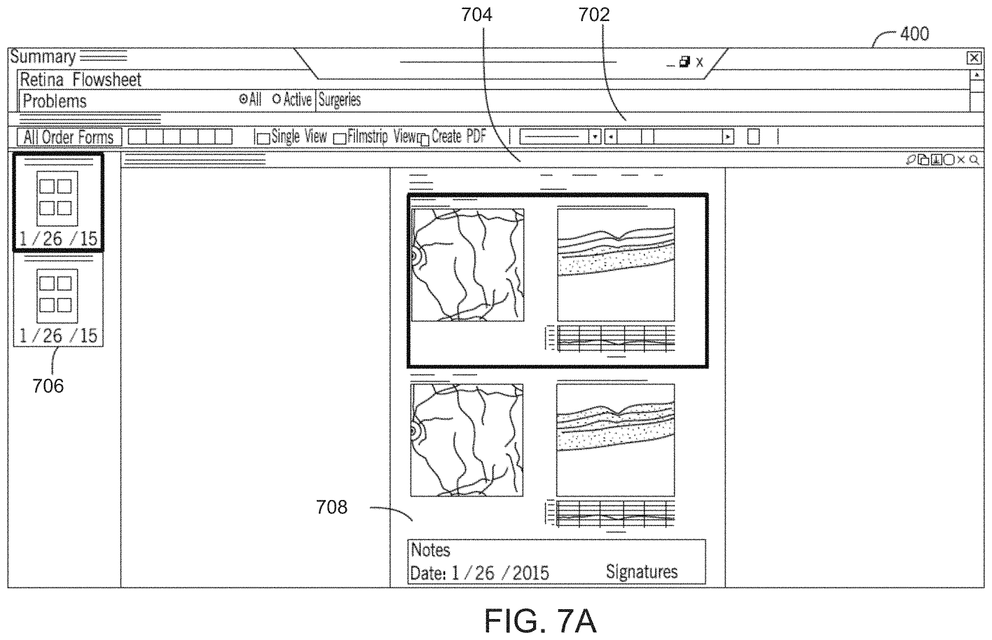

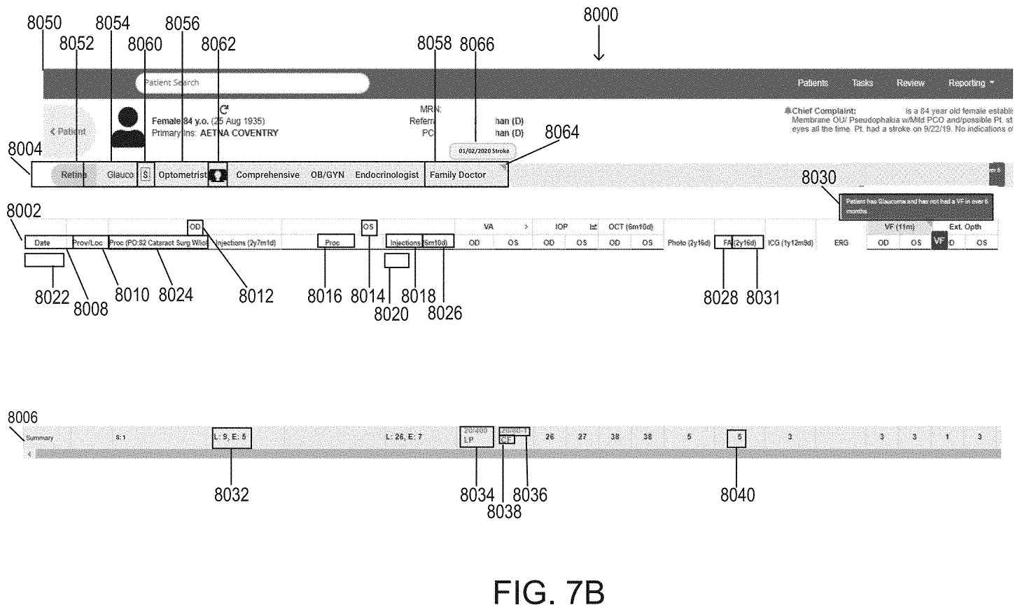

In some embodiments, the Data Command center 001 via the medical records dashboard 400 can display at least one medical record as a result of the user action 887. For example, FIG. 7A depicts a medical records access window 702 of the medical records dashboard 400 in accordance with an embodiment of the present principles. In some embodiments, the user's action (represented by user action 887) can cause a display of the medical record access window 702 including a medical record display 704. Further, in some embodiments, at least one medical record 708 can be selected from the medical record list 706 for viewing in the medical record display 704. As illustrated in FIG. 7A, in some embodiments, the at least one medical record 708 can comprise an image or photograph such as an optical and/or fluorescein angiogram image. In other embodiments, the at least one medical record 708 can comprise an X-ray image. In some further embodiments, the at least one medical record 708 can include an MRI scan or any report or anything ordered or performed by medical care providers. In some embodiments, the at least one medical record 708 can comprise one or more dictated letters from the user or another medical care provider. Further, in some embodiments, the at least one medical record 708 can comprise a record or any portion of a correspondence from another medical care provider.

A unique aspect of the medical records dashboard 400 of the Data Command center 001 in accordance with the present principles is that so much relevant patient information can be viewed in context to the procedures, the clinical information and/or the medical services provided overtime while having direct one click access to any image and diagnostic test or plan. In addition, in embodiments of the present principles all of the patient studies can be accessed in context of all other patient data.

In some embodiments, images related to patient treatment can be viewed as thumbnails in one or more windows being visible and accessible along with at least portions of all other data available on the medical records dashboard 400. In some embodiments, a user is able to manipulate and modify an image with the ability to store and recall the modified image. For example, a user, while looking at an image in context, can mark and make notations, draw on the image. Such modifications can be stored with the image or with a copy of the image.

In some embodiments, thumbnails of respective images can be displayed with an ability to see the full-size image including relevant information. For example, in some embodiments, thumbnails can be displayed across the bottom of a display of all images in a column of the medical records dashboard 400, one per image, such that a user is able to pull up all visual fields of a column, such as the OCT column. Further embodiments describing the display of patient care related images are described further below. In accordance with the present principles, what is critical is not that the Data Command center 001 via the medical records dashboard 400 can display images related to patient care, but instead that all of the images can be looked at in context with clinical and exam findings and or procedural information and dates of other medical services lined up in an intuitive way that enables a user to quickly decide which image or test to review, and in some embodiments, receive guidance from the Data Command center 001 on how to proceed with treatment using various tools and functionality of the Data Command center 001 described herein.

In some embodiments, a user is able to assign a respective icon for accessing and representing images in the medical records dashboard 400. In some such embodiments, the assigned icon can visually represent information related to the image, including whether or not the image indicates that a patient's condition has gotten better, worse or has remained the same.

In some embodiments, the Data Command center 001 via the medical records dashboard 400 can enable a user to access underlying information linked or related to diagnostic codes listed in the medical records dashboard 400. In some embodiments the Data Command center 001 via the medical records dashboard 400 can enable a user to access underlying information linked or related to billing codes. For example, in some embodiments, using a single click or mouse-over, a user can use information made available via the visual display window 500 of the medical records dashboard 400 to access and view any information related to diagnostic and/or billing codes. In some embodiments, the diagnostic and/or billing code information and payment history can be displayed in a separate document or window. In some other embodiments, diagnostic and/or billing code information can be display overlaid onto the medical records dashboard 400 (e.g., as a pop-up window or transient text and/or graphics).

FIG. 7B depicts the functionality of three specific rows in the user interface in accordance with an embodiment of the present principles. That is, FIG. 7B illustrates how three rows or panels in the user interface 8000 can convey a plethora of information for a healthcare professional in some embodiments. Header 8002 is an example of a header that can appear on each individualized specialty-based provider's actionable dashboard. As illustrated at 8004, different actionable dashboards that have been particularly designed for different providers of different specialties can be accessed. In the embodiment of FIG. 7B, a summary row 8006 can be provided on each individualized dashboard for each doctor, specialist, or other user and can be specialized for each user.

In the header 8002, the date is represented at 8008. The date can include a time, day, and/or date of a patient visit or the visit of a group of patients. 8010 can include the initials or name of the provider who cared for the patient or if just a location of testing, can include an indication of the location of the test performed. That is, in some embodiments, a medical care provider's initials can be presented at 8010, which can also include a location, as providers can have multiple offices. In some embodiments, the information in 8010 can include an abbreviation, description, or identifying factor of which office a patient visited. In FIG. 7B 8022 shows an example of one of many patient encounters overtime.

In FIG. 7B, 8012 depicts an example of a column that includes a procedure and, in the embodiment of FIG. 7B, is divided into two different sides of a patient's body. In 8012 OD is displayed, which in eye care refers to a patient's right eye. In the case of orthopedics, 8012 could reference a patient's right knee. Similarly, an OS in 8014 represents the left column or in the case of eye care, the left eye, and in the case of orthopedics can refer to a left knee. As illustrated, the OS or left column of the header can be a procedure 8016. Under the column of the left eye is listed, by encounter, identifying procedures such as injections 8018. Item 8020 depicts a focal procedure that has been performed (e.g., injection of Eylea), while 8022 shows the date of service being Mar. 21, 2017.

As illustrated at 8024, the header 8002 is able to display that a cataract surgery has been performed and that a postoperative period is counting down. The header 8002 can also display that injections 8026 were last performed six months and ten days ago. FIG. 7B depicts that another data element 8028, such as FA, can be displayed over time. In addition, 8030 can display a last time this test or item was performed on a patient. In the embodiment of FIG. 7B, 8030 depicts that a test was performed 2 years and 16 days ago. Header 8002 also includes a pop-up 8030 of underlying information. In the embodiment of FIG. 7B, the patient has a diagnosis of glaucoma and has not had a visual field in over six months.

The summary row 8006 of FIG. 7B shows how not only is the total number of something that had occurred in the rows above counted, but it can be divided according to what was performed. That is, in the embodiment of FIG. 7B, summary row 8006 is a smart summary column. In the embodiment of FIG. 7B, 8032 demonstrates that there were twenty-six Ls and seven Es of which one E (Eylea) is shown at 8020. The embodiment of FIG. 7B depicts an example of a retina doctor who performs injections in the right eye in this case, and used “L,” which stands for Lucentis times and “E,” which stands for Eylea. 8034 similarly demonstrates the summary cell in a column of the left eye. 8036 shows the vision in the left eye is 20/80-1 and could also reflect the best number or event that occurred in the entire row overall of dates of service, and can be highlighted to inform the user that it is the best value. 8038 shows CF. In this illustration of a retina surgeon, CF means count fingers, which is very bad vision and is, therefore, red. For the first time in this illustration, a retina surgeon can know, over the time that the doctor has been delivering services, or any doctor, what is being reflected in encounters and rows above. The best vision was 20/80 (8036). The worst was count fingers (8038). This can also be the best blood pressure and the worst blood pressure. Every different specialty in medicine has different ways that it would like to measure the highs and lows in a column. 8040 simply shows the counting of the number that had occurred in all of the encounters. In this case five FAs 8028.

It is important to note that the tool can measure anything in the row and display it in multiple different ways. The choice could be just to see the high and low as in 8036 and 8038 over a short period of one year or over as many years as there have been encounters. It can also be set to show percentage changes overtime. In any case, this summary provides a tremendous amount of information to the provider for enabling rapid decisions.

A panel 8050 may be located at the top, side, or bottom of the display in FIG. 7B and may provide access for each specialist to different types of healthcare providers or different doctors who want to customize the display. Any type of doctor or dentist or other health care provider of any specialty can be listed. As few or as many as have actionable dashboards that can be accessed immediately with direct access by simply clicking on the specialist's name. For instance, the specialists may be retina doctors 8052, glaucoma doctors 8054, or an optometrist 8056. All three happen to be types of eye doctors. All three could be in the same practice, separate practices, or even in different countries. Each, when clicked on, pulls up an actionable dashboard specially designed for them or their practice in that specialty. 8058 provides an example of a non-eye doctor, in this case, the family doctor.

It is important to note that any health care provider, if given permission by the patient, and each specialty noted in FIG. 7B could see the actionable dashboard of the other specialists for as little or as much as each would allow. There is some information in an actionable dashboard to each specialist, practice, or doctor that they might not want others to see, which can be hidden (e.g., payments and costs). In addition, next to each actionable dashboard can also be additional information that can also be pulled up instead of the actionable dashboard itself. For instance, a dollar sign 8060 could be for providing for each practice or actionable dashboard, payments, costs or any financial matter that can pop-up to show a different type of financial dashboard. 8062 shows an example that can pull up any type of additional information, such as a shared care dashboard between different providers.

8064 illustrates that an entire cell can alert all of the other providers of something important. It can be a color change, or flash, or blink. When activated, it represents that there is some type of important event, for instance, that all providers should know. A pop-up 8066 also may be shown at all times or by hovering over 8064. The popup could represent whatever the important item is to be alerted. For instance, a new diagnosis like the patient had a stroke on 1/2/20, which all providers would like to know. It can also inform all providers that the patient missed an appointment that was very important with that doctor. So, that all specialist would know that and be able to remind the patient.

It will be further appreciated that the actionable dashboard may further include a communication center where users can send messages to each other in a HIPAA compliant way. In other words, a physician, while seeing a patient, can send a message to their chief technician or the office manager to talk about following up on a patient or also to the billing office that there is a billing problem. Then, staff can report back to the doctor and this message can be imbedded into the smart actual dashboard so that the next time a doctor sees the patient through icons and columns of correspondence of communication within the practice, the doctor can pull up what was the response to a message they had sent earlier. This response can be read live while treating the patient so that the doctor can take it into perspective while making decisions. The messaging system, attachments or anything else can be sent to the doctor or health care provider in any way that they would want. Whether through email or the internal messaging system or as a tickler system within the EMR system that automatically toggles back and forth to the actionable dashboard, so the doctors can see their messages at the end of the day or the end of the week, or while seeing the patient. It really helps organize the doctor's life, so this actionable dashboard becomes the communication hub, the switchboard, for the entire practice, while communicating with the health care provider.

With reference to FIG. 7A, in some embodiments, at least one medical record 708 can comprise a transition of care document (hereinafter “CCD”). In some embodiments, the Data Command center 001 via the medical records dashboard 400 can be configured to receive one or more CCDs from one or more medical care providers for display to the user. In some embodiments, the Data Command center 001 via the medical records dashboard 400 can be configured to extract information from the CCD for display to the user. For example, in some embodiments, information from a received CCD can be extracted and used to populate one or more data columns or fields of the medical records dashboard 400 and/or one or more linked data columns or fields of the medical records dashboard 400. In some embodiments, the Data Command center 001 via the medical records dashboard 400 can be configured to receive direct messaging information exchange with other healthcare organizations such as IHE profiles, CDA and CCD, NwHIN Direct, HL7v2, HL7v3, DICOM, X12, ITK (UK), DMP (France), and NEHTA (Australia), etc. For example, in some embodiments, the integration module 002 of the Data Command center 001 can include an HL7 message router and schemas for exchange of direct messages including a graphical editor for transforming messages and data.

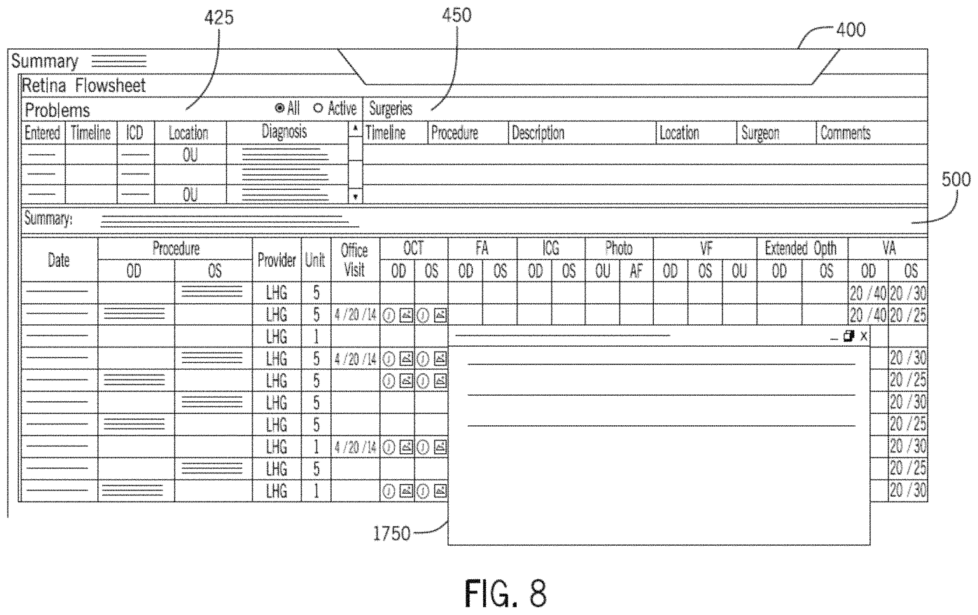

In some embodiments of the present principles, a user via the medical records dashboard 400 of the Data Command center 001 can retrieve and/or update information related to a medical diagnosis. For example, FIG. 8 depicts a medical records and diagnosis update process of the medical records dashboard in accordance with an embodiment of the present principles. In some embodiments, the medical records dashboard 400 including problems window 425, surgeries window 450, summary window 475, and command center visual display window 500 can include an option to enable a user to update or enter at least one medical diagnosis using a medical record/diagnosis window 1450. In some embodiments, multiple medical diagnoses can be provided or updated by a user. In some embodiments, the user providing the medical diagnosis can be any medical practitioner providing the service or procedure to the patient. In some other embodiments, the medical record/diagnosis window 1450 can be updated by a user other than the medical practitioner providing the service or procedure to the patient.

Further, in some embodiments, information can also be auto-populated into the EMR plan pages. A Data Command Center in accordance with the present principles via the medical records dashboard 400 can auto-populate various data fields related to information in any one of the problems window 425, surgeries window 450, summary window 475, and record/diagnosis window 1450 via an electronic dataflow established between the Data Command Center and one or more computer systems of servers that comprise patient information (e.g., such as electronic medical records). The dataflow can comprise a two-way flow from the source of patient data to the Data Command Center, and from the Data Command Center to the source.

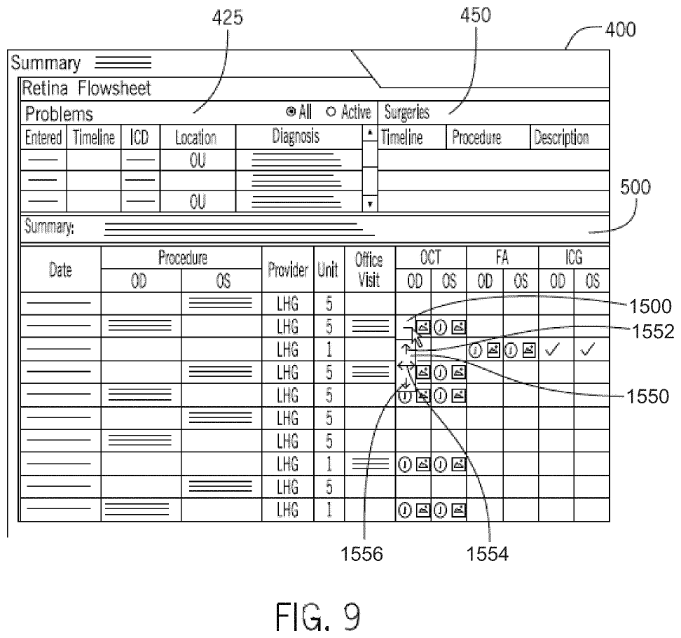

In some embodiments, the Data Command center 001 via the medical records dashboard 400 can enable a user to update information displayed in the visual display window 500. For example, in some embodiments, a user can update information related to a medical diagnosis and/or information related to a medical test or other service or procedure. For example, FIG. 9 depicts a medical record update marker process of the medical records dashboard in accordance with an embodiment of the present principles. That is, in FIG. 9 the medical records dashboard 400, including problems window 425, surgeries window 450, and summary window 475 is depicted with a record update marker 1500 being accessed by a user and displaying an update marker selection tab 1550. In some embodiments, the update marker selection tab 1550 can include a user-selectable marker or icon. For example, in some embodiments, update marker selection tab 1550 can include a selectable diagnosis indicator 1552, a selectable diagnosis indicator 1554, and/or a selectable diagnosis indicator 1556. In some embodiments, the selectable diagnosis indicators 1552, 1554, 1556 can provide a graphical representation of a medical diagnosis, outcome, or test. For example, in some embodiments, the diagnosis indicators 1552, 1554, 1556 can provide a visual representation of an improvement of a medical problem, disease, or symptom, or a worsening of a medical problem, disease, or symptom. Further, in some embodiments, the diagnosis indicators 1552, 1554, 1556 can provide a visual representation of a medical problem, disease, or symptom that is stable or substantially unchanged. In some embodiments, the diagnosis indicators 1552, 1554, 1556 can provide a visual representation directly related to one or more variables of a physical test. For example, in the field of ophthalmology, some imaging tests can provide an analysis of the thickness of the retina related to an eye disease such as macular degeneration. In some embodiments, an increase in thickness can represent a worsening of the condition, whereas a decrease in thickness can represent an improvement. A stable or unchanged thickness can indicate the disease is responding to treatment or is in remission. Further, by using data visualization techniques such as by using a color change or other method (e.g., such as using italics, bold text, and/or underlined text), a particular important change in a test can be marked for internal reference alerting a physician to the tests or procedures that are important and to take note for future reference. Further, in some embodiments, the diagnosis indicators 1552, 1554, 1556 can comprise a color and/or graphical change providing a visual representation of items billed, items not billed, or tests needing reports or interpretations are required. A color change or data visualization method (e.g., such as using italics, bold text, and/or underlined text) can also tell a physician if a test or procedure was billed, rejected, or if an interpretation needs to be made.

As an example embodiment, the diagnosis indicators 1552, 1554, 1556 can provide a visual representation of the status of a patient with an eye disease such as macular degeneration. For example, in some embodiments, the diagnosis indicators 1552, 1554, 1556 can be selected from the update marker selection tab 1550 when the user intends to indicate a worsening of the condition (e.g., where the thickness of the retina is increasing). In some embodiments, any of the diagnosis indicators 1552, 1554, 1556 can be color-coded to represent a status or provide a visual indicator of a medical condition, test, or diagnosis linked to the diagnosis indicators 1550. For example, in some embodiments, the diagnosis indicator 1552 can be color coded red and the diagnosis indicator 1556 can be color-coded green. Further, the diagnosis indicator 1554 can be color-coded blue or black. In some other embodiments, the diagnosis indicator 1552 can be color coded green and the diagnosis indicator 1556 can be color-coded red. In other embodiments, other graphical markers or icons can be used, and/or other colors can be used to differentiate the diagnosis indicators 1552, 1554, 1556. Further, in some embodiments, in addition to or in place of using a color differentiation between the diagnosis indicators 1552, 1554, 1556, one or more of the diagnosis indicators 1552, 1554, 1556 can flash or pulsate.

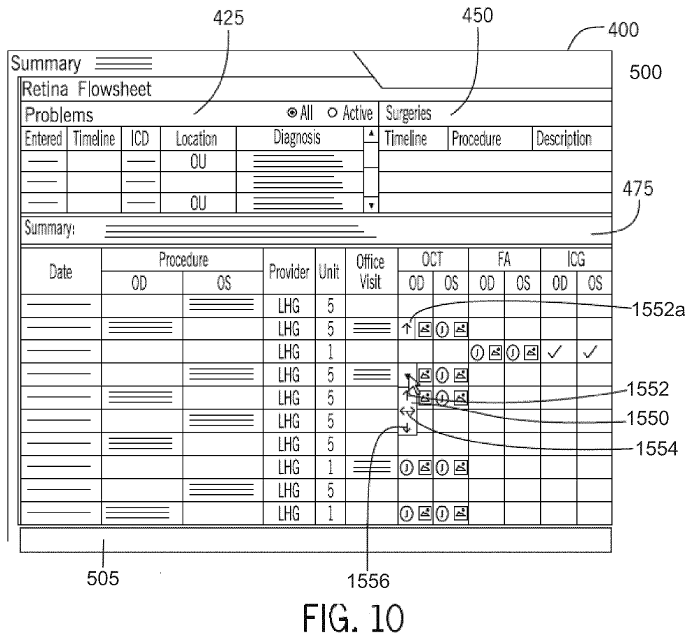

In some embodiments, the Data Command center 001 via a medical records dashboard of the present principles, such as the medical records dashboard 400, can enable a user to provide a plurality of updates to information displayed in the visual display window 500. For example, in some embodiments, a user can update information related to a medical diagnosis and/or information related to a medical test or other service or procedure, and subsequently provide further updates to the same information or to other information. For example, FIG. 10 depicts a medical record update marker process of the medical records dashboard 400 in accordance with an embodiment of the present principles. The embodiment of the medical records dashboard 400 of FIG. 10 includes a problems window 425, surgeries window 450, summary window 475, and command center visual display window 500. The command center visual display window 500 depicts diagnosis indicator 1552a representing previously updated information. The visual display window 500 also illustrates a user updating information with a process described above using the update marker selection tab 1550 comprising a selection of diagnosis indicator 1552, diagnosis indicator 1554, or diagnosis indicator 1556. In some embodiments, the diagnosis indicator 1156 can be modified to be indicative of updated information or status of a patient and/or a patient's disease, test, or medical condition. Further, any ICD, SNOMED or similar code can be inserted.

In addition, FIG. 10 illustrates a portion of the medical records dashboard 400 including a scrolled display in accordance with some embodiments. In some embodiments, the medical records dashboard 400 including problems window 425, surgeries window 450, summary window 475 can include a command center visual display window 500 that comprises a scroll display 505. In some embodiments, any information displayed in the command center visual display window 500 can be scrolled by the user to bring non-visible portions of the command center visual display window 500 into view. Such capability can enable the user to view the entire history of the patient independent of the number of years of history that is on record.

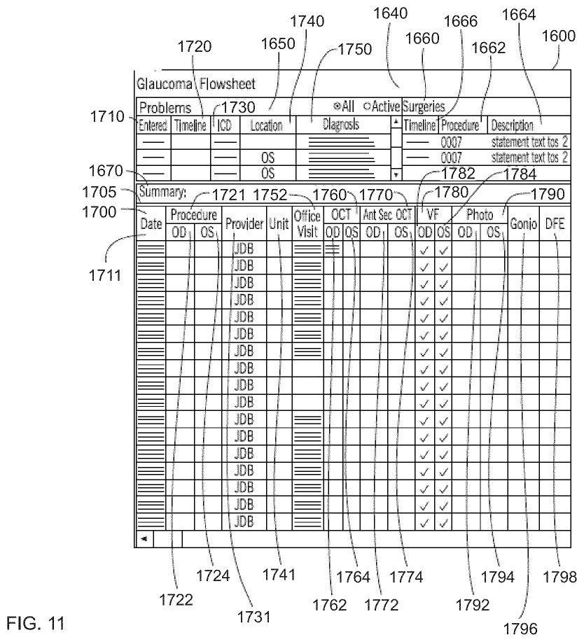

FIG. 11 depicts a portion of a medical records dashboard 1600 in accordance with another embodiment of the present principles. In some embodiments, the medical records dashboard 1600 can display data from one or more medical records, and/or track medical procedures and services based on claims made or billing signed off by a physician for one or more delivered medical procedures or services. Further, in some embodiments, the medical records dashboard 1600 can be auto-populated as a function of claims made or billing signed off by a physician, auto-populated from any portion of a selected chart. In this instance, any data displayed within the medical records dashboard 1600 can be derived from one or more claim records that have been billed for one or more procedures or services have previously been provided to the patient. In reference to the medical records dashboard 1600 and/or the previously described medical records dashboard 400, in some embodiments, auto-populating visits by actual claims made or billings signed off by a physician, by definition occurs after the visit with a patient.

In some embodiments, the Data Command Center of the present principles can auto-populate some information of the medical records dashboard at the time the patient is seen, or shortly thereafter, or even before in preparation for a visit (i.e., lab results), so that even if a patient is not seen on a particular day, the user (e.g., medical care provider) can view the displayed information in the table for information. For example, in some embodiments, information related to vision can be made with the current date at the time patient is seen. In some embodiments, a user or user's assistant can update the Data Command Center with medical tests or test results (e.g., a vision test) as they are performed or shortly thereafter (i.e., on the same day). In this example, this information can immediately trigger the current date and auto-populate the vision column. This information can then be immediately viewed by a user and/or medical care provider and can be updated with notes or comments or other information as the user and/or medical care provider is attending to the patient. Further, after the claim has been made for any diagnostic tests or examinations or procedures that have not yet been billed, the date will then auto-populate in the future with the other related columns. In some embodiments, while examining a patient, important information and/or certain parameters that are critical to follow can be immediately updated to the Data Command Center. Using these procedures, the Data Command Center of the present principles can enable a medical care provider to review the patient's medical history, treatment history, and instantly see items of importance on the day they're examining a patient. For example, the user and/or medical care provider can be enabled by the Data Command Center, on the day the patient is examined, to review information such as a vision or glaucoma table, intraocular pressure, blood pressure, blood sugar, etc. When billing claims are made, further information is filled to complete the billed claims record. As a further example, a patient may be seen a few days apart and the diagnostic tests etc. and claims have not yet been made, however the Data Command Center can be configured to show that the patient was seen that day (e.g., with a vision, pressure test, etc.), and the Data Command Center can enable a user (such as a physician) to interpret and/or add special notes on the day they see a patient or before they see the patient rather than waiting to make some notes when a claim is actually generated.

If a medical office wishes to communicate results or a test (e.g., a pathology result or test) to a user, in some embodiments, a blinking cursor can appear to alert the user. Also any written or typed correspondence or any links to dictated information using voice recognition can be coupled to or integrated with a medical records dashboard via the Data Command Center of the present principles. For example, in some embodiments, the integration module 002 of the Data Command center 001 of FIG. 1 can integrate such information into the medical records dashboard. In some embodiments, information can be auto-populated into the medical records dashboard with the touch of a button into a designated location such as the current plan documenting the patient's current visit (thus aiding documentation for the current visit). Further, whatever is important fora user to input into the day's visits for documentation can be initially inputted in the table, and then permanently into the day's patient visits. Further, a summary section of a medical records dashboard can be constantly fluid, and can be changed at every visit rather than being written to an unchangeable document or file (e.g., such as a PDF). Any patient data that is inputted, received, analyzed, or created can be auto-populated into any portion of a medical records dashboard. The Data Command Center can auto-populate in a one-way or two-way direction in various data fields related to information in any patient information via an electronic dataflow established between the Data Command Center and one or more computer systems of servers that comprise patient information (e.g., such as electronic medical records). The dataflow can comprise a two-way flow from the source of patient data to the Data Command Center, and from the Data Command Center to the source including another electronic system or server, or another user, observer, or other 3rd party.

By following a patient on the day of delivery (e.g., for a vision intraocular pressure or anything else) the Data Command Center can enable the user and/or medical care provider via the medical records dashboard to see the diagnostic test on same day even though it has not been billed. Further, this procedure can enable the medical care provider to optionally add a note and allow free hand typing at the end of the line.

In some embodiments, medical information populated within medical records dashboard (e.g., shown as visual cues, icons, or markers 885 representing medical services, procedures or tests performed or provided to the patient) can include a visual marker such as a red dot. In some embodiments, the Data Command Center can display the red dot until a claim is actually made at which time the Data Command Center can display a green dot (i.e., the Data Command Center can convert the red dot to a green dot). In some embodiments, by clicking on the dot, the user can toggle between the payment screen and the command center visual display window 500, 1700. This can allow medical care providers to improve patient care, to review the actual picture of a diagnostic test that is displayed within the command center visual display window 500, 1700, to review other diagnostic tests results, and to compare to what happened on other days. In some embodiments, at any time, a medical care provider can click on the dot to access a display where the claim is billed, and any payment that was made can be displayed. This process can help to reduce medical errors enabling medical care providers to quickly review the billings and claims made or billings signed off by a physician and payment portions of the Data Command Center. Further, this procedure serves as an additional tool to minimize coding, compliance with insurance guidelines, and medical treatment errors, as the Data Command Center can provide a quick reference tool that can pull all critical medical and procedure data from the patients EMR chart into a concise and clear table.

In some embodiments, a medical records dashboard of the present principles can display information related to medical procedures or services in relation to care of a patient with glaucoma. In some embodiments, the medical records dashboard can display various windows and sub-windows based on a user preference and/or current or previous user interaction with the medical records dashboard. As depicted in FIG. 11, in some embodiments a medical records dashboard 1600 can include information columns 1640 including a problems window 1650 and/or a surgeries window 1660 where information related to a patient's medical problems and surgeries can be displayed. In some embodiments, the medical records dashboard 1600 can include a summary window 1670 enabling a user to view and edit summary information related to the patient, any details of care provided to the patient, and/or and any medical diagnosis information prepared by a medical practitioner. Further, the medical records dashboard 1600 can also display detailed information related to any medical procedures or services provided to the patient, including procedures or services that are auto-populated by claims made or billing signed off by a physician as detailed above or other method. For example, in some embodiments, the medical records dashboard 1600 can display a visual display window 1700 including a plurality of information columns 1705. In some embodiments, the visual display window 1700 can be scrolled by the user to display other portions of the visual display window 500.

In some embodiments, the medical records dashboards of the present principles can also display detailed information related to notification of payment of any medical procedures or services provided to the patient, including procedures or services that are auto-populated by claims made or billing signed off by a physician as detailed above or other method. Moreover, the medical records dashboards can enable a user to access and/or track the status of the billing and payment process at any point in time. For example, in some embodiments, the medical records dashboards can access and view any patient encounter form (i.e. a superbill), any claims made to a clearing house, any updates on accepted or rejected bills from the clearing house, any claims made to an insurance company, and/or any payments received for any claims made.

As depicted in FIG. 11, in some embodiments of a medical records dashboard, the problems window 1650 can include a date and time information in entered date column 1711, a timeline column 1720, an “ICD” column 1730 for ICD code information, location of the problem or disorder (shown as “OD”, “OS”, “OU” identifying right eye, left eye, both eyes) (column 1740), and a diagnosis column 1750 for detailing information related to an initial diagnosis or final diagnosis of a patients problem or disorder. Further, the surgeries window 1660 can include information related to services or procedures were provided to the patient (procedure columns 1662), a description of the services or procedures performed (description columns 1664), and when the services or procedures were provided to the patient (shown as timeline columns 1666), and can include a surgical report that can be brought up and viewed by the user.

Referring to the visual display window 1700 of the medical records dashboard 1600 of FIG. 11, the information columns 1705 can include a date column 1711, and a procedure column 1721 illustrating or providing access to information detailing one or more procedures performed on the patient. Further, the procedure column 1721 can include an “OD” column 1722, and “OS” column 1724 providing right and left eye procedure information. In some embodiments, information related to the medical care provider, location where the procedure was performed, and office visit information can be provided to the user in the provider column 1731, and unit column 1741, and office visit column 1752. In some embodiments, the visual display window 1700 can enable a user to view information related to tests and procedures performed on the patient including, but not limited to one or more medical imaging procedures such as an optical coherence tomography (“OCT”), or fluorescein angiography (“FA”), and/or indocyanine green chorioangiography (“ICG”). In some embodiments, medical procedures performed (including any of the aforementioned medical imaging procedures) that have been billed and claimed can be viewed or accessed by a user within any of the “OCT” column 1760 (shown split as an “OD” column 1762 and “OS” column 1764), an “Ant Seg OCT” column 1770 (split as an “OD” column 1772 and “OS” column 1774).

In some embodiments, if visual function tests were performed, information can be viewed or accessed in the “VF” column 1780 (including an “OD” column 1782, and/or an “OS” column 1784. Some embodiments include a photo column 1790 configured to enable a user to access any photographic images of the patient's eyes including optical and/or auto-fluorescent images of the eyes (“OD” column 1792 and “OS” column 1794). Further, the embodiment of the medical records dashboard 1600 of FIG. 11 includes a Gonio column 1796 providing access to gonioscopy data and/or information related to a dilated fundus examination (“DFE” column 1798). In some embodiments, the surgeries window 1660, can include a location column, a surgeon column, and a comments column (not shown).

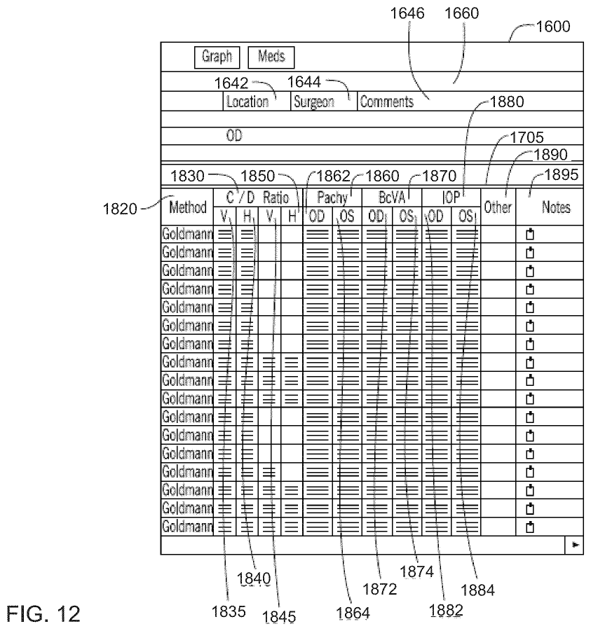

In some embodiments, the visual display window can enable a user to view information related to tests and procedures performed on the patient including a cup-to-disc ratio (“C/D”) to assess the progression of glaucoma, Pachymetry data (“Pachy”), refraction test information such as best-corrected visual acuity (“BCVA”), and/or intraocular pressure (IOP) data. For example, FIG. 12 depicts a portion of the medical records dashboard 1600 of FIG. 11 including column 1820, “C/D ratio” column 1830, “Pachy” columns 1860, “BcVA” columns 1870, and “IOP” columns 1880. In some embodiments, the “C/D ratio” column 1830 includes “V” column 1835, “H” column 1840, “V” column 1850, and “H” column 1850. Further, in some embodiments, the “Pachy” columns 1860 includes “OD” column 1862, and “OS” column 1864. In some embodiments, the “BcVA” columns 1870 includes “OD” columns 1872, and “OS” columns 1874. Some embodiments include “IOP” columns 1880 including “OD” columns 1882, and “OS” columns 1884. In some embodiments, other columns 1890 can be used to add additional test information. Further, the visual display window 1700 can also include a notes column 1895 for accessing and updating notes related to tests and medical diagnosis. In some embodiments, the tracking display window 1700 can be updated with comments and notes as described earlier.

In some embodiments, the Data Command Center can display and auto-populate a medical records dashboard of the present principles, such as the medical records dashboard 400 and/or the medical records dashboard 1600, with more than one patient information. For example, in some embodiments, any windows, sections, or columns of the medical records dashboard can display information related to a plurality of patients. Any patient data that is inputted, received, analyzed, or created can be auto-populated into any portion of the dashboard, where the Data Command Center can auto-populate in either a one-way or a two-way direction. Thus, data fields related to information in any patient information can be communicated via an electronic dataflow established between the Data Command Center and one or more computer systems of servers comprising patient information (e.g., such as electronic medical records). Further, in some embodiments, any information displayed by Data Command Center can display and auto-populate the medical records dashboard as a function of patients seen during a specified time period. In some other embodiments, the Data Command Center can display and auto-populate the medical records dashboard as a function of a specified disease and/or diagnosis. For example, in some embodiments, the Data Command Center can display and auto-populate the medical records dashboard as a function of a diagnosis or procedure or prescribed medication or lab or imaging test from input received from a physician or other medical practitioner or provider. For instance, every patient who has the diagnosis of diabetes with their name and the date last scene is auto-populated. Certain parameters that may need to be followed by the user from all of their patients with this condition can be auto-populated. For example, in the case of patients with diabetes, parameters can include how often they've missed appointments, blood sugar, hemoglobin A1C, medications, major new medical complications such as heart attack, stroke, amputations, blindness, each of which can be auto-populated and followed to enable the user to see how all their patients are doing. In some embodiments, input and the ability to display data can be based on single values or on complex multi-variate input (i.e. patients with diabetes, taking metformin and seen by the practice in the last 30 days).

In some embodiments, a user(s) can receive, via the medical records dashboard, a daily report on all the patients they have seen, what the diagnosis codes are and what CPT, ICD, or office visit billing codes were done whether they have been billed or not. In some embodiments, the user can view a report of patients for a specific day or week based on appointments or other data such as referrals. With this functionality, at the end of the day physicians can see all of their activity or the practice's activity on the medical records dashboard of the present principles and using data visualization techniques can realize what activity still needs to be completed or was not entered properly. The user can then review the same information in a few days' time and weeks or months later to ensure that the proper billing and collections has occurred. Additionally, the medical records dashboard enables direct one click access to underlying data, so without leaving the screen, providers can make medical decisions. For instance, an icon displayed on the medical records dashboard can represent that a test was performed on a particular patient or group of patients and that test can be directly accessed by activating that icon. If more information is needed, the entire individual flowsheet of the patient can be, with no more than one click, brought up, decisions made, and then with one click, return to viewing the entire medical records dashboard.

In some embodiments, two monitors can be implemented during which a first monitor can display the medical records dashboard and the second monitor, controlled by the first, can display the data from a selected patient. In such embodiments, even a single click to return to the medical records dashboard is not needed as information can be displayed on both monitors at once. In some embodiments, a portion or all of the data of a medical records dashboard, as well as the diagnostic tests, can be sent to a patient portal, to an email server, and/or as a fax. Further, in some embodiments, a user can be alerted via the medical records dashboard, when claims are sent out for payment and when claims are actually paid. For example, in some embodiments, the above described methods of display can provide a mechanism for displaying payments to the user, and if claims are being made for each patient seen in any particular day, week or month.

In some embodiments, any report, note, letter, referral or diagnostic test can be sent from a medical records dashboard of the present principles to an EMR, patient portal, a messaging platform to an email server, and/or as a fax. Messages can be transmitted to a patient or another practice focused on appointment reminders, medication prescriptions and/or refills, and good care management guidelines. It should be recognized that data interoperability and messaging are not limited to the examples provided but apply to any information within the Data Command Center.

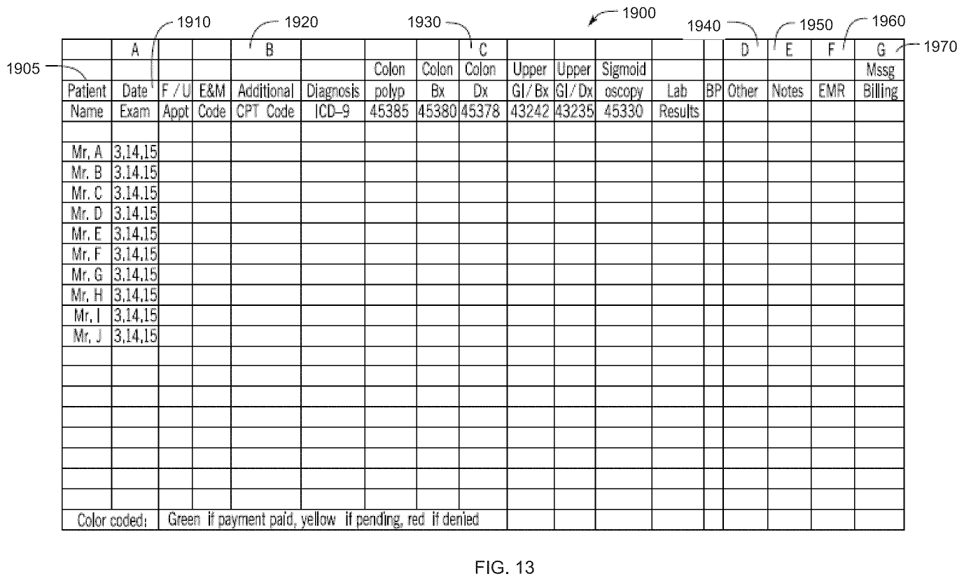

FIG. 13 depicts a portion of a medical records dashboard 1900 configured for display as a function of disease or patient in accordance with another embodiment of the present principles. In some embodiments, the medical records dashboard can be displayed overlaid on a previously viewed dashboard such as the medical records dashboard. For example, in some embodiments, the medical records dashboard can be displayed in the visual display window 500. In other embodiments, the medical records dashboards can be displayed independently from the medical records dashboard, and the user can toggle a display of any of the medical records dashboards.

The medical records dashboard 1900 of FIG. 13 provides a list of patients 1905, and within column 1910, an entire day of patients listed by date or by insurance coverage can be provided or the list can comprise a single patient with multiple visits. For example, in some embodiments, within column 1920 of the medical records dashboard 1900, an office visit and any items billed for a routine examination day and any other CPT codes billed that day can be displayed. In some embodiments, some specialties will have many CPT codes during an office visit (e.g. Ophthalmologists), whereas others (e.g., Gastroenterologists) can have four during an office visit. In some embodiments, column 1930 of the medical records dashboard 1900 can include the procedures that a physician can perform and are usually not on the same day as the exam (these can be GI physician examples). In some further embodiments, the column 1940 of the medical records dashboard 1900 can include various important parameters that can be followed for a specific patient. Some embodiments of a medical records dashboard, such as the medical records dashboard 1900, include column 1950 that enables a physician to write notes about patient care issues and column 1960, which enables a user to access that patient's personal EMR or review table and can also send a message to the patient. In some embodiments, the column 1970 of the medical records dashboard 1900 can enable a user access to the charge payment history of the patient and also enable a message to be sent to the billing department from this table. In some embodiments, columns can be reconfigured such that all patients with a particular insurance company that have a particular diagnosis on any given date or over a prolonged period of time can have tests and procedure results, as well as payments compared, allowing on one screen anyone to rapidly move through this visual display system to enable rapid comparison of results and potential payment anomalies for the same insurance company for the same CPT codes but perhaps different ICD9 or 10 diagnosis and to see if there is a mismatch (e.g., through the use of artificial intelligence systems discovering insurance payment irregularities). In some embodiments, columns 1920, 1930 of the medical records dashboard 1900 can be colored ‘black’ when a claim is made, and can be colored ‘green’ if paid, and can be colored ‘yellow’ if a payment is pending, and can be colored ‘red’ if payment denied by one rendition, e.g., physician reconciliation report of messages sent individuals for follow up, and/or a report of all the message activity from any given day.

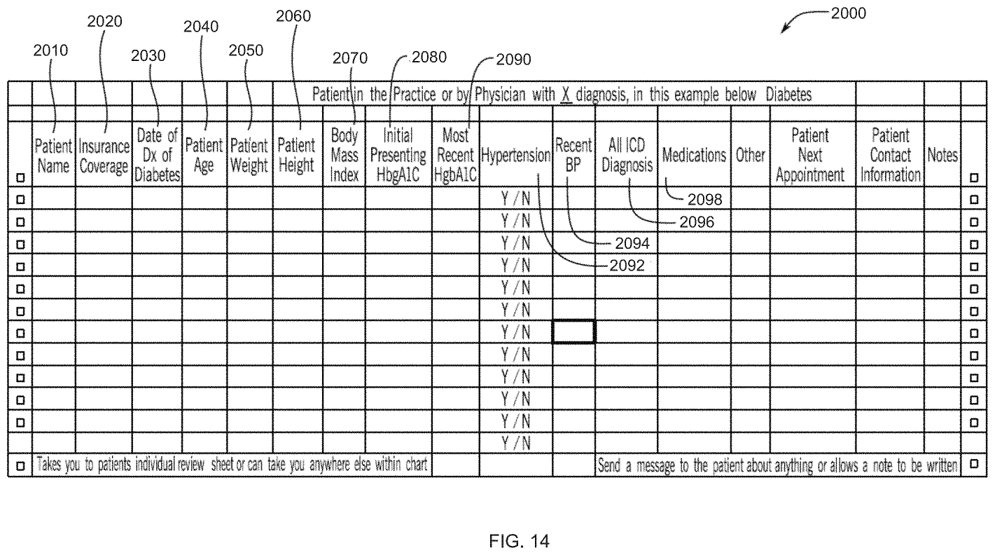

FIG. 14 depicts a portion of a medical records dashboard 2000 configured for display as a function of disease of a patient and specifically configured to display data related to patients with diabetes in accordance with another embodiment of the present principles. In the medical records dashboard 2000, the display for patients 2010 can include a variety of medical, billing, and insurance related information. FIG. 14 illustrates a portion of a medical records dashboard for display as a function of patients with a specific disease ICD, such that many patients may be compared at the same time, which can be useful for clinical research or for tracking clinical outcomes. The embodiment of FIG. 14 tracks all patients in a practice or a subset of patients and can compare, for example, particular diagnostic test results in patients with a particular condition, the results of a particular medication, how all the patients did who received a particular intraocular lens into the eye, etc. The information is put into each individual patient's table, but all patients with that issue can also be called up on a single table such as illustrated in FIG. 14. In some embodiments, such functionality can compile and compare all patients with a particular insurance company that have a particular diagnosis and compare tests and procedure results, as well as payments, allowing a user on one screen to rapidly see results, changes, patterns and payment anomalies. This feature also allows the extraction of patients with similar conditions for referrals and clinical research, batch generation for cross-referrals (e.g. optometry for an ophthalmologist), etc. In some embodiments, the medical records dashboard 2000 can be displayed as shown or can be sorted based on any of the data columns. For example, the patients 2010 can be shown including information displaying insurance coverage 2020, date of diagnosis of diabetes 2030, the patient's age 2040, the patient's weight 2050, the patient's height 2060, the body mass index 2070, the initial presenting HbgA1C 2080, the most recent HbgA1C 2090, the hypertension status 2092, the recent blood pressure 2094, the All ICD diagnosis 2096 and the current or past medications 2098. In some embodiments, the medical records dashboard 2000 can be reconfigured to display patients 2010 sorted by any of the columns 2020, 2030, 2040, 2050, 2060, 2070, 2080, 2090, 2092, 2094, 2096, 2098.

The medical records dashboard 2000 of FIG. 14 enables a user to present on one display everything the physician has done in a particular time frame, such as a day. For instance, a line of information for the information entered into the chart for every patient for today's visit, can be presented in the dashboard 2000. Whatever the user records, like the patient's vision and any diagnostic tests or laser procedures or injections in the eyes that were actually done that day, can be displayed on the medical records dashboard 2000 for interpretation. A medical records dashboard of the present principles can also enable a user to configure follow-up enabling a user to check again (e.g. 48 hours later) when everything for that day should have already been billed or 60 days later when everything is paid and all dates queried can be compared. Also, the user is able to review all patients having a common insurance carrier to facilitate satisfactory payments from the insurance company.

In some embodiments, a Data Command Center of the present principles can enable an addition of a date alert or self-destruction of any information or data entered or auto-populated in the medical records dashboard 400. For example, in some embodiments, any message, or note, or summary, or any medical data can include a date alert and/or a self-destruct function that can remove and/or delete information from the medical records dashboard 400. In other embodiments, the historical date and/or an alert or warning can be provided with any auto-populated or user-summoned information to assist the user with an assignment of relevancy to any data being reviewed prior to, during, or after a patient visit or examination. In some embodiments, this feature can optimize the standard of care being delivered by the user. For instance, this feature can help monitor preferred practice patterns or serve as a reminder on information needed for clinical review.

In some embodiments, a Data Command Center of the present principles enables the prioritization of relevant data in at least portions, columns and rows of a medical records dashboard, while minimizing less important values. This functionality enables a user to focus on the most important data pertinent to the current use case (i.e., with a patient that has a certain diagnosis, several preferred diagnostic test results and data are germane). In some embodiments, such display capabilities can be applied to data that originates from additional users/EHR deemed important and which can be rendered in chronological order. Utilizing Artificial Intelligence (AI), Natural Language Processing (NLP), and/or conventional business logic, a Data Command Center of the present principles can programmatically filter out unnecessary information and queries for display.

In some embodiments, a medical records dashboard of the present principles can be configured based on key events, results, date/time, and/or logical parameters which can include, but are not limited to Diagnoses, Medication Start/End Dates, Allergy Start/End Dates, Billing History, Demographic Data, Observations/Plans, and Life Events. In accordance with the present principles, the format and display of rendered data in the medical records dashboard will make maximum usage of space by shrinking less relevant rows, auto-sizing of columns, and automatically collapsing less relevant data. The intention of this functionality is to provide the most efficient view in the medical records dashboard of relevant data, while not overloading the provider with information not germane to the current configuration. An example would be if a patient has glaucoma, there are specific columns in the medical records dashboard that are highly important to monitoring the chronic condition but some which have no relevance. In this example, the patient that has a diagnosis of glaucoma will have Intra-Ocular Pressure (IOP), Glaucoma medications, etc. displayed prominently while the other less relevant columns are masked.

In some embodiments, the Rules module 004 of the Data Command Center of the present principles can include a Flowsheet Editor Interface that provides a method by which a user/medical care provider can configure the formatting and display of data intended for the medical records dashboard. This simplified interface editor embodies “What You See Is What You Get” (WYSIWYG) methodology, in some embodiments including drag and drop of Flowsheet elements. Upon completion of a Template for the medical records dashboard, associated parameters can be defined. The Flowsheet Editor enables a user to define how columns and rows will be displayed in the medical records dashboard. While users have the ability to only view predefined data, filtered data may trigger a rule to display in lieu of predefined filters. Preconditioned upon required contract/agreement between users, data can display from multiple, disparate sources to display continuity of care.

In some embodiments, the Flowsheet Editor Interface of the Data Command Center also enables an end-user to configure how summary rows are presented within the medical records dashboard. A user can choose to discard certain edge-cases from the summary calculation, take the highest and lowest values, take an average, or some other logical calculation to determine how the individual columns summary row data presents itself. In addition to enabling changes which reflect the display of the data, it is possible for the user to program alerts and auto-tasks which are sent as a result of the rule threshold being exceeded. For example, if a user/medical care provider determines that a new alert rule must be created, they are able to select the column, or columns, apply logical rules to the column or columns being analyzed, and set the task associated with rule which will be sent to the user-defined staff member or groups of staff members. As another example, a user can choose to add a new alert for those patients which have a diagnosis of Glaucoma and have not had a required diagnostic test, a Visual Field, in 365 days. The user is then able to set a task that will be sent to staff to schedule the diagnostic test that will automatically be sent when the system and user-defined auto task and alerts are processed. The editor interface also enables a user to configure a manner in which the alert is displayed in the medical records dashboard. For example, a user can set the display of an alert to any of the following, but not limited to, headers, within the rows and columns of the flowsheet, on the patient demographic panel, etc.

Pre-defined display rules can override a user-defined configuration of a medical records dashboard when the rule is prioritized, in some embodiments, for patient safety reasons. These overrides can display information regarding a subject visit in a prominent color. For example, if a patient had recently found out that she was pregnant, it becomes very important that she does not have certain diagnostic tests performed as such tests can endanger the viability/health of the fetus. For example, a Fluorescein Angiogram should not be performed on a patient to monitor the progress of a patient if she is pregnant. Due to the potential life altering consequences, the Data Command Center, through the use of, for example AI, is smart enough to override a medical records dashboard template, and prominently display the visit from the OBGYN on the medical records dashboard, in an instance in which the patient was confirmed to be pregnant.

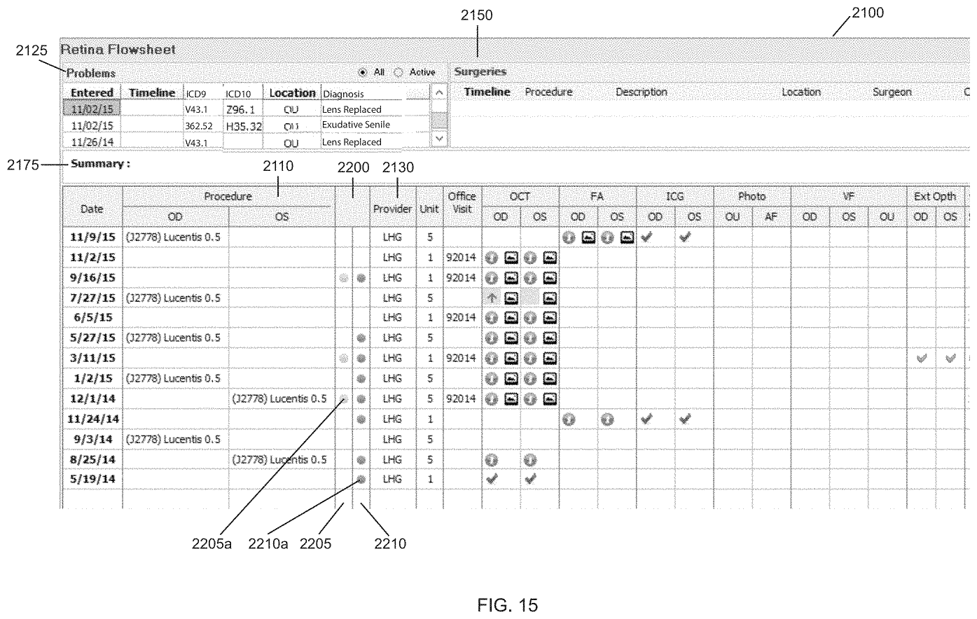

In some embodiments, a Data Command Center of the present principles can enable a user to access a detailed ledger comprising patient financial information from a medical records dashboard. In some embodiments, the medical records dashboard can include at least one visual indication of a payment for services provided, where detailed information of the charges, payments, write-offs, adjustments, and balances can be accessed and displayed. For example, FIG. 15 depicts an embodiment of a medical records dashboard 2100 which can be displayed following a user's selection of at least one medical records dashboard from the medical records dashboard selection window in accordance with another embodiment of the present principles. For example, in some embodiments, the user can make a selection of “Retina Flowsheet” to access and/or launch the medical records dashboard 2100. In some embodiments, the medical records dashboard 2100 can include a display of data from one or more medical records and can track medical procedures and services based on claims made or billing signed off by a physician for one or more delivered medical procedures or services. Some embodiments include a Data Command Center that can dynamically link to various external databases comprising patient information that can be displayed in the medical records dashboard 2100. For example, in some embodiments, the Data Command Center can function as a portal to patient information prepared by the user or patient information from other sources. Further, in some embodiments, the medical records dashboard 2100 can be auto-populated as a function of claims made or billing signed off by a physician. In this instance, any data displayed within the medical records dashboard 2100 is derived from one or more claim records that have been billed for one or more procedures or services have previously been provided to the patient. In some other embodiments, auto-population can be enabled in both directions interacting as a switchboard between the entire EMR and the medical records dashboard 2100.

In some embodiments, the medical records dashboard 2100 can display information related to medical procedures or services in relation to retinal eye care of a patient. In other embodiments, a medical records dashboard can display information related to medical procedures or services in relation to any kind of medical care of a patient. In some embodiments, the medical records dashboard 2100 can display various windows and sub-windows based on a user preference and/or current or previous user interaction with the medical records dashboard 2100. For example, in some embodiments, the medical records dashboard 2100 can display a problems window 2125 and/or a surgeries window 2150 where information related to a patient's medical problems and surgeries can be displayed.

In some embodiments, the medical records dashboard 2100 of FIG. 15 can display information including components where there is a summary of the patient's problem list in which a user can input patient information and constantly update and change. Further, this information can be auto-populated with the touch of a button into a designated location such as the current plan documenting the patient's current visit (thus aiding documentation for the current visit). Further, whatever is important for a user to input into the day's visits for documentation can be initially inputted in the table, and then permanently into the day's patient visits. Further, the summary section of the medical records dashboard 2100 can be constantly fluid and can be changed at every visit rather than being written to an unchangeable document or file (e.g., such as a PDF). For example and as depicted in FIG. 15, the medical records dashboard 2100 can include a summary window enabling a user to view and edit summary information related to the patient, any details of care provided to the patient, and/or and any medical diagnosis information prepared by a medical practitioner. Further, the medical records dashboard 2100 can also display detailed information related to any medical procedures or services provided to the patient, including procedures or services that are auto-populated by claims made, or billings or payments including billing signed off by a physician as detailed above. Additionally, all of the features of the previously described medical records dashboards of the present principles can be provided in the medical records dashboard 2100.

Some additional features of a medical records dashboard of the present principles, such as the medical records dashboard 2100 of FIG. 15, include displaying at least one visual indication of a payment for services provided. Further, the user can be provided with access to a detailed ledger comprising financial information related to one or more procedures. For example and as depicted in FIG. 15, the medical records dashboard 2100 can comprise a payment indicator column 2200 including one or more indicator and/or access icons. For example, in some embodiments, the payment indicator column 2200 can comprise a column 2205a that can be populated with one or more indicator icons. In other embodiments, the column 2205b can be provided with one or more indicator or access icons. In some embodiments, the one or more indicator or access icons can comprise icons of color such as red, yellow, or green to indicate a status of payment from an insurer, varying colors to account for patient payments such as gray for items of no concern, blue for a middling balance or low balance that is past due, or black for a high balance or middling balance that is past due. Other indicator or access icons can display messages such as denial code descriptions or useful messaging, and the whole field may be highlighted in cases where indicator or access icons require additional notification. The payment indicator column 2200 can be located anywhere on the of the medical records dashboard 2100. In the embodiment of FIG. 15, the payment indicator column 2200 is positioned between the procedure column 2110, illustrating or providing access to information detailing one or more procedures performed on the patient and information related to the medical care provider, and the provider column 2130, that can display the location where the procedure was performed, and office visit information. The payment indicator column 2200 may also be flagged or alerted when key parameters are met.

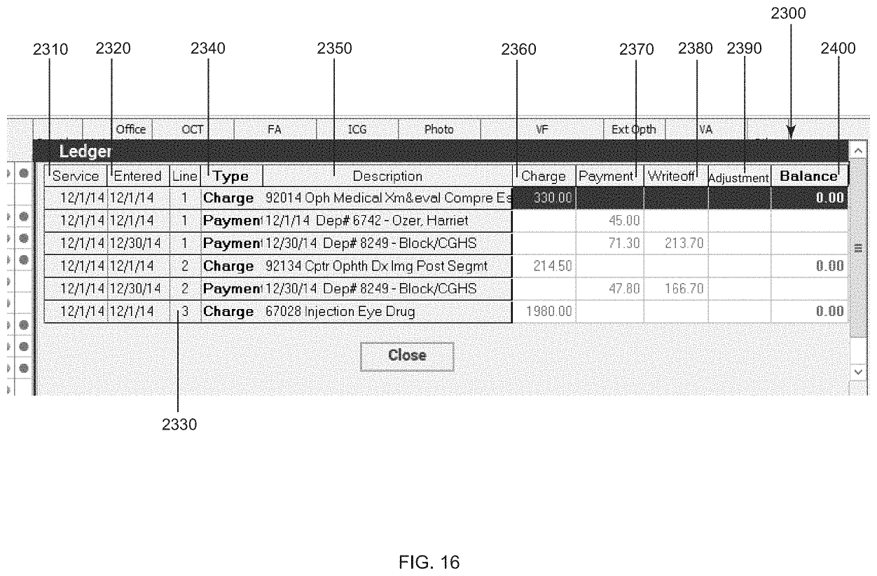

In some embodiments, one or more of the icons of the payment indicator column 2200 can be accessed by the user to initiate the display of more detailed financial information. For example, FIG. 16 depicts a ledger window 2300 accessible from the medical records dashboard 2100 of FIG. 15 in accordance with an embodiment of the present principles. In some embodiments, the Data Command Center of the present principles can display the ledger window 2300 overlaid onto the medical records dashboard 2100. In other embodiments, the ledger window 2300 can be displayed in place of the medical records dashboard 2100. In other embodiments, the ledger window 2300 can be displayed with the medical records dashboard 2100. In some embodiments, the ledger window 2300 can include information processed by the Data Command Center, which includes information related to the date of procedure, description of the procedure, dates entered, date of last transaction, a charge type, modifiers, charge status, etc. and important line items may be highlighted through color changes, alerts, flags, or other digital or textual notifications to draw attention to important details and/or trends. For example, and as depicted in the embodiment of FIG. 16, the ledger window 2300 can include the service to column 2310, entered column 2320, line column 2330, type column 2340, and description column 2350. Further, in some embodiments, the ledger window 2300 can include information related to payments and billing. For example, in some embodiments, the ledger window 2300 can include a display of a charge column 2360, payment column 2370, write-off column 2380, adjustment column 2390, and a balance column 2400, although any relevant financial data may be included. In some embodiments, the user can close the ledger window 2300 and return to the medical records dashboard 2100 at any time. In other embodiments, more than one ledger window 2300 can be displayed based on selections made by the user in the medical records dashboard 2100.

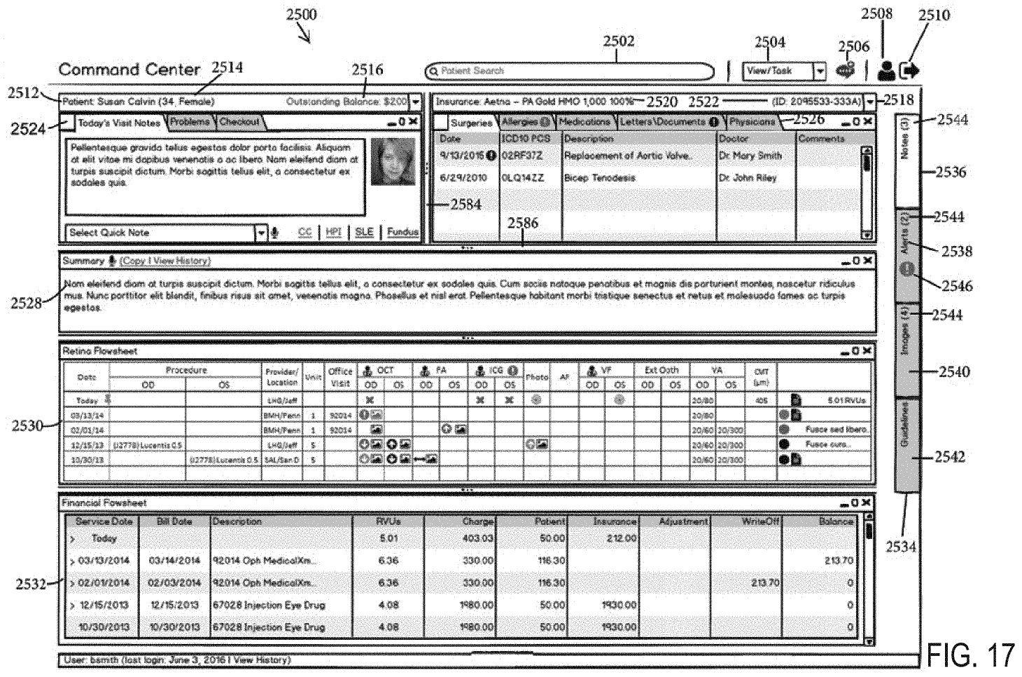

FIG. 17 depicts an embodiment of a Data Command Center menu 2500 including a medical records dashboard 2530 implemented as a data interface to a medical record system in accordance with an embodiment of the present principles. The Data Command Center menu 2500 in the embodiment of FIG. 17 is designed to interact with a conventional EMR system although, as noted above, the Data Command Center menu 2500 of FIG. 17 can be used with other large data systems, such as a Practice Management System, to present data to users in a meaningful way. In addition, the exemplary embodiment illustrates a Data Command Center menu 2500 for implementation in an Ophthalmology practice. Those skilled in the art will appreciate that the interface can be readily configured for other medical specialties. The Data Command Center menu 2500 is able to display data from multiple data sources in multiple different panels on a single interface. In the exemplary embodiment of FIG. 17, the Data Command Center menu 2500 provides a comprehensive overview of the patient's clinical and financial history as well as providing a means to quickly order tests while retaining the ability to see previous medical and financial history. Clinical, insurance, and regulatory guidelines, as well as preferred practice and billing patterns, can be quickly accessible based on the patient's conditions, medications and procedures so that a medical care provider/user can readily provide optimal care and be compliant with medical and billing requirements. The medical care provider thus becomes a part of revenue cycle management for each patient in the medical care provider's practice. Through preprogrammed alerts, notifications, and tasks, or alerts, notifications, and tasks programmed at point of care, the medical care provider has a simplified means of being instructed when key aspects of revenue cycle management diverge from expected results.

A Data Command Center and medical records dashboard in accordance with the present principles can incorporate self-deleting staff messages that are presented to the Data Command Center. For example, a staff person can send a message about a patient to the medical care provider that appears in a display window in either of the Data Command Center menu 2500 and the medical records dashboard 2530 with a message such as “the patient has been waiting over an hour and is upset” or the “patient has previously filed a malpractice complaint” that do not become part of the patient's medical record. The message can be programmed to be deleted once the patient's visit is billed.

As depicted in the embodiment of FIG. 17, a user has the ability to search for patients at 2502, select different views of the data at 2504, add sticky notes at 2506, access user information at 2508, and logout at 2510. Immediately below that on the upper left-hand side of the Data Command Center menu 2500 is the Patient Information Bar 2512, which contains the patient's identifying information 2514 so the user knows they are looking at the correct patient record. The Patient Information Bar 2512 also notifies the user of patient's outstanding balance 2516. To the right of the Patient Information Bar 2512 is the Patient Insurance Bar 2518, which provides the patient's insurance information 2520, including the ID number 2522 for the patient's primary insurance. Below the Patient Information Bar 2512 is a collection of tabs 2524 displaying different sets of information about the selected patient. A different collection of tabs 2526 is found underneath the Patient Insurance Bar 2518. Under tabs 2524 is the Summary panel 2528 where the user can enter notes about the patient (e.g. patient did not show up for missed appointments).

The medical records dashboard 2530 (illustratively depicted as a Retina Flowsheet), is an encounter driven panel that summarizes key clinical and financial information in chronological or reverse chronological order at a glance, allows the user to order new Procedures and Imaging tests, and provides assistance complying with insurance regulations, as will be described in more detail below. Below the medical records dashboard 2530 is the Financial Flowsheet 2532 providing a summary of the financial information related to the patient and this is adapted to provide the user with the ability to drill down into individual transactions. On the right side of the medical Data Command Center menu 2500 are a series of vertical tabs 2534 that when individually clicked slide out to provide more information to the user. The Notes Tab 2536 expands to display patient notes, while the Alerts Tab 2538 expands to display patient alerts (e.g. patient's chart was requested by insurance company or sent for a second opinion). The Images Tab 2540 expands to display images for the patient and the Guidelines Tab 2542 expands to display clinical practice and insurance guidelines along with preferred practice patterns where applicable. On each tab, displayed next to the tab title is the count of the new items 2544 since the last time the user accessed the patient record. Once the tab is expanded, the new count will be removed because the user has seen the information. If important or critical data exists within the tab, a special alert icon 2546 is displayed on the tab (describe modules and details of this function). Once viewed, the alert icon 2546 is removed. As will become apparent from the following description, the layout of the Data Command Center menu 2500 permits access by the user to all of the relevant information within one click of the mouse and without having to steer to other screens that would take the user away from the Data Command Center menu 2500. For example, the information is either available in a display window, behind a tab, or available via a pop-up window; accordingly, the user does not have to leave the display screen to access the information (describe modules and details of this function).

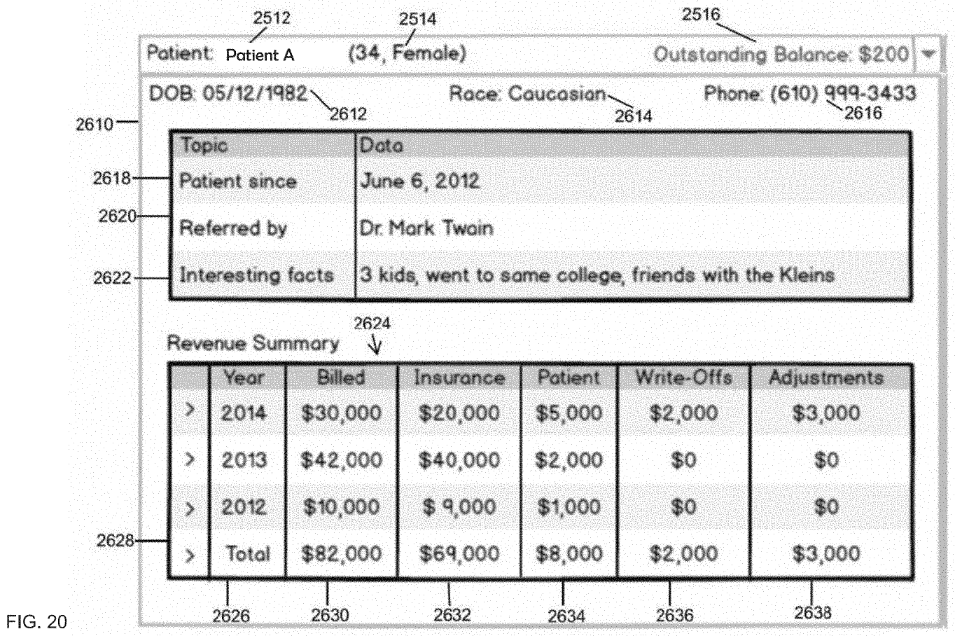

The Patient Information Bar 2512 illustrated in FIG. 17 displays high level information about the patient. The user can click on the Patient Information Bar 2512 and the Patient Information Panel 2610 shown in FIG. 20 is displayed underneath the Patient Information Bar 2512. Clicking the bar a second time or anywhere else on the page will close the Patient Information Panel 2610. The Patient Information Panel 2610 contains the patient's date of birth 2612, race 2614, phone number 2616, the date when the patient was first seen 2618, the referring physician 2620, and interesting facts to remember 2622. Information in the Patient Information Panel 2610 can be edited in place by clicking on the item to be changed. When clicked, the field will change to a text field allowing the value to be changed with a Save button displayed next to it (not depicted) or allow for auto-save functionality upon leaving the field. Clicking Save will save the data and/or auto-saving may be enabled to allow saving the data upon exiting the field. Clicking a Cancel button (not shown) will not save the data leaving the value unchanged and the control will revert to static text when auto-saving is not enabled on the field. Below these fields is a Revenue Summary 2624 for the patient. The Revenue Summary 2624 displays patient totals for each year 2626 as well as grand totals 2628 for the total amount billed 2630, amount paid by insurance 2632, amount paid by the patient 2634, amount written off 2636, and the adjustments 2638 for each year. Any or all of these values may be alerted or tasks may be generated based on preconfigured rules or rules configured within the context of the panel.

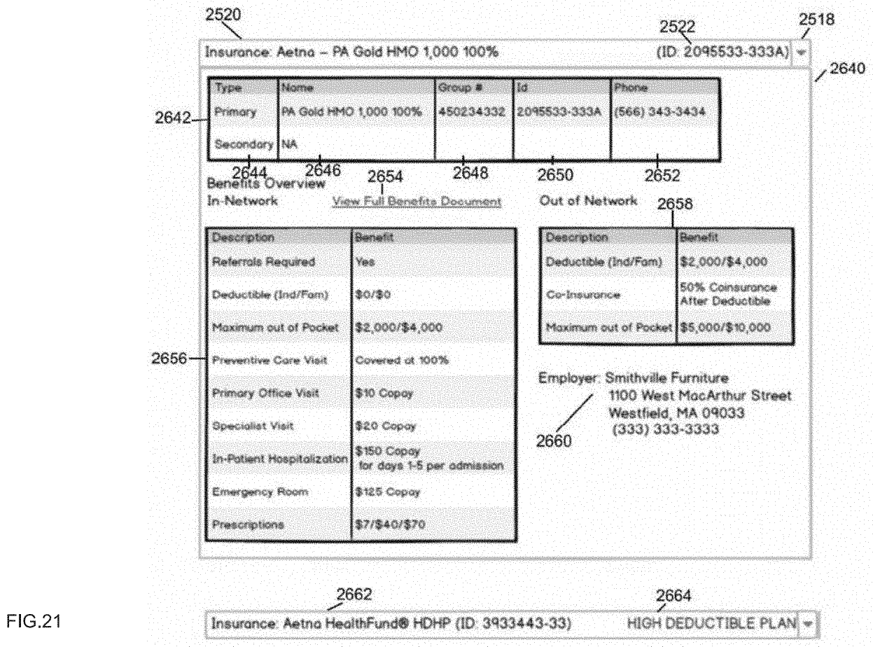

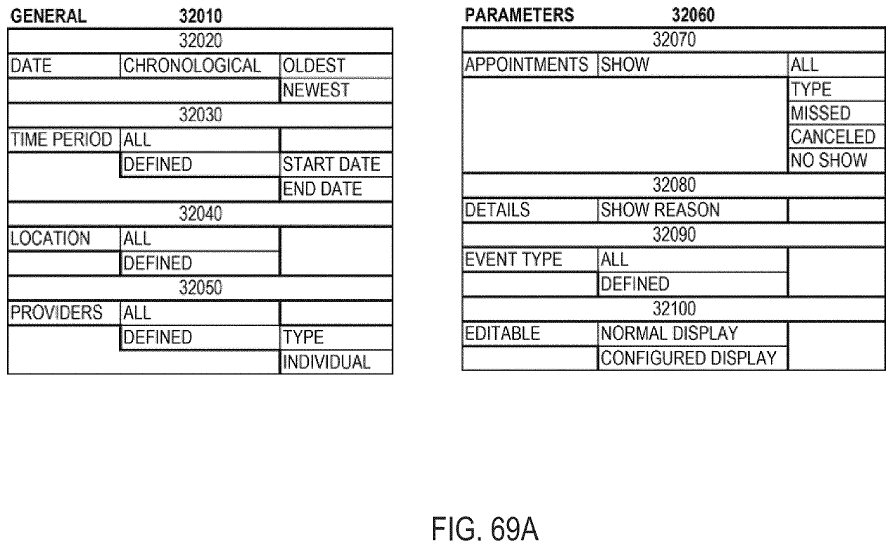

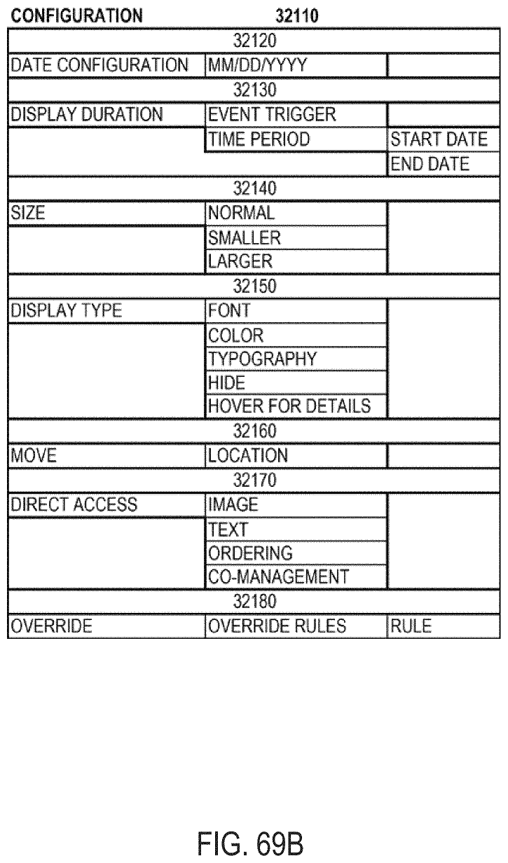

To the right of the Patient Information Bar 2512 in FIG. 17 is the Patient Insurance Bar 2518. The user can click on the bar and the Patient Insurance Panel 2640 illustrated FIG. 21 is displayed underneath the Patient Insurance Bar 2518. Clicking the Patient Insurance Bar 2518 a second time or anywhere else on the page will close the Patient Insurance Panel 2640. The Patient Insurance Panel 2640 contains information about the patient's insurance 2642 including the type 2644, name 2646, group number 2648, insurance ID number 2650, and phone number 2652 for each insurance company. A link to the patient's benefit document 2654 is provided as well as an overview of the patient's in-network benefits 2656 and out of network benefits 2658 as provided by the patient's insurance company. The values illustrated as 2656 and 2658 are provided for example purposes and will vary based on the data provided by insurance companies. The patient's employer's address and contact information 2660 is also displayed for convenience. Item 2662 displays an alternative embodiment of the Patient Insurance Bar 2520. In this case, the patient has a high deductible plan and this fact is displayed at 2664 in red in the Patient Insurance Bar 2520 to be sure the physician is aware that, in this case, the patient has a high deductible plan. The intelligent alert configuration system illustrated in FIGS. 69A and 69B depict may be launched to configure rules, alerts, notifications, and tasks based on clinical and/or financial parameters.

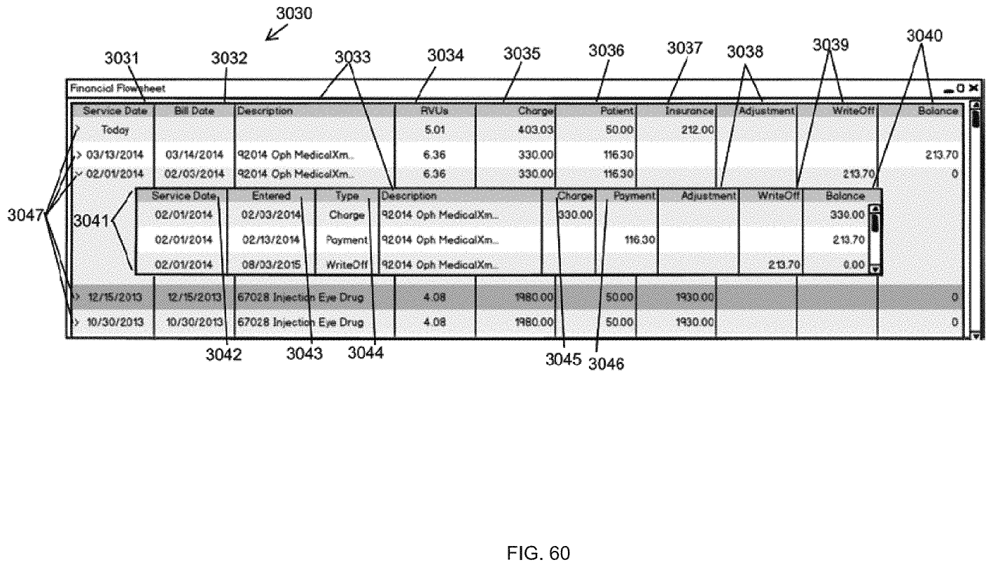

The Financial Flowsheet 2532 is illustrated in FIG. 60. The Financial Flowsheet 2532 contains a dynamic table 3030 of financial activity for the patient, as opposed to the physician's practice, as is usually the case with conventional EMR systems. As illustrated, an exemplary embodiment of the Financial Flowsheet 2532 includes the following fields: the Service Date 3031, Bill Date 3032, Service Description 3033, Relative Value Units (RVUs) 3034 that determine how a physician is paid in accordance with work effort, Gross Charges 3035, the Amount Due from the Patient 3036, the Amount Due from Insurance 3037, any Adjustments 3038, Write Offs 3039, and the outstanding balance 3040. An intelligent alert configuration system can be launched to configure rules, alerts, notifications, and tasks based on clinical and/or financial parameters denoted in FIG. 60. The rules, alerts, notifications, and tasks may be applied to one or a group of patients. Selection of the Encounter expansion control 3047 produces a second dynamic table 3041 within the first dynamic table 3030. The second table 3041 presents individual financial transactions associated with the encounter. In an exemplary embodiment, the second dynamic table 3041 contains: the Service Date 3042, Date Entered 3043, the Type of financial activity entered 3044, the Service Description 3033, the Charge or Amount Billed 3045, Payments 3046, Adjustments 3038, Write Offs 3039, and the outstanding balance 3040. The intelligent alert configuration system illustrated may be launched to configure rules, alerts, notifications, and tasks based on clinical and/or financial parameters at a more granular level, which may then be applied to one or a group of patients.

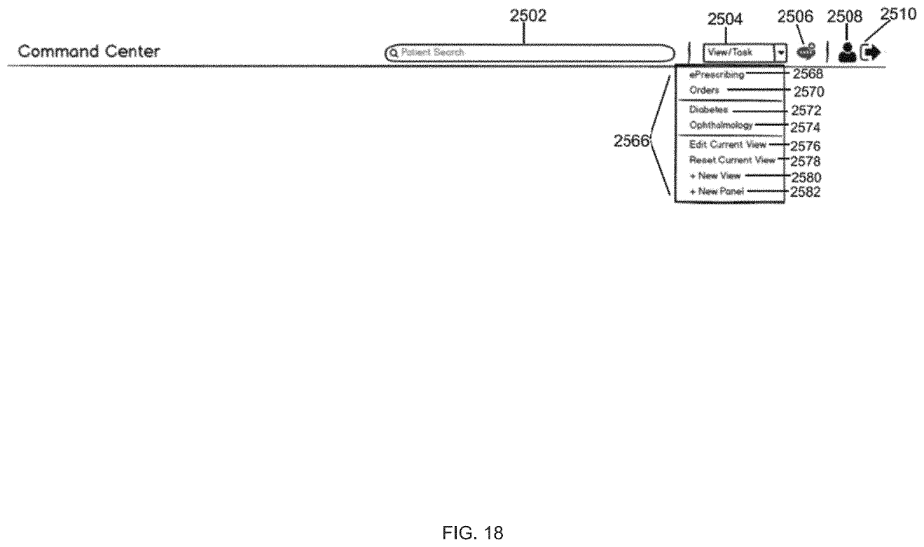

FIG. 18 depicts a User View control panel 2566 that can be part of the View/Task menu 2504 of the medical records dashboard of the Data Command menu of the embodiment of FIG. 17 in accordance with an embodiment of the present principles. The user View control panel 2566 of FIG. 18 displays views for selection by the user. As illustrated, the user can select one of several Views 2568-2574. Views ePrescribing 2568 and Orders 2570 are examples of task-based views, while Diabetes 2572 and Ophthalmology 2574 are examples of condition or specialty specific views. In an exemplary embodiment, the system has several pre-configured views but more can be added over time by deploying new versions of the system or by user modification. The user can edit the Current View 2576, Reset the Current View 2578 to its default configuration, create a new View 2580, or create a new Panel 2582. If the user selects a new view from options 2568-2574, the screen layout is changed to reflect the selected view for the current patient. (describe modules and details of this function). Referring back to FIG. 17, it should be noted the dimensions of the different panels can be resized by changing the location of the vertical sliders 2584 and horizontal sliders 2586. This enables the user to control how space/area on the display is used for each panel. As the user adjusts the sliders 2584 and 2586, the dimensions are remembered so when the user returns to the view at a later time the system remembers the dimensions. The user also can reset the view to its default dimensions by clicking the Reset Current View 2578 option in the view control panel 2566. The entire view also resizes based on the dimensions of the user's monitor and the size of the browser display.

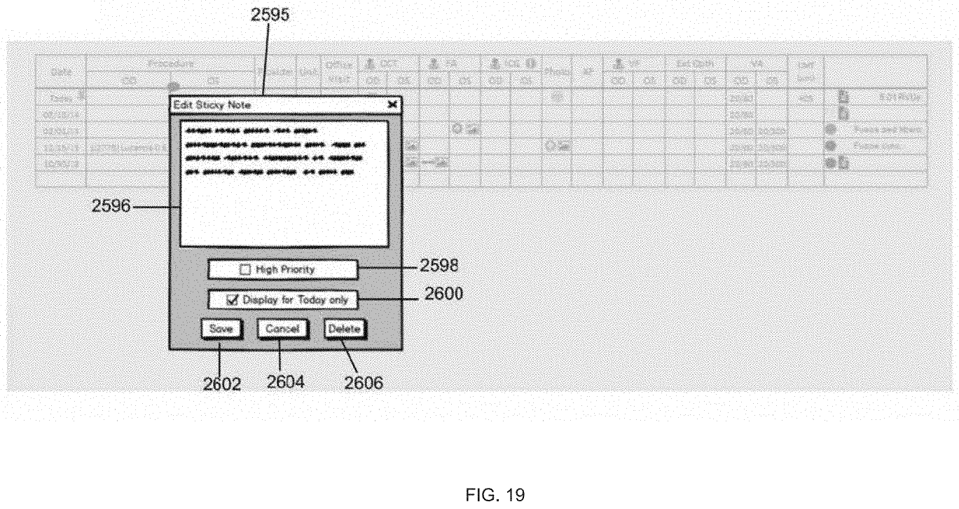

FIG. 19 depicts a sticky note panel 2595 of the Data Command Center menu 2500 of FIG. 17, which is activated when the add sticky notes icon 2506 in FIG. 17 is selected in accordance with an embodiment of the present principles. As illustrated, the user may click and drag the icon 2506 to any location on the page and drop it where they want it placed. This allows the user to place the note in context of the information to which it refers. The icon 2506 then stays in place and is associated with dynamic data until deleted by a user or it expires based on the note settings. (describe modules and details of this function). When the icon 2506 is placed, the Edit Sticky Note control panel 2595 is displayed. The Edit Sticky Note control panel 2595 can also be displayed if a user selects the icon 2506. Once the Edit Sticky Note control panel 2595 is opened, the user can enter a note in field 2596, select if the note is high priority 2598, select if the note should only be displayed today 2600, in which case after the date it is entered the note will no longer display. The user can save the note by clicking the Save Button 2602 or cancel the action by clicking the Cancel Button 2604. The user can delete the note by clicking the Delete button 2606 at which point the action is confirmed before deleting. When a user moves the mouse over the note icon 2506, the note text is displayed next to it in a tooltip. The note icon is colored, e.g., black if it is not high priority and, e.g., red if it is high priority or can flash or be highlighted in any other manner. All deleted or expired Sticky Notes along with the location and duration where they are displayed can be preserved for purposes of legal discovery but may not be accessible to the user as a general practice.

Referring back to FIG. 17, when the user selects the user profile icon 2508, a panel (not shown) is presented to enable the user to edit typical user information including their name, address, phone numbers, email address and password. The user can also select their preferred email or phone number or other method for communications sent by the Data Command Center of the present principles. When the user selects the Logout icon 2510, if any data is not saved, the user can be prompted to save data before closing the system. If the user answers positively that they want to save unsaved data, the user is not logged out. Once all data is saved, the user is prompted to confirm their desire to logout. If confirmed, the user is logged out.

The Patient Information Bar 2512 illustrated in FIG. 17 displays high level information about the patient. The user can click on the Patient Information Bar 2512 and the Patient Information Panel 2610 shown in FIG. 20 is displayed underneath the Patient Information Bar 2512. Clicking the bar a second time or anywhere else on the page will close the Patient Information Panel 2610. The Patient Information Panel 2610 contains the patient's date of birth 2612, race 2614, phone number 2616, the date when the patient was first seen 2618, the referring physician 2620, and interesting facts to remember 2622. Information in the Patient Information Panel 2610 can be edited in place by clicking on the item to be changed. When clicked, the field will change to a text field allowing the value to be changed with a Save button displayed next to it (not depicted). Clicking Save will save the data. Clicking a Cancel button (not shown) will not save the data leaving the value unchanged and the control will revert to static text. Below these fields is a Revenue Summary 2624 for the patient. The Revenue Summary 2624 displays patient totals for each year 2626 as well as grand totals 2628 for the total amount billed 2630, amount paid by insurance 2632, amount paid by the patient 2634, amount written off 2636, and the adjustments 2638 for each year.

To the right of the Patient Information Bar 2512 in FIG. 17 is the Patient Insurance Bar 2518. The user can click on the bar and the Patient Insurance Panel 2640 illustrated FIG. 21 is displayed underneath the Patient Insurance Bar 2518. Clicking the Patient Insurance Bar 2518 a second time or anywhere else on the page will close the Patient Insurance Panel 2640. The Patient Insurance Panel 2640 contains information about the patient's insurance 2642 including the type 2644, name 2646, group number 2648, insurance ID number 2650, and phone number 2652 for each insurance company. A link to the patient's benefit document 2654 is provided as well as an overview of the patient's in-network benefits 2656 and out of network benefits 2658 as provided by the patient's insurance company. The values illustrated as 2656 and 2658 are provided for example purposes and will vary based on the data provided by insurance companies. The patient's employer's address and contact information 2660 is also displayed for convenience. Item 2662 displays an alternative embodiment of the Patient Insurance Bar 2520. In this case, the patient has a high deductible plan and this fact is displayed at 2664 in red in the Patient Insurance Bar 2520 to be sure the physician is aware that, in this case, the patient has a high deductible plan.

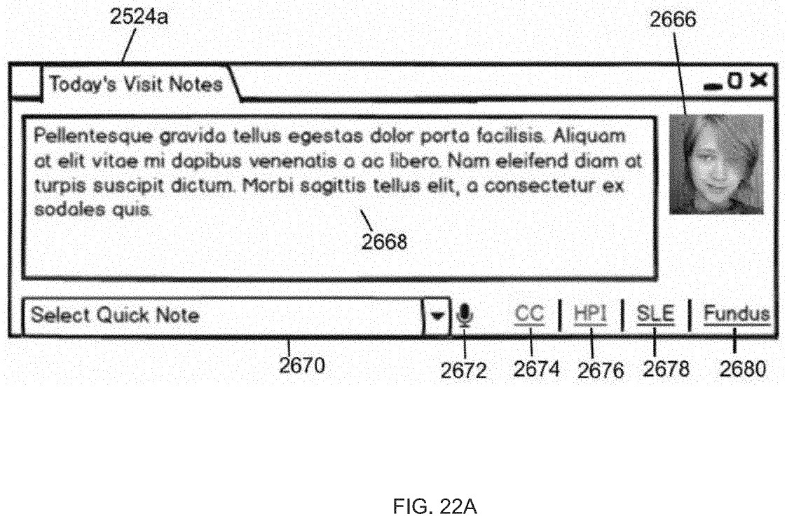

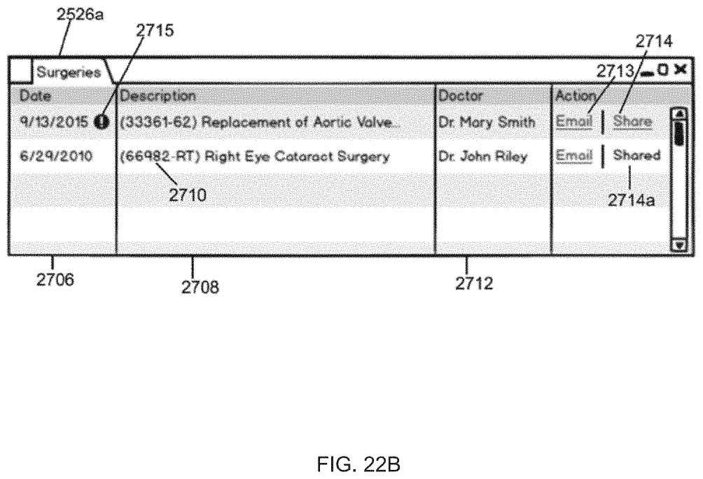

FIG. 22A depicts an embodiment of a Today's Visit Notes tab 2524a of the Data Command Center menu 2500 in accordance with an embodiment of the present principles. The Today's Visit Notes tab 2524a as illustrated in FIG. 22A contains elements related to capturing information about notes specific to today's visit. The tab 2524 contains a photo 2666 of the patient, free form text notes 2668, a control allowing the user to select pre-configured notes 2670, an icon 2672 that triggers a dictation feature allowing text entry into free form text notes 2668 via voice recognition, and a set of links 2674-2680 that are reminders to complete important aspects of an encounter in the EMR. The patient image 2666 is imported from the EMR and text notes 2668 can also be imported from the EMR through the Command Center CCOW Implementation described below. Items 2674-2680 display the status of the chief compliant (CC) 2674, history of present illness (HPI) 2676, slit lamp exam (SLE) 2678, and Fundus photograph 2680. This status is also provided to the Command Center via the CCOW implementation. Items 2674-2680 are displayed in red until complete at which time they are displayed in black. Based on EMR access and functionality, items 2674-2680 are links back to the specific area in the EMR. In an exemplary embodiment, the physician may dictate or type notes into the Today's Visit Notes tab 2524a that automatically generates a letter to a referring physician or another physician alerting that physician about something important in the patient's medical history. Beneficially, the referring letter may be generated while the patient's medical history is on the display screen in the Data Command Center interface.

Embodiments of the present principles can further include a Problems tab, which displays a patient's problem list as imported from the EMR. The following fields can be displayed, including but not limited to; date entered, associated ICD10 code, body location, and diagnosis. The user can manually order the list in order of severity or importance by clicking and dragging the rows. A Sort by Date can sort the list in reverse chronological order and Sort by Importance c a n sort the list using the user's manual ordering. If the user has not adjusted the order of Problems, it will display in reverse chronological order. A default sort order can be by date, but, in some embodiments, the user's last selection is remembered and automatically selected when the user returns to the application.

Embodiments of the present principles can further include a Checkout tab used to determine when a patient should return to the practice. This can also be used for a return visit to a shared physician's office which would then also in some embodiments populate a shared care medical table that can be given to a patient for a future reminder of appointment. The Checkout tab can be configured to display a recommended clinical guideline based on Clinical Decision Support algorithms of the Data Command Center. A user can select a count and a period to generate a time period in which the patient should return. A search feature can implement basic type-ahead search and results listing enabling the user to select an item. In the case of either the search or a drop-down menu the selected item can be listed underneath. The user has the ability to delete the item by clicking an associated delete icon. The user can also enter a free form text note or use dictation by selecting a dictation icon 2702. When complete, the user can click a Save button to save the Checkout information and send it to the EMR or clear the information by clicking a Clear button.

FIG. 22B depicts an embodiment of a Surgeries tab of a medical records dashboard of a Data Command Center in accordance with an embodiment of the present principles. The Surgeries tab 2526a as illustrated in FIG. 22B displays information about the patient's surgeries. The Surgeries tab 2526a displays the date of surgery 2706, the description 2708 including the billing (ICD10) code 2710, the primary physician 2712, and several actions including the ability to email 2713 or share 2714 the patient record with another physician. The shared notation 2714a signifies that the patient record has already been shared with the other physician. A notes column (not shown) displays the first few characters or words based on available space of an associated note. Moving the mouse over the specific note displays it in a pop up. In the case of some surgeries, a hospital or physician will not be paid if readmitted in 30 days. In these cases, if a surgery has been performed in the last 30 days a black circle with an exclamation point 2715 can be displayed next to the date. Moving the mouse over the icon displays a message stating how many days are left until the patient can be readmitted. An associated note can also indicate that the patient is participating in a capitated plan where anything the physician orders for the patient will not be reimbursed.

When a patient record is shared with another medical professional, if the professional does not have access to the D a t a Command Center of the present principles, the other medical professional can receive an email to register for access to the Data Command Center. In some embodiments, if the professional does have an account but a new patient is being shared, the physician can receive an email notification. The new external user will only have access to the specific patients that are shared. Such sharing of patient medical records amongst the patient's physicians better enables the physicians to work together to follow preferred practice patterns for patient treatment as may be required by insurance companies and/or the government. This process is particularly helpful for managing patients with certain chronic diseases like diabetes in which a nephrologist, podiatrist, ophthalmologist, endocrinologist, and family physician need to see each other's results. Another example is shared care before and after cataract surgery where optometrists and ophthalmologist need to see each other's results.

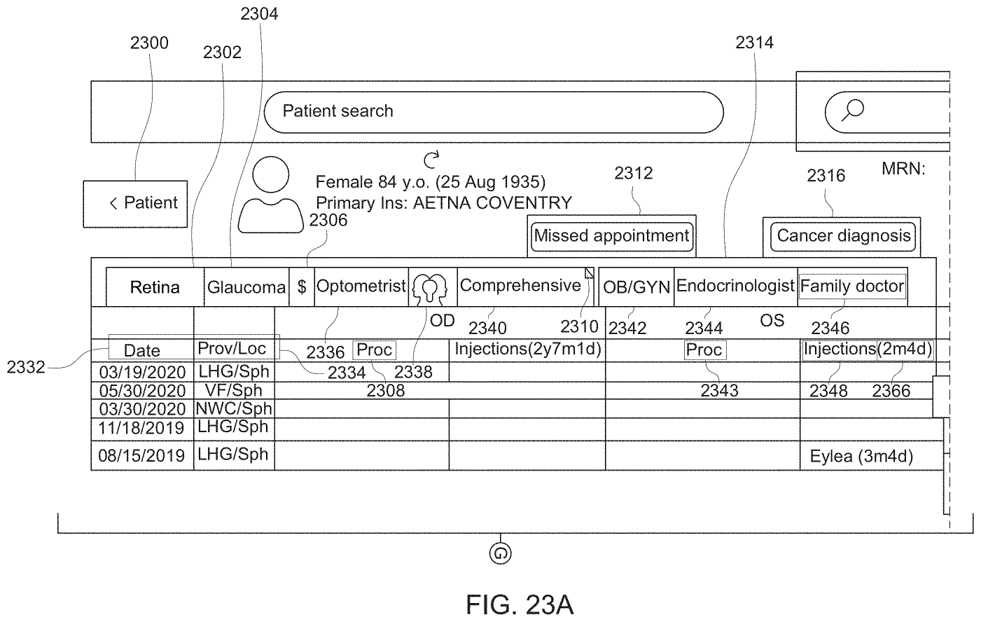

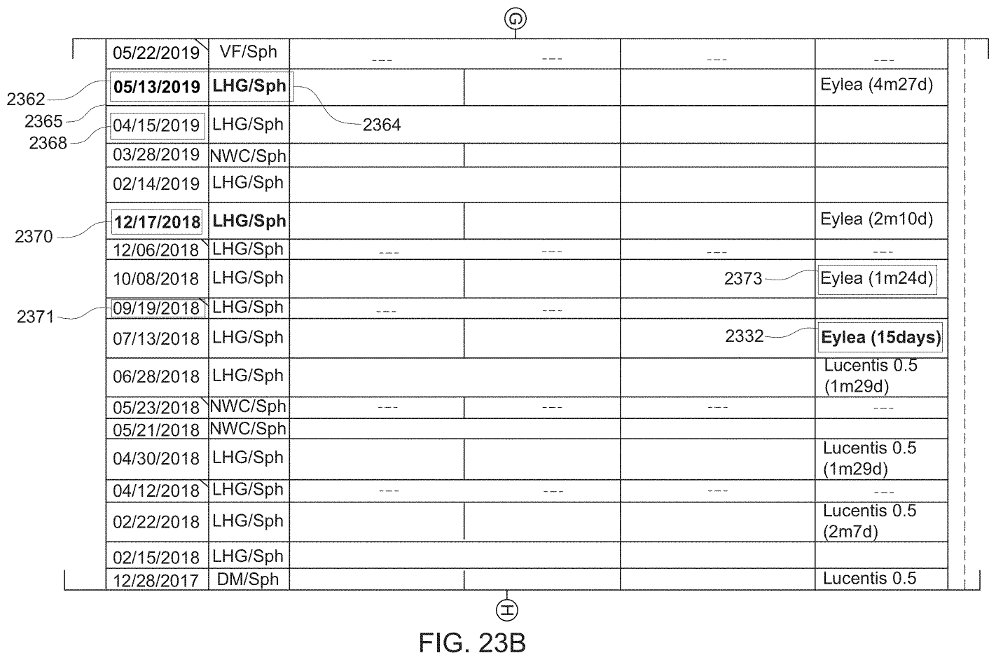

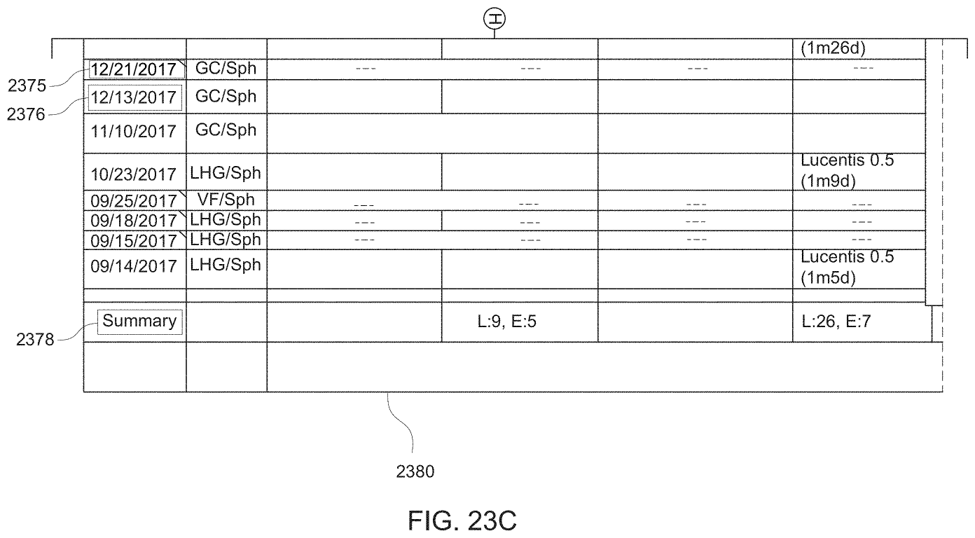

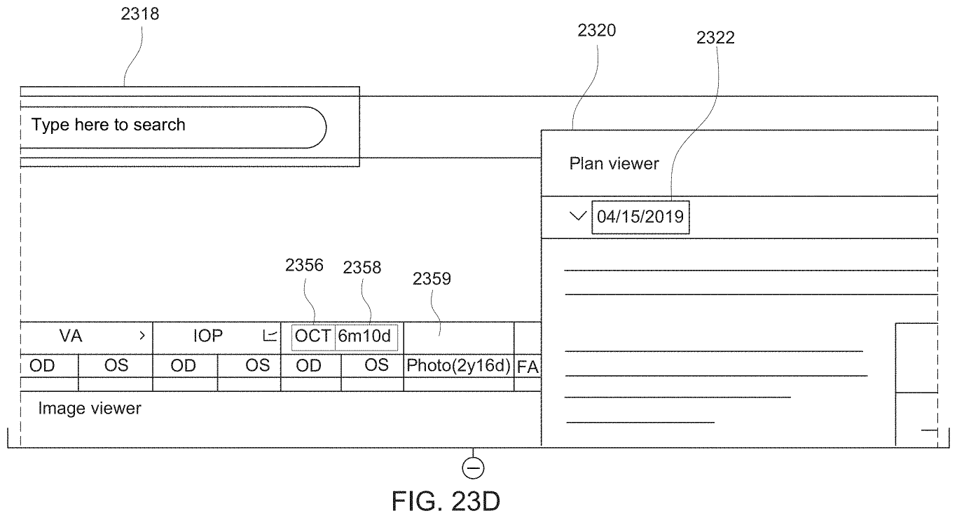

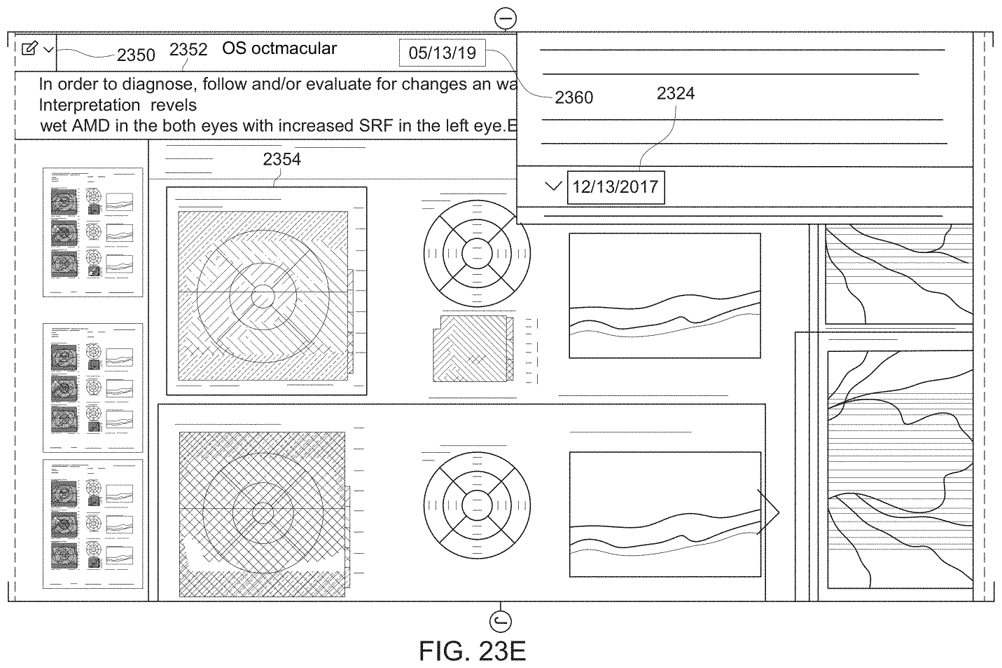

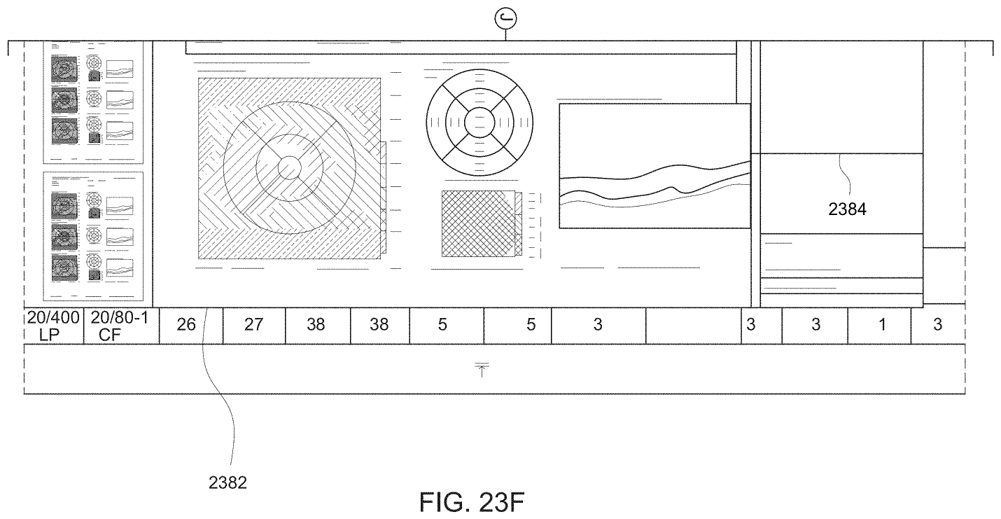

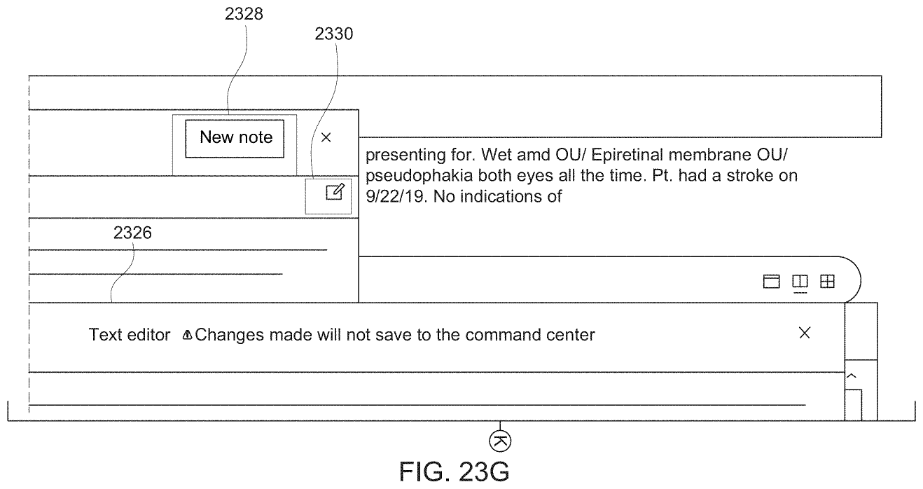

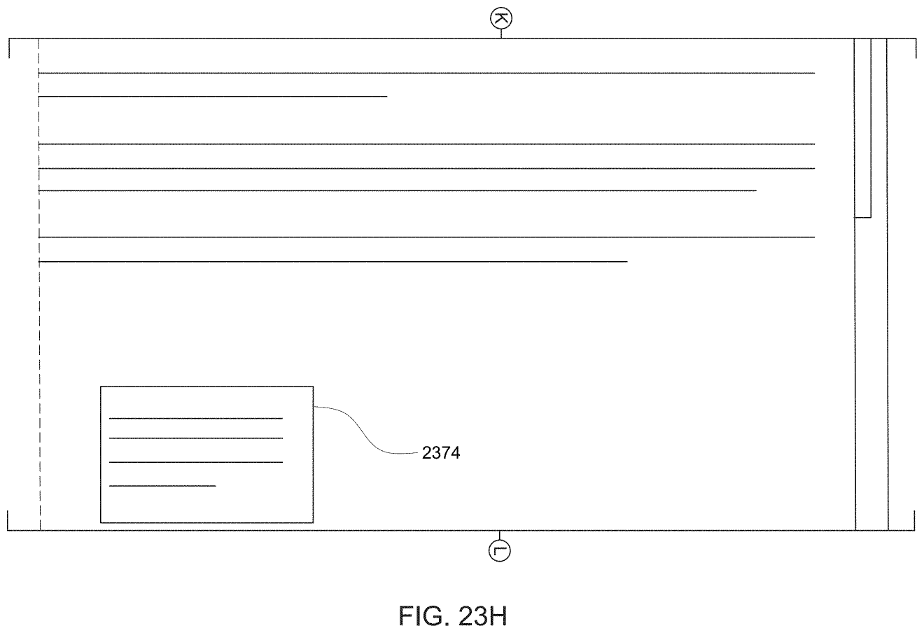

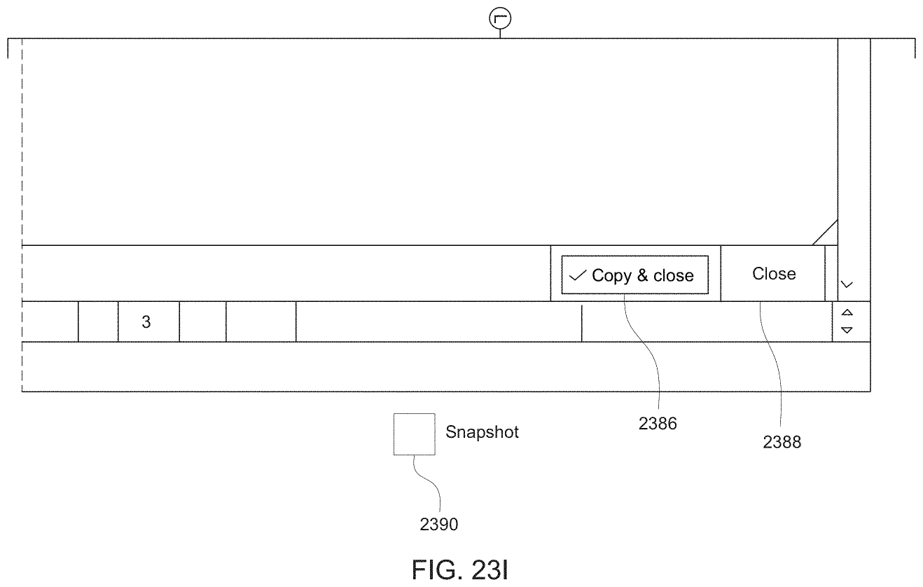

FIGS. 23A, 23B, 23C, 23D, 23E, 23F, 23G, 23H, and 23I depict an embodiment of a medical records dashboard in accordance with another embodiment of the present principles. In accordance with the present principles, the medical records dashboard of FIGS. 23A, 23B, 23C, 23D, 23E, 23F, 23G, 23H, and 23I depict is intended to provide and display to a user/medical care provider with all patient data/information necessary to perform accurate and efficient patient care using a single display. In the embodiment of the medical records dashboard of FIGS. 23A, 23B, 23C, 23D, 23E, 23F, 23G, 23H, and 23I, panels 2380, 2382, 2326, 2320, and 2314 are some examples of different panels that can be moved around, toggled, simultaneously active (i.e., information from each panel can be assessed interchangeably without changing views) and displayed while critical information is viewed. In each column, what is an important data element over time can be followed as noted in column 2332. This enables a user to view the information vital to evaluation of their patients. In addition, in some embodiments, the medical records dashboard of FIGS. 23A, 23B, 23C, 23D, 23E, 23F, 23G, 23H, and 23I enable, direct access to patient data/information (no more than one click, one hover or selected directly in any manner). Some embodiments enable toggling by a mechanism such as alt-tab to gain access to underlying patient data/information or associated screen, tab or window. A user/medical care provider is able to decide what is important to pull up, directly to view, and can move the separate windows or other pop-ups out of the way to view important patient data/information underneath. In one embodiment, a Rules module, such as the Rules module 004 of the Data Command center 001 of FIG. 1, can be configured to know what information for the patient is important, what information must not be blocked, and when information is directly clicked and displayed, enables the movement of a needed columns into a set area on the screen where critical information remains in view. In the embodiment of FIG. 23, an example of two data sets that remain in view is depicted by column 2332, which includes the date of service when an encounter occurred with a patient, and column 2334, which displays the provider and location of encounter. In the embodiment of the medical records dashboard of FIGS. 23A, 23B, 23C, 23D, 23E, 23F, 23G, 23H, and 23I, all of the other columns, such as column 2348, which depicts injections performed on a patient and/or procedures column 2308 can be moved or at least partially covered from display.

Alternatively or in addition, in some embodiments none of the patient data/information is completely blocked from view through the use of transparency viewing. In FIGS. 23A, 23B, 23C, 23D, 23E, 23F, 23G, 23H, and 23I, block 2354 displays an image of an OCT that displays to a user/medical care provider if injections of the left eye are working. In the embodiment of the medical records dashboard of FIGS. 23A, 23B, 23C, 23D, 23E, 23F, 23G, 23H, and 23I, column 2348 is viewed, not blocked, so the user can correlate when the injection (or any procedure of clinical information or diagnostic test) was performed and how it relates to the information that was pulled up, with direct access to any additional information. In some embodiments of a medical records dashboard of the present principles, columns/windows/pop-ups of interest to a user can be moved to another portion of the medical records dashboard where no patient data/information or patient data/information of little or no interest to a user, exists. For example, if the user would also like to compare OCT data 2356 and in particular the left eye, as this example shows injections of certain medications (i.e. Eylea, Lucentis) and column 2348 over time, the user could simply drag 2356 or just 2358 (left eye) over to column 2308, because no data is present in that area of the medical records dashboard. Now all in one view and in a particular section of the medical records dashboard, exists all information that user would need to compare OCTs 2356 over time with injections 2348. In another example, when a photo 2359 is being compared to when an injection is done in the left eye 2348, then 2359, can be moved, dragged or automatically be placed in location for example next to or in place of 2343. A user remains in control and able to move items out of view and by activating icon 2390 can take a snapshot (record) of a current arrangement of the medical records dashboard such that a record of the arrangement can be stored.

Simultaneously, a medical records dashboard of the present principles enables a user/medical care provider to recall and view plans of the past by activating a plan or A&P column or a particular plan in a column. The medical records dashboard of FIGS. 23A, 23B, 23C, 23D, 23E, 23F, 23G, 23H, and 23I enables current and past plans to be simultaneously displayed. As such, in context, a new note could be created in block 2328. A medical records dashboard of the present principles, such as the medical records dashboard of FIGS. 23A, 23B, 23C, 23D, 23E, 23F, 23G, 23H, and 23I, enables images, procedures, dates of service, plan, or any other patient-related data/information, such as clinical measurement, i.e. VA (vision OD 3005—right or OS 3006—left), to be compared in context. By way of example, how a treatment is working as measured by an image, clinical parameter, or any other related data set can be interpreted and noted in the medical records dashboard in at least block 2352, which can be a new interpretation and can be edited by activating icon 2350. In one embodiment a plan viewer can be accessed by activating block 2328 and a new note or the editing of an old exiting note 2330 can be accomplished via a text editor window 2326. In the embodiment of the medical records dashboard of FIGS. 23A, 23B, 23C, 23D, 23E, 23F, 23G, 23H, and 23I, a user/medical record provider is enabled to type or dictate a note 2374 accurately while relevant information is viewed in for example a window. Although in the embodiment of FIGS. 23A, 23B, 23C, 23D, 23E, 23F, 23G, 23H, and 23I the medical records dashboard only provides a user/medical care provider one means for editing notes, in some embodiments, a medical records dashboard of the present principles can provide a user/medical care provider many ways to edit notes.

In the medical records dashboard of FIGS. 23A, 23B, 23C, 23D, 23E, 23F, 23G, 23H, and 23I, panel 2314 enables a user/medical care provider to select to view patient-related data/information from a number of different health care providers, such that patient-related data/information from every medical care provider that has ever cared for a patient can be viewed by, for example, all other specialties who provide care for that patient. For example in FIGS. 23A, 23B, 23C, 23D, 23E, 23F, 23G, 23H, and 23I, a user/medical care provider can select to see patient care data/information related to a retina specialist 2302 and/or a glaucoma specialist 2304. In some embodiments, sharing of patient-related data/information from other users/medical care providers can require permission from at least one of the patient and the other user/medical care provider.

In the medical records dashboard of FIGS. 23A, 23B, 23C, 23D, 23E, 23F, 23G, 23H, and 23I, the panel, arranges patient data/information displayed in rows and columns. Users/medical care providers can have dashboards that are similar in display because the users/medical care providers charge, order, or perform similar CPT codes and often treat similar ICD diagnostic codes. Type of eye doctors are listed in order in this example #2302 (retina), 2304 (glaucoma), 2336 (optometrist), and 2340 comprehensive eye doctor.

In the embodiment of the medical records dashboard of FIGS. 23A, 23B, 23C, 23D, 23E, 23F, 23G, 23H, and 23I, the different users/medical care providers can let all the other providers know something is important by highlighting the tab 2302, 2304, 2342, 2344, and 2346 in the medical records dashboard view of other users/medical care providers. In such embodiments, a user/medical care provider is able to hover or otherwise active the highlighted tab to bring into view a message 2312 that can detail an important aspect of patient care for the corresponding other user/medical care provider. As depicted in FIGS. 23A, 23B, 23C, 23D, 23E, 23F, 23G, 23H, and 23I, a current user/medical care provider is alerted that a patient has missed appointments with a corresponding user/medical care provider. In another example, a tab to a family doctor 2346 could light up or blink or in any way get a user's attention to indicate that an event is particularly important. In another example and as depicted in FIGS. 23A, 23B, 23C, 23D, 23E, 23F, 23G, 23H, and 23I, when activated by a user/medical care provider, over a blinking endocrinologists tab 2344 can appear an alert window 2316 that can inform a user/medical care provider that a patient has received a diagnosis of cancer. In some embodiments, such important messages can be caused to display without requiring a user to activate or hover over a blinking or colored specialist tab.

There are situations where doctors, even if in separate practices and separate specialties, what they do can impact what another doctor does. By way of example, a retina surgeon injects many times in an eye, up to 12 times a year. But, clearly, if a family doctor discovers cancer that might change the frequency a retina doctor may want to inject. If a patient has a stroke, there are some research studies that suggest the medication that one doctor is using, in this case displayed 2348 injections in the eye, by a retina surgeon might increase the risk of another stroke. In some embodiments, a Rules module of the present principles, such as the Rules module 004 of the Data Command center 001 of FIG. 1, is configured to recognize such situations in which treatment by one doctor can effect a treatment by another doctor and, in such instances, the Rules module 004 is configured to generate an alert to be displayed to all users/medical care providers of such situations.

There are many different ways that embodiments of a medical records dashboard of the present principles can display important information. By way of another example, at any time, if an important event occurs in any encounter of any provider, the information can be inserted into a row in chronological order, where it makes sense, to show on a timeline that the event occurred. So, if it was discovered that the patient had a stroke on May 25, 2019, as depicted by number 2362 in FIGS. 23A, 23B, 23C, 23D, 23E, 23F, 23G, 23H, and 23I the initials of a caring provider can be displayed under the provider instead of a current provider as depicted in FIGS. 23A, 23B, 23C, 23D, 23E, 23F, 23G, 23H, and 23I by 2362 marked as 2364. The difference between providers can be highlighted in many different ways. If it's a provider that is not normally on a row on clinical panel 2380 or for example in this case, illustrated as an example of a retina doctor provider, then this new provider with a row can be highlighted or be a smaller row or a larger row. Also, instead of having the normal information in columns, because the other provider might not perform similar CPTs, instead in some embodiments there can be displayed, at the end of the row in a specially designated area for outside attachments or notes, information and it can be identified if the information is from a different provider.

In the embodiment of the medical records dashboard of FIGS. 23A, 23B, 23C, 23D, 23E, 23F, 23G, 23H, and 23I, 2306 can include financial data, and in this example shows ‘$’ sign. In such embodiments, access to financial data can be limited to only user/medical care providers credentialed to have access for instance only the users/medical care providers and colleagues in their practice can have access. In the embodiment of FIGS. 23A, 23B, 23C, 23D, 23E, 23F, 23G, 23H, and 23I depict, icon 2338 can be activated to enable access to financial data to different users/medical care providers. For example, in FIGS. 23A, 23B, 23C, 23D, 23E, 23F, 23G, 23H, and 23I 2336 is an example of an optometrist and 2338 depicts an icon with appearance of two faces which can represent sharing access.

In the embodiment of the medical records dashboard of FIGS. 23A, 23B, 23C, 23D, 23E, 23F, 23G, 23H, and 23I, the glaucoma specialists 2304 has entry 2306 next to it, which can be used to launch a revenue cycle management (RCM), which is just one mechanism that any user/medical care provider can use to get more information in regards to their own practice's billing or any other information. By way of example, in the embodiment of FIGS. 23A, 23B, 23C, 23D, 23E, 23F, 23G, 23H, and 23I, activating icon 2306 can enable access to a user/medical care provider to cost, charges, any financial information payments, rejections, to which the user/medical care provider has access. In one embodiment, the financial information can comprise a mirror-image of the clinical dashboard, so a doctor, by toggling back and forth, a transparency or overlay can be used to determine what was charged, paid, rejected, or authorized for every service performed. Alternatively or in addition, clicking on RCM 2306 on the same view or on the same scanning screen the information that is financial in nature can be displayed under, over, above, or superimposed, similar to transparent paper, with one embodiment, the billing function, being behind or lighter and clinical being darker or vice versa. In some embodiments, each row of panel 2314 can have 2306 or 2338 next to every one of the tabs (actionable dashboards of different providers).

In some embodiments of the present principles, a user of a medical records dashboard is identified upon use. For example, in some embodiments, a user/medical care provider is required to provide identifying information when the user/medical care provider wants to use a medical records dashboard of the present principles. In some embodiments, a user/medical care provider can provide predetermined configuration information to identify how a medical records dashboard should be displayed for that particular user. For example, in some embodiments a Rules module, such as the Rules module 004 of the Data Command center 001 of FIG. 1 can have access to configuration information for a medical records dashboard provided by a user. In such embodiments, the Rules module 004 can be configured to arrange and cause a display of the medical records dashboard in accordance with the predetermined configuration information provided by the user, for example, upon initiation of the medical records dashboard by the user.

Alternatively or in addition, in some embodiments, a user/medical care provider can drag and drop portions of a medical records dashboard to arrange the medical records dashboard into an arrangement that is best for the user and/or the user's practice or in some embodiments, into an arrangement that is best for a particular patient. For example, an eye doctors might care more about a condition like diabetes, so any doctor that takes care of diabetes, endocrinologists, family doctors, kidney specialists, urologists tend to have more patients and procedures related to diabetes than other specialists, like a radiologist.

In the embodiment of the medical records dashboard of FIGS. 23A, 23B, 23C, 23D, 23E, 23F, 23G, 23H, and 23I, when a user selects entry 2330, window 2374 is displayed for inserting notes, which can then be saved and closed by selecting 2386, or just closed by selecting entry 2388.

Tab 2348 of FIGS. 23A, 23B, 23C, 23D, 23E, 23F, 23G, 23H, and 23I is a tab for providing a user information regarding injections given to a patient, and tab 2366 of FIGS. 23A, 23B, 23C, 23D, 23E, 23F, 23G, 23H, and 23I can provide quick information about the injections including a number of injection or a type of the injections. In FIGS. 23A, 23B, 23C, 23D, 23E, 23F, 23G, 23H, and 23I, 2372 depicts the identification of an example of an Eylea injection having been performed on Jul. 13, 2018, and it is red but can be highlighted in many different ways. In 2372 adjacent to Eylea it says 15 days which in this example count from the last time an injection in the eye was done. In the embodiment of FIGS. 23A, 23B, 23C, 23D, 23E, 23F, 23G, 23H, and 23I, the medical records dashboard depicts that Lucentis was injected 6/18/18 which is only days earlier from a 7/13/18 injection of Eylea and the column counts in the embodiment from one to the other. In some instances, procedures of Eylea or Lucentis injections are allowed only every 28 days from each other. In embodiments of the present principles, a Rules module, such as the Rules module 004 of the Data Command center 001 of FIG. 1, can be configured to have access to information, including but not limited to, rules regarding how frequent or far apart medications can be given, and in some embodiments, the Rules module 004 is configured to cause the display of an alert if a user/medical care provider is attempting to order a procedure improperly or if procedures have already been performed improperly.

In the embodiment of FIGS. 23A, 23B, 23C, 23D, 23E, 23F, 23G, 23H, and 23I, the panel can be used to display diagnostic test and images. In the embodiment of FIGS. 23A, 23B, 23C, 23D, 23E, 23F, 23G, 23H, and 23I, when tab 2350 is selected an interpretation panel 2352 is opened, which can display notes of an interpretation of patient care that could be actually written on the day of treatment. Element 3254 of FIGS. 23A, 23B, 23C, 23D, 23E, 23F, 23G, 23H, and 23I is an image of a test performed on the patient.

In some embodiments, image icons, representative of results of test performed on a patient, can be selected to cause a display of an underlying corresponding image, such that a user/medical care provider can, in context, make a determination of the test and see the actual test while knowing whether there was a procedure or in this example a medication injection done, as depicted in 2366

The embodiment of the medical records dashboard of the present principles of FIGS. 23A, 23B, 23C, 23D, 23E, 23F, 23G, 23H, and 23I illustratively includes a search box 2318. The search box of the medical records dashboard of FIGS. 23A, 23B, 23C, 23D, 23E, 23F, 23G, 23H, and 23I can be used to search for a doctor, a date, an image, particular procedures, a particular diagnosis, payment rejections and payments and substantially any other patient related data/information related to the medical records dashboard. In some embodiment, the medical records dashboard can instantly reconfigure based on what is searched and can be configured to display only the portions of the medical records dashboard for which search results are returned. Combinations of queries can be searched. For instance, show only the rows and dates of service with the diagnosis of diabetes that had injections of a particular medication, column 2348. Instantly, only the rows with injections with the patient having a diagnosis of a certain ICD like diabetes or if comparing a particular diagnostic test with a procedure and trying to correlate it, along with a clinical finding, the user could search “show me only the rows and dates of service where the vision was between 20/20 and 20/80” or “the pressure of 16 to 20 that also had the same date of service, a procedure in 2348 of Eylea and also had an OCT. The patient data/information associated with the medical records dashboard can then be searched and in some embodiments, only rows and columns of the medical records dashboard related to the search can be searched.

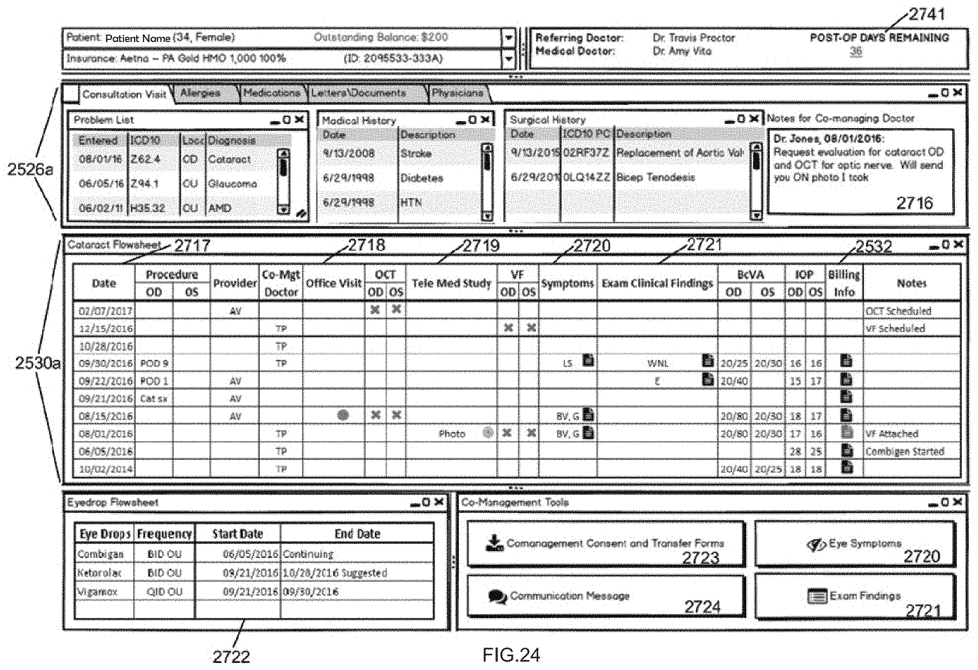

FIG. 24 depicts an embodiment of a co-managed medical records dashboard in the Data Command Center of the present principles in accordance with one embodiment. In the embodiment of FIG. 24, an optometrist and an ophthalmologist share (co-manage) a patient's cataract surgery then share treatment of the patient's glaucoma. A notes field 2716 in the Consultation Visit 2526a panel presents a mechanism to facilitate contextual content surrounding the co-managed procedure(s). A Cataract Flowsheet 2530a (purpose optimized dynamic panel) is presented with structured data elements designed to facilitate the identified procedure as conducted by multiple care givers. The Cataract Flowsheet 2530a (purpose optimized dynamic panel) is presented with structured data elements designed to facilitate the identified procedure as conducted by multiple care givers. The Cataract Flowsheet 2530a is arranged by interaction dates 2717 and tracks office visits 2718 (both scheduled and realized) including sending reminders to patients and alerts when an appointment is missed (not shown), provides means to review and issue concurrence or dissent with diagnostic tests 2719, a summary of symptoms 2720, and a summary of exam findings 2721. The Data Command Center keeps track of appointments between comanaging providers and when an appointment is not kept. The Data Command Center enables messaging to both providers as well as reminders through patient portal for patient to schedule appointment. (describe modules and details of this function). Where available, billing summaries 2532 are presented in the Cataract Flowsheet 2530a as well. Clicking the billing summary 2532 can open a new billing window to show billing details. Eye drops after cataract surgery and/or glaucoma treatment can be tracked on the Eye Drop Flowsheet 2722 (another purpose optimized dynamic panel).

In the embodiment of FIG. 24, there is panel of co-management tools that provide the user with a means to download relevant forms 2723, and to send direct messages to the co-managing physician using button 2724 to access a co-management message center. An indication of the number of postop days remaining 2741 may also be provided. All financial data in the system, including costs to patient, is compartmentalized such that no user can see financial details for users or organizations not authorized in accordance with applicable policies and law. In addition, any rows and columns of information can be programmed to include or exclude those data fields from either provider.

To co-manage a patient using the interface embodiment illustrated in FIG. 24, when a referring medical care provider outside the practice wants a consultation, he or she can connect to the practice they are referring to and send information by opening the Cataract Flowsheet 2530a. The referring physician can manually insert or auto-populate information from any previous visit of the patient and provide an annotation 2717 giving the reason for the consultation. When the receiving consultation medical care provider examines the patient, the Cataract Flowsheet 2530a is auto-populated with the medical care provider's findings. Co-management forms can be downloaded from the table at 2718 either at the time of the referral or after the consulting medical care provider fills out the paperwork and the patient signs a consent form by selecting co-management forms or by visiting the referring medical care provider's website. A message is sent to the referring medical care provider to open the Cataract Flowsheet 2530a and the consent or other forms can be clicked upon and the referring medical care provider can read or sign any forms needed. The “co-management consent” can change color or be distinguished in some other fashion when received back from the referring physician. Every time the surgeon sees the patient, the Cataract Flowsheet 2530a automatically includes the date and findings. Then, post-operatively the co-managing physician, or consultation physician or optometrist, when they see the patient in their office, auto-populates or fills out on the Cataract Flowsheet 2530a and shares any results.

In the embodiment of FIG. 24, notes may be communicated between the medical care provider by selecting “communication message” 2724 to determine if there is any information that needs to be shared for office visits. The date column 2719 and office visit column 2720 are tied together. Some of the columns are left blank until the patient actually shows up for a future visit. For instance, after a surgery or consultation, the consulting medical care provider, just as they would normally give an appointment card to a patient can actually give a co-management medical summary table where it shows the date of the future appointment at the referring or sharing medical care provider's office, and when that date arrives the patient is seen and everything is auto populated so the surgeon can see the results that the co-managing medical care provider found. The findings are auto populated by the optometrist/referring medical care provider/co-managing sharing medical care provider. If the appointment date is missed, the table can link up with the missing ticket report or send an alert to the patient themselves, the surgeon, the referring medical care provider, business managers or anyone else as appropriate.

In accordance with the present principles, shared medical care may be provided in management of common eye conditions besides cataracts, such as glaucoma. For example, an optometrist/general ophthalmologist can manage interval visits after the glaucoma specialist establishes a plan of care. That is, after initial consultation, the plan can be shared with the referring or co-managing medical care provider. At a subsequent examination, the referring medical care provider accesses patient data, executes the plan and enters the data into a Cataract Flowsheet and/or a Glaucoma Flowsheet. An alert can then be sent to the glaucoma specialist confirming that the action plan is being carried out. This facilitates can care for the patient according to the plan. The glaucoma specialist can follow up every year or two while sharing interval visits with the referring optometrist/general ophthalmologist. Multiple benefits of the concepts of the present principles include excellent care, appropriate supervision, reduced cost, improved quality of care of the patient without undue distance traveled. At any point of execution of the treatment plan, treatment can be altered based on clinical data available to the patient, glaucoma specialist as well as the referring medical care provider at all times. Of course, other fields of medicine and industry have similar examples. For example, orthopedic surgeons share care with podiatrists and family physicians share care with all medical specialists. A prime example is shared care with multiple healthcare providers caring for a patient with a chronic disease, state such as diabetes. One patient can have an eye doctor, podiatrist, primary care doctor, endocrinologist, nephrologist, dietician, exercise physiologist, all who need to share care. Different medical care providers can order the same or different tests. If they are in separate health systems, they may not know each other's diagnostic tests, but through the shared medical records dashboard of the present principles, medical care providers can avoid duplication of ordering tests, thereby, reducing costs and delivering better care. In some embodiments, different practices can identify what is important for them to know about a patient and information from the various respective medical records dashboards can be combined so that the identified important information can populate into a single dashboard.

For instance, a general ophthalmologist can have a complex case, for instance neovascular glaucoma, which can sometimes be associated with carotid disease. In some instances the ophthalmologist can send the patient to a glaucoma surgeon. In some embodiments, the pertinent portions of the medical records dashboard of the general ophthalmologist's can be displayed to the glaucoma surgeon, who now has the necessary information to care for the patient. The general ophthalmologist's medical records dashboard can be automatically populated to include the encounters between the patient(s) and the general ophthalmologist, so that medical care providers can, in real time, see what the changes in the patient's treatment are made. In some embodiments, other specialist can become involved in the treatment of a patient and can also have respective medical records dashboards that can share information with some or all of the other medical records dashboards of already involved medical care providers.

In addition, embodiments of the present principles as described above can be implemented to track laboratory tests. For example, every day a family physician and the patients they see can schedule radiological or diagnostic tests to be performed on a patient. A difficulty arises in keeping track of all the different referrals and/or the medications that are prescribed. A medical records dashboard of a Data Command Center of the present principles is able to keep track of every single diagnostic test, medication, or consultation that medical care providers prescribe. Using a medical records dashboard of the present principles, a medical care provider can sort a patient's medical history by date ordered, date performed, or by patient. The results can be automatically collated in rows and columns or in other orientations on a single display. As a patient's laboratory results come back, an entire group of patients that were seen in any time period or for a particular diagnostic test can be displayed in red on a medical records dashboard until the results are received. Upon receiving the test results, the test results can turn another color to indicate the receipt of the results. In such a way, a medical care provider is able to track all of their practice's patients and what the results are, when they are received. In some embodiments, a medical care provider can be alerted to abnormal results.

In embodiments in which the Data Command Center of the present principles, such as the Data Command center 001 of FIG. 1, enables the Co-Management of patient information available via a medical records dashboard of the present principles and as described above, Co-Management is meant to refer to referrals, transfers of care, and any instance of the sharing of patient data either unidirectionally, bidirectionally, or multi-directionally between a Data Command Center of the present principles and any source of patient data and the management of such data via, for example, a medical records dashboard in accordance with the present principles. In some embodiments, a Co-Management process of the present principles can be accessed utilizing a button, keystroke, or series of keystrokes, to initiate the Co-Management workflow.

In some embodiments, upon initiation of a Co-Management process of the present principles, a user can be given the option (i.e., via a prompt on a display) to select a predetermined template for performing Co-Management, to select to determine a custom configuration for performing Co-Management, or to select a hybrid configuration for performing Co-Management. For example, in some embodiments, a template or set of templates can be preconfigured and stored and accessible to at least one of the Rules module 004 and the Display module 006 of the Data Command center 001 for configuring the medical records dashboard and displaying the medical records dashboard in accordance with a selected, preconfigured template. In some embodiments, a predetermined templates can be preconfigured based upon conditions including but not limited to a specialty of at least one medical care provider/user, practice location of at least one medical care provider/user, the identity of at least one medical care provider/user and/or at least one patient, at least one patient's conditions, procedures performed on at least one patient, risk factors for at least one patient, diagnostic results of at least one patient, future orders for at least one patient, future appointments for at least one patient, data values recorded for at least one patient, data values not recorded for at least one patient, calculated data values for at least one patient and absolute values for display. That is in some embodiments, portions, columns, and/or rows of a medical records dashboard to be displayed or hidden can be determined based on a selected preconfigured template of a Co-Management process in accordance with the present principles.

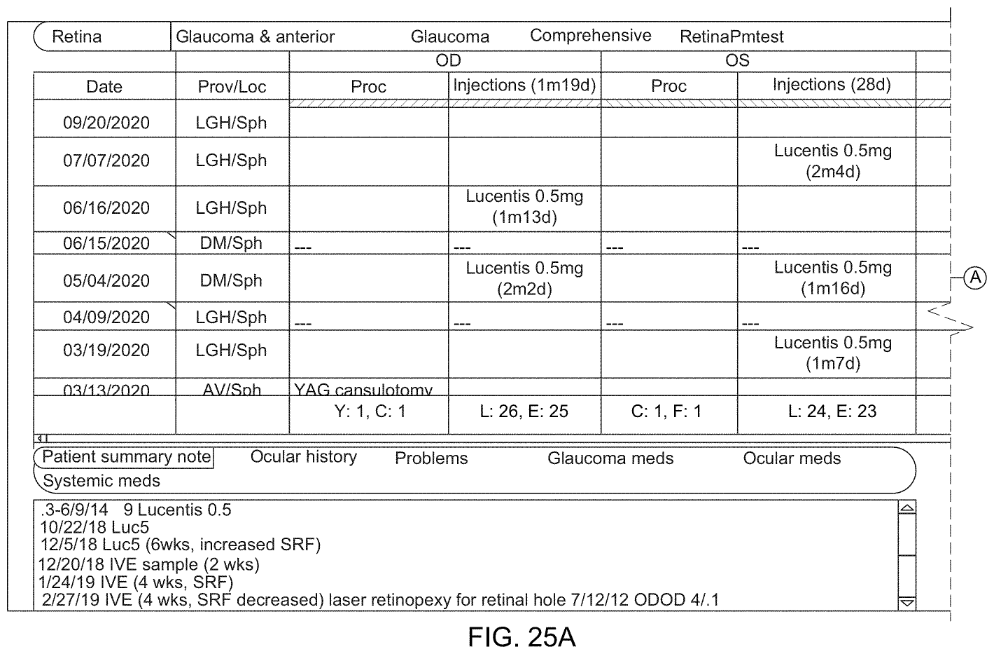

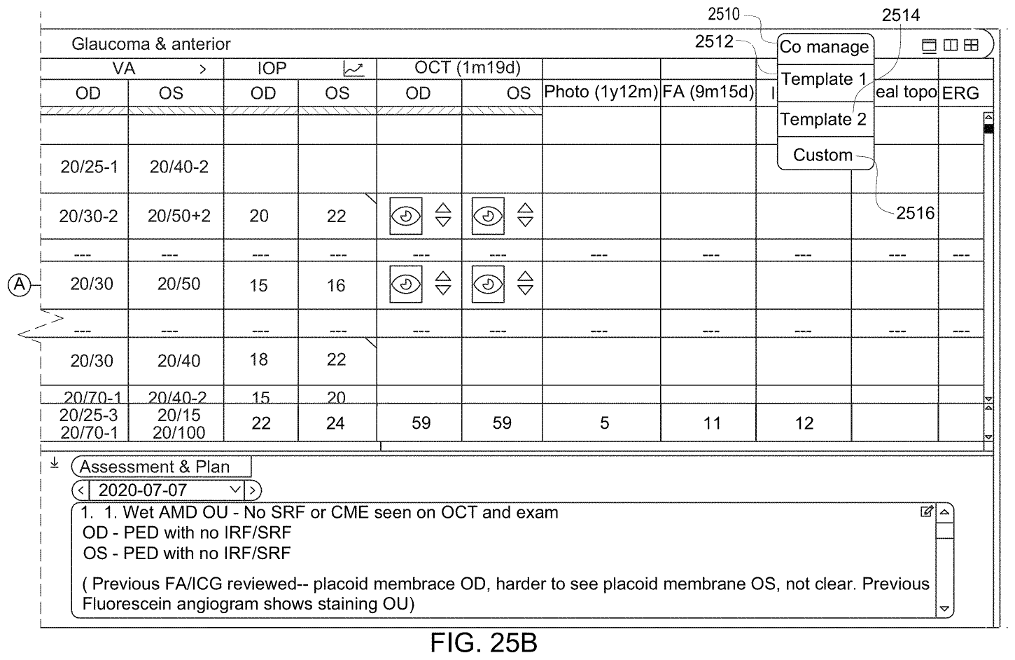

Alternatively or in addition, in some embodiments portions, columns, and/or rows of a medical records dashboard to be displayed or hidden can be determined based on a custom template of a Co-Management process in accordance with the present principles. In some embodiments a Co-management template of the present principles can be determined using, for example, a user interface of the computing device 200 of FIGS. 1 and 2. That is, in some embodiments a user interface can be implemented to create a custom Co-Management template in accordance with the present principles. For example, FIGS. 25A and 25B depict a medical records dashboard including an ability to launch a Co-Management process in accordance with an embodiment the present principles. In the embodiment of FIGS. 25A and 25B, the medical records dashboard includes a Con-Manage icon/button 2510 for launching a Co-Management process. Upon selection of the Co-Manage icon/button 2510, a menu is displayed on the medical records dashboard enabling a user to select between preconfigured templates, illustratively preconfigured template 1, 2512, and preconfigured template 2, 2514. In the embodiment of FIGS. 25A and 25B, the displayed menu further enables a user to select the ability to create a custom template 2516.

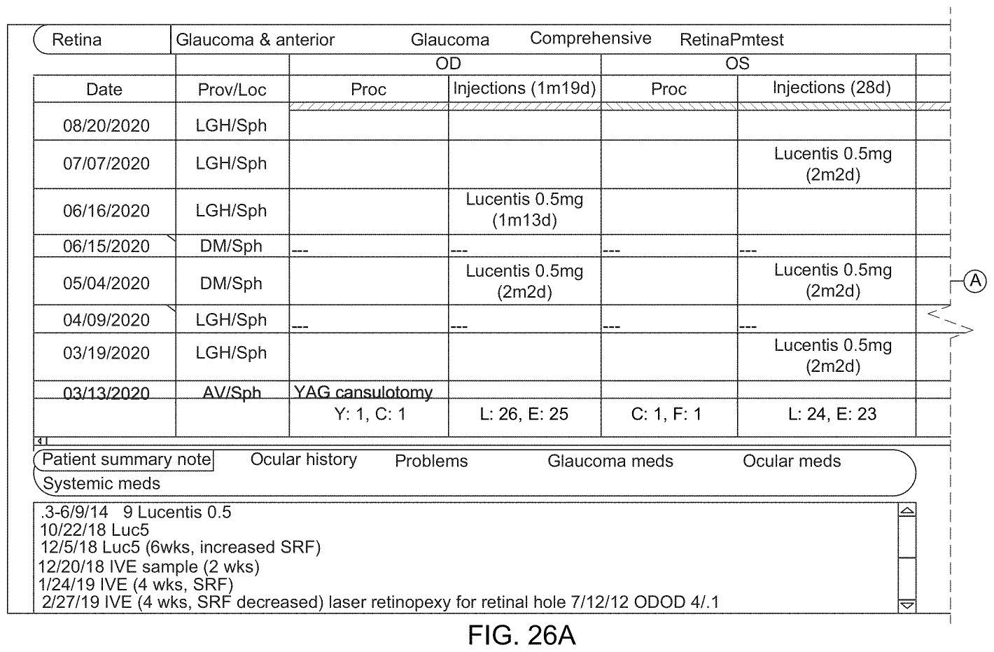

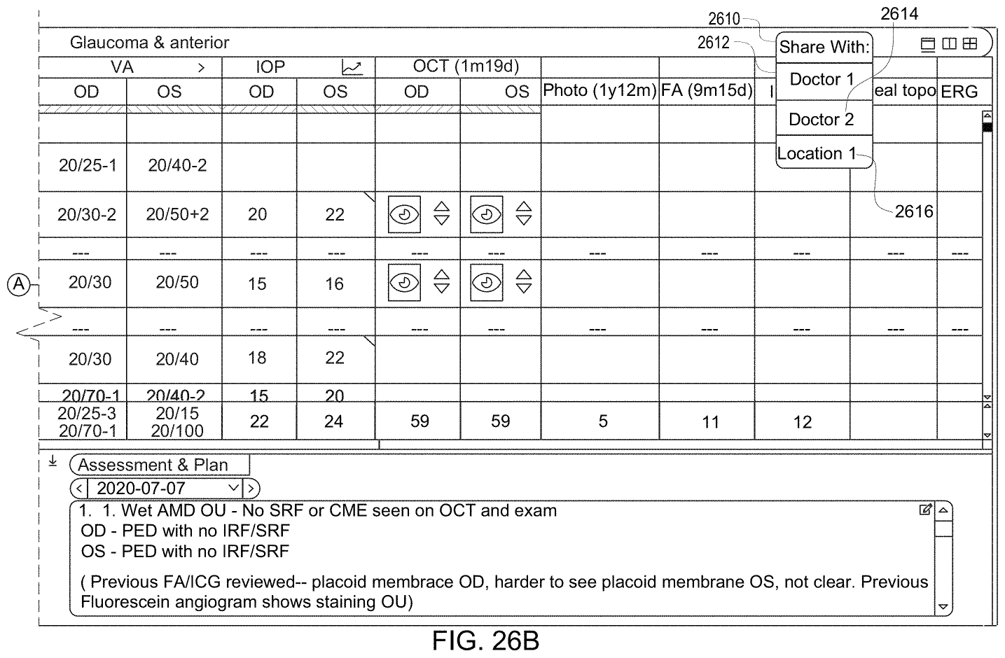

Upon selection by a user of the custom template 2516, a process is initiated that, in some embodiments, enables a user to select portions, columns and/or rows of the medical records dashboard to display or hide. For example, FIG. 26 depicts a medical records dashboard including a custom template creation process for co-management in accordance with an embodiment of the present principles. In the embodiment depicted in FIG. 26, a user is given the ability to select, for example using a user interface (i.e., mouse, keyboard, etc.), portions, columns, and/or rows of the medical records dashboard to be accessible to (i.e., displayed to) a shared user(s) of a Data Command Center of the present principles, such as the Data Command center 001 depicted in FIG. 1, via a medical records dashboard in accordance with an embodiment of the present principles. Alternatively, a user can select, via the process described above, portions, columns, and/or rows of the medical records dashboard to be hidden from (i.e., not displayed to) a shared user(s) of the Data Command Center.

The ability to select options for displaying rows and columns may be provided in a table format in one embodiment. Options for display of data in row selections may include text describing the options with a check boxes to enable user selection as desired. Some example options include:

Options specified above, and further options below enable a user to set the particular parameters for the rows (or columns that will best help them accomplish their particular job function and to integrate the encounters from those they need dates or time of encounters. The options are numbered and will be referred to in this text with a “#” preceding the corresponding number. The options allow a team working together to efficiently and accurately accomplish their individual and combined goals. It is for the user to choose their options, by first interacting and selecting options shown above, and as so doing then interacting next with options described below to complete the selection and in some cases, additional options are presented dependent on the number of options users chooses to have or the complexity of situation chosen. For instance, if there are numerous ICD 10 or CPT codes that can be chosen. This is just one example of an embodiment of the tool explaining the overall function and the choices are adjustable for each different type of field and user. Once the process of clicking options above and below and any associated drop-down options and panels to confirm the user selected, what in fact was desired and try to avoid an accidental wrong click.

#200-206 demonstrate a confirmatory process within #200 allowing the user to actually visualize what they have selected as the options, and it sets the parameter.

#1: The row selection that enables the user to select which line or row, in one direction, is chosen. Then, once clicked, the decision then needs to be made as to how it should be displayed, so the user is taken to #2 or the options below.

The process is: First you click #1, then you look to see which item you wish to select next. For instance, #3, 4, 5, 6. Once making the selection then any sub-selection that drop-down. For instance, #7, 8, 9, 10 and if there is any other drop-down menu necessary to be more specific to select, for instance under #8, which would be a particular office, if there is a certain sides to the office or other decisions to be made within a very large complex, you would click #11. It would also come up automatically more drop-down options if programmed. This process is repeated for all of the numbers. Once you have completed the selection in #1, you then go over to #2 and make your selection in that panel on how you want what you selected in #1 to look.

After selecting #1, you can decide how, if selecting #3, all the dates which are essentially in time and date order in one direction. For instance, rows would be chronological and would be the major selection. You can go from top to bottom, oldest or newest (the tool also provides for a sorter function, so anything can be asked and only those rows that meet the criteria of the question are displayed or just those rows can be highlighted temporarily or permanently. This process and option allows a user to synthesize and analyze data in a unique way going way beyond spread sheets that just summarize, add, and subtract particular columns or parts of a column, and this tool allows direct visualization and highlighting that helps the user for instance find patterns).

#4 defines a time period that you want the tool or dashboard to display. Is it from the beginning of time that is recorded and there is data? If so, the user selects #4A. So, you choose all or is it from a certain period of time #4B and you can select in #4C a beginning then in #4D a conclusion. This is a typical method of a calendar coming up where you can select a day, month, or year or it can be typed in or it can be any mechanism to help the user to insert a date or time, but all options can be allowed in this spreadsheet. Even dictating it and speaking through voice recognition.

#5: Shows offices and if that is selected, the user is choosing a location. The location can be where they work or all offices of a practice. This can be customized to each entity that uses the tool. This example #7 shows the selection “all offices.” You can select #8 and choose just one office or additional offices #9 and #10. The other offices, some users may have just two offices, others might have 30. After selecting a particular office, #11 shows an example of where there can be another panel that would open up within an office. There could be many more options. If it is a hospital system using this tool, #11 could be very extensive because it might be floors, departments, or even multiple locations within a hospital in one particular geographic radius.

#6: Numerous providers can be selected. #12 allows you to select which time period the spreadsheet should populate, which could be from the beginning that the patient has been in the practice starting in the practice, which is likely most common, so if all #12 is selected and #13 allows entry of a particular user, could be the signed in user, so tool would, if #13 is selected, populates this particular doctor. Then, how that doctor wants their personal row displayed is chosen in the options below. Next #14 allows the choice of populating the spreadsheet with more encounters from other providers and user selects as many as desired. If the choice of time period to populate is different then, #16 allows, a specific time period, and #17 allows that time period to be for just certain doctor or #18 allows you to select from many providers, by specialization after you have selected. When selecting #12 explains all time periods from the beginning when the patient started to whatever is in the data to the future. #16 allows you to specify a specific time periods of day, month, year or it could be time in a day. As in a hospital admission, patients can be examined 24 hours in a day. There could be 24 rows. If a scientist is doing research a second or microsecond may be chosen as the time element or astronomers may choose time in 1000 year increments.

Clicking #13 can know this is being set for just one particular doctor or provider. If it is not just one particular provider #13 lists just the doctor or provider and a drop-down menu can occur with many different providers.

#14: Allows you to select exactly which providers that are using the table. #15 gives an example of A but this can be as many providers as necessary. You can first click one or several or chose them all. It all depends on who is using the tool and who is setting it. If it is just one doctor setting for himself or a group of doctors, all getting together to set, what they all want to see that is one way the tool can be easily set. But, setting for a large health system or corporation with many thousands of employees takes more consideration. Who the providers are and how they are categorized can, in this large corporate or even international corporate entity, be very complex. The tool accommodates all options from small personalized organization to large complex international entity are entirely different.

#16: Again, allows you to select a specific time period. The day, month, or year. When selecting again #17 can be specified for which doctors. #18 again, selecting a group of doctors. #12 is all time periods. #16 you are actually specifying what time period the tool is sorting, selecting and presenting the rows and options below allows the option of how it is to be displayed.

#19: Is by specialization. #14 is more particular doctors chosen, but #19 is grouping of doctors. When choosing a group there are examples of just a few that the tool would know are specialization. By way of example, if the specializations is just eye doctors, the tool would then prompt options A, B, C, D, E, and F choices of eye doctors. #20 being retina, #22 glaucoma, #24 optometrists, all types of eye doctors, and so on and so forth. If it is for a large health system or otherwise, where there are many different options. All options #19, A, B, C, D, E, F and so on can be any type of specialist. Orthopedic surgeons, dermatologists, or if it is outside of medicine, it could be different types of lawyers in a law firm. Those handling civil cases, divorce, real estate, patent, business, corporate, etc.

#26: Allows for missed appointments. This is more of a catch-phrase term. If it was an appointment that was supposed to occur for whatever reason is displayed and even the reason could come up by hovering or other means, using the example of a patient. The next section would be giving details.

27: When hitting details there can be many different reasons that can roll over or other reasons or display in different ways. For instance, #27A is cancelled by patient. #27B is cancelled by practice. #27C is a no show. All of these might have different reasons. If a patient continues to no show for an appointment, instead of cancelling in advance, that might want to be displayed differently, practices cannot fill scheduled slots if patients do not cancel in advance. How it would be displayed is selected in #2. But, how it is categorized and show up as a row is determined by the checked boxes.

#28: Selected if the rows should only be within the practice and were selected above. For instance, #30 shows providers outside the particular practice using or setting the spreadsheet as shown in the options above and below. If you are only interested in your own legal firm, accounting firm and following on a table what you personally have done and sub dividing all the different lawyers working together in your firm, do you really care what lawyers on the outside do? Also, when those outside entities saw a client, does your firm care-perhaps or perhaps not, but the tool allows the user to select that choice. In a field like medicine this could be critical to see encounters outside the users own interaction with the patient. While the practitioner might not work within your entity, a patients heal this interrelated and even if the patient always comes to your large health system, what if the patient happens to visit, somewhere 1000 miles away, and has a major medical event, even though the provider is not within your practice, of course a doctor would need to know because when the patient comes back, it might affect their treatment of that patient. Even if in the same city, the patient goes to different hospitals and different doctors, all can be informed as to what the other provider does or discovers about a patient, as they may be inter-related. Therefore, certain users of this tool might want to see all of a sub-set of the different providers, even if they are not related to the direct practice.

#32: Allows you to select a particular provider.

#34: Allows selection of a certain specialty.

#36: Can be any category. User needs to designate for them to maximize use of the tool.

#38: Can allow selection of all the providers in a certain practice or entity, A, B, or C are examples of entities outside user's own practice, but no matter what provider is working in the particular practice A, B, C, or D, they may want to see certain rows of other users selected.

#40: Hospital admissions. Is it to be included? Could also be emergency room or any type of sites or issue. User setting the criteria or other methods in other embodiments would respond yes or no in A and B. Again, how this is displayed each time would take you to the below options, how to display for selected rows.

#42: Question is, do you want to share care with the above? Here you say, yes or no. #42 D allows you to click and you can choose particular columns to be shared. This takes you to an entirely new panel (not depicted), which goes into great detail to choose what information to share between two entities that may be sharing the same tool. For instance, they may have a dashboard, spread sheet and a second practice that is filling and selecting the usage of the tool may also have a spread sheet. Both can share those spread sheets, both can insert rows into each other, but which rows are going to be shared and how are they going to be displayed would be #2. But, here D represents a whole other panel that comes up. This would require both confirming they will accept the others rows and columns or perhaps only allow read only access or just one user accessing the others. The analogy is similar to the need to opening both doors in two adjoining hotel rooms to allow entry for both. Only certain rows or columns can be selected to be shared. In this case, since only one provider is filling this out and allowing certain information to come over into their spread sheet. The reverse is not true, unless it has been arranged to be that way and the other user allows this. When there are the shared rows or spreadsheets and depending on the industry regulations, would be followed with necessary permissions obtained and confirmed by the tool before allowing entry. Example in health care, it would require patient record release and sharing approval with HIPAA rules followed.

#44: Allows, if doctors communicate on the phone, telemedicine or any which way that, or a provider talks to another provider at a certain time and wants to leave a row for when that happens, and what was said can be submitted there as a row.

#46: Patient phone call encounter. In medicine there are usually only rows for actions when the doctor sees the patient or performs an examination. In reality, currently every time a patient makes contact, and asks a question about a symptom or a problem, doctors do not even know the call occurred unless they are contacted and something important can easily be missed. Same with #47. There could be a message through the portal. When the patient had the symptom or the question can be important. This tool allows an option #46 and #47 to insert in a row. #49, if the office contacts the patient, that might need to have a row. But, how that is displayed, of course, if very different than any other row. That is why after hitting each of the row selection columns, you are brought over how to display the selected rows in the options below. In a case like this #46 and #47 might not be always a relevant as #13. For example, the user might choose to hide, make collapsible, barely visible, or just a line with color. Knowing that there is something there and it can be hover over and expanded is maybe what is important.

#47: The user can select in #47, the monitoring device, which sends information results from any entity. An extensive drop-down menu is presented as monitoring or diagnosing can mean virtually anything and the user can decide what is important for them to display. One embodiment actually captures the ability of the computer to help select what's important in a particular row, based on how the doctor has told the system they want information to be displayed. It does so which can occur without human interaction. In some cases, if the user has selected, in the system, to not include certain rows of certain providers, there are certain programs and circumstances that are informative and there are certain programmed circumstances where the information still may be displayed because somethings the information is so urgent can also be allowed.

Spreadsheets usually require significant human entry and interaction. This tool is significantly autopopulated, although it can be manually populated. But, no matter how data is entered, whether automated by computer or human interaction and entry, what has been displayed and how time and date is presented has equal presentation as mentioned above. This tool can measure critical events even if it's not in an encounter of the user itself. For instance, if the user wants to see laboratory results, pathology specimens, radiological studies, many of these require human intervention to interpret. Yet, it's lost somewhere in the computer. This tool can automatically timestamp when the result or event occurs. It can put in a row, as designated by the user, how it should look in chronological order or any other matter within the dashboard. These outside events can be on a different portion of the dashboard not necessarily chronological, but it is up to the user and the way it's displayed, which impacts differently, so it catches the user's attention until it's looked at and opened. The most common embodiment has the presentation in the rows when the results come back or when the test is performed. So, while the rows can just represent user interaction of health care practitioners to a patient, they can just as easily be the result

#48: Brings up a drop-down menu. This represents monitoring techniques or devices and sending information results from any entity. In all fields, there can be incredibly important attachments or events that may be unrelated to a particular time that the user actually interfaces with the client or in the case of health care with the patient. Doctors may place orders and the orders for certain diagnostic tests to discover disease, such as radiological studies, blood tests, etc. could be done alone on an entirely different day than a normal row, which has been selected. For instance, in #13, and #14, when the user actually interfaced or took an action, with or without a client, or in this case a patient, the test or any other means of gathering information may be completed on a certain day or time. To make certain this is not missed, it could be organized in many different ways. It can be set to be attached to the rows the next time a user sees the client or patient or it can be inserted chronologically when completed. By way of example, if a doctor sees a patient in September of 2019 and schedules the patient, again, one month later, in November 2019 but orders three different tests that are done on three different days and the studies come back in October, all three dates in October that they were either performed or the results come back can be inserted in a row. Then, when the doctor sees the patient in November, they will see the rows that are displayed, which will be selected from options below. It can be clicked on, get the results, and then turn off what had been an alert that was set in the options below to highlight more significantly. Once read, it perhaps becomes less significant by hitting another display option below.

#48A is an example of diagnostic tests. #48B could be pathology reports, and #48C could be major life events of any kind. In reality, especially in a field of healthcare, if a patient has a sudden death of a loved one, this can impact everything else. The date of that death, because of a sudden car accident, could impact the health of the patient in every way. Therefore, knowing the date, by putting it in a row, whether manually or automatically, if it happens to be an event that can be collected automatically, if not put in manually, may impact other dates of service and other treatments or plans of action going forward. #48D gives an example of home monitoring. In the field of medicine, patients are now able to check their blood pressure or other items at home. If there is a major occurrence while measuring blood pressure that is out of the normal standard for that patient, that high or low event could be placed in a row so when the doctor interfaces with the spreadsheet they can see what happened. Clearly, how this role presents needs to be very different then a major surgery event or a major encounter by a doctor. Hence, why this tool and only the stool allows the user to differentiate items in time and date as shown in the options below.

Once the selections are completed in #1, it immediately takes you one by one to the how to display for selected rows, discussing this in depth. Once something is selected in #1 you can make the selection of #70. It will select #70 and the issue is one time only so you are making a particular selection on a particular row in this case and it is only one time. Perhaps it is just a certain day, month, or year #72. It is a time period from a certain day, month, or year #74. Display until the patient returns for the next visit, so it is only a temporary situation. But, you can choose the temporary situation being the treating provider, which could be the primary provider, #13 that modified the tool for their use. When the patient returns only to that provider, that could be an option to only display until that provider (#76) opens or sees the encounter or #78 could be kept open until any provider in the practice next encounters the patient so, the row remains highlighted as chosen below until then. #79 allows it to remain open and not disappear until a doctor clicks it and it is certain it has been seen and only then highlighted method chosen turns off.

#200 and #202: Allow confirmation of any step or selection.

Options for how to display selected rows are now shown:

#80: Allows whatever had been selected in #1 to be permanently displayed.

#82: Allows just a certain time period for it to be displayed. The beginning and the end can be selected.

#79: Keeps displaying it until it is opened by a provider. That way, whatever, was the important row that was submitted and inserted has to be opened by someone. It can even be made permanent at that time or who opens it might matter.

#80 is a permanent selection for the rows. #82 is just taking the action for certain time period.

#84: Selecting this option sets whatever was selected in #1 is inserted in rows at the normal size, which is the most common choice, or instance, #13 or #14. But, when doing special considerations, for instance #30, and #46 or other examples, clearly that needs to be differentiated and the user would be unlikely to choose #84. That is where the specialization selection process that follows will come into effect. #86 allows any row or group of rows, as defined, to be smaller any size can be chosen. It can be half size, one third size or other.

#88 allows it to be a larger row, two times or three times. Also, any of these rows can be from a certain time period. Option #79 can keep it that size until it is open. The concept is something needs to be larger because it is important one time until red or acted upon, then it can go back to a different size. When opening any row at one time, that particular row can be adjusted at the time it is seen and acted upon. #88 can enlarge the column and in all cases the time period and which rows are to be acted upon are selected.

#89: Strike out so it is dashed in between the line.

#90: Choose any color, as seen for instance as it is normally found: Red, yellow or other.

#91: Make previously selected rows defined and selected above bold. That is true with #70 through #103.

#92: Lighten or have row fade out.

#93: Hide

#94: Collapse

#95: Move

#96: Make editable. This is a very important feature. It allows the user to actually edit a row and save it and be able to write anything in this row by clicking option. This takes you to an entirely other option where how you make something editable, what you can do, what you can save, how to save it, where you can copy from other areas to put something into this row can all be achieved.

#97: Allows ordering panel access. This means that any particular row or a cell within the row, as an intersection in a column could allow and activate a mechanism to open up an ordering panel that would allow a user to make an order and allow for other functions to occur because something happened in the row. This, again, when you hit #110 would take the user to an entirely new panel that will allow for the types of ordering and options and what type of options to create in the row. By way of example, if the row happens to display a diagnostic test or does not display a diagnostic test, because it is lacking an order for a future diagnostic test could be started by hitting this particular row.

#98: Allows for action to be done with one click on anything on that row and that can be set.

#99: Allows the mechanism to pull up data that is found in a row. This could be anything from a diagnostic test or an image to something written or more information and numbers.

#100: Allows a roll-over mechanism. On that row, there can be more information and simply rolling-over or any aspect of the row and/or column or intersection would pop something up.

#101: Pulls up a window mechanism as well as an option for a link within that row for anything that is set in that row. This can take the user from one row to another location to gain more information on a subject matter on the row.

#102: This will allow changes in a row from the original presentation or selection, so when clicked, that particular row can be manipulated any which way even overriding what had originally been set. Only certain users can do this and it may just be user based because on user can make a choice of a change in one row that other users, who use the same dashboard, might not agree with.

#103: Choose any option #85 to 95. This is done because if you choose #102 where you can make changes in any row, you might want to highlight that row in some way. It will then, allow you to make an option by choosing #103. Any option you want.

#103: You select here for the choices of how to select the columns.

#110: Allows the user to make selections and changes for the other direction in the dashboard. For instance, the columns. To start the action, hit #111.

#112: You are making these options more permanent.

#113: Only when integrating from outside of the practice, the columns might be selected. It is important to note that this particular option to repeat for the columns for whatever the other direction is not being expounded upon because dashboards do have many options that are in existence to choose in one direction only how a column should look (but they do not have options to do the other direction). This tool allows the user to, in one direction, perhaps columns make the typical changes that allow insertion of columns on a dashboard. This tool allows the other direction which are not set to be set.

In some embodiments in which a user selects to create a custom template, upon selection of the creation of a custom template, the Rules module 004 can initiate a process, for example as described above with reference to FIG. 26, for enabling a user(s) to select to which to portions, columns, and/or rows of the medical records dashboard a user(s) is to be allowed or denied access. In some embodiments the Rules module 004 stores such custom template configuration selected by the user(s) in a storage means accessible to the Rules module 004 and the Display module 006. Upon creation of a custom template for Co-Management in accordance with the present principles, the Display module 006 can cause the display or lack of display of portions, columns, and/or rows of the medical records dashboard based on the determinations and information associated with a created, custom template.

In addition to the selection of a preconfigured template, for example preconfigured template 1, 2512, and preconfigured template 2, 2514, and/or the creation of a custom template, for example custom template 8716, in some embodiments, a Data Command Center of the present principles, such as the Data Command Center 100 depicted in FIG. 1, via a medical records dashboard, can enable a user(s) to select parameters that decide to whom/what/where to enable access or deny access to portions, columns, and/or rows of the medical records dashboard. For example, in the embodiment of FIG. 26 the medical records dashboard comprises a Share With menu 2610 enabling a user(s) to select to whom/what to enable access or deny access to portions, columns, and/or rows of the medical records dashboard. In some embodiments, the Share With menu 2610 can include predetermined selections such as a first doctor, Doctor 1 2612, a second doctor, Doctor 2 2614, and a location, such as the location of a medical practice, Location 1 2616. Alternatively or in addition, in some embodiments the medical records dashboard can enable a user(s) to input identifying information including but not limited to a specialty of at least one medical care provider/user, practice location of at least one medical care provider/user, the identity of at least one medical care provider/user and/or at least one patient, at least one patient's conditions, procedures performed on at least one patient, risk factors for at least one patient, diagnostic results of at least one patient, future orders for at least one patient, future appointments for at least one patient, data values recorded for at least one patient, data values not recorded for at least one patient, calculated data values for at least one patient and absolute values for display to identify to whom/what to enable access or deny access to portions, columns, and/or rows of the medical records dashboard.

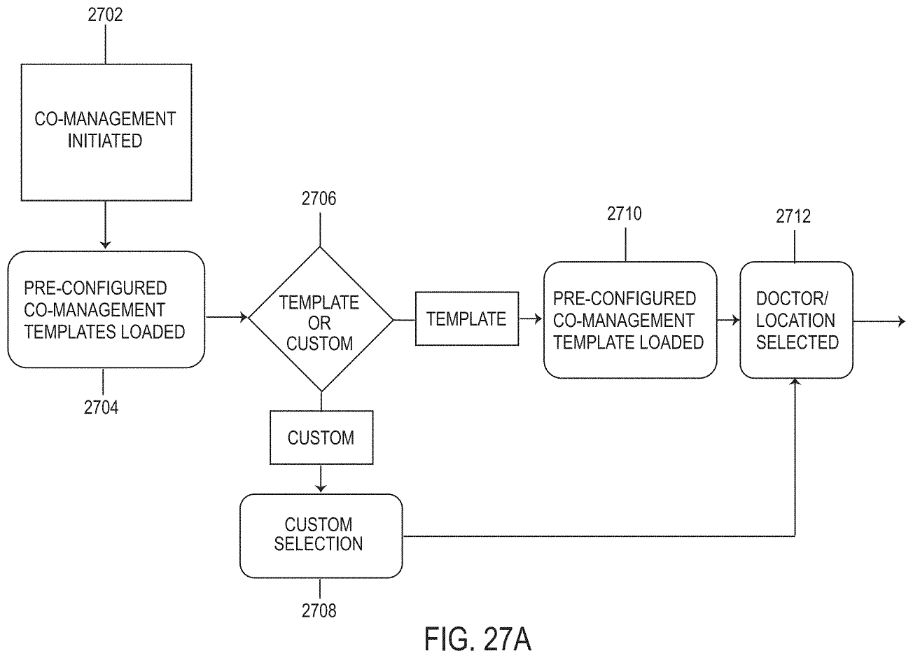

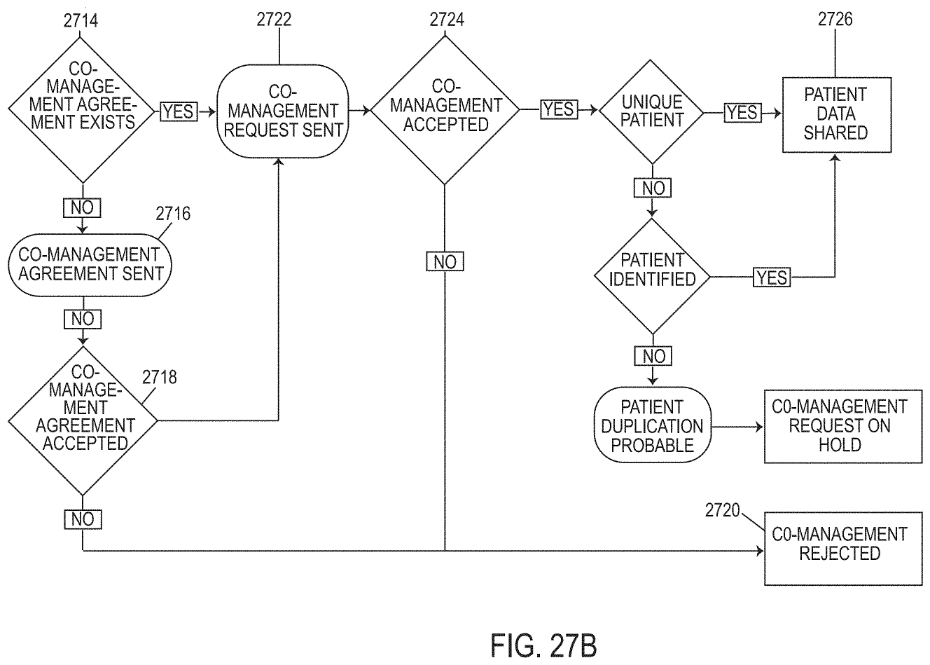

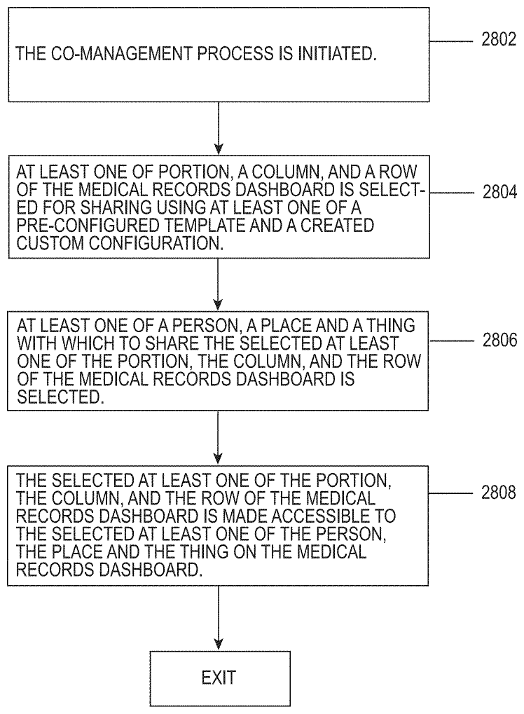

FIG. 27A depicts a first portion of a workflow diagram of a Co-Management process and FIG. 27B depicts a second portion of the workflow diagram of the Co Management process of FIG. 27A in accordance with an embodiment of the present principles (referred to collectively as FIG. 27 herein). In the embodiment of FIG. 27A and FIG. 27B, the Co-Management process is initiated at 2702. At 2704 preconfigured Co-Management templates are all loaded. A selection is then made by a user(s) to use a pre-configured template(s) or to use the Custom option to create a Custom Co-Management configuration at 2706. If a user selects to use a pre-configured template, the selected pre-configured template is loaded at 2708. If a user selects to create a Custom Co-Management configuration, user selections for creating the Custom Co-Management configuration and determining which portions, columns, and/or rows of the medical records dashboard to which to grant or deny access are made at 2710. In the embodiment of FIG. 27A and FIG. 27B, at 2712, a user selects select to whom/what/where to enable access or deny access to portions, columns, and/or rows of the medical records dashboard.

At 2714 it is determined if a Co-Management agreement exists. If no Co-Management agreement exists a Co-Management agreement is communicated to at least one other user at 2716. At 2718 it is determined if the communicated Co-Management agreement was accepted by another user. If the communicated Co-Management agreement was not accepted by another user, the Co-Management agreement is cancelled at 2720. If at 2718 it is determined that the communicated Co-Management agreement was accepted by at least one other user, a Co-Management request is communicated to an accepting user at 2722.

Referring back to 2714, if it is determined that a Co-Management agreement does exist, the process also proceeds to 2722 during which a Co-Management request is communicated to at least one user with which the Co-Management agreement exists. At 2724 it is determined if the Co-Management request was accepted. If at 2722 it is determined that the Co-Management agreement is not accepted, the Co-Management agreement is cancelled at 2720. If at 2722 it is determined that the Co-Management request has been accepted by at least one user, the patient data is shared at 2726 in the medical records dashboard in accordance with the pre-configured template selected or the custom configuration created and the whom/what/where selections made by a user(s).

FIG. 28 depicts a flow diagram of a method for Co-Management of patient information in a medical records dashboard in accordance with an embodiment of the present principles. In the embodiment of FIG. 28, the method begins at 2802 during which the Co-Management process is initiated. For example and as described above, in some embodiments the medical records dashboard can include a Co-Management icon/button for initiating a Co-Management process in accordance with the present principles. The method can proceed to 2804.

At 2804, at least one of a portion, a column, and a row of the medical records dashboard is selected for sharing using at least one of a pre-configured template and a created custom configuration. The method can proceed to 2806.

At 2806, at least one of a person, a place and a thing with which to share the selected at least one of the portion, the column, and the row of the medical records dashboard is selected.

At 2808, the selected at least one of the portion, the column, and the row of the medical records dashboard is made accessible to the selected at least one of the person, the place and the thing on the medical records dashboard. The method can then be exited.

In some embodiments, the Co-Management Workflow can exist in a single, unidirectional state, whereby the party that initiates the Co-Management request shares data with the recipient, but the recipient does not reciprocate sharing of patient data. In another embodiment, the party that initiates the Co-Management request shares patient data with the recipient, and the recipient initiates a Co-Management request to the party that initiated the initial request, thus data is shared bidirectionally. In another embodiment, several parties initiate Co-Management requests, and each party shares data with each other party, in a multi-directional state. At any point, a Co-Management participant my opt to no longer share data with one or more recipients, at which point data sharing and the Co-Management workflow reaches a logical end.

In some embodiments, upon initiation of Co-Management, a record of the Co-Managed patient is recorded, including all relevant Patient Identifiers from all parties involved in Co-Management. Alternatively or in addition, upon initiation of Co-Management, shared configurations are recorded. Shared configurations can be used to determine what data from each party can be viewed within a recipient's medical records dashboard in accordance with the present principles.

In some embodiments, a source of patient data can exist within storage means associated with respective Data Command Centers of users participating in the Co-Management of the present principles. In such embodiments, shared data can consist of a series of links or cached data in the respective Co-Management databases. Links or cached data can be updated upon any change in source. Additionally in some embodiments, data can be recorded within a Co-Management database as well as a database/storage means associated with a participating user's respective Data Command Center, the data including, but not limited to, audit logs of Co-Management Workflow interactions, Messaging between users, file and document sharing between users, and notifications and/or triggers for automated tasks. It should be noted that, in some embodiments, a Co-Management Workflow in accordance with the present principles can be non-linear, can be automated in whole or in individual or groups of steps, and algorithms can intelligently update, flag, or otherwise override certain steps of the Co-Management Workflow.

In one example of a Co-Management Workflow in accordance with the present principles, a primary care physician (PCP) can initiate the Co-Management Workflow for a single patient having multiple Specialists. Each Co-Managing Specialist can opt to Co-Manage with one of more PCPs and Specialists. In some embodiments, the Co-Managed patient data would not be shared further than one logical step, thus a PCP can share their patient data with Specialist 1, who then shares their patient data with Specialist 2, but the PCP's patient data would not be shared with Specialist 2 unless the PCP takes action to initiate Co-Management with Specialist 2.

In a second example, a doctor can initiate a Co-Management Workflow of the present principles with a patient during a Transfer of Care, in which case, the patient's data is shared unidirectionally, and the recipient is not expected to share data back with the initiating doctor, nor is there an expectation that the patient would return to the transferring doctor.

In a third example of a Co-Management Workflow of the present principles and with the context of a hospital and several physicians, as is normally the case in patient care, any number of Co-Management Agreements and Workflows can be in place to allow for patient data sharing between any to all recipients of a Co-Management Request. This configuration can include unidirectional sharing, bidirectional sharing, and multi-directional sharing of patient data in accordance with the present principles.

In co-management, where different practices share information about the same patient, it is critical to identify that the patient that is being shared is in fact the same person. There can be dozens of John Smiths and systems cross-reference by looking at the last name, the age, the gender, the zip code and perhaps the home address. But still, there can be confusion between patients. In medicine you can take no chances that you confuse one patient with the other and when patients travel from different offices or different EMRs and computer systems, the possibility of confusion is present.

In some embodiments, the Data Command Center of the present principles, such as the Data Command center 001 of FIG. 1, enables unique patient identification by incorporating patient medical history information. Current methods for identifying patients include matching Social Security Numbers (SSN) and Driver's License Numbers, where available. However, as privacy became more of a concern in the modern digital age, such data is becoming less available to medical care providers and their Practices. In addition, other methods for identifying patients can include identifying patients via First Name, Middle Name or Initial, Last Name, Age, Sex, Address, City, State, and Zip Code. Such information, however, is subject to flaws of human error, such as typos, human choice, such as a patient offering a nickname instead of the accurate name on a birth certificate or other identification. In addition, even having accurate patient information, it can still be difficult to distinguish between two people having the same name. Using such current methods, multiple systems are only able to match patients whose information is listed exactly the same in the multiple systems, a limitation which requires human intervention and prevents full automation of the process.

A subset of data exists within the Medical Community, as mandated by Meaningful Use 2014 and 2015 EHR Certification requirements specified in 45 CFR § 170.102, known as the Common Clinical Data Set (CCDS). The CCDS consists of patient information including, Patient Name, Sex, Date of birth, Race, Ethnicity, Preferred language, Smoking status, Medical Problems, Medications being taken, Medication allergies, Laboratory test(s) having been performed on the patient, values of the Laboratory result(s), Vital signs, Procedures, Care team member(s), Immunizations, Unique device identifier(s) for a patient's implantable device(s), Assessment and plan of treatment, Treatment Goals, Health concerns and the like.

CCDS was developed to encourage interoperability through the exchange of a common data set and is routinely shared between practices by means of the Direct Messaging Exchange, a secure messaging system by which Continuity of Care Document (CCD) or other document conforming to the Clinical Document Architecture (CDA) as defined in the 2014 and 2015 Certified EHR requirements. This is the current standard for Clinical Data transport between EHRs, thus between practices. The future requirement, Fast Healthcare Interoperability Resources (FHIR), expands on the clinical data set to include more discrete data points.

In accordance the present principles, the inventors propose to incorporate such additional data, such as the data supplied through the CCDS, to accurately identify unique patients using a combination of techniques including but not limited to a Common PII Matching technique, a Problems, Allergies, and Medications technique, a Doctors, Locations, and Procedures technique, and CCDS data technique.

In a Common PII Matching technique, none of the PII data may be valid given name changes, nicknames, and misspellings, as well as marriage and legal name changes, addresses and phone numbers change over time, and the increasing reluctance of patient and practice alike to maintain or share key identification numbers. At best, every data point would need to match exactly to ensure the closest match, but can still fall short in the cases of same names such as in the case of George Forman's eight sons all named George Edward Foreman, if date of birth and suffix data was not present. Twins could make identification even more difficult. As evident, the Common PII Matching technique may not be reliable on its own for identifying unique patients.

In a Problems, Allergies, and Medications technique, a commonly shared data set which includes key conditions (Problems), allergies to certain medicines (Allergies), and specific medications (Medications), is compared to determine a profile of a patient which offers an additional level of accuracy by taking a loose match from PII and determining if that patient also has the same list of Medical Problems, Allergies, and Medications in a system for comparison. The likelihood that two people within similar PII, or lacking key aspects of PII, would also share the same Problems, Allergies, and Medications is a significant reduction in ambiguity. For instance, George Foreman's 3rd son may share certain genetic predispositions to Medical Problems and even share Allergies with a 1^{st} son, but the likelihood that George Foreman's two sons would have been prescribed the same exact Medications for these and any other Problems they have is minimal.

In a Doctors, Locations, and Procedures technique, information from a document complying with the CCDA can be used for identifying a unique patient. For example, each CCD, or document complying with the CCDA, is required to have specific information in the Header of the document denoting the Care Provider, Date, and Location. The body of the document contains Procedures and relative Dates. The high accuracy enabled when comparing patients' Doctors, Locations, and Procedures is a product of the inability for a Doctor to see more than one patient at the exact same time, the unlikelihood of that even if the doctor saw more than one patient at the same time, and at the same location, the Doctor still would have little ability to perform the same procedure at the same time on more than one patient.

In a CCDS data technique, additional Data from the CCDS, when available, offers increased accuracy in patient identification and matching. That is, comparing patient information including at least Patient Name, Sex, Date of birth, Race, Ethnicity, Preferred language, Smoking status, Medical Problems, Medications being taken, Medication allergies, Laboratory test(s) having been performed on the patient, values of the Laboratory result(s), Vital signs, Procedures, Care team member(s), Immunizations, Unique device identifier(s) for a patient's implantable device(s), Assessment and plan of treatment, Treatment Goals, Health concerns and the like, among different patients, greatly increases the accuracy of unique patient identification.

In some embodiments of a Unique Patient Identification method of a Data Command Center in accordance with the present principles, a Unique Patient Identification algorithm collects every available Identification Point, validates the points for presence of data, and assigns each Identification point a level of accuracy as it pertains to Patient Matching. Presence of data points with High Accuracy are prioritized and validated. Each Exact match is scored for accuracy. Each Likely Match is appropriately scored for accuracy. Each data point with no matching counterpart is negatively scored. Presence of data points with Moderate Accuracy are then prioritized and validated. Each Exact match is scored for accuracy. Each Likely Match is appropriately scored for accuracy. Each data point with no matching counterpart is negatively scored. Moderate accuracy data points are scored lower than High accuracy data points. Presence of data points with Low Accuracy are then prioritized and validated. Each Exact match is scored for accuracy. Each Likely Match is appropriately scored for accuracy. Each data point with no matching counterpart is negatively scored. Low accuracy data points are score lower than Moderate accuracy data points.

Upon gathering and analyzing all available data for Unique Patient Identification, scores are tallied and compared to an acceptable Matching Threshold. In some embodiments of the present principles, the Matching Threshold is configured to clearly exceed a matching accuracy of current patient identification techniques with the inclusion of far more points of identification to compare. In some embodiments, the matching of the present principles can occur without the requirement of matching on current PII data. For example, George Edward Foreman IV may have been staying with a friend in Florida when he visited a doctor. Not wanting to be identified as the son of the famous boxer, he purposely listed his name as G. Foreman and address as the place he was staying. Date of birth may have been left blank. A positive identification can still be made, in accordance with the present principles, if the clinical data supplied matches with a high enough degree of accuracy clinical data stored for George Edward Foreman IV, such as the unique identifier on his knee replacement or the fact that a large number of Doctors, Locations, Procedures, Problems, Allergies, Medications, and Lab Results are found to be matching, while the name, address, and date of birth have non-matching counterparts.

A Unique Patient Identification algorithm of the present principles can reach a logical end when a positive match is determined, or no positive match can be made. In some embodiments, should no positive match be made, the patient and possible matches can be flagged for human intervention.

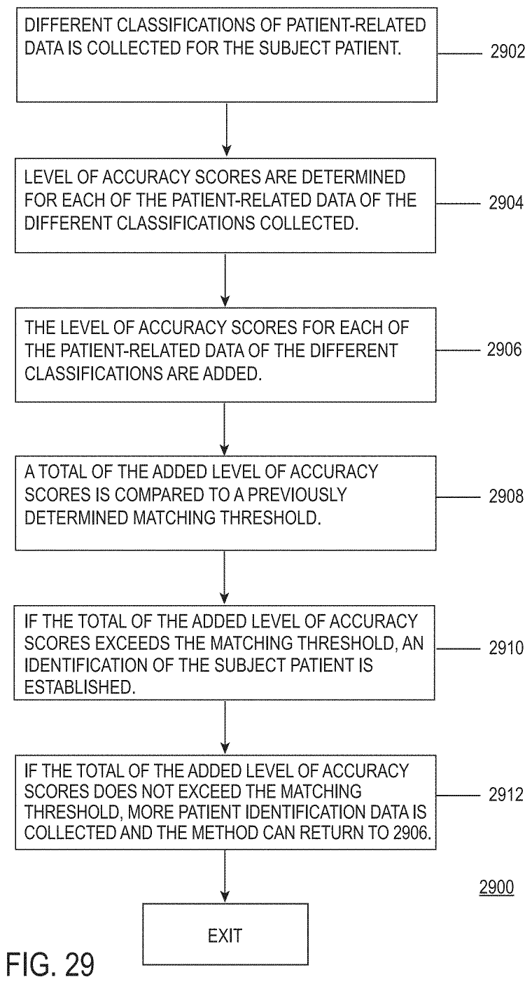

FIG. 29 depicts a flow diagram of a method for Unique Patient Identification for a subject patient in a Data Command Center including patient-related data received or derived from at least one patient database in accordance with an embodiment of the present principles. The method 2900 of FIG. 29 illustratively begins at 2902 during which different classifications of patient-related data is collected for the subject patient. For example and as described above, in some embodiments, data from the Common Clinical Data Set and other sources can be collected to be used in patient identification techniques of the present principles. The method 2900 can proceed to 2904.

At 2904, level of accuracy scores are given for each of the patient-related data of the different classifications collected. The method 2900 can proceed to 2906.

At 2906, the level of accuracy scores for each of the patient-related data of the different classifications are added. The method 2900 can proceed to 2908.

At 2908, a total of the added level of accuracy scores is compared to a previously determined matching threshold. The method 2900 can proceed to 2910.

At 2910, if the total of the added level of accuracy scores exceeds the matching threshold, an identification of the subject patient is established. The method 2900 can proceed to 2912.

At 2912, if the total of the added level of accuracy scores does not exceed the matching threshold, more patient identification data is collected and the method 2900 can return to 2906. The method 2900 can then be exited.

It is critical for a medical care provider to know what medications a patient has ever taken or is currently taking, what the frequency is, why the medication was taken or discontinued and reasons for switching to another medication. There is currently no medication management tool that visually correlates the clinical parameters or disease state findings that the medication is prescribed to have an impact on. A Data Command Center of the present principles via at least one of a medical records dashboard and a Medications Management chart or tool in accordance with the present principles enables a user to correlate frequency, amount and types of medications taken to enable the user to visualize how that medication affects the parameters reviewing modulation such as blood pressure, eye pressure, weight, heart rate, etc. and corresponding it to when the medications were taken to see if there is a cause and effect. There is no system that can also correlate and display on a view surgical intervention, an injection or any other intervention and see how these additional factors correlate with timing of medication taken and how all this impacts clinical finding, measurements, disease progression and symptoms. A Data Command Center of the present principles enables a user to visually correlate diagnostic tests and images that may show how all these treatment modalities result in changes or lack thereof on lab results, imaging, etc. For example and as enabled by embodiments of the present principles, if a patient is being treated for cancer and chemotherapeutic medication can be seen with direct access on one screen with x-rays taken overtime showing changes in size of a tumor or mass along with the labs or clinical symptom changes all in context of when surgical or radiation therapy intervention was performed, enables medical care providers to efficiently and accurately make medical decisions.

Embodiments of a Data Command Center of the present principles can also be linked to a Pharmaceutical system or other provider of prescribed medication (i.e., E-prescribe or a similar system) such that a medical care provider is enabled to accurately track when medication was actually received by a patient. It can be very difficult if not impossible with current systems for a medical care provider to know when a medication was actually received by a patient. That is, medical care providers often rely on scribes to write prescriptions and when patients call to refill the medication, often it is not the medical care provider who prescribes the refills of medication but an assistant who does so. Even further, just because a medical care provider orders a drug fora patient that does not mean the patient actually went and got it filled or that the medication was taken as prescribed. To further complicate matter, patients can be given different medication than prescribed by the medical care provider because a generic drug instead of a brand drug could have been given.

Embodiments of a Data Command Center of the present principles can also be linked to home monitoring devices or system for being able to more accurately determine when medication was actually taken by a patient. That is, just because medications are prescribed and received by a patient does not mean that the patient has started taking the medication or even taking it as prescribed. A patient may also misunderstand what the doctor actually wants the patient to do and is actually taking the medication incorrectly. Embodiments of a Data Command Center via, for example, a medical records dashboard of the present principles enable medical care providers to more accurately track medications and how they are being taken by patients, which improves quality of care. More specifically, in accordance with the present principles, a medical care provider is enabled to visualize the medications, the start and stop dates, reasons for discontinuation, and is enabled to manage and change the display based on reality they confirm with the patient at point of care and via the pharmaceutical and home monitoring devices that can be linked into the Data Command Center of the present principles.

As described above, embodiments of a Data Command Center via, for example, at least one of a medical records dashboard and a Medication Management chart/tool of the present principles enables medical care providers to more accurately track medications and dates associated with the medications, for example in rows and columns. In some embodiments a Data Command Center via, for example, at least one of a medical records dashboard and a Medication Management chart/tool of the present principles can display tracked medication information in graph form. In some embodiments, each medication or class of medications associated with a patient can be represented by a bar graph or a linear graph or other visual method or means that in either the vertical direction or in a horizontal direction the doctor can visualize the actual start and stop dates of all relevant medications for a patient, which can all be seen simultaneously with any other relevant data that the medications can impact. More specifically, in some embodiments, a Data Command Center in accordance with the present principles, such as the Data Command center 001 of FIG. 1, can further include the ability to intelligently display medications in context (referred to by the inventors in some embodiments as Medication Management), by grouping, categorizing, expanding, contracting, displaying, hiding, and highlighting or flagging medications to visually present medications to a user of the Data Command Center (e.g., medical care provider) in a medical records dashboard in a manner that makes such medication more easily identifiable by the user. In one embodiment, Medication Management exists as a series of intelligent vertical columns representing individual medications, classes of medications, categories of medications, or logical groupings of medications, differentiating medications by color or combinations of colors, symbols, and/or text, graphing start and stop dates and times or individual doses correlated to relevant values and relevant events. In accordance with the present principles, graphical differentiation between medications can consist of individual colors for individual medications, combinations of colors for medications including more than one component, or complex graphical representations. In some embodiments, color standards, such as defined by the American Academy of Ophthalmology, can be used for color coding the medications and/or custom colors can be used. For example, in ophthalmology and with respect to eye care, medications have been assigned in the industry to have a certain color on the eye drop bottle or cap. In some embodiments, these colors can be displayed allowing recognition by the user of the class of medication. For instance, yellow is a beta blocker one of which is Timoptic. In accordance with the present principles, medical care providers who have memorized the color caps can instantly recognize, by viewing a medical records dashboard of the present principles, the class of medication without even seeing the name.

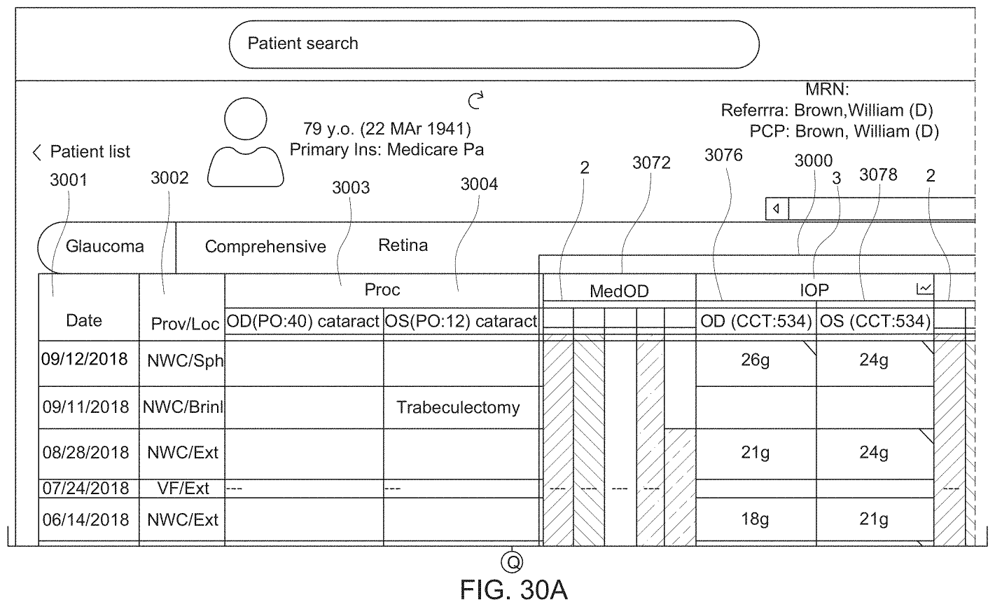

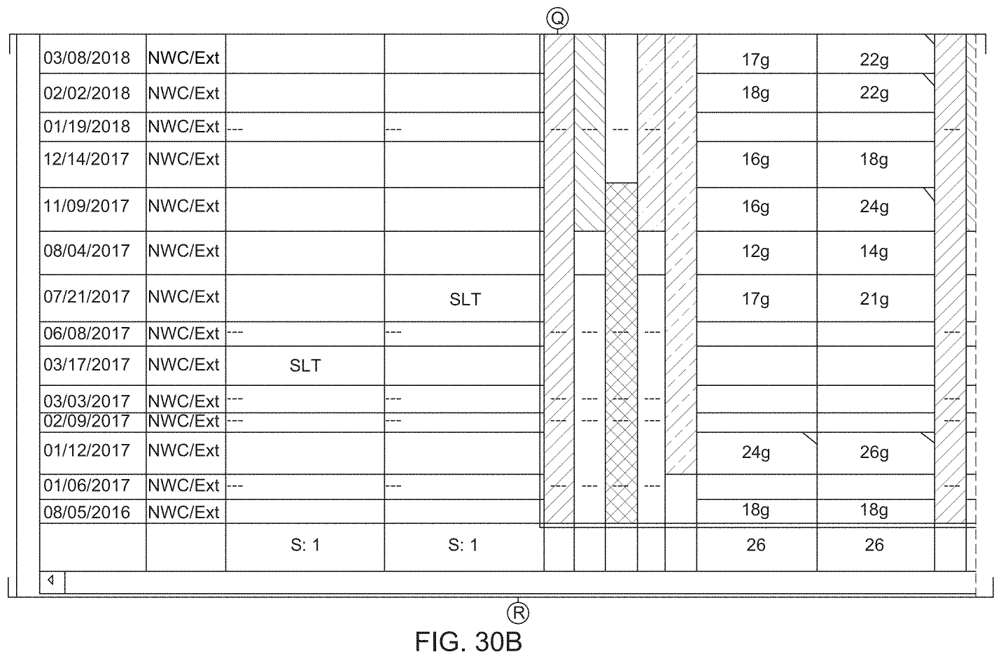

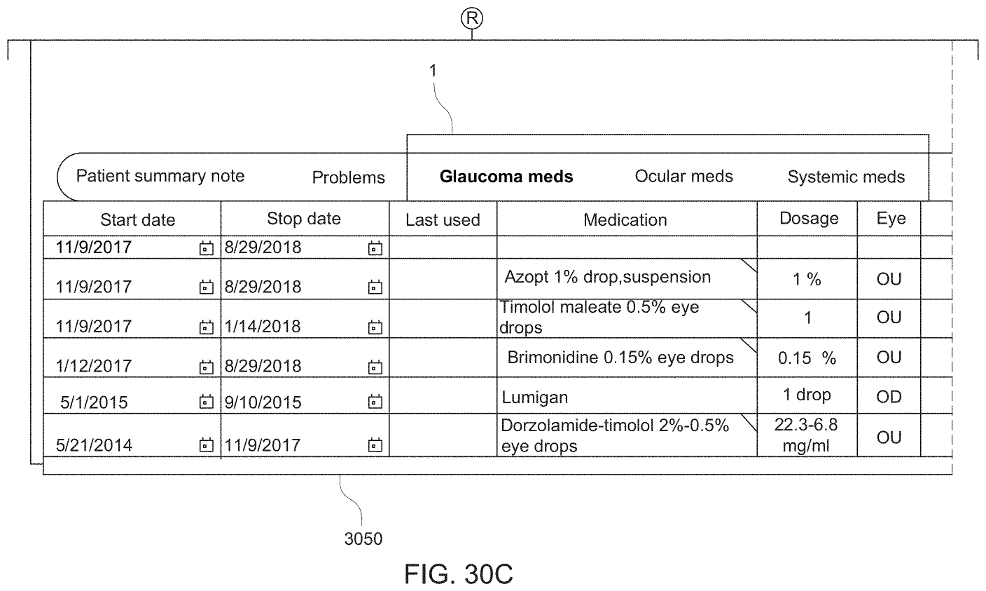

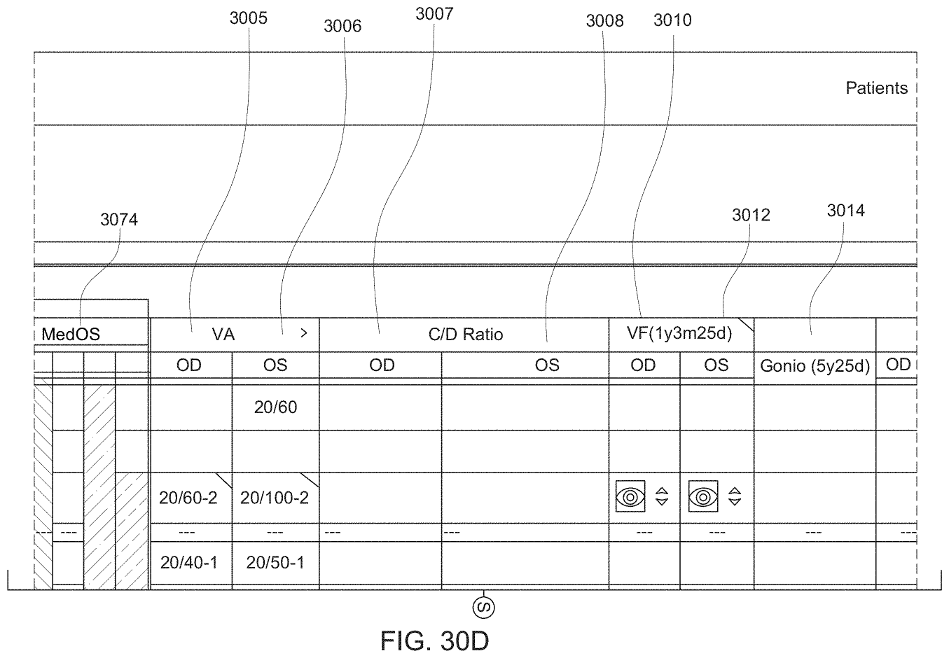

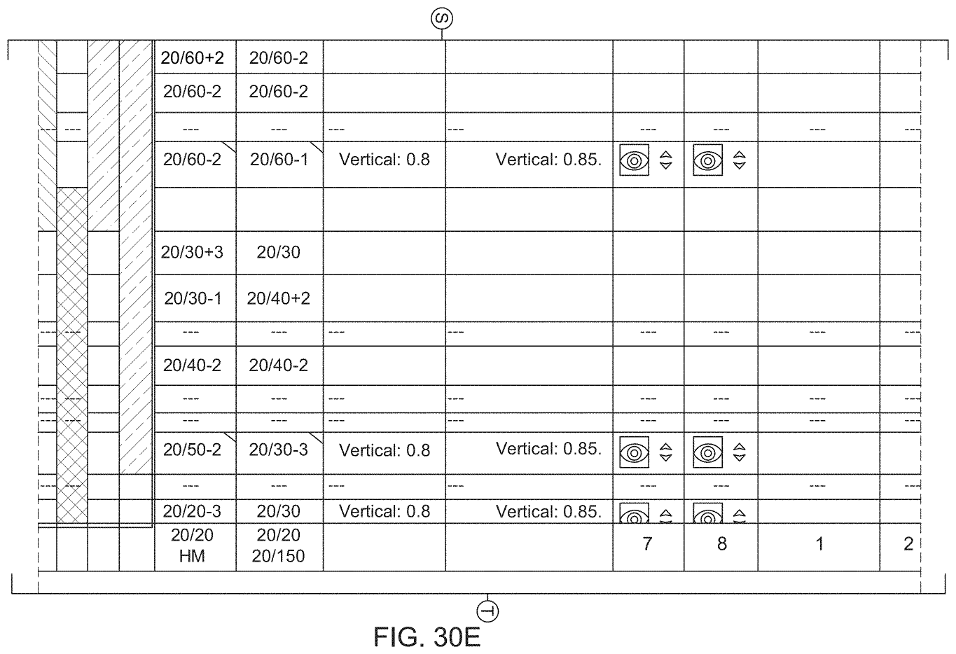

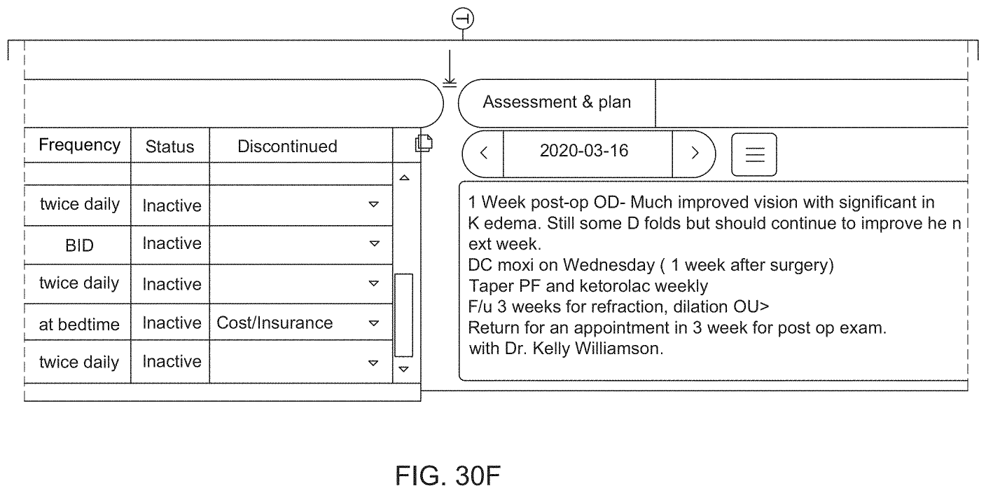

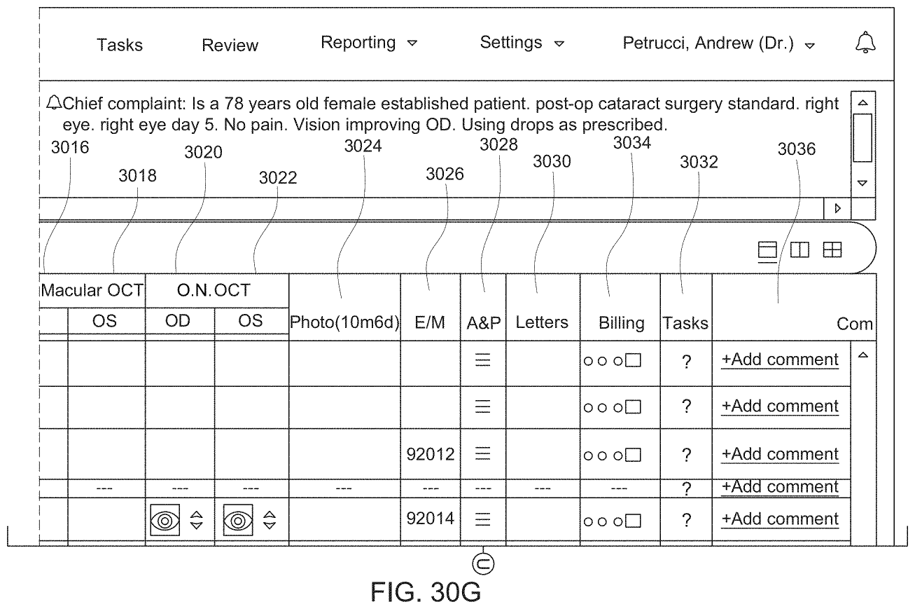

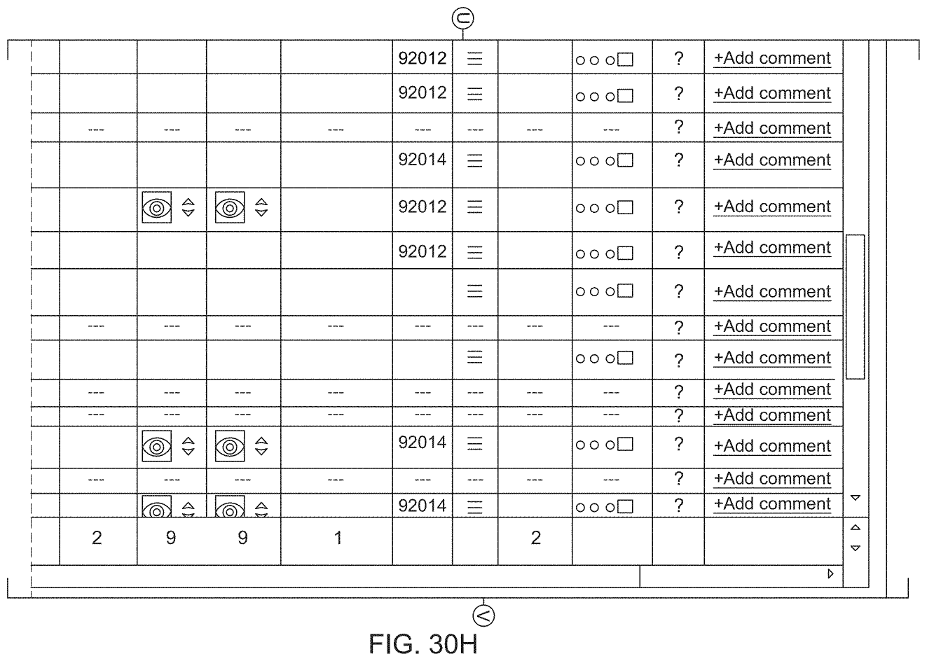

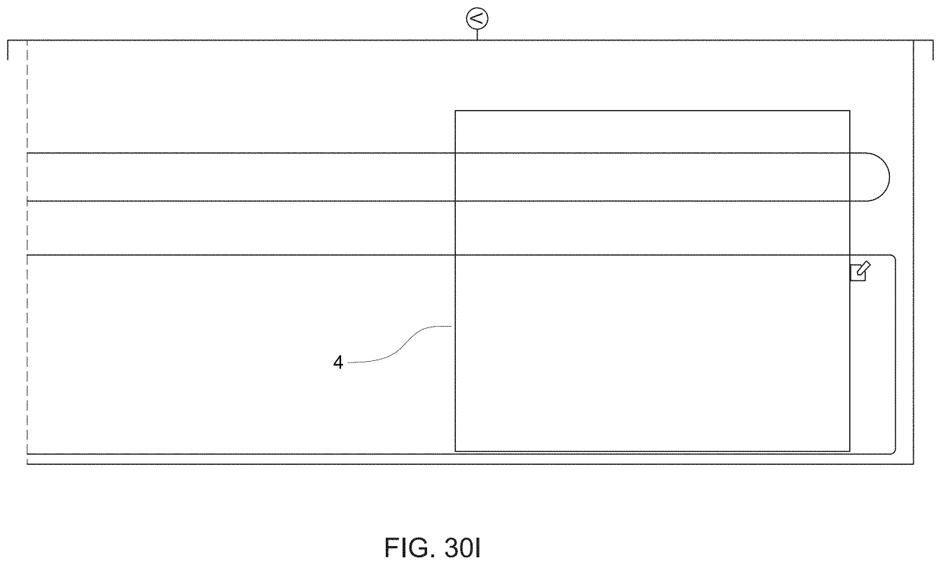

For example, FIGS. 30A, 30B, 30C, 30D, 30E, 30F, 30G, 30H, and 30I a first embodiment of a Medication Management chart 3000 that can be displayed in at least a portion of a medical records dashboard of the present principles in accordance with one embodiment. The medical records dashboard of FIGS. 30A, 30B, 30C, 30D, 30E, 30F, 30G, 30H, and 30I illustratively comprises a patients Glaucoma chart including a date column 3001, a Provider/Location column 3000, a Procedures column for a right eye 3003 and for a left eye 3004, the Medications Management Chart 3000, a VA column for the right eye 3005 and for the left eye 3006, a C/D Ratio column for the right eye 3007 and for the left eye 3008, a VF column for the right eye 3010 and for the left eye 3012 including a Gonio column 3014, a Macular OCT column for the right eye 3016 and for the left eye 3018, an O.N. OCT column for the right eye 3020 and for the left eye 3022, a Photo column 3024, an E/M column 3026, an A&P column 3028, a Letters column 3030, a Tasks column 3032, a Billing column 3034, and a Comments column 3036 all arranged to depict information in rows of the medical records dashboard of FIG. 30 by date.

The Medications Management Chart 3000 of FIGS. 30A, 30B, 30C, 30D, 30E, 30F, 30G, 30H, and 30I includes a Medication column for the right eye 3072 and the left eye 3074, illustratively on either side of an IOP column for a right eye 3076 and the left eye 3078. all arranged to depict information in rows of the medical records dashboard by date. In the Medications Management Chart 300 of FIGS. 30A, 30B, 30C, 30D, 30E, 30F, 30G, 30H, and 30I, the Medication column for the right eye 3072 and the left eye 3074 are illustratively separated into sections for separately displaying bars for each of a plurality of available medications. The embodiment of FIGS. 30A, 30B, 30C, 30D, 30E, 30F, 30G, 30H, and 30I depicts an example of a medical records dashboard including medication management in the field of eye care, however embodiments of the present principles can be applied to substantially any medical specialty and the like.

In the embodiment of FIGS. 30A, 30B, 30C, 30D, 30E, 30F, 30G, 30H, and 30I, the pressure of each eye of a patient is measured from 0 to 50. In addition, each of the medications taken associated with each respective eye of the patient are depicted in bar graph form and distinguished by color according to the dates taken. In the embodiment of the medical records dashboard of FIGS. 30A, 30B, 30C, 30D, 30E, 30F, 30G, 30H, and 30I, the color bars representing the medications administered to the patient are displayed adjacent to respective pressure data points for each eye according to a date administered to allow the user to directly correlate the effect of the medication on a respective eye. In the embodiment of FIGS. 30A, 30B, 30C, 30D, 30E, 30F, 30G, 30H, and 30I, section #2 depicts the medication bar graphs, section #3 depicts clinical measurements of eye pressures that are affected by the medications, and window #4 depicts an ordering panel enabling the ordering of medication through, for example, E-prescribe, DoctorFirst, or other methods. In the embodiment of FIGS. 30A, 30B, 30C, 30D, 30E, 30F, 30G, 30H, and 30I, window #1 depicts an embodiment and location of a control panel 3050 of the medical records dashboard, which identifies which medications are represented by which colors and identified a dosage, a frequency and a status of the medications being administered to a patient.

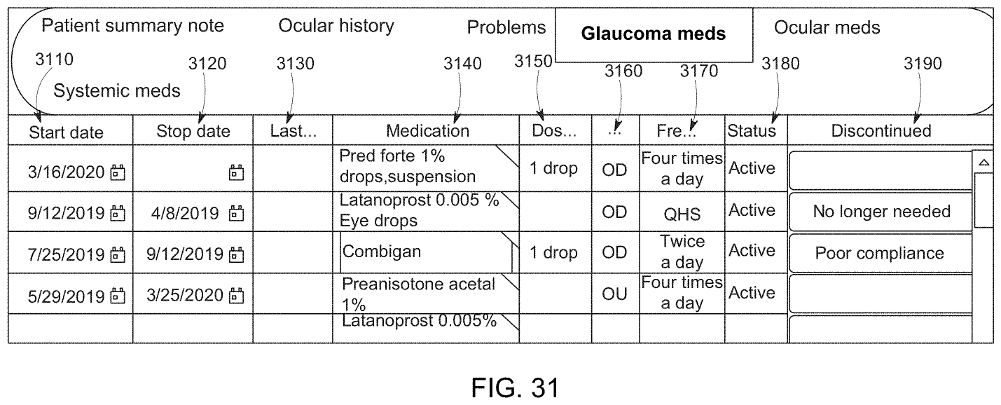

For example, FIG. 31 depicts an embodiment of the control panel #1 of the Medication Management chart of FIGS. 30A, 30B, 30C, 30D, 30E, 30F, 30G, 30H, and 30I in accordance with an embodiment of the present principles. The control panel of FIG. 31 illustratively includes a Start Date Column 3110 depicting a start date of a medication in a respective row, a Stop Date Column 3120 depicting a stop date (if any) of the medication in the respective row, a Last Column 3130 depicting a date when the medication in the respective row was last taken, a Medications Column 3140 depicting medications taken by the patient, a Dosage Column 3150 depicting a dosage amount of the medication taken by the patient, a Location Column 3160 indicating in what part of the patient's body the medication was applied, a Frequency Column 3170 indicating how often the medication is being applied, a Status Column 3180 depicting if the medications are or are not currently being applied, and a Discontinued Column 3190 depicting a reason for discontinuance of the medication (if a reasons exists). As depicted in FIG. 31, in accordance with some embodiments of the present principles, the Medications Column 3140 can be color coded such that each medication comprises a respective color.

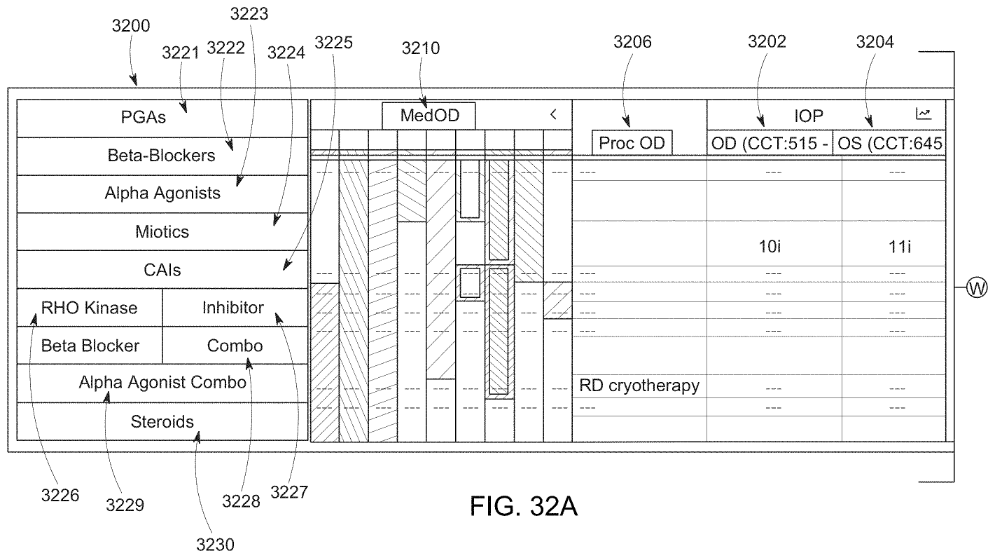

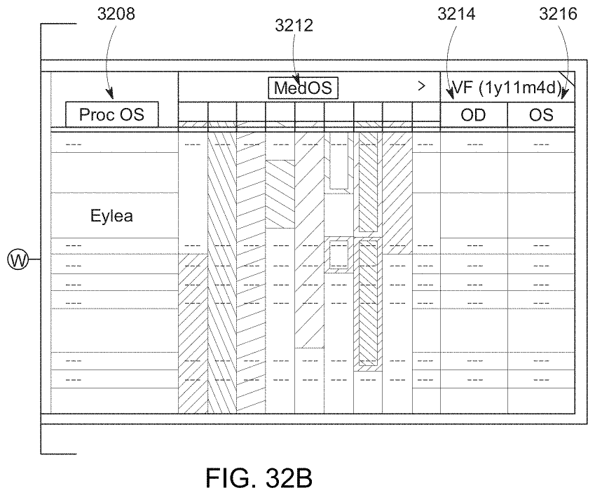

For example, FIGS. 32A and 32B depict a Medication Management Chart 3200 that can be displayed as part of a medical records dashboard or as a stand-alone Medication Management tool in accordance with an embodiment of the present principles. In the embodiment of FIGS. 32A and 32B, the Medication Management Chart 3200 includes a center section including respective columns depicting an intraocular pressure (IOP) for a patient's right eye 3202 and an intraocular pressure (IOP) for the patient's left eye 3204 on various different dates. In FIGS. 32A and 32B, next to the respective pressure columns for the patient's right eye 3202 and the patient's left eye are respective columns depicting respective procedures performed on the patient's right eye 3206 and the patient's left eye 3208 on the different dates. The Medication Management Chart 3200 of FIGS. 32A and 32B further includes respective columns depicting respective medications administered to the patient right eye 3210 and the patient's left eye 3212 on the different dates. The Medication Management Chart 3200 of FIGS. 32A and 32B further includes a visual field (VF) column for the right eye 3214 and a VF column for the left eye 3216.

The Medication Management Chart 3200 of FIGS. 32A and 32B further illustratively includes a color-coded key identifying medications present in the Medication Management Chart 3200. In the embodiment of FIGS. 32A and 32B, medications administered to the patient's eyes include PGAs 3221, Beta-Blockers 3222, Alpha Agonists 3223, Miotics 3224, CAIs 3225, Rho Kinase 3226 and Inhibitor 3227, Beha-Blocker Combo 3228, Alpha Agonist Combo 3229, and Steroids 3230. As depicted in the Medication Management Chart 3200 of FIGS. 32A and 32B, in some embodiments, combinations of drugs can exist and can be depicted as a combination of the colors of the drugs that make-up the drug combination. Although in the embodiment of FIGS. 32A and 32B, the Medication Management Chart 3200 illustratively comprises a color-coded key for identifying the medications, in other embodiments of a Medication Management Chart of the present principles, a color-coded key does not have to be included. In addition, although in the embodiment of the present principles depicted in FIGS. 32A and 32B, the Medication Management Chart 3200 depicts a combination of drugs as a bar having one color representing a first drug and a box around the bar in a second color representing a second drug of the combination, in some embodiments a drug combination can be represented using a cross-hatch method, in which stripes in a bar are a first color representing a first drug and the rest of the bar is as second color representing the second drug of the combination. In accordance with the present principles, a drug combination can include more than two drugs and drugs and drug combinations can be represented by assigning a color to each drug and if a medication has more than one drug in it then all colors can be displayed by any means in, on or around the bar representing that medication.

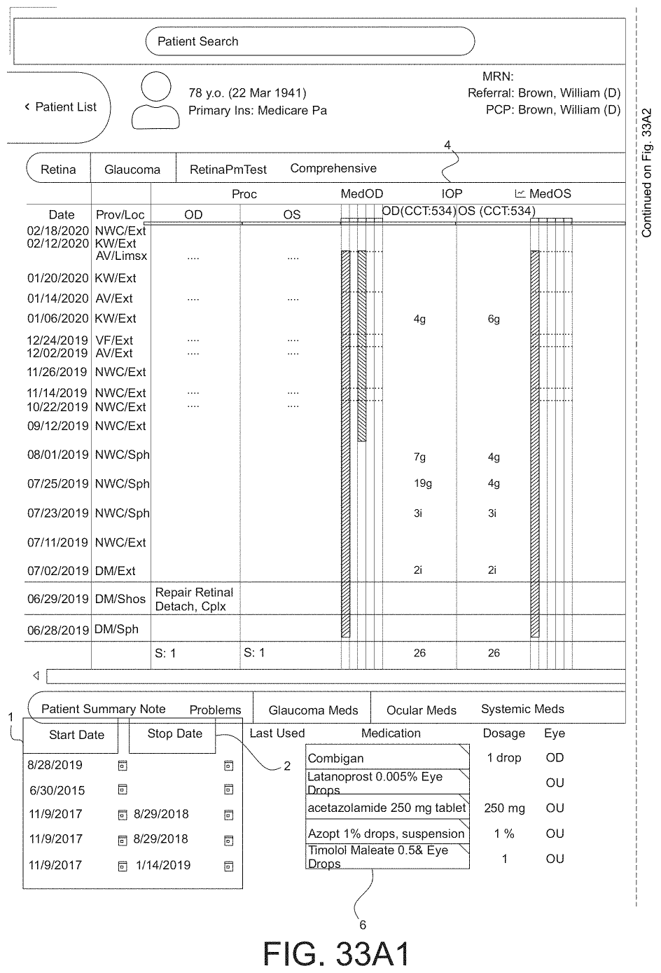

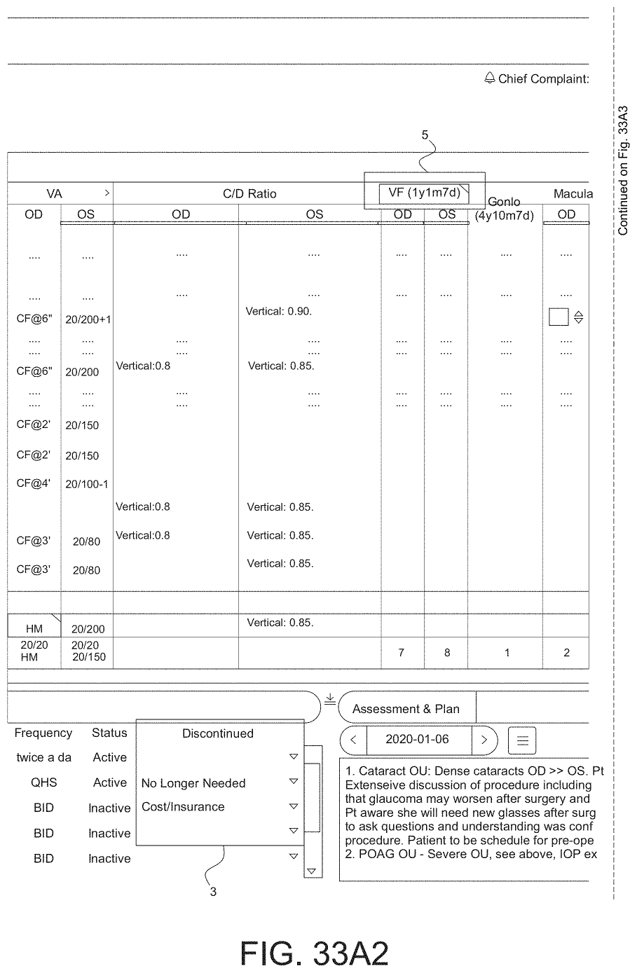

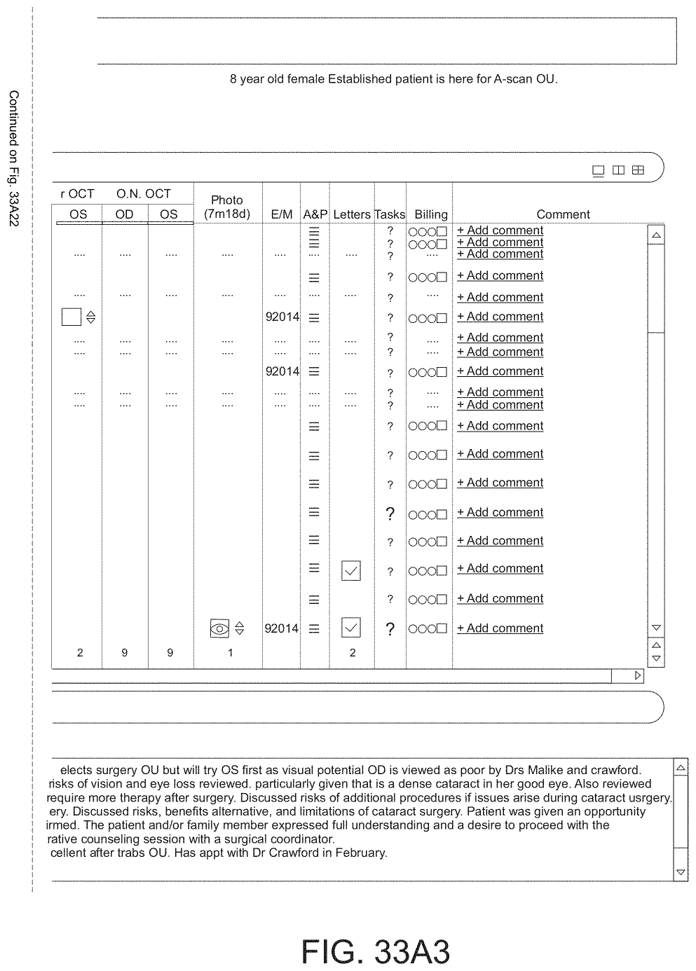

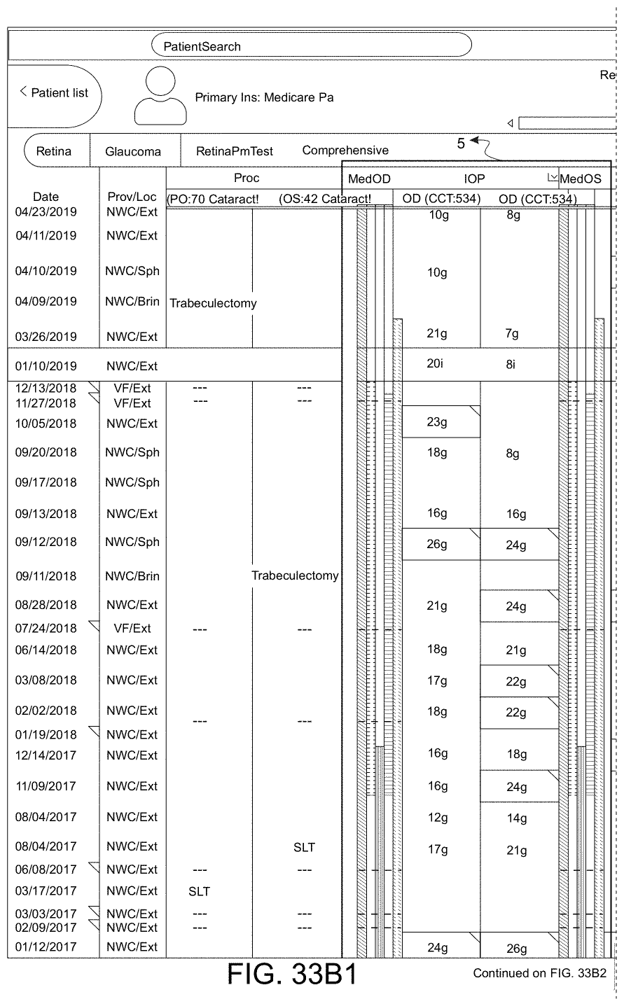

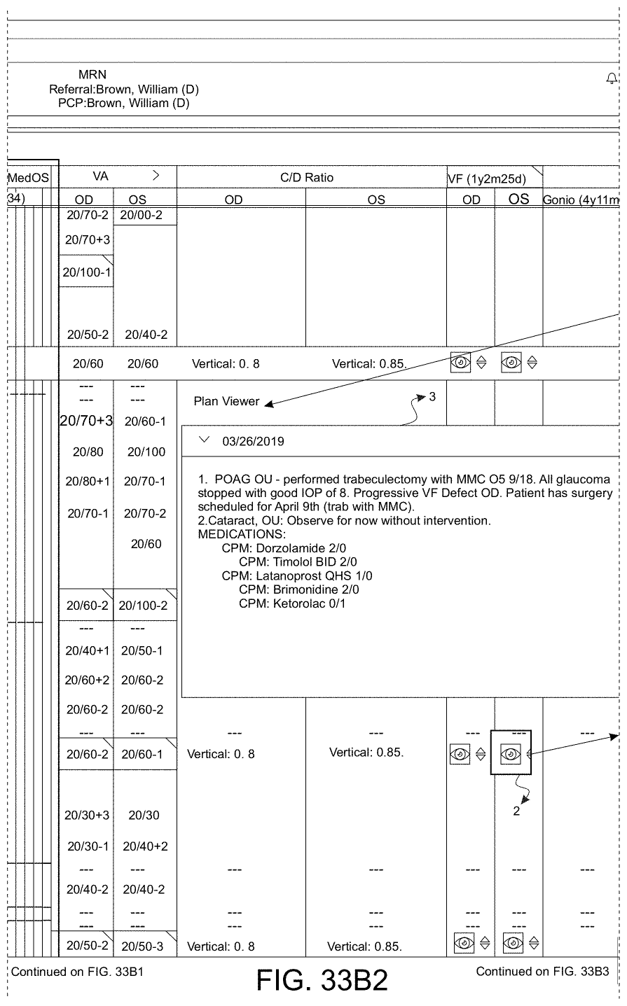

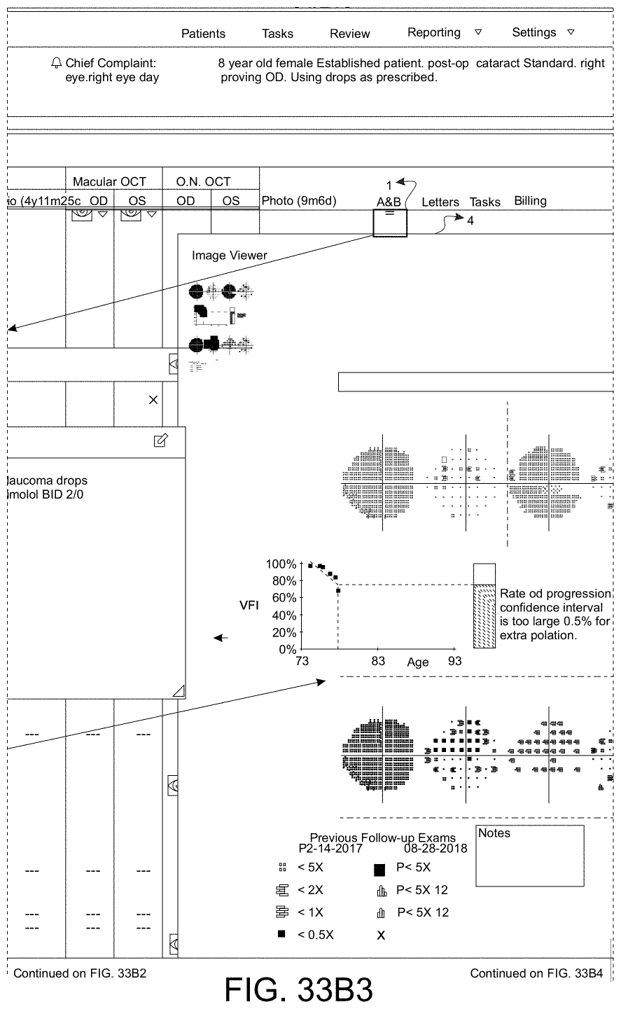

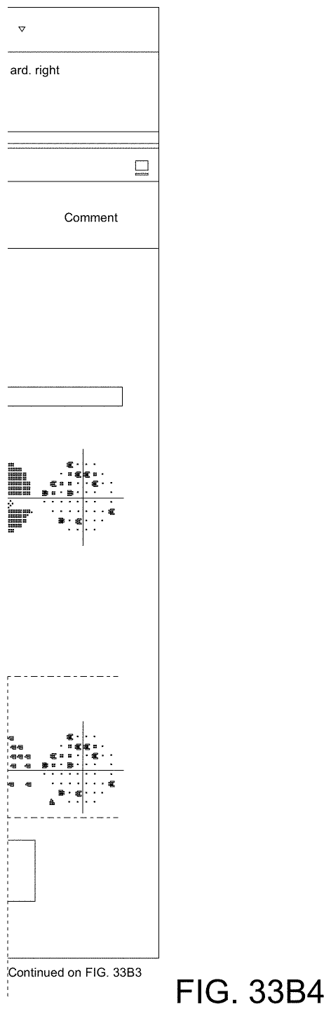

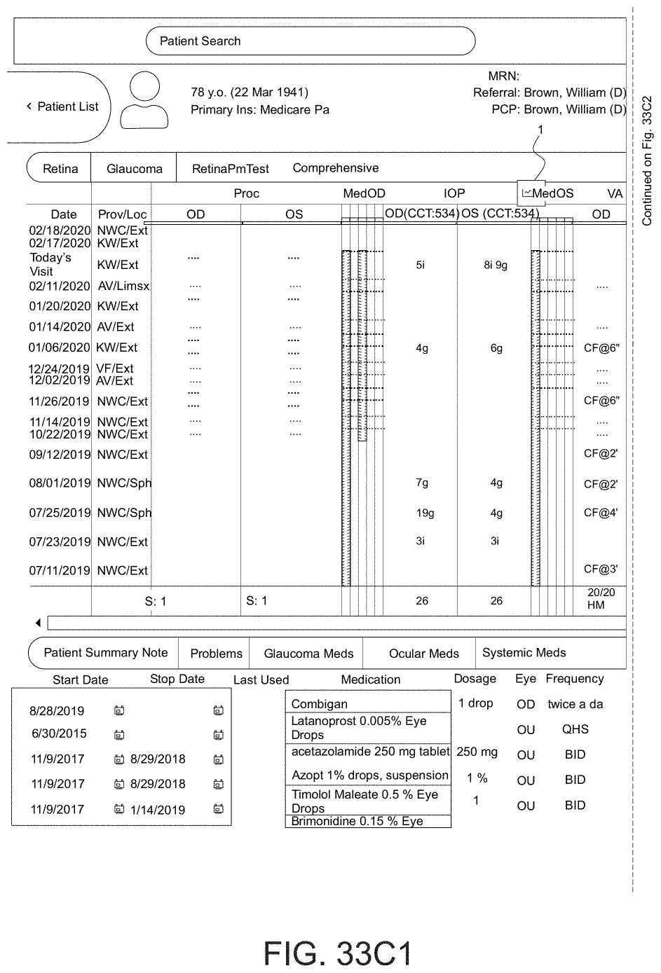

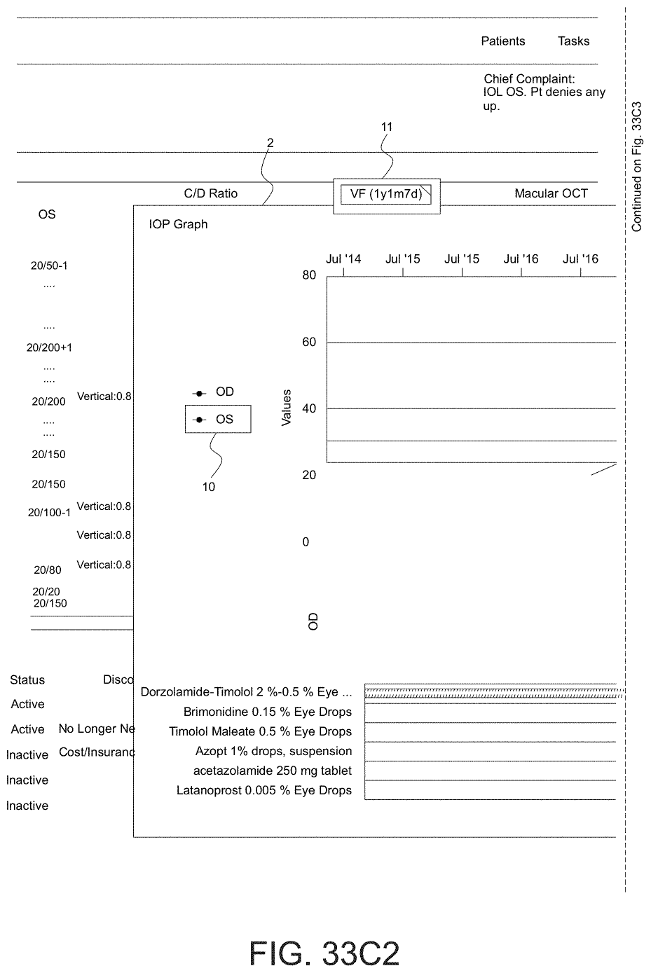

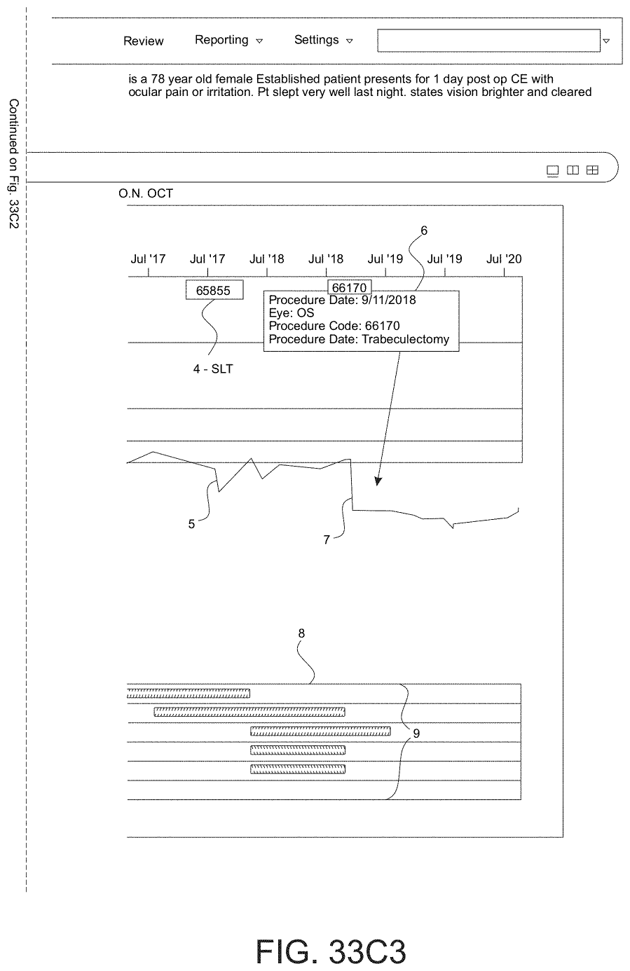

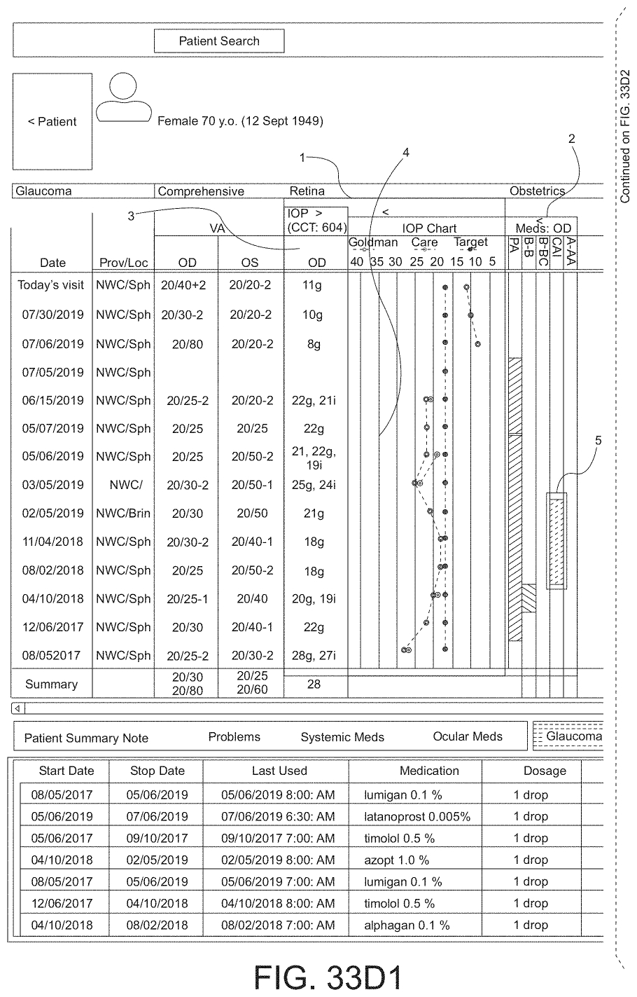

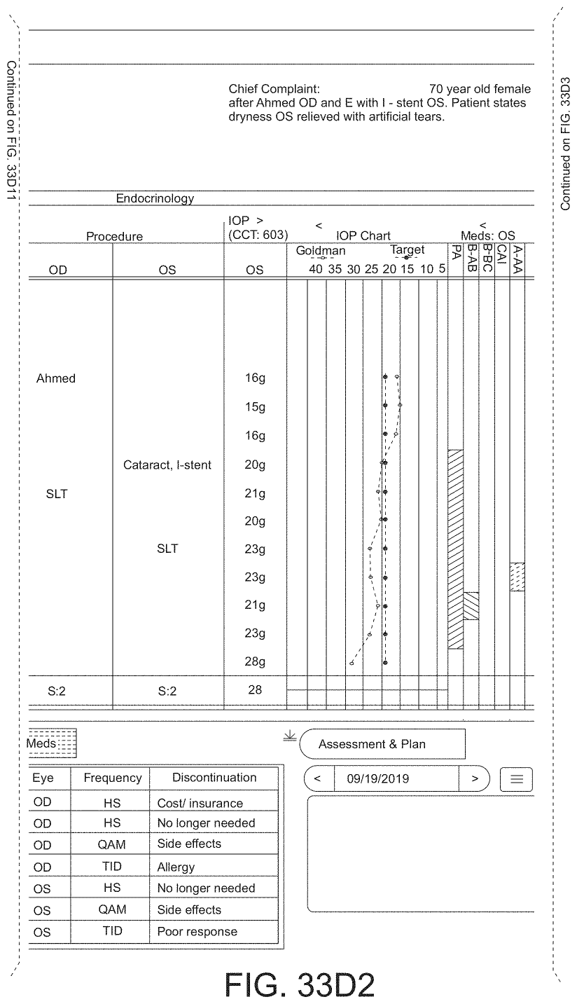

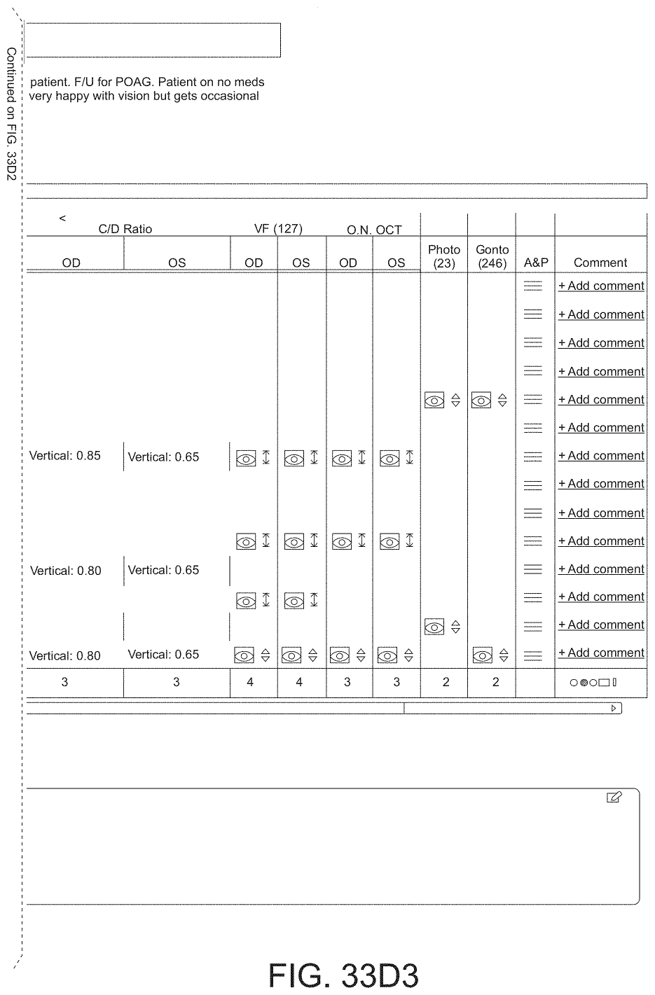

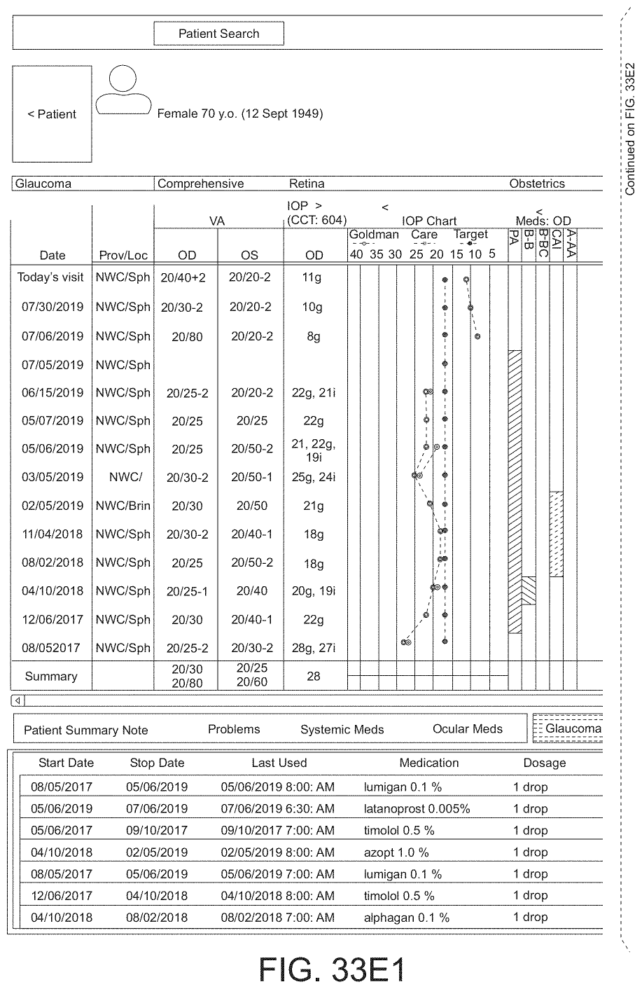

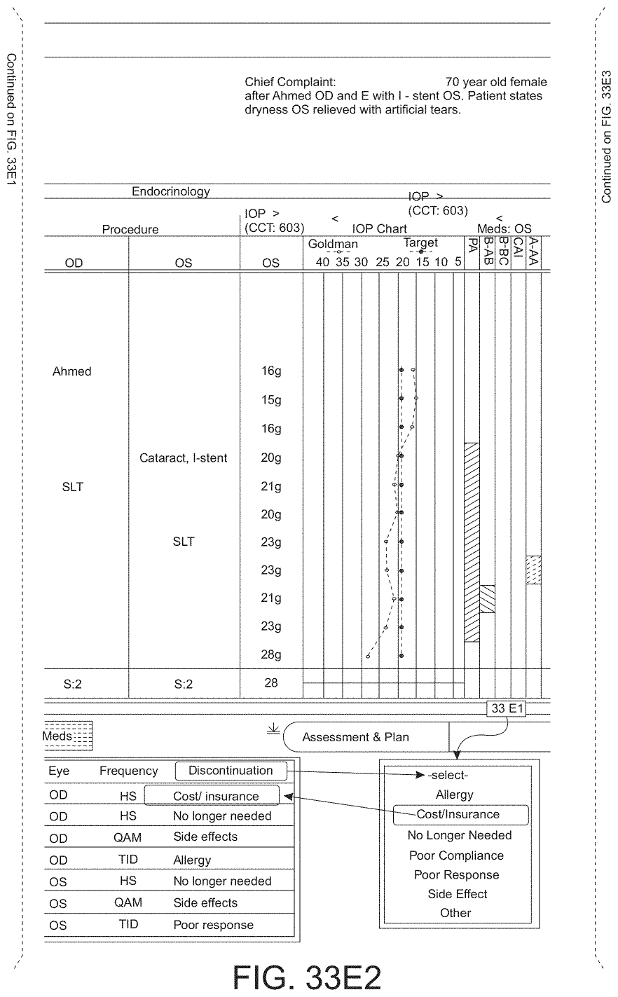

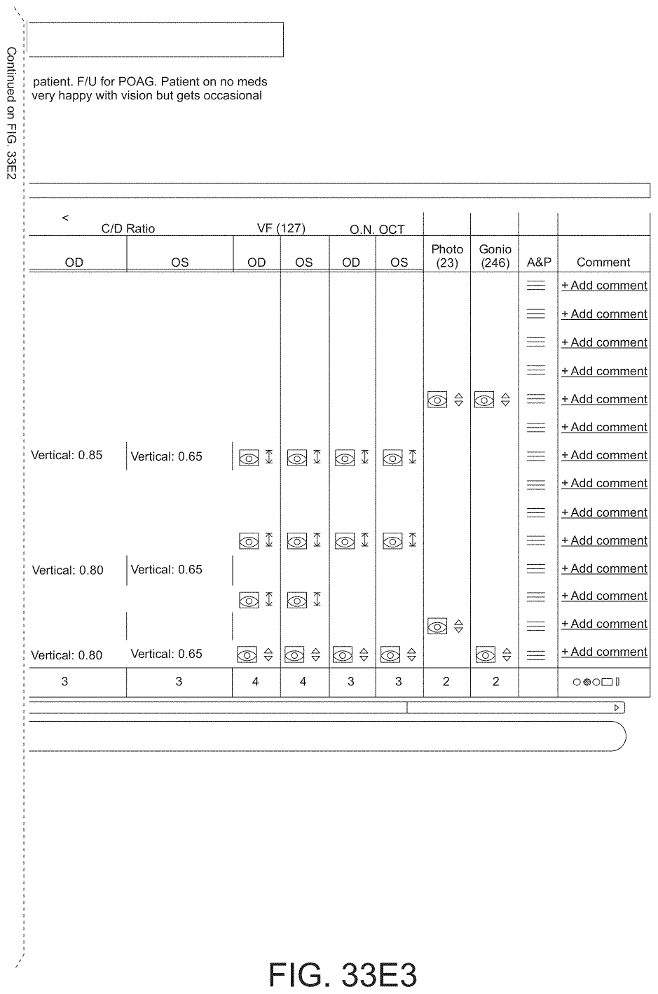

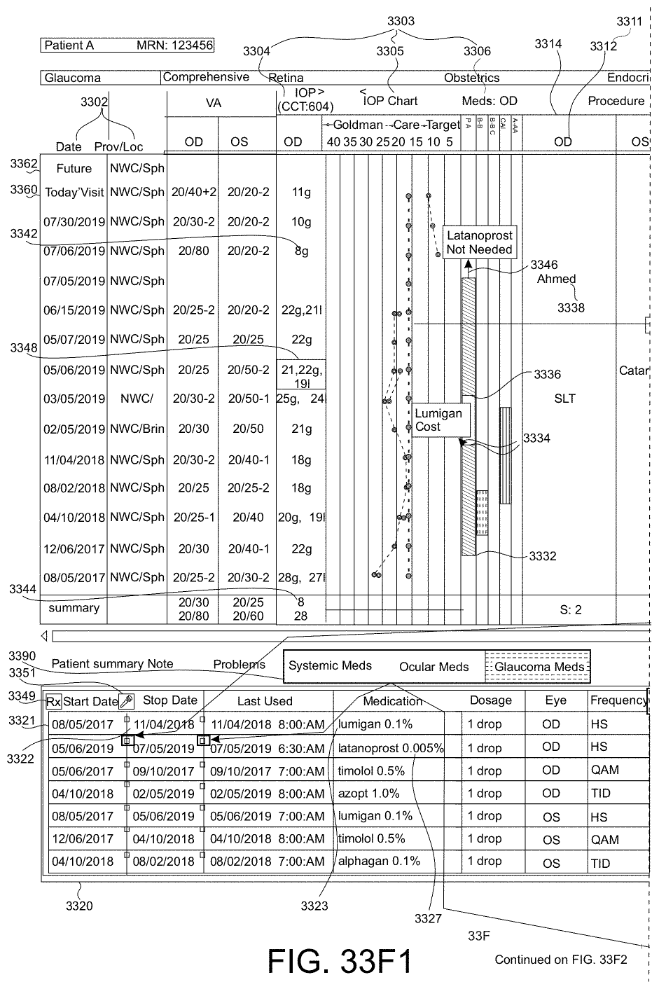

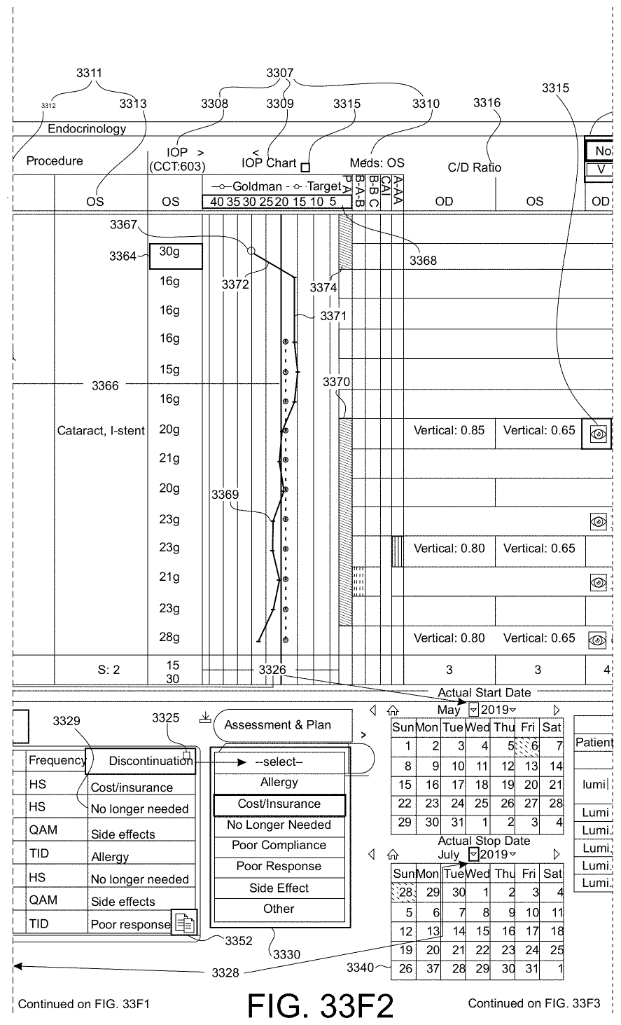

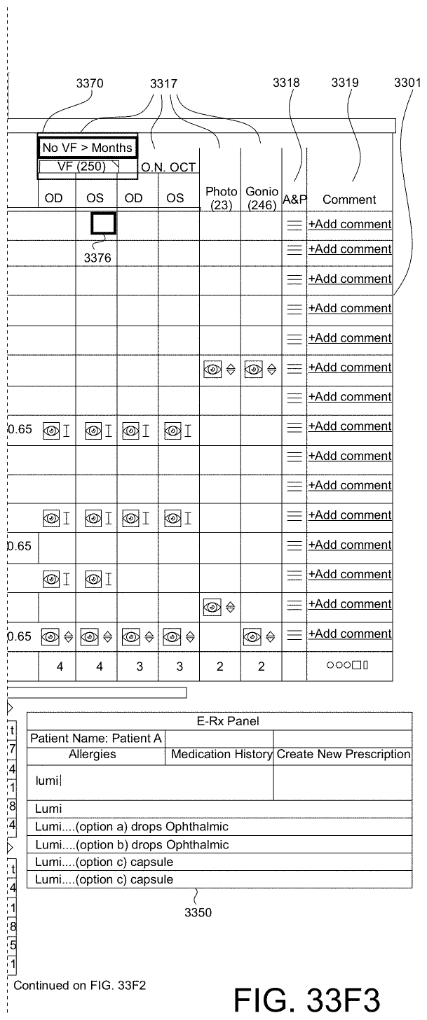

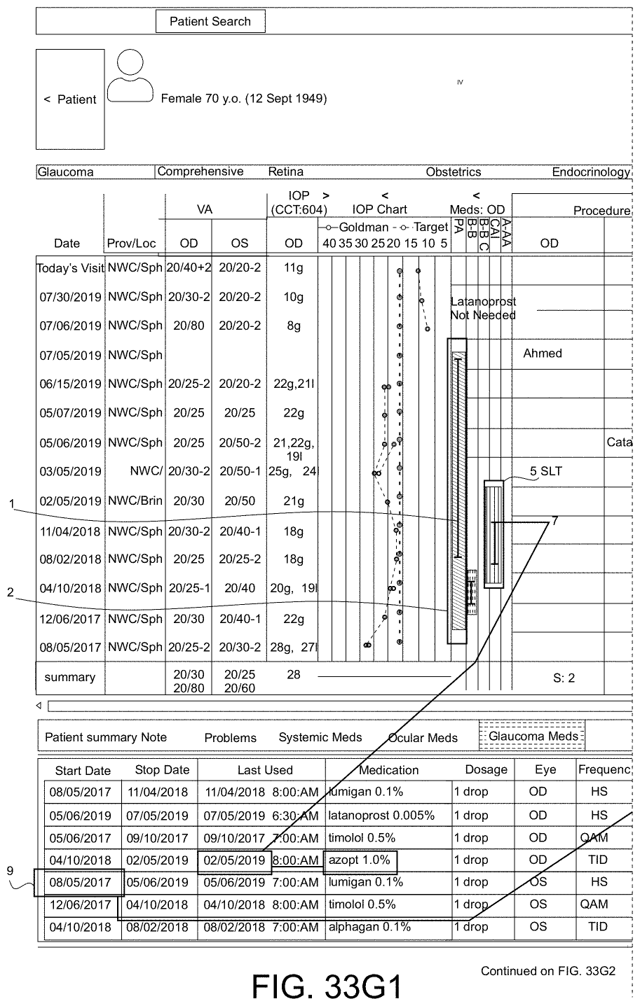

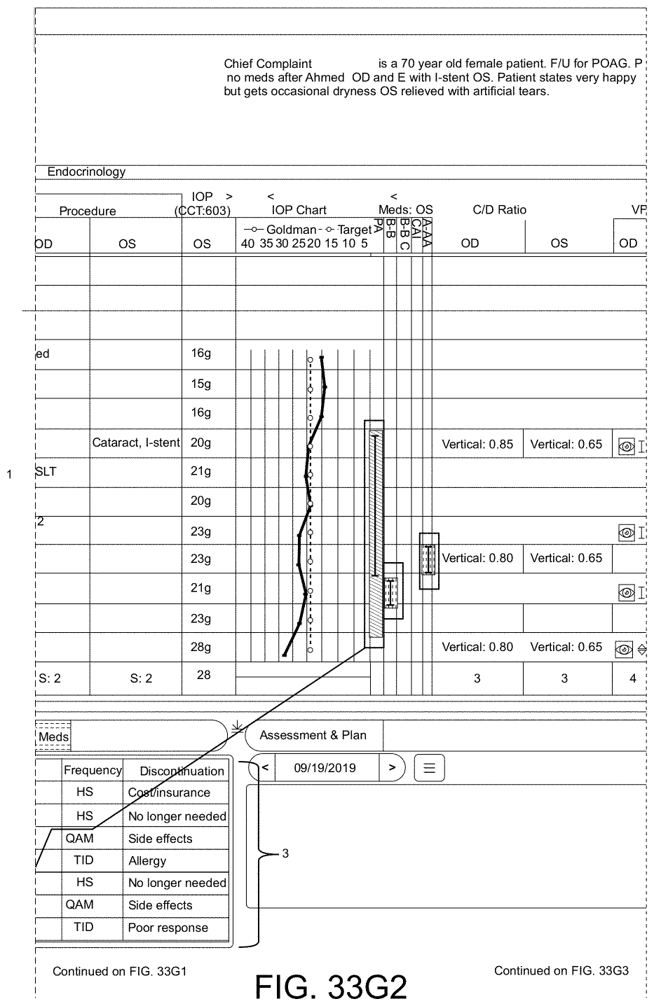

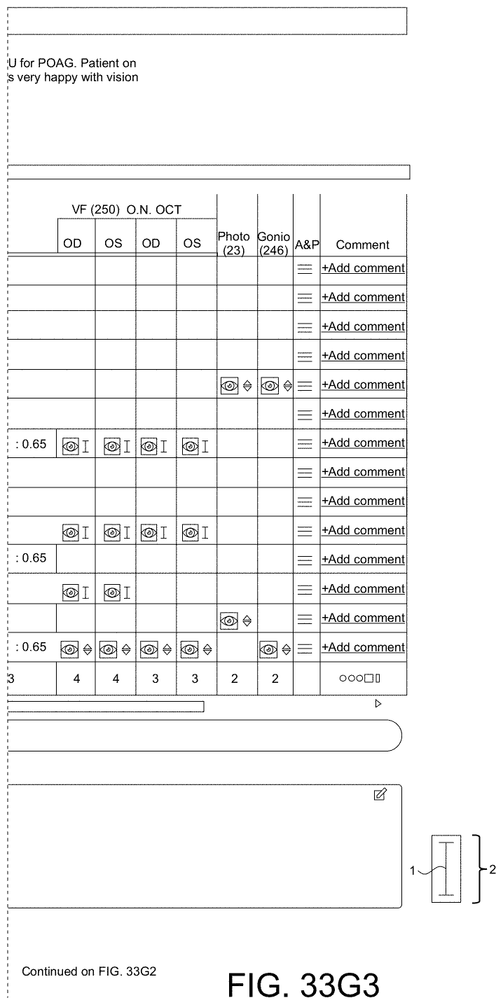

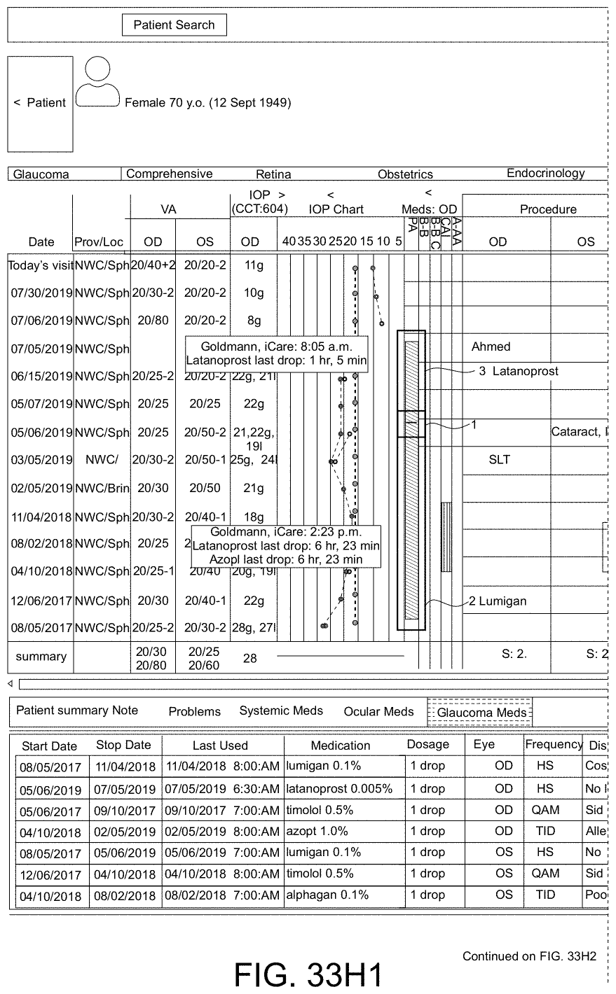

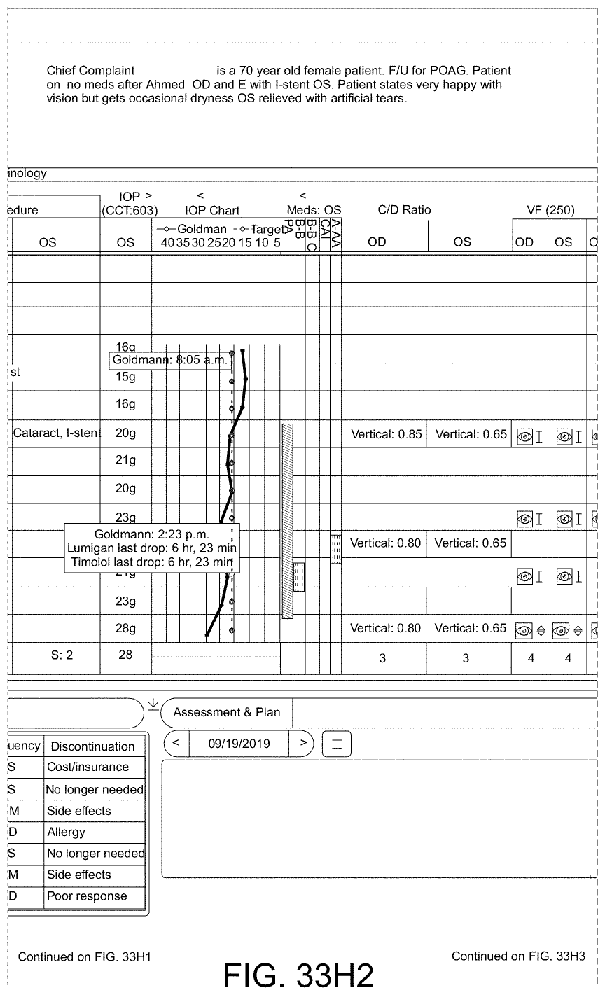

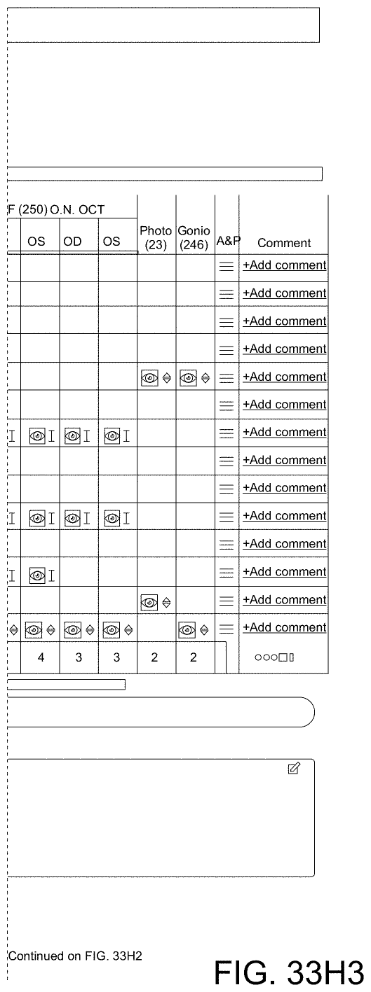

FIGS. 33A1-3, B1-4, C1-3, D1-3, E1-3, F1-3, G1-3, H1-3, and I1-3 depict embodiments of a Medication Management Chart having different features in accordance with the present principles and will be described with reference to the medical records dashboard and the Medications Management Chart 3000 of FIGS. 30A, 30B, 30C, 30D, 30E, 30F, 30G, 30H, and 30I. FIGS. 33A1, 33A2, and 33A3 depict an example of how the Control Panel #1 of FIGS. 30A, 30B, 30C, 30D, 30E, 30F, 30G, 30H, and 30I can be implemented by a user to identify start and stop dates for the various medications taken by a user in accordance with an embodiment of the present principles. FIGS. 33A1, 33A2, and 33A3 further depict how section 2 of the Control Panel #1 can be implemented to identify and assign colors for the medications and section 3 of the Control Panel #1 can be implemented to note reasons for a patient discontinuing a medication.

FIGS. 33B1-4 depict an embodiment of a Medication Management Chart in which icons can be activated to bring up additional information in accordance with an embodiment of the present principles. In FIGS. 33B1-4, element 1 depicts how by clicking on a column heading, information available under the column heading, such as a note inserted by a user, can be accessed, for example, in a pop-up window, element 3. In FIGS. 33B1-4, a note regarding a treatment plan for the patient was accessed via a pop-up window (element 3) when an icon under the column heading was activated. As depicted in the embodiment of FIGS. 33B1-4, the column heading can contain an icon indicating that a note exists. Element 2 of FIGS. 33B1-4 depicts an icon in the OS column of the VF column that when activated can cause a display of a pop-up window (element 4), which displays a visual field image performed on the patient's left eye. Element 5 of FIGS. 33B1-4 depicts how all of the medications being taken by a patient can be simultaneously displayed using bar graphs and color coding of the present principles.

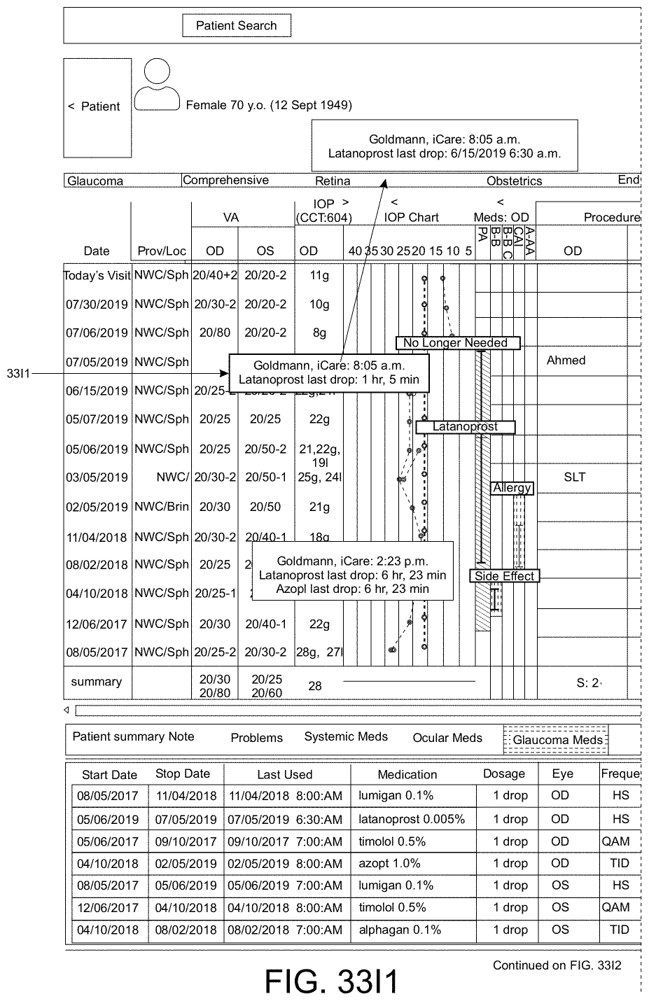

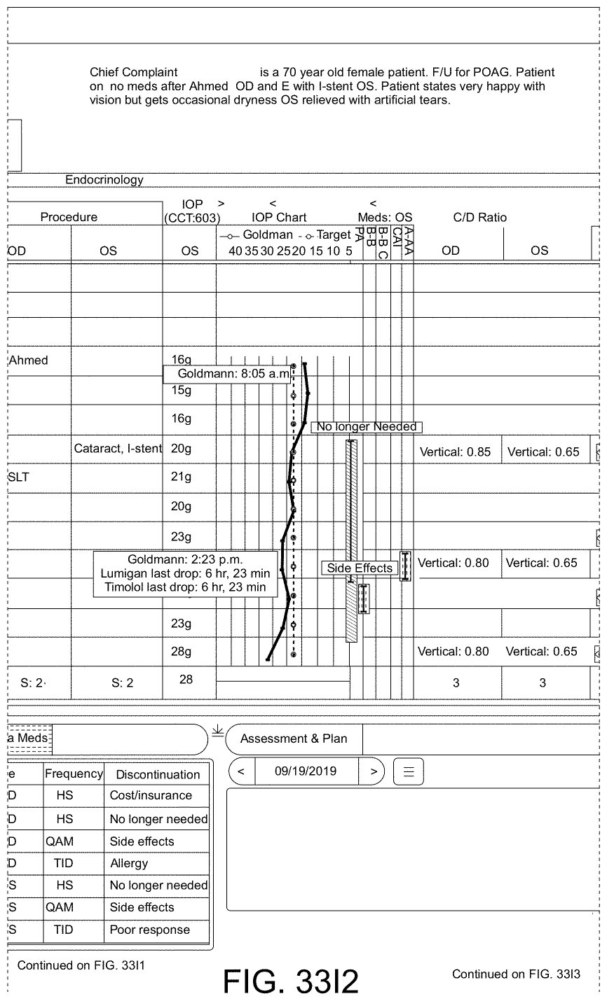

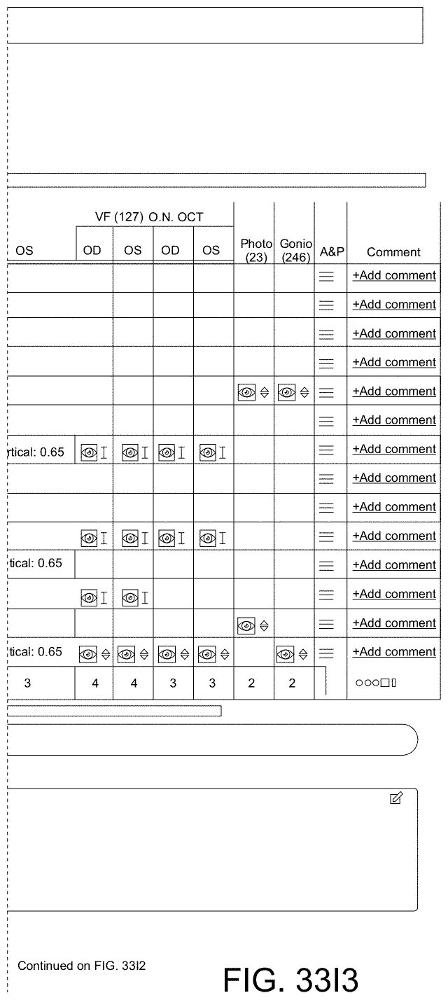

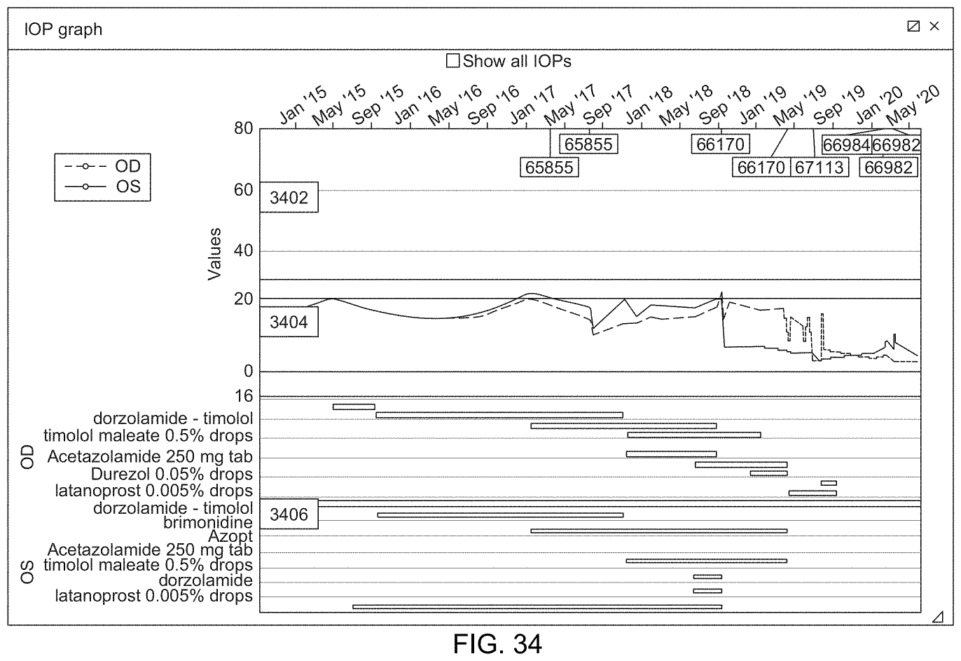

FIGS. 33C1-3 depicts another embodiment of a Medication Management Chart in which icons can be activated to bring up additional information in accordance with another embodiment of the present principles. In FIGS. 33C1-3, element 1 depicts how activating an icon in, for example, a column heading of the Medications Management chart can cause a display of a pop-up window (element 2), which in some embodiments can display another embodiment of a Medication Management chart which displays mediations using horizontal lines and correlates patient well-being data/information (i.e., intraocular pressure) with events that occurred to the patient that would affect the patient's well-being (i.e., the application of medications, surgery, etc.) and with a medication timeline (described in greater detail with respect to FIG. 34). As depicted in FIGS. 33C1-3, using such Medication Management charts of the present principles, a user can make a reasoned estimation of what caused a decline or an improvement in the patient's well-being. Element 4 of FIGS. 33C1-3 depicts that an SLT was performed and element 5 depicts how a small drop in the IOP graph results. In FIG. FIGS. 33C1-3 33C, element 6 depicts that a trabeculectomy surgery was performed and element 7 depicts how a large drop in IOP pressure results. From such results, a user can conclude that the surgery was successful since the goal of a trabeculectomy is to reduce pressure. Element 8 of FIGS. 33C1-3 depicts a medication timeline) and element 9 depicts that the medications were stopped after surgery. As further depicted in FIGS. 33C1-3, a user can select to display information for one eye at a time as depicted in element 10 or for both eyes simultaneously. Element 11 of FIGS. 33C1-3 depicts an alert to remind user to perform a visual field (VF).

FIGS. 33D1-3 depicts an embodiment of the Medication Management Chart in which intraocular pressure, in addition to being listed by number, is also displayed as a vertical line graph, for example as depicted by element 1 in accordance with an embodiment of the present principles. In the embodiment of FIGS. 33D1-3, element 2 displays bar graphs of the medications being taken by the patient. element 3 of FIGS. 33D1-3 depict values of intraocular pressures of a right eye and element 4 displays the corresponding vertical line graphs of the values pointed out by element 3. In FIGS. 33D1-3, element 5 depicts how specific colors can correspond to an assigned medication, in this case orange.

FIG. 33E depicts an embodiment of the Medication Management Chart of FIGS. 33D1-3 in which the control panel can be used to input a reason that a medication has been started or stopped in accordance with an embodiment of the present principles. In the embodiment of FIG. 33E, a drop down menu 33E1 can be used to enable a user to select a reason that a medication has been started or stopped.

FIG. 33F depicts an embodiment of the Medication Management Chart of FIG. 33D in which the correct start and stop dates for a medication in accordance with an embodiment of the present principles. The Medication Management Chart may have one or multiple panels, for example, 3314, 3317, 3320 which may be configured in various ways to display information and/or to accept input from the physician. In one concept of the Medication Management Chart, the Dynamic Health Records (DHP) system displays the medications prescribed to the patient. The start and stop data may be chosen manually by the physician or the DHR system may be able to parse the patient's prior records to obtain this data. A calendar tool such as illustrated as aspect 3340, may be provided by the DHR system as part of the Medication Management Chart to facilitate the manual entry of the dates. In another concept, each medication may be coded in or tagged in some manner such that a visual indication of the medication is provided. As a non-limiting example, each medication may be color coded. The tagging or the coding may be chosen by the physician. As a non-limiting example, the physician may choose different colors for each medication. Further, the DHR system may also provide the capability to tag or color code different medications with the same tag or color. As an example, different medications belonging to the same type or class of medication may be color coded or tagged similarly. The color coding or tagging in some manner medications 3306 and 3310, provides for another concept. In this concept, similarly tagged or color-coded medication may be displayed and correlated along with diagnostic test 3317, clinical exam measurements 3304, 3308 and or any medical services including surgeries and procedures 33I1. As a non-limiting example of correlating medications with one of the items mentioned, clinical measurements, the pressure in each eye may be displayed alongside the medication that was prescribed to that eye as a function of the date when the eye pressure was taken. This is illustrated for example by 3308 displaying the number. The numbers can also be converted into a line graph illustrating the intraocular pressure 3305 and 3309 also displayed alongside the medications that were given to the patient 3306 and 3310 as specifically shown by 3374 and 3370. For this part of the display, each type of medication that was similarly tagged or color coded is displayed in its own column with the start stop dates. A display such as this provides a rapid understanding of the effect of each medication on physiologic parameters as measured by a diagnostic test. For instance, 3366 can display a threshold or target line and the pressure can be seen to be higher 3369 or lower 3371 than the threshold and that can be directly visualized alongside the medications taken in 3310, allowing a user to formulate conclusions about the effectiveness of treatment. The color coding or tagging provides for yet another concept. In this concept because the duration of the medication is visually depicted with the length of the color-coded bars in panels 3306 and 3310, errors in medication orders-can be visually detected. As an example bar graph 3374 can be seen starting with “today's visit” and can be seen extended through the future or next visit 3362 and 3362 can be expanded, for instance, in height to represent a specific time period. The actual length of time in, for instance, days, weeks, or months can be displayed in 3362. Errors in medication duration may be rapidly spotted. As an example, if the medication is supposed to be prescribed for 10 days but the order was entered incorrectly for 10 months, the length of the graph depicting the start and stop date of this medication will be unexpectedly long. Such errors can this be corrected before further processing. To facilitate the entry of the start and stop dates of any medication, the DHR system in addition to mechanism described in 3340, the dates can be set by the user clicking on the panels 3306 or 3309 and medication bar graphs dragged and anywhere along the column by clicking in different cells the medication can be stopped and started for as many times as needed. With this capability, any medication can be started and stopped multiple times to reflect accurately what may have happened in the past and display what the doctor is ordering or prescribing for medications today or in the future. In another concept, the DHR system allows the physician to insert an indicator for the target value of the physiologic parameter. In a non-limiting example, the physician may insert a line that indicates a target pressure in the panel that shows the intraocular pressure. This is illustrated by 3366. The measured pressure may be rapidly compared with the target pressure and conclusions about if the medication or medications is/are working as expected may be intuitively drawn. In a variation of this concept, the DHR system may allow one or multiple indicators to be inserted. In addition, the DHR system may allow the physician to choose the physiologic parameter to be displayed alongside the medications. For instance, by clicking 3315 a drop-down menu displaying a range 3368 can be chosen to set 3366 for a specific target value. 3366 can also in one embodiment be dragged along 3368 and set at the number that the user desires for a particular patient or group of patients. In another variation, multiple different physiologic or other parameters may be selected and displayed alongside each other In a non-limiting example, the blood pressure and the intraocular pressure may be displayed along with the medications the patient takes. The target value indicator leads to another variation of the concept. Here when the physiologic parameter exceeds the threshold value, then the DHR system may trigger one or multiple actions such as but not limited to creating and setting an alert, for instance, as displayed in 3370. In addition, in some embodiments, if a parameter exceeds the threshold value set, for instance, in 3366 a task, message, or alert can be sent to the physician or the care team, displaying a warning message on the screen, automatically scheduling further diagnostic tests, appointments, etc. The behavior when the physiologic or any measured parameter exceeds a threshold value or more generally if the parameter falls outside a preset range this may be programmed or determined by the rule's engine. In another concept, the Medication Management Chart may include a medication ordering panel 3350. In this panel, the physician may order one or multiple medications associated with current visit between the patient and the doctor. The medications may be tagged or color coded as described previously. In addition, the DHR system may also place one or multiple medication order for the future starting and stopping at specific dates. The display of the past, current and future medication orders may be displayed in the medication display panel 3320. This allows the physician to rapidly confirm the current and future orders based on the color coding or tagging. It is to be noted that for the future orders the physiologic parameters will not be shown.

FIGS. 33G1-3 depict an embodiment of the Medication Management Chart of FIGS. 33D1-3 in which both corrected start and stop dates for a medication taken by a patient and incorrect start and stop dates for a medication taken by a patient and listed for example by a 3^{rd} party data provider such as an EMR can be displayed simultaneously in accordance with an embodiment of the present principles. In the embodiment of FIGS. 33G1-3, element 1 depicts a line depicting a start and stop date of a medication being taken by a patient as listed in an EMR. In FIGS. 33G1-3 the line pointed out by element 1 is displayed within a bar pointed out by element 2, which depicts start and stop dates of a medication being taken by a patient as identified by a user. FIGS. 33G1-3 also depicts an alternative embodiment. That is, FIGS. 33G1-3 depicts an orange bar element 4 depicting a medication being taken by the patient. The orange bar depicts start and stop dates element 5 of the medication as listed in an EMR and a black line within the orange bar which depicts start and stop dates of the medication as determined by the user. Importantly and in accordance with the present principles, FIGS. 33G1-3 depicts that more than one stop and stop date can be depicted for each medication in a Medication Management Chart of the present principles. In FIGS. 33G1-3, element 3 depicts the control module that can be used to adjust start and stop dates. In FIGS. 33G1-3, element 8 depicts how when the control module of element 3 is used to change a date, element 9 (5/5/17) the teal color bar graph starts the bar graph at that date.

FIGS. 33H1-3 depicts an embodiment of the Medication Management Chart of FIGS. 33D1-3 in which a user is alerted that a medication being taken by a patient has changed, even if medications are being listed by class and the new medication is of the same class as the old medication in accordance with an embodiment of the present principles. For example, in the embodiment of FIGS. 33H1-3, element 1 depicts a horizontal line in the bar of the medication that is being taken by the patient and that is being changed. element 2 of FIGS. 33H1-3 depicts that before a change the medication being taken by the patient is Lumigan. The line pointed out by element 1 depicts a change in medication and element 3 depicts that the medication being taken after the change is Latanoprost, which is in the same class of medications as Lumigan.

FIGS. 33I1-3 depict an embodiment of the Medication Management Chart of FIGS. 33H1-3 in which a user is able to select a portion of a graph to bring up additional information associated with the graph in accordance with an embodiment of the present principles. For example, in the embodiment of FIGS. 33I1-3, when a user hovers a selection tool (e.g., mouse) over a specific date portion of an IOP graph, a window 33I1 appears displaying to the user information detailing, for example, when and/or where on that particular day an intraocular pressure was measured. Similarly and as depicted in FIGS. 33I1-3, when a user hovers over a specific date portion of a medications graph, the window 33I1 appears displaying to the user information detailing, for example, at what time or how long ago the medication was taken by the patient. In some embodiments, a time between the measurement in the office, for instance, a blood pressure or a pressure of the eye, and how long ago the patient actually took the medication can be measured and displayed, since some medications have a short duration of action and such information would be useful to the user.

In another embodiment and as briefly described above, Medication Management in a Data Command Center in accordance with the present principles exists as a series of intelligent horizontal rows within a correlative graph representing individual medications, classes of medications, categories of medications, or logical groupings of medications, differentiating medications by color or combinations of colors, symbols, and/or text, graphing start and stop dates and times or individual doses, correlated to relevant values and relevant events. In accordance with the present principles, graphical differentiation between medications can consist of individual colors for individual medications, combinations of colors for medications including more than one component, or complex graphical representations. In some embodiments, color standards, such as defined by the American Academy of Ophthalmology, can be used for color coding the medications and/or custom colors can be used. For example, in ophthalmology and with respect to eye care, medications have been assigned in the industry to have a certain color on the eye drop bottle or cap. In some embodiments, these colors can be displayed allowing recognition by the user of the class of medication. For instance, yellow is a beta blocker one of which is Timoptic. In accordance with the present principles, medical care providers who have memorized the color caps can instantly recognize, by viewing a medical records dashboard of the present principles, the class of medication without even seeing the name. Alternatively or in addition, in some embodiments of the present principles a user can identify which generic or brand medication the patient is taking by any means including rolling over the graph and seeing the name of the medication pop up.

FIG. 34 depicts an illustration of a second embodiment of a Medication Management chart that can be displayed in at least a portion of the medical records dashboard of the present principles in accordance with one embodiment. In the embodiment of the present principles depicted in FIG. 34, the medications in the Medication Management chart are color-coded. Illustratively, in the Medication Management chart of FIG. 34, a top section 3402 illustrates dates of relevant events. In some embodiments, such events can include but are not limited to applied medications which can be taken by mouth (orally), given by injection into a vein (intravenously, IV), into a muscle (intramuscularly, IM), into the space around the spinal cord (intrathecally), or beneath the skin (subcutaneously, sc), placed under the tongue (sublingually) or between the gums and cheek (buccally), inserted in the rectum (rectally) or vagina (vaginally), placed in the eye (by the ocular route) or the ear (by the otic route), sprayed into the nose and absorbed through the nasal membranes (nasally), breathed into the lungs, usually through the mouth (by inhalation) or mouth and nose (by nebulization), applied to the skin (cutaneously) for a local (topical) or bodywide (systemic) effect, and/or delivered through the skin by a patch (transdermally) for a systemic effect, surgeries and any other procedures that can affect a patient's well-being.

A second, lower section 3404 of the Medication Management chart of the embodiment of FIG. 34 depicts a line graph correlating the relevant events that can affect a patient's well-being (i.e., the application of medications, surgery, etc.) of the top section 3402 to relevant values of patient well-being data/information (i.e., intraocular pressure) for each of a right eye and a left eye. In the Medication Management chart of FIG. 34, a third, lower section 3406 depicts a horizontal view of medications, which no longer spans a column of appointments, but denotes start/stop dates/times across a linear model. The linear model accounts for dates and key events in the top section of the diagram, such as the application of medications and major surgeries that may also have an effect on the results displayed in the middle section of the diagram. The third lower section 3406 displays an array of medications horizontally in context of the events and factors which can affect results, clearly showing the effect of medications and events on a single, or combination of multiple, tracked values.

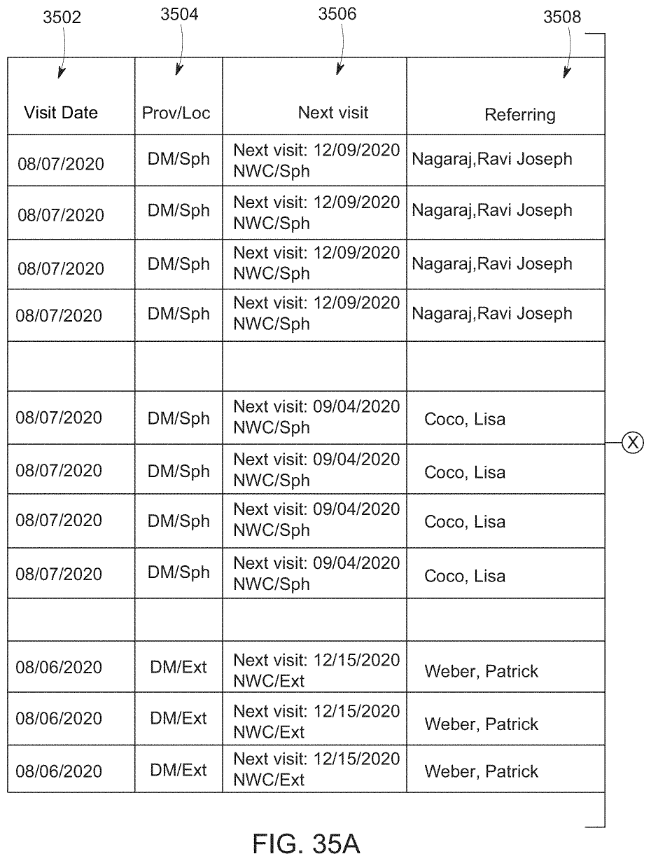

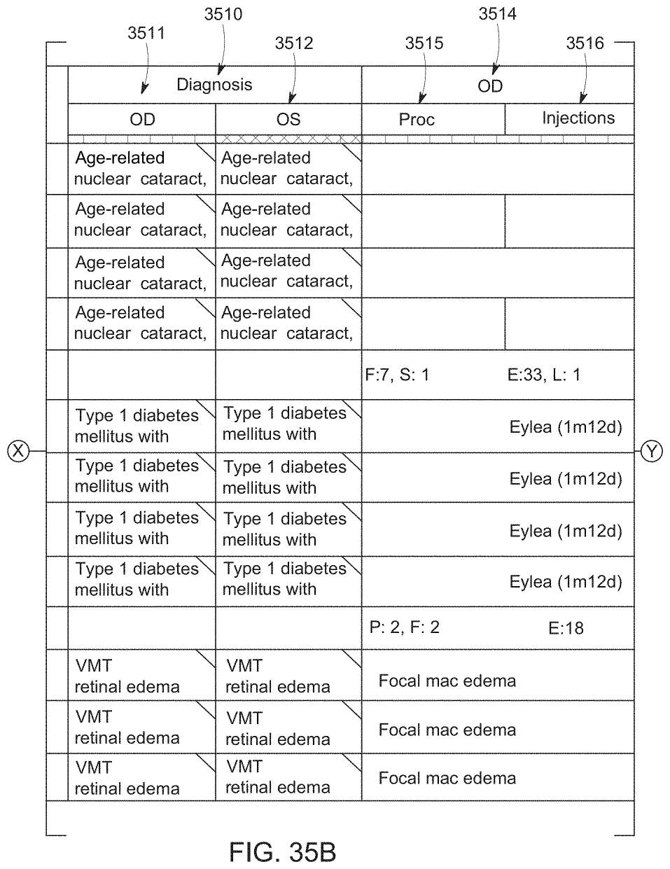

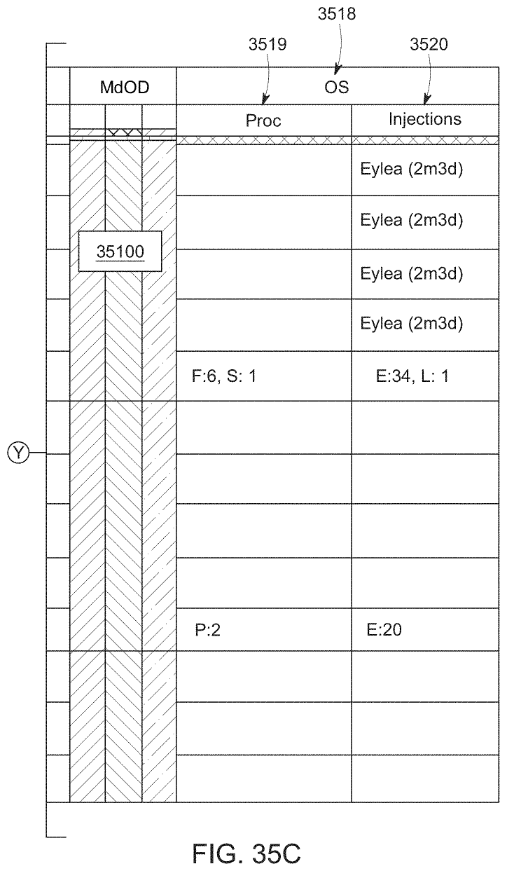

In another embodiment, Medication Management in a Data Command Center in accordance with the present principles exists as a series of intelligent vertical columns representing individual medications, classes of medications, categories of medications, or logical groupings of medications, differentiating medications by color or combinations of colors, symbols, and/or text, graphing start and stop dates and times or individual doses. For example, FIGS. 35A-C depict a medical records dashboard including a third embodiment of a Medication Management chart in accordance with an embodiment of the present principles. That is, the medical records dashboard of FIGS. 35A-C include a report depicting three different patients within a plurality of rows and columns and a Medication Management chart 35100 in accordance with the present principles. In the embodiment of FIGS. 35A-C, the columns of the medical records dashboard include a VisitDate Column 3502 listing the visit date of a patient, a Provider/Location Column 3504, a NextVisit Column 3506 listing a next visit date for the patient, a Referring provider Column 3508 listing the name of, for example, a referring doctor, a Diagnosis Column 3510 including an OD column 3511 and an OS column 3512 including a diagnosis for each of a right and a left eye, a separate OD Column 3514 including a Procedure column 3515 listing procedures performed on a patient's right eye and an Injections column 3516 listing injections performed on the patient's right eye, and a separate OS Column 3518 including a Procedure column 3519 listing procedures performed on a patient's left eye and an Injections column 3520 listing injections performed on the patient's left eye.

In the medical records dashboard of FIGS. 35A-C, the Medication Management chart 35100 depicts a representation of color-coded vertical medication columns as described above with respect to the embodiments of FIGS. 30, 32 and 33.

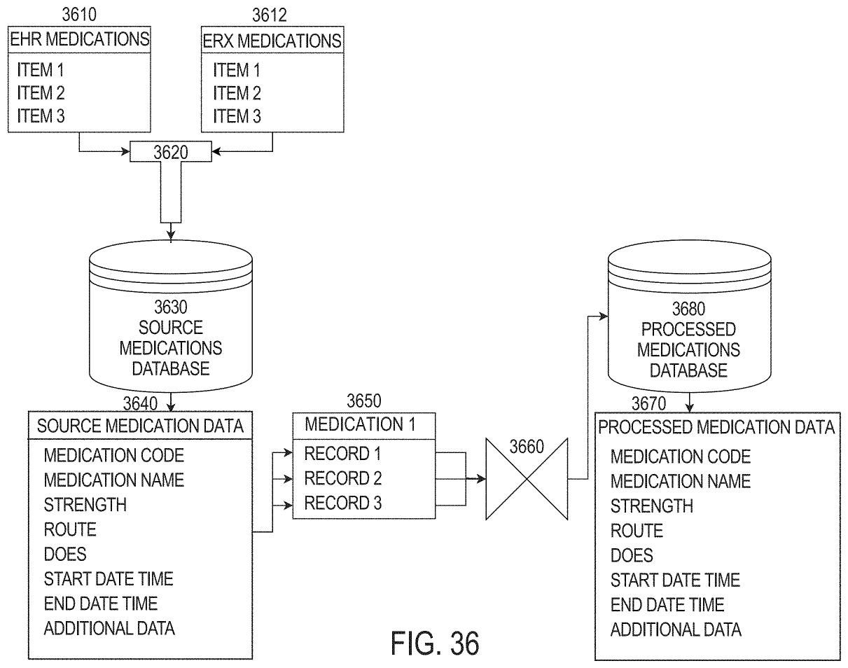

FIG. 36 depicts a high-level workflow diagram of an embodiment of Medication Management in a Data Command Center in accordance with an embodiment of the present principles. In the embodiment of FIG. 36, Medication source data can be stored within an EHR 3610 or eRx platform 3612. In some embodiments, as depicted in FIG. 36, medication data can be imported by an API or other means of digital communication, for example in one embodiment by the integration module 002 of the Data Command center 001 of FIG. 1, and can be compiled into a table 3620 which can be stored in a database 3630. Data from a relevant source stored in the database 3630 can be extracted. Block 3640 depicts an accurate representation of extracted data from the relevant source. The data from the relevant source can then be isolated to at least one specific medication from the source data. Block 3650 of FIG. 36 represents records isolated for a specific medication from the source data. The isolated data can then be processed at 3660 through several intelligent algorithms to surmise a final representation of the view of the specific medication, in some embodiments a longitudinal view. For example, in some embodiments, source data disparities and variance of medication data between sources can be addressed by intelligent algorithms which acquire available data about the medications and data sources and process toward a desired result. Algorithms account for presence if codified data, non-codified data, null values, and other datatypes. Such algorithms can be directed to exporting consistent representations of source data. In some embodiments of the present principles, such algorithms can be applied by the Rules module 004 of the Data Command center 001 of FIG. 1 and can be stored in a means for storage accessible to at least the Rules module 004.

Each medication column or row in a medical records dashboard of the present principles can consist of one or more individual medications as depicted by processed medications data as listed in block 3670, which can be stored in the Processed Medications Database 3680.

In some embodiments, the Display module 006 of the Data Command center 001 of FIG. 1 in accordance with the present principles causes the display of the Medication Management data in a medical records dashboard of the present principles as described above, and specifically in at least one of the vertical, horizontal, and textual embodiments described above and in accordance with individual medications, classes of medications, categories of medications, or logical groupings of medications, differentiating medications by color and/or combinations of colors, symbols, and/or text, graphing start and stop dates and times or individual doses, correlated to relevant values and relevant events as described above.

In some embodiments of at least one of a medical records dashboard and a Medication Management chart in accordance with the present principles, Medication columns and rows can expand, contract, hide, or be display based on a medical care provider's specialty, the identity of a medical care provider and/or a patient, patient conditions, patient procedures, risk factors, diagnostic results, future orders, future appointments, values recorded, values not recorded, calculated values, and absolute values for display, unless otherwise disallowed in accordance with Collapsible Columns and/or Rows that can Collapse and Expand.

In some instances, medication data can be sourced from misleading, unreliable, or inconsistent records reflecting multiple start and stop dates and times for a single medication due to each individual reorder of a medication stopping a prior prescription and starting a new one, or not stopping but adding a new start date and time, and may not reflect actual patient usage of said medication. As such, in some embodiments a Data Command Center via at least of a medical records dashboard and a Medication Management chart in accordance with an embodiment of the present principles enables a user to manually override misleading, unreliable, or inconsistent records to accurately represent medication usage. In such embodiments, each instance of source medication data being altered can be recorded in an audit log to account for data integrity as well as data accuracy. In some embodiments, the source medication data itself is never altered, updated, added, or removed. In some embodiments, updating medication data in any instance of Medication Management in accordance with the present principles reflects in every instance of the Medication Management. For example, editing a stop date and time in a list view of a medical records dashboard can also update the stop date and time in all graphical views. In some embodiments, medication updates can be stored separately from source medication data.

In general, in accordance with the present principles, embodiments of a Data Command Center via at least one of a medical records dashboard and a Medication Management chart of the present principles enable medical care providers to visualize medications, respective start and stop dates, reasons for discontinuation, and enables medical care providers to manage and change a display based on facts able to be confirmed with a patient at a point of care and even with home monitoring devices that can be linked. As described above, in some embodiments each medication can be represented by a bar graph or a linear graph or other visual method or means that in either the vertical direction or in a horizontal direction, a medical care provider can visualize the actual start and stop dates of all relevant medications for their specialty or for that patient all seen simultaneously with any other relevant data that the medications can impact. The medications and any encounters or clinical services or measurements that the patient takes at home or home monitoring devices can all be automatically or manually inputted. The Medication Management chart/Medication Management tool of the present principles can initially be populated by information in the EMR, which may or may not be accurate, or from E-prescribe systems. A medical care provider using, for example a medical records dashboard, can make changes and through a linear bar graph or other means, each column or row can represent a particular medication or class of medication. With all of the patient's medications that are relevant to that medical care provider or the condition being treated, all medications that the patient is taking now or in the past, can be displayed so that medical care providers will know all the medications that the patient has ever taken.

Embodiments of the present principles provide access to whatever information is relevant to the treatment of a patient and is enabled to share this information with all other medical care providers. All medication that can be used to manage a particular condition can all be displayed on a single screen if there is room or collapsed so doctors can visualize other options. In some embodiments, just the columns and/or rows are automatically displayed and other medication alternatives hidden until, through any means, a user accesses hidden patient-related information. In some embodiments, a Data Command Center via, for example at least one of a medical records dashboard and a Medication Management chart of the present principles, can offer clinical decision support in that if there is a set preferred treatment plan or the Data Command Center has programmed proper alternatives that a medical care provider should consider, the medical care provider can start the patient on a particular medication that can be suggested in a blank row or column next to other medications with the name of the suggested medicine.

In some embodiments, each user can move the columns and rows on which the medications are on to a particular section while being able to collapse and expand the entire history of every medication that the patient has taken. Each column or row, depending on whether a horizontal or vertical display is preferable, would be displayed from a start to a stop date and each corresponding date can be listed by office visit of encounter with different medical care providers and or by month, by day, by year, by hour or even minutes especially useful if the patient is hospitalized. In some embodiments of the present principles, a Data Command Center can receive inputs from a user via a user interface on how at least one of a medical records dashboard and a Medication Management chart should be configured to display patient related information from outside sources. For example, in some embodiments patient-related data/information from outside sources can be integrated into the Data Command center 001 via the Integration module 002 of the Data Command center 001 of FIG. 1. Once patient-related data/information is received by the Data Command center 001, the data can be compared to rules to be executed by the Rules module 004, which determine how and if received patient-related data should be displayed. As described above, in some embodiments, at least some of the rules for handling patient-related data/information can be provided to the Rules module 004 of the Data Command center 001 using a user interface. Patient-related data/information can then be caused to be displayed by the Display module 006 on at least the medical records dashboard of the present principles in accordance with the rules of the Rules module 004.

In some embodiments, multiple start and stop dates can exist for a medication based on when a patient admits that they really took the medication. As such, a medication bar graph might appear interrupted because, for example, the same medication might have been taken in 1993 and then re-started again in 2003 or the patient only took the medication for 10 months out of 12 months in a particular year. Such findings can be critical to patient care because if a patient does not take the medication as prescribed it can have an impact on a clinical finding or symptom or disease progression such as high blood pressure. Should a patient have blood pressure measured and suddenly the blood pressure is high, a medical care provider needs to know if it is not that the medication did not work, but perhaps that the patient did not take the medication.

An onset of other medical conditions or interventions such as surgeries or other life events like a death in the family can also be displayed in at least one of a medical records dashboard and a Medication Management chart of the present principles so a medical care provider can determine and take into all the information that can impact the well-being of a patient. As such in some embodiments, a medical records dashboard and a Medication Management chart of the present principles can display clinical findings, measurements, the laboratory findings, and/or whatever the medication impacts a patient's well-being such that a true change in a patient's well-being can be measured accurately and a medical care provider can see visualize the true effects of medications along with other medical services, interventions and life events. By way of example, in the field of ophthalmology there are glaucoma medications, which are pressure medications for the eye. Sometimes just one eye drop will make the pressure go down, sometimes two, three and four different types of drops are needed. Usually medical care providers add a medication if an eye pressure is not controlled to the level desired or if the medical care provider wants to replace one medication with another.

In some embodiments, at least one of a medical records dashboard and a Medication Management chart/tool of the present principles enables a medical care provider to document why a medication was started or stopped or if there has been a reaction to the medication. For instance, if the medication has been stopped because the patient is allergic or cannot afford it, or if it did not work. Such reasons can be input into the medical management tool by selecting the choice by any means such as a drop-down menu or through voice recognition software or any means. The information can then be displayed on a bar or line graph of that particular medication and either be permanently displayed or accessed via an icon or other access point.

In various embodiments of a medical records dashboard having a medication management tool (such as displayed in FIGS. 30A, 30B, 30C, 30D, 30E, 30F, 30G, 30H, and 30I), in addition to a laboratory or clinical finding, there can be included an option to input information regarding procedures performed on a patient. For example, for a particular patient, a surgical procedure might be the reason there has been a sudden change in the well-being of the patient. For instance, there are some glaucoma pressure surgeries which will reduce the pressure and have the same effect as a medication or a laser surgery that might cause a pressure to be lower. It is important that a medical care provider have the option to view what procedure were performed on a patient to determine if a procedure might also have had an effect on the clinical finding, symptoms or disease progression on which the medication can also have an impact. Perhaps it is not the medication that is working, maybe it is the surgery.

Embodiments of the present principles are fully adjustable for all types of conditions, such as high blood pressure, diabetes, rheumatological diseases, and all types of cancer. All of these conditions have certain laboratories and clinical measurements that are taken either at the patient's home or from a testing center or on each visit with a medical care provider (i.e., doctors often record weight and blood pressure of the patient, etc.). In addition, a medical care provider can be enabled via at least on of a medical records dashboard and a Medication Management chart of the present principles to now E-prescribe or place an order for a new medication or cancel a drug. As such, by ordering a next medication, a medical care provider can instantly visualize what is being ordered as the new order can be displayed as a future medication. In such embodiments, a new column or row can appear with, for instance, a new bar graph because the medical care provider is now ordering a new medication.

A Data Command Center of the present principles enables a medical care provider to determine if incompatible medications or procedures have been ordered and/or scheduled. For example, in some embodiments, upon the visual display of ordered medications and/or procedures in at least one of a medical records dashboard and a Medication Management chart of the present principles, a medical care provider, by looking at the display, can visually determine through his/her experience and training that incompatible medications and/or procedures have been ordered or scheduled. Alternatively or in addition, in some embodiments a Rules module, such as the Rules module 004 of the Data Command center 001 of FIG. 1, can be programmed to recognize incompatible medications and/or procedures. As such, when patient-related data/information containing incompatible medications and/or procedures is received or when incompatible medications or procedures have been ordered or scheduled by, for example, a medical care provider using for example at least one of a medical records dashboard and a Medication Management chart of the present principles, the Rules module 004 can cause an alert to be displayed by, for example, the Display module 006, the alert intended to bring to a user's attention that incompatible medications and/or procedures exist. In some embodiments, if such a condition exists, a pop-up can appear to enable a medical care provider to re-do their order and make sure the order is corrected.

In some embodiments, multiple medication graphs can be shown independently or on for example, at least one of a medical records dashboard and a Medication Management chart of the present principles, such that a user is able to compare different reporting of the same medications. For example, in some instances patient-related data from an EMR can be inaccurate. However, it is advantageous for a medical care provider to know what has been documented, even if inaccurate. Embodiments of a medication management tool of the present principles can display two graphs, a first displaying what is actually documented in the EMR and a second displaying patient-related data that has been corrected by a medical care provider. In such embodiments, a medical care provider is enabled to check patient-related information from an EMR for accuracy.

In the medical field, medical care providers, such as doctors, use drug categories according to the affects they have on the human body. Many types of categories can be classified on the basis of chemical nature of the drug. The term of the drug or medication is used for diagnosing, curing, or treating a disease. Drugs classification can include but are not limited to a Chemical nature of the drug, Symptoms or diseases for which they are used (i.e., antihypertensive drugs), Organ system affected, Generations of drugs, such as antimicrobials or oral hypoglycemic agents, Receptor theory, Duration of action, and method of administration. Embodiments of a medical management tool in accordance with the present principles enable medical care providers to display all of a patient's medications by classification by, in some embodiments, selecting from a menu whatever classification method is most intuitive to the medical care provider as the medical care provider is treating the patient. By way of example, in the case of a subspecialist, like an ophthalmologist, the doctor might just want to know all medications of the eye, so the organ system affected is the eye. For instance, in the eyes category of disease can be glaucoma, which includes pressure control in the eyes. For glaucoma, there is a group of medications that control pressure in the eyes. Currently, there are eight classifications. In addition, there is macular degeneration disease or diabetic macular edema disease and there are classifications for those diseases as well. A medical care provider can decide to display, on a single display, either all of the ocular medications that the patient is taking singly or in categories. Alternatively or in addition, a medical care provider can select to display medication by symptoms of the disease, such as the anti-hypertensive medications.

It can also be helpful to a medical care provider to know if a patient is taking an originally prescribed brand of the medication or if the patient is taking a generic medication. Embodiments of a medication management tool of the present principles provide a means for listing whether a patient is taking an originally prescribed brand of the medication or if the patient is taking a generic brand. A difference between the two brands of medication is that one might cost a significant amount more than the other and some can work a little differently and not be as affective. Medical care providers need to know whether the patient is taking a brand name or a generic. Some insurance companies will only pay for certain brands or generics, and mandate that medication be taken. Some medications will have a copay by the patient and the patient has to pay additional money. It can be critical that medical care providers also note cost to patients and to the insurance companies, so that medical care providers can control health care dollars.

In some embodiments of a Medication Management tool of a Data Command Center of the present principles, the Medication Management tool can make suggestions in regards to using a less expensive generic medication and in some embodiments can compare medication and procedure recommendations made by a user/medical care provider against what a patient's insurance will allow. For example, in some embodiments information regarding generic medications that can be substituted for brand name medications can be stored in a storage means accessible to, for example, the Rules module 004 of the Data Command center 001 of FIG. 1. As such, when a user/medical care provider prescribes a medication using the Medication Management tool and/or a medical records dashboard of the present principles, the Rules module 004, via for example the Display module 006, can cause a display of suggested generic medications, in some embodiments in a pop-up window, that can be prescribed to a patient in place of the brand name medication. Similarly, in some embodiments information regarding what medications and procedures can be authorized by a patient's insurance company can be stored in a storage means accessible by, for example, the Rules module 004 of the Data Command center 001 of FIG. 1. As such, when a user/medical care provider prescribes a medication or schedules a procedure using the Medication Management tool and/or a medical records dashboard of the present principles, the Rules module 004 can compare the information regarding what a patient's insurance company will allow and what medication the user/medical care provider has prescribed or what procedure was scheduled to determine if the patient's insurance company will allow the medication and/or procedure. If the Rules module 004 determines that a prescribed medication and/or scheduled procedure is not allowed by a patient's insurance company, the Rules module 004, via for example the Display module 006, can cause a display of an alert to the user/medical care provider to alert the user/medical care provider that a prescribed medication and/or scheduled procedure is not allowed by the patient's insurance company. In some embodiments, information regarding what medications and procedures can be authorized by a patient's insurance company can be stored in a storage means accessible to the Rules module 004 of the Data Command center 001 of FIG. 1.

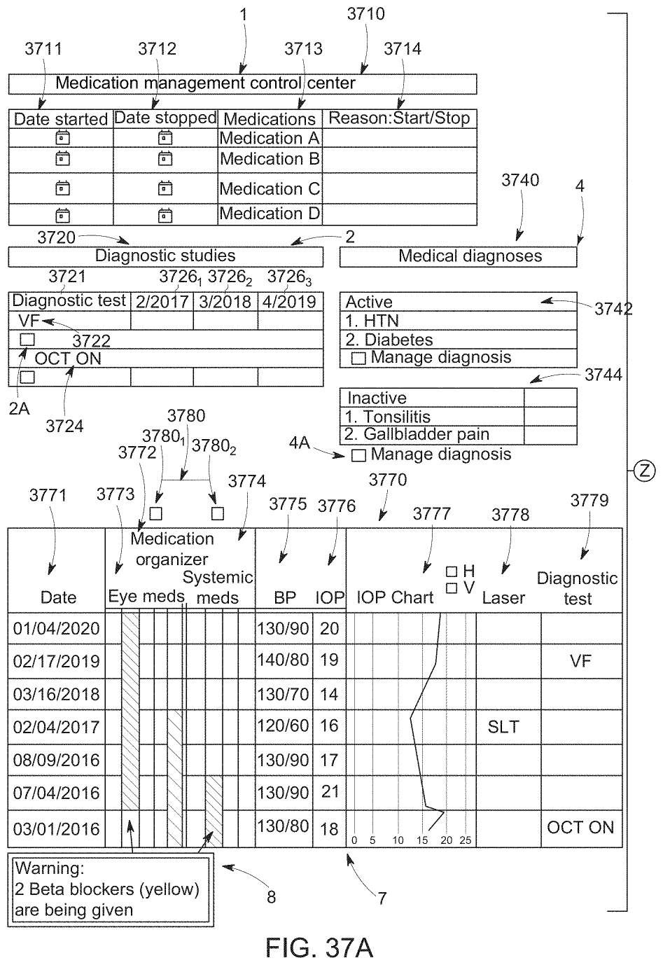

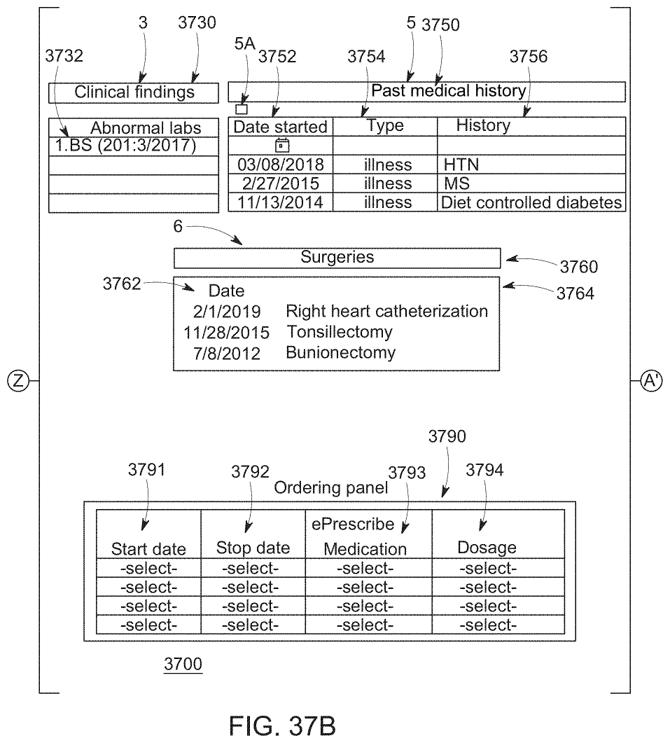

FIGS. 37A-B depict an exemplary embodiment of a Medications Management chart/tool 3700 which does not use rows or columns in accordance with an alternate embodiment of the present principles. Block 1 of the Medications Management chart/tool 3700 of FIGS. 37A-B depict a control panel 3710, which can be used to configure the bar graphs of block 7 and 8 described in greater detail below. The control panel 3710 of FIGS. 37A-B illustratively comprises a date started column 3711, a date stopped column 3712, a medications column 3713 illustratively listing medicines A, B C and D, and a start/stop reasons column 3714.

Block 2 of the Medications Management chart/tool 3700 of FIGS. 37A-B depict a diagnostic studies menu 3720, which can be used to list diagnostic studies performed on a patient. The diagnostic studies menu 3720 of FIGS. 37A-B illustratively comprises a diagnostic test column 3721, including a VF row 3722 and an OCT ON row 3724, and three date columns 3726₁, 3726₂ and 3726₃. In the diagnostic studies menu 3720 of FIGS. 37A-B, by hitting 2A, a user can pull up an individual test or get thumbnails of the tests performed on a patient. Block 3 of the Medications Management chart/tool 3700 of FIGS. 37A-B depicts a clinical findings menu 3730, which can be used to list clinical findings on a patient. The clinical findings menu 3730 illustratively comprises an abnormal labs column 3732 for listing abnormal laboratory findings for a patient.

Block 4 of the Medications Management chart/tool 3700 of FIGS. 37A-B depict a medical diagnosis menu 3740, which can be used to list medical diagnosis made by a user for a patient. As depicted in the embodiment of FIGS. 37A-B, the medical diagnosis menu 3740 can be divided into active 3742 and inactive 3744 diseases. In the embodiment of FIGS. 37A-B, the various diagnoses or conditions of the patient can be managed on the screen by clicking 4A. Block 5 of the Medications Management chart/tool 3700 of FIGS. 37A-B depicts a past medical history menu 3750, which can be used to list conditions that affect the well-being of a patient. As depicted in the embodiment of FIGS. 37A-B, the past medical history menu 3750 illustratively includes a date started column 3752, a type column 3754 and a history column 3756. In the embodiment of the past medical history menu 3750 of FIGS. 37A-B, by hitting 5A, a user is able to edit any of the information in the past medical history menu 3750.

Block 6 of the Medications Management chart/tool 3700 of FIGS. 37A-B depicts a surgeries menu 3760, which can be used to list surgeries performed on a patient. As depicted in the embodiment of FIGS. 37A-B, the past medical history menu 3750 illustratively includes a date started column 3752, a type column 3754 and a history column 3756.

Block 7 of the Medications Management chart/tool 3700 of FIGS. 37A-B depicts a dashboard 3770. The Dashboard 3770 of the Medications Management chart/tool 3700 of FIGS. 37A-B illustratively comprises a date column 3771, a medication organizer column 3772 including an eye medications column 3773 and a systemic medications column 3774, a blood pressure (BP) column 3775, an intraocular pressure (IOP) column 3776, an IOP chart/graph column 3777, a laser column 3778, and a Diagnostic test column 3779. The Dashboard 3770 of the Medications Management chart/tool 3700 of FIGS. 37A-B illustratively further comprises a respective ordering panel selection block 3780₁, 3780₂ for each of the eye medication column 3773 and the systemic medications column 3774. When a user selects either of the ordering panel selection blocks 3780₁, 3780₂, an ordering panel 3790 such as an E-prescribed panel is displayed that enables the user to place an order, which can include prescribing a medicine, and comes up in a way that does not block the entire view. The ordering panel 3790 illustratively comprises a start date column 3791, a stop date column 3792, a medication column 3793, and a dosage column 3794.

In the Dashboard 3770 of the Medications Management chart/tool 3700 of FIGS. 37A-B, the eye medication column 3773 and the systemic medications column 3774 include bar graph representations of medications associated with the treatment of a patient's eye. Illustratively, in the Medications Management chart/tool 3700 of FIGS. 37A-B, a user is being warned in block 8 that two beta blockers, depicted as yellow bars, are being given to the patient. Since there is a relationship between the two, the user needs to know.

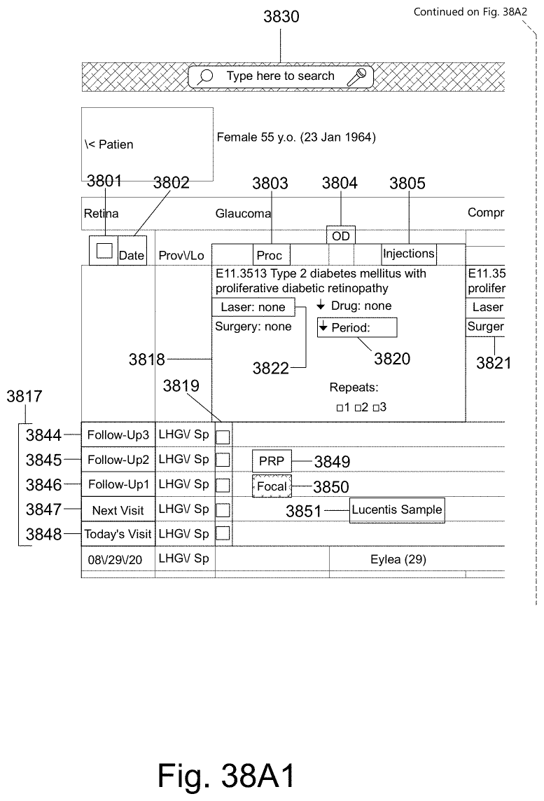

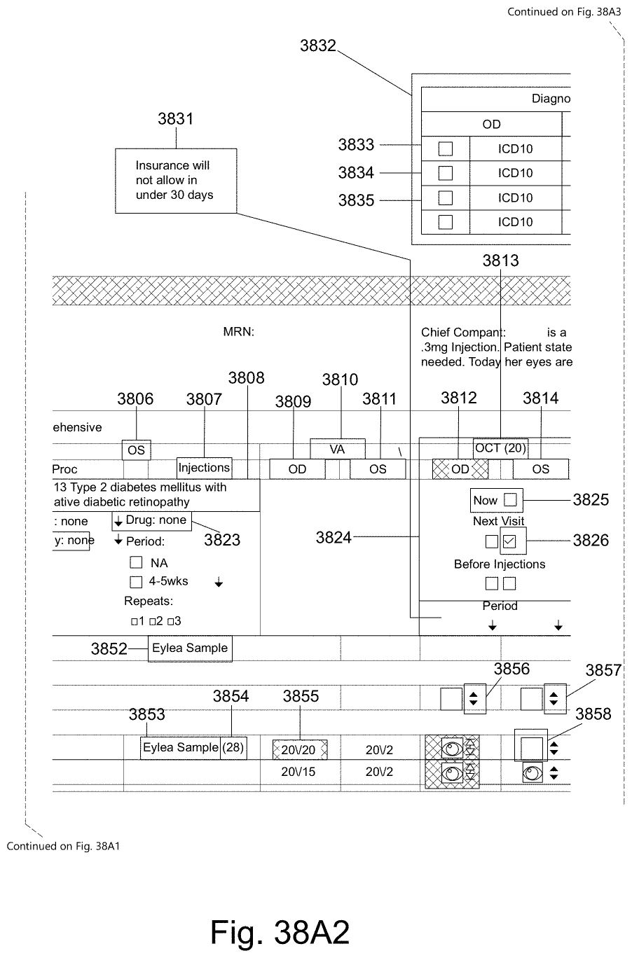

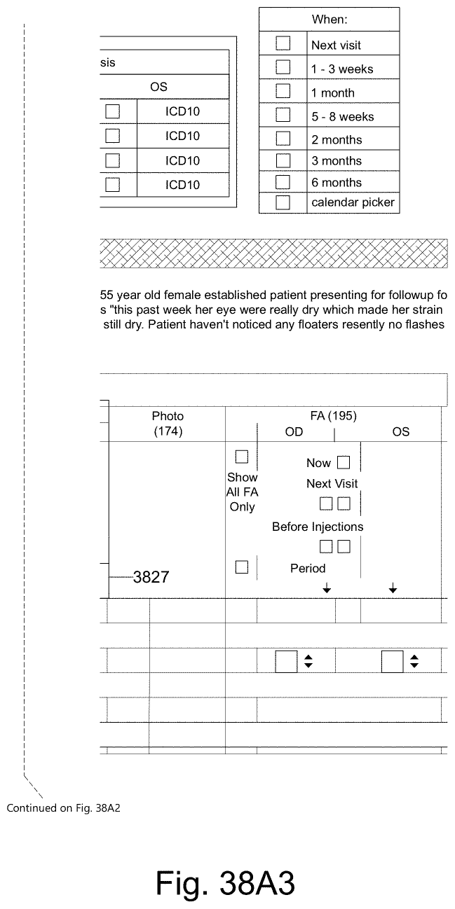

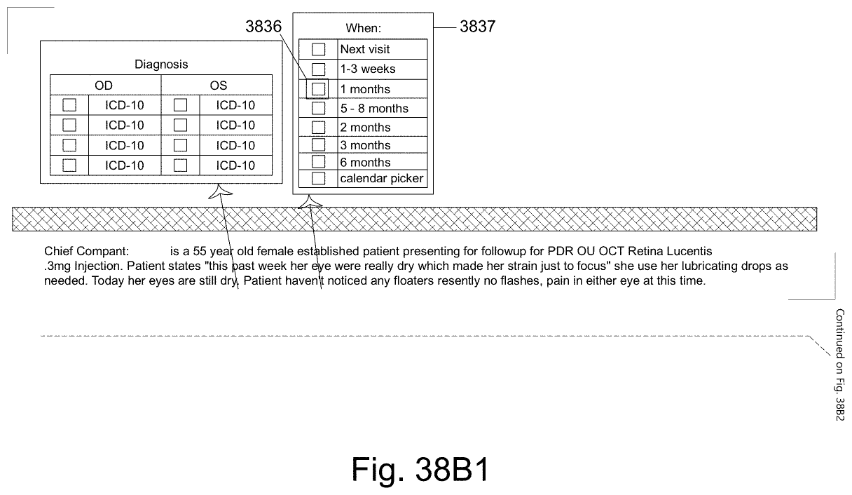

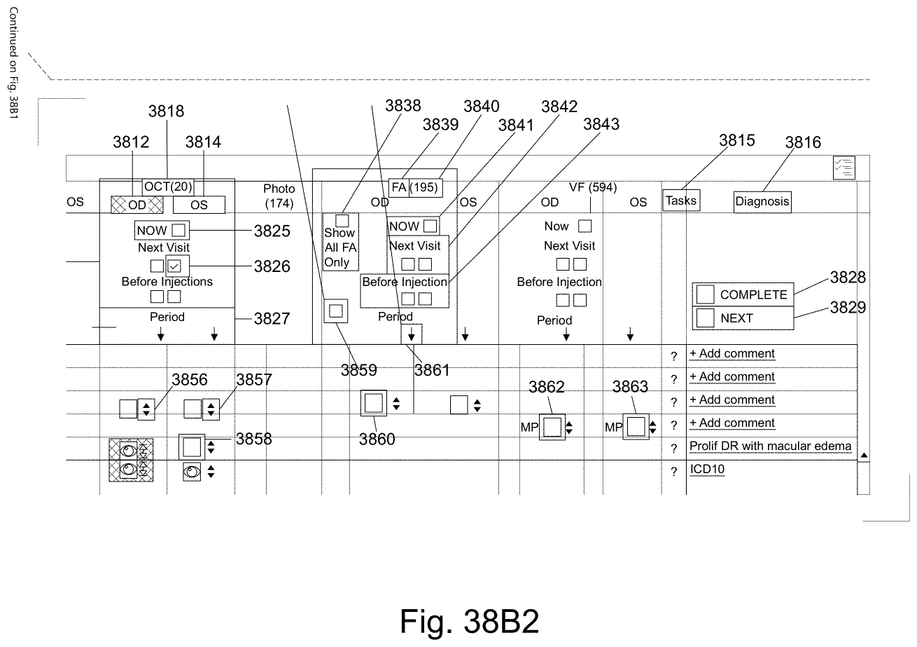

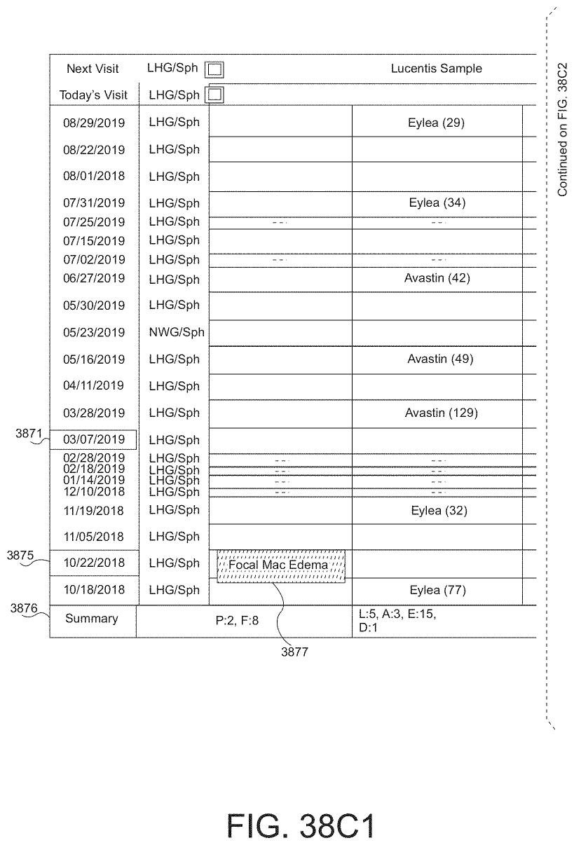

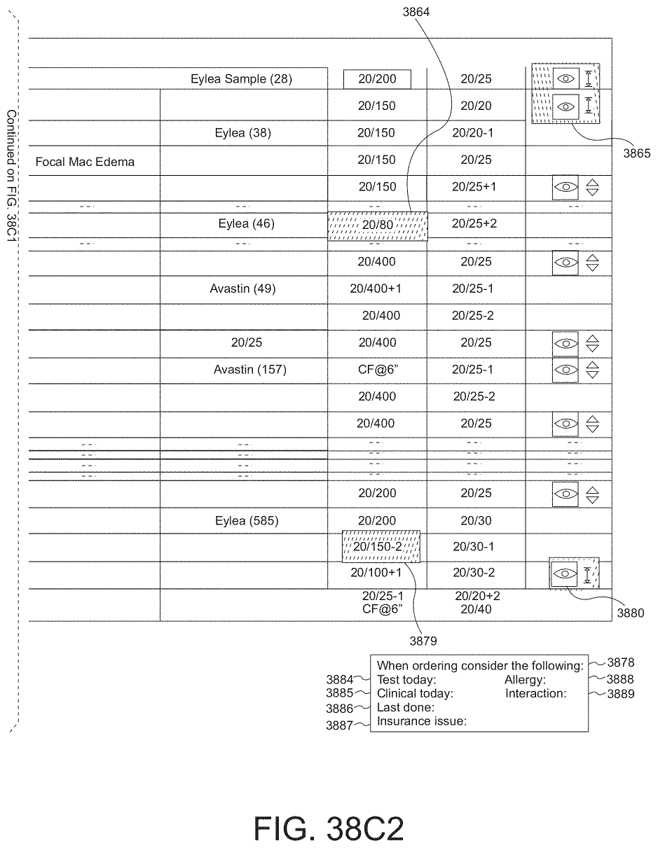

FIGS. 38A1-3, B1-2, C1-2 and D, hereinafter collectively referred to as FIG. 38 depicts an embodiment of a medical records dashboard of a Data Command Center in which a user/medical care provider is enabled to place orders in context with other relevant patient data/information, so as to enable the user/medical care provider to see the future orders in context and confirm that the orders submitted are in fact what the user/medical care provider intends in accordance with the present principles. The details of FIG. 38 are being presented as FIGS. 38A1-3, B1-2, C1-2 and D (collectively referred to as FIG. 38 below) to enable more clear visualization of the features of the embodiment of FIG. 38. In some embodiments, a column of the medical records dashboard can be expanded by selecting the column. For example, in FIG. 38, the column 3801 is expanded as depicted by window 3818 to allow for room for placing an order. In some embodiments, the window 3818 can comprise a pop-up panel for placing orders. In FIG. 38 cells 3804, 3818, and 3839 depict examples of cells displayed in the medical records dashboard that are in the line and above corresponding columns that identify that orders have been made and/or enable the placement of new orders. For example, cell 3804, as depicted in expanded FIGS. 38A1-2 correspond to a panel that can be used for placing orders for a right eye (OD) 3803 and is located directly above procedures 3877 performed in the past for the right eye (OD). Another example is the ordering panel 3839 which is above the FA column. Illustratively, in the FA column, a user can identify when the last time something was performed, enabling a user/medical care provider to determine if it is time to order a new procedure. From the medical records dashboard of FIG. 38 it can be determined from row 3871 that the last FA 3873 was done (3/7/19) and the FA in the header cell 3839 depicts that the last FA, was 195 days ago as depicted in cell 3840. In the embodiment of the medical records dashboard of FIG. 38, a user is enabled place an order while visualizing a particular CPT codes (diagnostic test, procedures, office visit, etc.) ordered in the past and can visualize how often it was performed, when the last time it was performed. In FIG. 38, an illustrated FA 3839 in row 3876 reports the total number of times the item to be re-ordered was previously performed. In the example of an FA shown in cell 3881 of FIG. 38 an FA was performed seven times, in the right eye. In FIG. 38, cell 3882 depicts that an FA was performed five times in the past in the left eye.

As described above, in the embodiment of FIG. 38, expansion of an ordering panel can occur in both in height and in width. In some embodiments, to enable the expansion of an ordering panel, columns that are considered by a user/medical care provider as unnecessary can be collapsed to enable viewing expanded ordering panels in context with information deemed necessary. For example, a clinical measurement, such as vision measurements in columns 3810, 3809, 3811 can be collapsed if a user determines such information is not currently needed, enabling horizontal expansion of ordering panels. In some embodiments, the ordering panels 3839, 3813, 3804 can widen when the user/medical care provider clicks on them to then place an order to enable a user/medical care provider to simultaneously visualize, using a single display, data relevant to the newly placed orders. In accordance with embodiments of the present principles, the display 3830 remains interactive during the display of the ordering panels to enable a user to scroll down to see past FA performed, for example, prior to the 10/18/18 row 3830 of FIG. 38 depicts a search mechanism enabling the user to type in or ask any questions and whatever rows with the relevant data would be the rows visualized with other rows collapsed or hidden. For instance, Cell 3881 depicts that seven FA were done yet only the FA done 03/07/19 3871 is displayed in this single view but all seven dates of service when 3839 were performed, the tool would display those rows for instance clicking on cell 3881 to display may be important as a user is ordering a new FA. In this way, as the user orders, for example, an FA, the user is able to visualize what was done in the past.

In the embodiment of FIG. 38, an FA can be ordered by activating ordering panel 3839 to expand the panel. The user could then decide if what the users want displayed in hat column, 3839 is just the most recent FAs, in FIG. 38 depicted by cells 3874 and 3873 in row 3871. A user, alternatively, could scroll down and find the other FAs for the earlier dates or by clicking on cells 3881 for the right eye or 3882 for the left eye or the tool can be programmed to show all the past dates that the particular test or procedure was performed. Embodiments of the present principles enable a user to search as depicted in cell 3830 or to scroll to display the seven FA rows. In some embodiments, all of the rows and dates of service can be collapsed to make room to display today's visit in, for example, cell 3848. That is, because an action is being performed by a user, a current row can remain visible. A next visit then can be displayed in a follow up cell 3847 and a future order cell 3846 can become visible, as the user places orders for different future dates of service with row popping-up as user places orders for each future visit. Alternatively, in the embodiment of FIG. 38, a user can prioritize the visualization of rows/cells depicting when FA was performed and collapse other rows/cells by clicking on icon 3852, which enables a collapsing of all rows except the rows when an FA was performed.

In the embodiment of the medical records dashboard of FIG. 38, if a user/medical care provider wants to double check if an order placed is proper and wants to see a related study itself, the user/medical care provider can select cells 3874 and a respective image can be displayed in 3869 so the ordered study can be interpreted in context of all other information being presented in the medical records dashboard. The user/medical care provider can view directly, an image or even choose multiple icon images of, for example, the FA. The ordering panels that are displayed when selected (i.e., 3801 or 3839) can be customized by specialty, for example in FIG. 38 for a retina specialist. In the embodiment of FIG. 38, a retina specialist can perform injections on a patient, as such in accordance with some embodiments of the present principles, the retina specialist can be presented with an option to perform the FA before an injection, 3843. In such embodiments, the injections are not hidden and can be seen in column 3807. The scheduling for the test (e.g., FA 3839) can then be accomplished by activating cell 3861, at which point an option for selection can be displayed (i.e., 3837) and the user can select form a pull down menu how far in the future (illustratively one month 3836) to order the study.

In some embodiments, a Rules module, such as the Rules module 004 of the Data Command center 001 of the embodiment of FIG. 1, can be configured to determine if a patient's insurance company will disapprove of ordered studies and can further be programmed to determine if a patient has an aversion to an ordered study and can cause a display, for example via the Display module 006, of an alert or information window on the medical records dashboard to inform a user/medical care provider of such instances.

In the embodiment of FIG. 38, cell 3813 can be used to order an OCT test. For example, cell 3814 can be selected by a user to select a left eye then OCT (OS), cell 3826 selects a next visit, and cell 3827 can be selected for choosing a time period. In some embodiments, a Rules module, such as the Rules module 004 of the Data Command center 001 of the embodiment of FIG. 1, can have access to a storage means containing rules for scheduling tests (i.e., certain tests have rules for how often the tests can be performed on a patient) and the Rules module 004 be configured to determine if tests/studies have been improperly ordered. In such instances, the Rules module 004 can cause a display, for example via the Display module 006, of an alert or information window on the medical records dashboard to inform a user/medical care provider that perhaps a test/study has been improperly ordered via, for example, a pop-up window 3831.

In the embodiment of FIG. 38, a user/medical care provider is enabled by the medical records dashboard to select a reason for ordering a test or procedure. In the embodiment of FIG. 38, cell 3859 can provide a menu providing options for a user to select for inputting reasons for ordering a test or procedure. In some embodiments, such options provided to a user in cell 3859 can be pre-programmed. Alternatively or in addition, a Rules module, such as the Rules module 004 of the Data Command center 001 of FIG. 1, can be programmed to monitor data/information related to a patient including. but not limited to, previous diagnosis made, previous tests ordered, previous procedures ordered and respective reasons for ordering the tests and procedures, and the Rules module 004 can be configured to learn, for example, through machine learning and/or artificial intelligence means to determine at least a best reason for ordering tests and procedures depending on relevant patient information. In such embodiments, the Rules module 004 can cause the display, for example via the Display module 006, of most logical reasons for ordering a test or procedure in, for example, a drop down menu provided by cell 3859 of the medical records dashboard of FIG. 38. For example, the Rules module 004 can be aware of what CPT codes can be associated with ICDs for a particular patient for which test and/or procedures are being ordered and the most logical diagnostic codes can be presented, for example in cells 3833, 3834, and 3835. In the embodiment of FIG. 38, if a user/medical care provider is unsatisfied with the reasons for ordering provided in, for example, a drop down menu provided by cell 3859, the user/medical care provider can select cell 3832 to see more options or to insert a reason for ordering.

In the embodiment of the medical records dashboard of FIG. 38, a user/medical care provider can select using for example cell 3814, for which eye a test/study/procedure is to be ordered. A diagnosis and information regarding what is ordered is displayed in rows 3848, 3847, 3846, 3845, and 3844, collectively 3817 depending on when the order is scheduled. The user can visualize the order, then by any means, confirm it is correct, by selecting cell 3814. The user/medical care provider is able to confirm everything in a row displayed is correct as visualized and confirm the order for that entire future date of service by selecting cell 3819. In the embodiment of FIG. 38, a user can be informed of what is being ordered by displaying in a corresponding row, an empty icon or empty box, for example 3960 of 3846. If the doctor wants to also order an OCT in the right eye, cell 3812 can be selected and the process repeated.

In the embodiment of the medical records dashboard of FIG. 38, cell 3870 shows all past encounters of relevance in which a user can view all of the information by scrolling or viewing on a single display. Cell 3870 keeps track of every encounter and a date and/or time of the encounter, any medical service, ICD 10 with diagnosis or clinical information or procedural information. Cell 3876 includes a summary of how often orders have been placed in any period of time. Row 3848 depicts information regarding “today's visit.” Today's visit can be live and in real time in some embodiments. Clinical information, i.e. in this example vision (VA), can be displayed as it is input in corresponding columns 3810, 3809, 3811. Column 3807 depicts what is to be done today and in the embodiment of FIG. 38 depicts an injection with medication 3853, “Eylea sample.” Cell 3854 of FIG. 38 depicts that the procedure was to be performed 28 days ago, which, as described above, can be checked by the medical records dashboard for compliance.

In the embodiment of FIG. 38, row 3848 shows under column 3814 an OCT and an empty box 3858. Such configuration can indicate to a user/medical care provider that the ordered procedure/test/study has not yet been performed because in the embodiment of FIG. 38 the order was scheduled in “today's visit,” meaning that the user/medical care provider placed the order today. In comparison, cell 3866 is filled in because on the last visit the test had been performed.

In some embodiment of the present principles, an appearance of the cells of the medical records dashboard can be altered to distinguish/highlight the information in the cells. For example, in the embodiment of FIG. 38, cells 3860, 3862, 3863, 3850, 3851, 3849, 3852 are examples of cells containing future orders. In some embodiments, cells can be made lighter or darker to differentiate past versus future actions/orders. In addition and for example, row 3848 of “today's visit” can be made blue. Even further, in some embodiments of the present principles, icons or markers can be included in cells/rows/columns of the medical records dashboard to enable a user to make a determination of the information included in a cell just by looking at the icon/marker. In some embodiments, the icons/markers can also include color to further distinguish between information represented by the icon/marker. For example, icons 3865, 3880 can be shown as colored indicators to indicate a status of the condition of a user's eye described in cell 3867 and 3872.

In the embodiment of the medical records dashboard of FIG. 38, related cells can be highlighted to call a user's attention to relevant patient data when placing an order. For example, cell 3822 enables a user to order a laser. Cell 3850 depicts that a focal laser is to be ordered in the future. In conjunction, cell 3877 can be highlighted to alert the user/medical care provider of the last time a similar focal laser was done. In addition, cell 3879 can be highlighted to alert a user what the vision of the patient was at the time of the last laser performed Oct. 22, 2018. As such, a user/medical care provider can take into account related patient data as they place an order for a focal laser in cell 3803 as displayed in cell 3850 for a follow up row 3846, as scheduled by any means, by way of example, within the pop-up window 3803. By noting a previous condition of the vision of a patient in accordance with the present principles, a user can identify if a patient's condition is getting better, worse or remaining the same. For example, in the embodiment of FIG. 38, icons 3865 and 3897 show red indicators to indicate a worsening of a condition of a patient's eye.

In another example of placing orders, as described above a medical records dashboard of the present principles, via for example a Rules module, can be aware of what the most common ICD10 might be (i.e., via cell 3832) when ordering. Cell 3842 depicts a user selecting a box and an order can be directly linked to the box the user selects, which can be displayed in a pop-up window as depicted in cell 3827, 3861, and 3836. The future encounter can be selected and confirmed in cell 3828 and the next encounter ordered in cell 3829, which in this embodiment means another date of service in the future is to be ordered and displayed, and the process starts again. This functionality enables users/medical care providers to confirm future orders by reviewing available patient related data being simultaneously displayed in the medical records dashboard.

As depicted in the embodiment of FIG. 38, the medical records dashboard can include panel 3878 for assisting a user/medical care provider in placing an order. That is, in some embodiments, when a user/medical care provider is placing an order, panel 3878 can be presented to the user/medical care provider to present to the user/medical care provider a list of things that the user/medical care provider should take into considerations when placing an order. In the embodiment of FIG. 38, the panel 3878 includes considerations such as 3884 a diagnostic test that was done today or on a previous visit, 3885 clinical findings found today, 3886 a last time the same or similar test/study/procedure was done, 3887 insurance issues, 3888 allergy concerns, and 3889 possible interactions with other tests and/or medications. A Rules module, such as the Rules module 004 of the Data Command center 001 of FIG. 1, can be configured to monitor such considerations and alert a user/medical care provider if a problem is determined. Although the panel 3878 of FIG. 38 depicts a specific listing of considerations in panel 3878, in alternate embodiments, the considerations listed in panel 3878 can change dependent upon what is being ordered.

As depicted in the embodiment of FIG. 38, the medical records dashboard can include panel 3893 for assisting a user/medical care provider in placing orders. That is, in some embodiments, when a user/medical care provider is placing an order, panel 3883 can be presented to the user/medical care provider to present to the user/medical care provider an order summary. In the embodiment of FIG. 38, the panel 3883 includes a listing of 3890 what is being ordered, 3891 a last date the same procedure was performed on the patient, and 3892 any relevant clinical information. Although the panel 3883 of FIG. 38 depicts a specific listing of related order information in panel 3883, in alternate embodiments, the order related information listed in panel 3883 can change dependent upon what is being ordered.

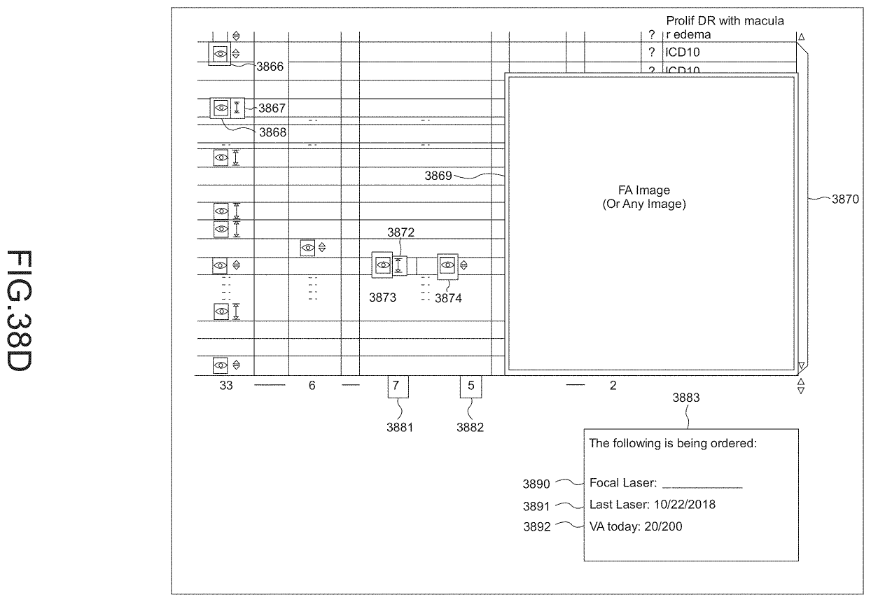

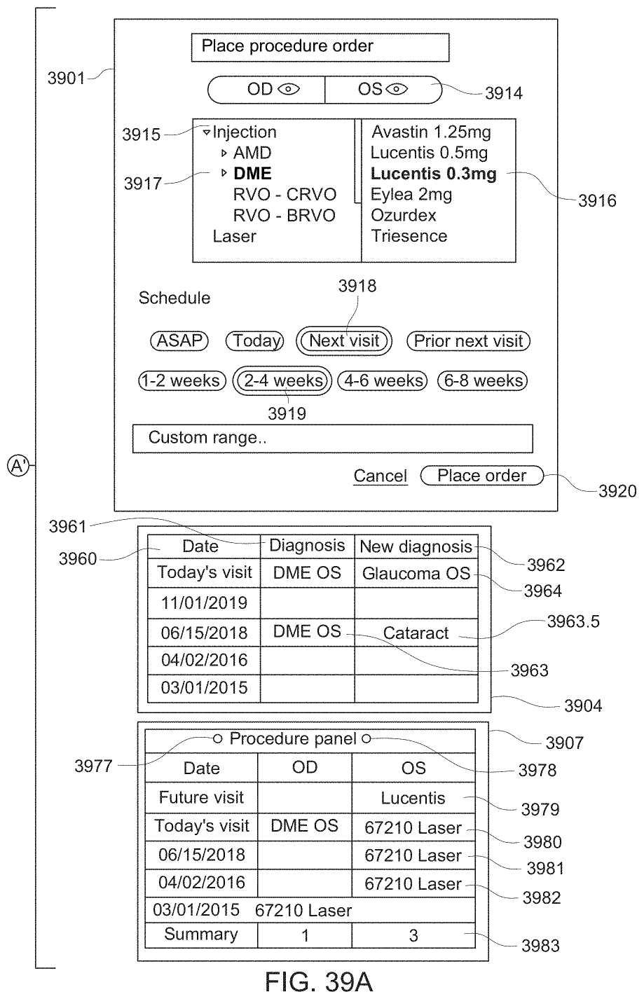

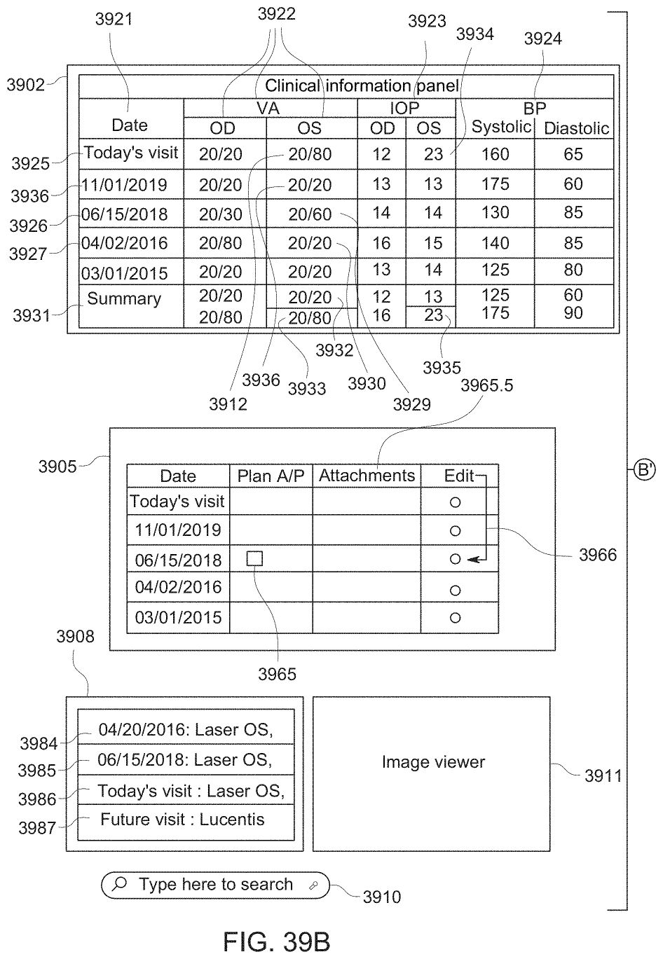

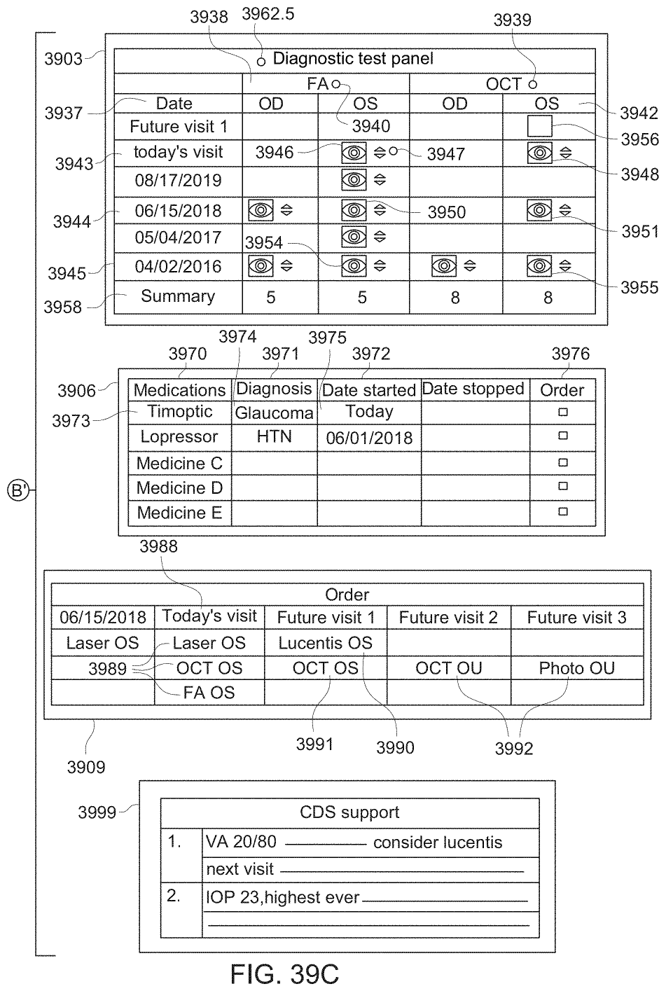

FIGS. 39A, 39B, and 39C depict an embodiment of a medical records dashboard in which a medical care provider is able to place orders in context, so as to enable the user to order as well as visualize orders placed, today or in the future with relevant data displayed. Clinical data, diagnostic tests and procedures are correlated, so at a glance doctors can determine what orders are needed, and then the orders are inserted in sequence, allowing for confirmation of the accuracy of the order placed. EMR companies have data dispersed throughout the EMR on separate windows. When EMR companies do present data on the same view, the information is difficult to compare and only are the same category of medical services correlated to each other. One embodiment of the invention compiles relevant data across categories of medical services as an order is placed on a dashboard in rows and columns. This new embodiment displayed in FIG. 39 allows for ready identification and tracking of relevant data of different medical services ie clinical data, examination data, procedures performed, diagnostic tests and even billing information, in separate modules or panels all uniquely correlated. This invention allows the options for multiple areas to be displayed with different data sets grouped into multiple panels or modules with related data generated and configured and highlighted to allow for efficient correlation by the user. Zooming, scrolling, and searching functions are enabled so user can see information that they want to see. Some embodiments where EMR systems require opening multiple windows or separate displays, the invention will highlight the relevant information no matter where the data is located.

FIG. 39 illustrates the modules all on one display with one user interface allowing the user a bird's eye view while highlighting relevant related information. Element module 3901 displays one means for an order for a medical service to be created by the doctor during today's visit or for a future visit. This ordering panel does not always have to be displayed. In fact, its contents and options for ordering change based on the context. There are many different ways to access and generate this overlay, element 3901. It can occur, for instance, by clicking on 3939 or, 3940 in element 3903, which is diagnostic testing module. So, accessing the ordering mechanism panel through particular panel specific data, would change the context for generating the overlay in panel 3901, and the information contained and input and output from element 3901 would be different. If instead of accessing 3901 from elements 3939 or 3940 within the diagnostic test module 3903 was instead generated from 3978 in element 3907, which is a panel displaying data for procedures, 3901 would generate an entirely different overlay or window with different information, rules, and analytics specific to ordering procedures. In fact, displayed in 3901 is an example of ordering mechanism for a particular type of procedure, an injection of a medication called Lucentis, 3916. Another example where the context of the ordering mechanism of 3901 would generate entirely different set of orders and rules is if 3976 were clicked in the module 3906 which is dedicated to data on medications.

Panels 3902-3906 can display relevant data related to the order being placed in 3901 so that doctors can make efficient and accurate medical decisions for a patient. In addition to displaying relevant data, the order created in 3901, can be displayed in the correct logical location, so the doctors can confirm the orders they are placing are correct. The orders can be displayed, in a variety of formats with panels generated to display the order with other relevant past, present or future orders such as seen in panels 3907, 3908 and 3909. Relevant data is displayed in 3902-3906 to allow for the orders to be placed in context with the doctor visualizing relevant data.

In FIGS. 39A, 39B, and 39C, element 3902 depicts where clinical data of any kind can be displayed with relevant data over a period of time. 3921 shows the time of the measurement of the clinical or examination data. 3922 illustrates a vision (VA) measurement with vision results separated into right (OD) and left (OS) eyes. 3923 is a pressure of the eye (IOP) column that can also be separated into right and left eye. 3924 is an example of another clinical measurement BP (blood pressure). Element 3903 shows a diagnostic testing panel, which can include any type of medical service most commonly represented by a CPT code. Displayed are examples of diagnostic tests. In the medical field examples include pathology results, chemistries, photographs, radiological procedures. The diagnostic tests displayed are 3938, an FA (Fluorescein Angiography) separated into OD (right) and OS (left). 3942 shows an example of a diagnostic test column of the left eye for a diagnostic test called OCT (Optical Coherence Tomography). By clicking any of the icons, for instance 3946 or 3950 the underlying data or images of diagnostic tests can be viewed, displayed and compared in the image viewer 3911. An interpretation of the test can be viewed or entered by the doctor by clicking on 3947. These icons, 3957, in addition to providing a mechanism to pull up directly the image or test, can demonstrate that a test was done, but also in limited space convey additional information such as a worsening highlighted for instance in red or improving test highlighted for instance in green. 3958 is a summary that can display the total number of a particular test performed in a patient. 3954 shows a total of 8 of test 3957 were performed over time for this patient. Displayed however is just one such test, 3957 because other tests are not relevant to issue being displayed to the doctor. However, the doctor can have access to the other 7 of 3957 by clicking in some embodiment on 3959.

An order for a particular diagnostic test today's or for a future visit can be ordered by any means, but displayed is one example by clicking 3940 or 3939 and either a means to order that test is accessed in 3901 or in 3903, through a mechanism described in FIG. 38, for instance, Element 3818. 3942.5 shows an area the doctor can click if they want to know financial information about any diagnostic test. A column next to each diagnostic test can be displayed which shows cost and payments which may help a doctor in deciding what to order in the future as perhaps some insurance will not pay for certain tests. 3904 shows a panel that displays patient's diagnosis or problem list or other data. 3960 is the date a diagnosis was recorded. 3961 is the diagnosis and 3962 is a column if the diagnosis is new and not recorded for this patient before.

3905 displays a panel that shows a means that a doctor's assessment and plans can be seen and directly accessed by clicking 3965. The plan can come up anywhere on the screen for instance over 3905 or 3911. All panels can be moved in some embodiments by the doctor. Doctor can view from 3905 any or all past or present plans and can also view attachments which may be scanned into the EMR, column 3965.5. The plans can be created, edited, or modified by, for instance, clicking 3966 and in some embodiments populated elsewhere into the chart 3906 shows a panel that displays the various medication a patient is on. 3970 is a column that displays a listing of the medications the patient is on. 3971 displays the diagnosis or condition that the medication is treating. 3972 displays the date the medication is started. Other columns can be added to display any information that is needed. Medications can also be ordered from this panel, with the ability to access an outside medication ordering platform. For instance, medications can be ordered by clicking in the columns 3976, which can activate a mechanism that generates an overlay to pull up software that can place medication orders. This invention allows these medication orders to be placed, even if connected from industry standard shared medication platforms. Unique to this invention, while a medication is ordered, relevant clinical, procedural or diagnostic data is visible to the user. This provides efficient, accurate medication orders to be placed. Bar graphs of the medications can also be displayed.

Window/panel 3907 displays relevant procedures performed on a patient. If finances are relevant, for instance, choosing a less expensive medication for an injection, clicking 3977 adds a column next to the procedure column showing financial information. Module 3911 shows a panel image viewer where diagnostic and images can be enlarged after directly accessing the data, by for instance, clicking on icons in the diagnostic Panel 3903 for example 3957. If the user wants more information shown in the panels with direct one click access or hovering the, underlying data and images can be displayed. For instance, clicking on 3946 or any of the diagnostic icons, allows for the actual study to come up and be evaluated in 3911, with relevant data displayed in all the panels to enhance interpretation. Panels that may not be needed when 3911 is displaying an image can collapse to allow for more room. For instance, collapsing 3908, 3905, 3910 and 3912 would allow 3911 to expand to enlarge the image associated with, for instance, 3946.

The panel 3912 displays a clinical decision support area where advice can be offered to the doctor. When a CDS message is offered to the doctor instead of just a pop up message as seen in some systems that offer advice or show a warning to a doctor, the system can display the relevant data that explains and supports CDS advice, so a doctor can quickly determine if the advice or warning is accurate. Similarly, just as when an order is placed, relevant data is displayed, so too the same display of relevant supporting data to the CDS can be displayed. Each module 3901-3909 and 3911 is a smart module. With each module able to display conclusions that help the doctor understand orders to place and visualize, the supporting data behind the CDS recommendations.

In element 3910, searches can be typed or through voice recognition a search of all the data in the invention, EMR and or PM system, the invention can display and correlate by highlighting information in each of the relevant panels, the answer that the search question asked. Suggestions of care known as clinical decision support, can be displayed in 3912.

Displayed in FIG. 39 is an example of a retina doctor determining how to treat a patient with a certain medical condition and then placing an order. In panel 3904, a patient is shown to have diabetic macular edema, DME 3963 in the left eye on Jun. 15, 2018 and Panel 3902 displays the same date also highlighted in blue in 3926. The vision (VA) is decreased to a level of 20/60, #3929. On that same day, Jun. 15, 2018, in module 3903, two diagnostic tests were performed, 3950 and 3951 also highlighted in blue. Panel 3907 and 3908 are two ways of demonstrating in #3981 and #3985 that on Jun. 15, 2018 a laser was performed also highlighted in blue. The doctor can then understand on that visit, what the patient's vision was, the diagnostic tests and that a laser was performed. The doctor also has displayed the immediate previous visit when the last laser in the left eye was performed, which the invention has determined is relevant to a laser being done on the same eye. The previous visit, where a laser was performed is yellow on 4/2/16 #3982. This is one of many methods the invention uses to differentiate all relevant information 6/15/18 laser date (blue) from the last laser date of Apr. 2, 2016 (yellow). Also highlighted in yellow are other medical services on 4/2/16, 3927, 3930, 3945, 3554, 3955, 3984. Element #3936 shows that on 11/1/19 the visit after 6/15/18 that patients vision improved from 20/60 (3929) to 20/20 (#3936), which is normal vision. The patient does not have any treatment such as laser on that day.

The patient, when they return on “today's visit,” all relevant data is displayed in each module. Once measurements on “today's visit” are entered, the invention can evaluate the data and if the rules engine, or through predictive analytics, or machine learning, suggestions for care or the modules detect data that needs to be brought to the doctor's attention, data can be highlighted and alerts created. Also, the CDS module can make suggestions and module 3912 displays, in FIG. 39, a message that says: “1. VA 20/80 worst is has ever been. Confirm with OCT (3948) that it is worsening therefore, consider laser now and Lucentis within one month. 2. IOP 23 today (3954), highest ever. Consider starting glaucoma medicine.” The invention allows the CDS message to display the evidence behind its suggestion by highlighting clinical findings shown in 3902 by any method ie. highlighting #3912, since the vision has greatly decreased to 20/80 and 3934 can also blink and be orange, alerting the doctor the IOP is also the worst at 23. In a summary row, the worst vision ever 3933, also is highlighted in orange and can blink, showing the doctors that 3912 is in fact the worst vision the patient ever had, and the best vision #3932 the patient has ever had is also shown on the summary row 3931. Displayed in 3902 is 3912 and 3933 are both orange, but any means can be used to illustrate to the doctor the connection of today's vision being the worst ever. This allows the doctor to quickly correlate what the vision was today compared to the past. Panel 3903 shows that today's visit ordered was a 3946 FA and a 3948 OCT. Element 3947 allows the doctor to either interpret or discover how good or bad that diagnostic test was and confirm the CDS claim of worsening. The doctor can see these clinical findings and the diagnostic findings and confirm that the CDS suggestion of a laser is correct.

In one example embodiment, a computer implemented method of creating medical orders includes generating a dashboard display comprising one or multiple visible panels having data corresponding to different respective medical services. A request to create an order is received in response to user interaction with a first one of the multiple panels. First medical information is retrieved as a function of information associated with the panel from which the request was received and a place order panel is populated with the retrieved first medical information. In one example, order information is received via the place order panel and second medical information is received in response to the order information. Selected multiple panels are then populated with the retrieved second medical information that is helpful in the ordering process. In one embodiment, the first and second medical information comprise multiple of clinical data, examination data, procedures performed, diagnostic tests and billing information provided in respective ones of the multiple visible panels. The first one of the multiple panels may comprise a procedure panel corresponding to a first procedure, and the second medical information may include diagnostic information correlated to the first procedure for display in a diagnostic panel and prior procedures performed correlated to the first procedure for display in the procedure panel. The second medical information may include examination data correlated to the first procedure for display in a clinical information panel. The second medical information may include medication data correlated to the first procedure for display in a medication information panel. In one embodiment, the second medical information that is displayed in the respective panels is highlighted. The first medical information may include data covering a selected period of time such as past, present and future times. The method may also include receiving user input corresponding to a diagnostic test icon in a diagnostic panel, retrieving an image corresponding to the diagnostic test, and displaying the image in an image viewer panel. In one embodiment, the first one of the multiple panels comprises a medication panel, and wherein selection of an order icon from the medication panel generates an overlay for ordering medications. in a further embodiment, one of the multiple panels comprises a clinical decision support panel that includes clinical decision support information that includes supporting data. One of the multiple panels comprises a diagnostic panel populated with retrieved first medical information comprising historical diagnoses data.

The doctor then on 3901 places a procedural order. One way of ordering a procedure is by clicking #3978, which is within a module that generate and displays relevant procedures. The tool understanding the context is accessing the procedure panel from 3907 to order a procedure such as a laser, this generates an overlay layer or window seen in 3901 with information that receives input to allow the doctor to place an order for a procedure, but also as the doctor places the order, so a confirmation of the proper order can be visualized. The user visualizes that the order they are placing for a laser is a repeat of a previous laser because when the laser is populated in “todays visit” in Element #3980 it is lined up with the other lasers performed in the left eye in the past 3981, 3982. The doctor can see this is a repeat laser and can visualize that this and is the third laser that is going to be done in this left eye. The summary row number 3 is displayed 3983 and confirms that fact and the 3 is also orange confirming the laser today 3980 is the third time the laser has been done in the left eye. Panel 3908 also displays #3986 a laser is to be done today. Panel 3909 shows that today's visit, #3988 that a laser is to be performed as well as an OCT and FA 3989. The FA and OCT are also displayed in panel 3903 as 3946 and 3948. The doctor has now determined what action to take and what to do today, i.e., order, a laser to be done today and can visualize it is being properly ordered and to be billed in the left eye today with all of the diagnostic tests 3946 and 3948. Sometimes an accidental click can cause the order to be placed in the wrong part of the body but by visualizing the order in context with the past, the doctor can confirm that the order is in the correct part and side of the body. In this case, the left not the right eye and can confirm the order by clicking for instance, #3920. Now, the doctor turns their attention to the future visit, and what they plan to do in the future. In this particular example CDS suggested a Lucentis injection was needed in the future. The doctor is shown since the patient's vision today was the worst it has ever been, in #3912, and the diagnostic test performed 3946 and 3948 the doctor can confirm the need for this Lucentis by displaying the image in 3911, the doctor can confirm, with all of this relevant information present, to now on the next visit, injection in the eye with a medication that can help the retina improve and by performing both the laser “today” displayed in 3980 as well as in a future visit an injection of medication in the eye, displayed as Lucentis as, this may be the best medication for the patient, which CDS suggested. Once again, the doctor turns to procedural panel 3901 by clicking 3978 or activating 3901 by any other means and places an order. Displayed, is the doctor choosing on 3914 the left eye. 3915 chooses an injection. 3916, the injection that the doctor selects is a medication called Lucentis. 3917 shows the doctor explaining why they are doing the injection by selecting a ICD-10 diagnostic code illustrated as DME, which stands for diabetic macular edema, also displayed in 3963. The tool knows the reason and could automatically populate the reason or diagnosis. 3918 shows the doctor wants to inject Lucentis at the next visit. 3919 shows that the next visit the doctor orders to be in two to four weeks. Now in panel 3907, 3979 shows the future visit, and that the doctor is ordering Lucentis and populates that cell. Now the doctor can see, in context, what this future visit looks like and that the previous procedures in 3980 (todays visit) 3981 6/15/18 visit and 3982 4/2/16 visit performed in the left eye and the doctor can re-confirm that this is exactly what they want to do. #3987 is another mechanism to show the future visit of Lucentis displayed. Now with the doctor seeing this and also being able to have direct access to any of the diagnostic tests displayed in 3903 and could, for instance, look at 3946 or 3948 to confirm the Lucentis injection is needed. Once the decision is clear, and what they are ordering is proper, they can then click 3920 confirming the order.

The doctor may decide that another diagnostic test should be performed on this future visit, so clicking 3939, which is the OCT column or going back to 3901 and choosing, instead of procedures, the diagnostic tests, can order an OCT for the next visit. That OCT or whatever diagnostic test that the doctor might want to order, the same methods described for ordering the Lucentis injection displayed in 3901 can be repeated to order an OCT in the left eye on the future visit. Once ordered the OCT is displayed in 3956 future visit, as well as 3989, which would become a permanent order once 3920 is clicked. It is a square, empty box 3956 because the OCT has not yet been performed. Whereas in 3948, it is a filled in square or icon because the OCT has been performed. There is also a new diagnosis seen, glaucoma, on 3964 which explains why a new medication also in blue was started that day in 3906, Timoptic 3973. CDS suggested that IOP was the highest ever at 23 and 3934 is orange and 3535 is orange alerting the doctor this is the highest pressure ever. Now the doctor if they agree with the CDS can click on 3976 and the process is repeated and they order a glaucoma drug 3973 and 3975 shows the medicine starting today.

Through this mechanism, the doctor can place orders, see all relevant information and make certain their orders are correct and confirm if the CDS messages and alerts are accurate at a glance seeing the data that supports the recommendations. Finally the orders placed appear in the panels in row displaying today's visit. For example 3943, 3980 and the future visit, for example 3956, 3979, and the doctor can visualize the orders that they placed are exactly as they meant to order in the correct part of the body in the time period the doctor wants and by clicking 3920 confirms the orders.

For example, in some embodiments in accordance with the present principles, Rules and Configurations can be predetermined and stored, for example, in the Rules module 004, for determining which data of a Flowsheet and, as such, which portions of the medical records dashboard to display or hide. Alternatively or in addition, in some embodiments, a user can self-configure the medical records dashboard to display only certain portions or to hide certain data of a Flowsheet and, as such, which portions of the medical records dashboard to display or to hide using, for example, a user interface (not shown) associated with the medical records dashboard. Alternatively or in addition, data of the Flowsheet can contain an indicator (e.g., a flag) that can be identified by, for example, the Rules module 004, for determining when and if a piece of data should be displayed or hidden.

In some embodiments, the Data Command center 001 enables the medical records dashboard to intelligently expand, collapse, display, and/or hide columns, rows and/or any other portion of the medical records dashboard to show precisely what a user wishes to display. For example, in one embodiment, a Flowsheet including patient treatment and health information can be accessed from an EHR system using, in some embodiments, an icon/button, keystroke, or series of keystrokes associated with at least one of the Data Command center 001 and the medical records dashboard. Upon accessing the Flowsheet, a set of Rules and Configurations associated with, for example, the Rules module 004 of the Data Command center 001, can be evaluated to determine which data from the Flowsheet is to be displayed in the medical records dashboard. For example, in some embodiments, the Rules module 004 can include information on what data to display, and in turn what portions of the medical records dashboard to display, based on, including but not limited to, at least one of an identity of a medical care provider, an identity of a patient, a medical care provider's specialty, conditions of a patient, patient procedures, risk factors, diagnostic results, future orders, future appointments, values recorded, values not recorded, calculated values, and absolute values for display.

For example, in some embodiments in accordance with the present principles, Rules and Configurations can be predetermined and stored, for example, in the Rules module 004, for determining which data of a Flowsheet and, as such, which portions of the medical records dashboard to display or hide. Alternatively or in addition, in some embodiments, a user can self-configure the medical records dashboard to display only certain portions or to hide certain data of a Flowsheet and, as such, which portions of the medical records dashboard to display or to hide using, for example, a user interface (not shown) associated with the medical records dashboard. Alternatively or in addition, data of the Flowsheet can contain an indicator (e.g., a flag) that can be identified by, for example, the Rules module 004, for determining when and if a piece of data should be displayed or hidden.

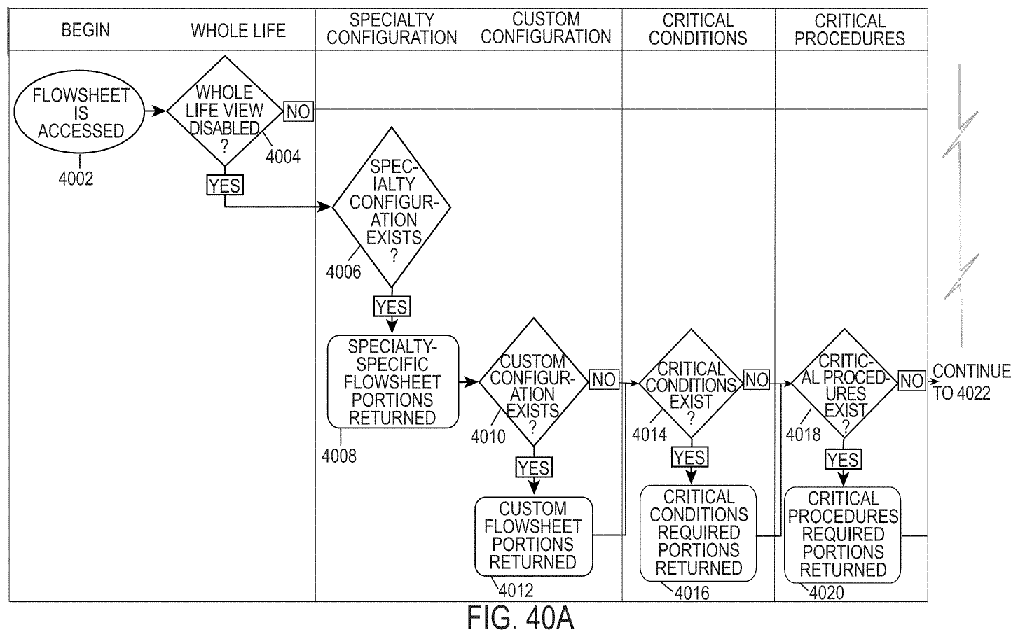

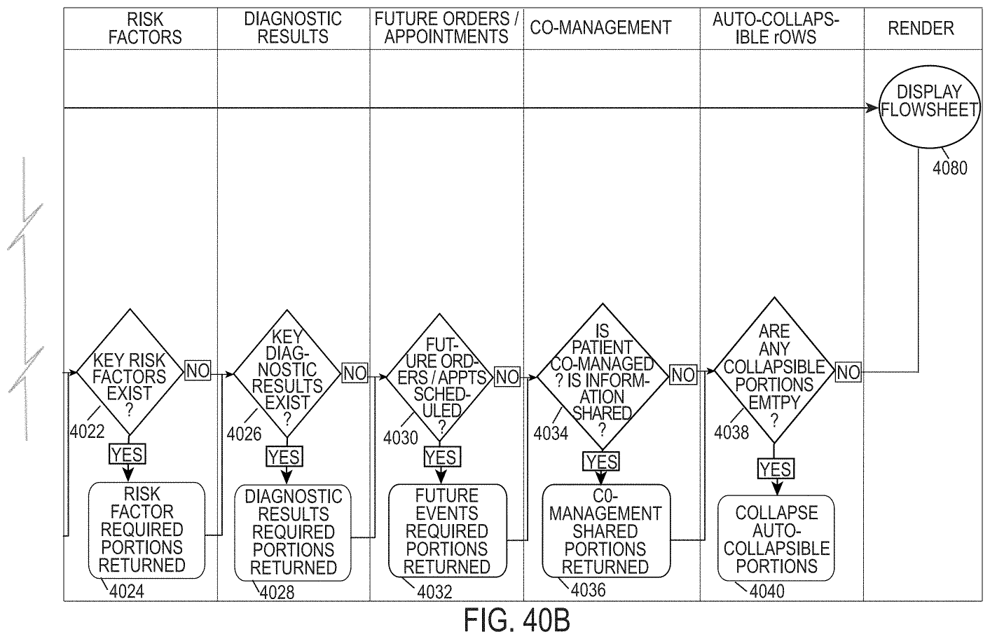

FIGS. 40A and 40B (referred to collectively herein as FIG. 40) depict a workflow diagram of a process for intelligently expanding, collapsing, displaying, and/or hiding columns, rows and/or any other portion of the medical records dashboard in accordance with an embodiment of the present principles. In the embodiment depicted in FIG. 40 the process begins at 4002 during which a Flowsheet including patient treatment and health information is accessed from, for example, an EHR system. The process illustratively proceeds to 4004. At 4004, it is determined if, what the inventors refer to as a “Whole Life View”, is disabled. More specifically, At 4004 it is determined if all the data in the Flowsheet should be displayed in the medical records dashboard. If Whole Life View is disabled, the process proceeds to 4080 during which all of the data from the Flowsheet is displayed in the medical records dashboard. If not, the process illustratively proceeds to 4006.

At 4006, it is determined if at least one Specialty Configuration exists. For example, in some embodiments a Specialty Configuration can include a configuration based on the specialty of a medical care provider. If so, the process proceeds to 4008 during which all Specialty Configurations are identified such that the data from the Flowsheet can be filtered to only display data associated with identified Specialty Configurations. For example, as previously described, in some embodiments information associated with medical care provider specialties and data to be displayed and hidden in the medical records dashboard dependent on the specialties can be predetermined and stored in the Rules module 004. In accordance with the present principles, Specialty Configurations can require certain portions, columns, and/or rows of the medical records dashboard to be displayed or hidden. After the Specialty Configurations are identified and/or if it is determined that a Specialty Configuration does not exist, the process illustratively proceeds to 4010. In accordance with the present principles, data from the Flowsheet to be displayed in or hidden from the medical records dashboard can be filtered using the identified Specialty Configurations.

At 4010, it is determined if at least one Custom Configuration exists. If so, the process proceeds to 4012 during which all Custom Configurations are identified such that the data from the Flowsheet is filtered to only display data or hide data associated with the identified Custom Configurations. For example, in some embodiments custom configurations and data to be displayed in or hidden from the medical records dashboard dependent on the custom configurations can be predetermined and stored in the Rules module 004. Alternatively or in addition, in some embodiments, a user can use a user interface associated with the medical records dashboard to create and/or identify custom configurations. In accordance with the present principles, Custom Configurations can require certain portions, columns, and/or rows of the medical records dashboard to be displayed or hidden. After the Custom Configurations are identified and/or if it is determined that a Custom Configuration does not exist, the process illustratively proceeds to 4014. In accordance with the present principles, data from the Flowsheet to be displayed in or hidden from the medical records dashboard can be filtered using the identified Custom Configurations.

At 4014, it is determined if at least one Critical Condition exists. That is, in some embodiments, critical conditions can be identified that, no matter what rules indicate that certain data should not be displayed or hidden, the identified critical conditions are to be displayed in at least one location of the medical records dashboard 400. In some embodiments, Critical Conditions can be identified and stored in the Rules module 004. Alternatively or in addition, a user can identify Critical Conditions using a user interface associated with the medical records dashboard 400. If it is determined that at least one Critical Condition exists, the process proceeds to 4016 during which the Critical Conditions are identified such that any data from the Flowsheet identified as a Critical Condition can be displayed in at least one portion of the medical records dashboard 400. In accordance with the present principles, Critical Conditions can require certain portions, columns, and/or rows of the medical records dashboard to be displayed or hidden. After the Critical Conditions are identified or if it is determined that a Critical Condition does not exist, the process illustratively proceeds to 4018.

At 4018, it is determined if at least one Critical Procedure exists. That is, in some embodiments, critical procedures can be identified that, no matter what rules indicate that certain data should not be displayed or hidden, data associated with the identified critical procedures are to be displayed in at least one location of the medical records dashboard 400. In some embodiments, Critical Procedures can be identified and stored in the Rules module 004. Alternatively or in addition, a user can identify Critical Procedures using a user interface associated with the medical records dashboard 400. If it is determined that at least one Critical Procedure exists, the process proceeds to 4020 during which data associated the Critical Procedures are identified such that any data from the Flowsheet identified as being associated with a Critical Procedure can be displayed in at least one portion of the medical records dashboard 400. In accordance with the present principles, Critical Procedures can require certain portions, columns, and/or rows of the medical records dashboard to be displayed or hidden. After the Critical Procedures are identified or if it is determined that a Critical Procedure does not exist, the process illustratively proceeds to 4022.

At 4022, it is determined if at least one Risk Factor exists. That is, in some embodiments, Risk Factors can be identified that, no matter what rules indicate that certain data should not be displayed or hidden, the identified Risk Factors are to be displayed in at least one location of the medical records dashboard 400. In accordance with the present principles, Risk Factors can require certain portions, columns, and/or rows of the medical records dashboard to be displayed or hidden. For example, a smoker with high blood pressure, and diabetes having an identified Risk Factor for a heart attack can require a visual field column with an alert to be displayed in at least a portion of the medical records dashboard 400. In some embodiments, Risk Factors can be identified and stored in the Rules module 004. Alternatively or in addition, a user can identify Risk Factors using a user interface associated with the medical records dashboard 400. If it is determined that at least one Risk Factor exists, the process proceeds to 4024 during which the Risk Factors are identified such that any data from the Flowsheet identified as identifying a Risk Factor can be displayed in at least one portion of the medical records dashboard 400. After the Risk Factors are identified or if it is determined that a Risk Factor does not exist, the process illustratively proceeds to 4026.

At 4026, it is determined if at least one Key Diagnostic Result exists. That is, in some embodiments, Diagnostic Results that are considered Key can be identified that, no matter what rules indicate that certain data should not be displayed or should be hidden, data associated with the identified Key Diagnostic Results are to be displayed in at least one location of the medical records dashboard 400. In accordance with the present principles, Key Diagnostic Results can require certain portions, columns, and/or rows of the medical records dashboard to be displayed or hidden. For example, if a lab returns a positive infectious disease test, data associated with that Key Diagnostic Result can be caused to be displayed in at least a portion of the medical records dashboard 400. In some embodiments, Key Diagnostic Results can be identified and stored in the Rules module 004. Alternatively or in addition, a user can identify Key Diagnostic Results using a user interface associated with the medical records dashboard 400. If it is determined that at least one Key Diagnostic Results exists, the process proceeds to 4028 during which the Key Diagnostic Results are identified such that any data from the Flowsheet identified as being associated with a Key Diagnostic Results can be displayed in at least one portion of the medical records dashboard 400. After the Key Diagnostic Results are identified or if it is determined that a Key Diagnostic Results does not exist, the process illustratively proceeds to 4030.

At 4030 of the embodiment of FIG. 40, it is determined if at least one Future Order/Appointment exists. That is, in some embodiments, Future Orders/Appointments can be identified that, no matter what rules indicate that certain data should not be displayed or should be hidden, data associated with the identified Future Order/Appointment are to be displayed in at least one location of the medical records dashboard 400. In accordance with the present principles, Future Orders/Appointments can require certain portions, columns, and/or rows of the medical records dashboard to be displayed or hidden. For example, if an Open-heart surgery is scheduled for the future, it can be desirable for all medical care providers to see the scheduled Open-heart surgery in at least a portion of the medical records dashboard regardless of a medical care provider's specialty. In some embodiments, Future Orders/Appointments can be identified and stored in the Rules module 004. Alternatively or in addition, a user can identify Future Orders/Appointments using a user interface associated with the medical records dashboard 400. If it is determined that at least one Future Order/Appointment exists, the process proceeds to 4032 during which the Future Orders/Appointments are identified such that any data from the Flowsheet identified as being associated with a Future Order/Appointment can be displayed in at least one portion of the medical records dashboard 400. After the Future Orders/Appointments are identified or if it is determined that a Future Order/Appointment does not exist, the process illustratively proceeds to 4034.

At 4034, it is determined if Co-Management of at least one patient is allowed and if patient information sharing is allowed. That is, in some embodiments, Co-Management of patients can require certain portions, columns, and/or rows of the medical records dashboard to be shared or hidden amongst different users/medical care providers. For example, if a medical records dashboard in accordance with the present principles is being used by multiple medical care providers to care for a patient, the patient's primary care physician is able to see lab results from a specialist if the specialist has shared at least the relevant portions of a medical records dashboard. In some embodiments, patient data/information to be shared and, as such, portions of a medical records dashboard to be shared can be identified and stored in the Rules module 004. Alternatively or in addition, a user can identify patient data/information to be shared and, as such, portions of a medical records dashboard to be shared using a user interface associated with the medical records dashboard. If it is determined that Co-Management of at least one patient exists and if patient information sharing is allowed, the process proceeds to 4036 during which the existence of Co-Management of at least one patient and patient information sharing is identified such that any data from the Flowsheet identified as being associated with Co-Management and patient information sharing can be displayed in at least one portion of the medical records dashboard 400. After the Co-Management and patient information sharing is identified or if it is determined that Co-Management and patient information sharing does not exist, the process illustratively proceeds to 4038.

In the embodiment of FIG. 40, at 4038, it is determined if any of the collapsible portions, columns, and/or rows of the medical records dashboard contain no respective values (i.e., are empty). If it is determined that collapsible portions, columns, and/or rows of the medical records dashboard contain no respective values, the process proceeds to 4040 during which the collapsible portions, columns, and/or rows of the medical records dashboard 400 containing no respective values can be collapsed or hidden from display on a least a portion of the medical records dashboard. After all of the display configurations have been determined as described above, at 4080 the data of the Flowsheet to be displayed, as determined by the process of FIG. 40 described above, is displayed in the medical records dashboard 400. The process can then be exited.

In accordance with the present principles and as described above, in some embodiments, rules determine portions, columns, and/or rows of the medical records dashboard to expand or display based on predefined criteria, and also determine portions, columns, and/or rows of the medical records dashboard to collapse or hide based on the predefined criteria, and can also determine portions, columns, and/or rows of the medical records dashboard to flag or highlight based on the predefined criteria. For example, in some embodiments, the entirety of a patient's accessible records can be viewed. In some embodiments, the entirety of a patient's accessible records are evaluated against specialty and user-specific configuration criteria (e.g., Rules), actively collapsing or hiding portions, columns, and/or rows of the medical records dashboard deemed unnecessary for a user or specialty and actively enabling the display of portions, columns, and/or rows of the medical records dashboard deemed relevant to the user or specialty. In some embodiments, an intelligent Rules system actively determines which portions, columns, and/or rows of the medical records dashboard to display based on a user, a user's specialty, a patient, a patient conditions, a patient procedures, risk factors, diagnostic results, future orders, future appointments, values recorded, values not recorded, calculated values, and absolute values for display. In another embodiment, shared portions, columns, and/or rows of the medical records dashboard between medical care providers and facilities can be added or expanded based on preconfigured or point-of-sharing decisions made by the sharing medical care providers.

Although the embodiment of the process for intelligently expanding, collapsing, displaying, and/or hiding columns, rows and/or any other portion of the medical records dashboard of the present principles described with reference to FIG. 40 illustratively comprises specific Rules-based configurations, other embodiments of the process in accordance with the present principles can comprise any combination of some or all of the described Rules-based configurations and can also comprise other Rules-based configurations. Even further, those skilled in the art will appreciate that the order of operations denoted in the process above with reference to FIG. 40 can be non-linear and optimized based on usage and workflow. That is, order, inclusion, and omission can be intelligently determined based on accessibility of data, predefined configurations, real-time user selection, custom configurations, preferred practice patterns, and/or workflow.

In addition, although in the embodiment of the process for intelligently expanding, collapsing, displaying, and/or hiding columns, rows and/or any other portion of the medical records dashboard of the present principles described with reference to FIG. 40 the Rules are described as being stored in the Rules module 004, those skilled in the art will appreciate that rules and configurations of a process of the present principles can be stored in tables, accessed remotely via API or other digital communications technology, or generated on-the-fly as the result of calculations during the operations. Rules and configurations can be stored within the application or reference outside data sources. Rules and configurations can be altered by the user, in some embodiments, by the application, in some embodiments, and/or by outside resources.

In addition, although in the embodiment of the process for intelligently expanding, collapsing, displaying, and/or hiding columns, rows and/or any other portion of the medical records dashboard of the present principles described with reference to FIG. 40 it is described that upon rendering the Flowsheet, data populates within the columns specified, in some embodiments, further rules and configurations can apply post-rendering, based on data returned and/or calculated within columns. In addition, in some embodiments, manual manipulation allows for human interaction with the finally determined dataset. As such, a user can acknowledge and remove portions, columns, and/or rows of the medical records dashboard once they have been rendered. Removal of such portions, columns, and/or rows of the medical records dashboard can be one-time, or permanent unless a subsequent event retriggers the rendering of those portions, columns, and/or rows of the medical records dashboard, and such rendering can be patient-specific, provider-specific, location-specific, or otherwise tied to an event, condition, or trigger.

In one example of the process of the present principles, a dentist can access a Flowsheet for a patient with a rare blood disorder. As a dentist, the returned set of data to be displayed in accordance with a process of the present principles would ordinarily include data germane to dentistry, collapsing or hiding certain portions, columns, and/or rows of the medical records dashboard with no values present and/or deemed unnecessary. The dentist can have also chosen not to view certain portions, columns, and/or rows of the medical records dashboard as a matter of practice. In accordance with embodiments of the present principles, as a patient with a rare blood disorder, additional portions, columns, and/or rows of the medical records dashboard could be added to the display to reflect the patient's condition of the rare blood disorder and such information could be highlighted/flagged to alert a user as to the importance of the information being displayed.

In another example, an ophthalmologist sees a diabetic patient with no diagnostic testing for a chronic illness. As an ophthalmologist, the patient data ordinarily returned for display by a process of the present principles would ordinarily include data germane to ophthalmology, collapsing or hiding certain portions, columns, and/or rows of the medical records dashboard with no values present or data deemed unnecessary for display by the process. In some embodiments, the ophthalmologist can have also chosen not to view certain columns as a matter of practice. As a patient with a lapse in testing and underlying condition requiring testing, portions, columns, and/or rows of the medical records dashboard having no value present which would normally be collapsed/hidden, could now be expanded/displayed, and highlighted or flagged to draw the attention of a user to the lack of testing having been performed on the patient.

In a third example, a primary care physician (PCP) may wish to view an entire patient history. The patient history can consist of patient care provided by the PCP, patient care provided by doctors in the same office as the PCP, and patient care provided by specialists outside the practice that co-manage the patient and have shared data with the PCP. In this arrangement, the entire dataset is provided for viewing on the medical records dashboard for care provided by the PCP and doctors within the same practice, and a shared dataset can be provided for viewing on the medical records dashboard for care provided by the specialists. Columns with no values can be collapsed or hidden if no value exists as described above.

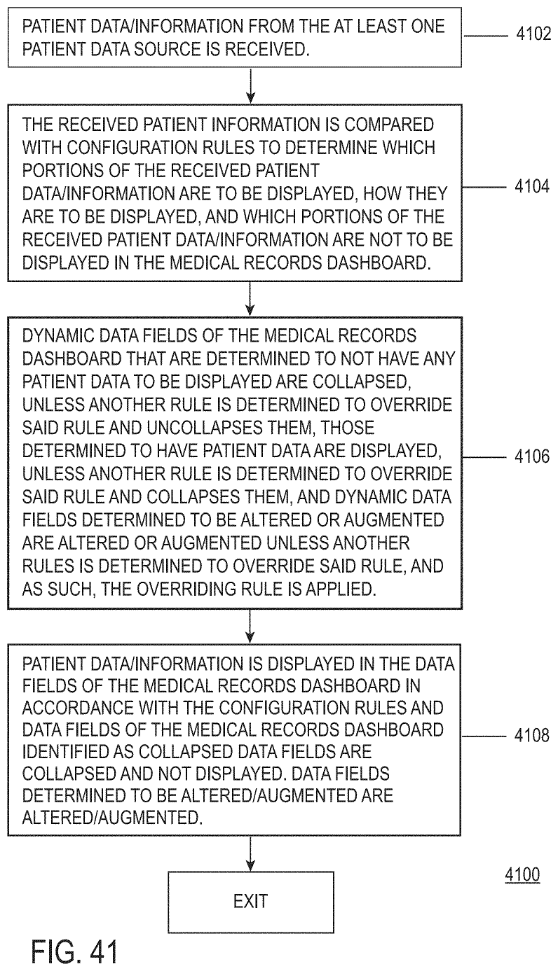

FIG. 41 depicts a flow diagram 4100 of a method for rules-based data display in a data command center comprising a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of collapsible data entry fields for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method beginning at 4102 during which patient data/information from the at least one patient database is received. The method 4100 can proceed to 4104.

At 4104, the received patient information is compared with configuration rules to determine which portions of the received patient data/information are to be displayed and which portions of the received patient data/information is not to be displayed in the medical records dashboard. The method 4100 can proceed to 4106.

At 4106, collapsible data entry fields of the medical records dashboard that are determined to not have any patient data to display are identified as collapsed data entry fields. The method 4100 can proceed to 4108.

At 4108, patient data/information is displayed in the data entry fields of the medical records dashboard in accordance with the configuration rules and data entry fields of the medical records dashboard identified as collapsed data entry fields are collapsed and not displayed. The method 4100 can then be exited.

In some embodiments the collapsible data entry fields identified as collapsed data entry fields comprise at least one of a column and a row of the medical records dashboard.

In some embodiments, the Data Command Center of the present principles, such as the Data Command center 001 of FIG. 1, provides a user(s) with the ability to collate data and visualize the correlation between different, related datapoints, each with their own distinct visualizations (considered by the inventors as a Correlative Line Graph display). Novel to customizable visualizations is to display an array of customized visualizations correlated on a comparative axis or axes. In some embodiments of the preset principles, the customized, correlative display consists of one or more visualizations of patient data and other data related to the Data Command Center data, horizontally, vertically, on a Z axis, or on multiple axes displaying multiple events, results, and/or calculations. In some embodiments, the Customizable, Correlative Line Graph display can be launched from within a medical records dashboard of the present principles using an icon/button, keystroke, or series of keystrokes.

Upon launch, the Customizable, Correlative Line Graph can display as a pop-up window, popover window, pop-out window, or other display format that enables the simultaneous accessibility of the Correlative Line Graph and the medical records dashboard of the present principles. The Graph may overlay or adjoin an underlying medical records dashboard in opaque or transparent states, be pinned to the medical records dashboard, and/or may hover over or aside the medical records dashboard.

Upon initiating the Customizable, Correlative Graph, a series of actions are performed to determine data and format of data displayed. Preconfigured CCG displays may be stored in tables or generated on-the-fly based on key considerations such as those laid out in Collapsible Columns and Rows, and those laid out in Guiding Actions in a Dashboard.

In one embodiment, relevant data is visualized graphically, as a series of events graphed against a timeline, correlated with a series of results, a series of actions, and a series of contributing factors. Any number of relevant details may be correlated as needed.

Data visualization is achieved with a series of configurations to determine what and how to display. In one embodiment, Source Data consists of a Value, an Inclusion/Exclusion Rule, and a Visual Representation Configuration. The data may consist of one type, a series of data points collected, values captured, validated for inclusion, and visualized across 2 intervals, correlated against a second type, a series of separate data points collected, values captured, validated for inclusion, and visualized across the same 2 intervals, correlated with a third type, a series of data points collected, values captured, validated for inclusion, and visualized across the same 2 intervals.

Rendered Customizable, Correlative Graphs may be interacted with in such ways as to turn on or off represented values in a similar manner to manually expanding/collapsing of columns and rows, i.e. turning on or off subsections of data, individual visualizations categorized by rows or columns, or selecting key elements to only display, selecting key icons within the display, and/or moving elements between positions to achieve a different view.

Those skilled in the art will appreciate additional visualizations may be added, additional flags derived, and a series of rules explained through this patent to manifest in the final rendering. Those skilled in the art will appreciate that the above described algorithm may be non-linear, may be automated in whole or in individual or groups of steps, and algorithms may intelligently update, flag, or otherwise override certain steps of the rendering process. Those skilled in the art will appreciate that single axis representation in the above description does not preclude multi-dimensional representations with multiple parallel representations as well as multiple perpendicular, or otherwise non-parallel representations.

The Customizable, Correlative Graph reaches its logical end at which point all data is rendered, processing of rendered data has occurred, and any/all necessary actions have been taken based on the processed data, including, but not limited to, Flags, Alerts, Clinical Decision Support, and Auto-Tasks. Auto-updates to patient data may initiate refactoring of the Customizable, Correlative Graph.

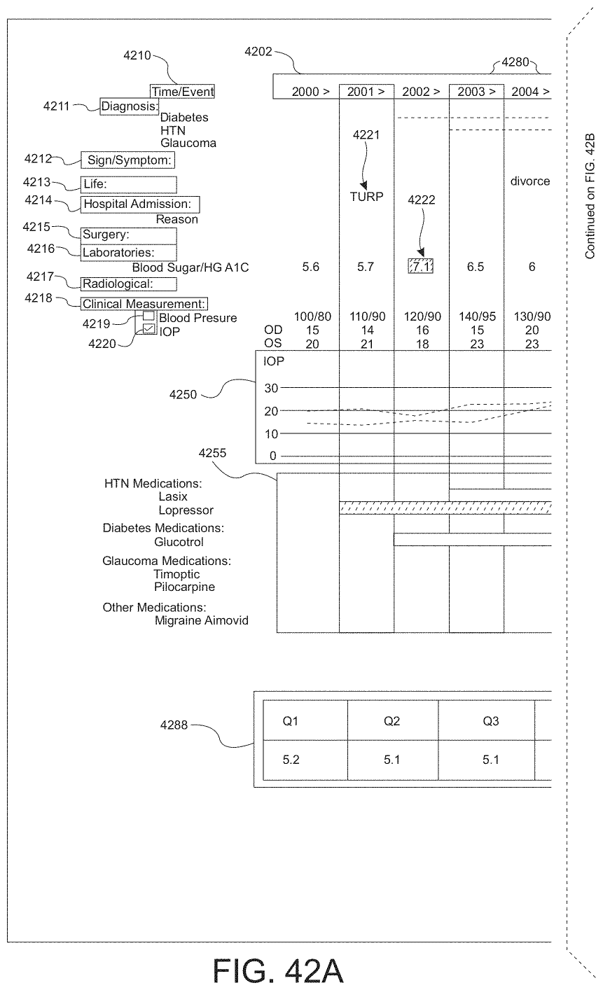

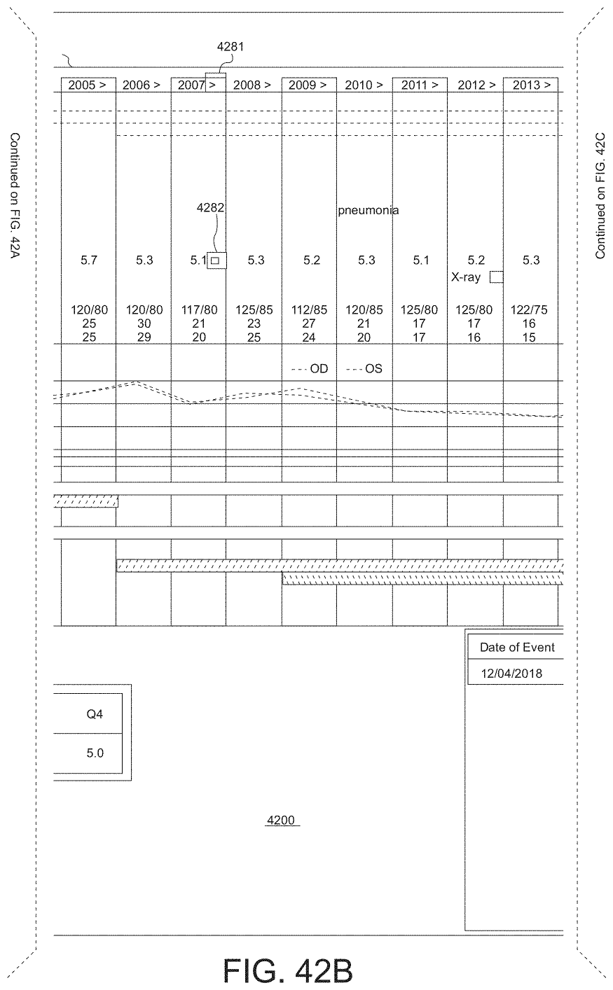

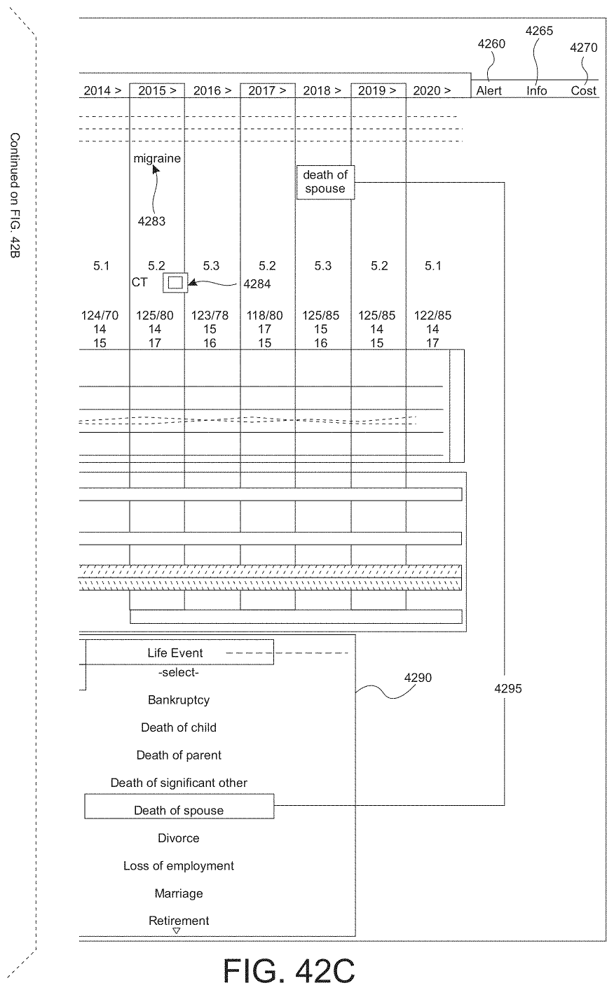

In some embodiments, the Data Command Center of the present principles, such as the Data Command center 001 of FIG. 1 enables, either as part of a medical records dashboard of the present principles or individually as a Whole Life tool, a user/medical care provider to graphically view, in a single display, a patient's entire medical history. For example, FIG. 42A, 42b, AND 42C depict a graphical view of the entire medical history of a patient as a Whole Life tool in accordance with an embodiment of the present principles. In the embodiment of FIG. 42A, 42b, AND 42C, the Whole Life tool 4200 illustratively lists dates, in one year incremented columns, across a top row 4202 of the Whole Life tool for a period of 20 years from 2000 through 2020. Although in the embodiment of FIG. 42A, 42b, AND 42C the time increments are illustratively one year increments, in other embodiments the time increments can be substantially any time increments chosen by the user/medical care provider.

In the embodiment of FIG. 42A, 42b, AND 42C, the Whole Life tool 4200 in a first column 4210 lists a series of life events that occurred in a patient's life including diagnosis 4211 given to the patient, signs and symptoms 4212 the patient has had, major life events 4213 of the patient, hospital admissions 4214, surgeries 4215 the patient has had, laboratories 4216 performed on the patient, radiological procedures 4217 performed on the patient, and clinical measurements 4218 made on the patient. The Whole Life tool 4200 of FIG. 42A, 42b, AND 42C further illustratively includes an IOP section 4250 graphically displaying the intraocular pressure of a patient's right eye (OD) and the patient's left eye (OS) as a line graph spanning the 20 depicted years of the patient's medical history. In the embodiment of FIG. 42A, 42b, AND 42C, the line graph of the IOP of a patient's right eye (OD) is color-coded red and the line graph of the IOP of the patient's left eye is color-coded blue for easier distinction. In the embodiment of the Whole Life tool 4200 of FIG. 42A, 42b, AND 42C, a lower section 4260 graphically displays a medication history for the patient. In FIG. 42A, 42b, AND 42C, horizontal bar graphs depict a history of the medication taken by and/or prescribed to a patient spanning the 20 depicted years of the patient's medical history. In the embodiment of FIG. 42A, 42b, AND 42C, the various medication bar graphs can be color-coded to more easily distinguish between medications. In some embodiments, color standards, such as defined by the American Academy of Ophthalmology, can be used for color coding the medications. Alternatively or in addition, in some embodiment custom colors can be used.

In the Whole Life tool 4200 of FIG. 42A, 42b, AND 42C any column, 4281, can be selected 4280 and expanded to take up the entire page, or a partial part of the page, or a navigation template 4290 may be used to navigate the timeline by date range or to zoom in on specific results for that time increment 4282. For example, if a user/medical provider selects the year 2007, that particular year can expand so that instead of displaying one full year as depicted in FIG. 42A, 42b, AND 42C, the Whole Life tool 4200 can display 12 months in the year in one month increments or quarterly or in any other increments, for example, for every medical encounter the patient has had. In some embodiments, a user/medical care provider is enabled to select whether to display all the encounters that the patient has had with any medical care providers or just particular medical care providers. In some embodiments, a zoom view of a particular time span can be displayed on another monitor such that a user/medical care provider is able to view the zoomed time increment simultaneously with the whole life view.

In the embodiment of the Whole Life tool 4200 of FIG. 42A, 42b, AND 42C, the patient illustratively had three major disease states, diabetes, hypertension, and glaucoma, as listed in the diagnosis row 4211. The Whole Life tool 4200 enables a user/medical care provider to select any of the identified major disease states to find out more detailed data regarding the selected disease state and update start/stop dates or activate/inactive a diagnosis. As depicted in FIG. 42A, 42b, AND 42C, a user/medical care provider is able to determine when the disease exactly occurred by referring to the Whole Life tool 4200. In the embodiment of FIG. 42A, 42b, AND 42C, the diabetes occurred in 2002, 2003 was hypertension, and 2006 was glaucoma. These are chronic diseases, and these are the dates of onset. In some embodiments, the Whole Life tool can include a bar graph that can continue along a horizontal date line displaying the time period that the patient had that diagnosis, and if for some reason they no longer had that diagnosis, the bar graph could stop.

The Whole Life tool 4200 of FIG. 42A, 42b, AND 42C displays for a user/medical care provider in row 4212 when a patient developed a symptom and identify the symptom 4283. Similarly, the Whole Life tool 4200 of FIG. 42A, 42b, AND 42C is able to display for a user/medical care provider in row 4213 when a major life event that can affect the well-being of a patient occurred such as a divorce or the loss of a loved one, etc. As previously described, in row 4214, the Life tool 4200 of FIG. 42A, 42b, AND 42C is able to display for a user/medical care provider hospital admissions the patient had over the 20 years spanning the patient's recorded medical history. In the embodiment of FIG. 42A, 42b, AND 42C, in 2010, the patient was hospitalized for pneumonia. As depicted in row 4215 of the Whole Life tool 4200 of FIG. 42A, 42b, AND 42C, the patient had a surgery, transurethral resection of the prostate, in 2001. In addition, row 4216 of the Whole Life tool 4200 of FIG. 42A, 42b, AND 42C depicts that the patient has had laboratories, illustratively, blood sugars labs were performed, like hemoglobin A1C and update start/stop dates or activate/inactive a diagnosis. It should be noted that in the embodiment of the Whole Life tool 4200 of FIG. 42A, 42b, AND 42C, a valued displayed in some rows and/or columns can be an average value of a measured parameter for the time increment depicted by the column. That is, in some embodiments each row and/or column can be a smart row or column and if a laboratory was taken four times in a year, the Whole Life tool 4200 can be configured to display an average of all values measured during the time increment. In some embodiments, by selecting a value in a row, patient data/information can be displayed in a window or other display means depicting all of the values measured and/or laboratories for the time increment. Even further, by selecting a particular measured value or laboratory, further detailed information for that particular value or laboratory can be displayed to a user/medical provider. Although the embodiment of FIG. 42A, 42b, AND 42C is described as displaying an average value, in some embodiments a high, low or other particular value can be selected by a user to be displayed 4222 represents an alert for an abnormal result.

In row 4217 of the Whole Life tool 4200 of FIG. 42A, 42b, AND 42C, radiological procedures performed on the patient are displayed. For example, in FIG. 42A, 42b, AND 42C, a CT scan was performed on the patient in 2015. In accordance with the present principles, by selectin the indicator in row 4217 of the year 2105, the image of the CT scan can be displayed to the user/medical care provider. In row 4217 of the Whole Life tool 4200 of FIG. 42A, 42b, AND 42C, clinical measurement taken on the patient can be displayed. Such clinical measurement can include blood pressures taken at each doctor's visit. In some embodiments, the results can be displayed as a number. Alternative or in addition, in some embodiments, by selecting an icon associated with the clinical measurements, a graph representing the clinical measurements over time can be displayed. 4219 and 4220 represent radiological procedures and show how they may be toggled between one, many, or all. Images may be directly accessed and viewed within context by selecting them 4284.

In accordance with the present principles, in the Whole Life tool 4200 of FIG. 42A, 42b, AND 42C substantially any portion of a time increment or presentation of patient-related data/information can be selected to cause a display of a more detailed view of the selected time period/value.

In the Medications section (4255) of the Whole Life tool 4200 of FIG. 42A, 42b, AND 42C, start dates and stop dates for each of the medications are displayed and may be interacted with in accordance with Medication Management protocols described herein.

The Whole Life tool 4200 of FIG. 42A, 42b, AND 42C illustratively comprises three optional columns; an alert column 4260, an info column 4265 and a cost column 4270. The alert column 4260 can be used to alert a user/medical care provider of an issue that requires further attention. In some embodiment alerts are automatically created by, for example a Rules module (described in greater detail below), and alternatively or in addition, alerts can be input by the user/medical providers with access to the Whole Life tool 4200.

Whole Life view may be interacted with whereby a doctor may choose to update an event, such as a life event (4290) by selecting said event and the event will auto-populate on the whole life view (4295).

The info column 4265 of the Whole Life tool 4200 of FIG. 42A, 42b, AND 42C can be used to provide information for a user/medical care provider. For example, in some embodiments, links can be provided to direct a user/medical care provider to sources of additional information, such as PUBMED, if the user/medical care provider is interested in learning about medications. Alternatively or in addition, the info column 4265 can be used by users/medical care providers to provide information to other users/medical care providers.

The cost column 4270 of the Whole Life tool 4200 of FIG. 42A, 42b, AND 42C can be used to display to a user/medical care provider information associated with cost in providing medical care a patient. For example, in some embodiments, the cost column 4270 can be used to provide to a user/medical care provider information regarding what a patient's insurance company will authorize. Alternatively or in addition, in some embodiments that cost column 4270 of the Whole Life tool 4200 can display to a user/medical care provider information regarding bills, paid or unpaid, associated with a patient.

In some embodiments, a user/medical care provider can input patient-related data/information into a Whole Life tool of the present principles. Alternatively or in addition, a Rules module can auto-populate patient-related data/information into a Whole Life tool of the present principles. For example, in some embodiments, an integration module of the present principles, such as the integration module 002 of the Data Command Center 001 of FIG. 1, can collect patient data/information from outside sources (e.g., an EMR system). The patient data/information is made accessible, for example via a storage means, to a Rules module of the present principles, such as the Rules module 004 of the Data Command Center of FIG. 1. In addition to having access to the data/information collected by the Integration module 002, the Rules module 004 can have access to all information input by a user/medical care provider via, for example, a medical records dashboard or any other user interface. Alternatively or in addition, in some embodiments, the Rules module 004 is configured to further have access to patient related information and general medical knowledge including but not limited to medical information regarding health conditions and treatments, symptoms and side effects, procedures, images and diagnosis, and other related medical information. As such, in some embodiments, the Rules module 004 can auto-populate at least portions of a Whole Life tool of the present principles. The Rules module 004 can then, via for example a Display module, such as the Display module 006 of the Data Command center 001 of FIG. 1, can cause the display of any portion or zoomed-in portion of a Whole Life tool of the present principles.

In some embodiments, the Data Command Center of the present principles, such as the Data Command center 001 of FIG. 1, can provide, either via a medical records dashboard of the present principles or individually, a Medical Guidance tool to assist users/medical care providers to plan and schedule health services for patients. In some embodiments, the medical guidance tool of the present principles enables a scheduling of patients with automated methodology by, for example, prioritizing the risks of symptoms and diseases, and associating these with past procedures, diagnostic tests and other critical items that need to be evaluated. With such methodology, a medical guidance tool of the present principles guides users/medical care providers in determining, which patients needs the timeliest interventions, appointments and follow up. In some embodiment, the medical guidance tool is configured to examine patient records and information to determine if medications ordered, procedures ordered, follow up visits ordered and if a plan of treatment determined for the patient by a user/medical care provider are accurate or contain any errors.

Alternatively or in addition, in some embodiments a medical guidance tool of the present principles can determine if a patient has missed an appointment and, in response, can alert a user/medical care provider to the fact that a patient has missed an appointment and/or can schedule a task for a user to at least contact the patient to schedule another appointment. In some embodiments, in addition to determining that the patient has missed an appointment, a medical guidance tool of the present principles can determine a level of risk presented to the patient's health by that patient missing the appointment. As such, patient's whose health is at a high risk by missing the appointment can be identified and contacted in an urgent manner to reschedule the missed appointment. In addition, the number of missed appointments can be tracked, whether the patient cancels or the practice cancels, and a pattern identified for the user/medical care provider.

In some embodiments of a medical guidance tool of the present principles, tasks can be generated for different users (e.g., doctors, staff, schedulers, etc) and such tasks can be presented to different users depending on a determined level of risk or urgency to a patient. For example, doctors typically do no schedule follow up appointments for patients. Such task is usually performed by a scheduler. As such, typically scheduling tasks generated by a medical guidance tool of the present principles are generally directed to an identified scheduler. In some embodiments however, if a patient misses an appointment and the a medical guidance tool of the present principles determines that missing the appointment presents an elevated risk to a patient's health, the medical guidance tool of the present principles can generate a rescheduling task that is now directed to the doctor. Alternatively or in addition, the medical guidance tool of the present principles can generate an alert to be present to a user/medical care provider that the missed appointment presents an elevated risk to the health of the patient.

For example, in a scheduling embodiment, an integration module of the present principles, such as the integration module 002 of the Data Command Center 001 of FIG. 1, can collect patient data/information from outside sources (e.g., an EMR system). The patient data/information is made accessible, for example via a storage means, to a Rules module of the present principles, such as the Rules module 004 of the Data Command Center of FIG. 1. In addition to having access to the data/information collected by the Integration module 002, the Rules module 004 has access to all information input by a user/medical care provider via, for example, a medical records dashboard, such as the medical records dashboard 400. In some embodiments, the Rules module 004 is configured to further have access to patient related information and general medical knowledge including but not limited to medical information regarding health conditions and treatments, symptoms and side effects, procedures, images and diagnosis, and other related medical information. As such, in some embodiments, the Rules module 004 can monitor patient data/information and can be configured to monitor patient scheduling. As such, when a Rules module 004 determines that a patient has missed a scheduled appointment, by for example determining if a user/medical care provider has interacted with the patient that day or not by determining if any information has been entered into a medical records dashboard or other user system for that patient that day, a Rules module 004 can determine if a patient has missed a scheduled appointment. If the Rules module 004 determines that a patient has missed a scheduled appointment, the Rules module 004, via for example a Display module of the present principles, such as the Display module 006 of the Data Command center 001 of FIG. 1, can cause a display of an alert, to call to the attention of a user/medical care provider that the patient has missed a scheduled appointment. Alternatively or in addition, the Rules module 004 can cause the scheduling of a task to be presented to a user/medical care provider such that a new appointment can be scheduled for the patient.

In some embodiments, having information regarding at least patient medical conditions, general and specific treatments and procedures, patient scheduling and other patient-related data/information, the Rules module 004 is able to determine if missing the scheduled appointment place the patient's health at an elevated risk. If so, the Rules module 004 can cause, for example via the Display module 006, a display of an alert, to call to the attention of a user/medical care provider that the patient's missed appointment results in an elevated risk to the patient's health. As described above, the determination of the elevated risk can cause the alert to be directed to a higher-level user such as a doctor instead of an administrator. In some embodiments of the present principles, the display of the alert itself can change and can be caused to be presented in a different color than usual or with other visual attributes, such as blinking or appear large on a display.

In some embodiments, a Medical Guidance tool of the present principles can assist in the scheduling of an appointment for a patient. For example, in an embodiment in which a scheduler is inputting patient data/information into an electronic system/spreadsheet/form, the Rules module 004 of the present principles can be configured to monitor such input patient data/information. Using the monitored input data/information and medical information known to the Rules module 004, the Rules module 004 can cause a display of a suggested appointment date to a user. For example, if a patient is known to have had a procedure performed and such procedure has a post-operative appointment typically scheduled for 30 days, the Rules module 004 can cause a display of a suggestion to a user that an appointment be scheduled for 30 days after the procedure was performed. In some embodiments, for suggesting an appointment, the Rules module 004 can further consider parameters such as time since a last procedure, symptoms since the last procedure, the doctor that performed the last procedure, medical history of the patient, the patient's disease state, and the like. In some embodiments, for new patients, the Rules module 004 can even take into account, who is referring the patient. If a patient referral comes from a doctor in a subspecialty that clearly would know what is an emergency, like another eye doctor, the Rules module 004 might suggest that an early appointment date must be made. In some embodiments, the Rules module 004 can create tasks for a user to make appointments on a suggested date or alternatively or in addition can schedule appointments without the need for a user intervention.

In some embodiments of the present principles, a Medical Guidance tool can assist in scheduling a patient to see a different doctor than the patient came to see. By way of example, in ophthalmology there may be in one office a general ophthalmologist, an optometrist, a retina surgeon and a glaucoma surgeon. A patient with diabetes and glaucoma may need to see the retina surgeon four times a year and the glaucoma surgeon three times a year. A scheduler for the practice or even the patient themselves can get confused as to which doctor to see. For instance, if the glaucoma doctor sees the patient and does not schedule the patient to return to the retina doctor, who handles another type of disease, not infrequently, patients can be totally lost and the wrong provider is assigned to give care. While one provider may be taking care of one disease state, (i.e. glaucoma), the other states (i.e., diabetic eye disease or macular degeneration), can be inadvertently neglected. The same can be true in a multispecialty practice of internists, cardiologists and pulmonologists. For example, an internist can have a good working relationship with the patient and sees the patient on a regular basis, however, the internist may not realize that the patient did not keep or ever get scheduled for an appointment with a cardiologist.

In some embodiments in accordance with the present principles, the Rules module 004, having knowledge of a patient's entire medical history, conditions, current procedures and treatments and having general knowledge of medicine and specifically the relationship between treatments and procedures of internists and cardiologists, can cause a display, for example via the display module 006, of at least one of an alert, suggestion and/or a task that causes the patient to be scheduled for an appointment with a cardiologist. A Medical Guidance tool of the present principles is able to determine when a patient is supposed to return for an appointment, what the high-risk scenarios exist, whether there was a procedure performed on a patient that requires a follow up with a particular doctor, whether a follow up appointment is kept by the patient, and is able to suggest or remind a user/medical care provider that they should consider sending a patient to another doctor. In some embodiments, not only are there indicators and alerts sent to the doctor who sees the patient, but indicator and alerts can be sent to an original doctor whose appointment has been missed, to a practice manager, or to anyone else in the practice to be able to determine whether a particular patient should be seeing a particular doctor or if the patient has been lost in the shuffle of so many visits. Such mistakes can happen in health systems and hospitals in which a patient who is not knowledgeable about medicine makes the assumption that each doctor talks to one another and shares records and therefore the patient assumes that if one doctor does not suggest that the patient sees another doctor, that the original doctor will be taking care of everything and the patient does not need follow-up care from a different doctor.

In some embodiments, a Medical Guidance tool of the present principles can monitor patient-related information intended to be reviewed by at least one user/medical care provider to determine if that information has been reviewed by the at least one user. For example, in some embodiments, the Medical Guidance tool, for example via the Rules module 004 can identify if results from tests ordered or notes from other medical care providers or any other “attachments” sent to for example a medical records dashboard, have been reviewed by all intended users/medical care providers for which they were intended. If the patient-related data/information intended for review by users/medical care providers has not been reviewed, the Medical Guidance tool can cause a display of an alert to the users/medical care providers that have not reviewed the patient-related data/information and for which the patient-related data/information was intended. Alternatively or in addition, the Medical Guidance tool can create a task for at least one of users/medical care providers for which the patient-related data/information was intended and whom have not reviewed the patient-related data.

For example, in a multispecialty practice, if the pathology results of a biopsy of a skin lesion was received and a family doctor sees the results, but the dermatologist who ordered the biopsy does not see the results, an alert can be sent out to either one or both of the doctors or alternatively or in addition, a task can be created for one or both of the doctors to view the results of the biopsy.

In some embodiments, a Medical Guidance tool of the present principles enables the pre-analysis of current and future patent visits. Such functionality enables users/medical are providers to prepare for patient visits and review scheduling of patients and test/procedures to determine if any errors exist. A user/medical care provider can review tests/procedures scheduled for a patient, when the patient last had similar tests, what patient's disease states are, what the likelihood is that the patient might need additional tests or another type of procedure, and even whether or not something might have been scheduled in error because information from the previous visit doesn't match up. For example, if a patient treatment plan indicates that an injection is to be done in the left eye but the schedule says injection in the right eye, the Medical Guidance tool via, for example, the Rules module 004, can discover the discrepancy and cause an alert to be displayed to a user/medical care provider to warn of the discrepancy.

In some embodiments, a Medical Guidance tool of the present principles enables the post-analysis of patent visits. Such functionality enables users/medical care providers to pull up patient data/information related to visits of any past patients seen in any office or a particular patient seen with certain disease states or procedures or diagnostic tests and see what was done on any given time period or visit. Such functionality can be especially useful if a user/medical care provider references, for example, a medical records dashboard on the same day of a visit or shortly thereafter when the memory of patients are fresh in their memory. During such review, a user/medical care provider can review to determine if their examinations were filled out correctly and that any diagnostic and/or procedural matters were performed and performed correctly and determine if any tests or orders were missed.

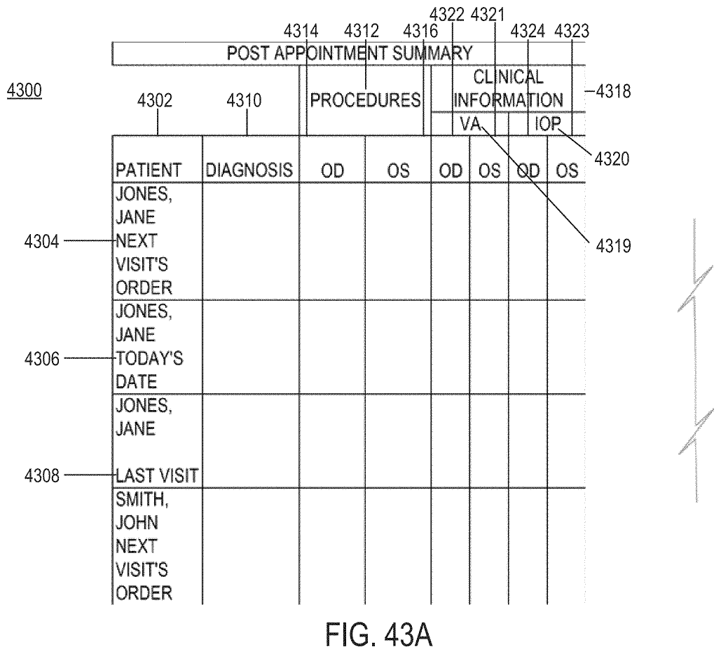

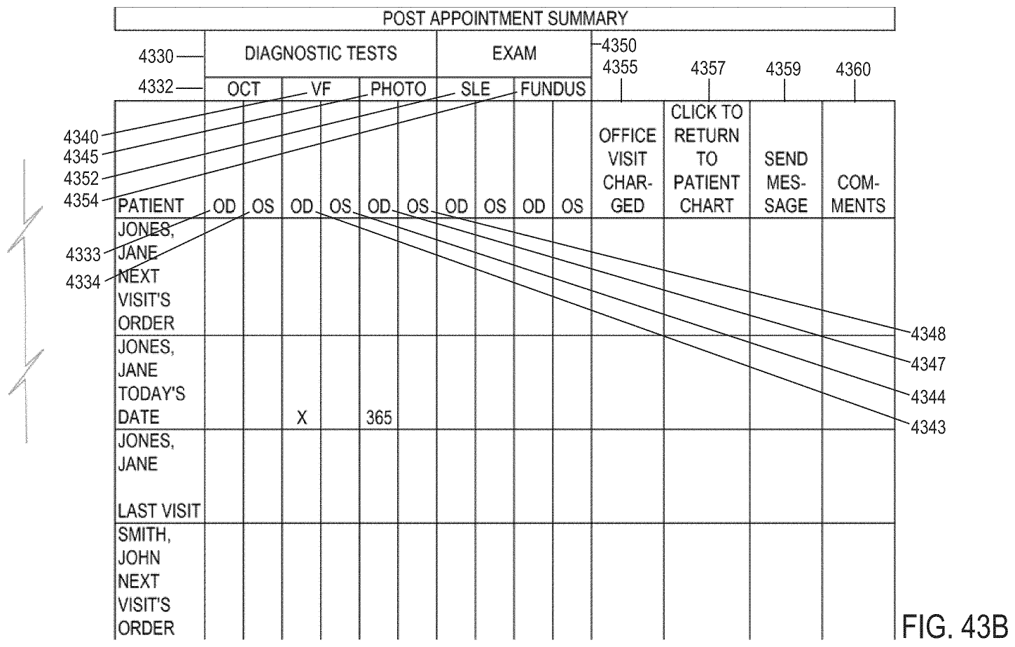

FIGS. 43A and 43B depict a post appointment summary chart 4300 of a Medical Guidance tool in accordance with an embodiment of the present principles. The post appointment summary chart 4300 illustratively includes a first column 4302 listing a next visit's order row 4304, a today's date visit row 4306, and a last visit's row 4308. The post appointment summary chart 4300 has a plurality of other columns including a diagnosis column 4310, a Procedures column 4312 including a right eye column 4314 and a left eye column 4316, a Clinical information column 4318, including a VA column 4319 and an IOP column 4320, each having respective right eye columns 4322, 4324, and respective left eye columns 4321, 4323, and a Diagnosis Tests column 4330, including an OCT column 4332, a VF column 4340 and a Photo column 4345, each including respective right eye columns 4333, 4343, 4347, and respective left eye columns 4334, 4344, 4348.

The appointment summary chart 4300 of FIGS. 43A and 43B further illustratively includes an Exam column 4350 including an SLE column 4352 and a Fundus column 4354, an Office Visit charged column 4355, a Click to return to PT chart column 4357, a Send message column 4359, and a Comments column 4360. Although in the embodiment of FIGS. 43A and 43B the appointment summary chart 4300 illustratively includes specific columns and rows for providing the illustrated patient related data/information, in some other embodiments different patent related data/information can comprise the appointment summary chart 4300. In addition, although in the embodiment of FIGS. 43A and 43B the appointment summary chart 4300 illustratively comprises a post appointment summary chart of the present principles, the appointment summary chart 4300 can comprise a pre appointment summary chart in accordance with the present principles. In some embodiments, at least some of the rows and columns of the appointment summary chart 4300 can be auto-populated.

In some embodiments, a Medical Guidance tool of the present principles enables users/medical care providers to create a preferred practice method. For example, in some embodiments, a Rules module 004 is programmed with a preferred practice method of a user/medical care provider. The Rules module 004 can then provide services, such as assisting in the creation of appointments and determining if patients kept their scheduled appointments in accordance with the preferred practice method of the user/medical care provider.

In some embodiments, a Medical Guidance tool of the present principles enables a tracking of payments in accordance with the present principles. Current EMR systems require user driven reports to be run manually to identify items that have not been paid or are rejected by insurance carriers. Most insurance companies send payment for hundreds of separate patient claims on the same electronic check that is then posted automatically to many different patient accounts without inspection or review by a billing or staff member. This electronic process was developed to reduce workloads on staff who before were required to read the explanation of benefits and apply the payment manually to each individual claim item in the billing system, which allowed for greater oversight of incorrect payments and rejections.

In some embodiments of a Medical Guidance tool of the present principles, a Rules module, such as the Rules module 004 of the Data Command center 001 of FIG. 1, can be configured to monitor individual CPT codes in, for example in some embodiments, a medical records dashboard of the present principles, to identify when bills are not completely paid or are rejected. In some embodiments, if it is determined that a bill is not completely paid or rejected, the Rules module 004 can cause, for example via the Display module 006, a display of an alert to alert a user/medical care provider that a bill was not completely paid. Alternatively or in addition, a task can be created for a user/medical care provider to correct the unpaid bill. In such embodiments, the Rules module 004 can have access to such information as specific insurance payors information, patients with high deductible plans, amount of billing, and the like. All pertinent data can be analyzed by, for example, the Rules module 004 and an indicator or a task can be created to alert the appropriate staff members and physicians enabling the users to make corrections rapidly. In some embodiments, based on user preferences, fully automated queries can generate indicators that can be viewed live while a patient is being treated.

In some embodiments of the present principles, a Medical Guidance tool of the present principles provides an electronic patient interface. For example, when a patient calls for an appointment or to ask questions or emails to schedule an appointment or ask questions, a user interface enables a patient to ask and answer questions, enter information, refill medications and the like. The reality is doctors often do not have the time to communicate with each and every single patient. In some embodiments, a Rules module of the present principles, such as the Rules module 004 of the Data Command center 001 of FIG. 1, having knowledge of all patient related data/information is also provided access to all information provided by a patient via the caller or email user interface. The Rules module 004 can evaluate every patient query in light of the information available to the Rules module 004. In some embodiments, the Rules module 004 determines if the patient is a current patient and if so, if the patient h had procedures or a risky diagnosis so that the Rules module 004 can present to a user/medical care provider a most complete picture of a patient as possible including which patients might be more problematic and urgent based on patient's symptoms, diagnosis, past procedures and other patient history In some embodiments the Rules module 004 can generate an alert directed to a user/medical care provider, the alert including the details of the patient developed by the Rules module 004. Alternatively or in addition, the Rules module 004 can create a task for a user/medical care provider, the task including the details of the patient developed by the Rules module 004. If the alert/task is not responded to within a certain amount of time by the user/medical care provider, the Rules module 004 can generate another alert and/or task directed to another user/medical care provider to attempt to elicit a response for the patient.

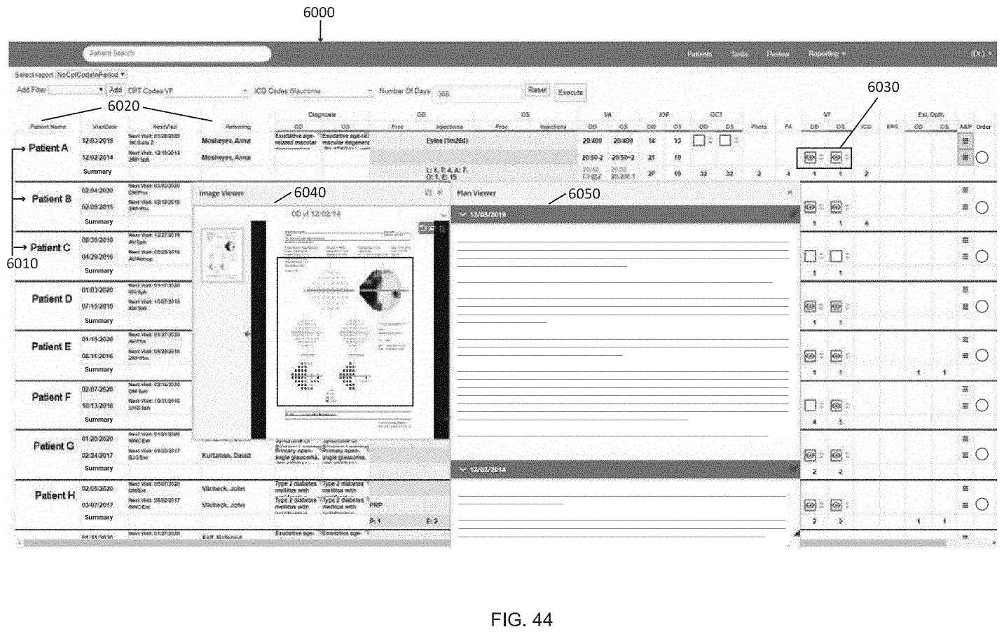

In some embodiments of the present principles, a Data Command Center of the present principles enables an organized view derived from disparate data sources in accordance with user-defined parameters, of a specific report type, executable on a single or group of patients. For example, FIG. 44 depicts an Evaluative Clinical Reporting (ECR) interface 6000 that provides an organized view derived from disparate data sources in accordance with user-defined parameters, of a specific report type, executable on a single or group of patients. The ECR interface 6000 displays only necessary and relevant data based on individual patient information in rows 6010 and columns 6020 that allows Clinical Professionals the ability to evaluate many data points and make informed clinical decisions, which would not otherwise be possible in a single display. While reviewing the data, the Clinical Professional can execute multiple actions, including but not limited to the following:

First, the Clinical Professional can, with a single click, view additional relevant data by clicking on icons 6030. An example of this would be viewing diagnostic imaging in a separate, overlaid panel 6040, from the relevant visit date. It is important to note that while viewing the diagnostic imaging, the Clinical Professional can manipulate the underlying flowsheet while manipulating the diagnostic image.

An additional example of this functionality would be viewing the plan 6050 that was used for that specified visit. As with the previous example, the underlying flowsheet can still be manipulated when viewing the plan 6050. It is important to note that N number of additional data panels can be simultaneously opened and overlaid on the reporting results.

A clinical professional can also use a single click on an icon 6030 to load a specified letter for viewing in a separate, overlaid panel, while still able to view the flowsheet in context. As with the previous examples, the Clinical Professional is able to manipulate the flowsheet and also view additional relevant data panels by clicking an icon on the flowsheet while viewing the letter.

After the Clinical Professional has reviewed the patient, there may be some action to take in order to rectify whatever is causing the patient to be included in the report. The Clinical Professional may choose to create a task for a support staff member to take some action. The Clinical Professional may also choose to create an order for a patient while viewing the report results. An ordering panel may be launched within context by clicking an icon. The panel is overlaid with the reporting results flowsheet. As described, the panel provides a mechanism by which the Clinical Professional can select the type of order, input the relevant parameters and then create the order. A Clinical Professional may also choose to create a letter to a Referring Provider in order to satisfy regulatory requirements or provide guidance to the referring provider in a co-management situation. The Clinical Professional would launch the create letters interface over the top of reporting results flowsheet allowing them to write a letter in context of the flowsheet data on screen. However, it is possible that the Clinical Professional does not need to take any action and simply chooses to evaluate the many data points in a concise view of rows and columns of relevant data.

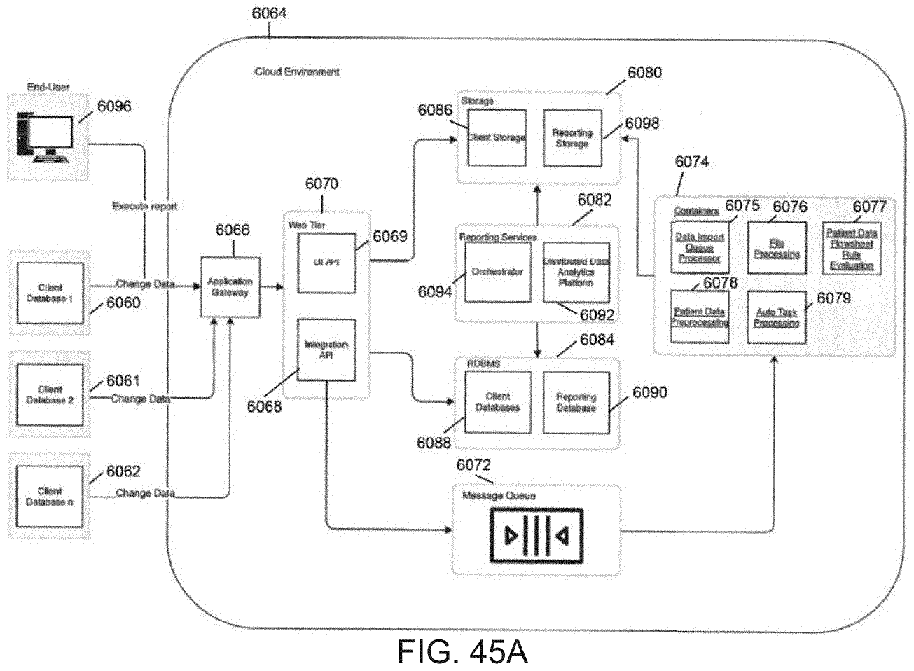

FIG. 45 depicts a reporting architecture of Data Command Center in accordance with an embodiment of the present principles. As data changes on a remote client's EHR 6060, Practice Management 6061, Diagnostic Equipment 6062, or other system, it is pushed to the cloud environment 6064 via an application gateway 6066 to an Integration API server 6068 in web tier 6070. Once the Integration API server 6068 receives the HTTP request with the updated data, it will then insert a message into the appropriate message queue 6072. Five separate application containers 6074 are connected to the message queue 6072 waiting to process information as it is received. A container is defined as a method to package an application, all its dependencies, and runs in isolation from other processes.

The first application container, Data Import Queue Processor 6075, processes changed data and loads it into the appropriate client database 6080. During the processing, the data is mapped to the correct database entities by reporting services 6082 and inserted into the Client Relational Database Management System (RDBMS) 6084. Once the entities are inserted into the client database 6086, a message is added to a Patient Data Preprocessing queue. Placing this message into the queue will cause the Patient Data Preprocessing Queue to begin processing for the patients whose records were updated.

The second application container, File Processing 6076, loads all new diagnostic imaging into Client Storage 6086. Additionally, a record will be added to the Client Database 6088 which contains a pointer that allows for the UI API 6069 to retrieve the correct image for the patient.

The third application container, Patient Data Flowsheet Rule Evaluation 6077, queries the client database 6086 for the latest snapshot information, including, but not limited to, Demographic Information, Billing History, Diagnostic Imaging, and Medical Data. Once the relevant information has been loaded into memory, a series of rules are executed in order to transform those data into a JSON object optimized for displaying Medical Information, Diagnostic Testing results, etc. in rows and columns of the flowsheet. Once all rules have been run, the Flowsheet Encounter Data is then written to the Client Storage File System 6086.

The fourth application container, Patient Data Preprocessing 6078, will generate an object that contains a historical snapshot of everything that has happened to the patient including, but not limited to, Demographic Information, Billing History, Diagnostic Imaging, and Medical Data. This snapshot, Patient Data Transfer Object (Patient DTO), is stored in a JSON object and written to the Reporting File System 6090 which will be used during the Extract, Transform, and Load (ETL) process to insert rows into the Reporting Database. The ETL process consists of multiple steps to Extract the data from the client RDBMS 6084, Transform it with logical commands executed by the Data-Analytics platform 6092 which is distributed to multiple worker nodes by Orchestrator 6094, and Load the data into the Reporting Database 6090.

The Reporting Database 6090 includes a series of Facts and Dimensions tables which can be extended to include new tables as new use-cases are encountered. Those skilled in the art will appreciate that the data may be architected to use a Star Schema in order to ensure that data is able to be returned in an acceptable time. All the data is stored in a single database. In order to ensure that data is kept appropriately segregated per client, all tables contain a Customer Id Unique Identifier column. The defined process to generate new reports mandates that Report Developers write all queries to include a Customer ID parameter in the WHERE clause, ensuring that data will only be returned for the customer that is specified. In order to be able to view the report execution interface, the user must complete the login, or authentication, process. The authentication process requires a practice identifier, a user, and a password be passed as parameters to the authentication controller. Once authentication has completed, a Secure, HTTP Only, Same Site cookie is generated by the Integration API server 6068 which contains a claim that ties the current authenticated user, to a customer ID. When the report execution HTTP request is made, the cookie is passed in the request, the customer ID is extracted from the cookie and passed automatically as a parameter to the stored procedure execution.

Finally, the Auto Task processing container 6079 runs a set of defined rules to evaluate for certain conditions and to generate a task for a patient once certain conditions are met. If it is found that a certain condition has exceeded a threshold, a task will automatically be sent to the appropriate user to handle the task. For example, if a patient has a diagnosis that requires a certain diagnostic test every 365 days and has exceeded that threshold of 365 days, a task will be sent to the computer 6096 of the Front Desk Staff to schedule that diagnostic test. Once the patient has the required diagnostic test, change data will trigger the reprocessing of the auto task rules, and will automatically resolve the task for the relevant patient.

In order to move the newly generated files from Client Storage 6086 to Reporting Storage 6098, a time-based job executes on a scheduled interval (cron job) on the order of minutes, to find all new and/or modified Patient DTOs in the Client File System since the last time the job was run, and then places a replica of the newly modified file in the Reporting File System of the Reporting Storage 6098. These jobs will continually copy new or modified files to the Reporting File System as the Patient DTOs are generated.

During the day, Patient DTOs will be moved from Client Storage 6086 to Reporting Storage 6098, waiting for its data to be loaded into the reporting database 6090. In order to start the ETL process, another cron job starts the execution of the Orchestrator Master Pipeline which contains all its child pipelines. The interval for execution is generally set to each night. The master pipelines specify the order in which the child pipelines should execute, error handling, and other logical functions. The pipelines will load the base changed data from the clients' database to create a batch as depicted at 6100 of FIG. 45B.

Base changed data consists of, but is not limited to, patient demographics, patient insurances, customers, providers, practice locations, etc. The orchestrator pipeline allows the addition, or removal, of pipelines at 6102, 6104 to extend the ability to transform and load new facts and dimensions. While the base data is loaded, an additional pipeline 6106 executes concurrently which will process the Patient DTO data. Each child pipeline 6108 is processed by calling separate transformation jobs which contain the necessary logic to transform the Patient DTO data for insertion to the relevant Facts and Dimensions tables at 6110. The transformation logic is applied using the distributed data-analytics platform 6092. After the Orchestrator Pipeline 6094 has finished processing, the data is logged at 6112 and is available to be reported on.

Once the ETL process begins, the Patient DTO data is staged to be transformed and loaded to the reporting database. To begin the Transformation process, the most recent JSON schema for the Patient DTO object is loaded into memory. The schema is then used to read the DTO object from reporting storage into a proprietary table common for the data-analytics platform 6092. This object allows, but is not limited to, filtering out certain values, adding new columns, dropping unnecessary columns, and flattening child arrays. Due to the nature of the data coming from various source systems, with their own constraints for data-integrity, it is expected that some of the data will not be in a state where it can be loaded into the reporting storage. These data, dirty data, must be scrubbed and cleaned, or be discarded for later analysis. If the data has been discarded, an alert is sent, within the reporting system that data has been dropped and inserted into the dropped data table and requires investigation for the reporting developers. If the data can be scrubbed, then it is transformed in order to meet the minimum constraints of the reporting database. Once the data is sufficiently clean, rows in the proprietary table object which contain child data in arrays, must be exploded, or flattened, to create additional rows to be inserted.

Consider the following:

A table is defined to have two columns, X and Y

The table contains one row with values of [{X: 1, Y: [1,2]}]

The table is then flattened, specifying Y as the column to flatten

The output of the table is now two rows [{X:1, Y:1}, {X:1, Y:2}].

If the table contains multiple arrays, it may be necessary to flatten the table multiple times. Once the table has been appropriately flattened, columns may be Renamed, Dropped if they are not needed and/or Added with static values including, but not limited to, batch ID, customer ID, etc. or with dynamic lookup values.

After the data has been transformed, it is then inserted into the reporting database 6090 to be used in the report execution process which queries two sources, the reporting database 6090 and the client storage 6086 which contains the most recently cached flowsheet encounter data in order to display the results in the UI on the end-user computer 6096, for example.

During use, an Interface a user is enabled to select a report from a dropdown list. Once the report has been selected, the UI will make an HTTP request for the available parameters for the report specified from the UI API. Examples of the parameters which can be used include, but are not limited to, procedure codes, diagnosis codes, number of days since a procedure occurred, clinical data elements, and next appointment status. After the UI receives the response to the HTTP request, a list of parameters is then displayed. This list allows the end-user to dynamically add parameters for the specified report. Those skilled in the art will appreciate the ability to select multiple parameters using Boolean logic to specify whether all the parameters must match (AND Logic) or only some of the parameters must match (OR Logic). It is known that not all the parameters are required but there will always be at least one required parameter. Until all the required parameters have been added and set by the end-user, the report cannot be executed.

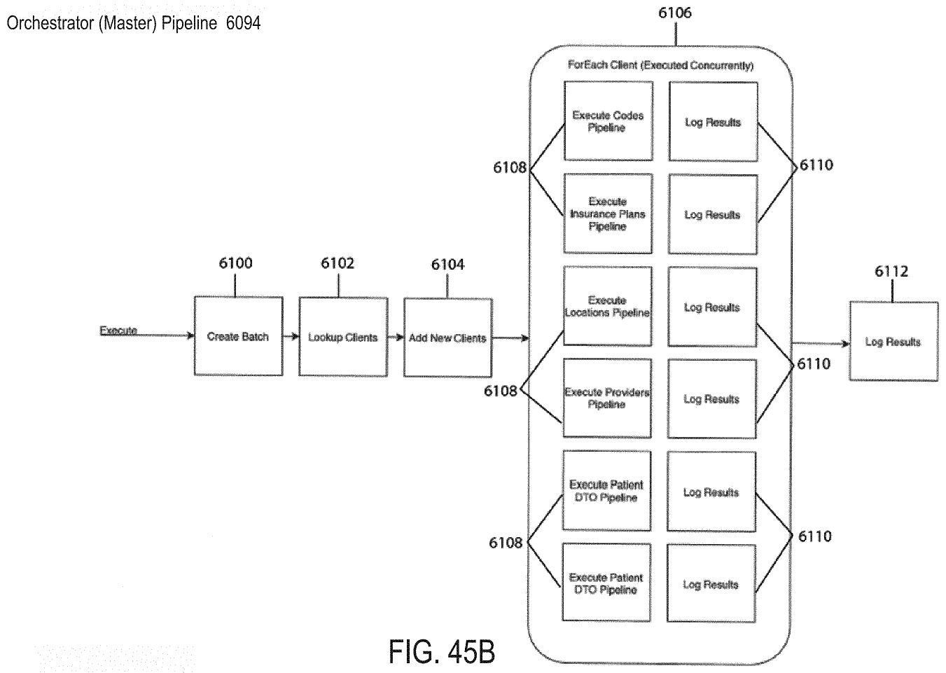

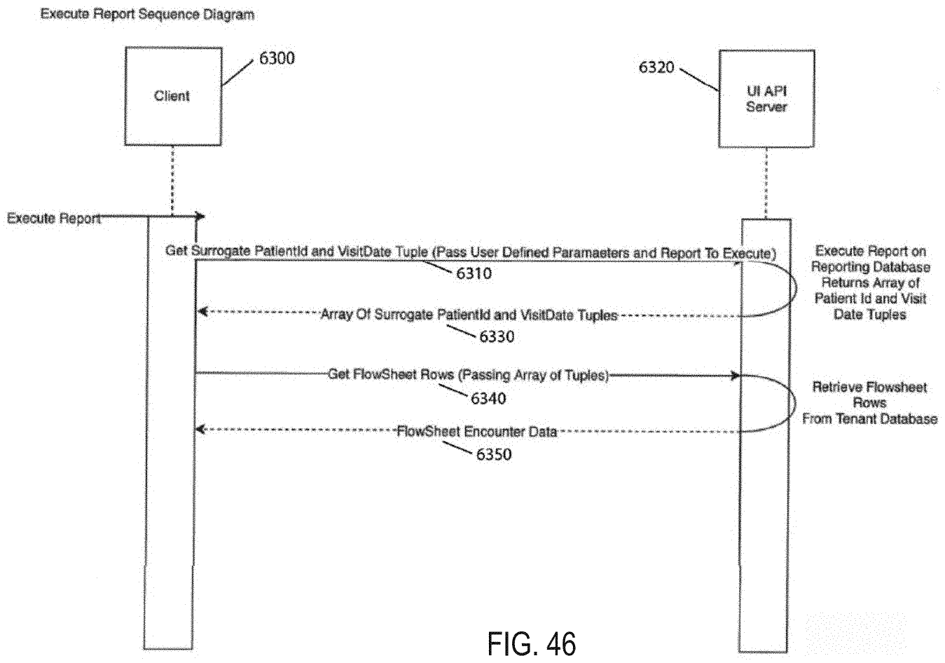

FIG. 46A depicts a sequence diagram for executing a report in accordance with an embodiment of the present principles. Once all the required parameters have been added, the end-user (client 6300) may then click a button to generate, or execute, the report HTTP request with the user-defined parameters. After the button to execute the report has been clicked, the UI app of the client 6300 will make an HTTP request 6310 to a controller in the UI API server 6320. The controller is responsible for setting the correct customer ID context, ensuring that the current user has access to the specified report, and calling the correct Stored Procedure in the Reporting RDBMS 6084 of FIG. 45. The Stored procedure executes a series of queries and returns an array of Visit Date and Patient ID tuples 6330. Once the Stored Procedure has finished execution, the array of Visit Date and Patient ID Tuples is returned to the UI App. The UI App then makes another HTTP request 6340 to another controller in the UI API passing the list of Visit Date and Patient ID Tuples. The controller then executes a stored procedure on the Client Database which returns Flowsheet Encounter Data filenames 6350 for the specified visit dates. Once the list of filenames are returned to the controller, the application will loop through all of the unique patient IDs in the array of tuples that was passed as a parameter, and load the encounter data using the specified filenames retrieved from the stored procedure. Once the encounter data has been loaded from the filesystem, a summary row is generated which contains summary information for all columns specified in the flowsheet. An example of summary information could be the number of injections, by type, listed in the procedure column. Another example of summary row information could be for a patient's Visual Acuity (VA).

It is often helpful to know what a patient's best VA or worst VA over the course of care. The summary row would analyze all the historical data for a patient and insert the patient's worst VA and best VA into the summary row allowing the provider to view and evaluate years' worth of data in seconds. In order to evaluate years' worth of data without a summary row, a Clinical Professional would need to undertake the time consuming task of reviewing all Vision Tests, or Refractions, a patient has had, often having multiple refractions per visit, sometimes over the course of 10+ years of treatment. Finally, the API will then return an array of flowsheet data to the UI app which represents the group of patients which match the report type and user-defined parameters.

The UI app will then process the flowsheet data and render the relevant data in the appropriate rows and columns as shown, for example, in FIG. 44. The columns that are displayed are configurable based on the type of procedures, diagnoses, next appointment status or date, etc., selected as parameters for a report. Based on the parameters specified, an HTTP request will be made to the Flowsheet configuration controller, which will return the flowsheet configuration which contains the columns to display. This allows the Clinical Professional the ability to only see the most relevant data for the group of patients that they may be reviewing.

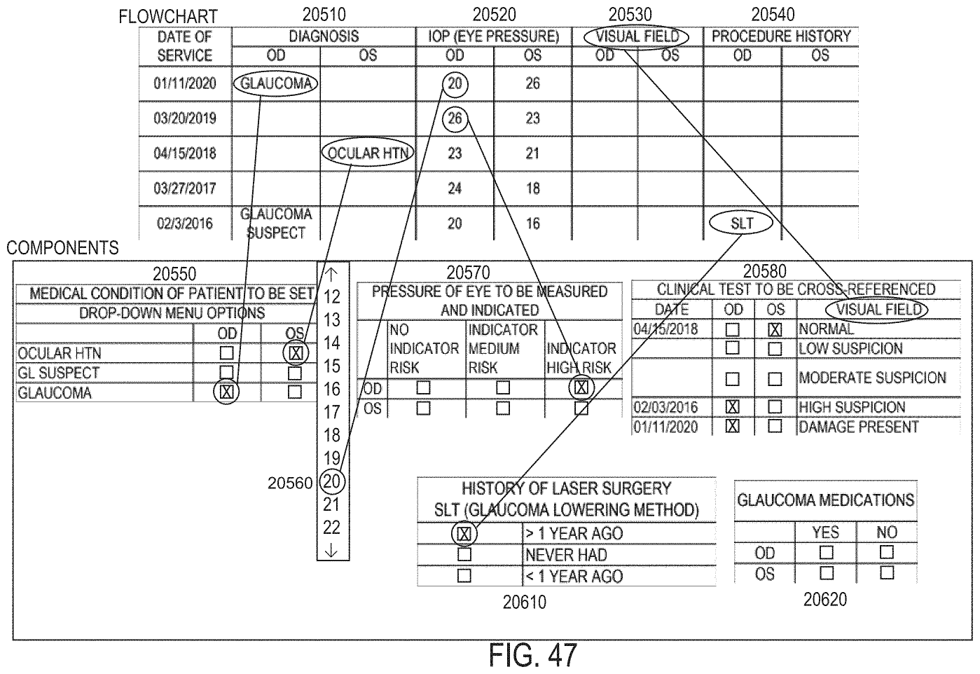

In some embodiments, a Data Command Center of the present principles enables the configuring of alerts in, for example, a medical records dashboard of the present principles. FIG. 47 depicts an embodiment of a medical records dashboard of a Data Command Center in which alerts and tasks can be performed in accordance with an embodiment of the present principles. Indicators such as diagnosis 20510, key results 20520, diagnostic tests 20530, and procedures 20540 are all key factors which may be used to trigger an alert or to send an automated task. In the diagnosis column 20510, glaucoma is indicated for the right eye (OD) and Ocular HTN is indicated for the left eye (OS) as required within the medical conditions dialog box 20550 as triggers. Thresholds are selected from a dropdown menu for key results 20560, for the results column 20520. Visual indicators may be selected 20570 to alert users of a threshold being met, exceeded, or falling below the specified level. A task panel 20590 may be utilized to set parameters at which an automated task will be sent and may involve triggers and time delays as to when the task will be sent 20600. Clinical tests may be validated and cross-referenced 20580 against a variety of conditions 20550, thresholds 20560, events 20610, and relevant factors 20620 to intelligently determine if an alert or task would be created.

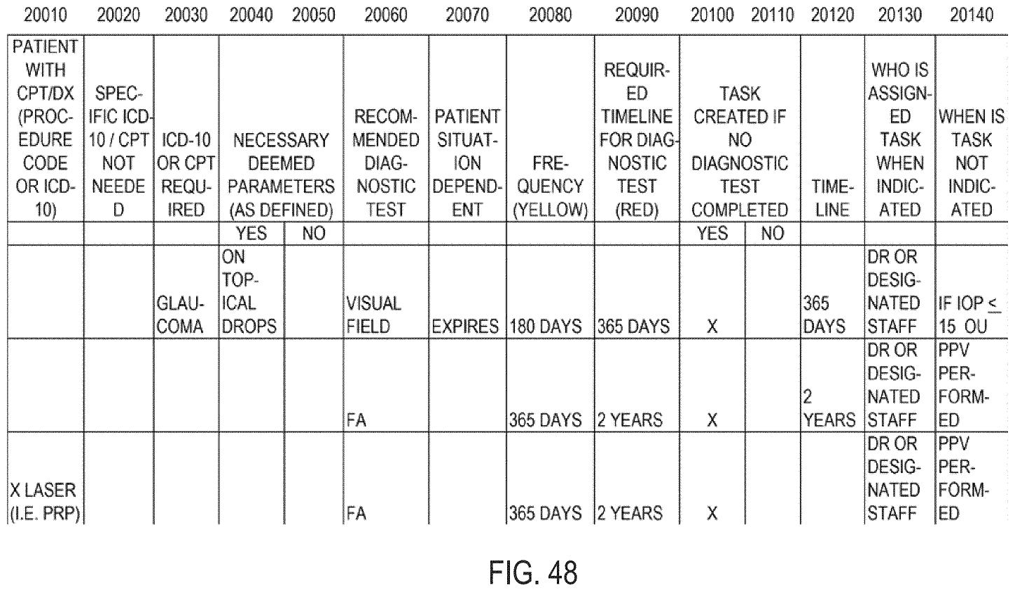

FIG. 48 depicts a menu for pre-configuring alerts in accordance with the present principles. That is, in some embodiments, alerts can be configured ahead of time or at point of care for individual or groups of patients. An example of configuring an alert is shown in FIG. 48. Parameters are defined such as including specific diagnostic or CPT codes 20010, ignoring specific diagnostic or CPT codes 20020, required specific diagnosis or CPT codes 20030, parameters deemed necessary which may be necessarily true 20040 or false 20050, recommended diagnostic tests 20060, patient dependent situations 20070, defined by frequency in yellow 20080, have a time interval within which to be performed in red 20090, tasks may trigger if required diagnostic test is not completed 20100/20110, be aligned to a timeline 20120, have tasks auto-generated to a specific person or group 20130, and exceptions which prevent tasks from sending 20140.

In this example, a Laser or Injection can be a trigger in the first column 20010. Glaucoma and Glaucoma Suspect would be required parameters in the third column 20030. The patient being on topical drops is a requirement deemed necessary in the first half of the fourth column 20040. Visual field, photo, and FAs are recommended tests in the fifth column 20060. Expiry and expiry based on seeing a retina specialist are examples of patient situation dependencies 20070. Frequency may be defined in yellow for any time increment 20080. Required Timeline may also be specified in red for any time increment (20090). Tasks are specified to be created 20100 or not 20110 based on completion of said diagnostic test. Timeline may be specified in any time increment (20120). An individual or group is chosen as assignee 20130. Finally, the task trigger may be ignored based on specific criteria such as an IOP threshold or action taken 20140.

In accordance with the present principles, substantially any portion of a display, such as a medical records dashboard of the present principles, can be configured for an alert, including, but not limited to column headers and summaries, individual values within rows and/or columns, whole rows and/or columns, module headers, values displayed within modules, whole modules, and general application alerts outside of rows, columns, and/or modules. In some embodiments, alerts can be configured in accordance with Clinical Trials. Clinical trials exist throughout medicine often to determine efficacy of treatment and can include any number of other validations. As defined, alerts and auto-tasks can be configured for individual patients or groups of patients. In the specific case of clinical trials, a group of patients can be defined with specific criteria. As alerts can be configured by condition, procedure, risk factors, demographics, and a variety of other reasons, alerts can be used to isolate a group of patients deemed eligible for clinical trials.

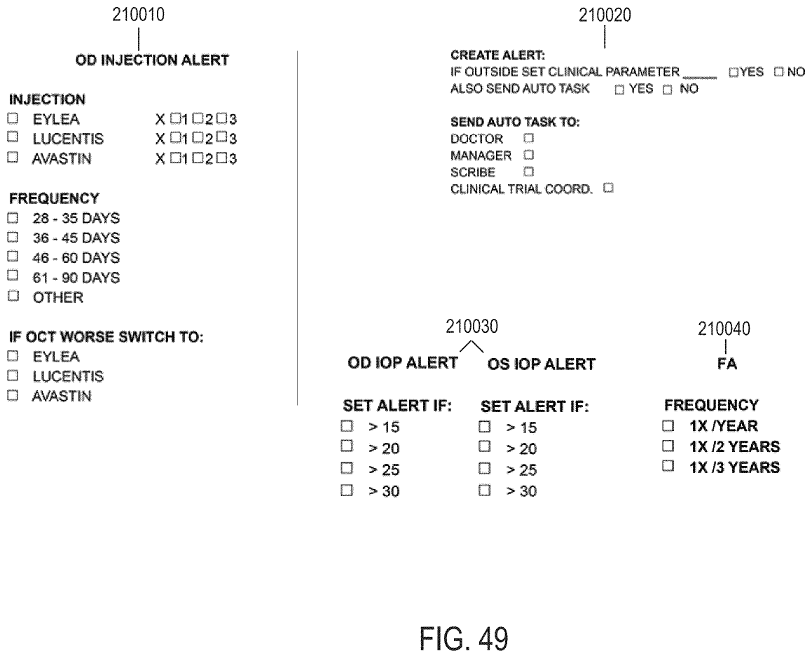

Often, clinical trials are based on medication efficacy. FIG. 49 depicts an embodiment of a medical records dashboard in which alerts are configured based on medication. As seen in FIG. 49, alerts can be configured based on medication, instance count of medication, frequency of medication, and can also have secondary or tertiary responses to the outcome 210010. Alerts can be configured based on defined parameters for clinical trials and may send automated tasks in the case of a patient or patients falling outside defined parameters 210020. Results can have predefined thresholds to trigger alerts and/or automated tasks 210030 and procedures, diagnostics 210040, or lack thereof, can also trigger an alert or automated task. Additionally, required actions such as a diagnostic test 210040 can be required within a specific amount of time. One skilled in the art will appreciate that the ability to preconfigure rules, alerts, notifications, and tasks, in accordance with clinical trials is an example which can be applied across multiple areas of research and healthcare. The inclusion of financial decision support also expands this functionality to be of interest to pharmacological, insurance, and other entities and organization to denote key requirements for said entities, whether for a single patient, group of patients, group or groups of patients within specific parameters.

In some embodiments, if a patient misses an appointment, an auto task can be generated to alert a user/medical care provider schedule that the patient missed the appointment so that another appointment can be scheduled. or to the Clinical trial coordinator if it is part of a research protocol. Even parameters of when to create the task such as two missed appointments in a row can be set. This enables automatic tracking and a user/medical care provider can set it knowing the unique individual issues with a patient and can determine how important a missed appointment might be for a particular patient at a glance by showing previous data projected in the background or through one user interface on another monitor the user/medical care provider can cross check and individualize the alerts and tasks since a single missed appointment may be serious for one patient, but not so serious for another. Even parameters of when to create the task such as two missed appointments in a row can be set.

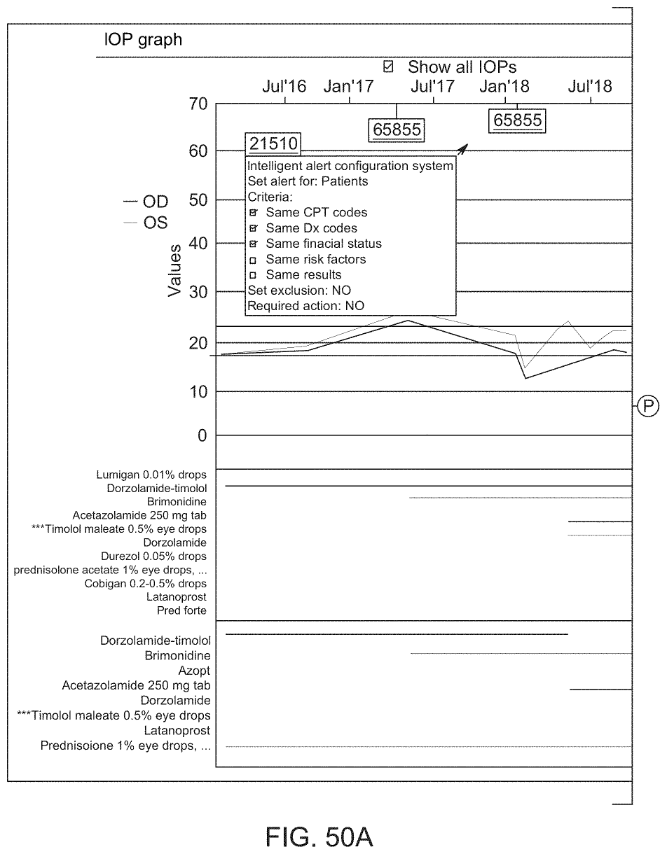

An intelligent alert configuration system, in one embodiment, can be represented in multiple ways, based on several criteria. For example, FIGS. 50A and 50B depict three different representations of an intelligent alert configuration system overlayed upon several different aspects of an application in accordance with an embodiment of the present principles. The alert configuration system of FIGS. 50A and 50B can, in the case of a procedure 21510, display parameters associated with procedures, patients with like procedures, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. In the case of launching the intelligent alert configuration from a result 21520, a different set of parameters can be specified relevant to that result, patients with similar results, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. An example of required actions 21530 denotes several key actions which can occur upon triggering the alert. These include, but are not limited to, a visual or audio alert, tiers of alerts based on values, notifications to be sent and to whom, reminders are to be sent. In the case of launching the intelligent alert configuration from a medication 21540, a different set of parameters can be specified relevant to medications, patients with similar medications, correlations to diagnoses, financial status, risk factors, results, or other relevant criteria, while allowing for exclusion criteria and actions to be taken, while also being able to be assigned to a patient, group of patients, all patients, or a patient or group of patients with specified criteria. An example of exclusions 21550 denotes several key actions which can exclude a patient or patients from an alert.

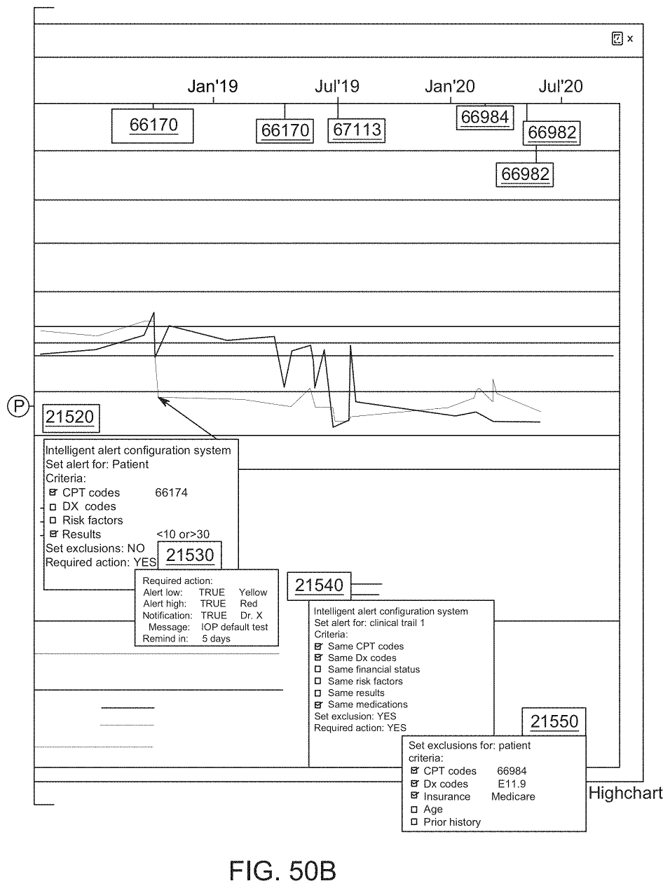

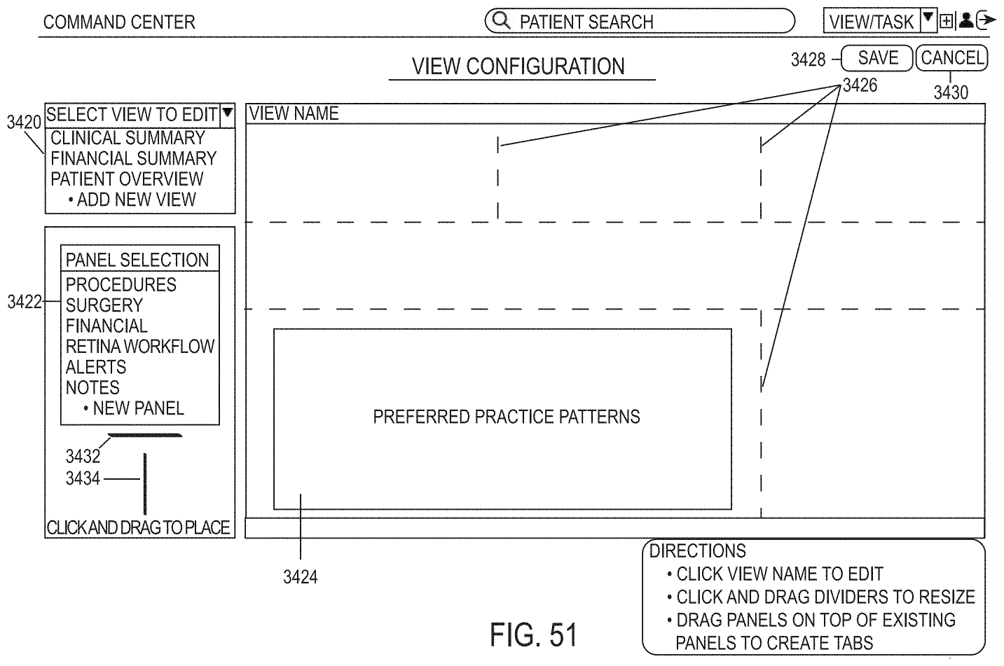

FIG. 51 depicts a view configuration page in accordance with an embodiment of the present principles. The View Configuration page presented in FIG. 51 provides the user a mechanism to create or modify dynamic dashboards to accommodate their unique requirements. These dashboards are referred to herein as Command Center Views. The user identifies the view to be created or modified by selecting the appropriate option from the drop down menu 3420. Once the view has been selected, the user will select a panel to place in the view from the list 3422. Once the panel has been anchored in the View 3424, the dimensions of the panel may be adjusted by means for manipulating the panel frame(s) 3426. When the user is finished creating or modifying the view, the user may save (3428) or cancel (3430) their actions by selecting the associated buttons. Additional horizontal dividers 3432 and vertical dividers 3434 can be dragged and dropped onto the View in order to create new display panels. The dividers also can be removed by clicking on the desired divider and dragging it off the View.

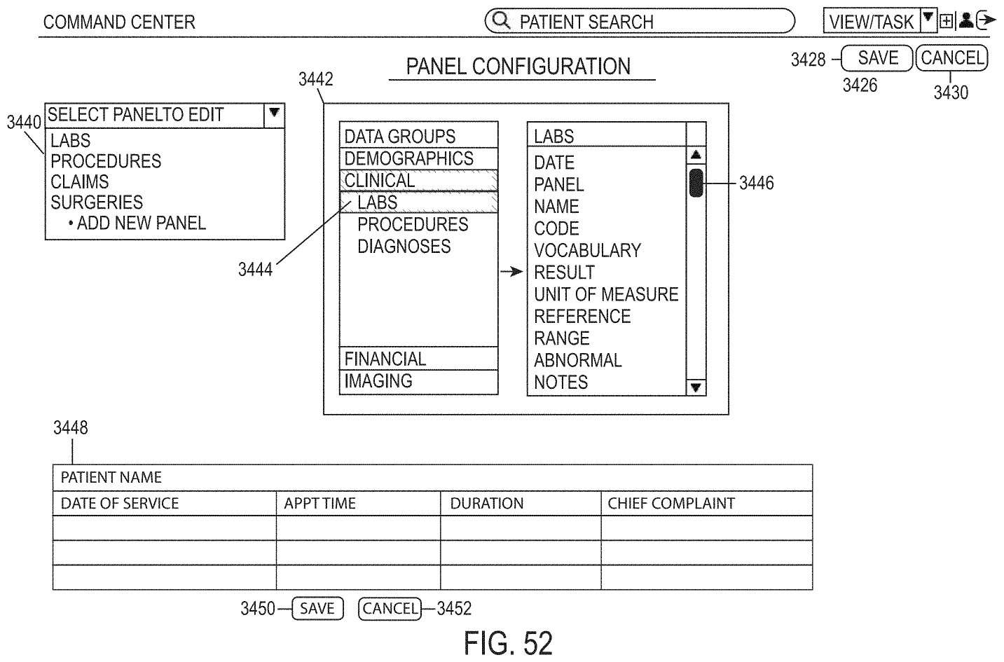

FIG. 52 depicts a view configuration page in accordance with another embodiment of the present principles. The Panel Configuration page presented in FIG. 52 provides the user a mechanism to create or modify display panels to use when populating medical records dashboards of the present principles. The user identifies the panel to be created or modified by selecting the appropriate option from drop down menu 3440. The user then selects data to populate the panel from the Data Selector 3442. Available data 3444 is selected and migrated to the panel's Column List 3446. Users may also add custom fields allowing them to track any data relevant to the user. A preview of the panel 3448 is presented and updated as the user makes changes. When the user is finished creating or modifying the panel, the user may save 3450 or cancel 3452 their actions by clicking the associated buttons. The panels from the drop-down menu 3440 can provide a template for creating a customized Data Command Center in which all information desired by the user can be accessed without leaving the Data Command Center.

In some embodiments, the Data Command Center medical records dashboard can be characterized by an ability to present large volumes of dynamic data in a single display interface whereby the data can be navigated without leaving the screen. For example, the data is either available in a display window, behind a tab, or available via a pop-up window and is thus accessible without leaving the display. In some embodiments, to enable such processing to occur without unacceptable delay in data presentation, and to enable the display panels and flowsheet panels to be reconfigured as described, a Data Command Center architecture provides for flexibility in storage and presentation of dynamic data as well as dynamic caching techniques that allow for prompt presentation. Two-way auto-population techniques can be implemented whereby changes made in the Data Command Center are not only reflected in the display but also the associated electronic medical record is auto-populated as well without leaving the Data Command Center display screen.

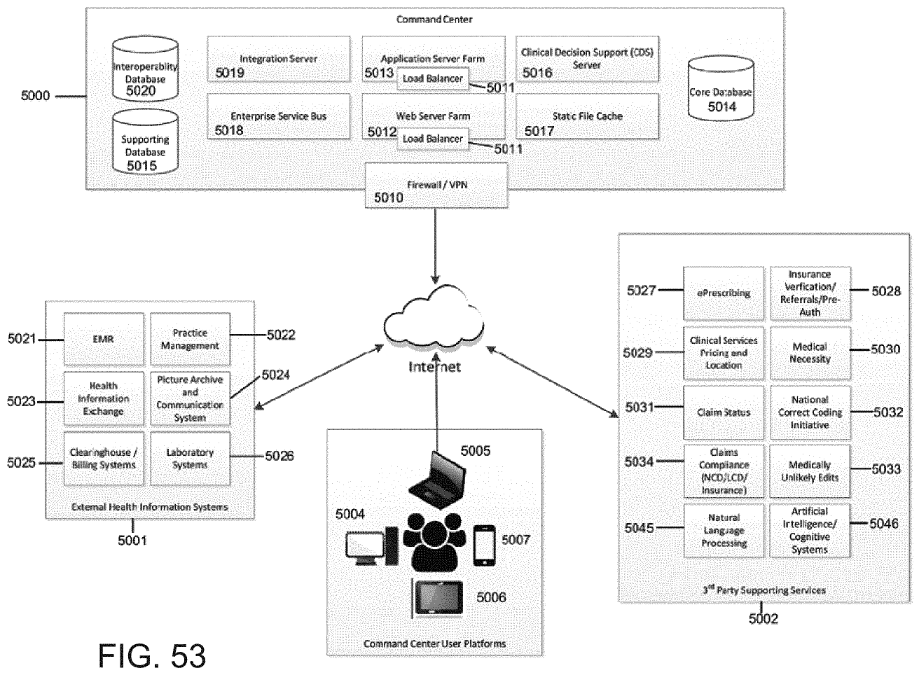

FIG. 53 depicts a Data Command Center architecture 5000 and connectivity to external Health Information Technology systems 5001 and third party services 5002 in accordance with an embodiment of the present principles. The Command Center architecture 5000 is a multi-tenant cloud-based web application. As known by those skilled in the art, multi-tenant means that the application is deployed once and all customers access the same server. Data is segregated by the application so that customers can only access their own data. The Data Command Center is accessible over the Internet via user platforms 5003 that implement a modern web browser (e.g. Internet Explorer 10, Google Chrome) on a desktop computer 5004, laptop 5005, tablet 5006, or a smartphone 5007 with internet connectivity.

The Command Center architecture 5000 illustrated in FIG. 53 is one embodiment of how the system can be configured to support a large user base. For security purposes, access to the cloud platform containing the Command Center is governed by a firewall and Virtual Private Networks (VPNs) 5010. Additionally, end to end encryption is an inherent part of the Command Center architecture 5000 as the Command Center architecture 5000 is optimized to meet the highest privacy and security expectations. Each component allows for both the application of current high strength encryption (ex. AES 256) as well as continuous implementation of evolving data protection and security best practices. The Command Center architecture 5000 configuration supports proven high-availability and disaster recovery practices, including fully encrypted off-site backup and redundant, geographically dispersed operations. Load balancers 5011 distribute the client requests to the most available web server in the web server farm 5012 to optimize response time. The web servers in the web server farm 5012 pass requests through the load balancer 5011 to the application server farm 5013 to continue processing the client requests. The application servers of the application server farm 5013 process the requests and access data from the core database 5014 and supporting database(s) 5015. Depending on the client request, the application servers may interact with the Clinical Decision Support Server 5016 where the primary business logic resides. Once processed, the web server returns the results of the client request back to the client for display. Static files are optimized for retrieval by residing on a dedicated server (static file cache) 5017 where they are cached and optimized for this purpose.

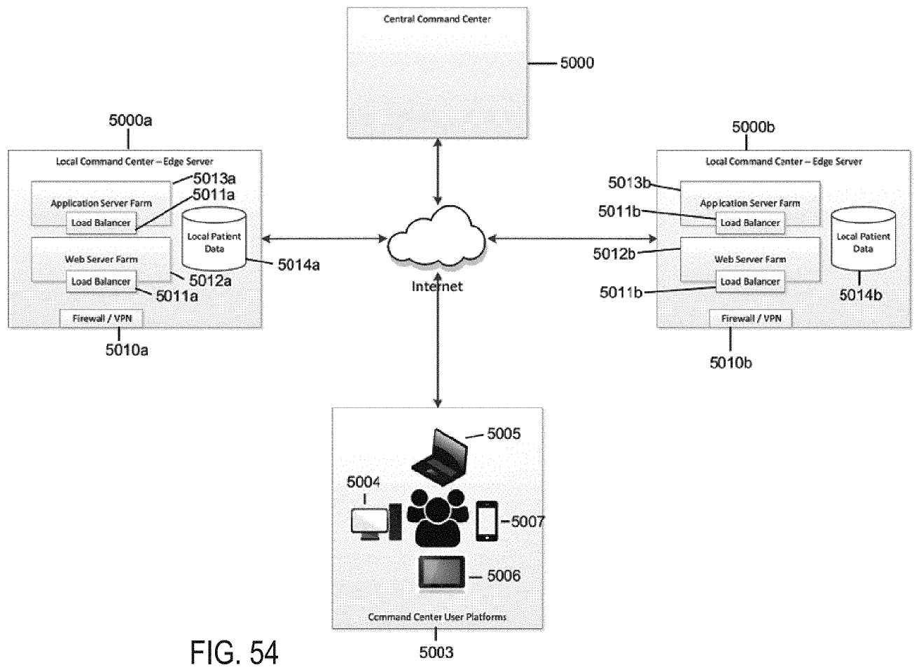

The exchange of information between external health information technology (HIT) systems 5001 and the Command Center 5000 is managed by dedicated servers designed to optimize system throughput and secure communications. All communications take place through a secure VPN 5010 connection. If the integration method is message-based, the sending HIT system 5001 will transmit messages to the Enterprise Service Bus 5018. However, if communication is based on an API standard, the sending HIT system 5001 will communicate with the Integration Server 5019. Both of these servers 5018, 5019 communicate directly with the Interoperability database 5020 and, in turn, with the core and supporting databases 5014, 5015. While communications with third party services 5002 are largely outbound from the Command Center 5000 to the services, it is possible to receive inbound communications. These third party communications will also be handled through the Enterprise Service Bus 5018 and the Integration Server 5019. As illustrated in FIG. 54, examples of HIT systems 5001 include Electronic Medical Records (EMRs) 5021, Practice Management Systems 5022, Health Information Exchanges (HIEs) 5023, Picture archiving and communication systems (PACS) 5024, claims-based systems such as Clearinghouses and Insurance Companies billing systems 5025, and Laboratory Systems 5026.

In exemplary embodiments, many third party supporting services 5002 are integrated to provide feedback and advice. Examples of these services include ePrescribing 5027, Insurance verification including referrals and pre-authorizations 5028, clinical pricing and location services 5029 used to find the best value on purchasing medications, procedures and imaging services, medical necessity checking 5030 to verify a procedure or medication is associated with a correct ICD10 code supporting its use, claim status checking 5031, services in support of the National Correct Coding Initiative 5032, Medically Unlikely Edits 5033 provided by Center of Medicare and Medicaid Services (CMS) to proactively ensure claims are coded correctly to prevent issues in billing, and claims compliance services 5034 which evaluate claims against CMS National Coverage Determination (NCD) and Local Coverage Determination (LCD) guidelines as well as local insurance regulations all in an effort to establish and document medical necessity and to document same in support of streamlined billing. Natural language processing program 5045 and artificial intelligence/cognitive systems 5046 may also be provided to, for example, provide clinical decision support features. In exemplary embodiments, the NCD and LCD guidance is programmed into the Command Center 5000 so that alerts may be generated when a physician attempts to follow a treatment protocol that is non-compliant with the NCD and LCD guidance.

Those skilled in the art will appreciate that data latency may be improved by storing the data in the static file cache 5017 in the server of the distributed network of servers that is closest to the geographic location of the patient's appointment. In an exemplary embodiment, the server closest to the geographic location of the patient's appointment could contain data only for today's patients so that there is less data to query, thus improving the access speed for the data. Also, any data that is not stored locally may be cached locally after it has been accessed for the first time as it is more likely to be accessed a second time, thereby speeding up the data access. This architecture implements Proximity Request storage whereby data accessed most frequently is stored geographically closer to the user to reduce the time it takes to travel over the Internet. This approach is used by Netflix and others when hosting large movie files. In the present case, the most relevant patient data is stored within proximity of where it is stored. Relevant patient data is for patients that have been accessed in the past few days and any patients with an upcoming appointment. Having a smaller local subset of data makes the whole network operate more efficiently.

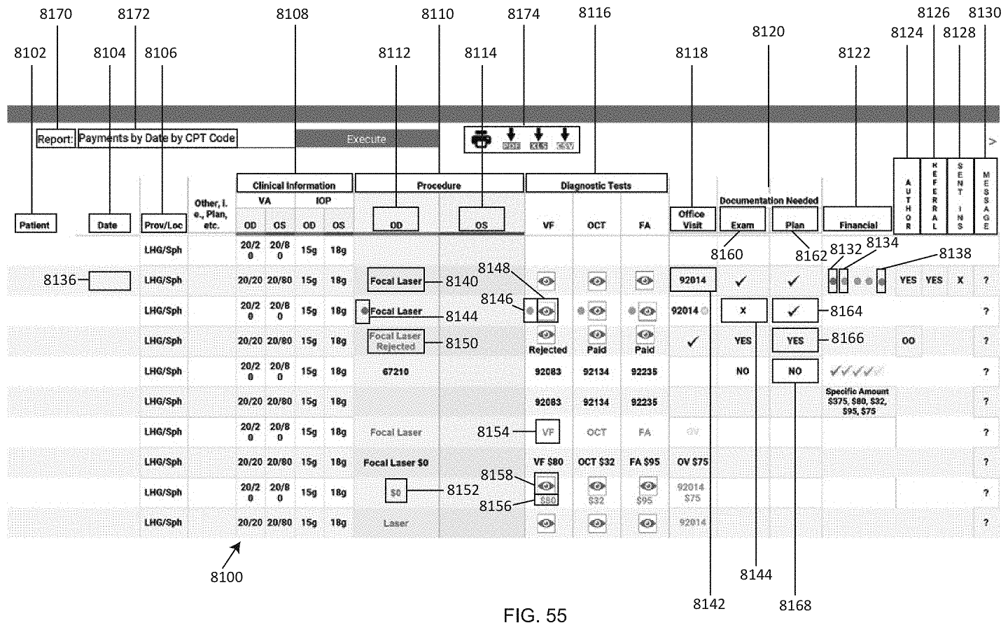

FIG. 55 depicts a medical records dashboard of a Data Command Center of the present principles that enables a healthcare provider while delivering medical care to a patient to participate in revenue cycle management in accordance with an embodiment of the present principles. Without interrupting medical care, the healthcare provider may understand what things cost, whether they have been authorized to do something like a procedure, and whether the previous claims were rejected or paid. Critical compliance issues of properly following the laws can now be often confirmed because if the healthcare provider was paid for something and the services was not performed, the failure to perform the service would be detected. The billers know when charges are paid or rejected and the corresponding diagnosis and CPT codes, but this embodiment correlates the financial with clinical as the doctor is seeing the patient without interrupting the patient flow.

The display 8100 shows a particular patient's name or the name of a number of patients at 8102. The date of a particular encounter is shown at 8104. The patient data can be seen over many encounters listed in the different rows. The provider who delivered the service as well as the location of the provider who saw the patient is provided at 8106. The clinical information that can be displayed simultaneously for the doctors to be able to care for the patient is provided at 8108. This clinical information is different for all medical specialties. Eye doctors might look at vision. Family doctors might look at blood pressure or blood sugar.

A column of procedures 8110 that were performed are defined that need to be measured by specialty. Every specialty might have different procedures and the tool can have multiple columns for different procedures. 8112 and 8114 demonstrate that a procedure can be on different sides of the body. 8112 represents the right (in eye care—OD) side while 8114 represents the left side of the body (in eye care—OS). In orthopedics, procedures of the right knee and left knee could be populated.

8116 provides an example of three types of different diagnostic tests usually performed and billed in an eye doctor's office (e.g., VF, OCT, and FA) but any diagnostic test or other CPT code could be in these columns. 8118 is the office visit that was charged as there are different levels of office visits that a provider can charge at different times. 8120 illustrates whether proper documentation was needed or not needed to be able to bill any of the procedures, diagnostic tests, or office visits.

A financial column 8122 may include an authorization column 8124 where providers as they are seeing patients can actually know whether or not the insurance company has authorized the procedure. A referral column 8126 may indicate whether the proper referral from a family doctor to a specialist was received. A sent insurance column 8128 may be used to indicate whether insurance information has been received for the patient, while message column 8130 may provide messages or tasks that manually or automatically can be created by the user or anyone in the practice to communicate to the user. Financial column 8122 may include many different ways of indicating to a doctor or provider the financial information. For instance, indicator 8132 could be red which could mean rejected, or green for paid, or yellow for partially paid. Another indicator 8134 that is associated with one of the other columns representing something performed and charged by the user that particular date 8136 may also be provided. A third indicator 8138 could be red, yellow or green to represent payments or express costs and corresponding to another of the charges for date 8136. 8140 demonstrates that a focal laser type surgery was performed on a particular encounter date. Indicator 8132 could be associated with the focal laser type surgery 8140 and represents, if red, that the insurance company rejected payment and nothing was paid. Indicator 8134 could relate to whether another item performed on date 8136 was paid. Similarly, indicator 8138 could correlate with whether office visit 8142 was paid.

Instead of all indicators of payment being on one row together in column 8122, the indicators could instead be in the column next to the medical service charged for. In addition, an indicator 8144 may show that there was rejection right next to the procedure, in this case the focal laser, as there is a red dot 8144 which correlates to zero payment. This type of indicator can instead of being all in one column 8122 also can be in each column associated directly with what was done as at indicator 8146 where icon 8148 shows that a VF was performed. Indicator 8150 shows the procedure focal itself can be red or even written ‘rejected’. Similarly, a zero 8152 may be used to show rejected payments. Indicator 8154 demonstrates that a VF was performed but could be highlighted green meaning it was fully paid or red if it was rejected. On the other hand, the amount paid 8156 could in the same cell next to, above or below what was performed, in this case $80.

The healthcare provider may also confirm that something was done or if they want to see the actual item being billed or to interpret it. For example, icon 8158 would allow the healthcare provider to click on it and actually the image itself comes up and an interpretation could be seen. If no image is attached, they might discover that it was not done and billed incorrectly, so the healthcare provider could elect to return the money to the insurance company. This decision may be made rapidly while examining a patient. Additionally, a lack of documentation may be indicated in column 8120. Because doctors often use scribes or assistants, they may have inadvertently not completed the chart. Column 8160 is an exam column, while column 8162 is a plan column. Perhaps they are completed or not, but instantly doctors can see whether a check mark 8164 has been provided to indicate that documentation has been completed. Alternatively, the word ‘yes’ 8166 or any other appropriate word or indicator may indicate that the documentation has been provided, while the word ‘no’ 8168 or other appropriate word or indicator may indicate that the documentation has not been completed. 8144 could be a ‘X’ noting that the indicated documentation was not completed. In this fashion, doctors instantly can be informed and take action to complete the chart.

Doctors may have an interest in running a report on any of the elements, columns or rows to identify payments in one patient or a group of similar patients. 8170 depicts the ability to run a report. The type of report that is requested may be indicated at 8172, where patient list 8102 may list a whole group of patients, perhaps sharing a common insurance company, but doctors can quickly make decisions because they have at their access not only the exact financial information, but in context for each individual patient can understand what was really done that day, how the patient's care was performed, what tests were or were not completed and whether they were paid properly or improperly or, from a compliance point of view not documented correctly as indicated in column 8120. The user may elect to export the data at 8174.

In sample embodiments, the indicators 8132, 8134, or 8138 could be selected to bring up a ledger that matches each CPT code individually with payments. Here the exact row of the date of service with all the CPT codes one or more done that day and payments can all be brought up.

In some embodiments, a method for rules-based data display in a data command center including a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method includes receiving patient-related data from the at least one patient database, comparing the received patient-related data with configuration rules to determine which portions of the received patient-related data are to be displayed in data entry fields of the medical records dashboard, identifying collapsible data entry fields of the at least one collapsible data entry field of the medical records dashboard that are determined to not have any patient-related data to display as collapsed data entry fields, displaying patient-related data in the data entry fields of the medical records dashboard in accordance with the configuration rules and collapsing data entry fields of the medical records dashboard identified as collapsed data entry fields.

In some embodiments, a data command center visual display system that displays data on a display screen includes a computing device comprising at least one processor, a non-transitory computer-readable medium, having stored thereon, software instructions that when executed by the at least one processor of the computing device, cause the computing device to perform operations comprising at least, linking to and receiving patient related medical records including patient data from at least one patient data source, and displaying a medical records dashboard including one or more windows, the medical record dashboard capable of displaying, using the one or more windows, patient data from at least one patient data source including at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, wherein a display of patient data in the medical records dashboard is determined by: comparing the patient data with configuration rules to determine which portions of the patient data are to be displayed in the data entry fields of the medical records dashboard, identifying collapsible data entry fields of the at least one collapsible data entry field of the medical records dashboard that are determined to not have patient data to display as collapsed data entry fields, and displaying patient data in the data entry fields of the medical records dashboard in accordance with the configuration rules and collapsing data entry fields of the medical records dashboard identified as collapsed data entry fields.

In some embodiments, a method for unique patient identification of a subject patient in a data command center including patient-related data received or derived from at least one patient database includes collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning phase.

In some embodiments, a data command center visual display system for determining a unique patient identification includes a computing device comprising at least one processor, a non-transitory computer-readable medium, having stored thereon, software instructions that when executed by the at least one processor of the computing device, cause the computing device to perform operations comprising at least: linking to and receiving patient related medical records including patient data from at least one patient data source, collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning.

In some embodiments, a method for medication management and display in a data command center comprising one or more windows for display and including information received or derived from at least one patient database, the data command center displaying on a screen, using the one or more windows, at least one of medical services, clinical data, examination findings, diagnostic tests, and procedures performed on one or more patients, the one or more windows comprising a plurality of data entry fields for displaying the information received or derived from the at least one patient database, wherein the at least one of the medical services, the clinical data, the examination findings, the diagnostic tests, and the procedures are arranged in on the screen according to at least one of a time and a date that the medical services, the clinical data, the examination findings, the diagnostic tests and the procedures were performed on the one or more patients, includes determining, from at least one of the information received or derived from the at least one patient database and the at least one of the medical services, the clinical data, the examination findings, the diagnostic tests, and the procedures, medications administered to the one or more patients, generating a respective graphical representation for each of the determined medications administered to the one or more patients, and displaying at least one generated, respective graphical representation of at least one medication administered to a patient in the at least one or more windows in context with at least one of the information received or derived from the at least one patient database and the at least one of the medical services, the clinical data, the examination findings, the diagnostic tests, and the procedures, wherein the at least one generated, respective graphical representation of the at least one medication administered to the patient is arranged in on the screen according to at least one of the times and the dates that the at least one medication was being administered to the patient.

In some embodiments, a data command center visual display system that displays data on a display screen includes a computing device comprising at least one processor, a non-transitory computer-readable medium, having stored thereon, software instructions that when executed by the at least one processor of the computing device, cause the computing device to perform operations including at least, linking to and receiving patient related medical records including patient data from at least one patient data source, wherein the patient data includes at least one of medical services, clinical data, examination findings, diagnostic tests, and procedures performed on one or more patients, determining, from at least one of the patient data and the at least one of the medical services, the clinical data, the examination findings, the diagnostic tests, and the procedures, medications administered to the one or more patients, generating a respective graphical representation for each of the determined medications administered to the one or more patients, and displaying using the one or more windows, at least one of medical services, clinical data, examination findings, diagnostic tests, and procedures performed on one or more patients and at least one generated, respective graphical representation of at least one medication administered to a patient in context with at least one of the patient data and the at least one of the medical services, the clinical data, the examination findings, the diagnostic tests, and the procedures, wherein the at least one of the medical services, the clinical data, the examination findings, the diagnostic tests, and the procedures are arranged on the screen according to at least one of a time and a date that the medical services, the clinical data, the examination findings, the diagnostic tests and the procedures were performed on the one or more patients, and wherein the at least one generated, respective graphical representation of the at least one medication administered to the patient is arranged on the screen according to at least one of the times and the dates that the at least one medication was being administered to the patient.

In some embodiments, a method for a display of a graphical representation of complete medical history of a patient in a data command center comprising one or more windows for display and including patient-related data received or derived from at least one patient database, the method includes determining, from the patient-related data, a complete medical history of at least one patient including at least one of medical services, clinical data, examination findings, diagnostic tests, medications administered to and procedures performed on a patient, generating a graphical representation of the determined complete medical history of the patient including the at least one of medical services, clinical data, examination findings, diagnostic tests, medications administered to and procedures performed on the patient, and displaying the generated graphical representation in the at least one or more windows according to at least one of a time and a date that the at least one of the medical services, the clinical data, the examination findings, the diagnostic tests, and the procedures the medical services, the clinical data, the examination findings, the diagnostic tests and the procedures were performed on the one or more patients and at least one of the times and the dates that the medications were being administered to the patient, wherein a user is enabled to select a location in the displayed graphical representation and details regarding the at least one of medical services, clinical data, examination findings, diagnostic tests, medications administered to and procedures performed on the patient related to that selected location are presented to the user.

In some embodiments, the Data Command Center of the present principles, such as the Data Command center 001 of FIG. 1, can provide an in-context image management system configured to display at least one image as a result of a user action in the context of at least one patient's medical data. In accordance with the present principles, one or multiple images can be displayed in the context of one or multiple patients' data. Images can be tagged, interpreted, highlighted, defaulted, prioritized, enhanced, edited, resized, rotated, or otherwise altered and saved or not saved in the images' altered state. In some embodiments, images can be displayed and accessed directly from within rows and columns of patient data. Images can be displayed and accessed directly from thumbnails, either displayed within rows and columns of patient data, or displayed within modules within the context of other modules within the application. Additionally, rows, columns, images, and/or modules can be interacted with through a single interface displaying and enabling the editing of images and/or a patient or multiple patients' data.

In some embodiments of the present principles, in-context display of images and patient related information can begin with a retrieval of patient related information from at least one patient database or server. At least one medical record dashboard can then be displayed including one or more windows for displaying, using a single display interface, patient related information retrieved from or derived from the at least one patient database or server. In some embodiments, the patient related information can include at least one of medical services, clinical data, examination findings, diagnostic tests, and procedures performed on one or more patients. In some embodiments, the one or more windows can include a plurality of data entry fields for displaying the patient related information received or derived from the at least one patient database, such that the at least one of the medical services, the clinical data, the examination findings, the diagnostic tests, and the procedures can be arranged on a display according to at least one of a time and a date that the medical services, the clinical data, the examination findings, the diagnostic tests and the procedures were performed on the one or more patients. In such embodiments, at least one visual representation of at least one image related to the at least one of the medical services, the clinical data, the examination findings, the diagnostic tests, and the procedures performed on the one or more patients can be generated. That is, in some embodiments visual representations of images of at least tests and procedures performed on a patient are generated. In some embodiments, such visual representations can include icons providing links to an available image such that the icons can be displayed in the medical records dashboard and when selected provide links to the available images which can be displayed in a pop-up window concurrently with the displayed patient related information.

Alternatively or in addition, in some embodiments, the visual representations of the images can include thumbnails of the images represented. In such embodiments, a user is able to determine the contents of an underlying image from the displayed thumbnail which assists a user in selecting an appropriate image to view in greater detail or larger scale in, for example, a pop-up window. That is, in accordance with some embodiments, the generated visual representations of available images related to patient care can be displayed in at least one of the plurality of data entry fields, such that when a displayed visual representation is selected, a respective image is displayed concurrently with the patient related information on the display.

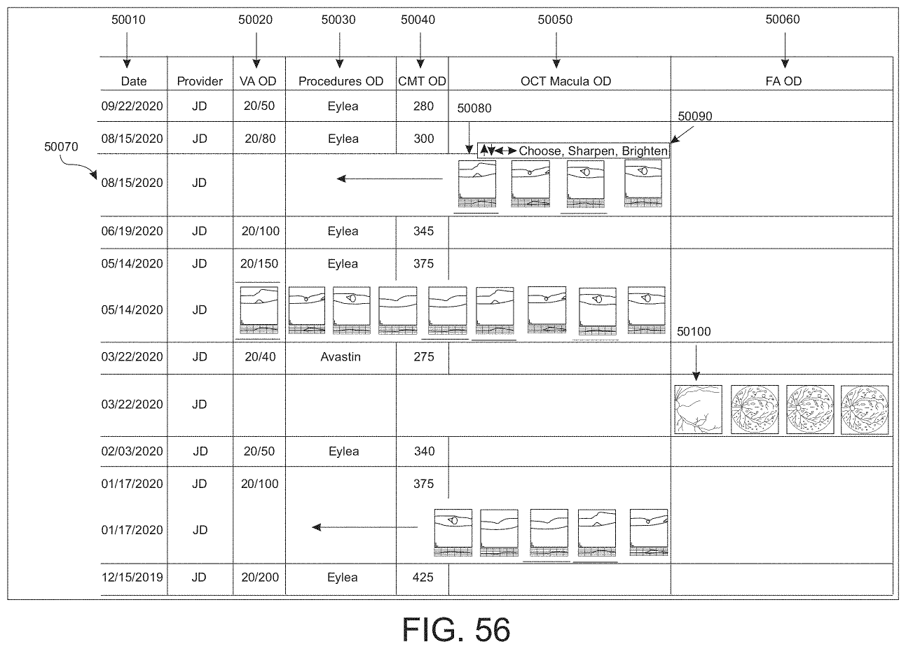

For example, FIG. 56 depicts an embodiment of a display of an in-context image management system displaying thumbnailed images in rows and columns in accordance with an embodiment of the present principles. In the embodiment of FIG. 56, the in-context image management system displays one image within the context of one patient. Patient data, consisting of, but not limited to, date of service, physician, and location 50010, key results 50020, procedures 50030, and additional results 50040, can be displayed alongside thumbnails of images of a single type 50050, or multiple image types 50060. Images can be displayed in rows comprising patient visits on a particular date of service 50070 or on multiple rows for multiple dates of service. Thumbnailed images can consist of one or multiple images for a single date of service 50080 and can enable for interaction with image editing tools to allow the image to be tagged, interpreted, highlighted, defaulted, prioritized, enhanced, edited, resized, rotated, or otherwise altered and saved or not saved in said images' altered state. Multiple image types can be displayed in subsequent columns 50100, and, in some embodiments, the rows and columns can expand to show a larger thumbnail or contract to show a smaller thumbnail. Thumbnails can also be directly accessed and enlarged for more concise review and/or editing.

In the embodiment of FIG. 56, the date of service 50010, provider (not labeled), visual acuity, procedures 50030, and central macular thickness 50040, are displayed alongside an OCT of the macula 50050 and fluorescein angiography 50060, although any combination of data and image can be substituted. In FIG. 56, the date of service 08/15/2020 50070 displays multiple OCT images 50080. The images can be altered through an image editing widget 50090 to flip, resize, sharpen, brighten, or otherwise alter the image or directly accessed for more editing options. For the date of service occurring on 03/22/2020, fluorescein angiography 50100 can be viewed alongside other images. In some embodiments, images of any displayable type can be accessed individually or in groups. When the existence of multiple images of the same type occurs on the same date of service, images can be configured to be displayed in a predetermined or selected order or priority.

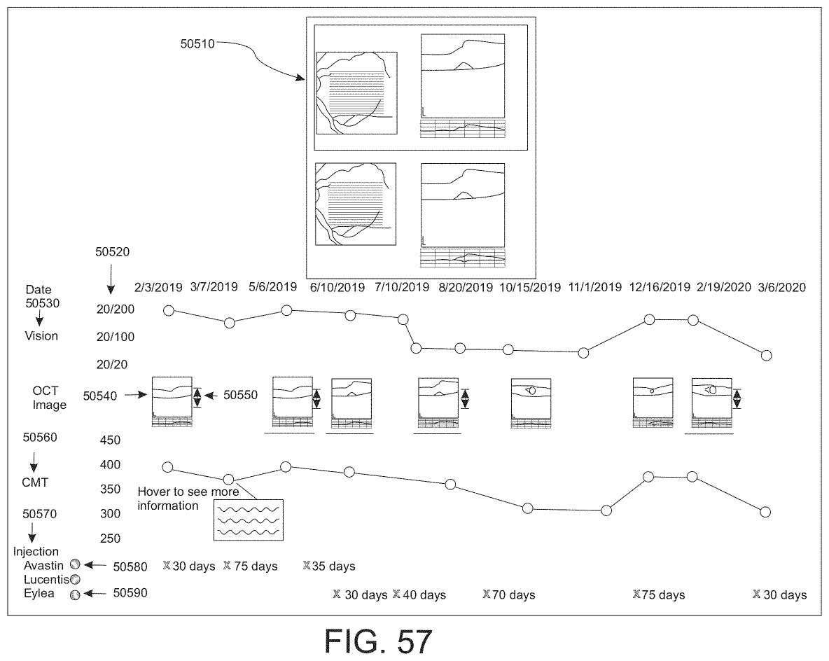

FIG. 57 depicts another embodiment of a display of an in-context image management system displaying images in the context of graphically visualized medical data modules in accordance with an embodiment of the present principles. In the in-context image management system of FIG. 57, 50510 can be displayed within the context of graphical representations of patient data. Large or thumbnailed versions of images can be display and edited and/or otherwise altered. In FIG. 57, within the image viewing module 50510 one or multiple images can be interacted with at the same time, not limiting the user to accessing and editing a single image before moving on to the next.

In the embodiment of FIG. 57, date of service 50520 is displayed horizontally instead of vertically. Those skilled in the art will appreciate that relevant axes may be horizontal, vertical, along a z-axis, or any other axis as deemed appropriate. In the embodiment of FIG. 57, visual acuity 50530 is now displayed horizontally and graphically. Those skilled in the art will appreciate that the representation of underlying data can be displayed as text, lists of text, formatted or unformatted text, as a graphical symbol or icon, plotted on a graph, or otherwise graphically represented. OCT diagnostic images 50540 can be displayed horizontally within the context of relevant data. OCT diagnostic images can also be tagged 50550 for ease of assessment in such a manner as to enable a medical care provider to later save the step of actually viewing a detailed version of the image by denoting if the image was an improvement or deterioration from the previous study. In some embodiments, algorithms can be implemented to automatically denote an increase or decrease in key results returned within image metadata or by other digital means. Additional results, such as central macular thickness 50560, can also be displayed horizontally in accordance with some embodiments. Key results, correlated against events, actions, and diagnostic imaging is an example of in-context image management. In some embodiments, key procedures 50570 can also display alongside images and results. Procedures, results, images, and other patient data may trigger rules to highlight, flag, or otherwise draw attention to their relevant impact on the study or compliance. In the embodiment of FIG. 57, an Avastin injection 50580 is highlighted, for example, in red to denote an issue with compliance, impact on relevant results, or other problem or deficiency related to the injection. In the embodiment of FIG. 57, an Eylea injection 50590 is highlighted, for example, in green to denote proper compliance, and improvement in results, or other improvement or positive result related to the injection. Key indicators expressed throughout the in-context image management system offer caregivers quick access to relevant information while maintaining the ability to view and edit images.

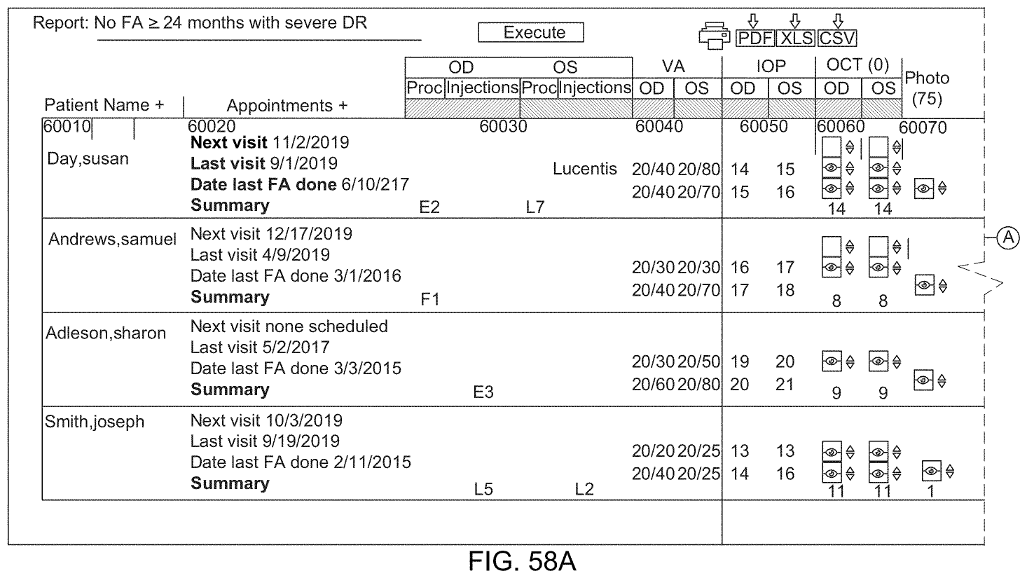

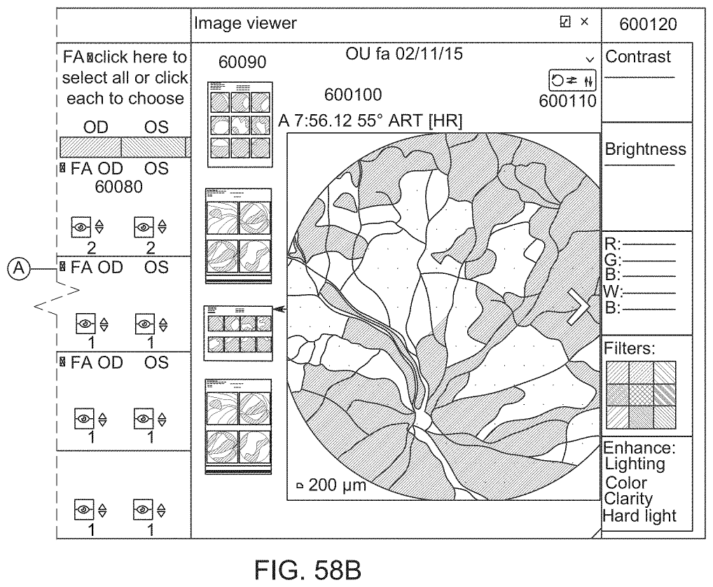

FIGS. 58A and 58B depict an embodiment of a display of an in-context image management system displaying multiple patients and multiple images in accordance with an embodiment of the present principles. In the embodiment of FIGS. 58A and 58B depict a column denoting individual patients 60010 lists one or more patients with relevant patient data in subsequent columns. A summary of patient information is displayed within a column 60020, and/or individual columns can display precise patient data such as procedures and injections 60030, visual acuity 60040, and intraocular pressure 60050. In the embodiment of FIGS. 58A and 58B depict, patient data is displayed alongside diagnostic images such as an OCT 60060, fundus photos 60070, and fluorescein angiography 60080. In the embodiment of FIGS. 58A and 58B depict, an icon or graphical representation depicts the presence of an image. One skilled in the art will appreciate that an icon or graphical representation, such as displayed in columns 60060, 60070, and 60080, can be substituted for a thumbnail as denoted in 50080 of FIG. 56.

In some embodiments, a series of thumbnailed images can be displayed in a column or row 60090 adjacent to a full-sized or enlarged image 60100. Such images can be configured to default or prioritize in a specified order. In some embodiments, images can be edited using buttons, icons, or other graphical interfaces to enable editing such as resizing, rotating, and/or flipping horizontally or vertically 60110, as well as more in depth editing tools 60120 such as adjusting contrast, brightness, individual color channels, applying filters, or individual attribute enhancements.

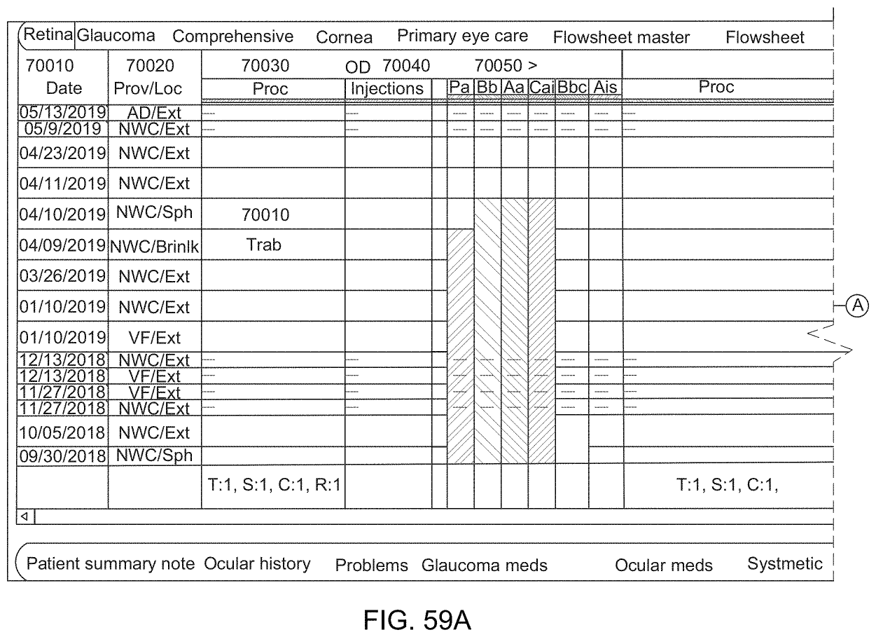

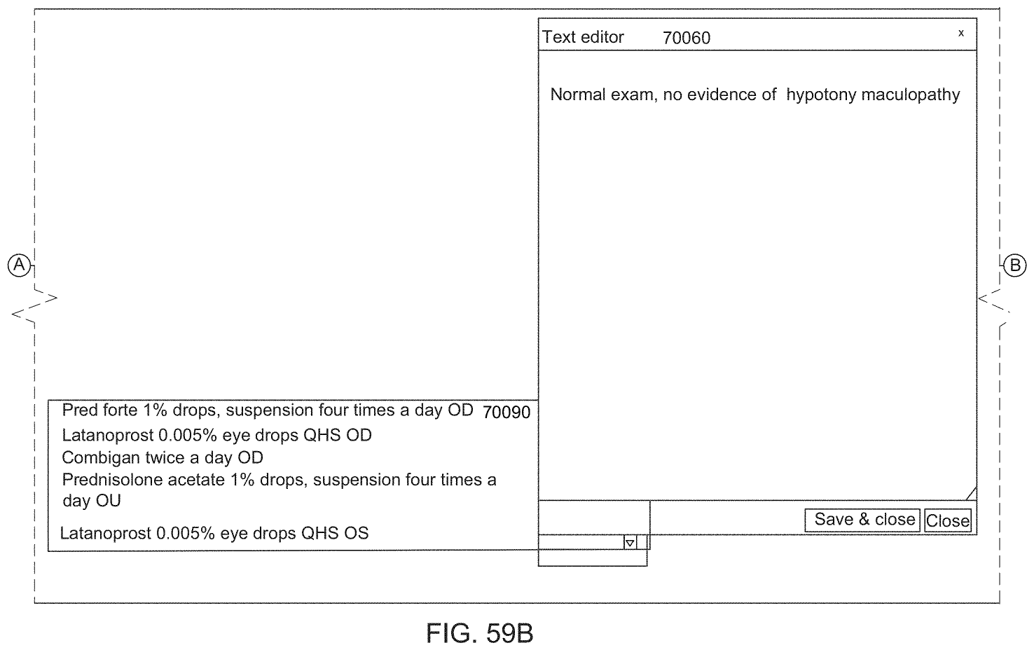

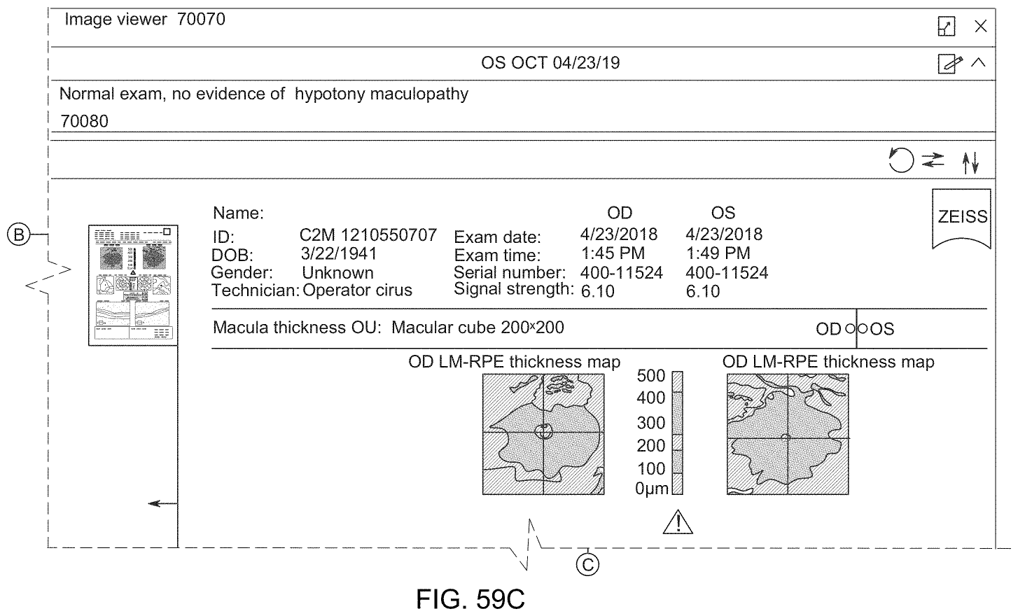

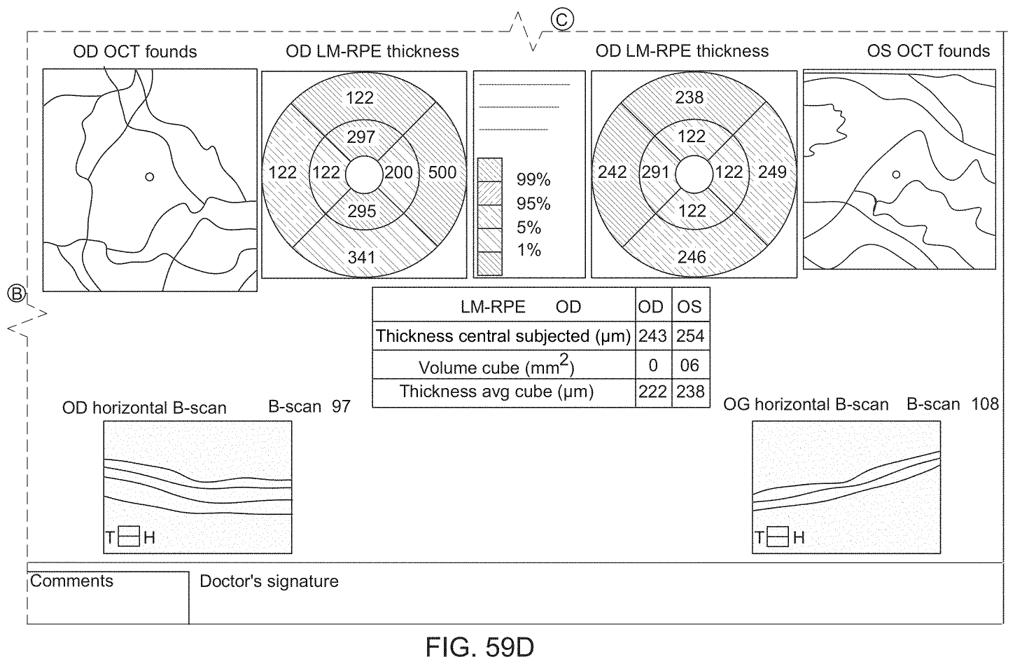

FIGS. 59A, 59B, 59C, and 59D depict an embodiment of a display of an in-context image management system enabling a direct edit of image information in accordance with an embodiment of the present principles. In the embodiment of FIGS. 59A, 59B, 59C, and 59D, date of service 70010, provider and location 70020, procedures 70030, injections 70040, and medications 70050 illustratively comprise patient data. In FIGS. 59A, 59B, 59C, and 59D, a text editor 70060 is launched in context of both patient data and relevant imagery 70070. Edited text inserted by a user can update and/or overwrite existing interpretation text 70080 upon completion, by, for example, selecting Save and/or Close. In some embodiments, images can be selected using icons or thumbnails present in any row or column indicating that an image is present. In some embodiments, the icons and/or thumbnails can include relevant information about the image to enable rapid identification of which image or images a user wishes to select. As depicted in FIGS. 59A, 59B, 59C, and 59D, additional modules can be selected and edited while viewing the image through the in-context image management system 70070, such as recording a patient summary note 70090. Those skilled in the art will appreciate that any available module which allows for editing may be substituted for patient summary note in this example. Embodiments of the present principles enable the editing of patient data while viewing the image in-context, and conversely the editing of any image while viewing patient data in-context.

In accordance with the present principles, a user/medical care provider is enabled, by an in-context image management system of the present principles, to view/manipulate/edit images in context of patient data via an ability to select icons/thumbnails of images, for viewing/manipulating/editing of images while having access to (i.e., viewing) patient data. In some embodiments of the present principles, portions of images and/or image viewers can be transparent such that underlying patient data can still be viewable/selectable while viewing an image.

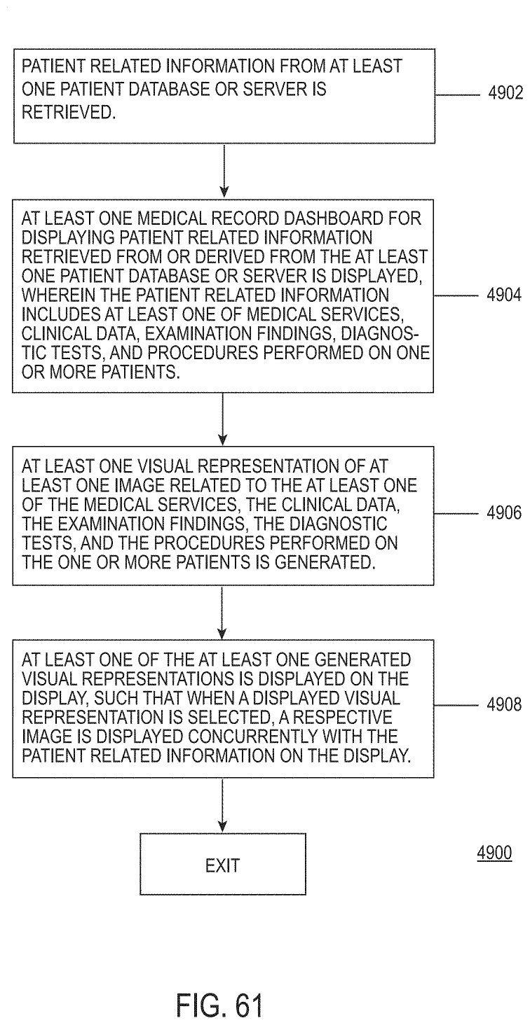

FIG. 61 depicts a flow diagram of a method 4900 for in-context display of images and patient related information in accordance with an embodiment of the present principles. The method 4900 can begin at 4902 during which patient related information from at least one patient database or server is retrieved. The method 4900 can proceed to 4904.

At 4904, at least one medical record dashboard including one or more windows for displaying, using a single display interface, patient related information retrieved from or derived from the at least one patient database or server including at least one of medical services, clinical data, examination findings, diagnostic tests, and procedures performed on one or more patients can be displayed. In some embodiment, the one or more windows can include a plurality of data entry fields for displaying the patient related information received or derived from the at least one patient database, wherein the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. The method 4900 can proceed to 4906.

At 4906, at least one visual representation of at least one image related to the at least one of the medical services, the clinical data, the examination findings, the diagnostic tests, and the procedures performed on the one or more patients is generated. The method 4900 can proceed to 4908.

At 4908, at least one of the at least one generated visual representations is displayed on the display in at least one of the plurality of data entry fields, such that when a displayed visual representation is selected, a respective image is displayed concurrently with the patient related information on the display. The method 4900 can then be exited.

In some embodiments, a visual representation of a patient-related image in accordance with the present principles can include at least one thumbnail representation of an image.

In some embodiments, a user of an in-context image management system in accordance with the present principles is able to select an image to view by reviewing the thumbnail representations of the available patient-related images.

In some embodiments, a selection of a displayed visual representation of patient-related images can include clicking on the displayed visual representation of patient-related images using a pointing device.

In some embodiments, a selection of a displayed visual representation of patient-related images can include hovering over a displayed visual representation of patient-related images using a pointing device.

In some embodiment of the present principles, a system for in-context display of images and patient related information include a computing device comprising at least one processor and a non-transitory computer-readable medium, having stored thereon, software instructions. In such embodiment, when the software instructions are executed by the at least one processor of the computing device, the system is configured to perform operations including at least retrieving patient related information from at least one patient database or server, displaying at least one medical record dashboard including one or more windows for displaying, using a single display interface, patient related information retrieved from or derived from the at least one patient database or server including at least one of medical services, clinical data, examination findings, diagnostic tests, and procedures performed on one or more patients, where the one or more windows include a plurality of data entry fields for displaying the patient related information received or derived from the at least one patient database, and where the at least one of the medical services, the clinical data, the examination findings, the diagnostic tests, and the procedures are arranged on a display according to at least one of a time and a date that the medical services, the clinical data, the examination findings, the diagnostic tests and the procedures were performed on the one or more patients. In such embodiments the system is further configured to perform operations including generating at least one visual representation of at least one image related to the at least one of the medical services, the clinical data, the examination findings, the diagnostic tests, and the procedures performed on the one or more patients, and displaying at least one of the at least one generated graphical representation on the display in at least one of the plurality of data entry fields, such that when a displayed graphical representation is selected, a respective image is displayed concurrently with the patient related information on the display.

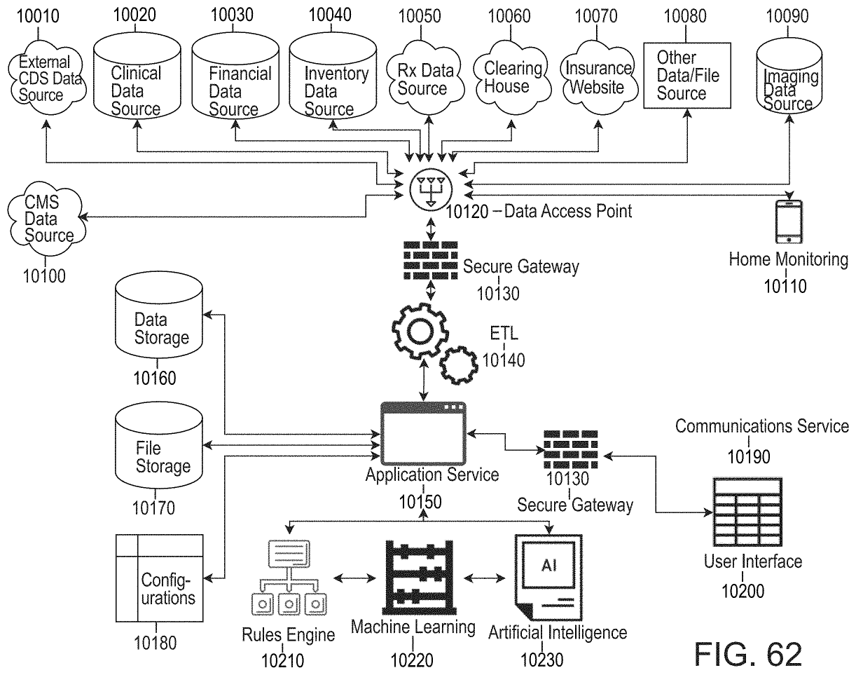

FIG. 62 depicts an embodiment of a Data Command Center architecture in accordance with an alternate embodiment of the present principles. FIG. 62 further depicts how the Data Command Center connects to external Health Information Technology systems and third party services 10010-10110. The Data Command Center architecture of FIG. 62 is a multi-tenant cloud-based web application. Multi-tenant infers that the application is deployed once, and all customers access the same server or farm. In the embodiment of FIG. 62, data is segregated by the application so that customers can only access their own data. The Data Command Center is accessible over the Internet via a user interface 10200 through a Secure Gateway 10130.

The Data Command Center architecture illustrated in FIG. 62 is one embodiment of how the system can be configured to access data from multiple sources, compile and evaluate the data, take action, and display the data to the end user. In FIG. 62, for security purposes, access to the cloud platform containing the Data Command Center is governed by a Secure Gateway 10130, whether a connection exists between the Data Command Center and the end user or the Data Command Center and external data sources. Additionally, end to end encryption can be an inherent part of the Data Command Center architecture of FIG. 62 as the Data Command Center architecture is optimized to meet the highest privacy and security expectations. Each component allows for both the application of current high strength encryption (ex. AES 256) as well as continuous implementation of evolving data protection and security best practices.

In the embodiment of FIG. 62, the exchange of information between multiple external health information technology (HIT) systems 10010-10110 and the Application Service 10150 is routed through an external Data Access Point 10120. Exchange of data may occur in a monodirectional or bidirectional manner, dependent upon access and scenario. In such embodiments, external data sources can include, but are not limited to:

External CDS Data Sources: External Clinical Decision Support Data Sources 10010 may include Registries, Societies, Industry Resources, Insurance Companies, and other sources make available relevant rules and data to evaluate against.

Clinical Data Sources: Clinical Data Sources 10020 may include EHR/EHRs, available anonymized Clinical Data Sources, Referral Management Data Sources, and other available clinical data repositories. Clinical data may be read and/or written back to the data source.

Financial Data Sources: Financial Data Sources 10030 may include banks, Clearing Houses, Insurance Data Sources, Center for Medicare and Medicaid Services Data Sources, and other repositories of financial data. Financial Data may be read and/or written back to the data source.

Inventory Data Sources: Inventory Data Sources 10040 may include internal and/or external Inventory Management Systems. Inventory Data may be read and/or written back to the data source.

Rx Data Sources: Rx Data Sources 10050 may include available e-Prescribing Data Sources, Pharmacy Data Sources, and other external Prescription Data Sources. Rx Data may be read and/or written back to the data source.

Clearing House: Clearing Houses 10060 contain large amounts of Financial and Insurance Data, access to Insurance Data Sources, and Claims Scrubbing Mechanisms. Clearing House data may be read and/or written back to the data source.

Insurance Websites: Insurance Websites 10070 offer direct interaction with Insurance Data Sources beyond standardized Clearing House access. Insurance Website data may be read and/or written back to the data source.

Other Data/File Sources: A key benefit of the Command Center is the ability to pull relevant data from various, disparate data sources. Other Data/File Sources 10080 may include any repository of relevant data, Clinical, Insurance, Financial, CDS, or otherwise. Other data sources may comprise non-standard data repositories, smartphone apps, or even a Google or Excel spreadsheet that a practice records relevant data in. Other Data/File Sources may be read and/or written back to the data source.

Imaging Data Source: Imaging Data Sources 10090 may consist of locally hosted image repositories, diagnostic testing systems, cloud-based image repositories, or other sources of medically relevant imaging.

CMS Data Source: The Center for Medicare and Medicaid Services (CMS) 10100 make available patient data for Medicare and Medicaid patients for research and development.

Home Monitoring Devices: With the advent of the IoT, more and more home-based monitoring devices 10110 are being used to track important health information from a patient's home. Such information may be relevant to patient care, and as such, the Command Center reads, compiles, and evaluates said data.

In the embodiment of the Data Command Center of FIG. 62, data can be posted and/or retrieved from External Data Sources 10010-10110, routed through a Data Access Point 10120, validated by a Secure Gateway 10130, and then may be processed through an ETL (Extract Transform Load) service 10140. The ETL service 10140 is utilized by the Data Command Center to perform necessary transformation of data from proprietary or non-standard formats into industry standard formats that are universal, manageable, and portable. It should be appreciated that an inverse ETL process can be utilized to take previously transformed data and return said data in its initial format.

Utilizing such a technique, no one data source need be solely responsible for any given data point. Where accessible, data which can be missing from one system can be imported from a different system if such data exists. Employing the same logic, missing data which may not be found elsewhere can be flagged as missing to inform a user such data is not present in any relevant data source.

A service or group of services, referred to as the Application Service 10150, manage the routing of data between storage 10160-10170, rules engine 10210-10230 services, configuration services 10180, and the user interface 10200 and/or communications service 10190. The Application Service 10150 is responsible for the overall management of data and other services. Data, after transformed into industry standard formats, can be stored in a Data Storage repository 10160, in one or multiple formats dependent upon use case. Data may also not be stored, and directly posted to the User Interface 10200 through a Secure Gateway 10130. Data can also be converted to files and stored within the File Storage repository 10170. Files received can be stored in the File Storage repository 10170, can be reformatted into industry standard formats by the ETL service 10140 and stored in the File Storage repository 10170, or can be transformed into data and stored in the Data Storage repository 10160. Files may also not be stored and directed posted to the User Interface 10200 through a Secure Gateway 10130. Data and Files, stored or not stored, can be posted to the User Interface 10200 through a Secure Gateway 10130 with additional information and/or edits/enhancements/augmentation to their content.

Data and Files can, dependent upon use case, be processed through a Rules Engine 10210 responsible for evaluating a set of rules against the Data and Files. Rules can be predefined, retrieved from external data sources 10010-10110, generated at runtime, and/or generated based on Machine Learning 10220 and/or Artificial Intelligence 10230. Machine Learning 10220 can employ a number of techniques, to adapt to new information, and Artificial Intelligence 10230 may compile, coordinate, and evaluate data for trends to further define new rules.

All relevant data can then be processed through the Application Service 10150 and can be returned to the User Interface 10200 through a Secure Gateway 10130, ensuring proper security and encryption is in place to protect sensitive information. The Configurations service 10180 can store predefined lists used to determine which data can be displayed and can work alone or in conjunction with the Rules services 10210-10230 to make the determinations. The Rules services 10210-10230 can also utilize the Application Service 10150 to access the Communications service 10190 for external automated communications, where appropriate. It should be appreciated that best practices, and evolving technology, can be used to determine the best approach for data retrieval, transformation, storage, and transmittal in accordance with the present principles.

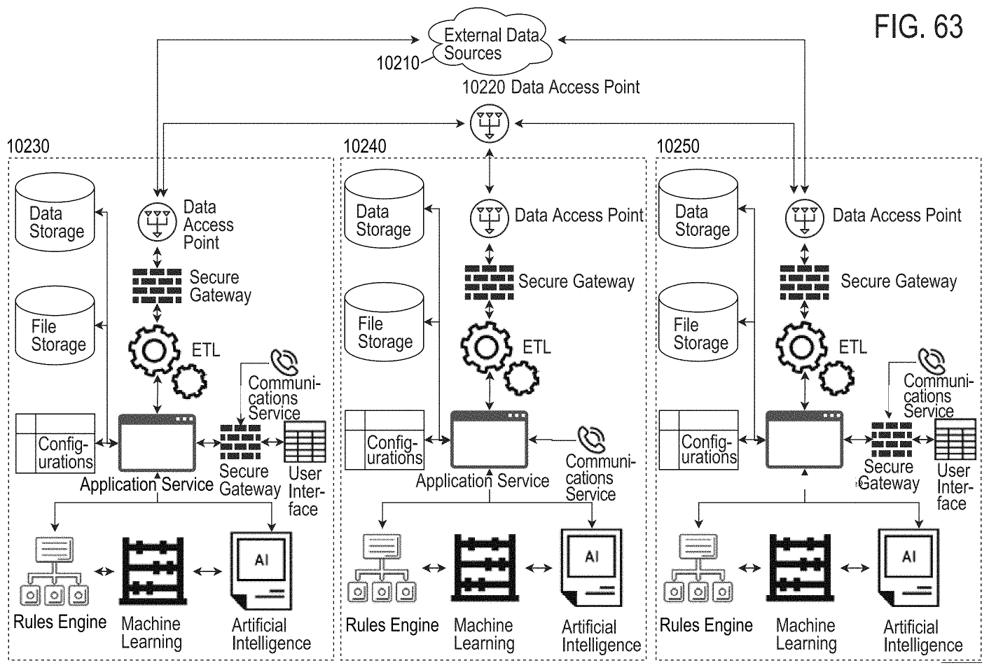

FIG. 63 depicts an implementation diagram of the ability of the Data Command Center of the present principles to connect several instances of the application to each other, as well as to external data sources, such that all can function as a single, logical application. As depicted in FIG. 63, utilizing a multi-tenant architecture, all data can be centralized, and managed by the Application service 10150 of, for example, FIG. 62. Logical separation of data occurs at the software level. That is, FIG. 63 is an example of an implementation diagram whereby multiple tenants 10230, 10240 can subscribe to multiple external data sources 10210, which can consist of both unique and shared resources. In this example, the first Client 10230 and second Client 10240 can exist within a single server or farm, yet the logical separation of data still exists. A third instance of the application 10240 can be used to connect the Clients to each other through use of an external or internal Data Access Point 10220. As such, each Client functions as its own entity, yet sharing of data between clients can be achieved.

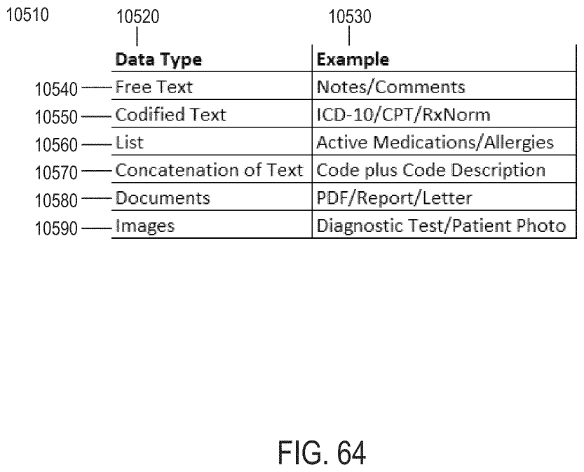

FIG. 64 depicts a Table listing of examples of different data types able to be accessed by a Data Command Center of the present principles such as the Data Command Center of FIG. 62. Data takes many forms, and is an overarching term for information, in general, regardless of format. In the case of Healthcare IT, certain standards exist, and data types which are common to the development of digital solutions. For the purpose of this document, several examples 10510 of data being accessed are listed below.

These data are listed by type 10520 and Example 10530. For example, such data can include but is not limited to:

Free Text 10540: Generally, any text that is entered free-hand without pertaining to a selectable list of options or industry standard. Notes and Comments fall into this category.

Codified Text 10550: Certain text within Healthcare IT applications are directly connected to industry standard code lists, such as ICD-10, CPT Codes, and RxNorm codes.

List 10560: Data elements can be comprised of lists of Problems, Medications, Allergies, or the like, and are stored and transported as such.

Concatenation of Text 10570: Not all data is maintained in normalized and standardized formats. As such, the ability to read and parse sections of text is important. In the case of data strings which can contain several elements, such as a code and a description, or a date and a doctor or location, certain elements can be important, while others can be ignored.

Documents 10580: Some data is simply stored in a precompiled format, such as a PDF or Word document. As such, the document can be stored and transported or can be parsed and data elements pulled out to populate discrete data fields.

Images 10590: Images can contain nothing more than the image, itself, and as such can be stored and transported in native format or converted to a different format. Images can also contain metadata and/or data elements within the image. As such, they can be parted, and data elements pulled out to populate discrete data fields.

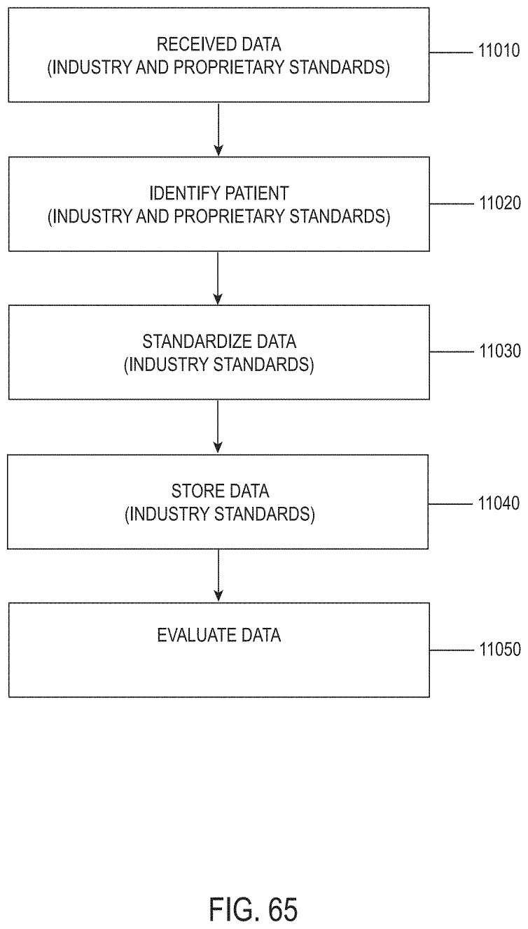

FIG. 65 depicts a high-level workflow diagram of the receipt, storage, and evaluation of data associated with a Data Command Center of the present principles. In the embodiment of FIG. 65, the Data Command Center can use constant poling at interval or monitoring for change to determine when new data is available. When data is received from an external system 10010-10100 through any of the messaging standards or API methods, a standard workflow can be followed as depicted in the embodiment of FIG. 65. The first step (11010) is to receive data using the previously discussed methods. The message is parsed and the patient identifying information is extracted so the correct Patient can be identified (11020). Once the patient is identified, the data is standardized (11030) so that the data can be effectively used in the Data Command Center and then can be stored (11040). The last step in the process is to evaluate the data (11050) using a variety of mechanisms using advanced analytics as will be described in greater detail below.

In accordance with the present principles, there exist multiple criteria which affect the display and augmentation of data fields, considered by the inventors as Dynamic Data Fields, for a Data Command Center of the present principles. In some embodiments, the Data Command Center enables the medical records dashboard to intelligently alert by any means, gray out, expand, collapse, display, and/or hide columns, rows, fields, and/or any other portion of the medical records dashboard to show precisely what a user wishes to display, or can alert by any means, gray out, expand, collapse, display, and/or hide columns, rows, fields, and/or any other portion of the medical records dashboard based on rules or triggers overriding the user's pre-determined display to show important details which the user should be made aware of. For example, in one embodiment, a Flowsheet including patient treatment and health information can be accessed from an EHR system using, in some embodiments, an icon/button, keystroke, or series of keystrokes, gesture, voice command, or other means, associated with at least one of the Data Command Center and the medical records dashboard. Upon accessing the Flowsheet, a set of Rules and Configurations associated with, for example, a Rules Engine, for example the Rules Engine 10180 of FIG. 62, can be evaluated to determine which data from the Flowsheet is to be displayed in a medical records dashboard of the present principles. For example, in some embodiments, the Rules Engine 10180 of FIG. 62 can include information on what data to display, and in turn what portions of the medical records dashboard to display, based on, including but not limited to, at least one of an identity of a medical care provider, an identity of a patient, a patient's medical history, a medical care provider's specialty, a user's custom configurations, patient appointment type, conditions of a patient, patient procedures, risk factors, diagnostic tests and/or results, future orders, future appointments, co-management status, predefined triggers, triggers generated in real time, values recorded, values not recorded, calculated values, and absolute values for display.

For example, in some embodiments in accordance with the present principles, Rules and Configurations can be predetermined and stored, for example, in the Rules Engine 10180 of FIG. 62, for determining which data of a Flowsheet and, as such, which portions of the medical records dashboard to alert by any means, gray out, expand, collapse, display, and/or hide based on known rules. Alternatively, or in addition, in some embodiments, a user can self-configure the medical records dashboard to display only certain portions or to hide certain data of a Flowsheet and, as such, which portions of the medical records dashboard to display or to hide using, for example, a user interface associated with the medical records dashboard. Alternatively, or in addition, data of the Flowsheet can contain an indicator (e.g., a flag) that can be identified by, for example, the Rules Engine 10180 of FIG. 62, for determining when and if a piece of data should be displayed, or can alert by any means, gray out, expand, collapse, display, and/or hide.

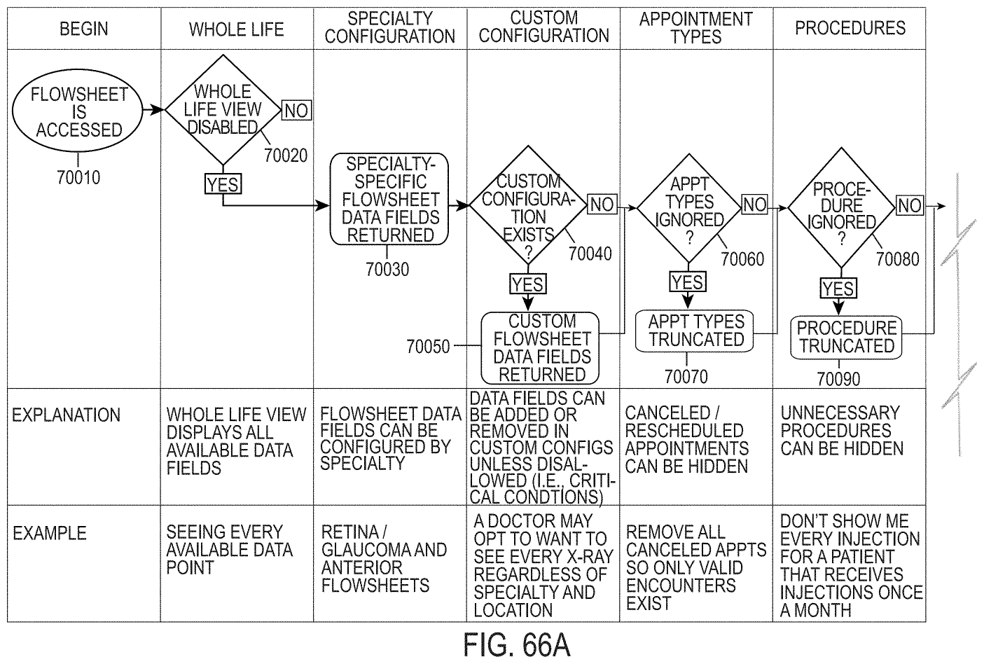

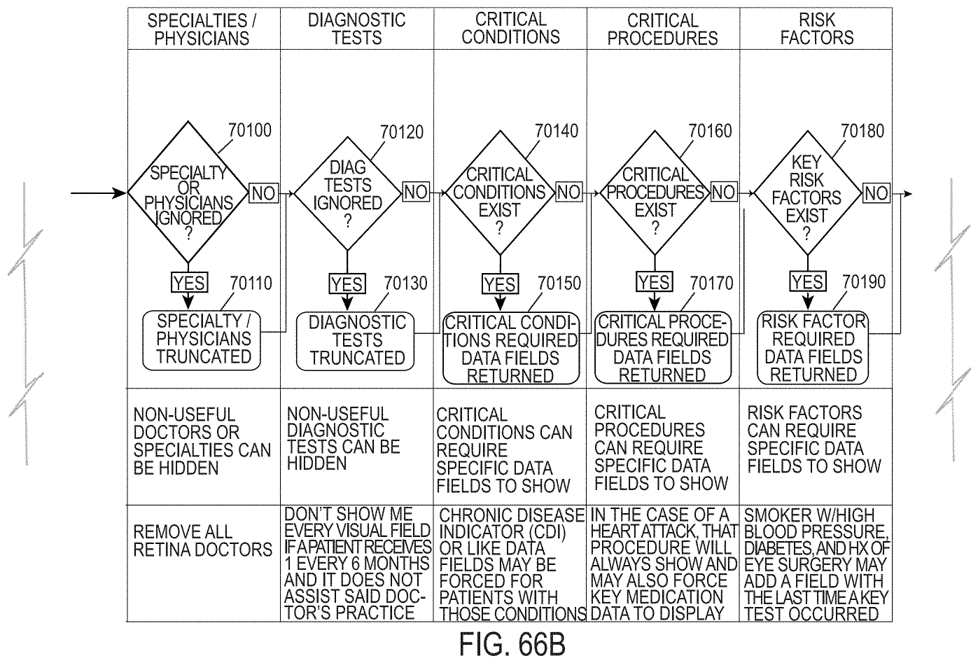

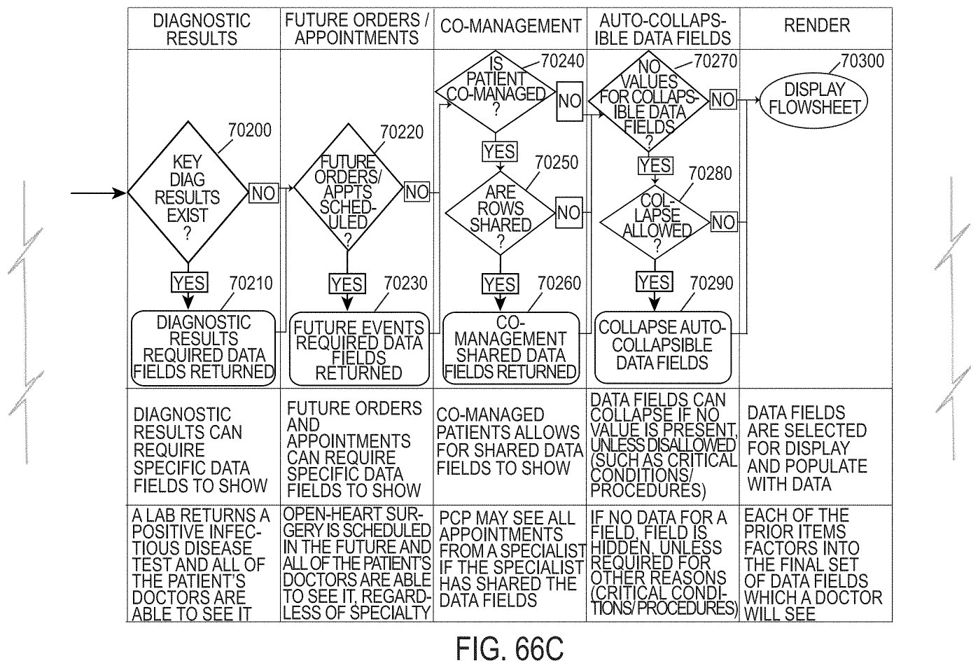

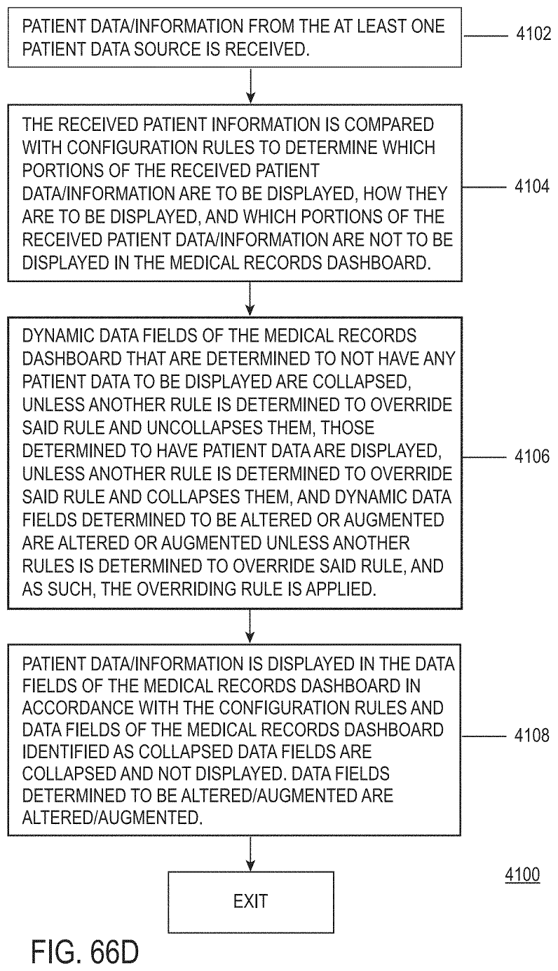

FIGS. 66A, 66B, and 66C, collectively referred to as FIG. 66 herein, depict a workflow diagram of an alternate embodiment of a process for intelligently alerting by any means, graying out, expanding, collapsing, displaying, and/or hiding columns, rows, fields, and/or any other portion of the medical records dashboard based on rules. In the embodiment depicted in FIG. 66, the process begins at 70010 during which a Flowsheet including patient treatment and health information is accessed from, for example, an EHR system. The process illustratively proceeds to 70020. At 70020, it is determined if, what the inventors refer to as a “Whole Life View”, is disabled. More specifically, at 70020 it is determined if all the data in the Flowsheet should be displayed in the medical records dashboard. If Whole Life View is disabled, the process proceeds to 70240 during which all of the data from the Flowsheet is displayed in the medical records dashboard. If not, the process illustratively proceeds to 70030.

At 70030, it is determined if at least one Specialty Configuration exists. For example, in some embodiments a Specialty Configuration can include a configuration based on the specialty of a medical care provider. If so, the process proceeds through 70030 during which all Specialty Configurations are identified such that the data from the Flowsheet can be filtered to only display data associated with identified Specialty Configurations. For example, as previously described, in some embodiments, information associated with medical care provider specialties and data to be displayed and hidden in the medical records dashboard dependent on the specialties can be predetermined and stored in the Rules Engine 10180 of FIG. 62. In accordance with the present principles, Specialty Configurations can require certain portions, columns, and/or rows of the medical records dashboard to be displayed or hidden. After the Specialty Configurations are identified and/or if it is determined that a Specialty Configuration does not exist, the process illustratively proceeds to 70040. In accordance with the present principles, data from the Flowsheet to be displayed in or hidden from the medical records dashboard can be filtered using the identified Specialty Configurations.

At 70040, it is determined if at least one Custom Configuration exists. If so, the process proceeds to 70050 during which all Custom Configurations are identified such that the data from the Flowsheet is filtered to only display or hide, collapse or expand, gray out or alert by any means, data associated with the identified Custom Configurations. For example, in some embodiments custom configurations and data to be displayed in, hidden from, or alerted by any means, the medical records dashboard dependent on the custom configurations can be predetermined and stored in the Rules Engine 10180 of FIG. 62. Alternatively, or in addition, in some embodiments, a user can use a user interface associated with the medical records dashboard to create and/or identify custom configurations. In accordance with the present principles, Custom Configurations can require certain portions, columns, and/or rows of the medical records dashboard to be displayed or hidden. After the Custom Configurations are identified and/or if it is determined that a Custom Configuration does not exist, the process illustratively proceeds to 70060. In accordance with the present principles, data from the Flowsheet to be displayed in or hidden, collapsed or expanded, grayed out or alerted by any means, from the medical records dashboard can be filtered using the identified Custom Configurations.

At 70060, it is determined if at least one predefined Appointment Type exists. That is, in some embodiments, appointment types can be identified that, no matter what rules indicate that certain data should not be displayed or hidden, collapsed or expanded, grayed out or alerted by any means, the identified appointment types are to be displayed or hidden, collapsed or expanded, grayed out or alerted by any means, in at least one location of a medical records dashboard of the present principles. In some embodiments, appointment types can be identified and stored in, for example, a storage accessible by the Rules Engine 10180 of FIG. 62. Alternatively, or in addition, a user can identify appointment types using a user interface associated with a medical records dashboard of the present principles. If it is determined that at least one specified appointment type exists, the process proceeds to 70070 during which the appointment types are identified such that any data from the Flowsheet identified as a specified appointment type can be displayed or hidden, collapsed or expanded, grayed out or alerted by any means, in at least one portion of a medical records dashboard of the present principles. In accordance with the present principles, appointment types can require certain portions, columns, and/or rows of the medical records dashboard to be displayed or hidden, collapsed or expanded, grayed out or alerted by any means. After the appointment types are identified or if it is determined that a specified appointment type does not exist, the process illustratively proceeds to 70080.

At 70080, it is determined if at least one predefined Procedure exists. That is, in some embodiments, procedures can be identified that, no matter what rules indicate that certain data should not be displayed or hidden, collapsed or expanded, grayed out or alerted by any means, the identified procedures are to be displayed or hidden, collapsed or expanded, grayed out or alerted by any means, in at least one location of a medical records dashboard of the present principles. In some embodiments, procedures can be identified and stored in, for example, a storage accessible by the Rules Engine 10180 of FIG. 62. Alternatively, or in addition, a user can identify procedures using a user interface associated with a Data Command Center of the present principles. If it is determined that at least one specified procedure exists, the process proceeds to 70090 during which the procedures are identified such that any data from the Flowsheet identified as a specified procedure can be displayed or hidden, collapsed or expanded, grayed out or alerted by any means, in at least one portion of a medical records dashboard of the present principles. In accordance with the present principles, procedures can require certain portions, columns, and/or rows of the medical records dashboard to be displayed or hidden, collapsed or expanded, grayed out or alerted by any means. After the procedures are identified or if it is determined that a specified procedure does not exist, the process illustratively proceeds to 70100.

At 70100, it is determined if at least one predefined Specialty or Physician exists. That is, in some embodiments, specialties or physicians can be identified that, no matter what rules indicate that certain data should not be displayed or hidden, collapsed or expanded, grayed out or alerted by any means, the identified specialties or physicians are to be displayed or hidden, collapsed or expanded, grayed out or alerted by any means, in at least one location of the medical records dashboard. In some embodiments, specialties or physicians can be identified and stored in, for example, a storage accessible by the Rules Engine 10180 of FIG. 62. Alternatively, or in addition, a user can identify specialties or physicians using a user interface associated with a medical records dashboard of the present principles. If it is determined that at least one specified specialty or physician exists, the process proceeds to 70110 during which the specialties or physicians are identified such that any data from the Flowsheet identified as a specified specialty or physician can be displayed or hidden, collapsed or expanded, grayed out or alerted by any means. In accordance with the present principles, specialties or physicians can require certain portions, columns, and/or rows of the medical records dashboard to be displayed or hidden, collapsed or expanded, grayed out or alerted by any means. After the specialties and physicians are identified or if it is determined that a specified specialty or physician does not exist, the process illustratively proceeds to 70120.

At 70120, it is determined if at least one predefined Diagnostic Test exists. That is, in some embodiments, diagnostic tests can be identified that, no matter what rules indicate that certain data should not be displayed or hidden, collapsed or expanded, grayed out or alerted by any means, the identified diagnostic tests are to be displayed or hidden, collapsed or expanded, grayed out or alerted by any means, in at least one location of a medical records dashboard of the present principles. In some embodiments, diagnostic tests can be identified and stored in, for example, a storage accessible by the Rules Engine 10180 of FIG. 62. Alternatively, or in addition, a user can identify diagnostic tests using a user interface associated with a medical records dashboard of the present principles. If it is determined that at least one specified diagnostic test exists, the process proceeds to 70130 during which the diagnostic tests are identified such that any data from the Flowsheet identified as a specified diagnostic test can be displayed or hidden, collapsed or expanded, grayed out or alerted by any means, in at least one portion of a medical records dashboard of the present principles. In accordance with the present principles, diagnostic tests can require certain portions, columns, and/or rows of the medical records dashboard to be displayed or hidden, collapsed or expanded, grayed out or alerted by any means. After the diagnostic tests are identified or if it is determined that a specified diagnostic test does not exist, the process illustratively proceeds to 70140.

At 70140, it is determined if at least one Critical Condition exists. That is, in some embodiments, critical conditions can be identified that, no matter what rules indicate that certain data should not be displayed or hidden, the identified critical conditions are to be displayed in at least one location of a medical records dashboard of the present principles. In some embodiments, Critical Conditions can be identified and stored in the Rules Engine 10180 of FIG. 62. Alternatively, or in addition, a user can identify Critical Conditions using a user interface associated with the medical records dashboard 400. If it is determined that at least one Critical Condition exists, the process proceeds to 70150 during which the Critical Conditions are identified such that any data from the Flowsheet identified as a Critical Condition can be displayed. In accordance with the present principles, Critical Conditions can require certain portions, columns, and/or rows of the medical records dashboard to be displayed or hidden. After the Critical Conditions are identified or if it is determined that a Critical Condition does not exist, the process illustratively proceeds to 70160.

At 70160, it is determined if at least one Critical Procedure exists. That is, in some embodiments, critical procedures can be identified that, no matter what rules indicate that certain data should not be displayed or hidden, data associated with the identified critical procedures are to be displayed in at least one location of the medical records dashboard 400. In some embodiments, Critical Procedures can be identified and stored in the Rules Engine 10180 of FIG. 62. Alternatively, or in addition, a user can identify Critical Procedures using a user interface associated with the medical records dashboard 400. If it is determined that at least one Critical Procedure exists, the process proceeds to 70170 during which data associated the Critical Procedures are identified such that any data from the Flowsheet identified as being associated with a Critical Procedure can be displayed in at least one portion of the medical records dashboard 400. In accordance with the present principles, Critical Procedures can require certain portions, columns, and/or rows of the medical records dashboard to be displayed or hidden. After the Critical Procedures are identified or if it is determined that a Critical Procedure does not exist, the process illustratively proceeds to 70180.

At 70180, it is determined if at least one Risk Factor exists. That is, in some embodiments, Risk Factors can be identified that, no matter what rules indicate that certain data should not be displayed or hidden, the identified Risk Factors are to be displayed in at least one location of a medical records dashboard of the present principles. In accordance with the present principles, Risk Factors can require certain portions, columns, and/or rows of the medical records dashboard to be displayed or hidden. For example, a smoker with high blood pressure, and diabetes having an identified Risk Factor for a heart attack can require a visual field column with an alert to be displayed in at least a portion of the medical records dashboard 400. In some embodiments, Risk Factors can be identified and stored in, for example, a storage accessible by the Rules Engine 10180 of FIG. 62. Alternatively, or in addition, a user can identify Risk Factors using a user interface associated with the medical records dashboard. If it is determined that at least one Risk Factor exists, the process proceeds to 70190 during which the Risk Factors are identified such that any data from the Flowsheet identified as identifying a Risk Factor can be displayed in at least one portion of the medical records dashboard. After the Risk Factors are identified or if it is determined that a Risk Factor does not exist, the process illustratively proceeds to 70200.

At 70200, it is determined if at least one Key Diagnostic Result exists. That is, in some embodiments, Diagnostic Results that are considered Key can be identified that, no matter what rules indicate that certain data should not be displayed or should be hidden, data associated with the identified Key Diagnostic Results are to be displayed in at least one location of a medical records dashboard of the present principles. In accordance with the present principles, Key Diagnostic Results can require certain portions, columns, and/or rows of the medical records dashboard to be displayed or hidden. For example, if a lab returns a positive infectious disease test, data associated with that Key Diagnostic Result can be caused to be displayed in at least a portion of the medical records dashboard. In some embodiments, Key Diagnostic Results can be identified and stored in, for example, a storage accessible by the Rules Engine 10180 of FIG. 62. Alternatively, or in addition, a user can identify Key Diagnostic Results using a user interface associated with the medical records dashboard. If it is determined that at least one Key Diagnostic Results exists, the process proceeds to 70210 during which the Key Diagnostic Results are identified such that any data from the Flowsheet identified as being associated with a Key Diagnostic Results can be displayed in at least one portion of the medical records dashboard 400. After the Key Diagnostic Results are identified or if it is determined that a Key Diagnostic Results does not exist, the process illustratively proceeds to 70220.

At 70220 of the embodiment of FIG. 66, it is determined if at least one Future Order/Appointment exists. That is, in some embodiments, Future Orders/Appointments can be identified that, no matter what rules indicate that certain data should not be displayed or should be hidden, data associated with the identified Future Order/Appointment are to be displayed in at least one location of a medical records dashboard of the present principles. In accordance with the present principles, Future Orders/Appointments can require certain portions, columns, and/or rows of the medical records dashboard to be displayed or hidden. For example, if an Open-heart surgery is scheduled for the future, it can be desirable for all medical care providers to see the scheduled Open-heart surgery in at least a portion of the medical records dashboard regardless of a medical care provider's specialty. In some embodiments, Future Orders/Appointments can be identified and stored in, for example, a storage accessible by the Rules Engine 10180 of FIG. 62. Alternatively, or in addition, a user can identify Future Orders/Appointments using a user interface associated with the medical records dashboard. If it is determined that at least one Future Order/Appointment exists, the process proceeds to 70230 during which the Future Orders/Appointments are identified such that any data from the Flowsheet identified as being associated with a Future Order/Appointment can be displayed in at least one portion of the medical records dashboard. After the Future Orders/Appointments are identified or if it is determined that a Future Order/Appointment does not exist, the process illustratively proceeds to 70240.

At 70240, it is determined if Co-Management of at least one patient is allowed and if patient information sharing is allowed. That is, in some embodiments, Co-Management of patients can require certain portions, columns, and/or rows of the medical records dashboard to be shared or hidden amongst different users/medical care providers. For example, at 70250, if a medical records dashboard in accordance with the present principles is being used by multiple medical care providers to care for a patient, the patient's primary care physician is able to see lab results from a specialist if the specialist has shared at least the relevant portions of a medical records dashboard. In some embodiments, patient data/information to be shared and, as such, portions of a medical records dashboard to be shared can be identified and stored in, for example, a storage accessible by the Rules Engine 10180 of FIG. 62. Alternatively, or in addition, a user can identify patient data/information to be shared and, as such, portions of a medical records dashboard to be shared using a user interface associated with the medical records dashboard. If it is determined that Co-Management of at least one patient exists and if patient information sharing is allowed, the process proceeds to 70260 during which the existence of Co-Management of at least one patient and patient information sharing is identified such that any data from the Flowsheet identified as being associated with Co-Management and patient information sharing can be displayed in at least one portion of the medical records dashboard 400. After the Co-Management and patient information sharing is identified or if it is determined that Co-Management and patient information sharing does not exist, the process illustratively proceeds to 70270.

In the embodiment of FIG. 66, at 70270, it is determined if any of the collapsible portions, columns, and/or rows of the medical records dashboard contain no respective values (i.e., are empty). If it is determined that collapsible portions, columns, and/or rows of the medical records dashboard contain no respective values, the process proceeds to 70280 during which it is determined if there are any overriding rules to disallow collapsing or hiding portions, columns, and/or rows of the medical records dashboard. If it is determined that collapsible portions, columns, and/or rows of the medical records dashboard contain no respective values, and there are no overriding rules, the collapsible portions, columns, and/or rows of a medical records dashboard of the present principles containing no respective values proceed to 70290 and can be collapsed or hidden from display on a least a portion of the medical records dashboard. After all of the display configurations have been determined as described above, at 70300 the data of the Flowsheet to be displayed, as determined by the process of FIG. 66 described above, is displayed in the medical records dashboard. The process can then be exited.

In accordance with the present principles and as described above, in some embodiments, rules determine portions, columns, and/or rows of the medical records dashboard to expand or display based on predefined criteria, and also determine portions, columns, and/or rows of the medical records dashboard to collapse or hide based on the predefined criteria, and can also determine portions, columns, and/or rows of the medical records dashboard to flag or highlight based on the predefined criteria. For example, in some embodiments, the entirety of a patient's accessible records can be viewed. In some embodiments, the entirety of a patient's accessible records is evaluated against specialty and user-specific configuration criteria (e.g., Rules), actively collapsing or hiding portions, columns, and/or rows of the medical records dashboard deemed unnecessary for a user or specialty and actively enabling the display of portions, columns, and/or rows of the medical records dashboard deemed relevant to the user or specialty. In some embodiments, an intelligent Rules system actively determines which portions, columns, and/or rows of the medical records dashboard to display based on a user, a user's specialty, a patient, a patient conditions, a patient procedures, risk factors, diagnostic results, future orders, future appointments, values recorded, values not recorded, calculated values, and absolute values for display. In another embodiment, shared portions, columns, and/or rows of the medical records dashboard between medical care providers and facilities can be added or expanded based on preconfigured or point-of-sharing decisions made by the sharing medical care providers.

Although the embodiment of the process for intelligently expanding, collapsing, displaying, hiding, graying out, and/or alerting columns, rows and/or any other portion of the medical records dashboard of the present principles described with reference to FIG. 66 illustratively comprises specific Rules-based configurations, other embodiments of the process in accordance with the present principles can comprise any combination of some or all of the described Rules-based configurations and can also comprise other Rules-based configurations. Even further, those skilled in the art will appreciate that the order of operations denoted in the process above with reference to FIG. 66 can be non-linear and optimized based on usage and workflow. That is, order, inclusion, and omission can be intelligently determined based on accessibility of data, predefined configurations, real-time user selection, custom configurations, preferred practice patterns, and/or workflow.

In addition, although in the embodiment of the process for intelligently expanding, collapsing, displaying, hiding, graying out, and/or alerting columns, rows and/or any other portion of the medical records dashboard of the present principles described with reference to FIG. 66, the Rules are described as being stored in a storage accessible to the Rules Engine 10180 of FIG. 62, those skilled in the art will appreciate that rules and configurations of a process of the present principles can be stored in tables, accessed remotely via API or other digital communications technology, or generated on-the-fly as the result of calculations during the operations. Rules and configurations can be stored within the application or reference outside data sources. Rules and configurations can be altered by the user, in some embodiments, by the application, in some embodiments, and/or by outside resources.

In addition, although in the embodiment of the process for intelligently expanding, collapsing, displaying, hiding, graying out, or alerting columns, rows and/or any other portion of the medical records dashboard of the present principles described with reference to FIG. 66, it is described that upon rendering the Flowsheet, data populates within the columns specified, in some embodiments, further rules and configurations can apply post-rendering, based on data returned and/or calculated within columns. In addition, in some embodiments, manual manipulation allows for human interaction with the finally determined dataset. As such, a user can acknowledge and remove portions, columns, and/or rows of the medical records dashboard once they have been rendered. Removal of such portions, columns, and/or rows of the medical records dashboard can be one-time, or permanent unless a subsequent event retriggers the rendering of those portions, columns, and/or rows of the medical records dashboard, and such rendering can be patient-specific, provider-specific, location-specific, or otherwise tied to an event, condition, or trigger.

In one example of the process of the present principles, a dentist can access a Flowsheet for a patient with a rare blood disorder. As a dentist, the returned set of data to be displayed in accordance with a process of the present principles would ordinarily include data germane to dentistry, collapsing or hiding certain portions, columns, and/or rows of the medical records dashboard with no values present and/or deemed unnecessary. The dentist can have also chosen not to view certain portions, columns, and/or rows of the medical records dashboard as a matter of practice. In accordance with embodiments of the present principles, as a patient with a rare blood disorder, additional portions, columns, and/or rows of the medical records dashboard could be added to the display to reflect the patient's condition of the rare blood disorder and such information could be highlighted/flagged to alert a user as to the importance of the information being displayed.

In another example, an ophthalmologist sees a diabetic patient with no diagnostic testing for a chronic illness. As an ophthalmologist, the patient data ordinarily returned for display by a process of the present principles would ordinarily include data germane to ophthalmology, collapsing or hiding certain portions, columns, and/or rows of the medical records dashboard with no values present or data deemed unnecessary for display by the process. In some embodiments, the ophthalmologist can have also chosen not to view certain columns as a matter of practice. As a patient with a lapse in testing and underlying condition requiring testing, portions, columns, and/or rows of the medical records dashboard having no value present which would normally be collapsed/hidden, could now be expanded/displayed, and highlighted or flagged to draw the attention of a user to the lack of testing having been performed on the patient.

In a third example, a primary care physician (PCP) may wish to view an entire patient history. The patient history can consist of patient care provided by the PCP, patient care provided by doctors in the same office as the PCP, and patient care provided by specialists outside the practice that co-manage the patient and have shared data with the PCP. In this arrangement, the entire dataset is provided for viewing on the medical records dashboard for care provided by the PCP and doctors within the same practice, and a shared dataset can be provided for viewing on the medical records dashboard for care provided by the specialists. Columns with no values can be collapsed or hidden if no value exists as described above.

FIG. 66D depicts a flow diagram of a method for rules-based data display in a data command center comprising a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of dynamic data entry fields for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method beginning at 6602 during which patient data/information from the at least one patient database is received. The method can proceed to 6604.

At 6604, the received patient information is compared with configuration rules to determine which portions of the received patient data/information are to be displayed and which portions of the received patient data/information is not to be displayed in the medical records dashboard. The method can proceed to 6606.

At 6606, dynamic data entry fields of the medical records dashboard that are determined to not have any patient data to display are identified as collapsed data entry fields, unless another rule is determined to override said rule and uncollapses/expands them. Those determined to have patient data are displayed, unless another rule is determined to override said rule and collapses them. Those determined to be altered or augmented are altered or augmented, unless another rule is determined to override said rule, and as such, the overriding rule is applied. The method can proceed to 6608.

At 6608, Patient data/information is displayed in the data fields of the medical records dashboard in accordance with the configuration rules and data fields of the medical records dashboard identified as collapsed data fields are collapsed and not displayed. Data fields determined to be altered/augmented are altered/augmented. The method can then be exited.

In some embodiments the dynamic data entry fields identified as intelligently alerted by any means, grayed out, expanded, collapsed, displayed, and/or hidden columns, rows, fields, and/or any other portion of the medical records dashboard comprise at least one of a column, row, or panel of a medical records dashboard of the present principles.

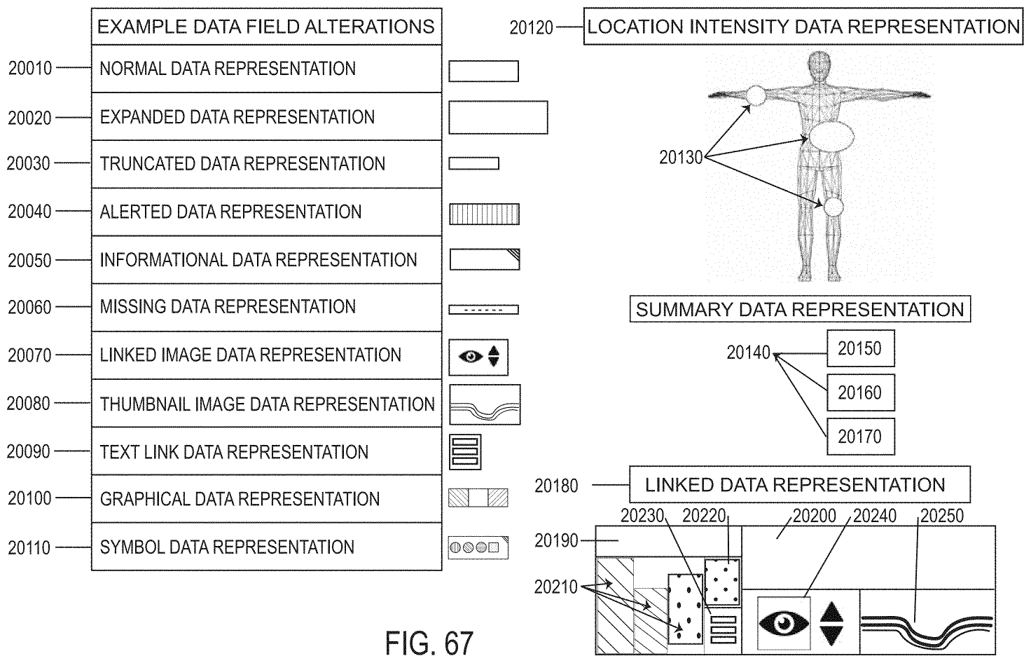

FIG. 67 depicts a graphical representation of examples of altered/augmented data representation in accordance with Dynamic Data Representation in accordance with an embodiment of the present principles. Dynamic Data Representation in accordance with the present principles can include but are not limited to:

Normal Data Representation 20010: As an example, a normal data field can simply exist as a box to contain data. It can also be represented as an icon, image, diagram, or any other means by which the data could be displayed without any alteration or augmentation.

Expanded Data Representation 20020: A data field can be expanded to show more data, or to draw attention to data contained within.

Truncated Data Representation 20030: A data field can be shrunken to hide less important data or to show the lack of data present.

Alerted Data Representation 20040: A data field can be highlighted by, for example, color, to draw attention to incorrect data, data exceeding a threshold, or critically important data.

Informational Data Representation 20050: A highlighted notification, such as the corner notification of FIG. 67, can be displayed and an associated note can identify more information as to why the notification is present.

Missing Data Representation 20060: A data field can have a series of dashes to indicate that although the data field was expected, the field is not filled to indicate missing data such as canceled or missed appointment data.

Linked Image Data Representation 20070: A data field can contain an icon and even a grading system to show that the field enables direct access to an underlying image, and can also indicate whether the image indicates whether there is an improvement or degradation from a prior image.

Thumbnail Image Data Representation 20080: A thumbnail of an image can be present within a data field offering a quick snapshot of, and direct access to, an underlying image.

Text Link Data Representation 20090: A data field can contain an icon to show direct access to text via the data field.

Graphical Data Representation 20100: A data field can represent underlying information as a graphical representation of the data.

Symbol or Icon Data Representation 20110: A data field can use a series of symbols or icons to represent underlying data in a way that the end user can interpret the symbols or icons to understand the underlying data.

Location Intensity Data Representation 20120: Data Representation is a general term to describe how to display an area which represents data. As such, representing the importance of key events in a graphical representation of a human body is also relevant. Differing representations can show location and intensity or importance of key areas being called out. In 20130, three different size and color data representations are depicted, which illustratively implement size and color to denote a relative intensity or importance of the data.

Summary Data Representation 20140-20170: Summary Data Representation illustrates a single data representation 20140 comprised of multiple data sources 20150-20160, with the ability to collate and summarize more than a single data source in a single representation. Summary data representations can offer total counts, highest and lowest values, best/worst values, and interaction within underlying data.

Linked Data Representation 20180: Linked Data Representation comprises multiple data representations in a combined representation. The example of Linked Data Representation 20180 as illustrated shows a Normal data representation 20190, next to an Expanded data representation 20200, above multiple Graphical data representations 20210, an Alerted data representation 20220, Linked Text data representation 20230, Linked Image data representation 20240, and a Thumbnail Image data representation 20250. Each individual data representation may affect the representation of any other, such that an alert 20220 can enlarge based on changes to the source data represented graphically 20210.

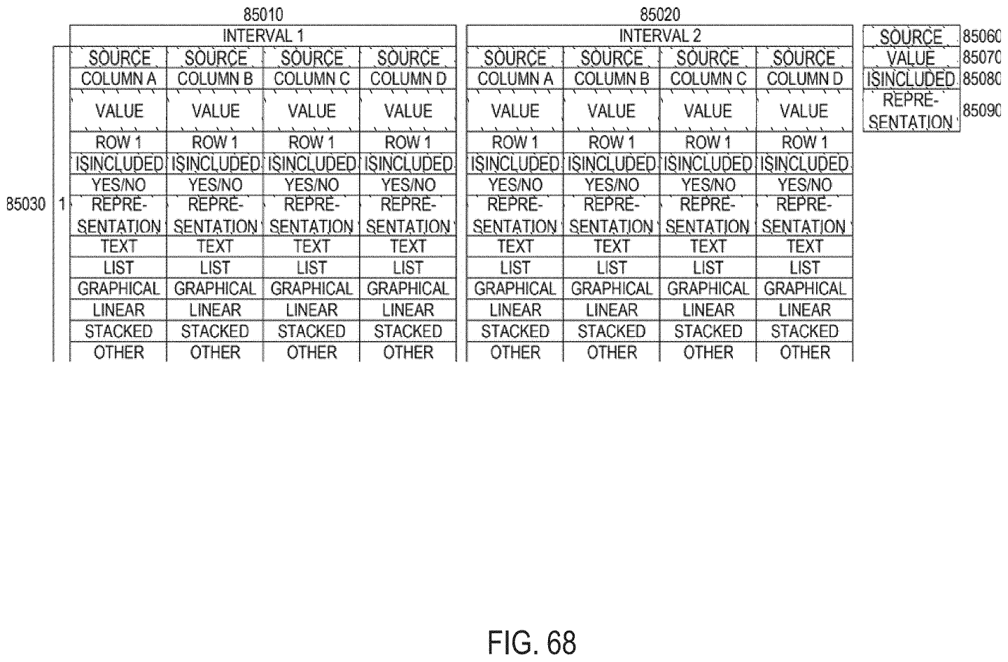

FIG. 68 depicts a Table depicting example configurations (data visualizations) of dynamic data fields in accordance with an embodiment of the present principles. In the embodiment of FIG. 68, data visualization is achieved with a series of configurations to determine what and how to display. For example, in one embodiment, Source Data (85060) consists of a Value (85070), an Inclusion/Exclusion Rule (85080), and a Visual Representation Configuration (85090). The data can consist of one type (85030), a series of data points collected, values captured, validated for inclusion, and visualized across 2 intervals (85010, 85020), correlated against additional types, a series of separate data points collected, values captured, validated for inclusion, and visualized across the same 2 intervals (85010, 85020).

FIGS. 69A and 69B depict a graphical representation of Dynamic Data Field Example Configurations. In some embodiments, in order to accomplish dynamic data representation, a series of configuration rules must be employed. An example of such rules are illustrated with respect to the embodiment of FIGS. 69A and 69B. In this example, configuration has been shown with three panels, General Configuration 32010, denoting high-level choices as to what will be displayed, Parameters 32060, denoting when to augment display of data, and Configuration 32110, describing how to augment display of data. It should be noted that in some embodiments such configuration can include more or less sections or options within sections, based on use case and requirements.

Under General Configuration 32010, Date Configuration 32020 is displayed with options for Chronological ordering from Oldest to Newest or Newest to Oldest. Time Period 32030 enables the configuration to show all available data, or just data within a defined start and end date. Location 32040 enables refinement of which data will be displayed for example, for a specific medical provider practice/specialty. Providers 32050 enables specification of individual or types of providers.

Parameters 32060 address reasons to show, hide, or augment data. Appointments 32070 enables user defined types of appointments to be configured. Details 32080 determines if more information should be displayed about the underlying data. Event Type 32090 enables augmentation to be defined based on important, or any, event. Editable 32100 determines if such data should be accessible for alteration, insertion, deletion, or other modifications.

Configuration 32110 addresses how to augment underlying data. Date Configuration 32120 enables for date format to be specified, when date is present. Display Duration 32130 can be used to define a time period within which data will display or the tying of the duration of display to an event or action. Size 32140 enables the ability to make data appear larger or smaller than standard display. Display Type 32150 enables for augmentation of underlying data by enabling the data to have visual and/or typographic alterations. Move 32160 enables data to appear in a different location. Direct Access 32170 enables data to link to other data, images, or access panels. Override 32180 enables configurations to override other, predefined configurations.

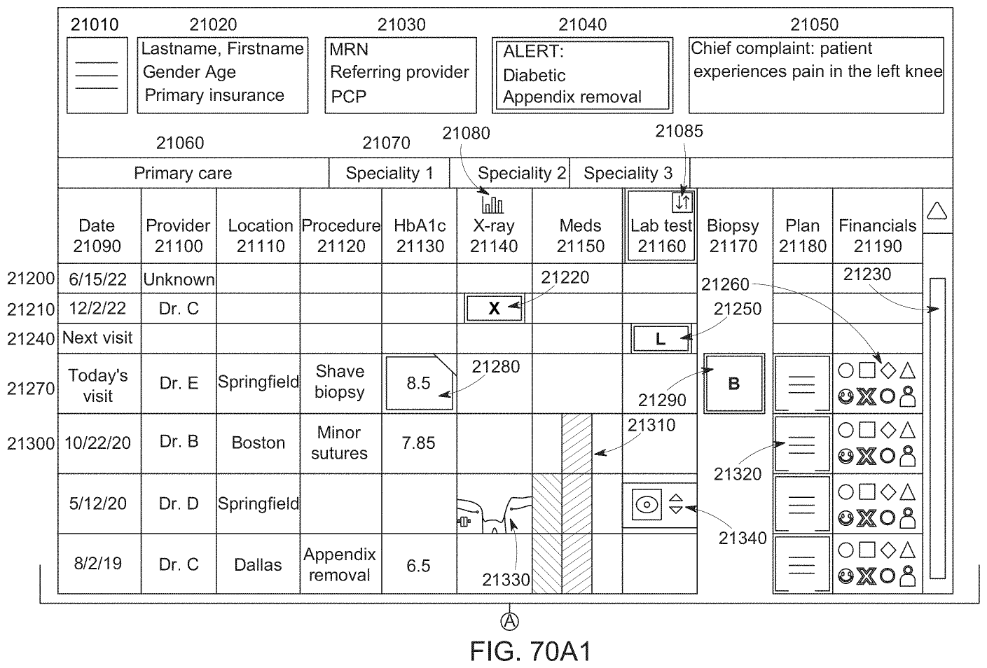

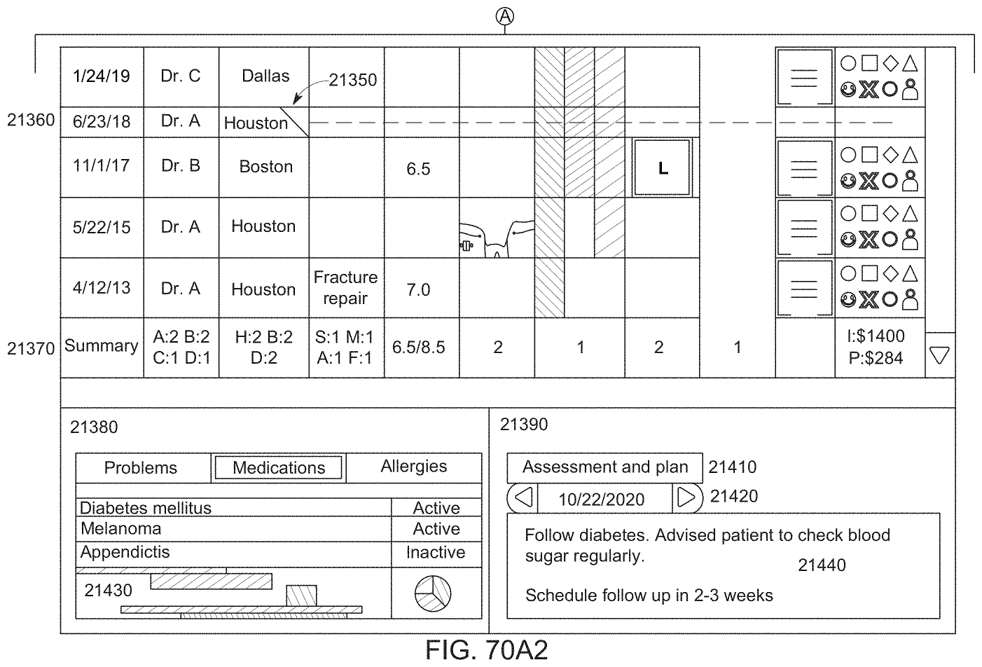

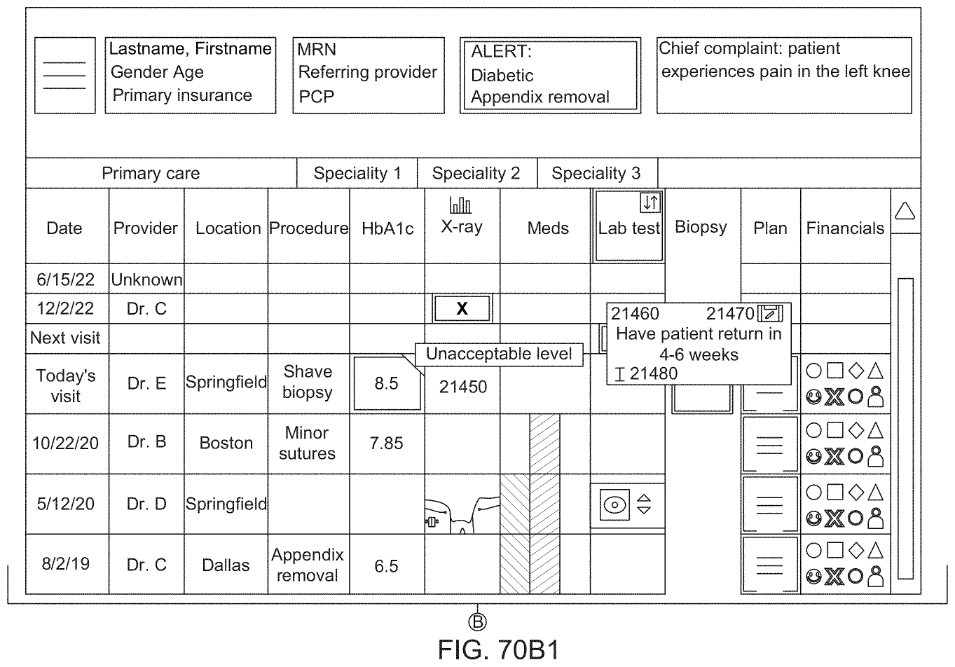

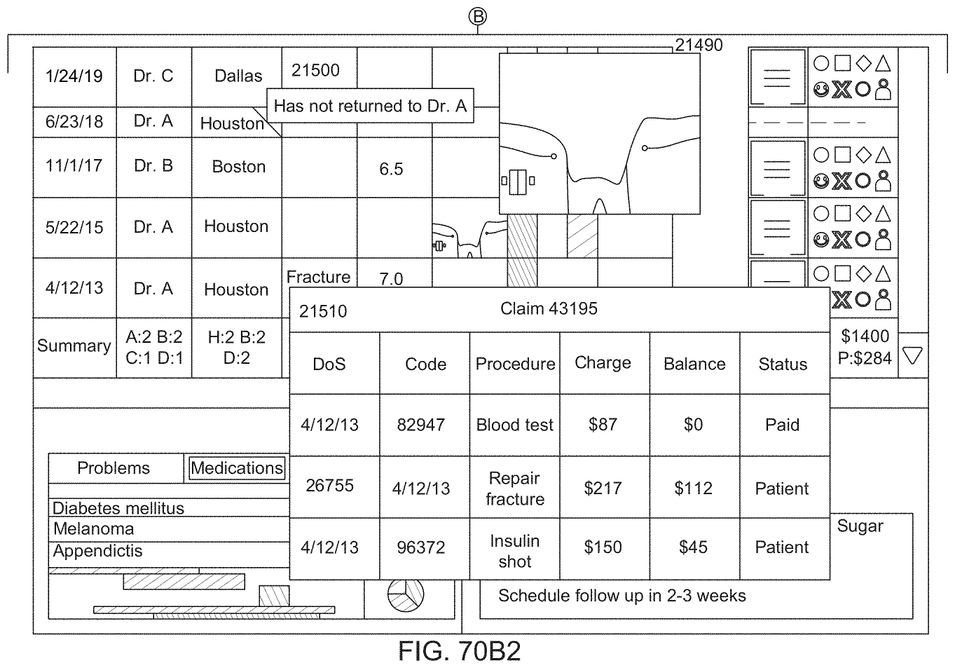

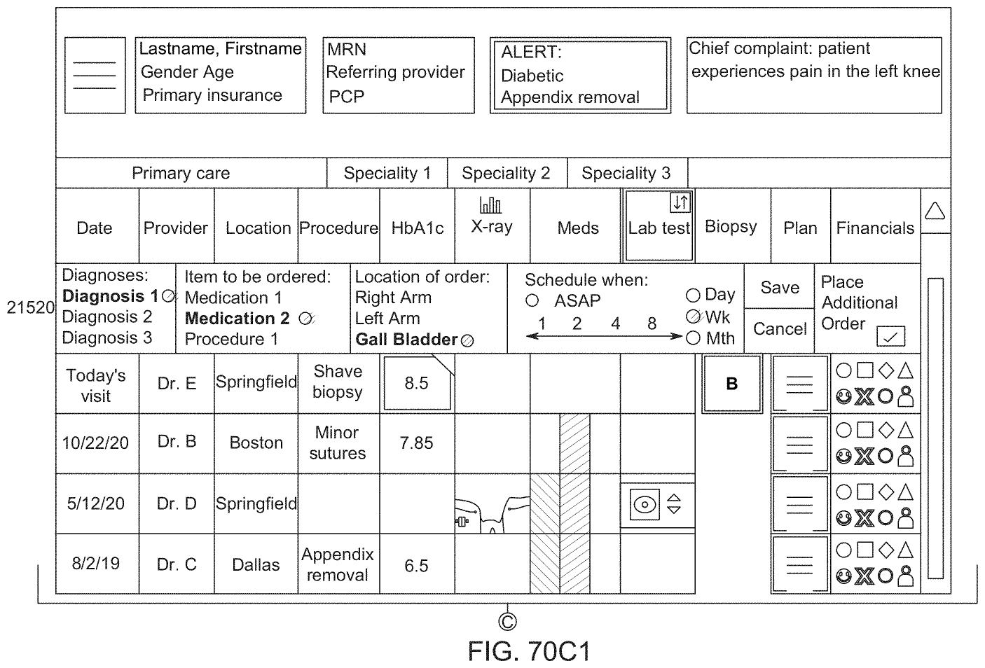

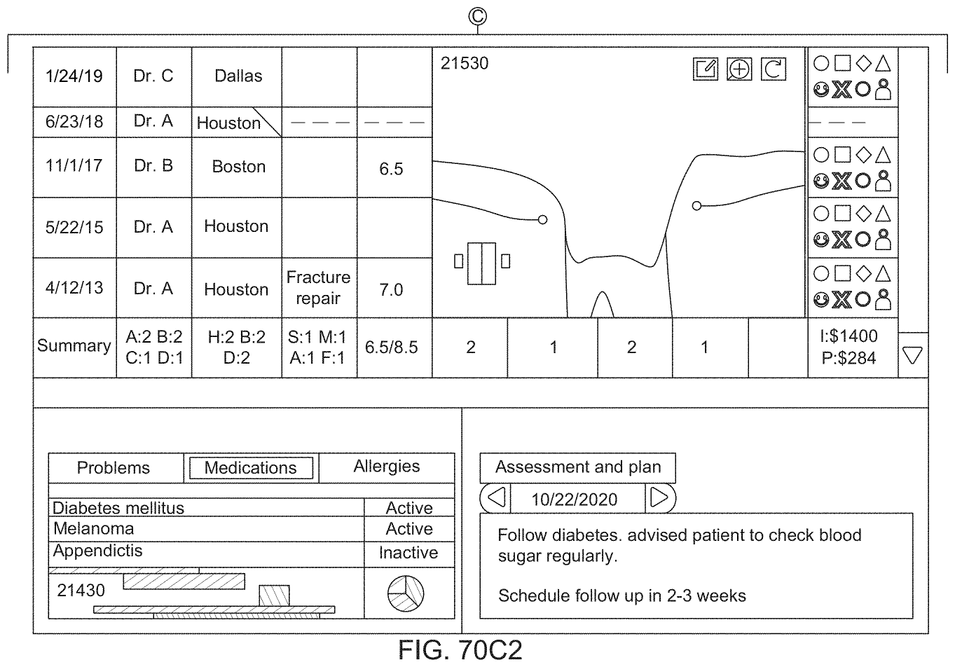

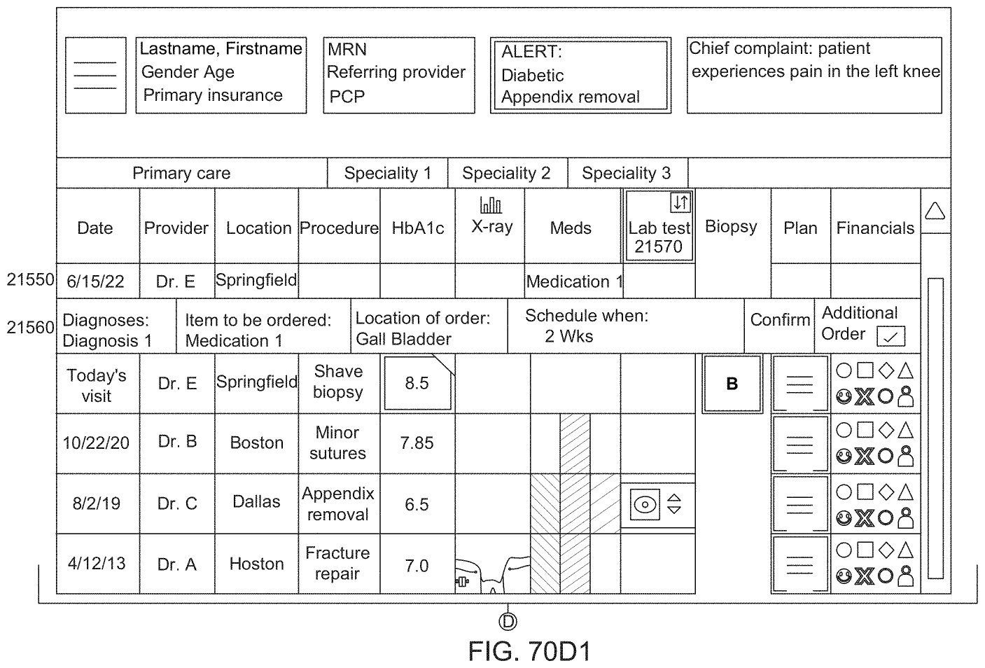

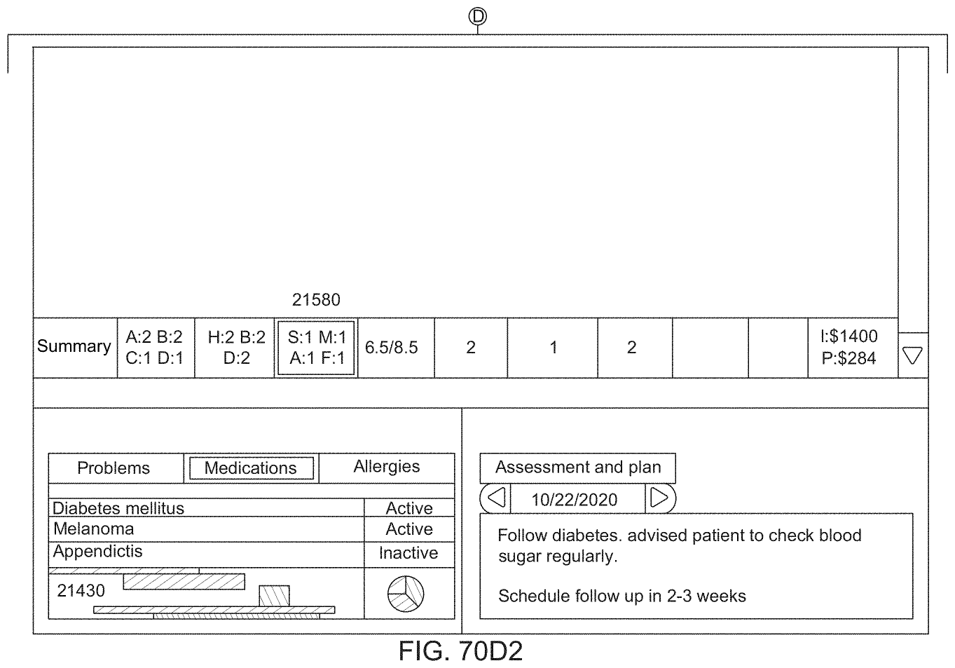

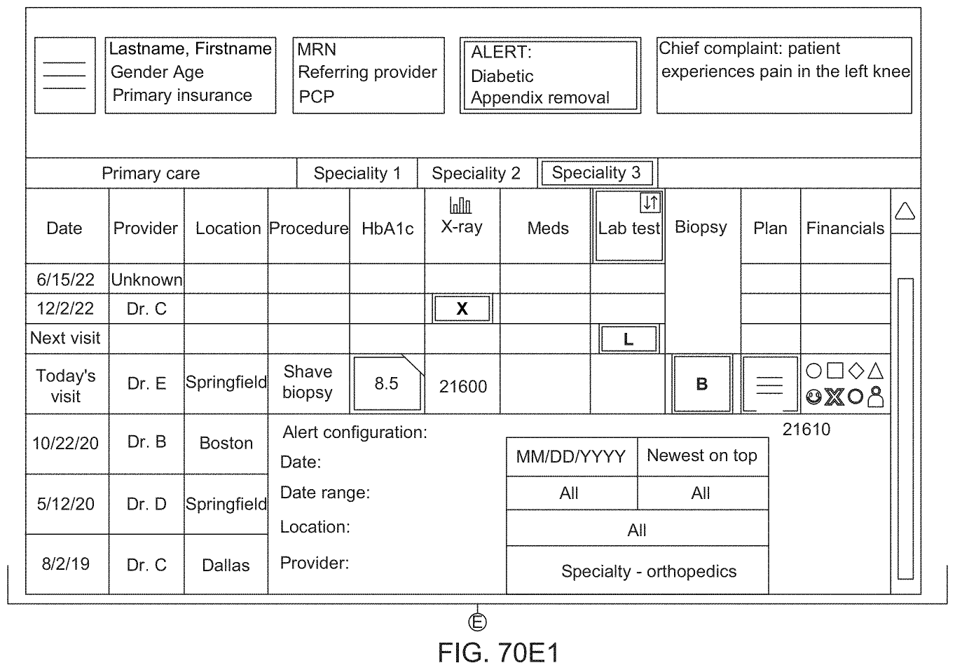

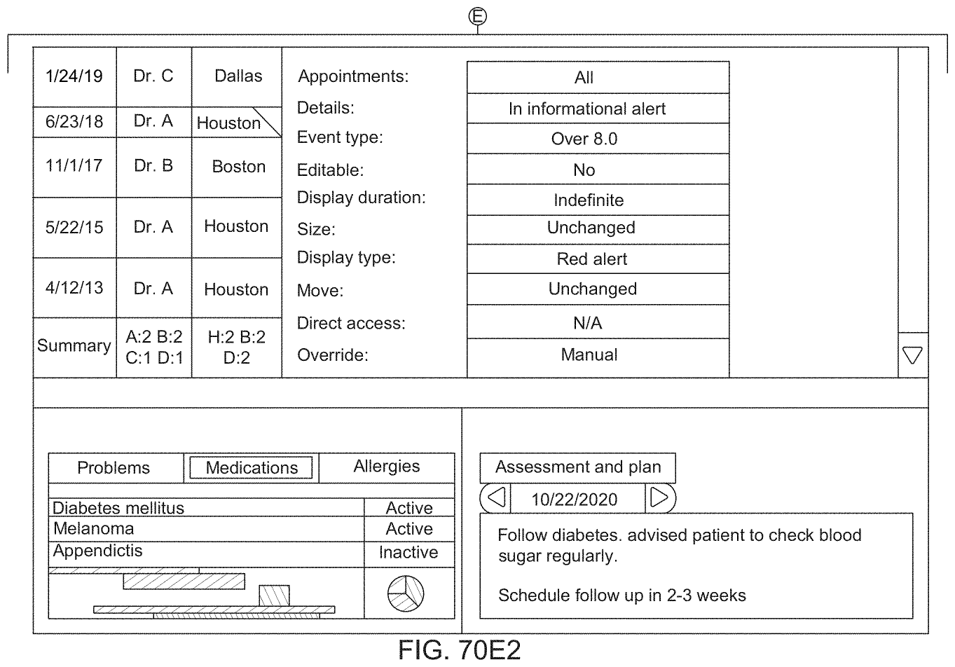

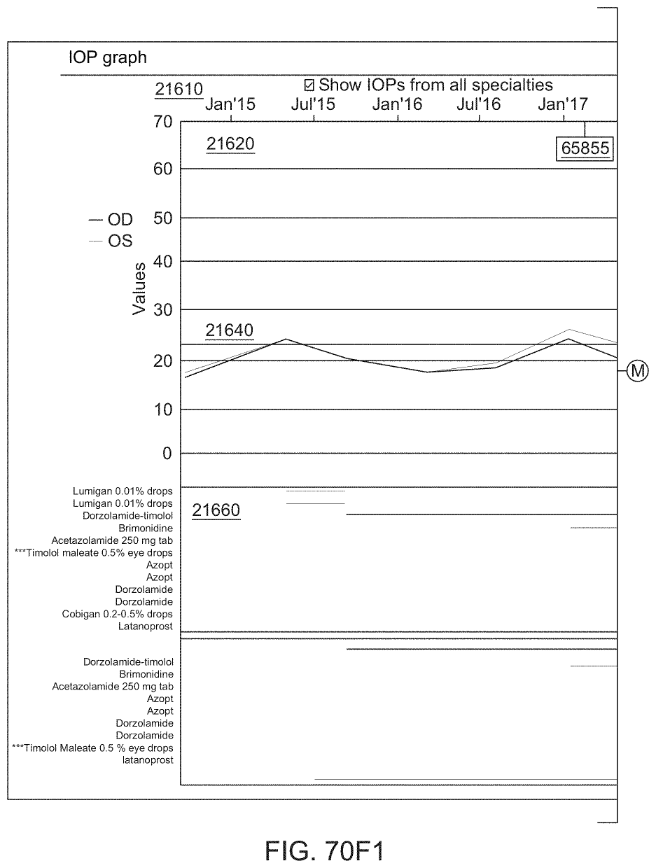

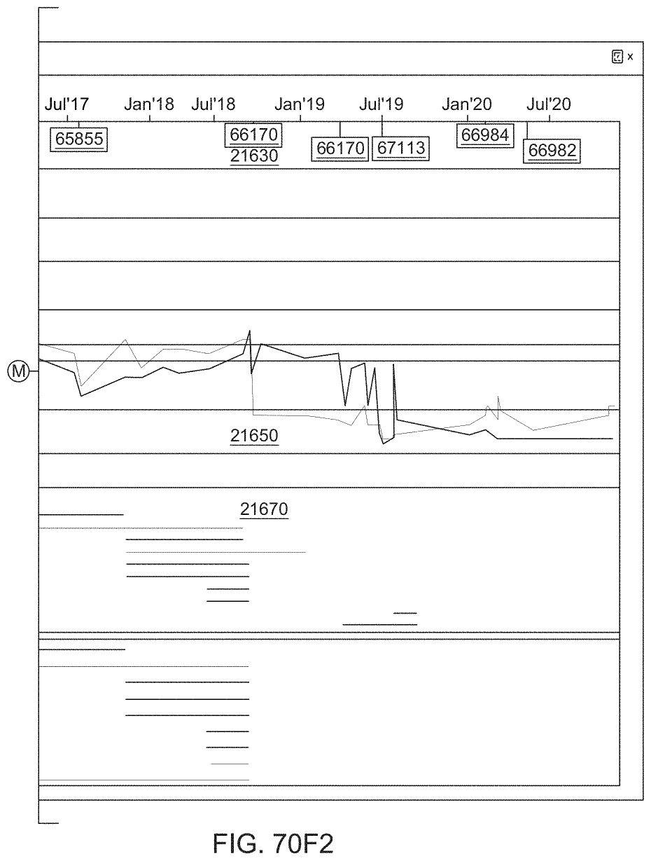

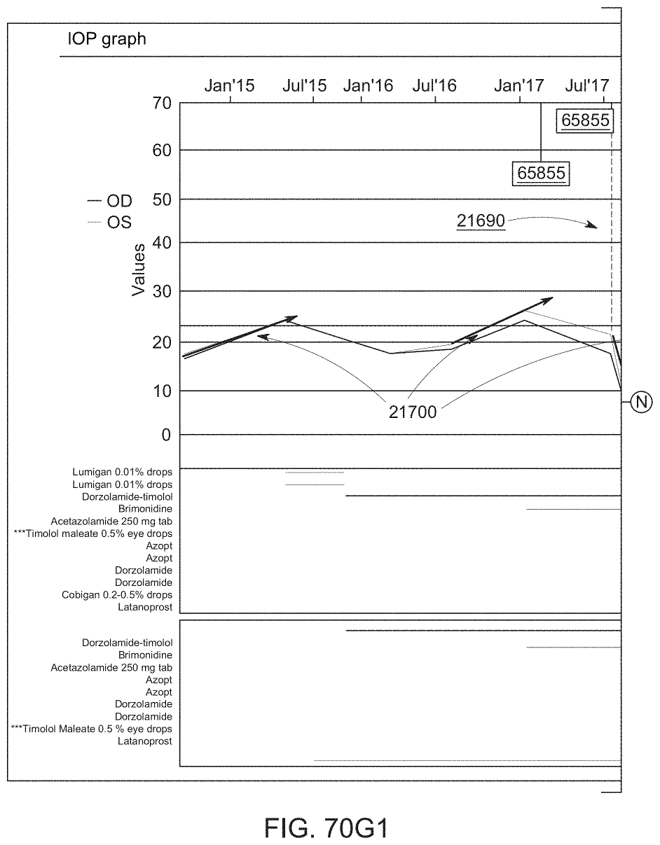

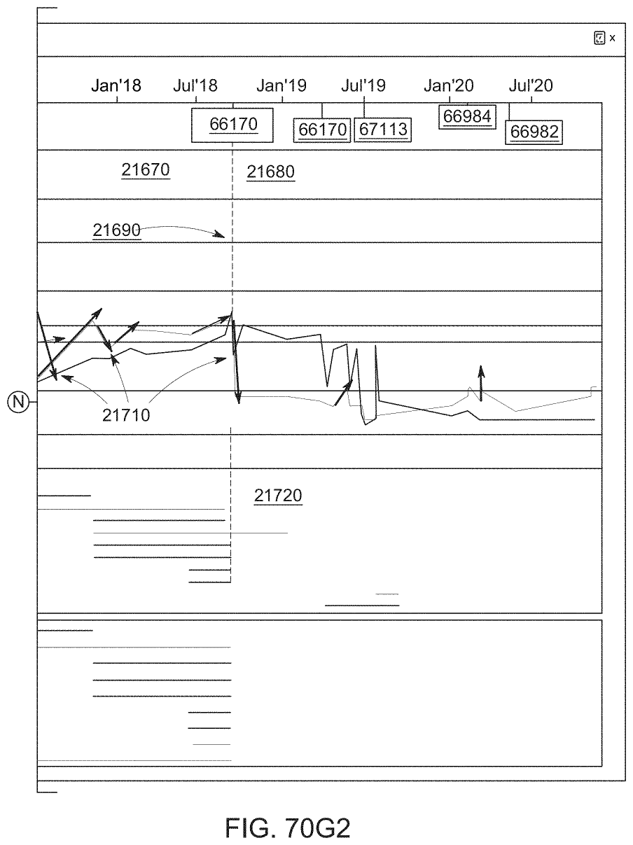

The representation of Dynamic Data of the present principles can take several forms, and exist within several different configurations, as is the nature of dynamic data representation. For example, FIGS. 70A1-70A2 depict a Patient-Specific Dashboard display of Dynamic Data in accordance with an embodiment of the present principles. In the embodiment of FIGS. 70A1-70A2, a Master Header Row is represented by 21060-21070, a Flowsheet Header Row is represented by 21090-21190, a Flowsheet is represented by 21200-21360, a Flowsheet Summary Row is represented by 21370, and two separate modules are represented by 21380 and 21390.

The Patient-Specific Dashboard of FIGS. 70A1-70A2 can be accessed by direct login to an application, launched from a separate application, either within the context of an existing patient from the application or generally through a patient search, or from within other aspects of a Command Center. A Master Header, in this example, is used to represent general data and information about a patient. A Navigation icon 21010 can be utilized to exit the Patient-Specific Dashboard, navigate to other aspect of a Command Center, or return to an initiating application. Patient Demographic and Insurance information can be represented in 21020. Medical Record Number and Associated Providers can be represented in 21030. Critical Patient Alerts can be represented in 21040, highlighted, such as in color, to draw attention to them. The Chief Complaint, or purpose for a visit, can be represented in 21050. In the embodiment of FIGS. 70A1-70A2 the key purpose of a Master Header is to denote the context within which all subsequent data representations exist.

The Master Header Row 21060-21070 can be utilized to differentiate different personas. In the embodiment of FIGS. 70A1-70A2, Primary Care 21060 is the selected Persona, and as such, data is represented in accordance with requirements of and for the Primary Care Persona. Beyond this persona, distinctions can be made per user or per other configurations, but the general governing persona remains Primary Care. Other Personas, such as Specialty 1, Specialty 2, and Specialty 3, collectively 21070, can be accessed through their respective fields, and can be highlighted to draw attention to important aspects of patient information which may not be represented within the selected Persona's view, such as Specialty 3 being highlighted. Personas can include, but are not limited to, user- or specialty-defined personas, practice defined personas, or any logical or geographical representation, not limited to a single user or practice, and inclusive of outside practice patient information.

The Flowsheet Header Row 21090-21190 denotes, in this example, column headers. Said Flowsheet Header Row is not limited to columnar data, and may exist vertically, or along any axis, or exist outside the context of an array of data. The Flowsheet Header Row, in this example, is comprised of Summary Data Representations 20140 of FIG. 67, accounting for all data in the underlying column or logical grouping to be represented by Summary Data Representations.

In the embodiment of FIGS. 70A1-70A2, the Flowsheet Header Row begins with Date 21090 representing the date of occurrence of key events. Provider 21100 represents an associated provider for such an event. Location 21110 represents the geographical location of the event. Procedure 21120 represents the occurrence of a medical procedure. HbA1c 21130 represents results from a medical test. X-Ray 21140 represents a diagnostic test. Within the X-Ray data field, an icon 21080 exists by which a user can launch a different representation of the presented data. Meds 21150 represents patient medications. Lab Test 21160 represents a laboratory test. In this example, the Lab Test data field is highlighted, for example in color, to denote an alert based on key data within the column, or logical grouping of information. Within the Lab Test field, an icon 21085 exists by which a user is enabled to launch a different interface, in this example, an Order Interface. Biopsy 21170, highlighted, for example in gray, represents a Flowsheet Header Row from a different Persona, in this example, Specialty 3, also highlighted, as this is important information which may not be normally viewable under the Primary Care Persona. Plan 21180 represents a provider's Assessment and Plan of Treatment for a patient. Financials 21190 represents the financial circumstances regarding the patient, patient's insurance, and other relevant financial information.

In FIGS. 70A1-70A2, the Flowsheet 21200-21360 is an array of patient information graphically represented in accordance with the Date 21090 column. At 21200, a Future Appointment is depicted, scheduled for a date, but not yet with a specific Provider. Such future appointments inform a provider as to future plans for either the provider, or any persona, to see the patient again. At 21210, we see a Future Order. Said order is scheduled for a Date, with a Provider, for an X-Ray 21220. At 21240, a Next Visit is depicted. In this example, the Next Visit is not scheduled for a Date, Provider, or Location. What has been specified for the Next Visit is a Lab Test 21250, which can occur at any Next Visit. Each of these three events, Future Appointment, Future Order, and Next Visit are all exemplified as Truncated Data Representations 20030 of FIG. 67. The Future Order of an X-Ray 21220 and Next Visit Lab Test 21250 are Linked Image Data Representations 20070 of FIG. 67, yet without the relevant data to link to, as they have not yet occurred. At 21230, a Scroll Bar is presented, denoting that there may be more information below the viewable portion, and provides access to the information.

Today's Visit 21270 is specific to an event occurring on the present Date and lists a Provider and Location. Also denoted for Today's Visit is a Procedure, a Shave Biopsy, which has occurred with Specialty 3 and has been added to the Flowsheet at 21290. An HbA1c result 21280 is an Alerted Data Representation 20040 of FIG. 67, as well as an Informational Data Representation 20050 of FIG. 67. As such, the field is Alerted by highlighting in red, and offers a colored (e.g. green) corner notification which can be interacted with to see more information. The Financial information 21260 is a Symbol Data Representation 20110 of FIG. 67. In this example, symbols may represent payment status of different categories, such as Office Visits, Procedures, and Diagnostic Tests, General Financial Status for the patient, Percentage of Deductible Paid, Likelihood of Insurance Denial, and Patient Responsible Balance. Symbols are configurable, and as such can be used to represent any data which may be relevant to a logical grouping of data represented.

Past Visits 21300 are shown below Today's Visit 21270. In this Past Visit, a Medication 21310 is denoted by a Graphical Data Representation 20100 of FIG. 67. In the embodiment of FIGS. 70A1-70A2 a colored bar (e.g., magenta bar) exists denoting the start and stop dates of the medication, between a first color represented (e.g. teal) medication and a second color represented (e.g., yellow) medication. Graphical representations of medications can conform to existing color specifications or custom configurations. The Plan 21320 is denoted by a Text Link Data Representation 20090 of FIG. 67. As such, interacting with the data will provide more information, such as the ability to read the full Plan.

At 21330, a Thumbnail Image Representation 20080 of FIG. 67 is depicted. A smaller view of an image is displayed to convey a quick overview of the underlying image. 21340 is a Linked Image Data Representation 20070 of FIG. 67. This provides enough information to inform the user that there is an underlying image, which can be directly accessed through interaction with the data representation.

At 21360, a Canceled Visit is depicted. Such a visit is shown as a Missing Data Representation 20060 of FIG. 67, to depict that there was an expectation of data, but it was not present. There was enough information to show a date, provider, and location, but because the patient did not arrive, nothing further is available. An Informational Data Representation 20050 of FIG. 67 shows in the Location logical grouping, which can convey whether the appointment was canceled by the practice or patient, missed, or rescheduled.

A Flowsheet Summary Row 21370 at the bottom of the Flowsheet is comprised of Summary Data Representations 20140 of FIG. 67, and can contain values such as how many visits occurred with each provider, at each location, how many of each type of procedure occurred, highest/lowest HbA1c results, total count of diagnostic tests, a summary of insurance patient balances, and the like. As a Summary Data Representation, each representation can be implemented to interact with data representations within the logical grouping of data associated with the field, such as filtering or placing an order.

A separate module 21380 exists in the bottom right, in the embodiment of FIGS. 70A1-70A2 to display Problems, Medications, and Allergies, 21400, a common subset of medical data. Problems is the selected tab, yet Medications is highlighted, for example in color, and as such, a representation of medications 21430 exists within the Problems tab, utilizing Linked Data Representation 20180 of FIG. 67. Such a module is a Linked Data Representation, as is the Module at 21390. Assessment and Plan 21410 denotes a main function of the module of FIGS. 70A1-70A2, yet it can be interacted with to change the function of the module, or rules can automatically determine the most important information to be represented. A Date Selector 21420 enables the context of this module to change to a selected date or can represent a date the user interacts with on the flowsheet. This exact information from the Assessment and Plan for the selected date is shown at 21440.

Several Dynamic Data Representations enable access to further information. For example, FIGS. 70B1-70B2 depict a Dashboard display of Dynamic Data enabling access to additional data in accordance with an embodiment of the present principles. The HbA1C Informational Data Representation at 21450 can be shown upon interaction using a hover, select, or other operation to select the relevant data. At 21450 of FIGS. 70B1-70B2, it is depicted that the HbA1c is at an unacceptable level. The Plan Text Link Data Representation interacted with at 21460 displays the full Assessment and Plan. In FIGS. 70B1-70B2, an icon at 21470 denotes the field is editable. As such, a text cursor is displayed at 21480 and the user can edit the plan. An Image Thumbnail Representation interacted with can display the full-sized image as seen at 21490. The Missed Appointment Informational Data Representation interacted with denotes the patient has not returned to see the provider their last appointment was scheduled with at 21500. The Financial information Symbol Data Representation interacted with shows a patient's charge status for the respective visit date at 21510. Each representation provides more information, as well as direct access and the ability to edit information.

FIGS. 70C1-70C2 depict an embodiment of a Dynamic Data Flowsheet in which the actual Flowsheet is being altered to allow for additional and/or different sized representations. At 21520, a full ordering panel is depicted as will be described in greater detail below. In the embodiment of FIGS. 70C1-70C2, however, future visits have been removed to make room for the ordering panel. Also represented within the ordering panel are colored alerts for Procedure, Medication, and Gall Bladder, to inform the user if there is a discrepancy in their selections. At 21540, a Thumbnail Image Representation is depicted expanded to take up several adjoining data representations to show the full image within the data representations.

FIGS. 70D1-70D2 depict an embodiment of a Dynamic Data Flowsheet including an order in the process of being placed. In the embodiment of FIGS. 70D1-70D2 space is made for a new row to be added at 21550, showing that Medication 1 is set to be ordered in accordance with the specified parameters. A colored notification exists at 21570 to inform the provider that the patient has an issue related to a Lab Test, such as one being indicated based on prior conditions and risk factors. At 21560, the intended order details are depicted, no longer alerted because the provider has properly selected compatible Procedure, Medication, and Location. The provider can now review the order, ensure that they have selected what they intended, and can confirm the order using the Confirm button. Additional orders can subsequently be placed by leaving the Additional Order check box checked.

In the embodiment of FIG. FIGS. 70D1-70D2, below the order, several events have now been hidden. Interacting with Summary Data Representation 21580, relevant data can be filtered. A single interaction can only display those rows with relevant values. For demonstrative purposes, a context menu 21590 is displayed to illustrate complex filtering functionality. In this case, Value of Procedures is selected, and as such, only events containing Procedures are now displayed. Several Summary Data Representation filters can be implemented at the same time, and can be configured to work based on user selection, such as instantiating the Order Process, which can automatically hide or show portions of the application relevant to the process.

FIGS. 70E1-70E2 depict an embodiment of a Dynamic Data Flowsheet including an Alerted Informational Data representation at 21600. That is, configuration of Dynamic Data Representations can occur within the context of the underlying data so as to ensure proper configuration is being entered. In the embodiment of FIGS. 70E1-70E2, an Alerted Informational Data representation is depicted at 21600. In some embodiments, such a representation can be configured by interaction directly with the field. Instantiating the Alert Configuration module 21610, parameters can be set in accordance with FIG. 68. Specific selections within this example show that for an Event Type of Over 8.0, a colored alert will display, and Details will show in an Informational Data Representation.

FIGS. 70F1-70F2 depicts a diagram of a correlative graphical representation of patient data able to be displayed by a Data Command Center in accordance with an embodiment of the present principles. That is, a Data Command Center of the present principles can collate, evaluate, and correlate multiple data sets including different types of data to represent them in a correlative graph, such as illustrated in FIGS. 70F1-70F2. In this embodiment, the correlative graph 21610 is comprised of three sections. The first section 21620 represents procedures by a code such as seen at 21630. The second section 21640 represents pressure, exemplified as a line graph which tracks with the pressure. A large drop in pressure is illustrated at 21650. The third section 21660 represents medications as graphical lines corresponding to each medication. A clear stop of multiple medications is shown at 21670. In the embodiment of FIGS. 70F1-70F2, these three factors, procedures, pressure, and medications, are depicted as inherently tied together in treatment.

FIGS. 70G1-70G2 depict correlations made for the diagram of the correlative graphical representation of patient data of FIGS. 70F1-70F2 in accordance with an embodiment of the present principles. More specifically, in FIGS. 70F1-70F2, at 21670, a procedure is depicted, now larger and highlighted, which correlates to a drop in pressure. A second, larger procedure 21680, now highlighted, for example in color, correlates to an even larger drop in pressure. These correlations are illustrated by the lines at 21690. Arrows of increasing size and differing color now represent notable rises and falls in pressure. At 21700, the first 2 arrows comprise a first color (e.g., yellow) and denote a moderate rise in pressure, while the third arrow comprises a second color (e.g., orange) and is thicker, denoting a significant increase. At 21710, colored arrows denote drops in pressure. The third arrow is clearly thicker, denoting a significant drop in pressure. It becomes clear that the procedure at 21690 is the significant factor in this drop. Another correlation is made at this point. At 21720, a connection to the cessation of several medications is depicted. In the past, such information would have existed in separate areas and associations would need to be made outside of the present application. In accordance with the present principles, in the embodiment of FIGS. 70F1-70F2 and 70G everything is clearly delineated, correlated, and displayed in such a manner as to reduce the amount of information needed to make connections, and as such, informed treatment decisions.

FIG. 71 depicts a Flowsheet depicting Header and Summary Row functionality as it pertains to Dynamic Data Sets in accordance with an embodiment of the present principles. A key feature of the Data Command Center is the ability for Flowsheet Header and Summary data representations to be programmed, or intelligently adapted based on rules defined in the Rules Engine 10180 of, for example, FIG. 62, to display notifications and allow interaction with the data that lies between them as describe in Summary Data Representation 20140 of FIG. 67. In the embodiment of FIG. 71, the Flowsheet Header consists of a Date 90010, Doctor and Location 90020, Visual Acuity 90030, Intraocular Pressure 90040, Procedures 90050, Injections 90060, Medications 90070, and an array of Diagnostic Tests 90080. Each header can behave differently based on the data contained within the represented data set, data contained within other data sets, data displayed, and/or data not displayed.

For the purposes of the embodiment of FIG. 71, Date 90010 is the date of an appointment, encounter, occurrence, or event which pertains to the treatment and care of a patient. Doctor and Location 90020 is comprised of any doctor or caregiver, and the location at which they interacted with the patient. Also displayable within this data set may be diagnostic testing equipment, equipment which can exist outside of the practice such as remote monitoring systems or take-home testing equipment, and/or anything that may provide a result or measurement which pertains to patient healthcare.

In the embodiment of FIG. 71, Visual Acuity 90030 depicts a summary of multiple Visual Acuity test results. Within the VA header, there are OD and OS column headers to accurately display the Right Eye (OD) and Left Eye (OS), yet any column can be set to a single group, multiple sub groups, and the sub groups can also contain multiple sub groups. At 90035, an expander is available within the header which will expand to show several data sets with distinct Visual Acuity results based on a number of methods for testing. It should be noted that the expander can be implemented to depict, or in the inverse, to hide, a number of data sets. The Expander may be set to Expanded—True, and all data sets will show unless the icon is selected to collapse them. The Expander can also be set to Expanded—False, in which case, all specified data sets will be collapsed unless the icon is selected to expand them. As seen in this example, one data set, VA, is set to expanded, while several other related data sets are set to collapsed. In another embodiment, the Expander can expand to show all or collapse to hide all, or any combination of showing and hiding data sets as denoted in configuration.

In the embodiment of FIG. 71, VA 90030 also contains a summary field 90100 denoting the best and worst values within the data set. Best VA is denoted in a first color (e.g., green), while Worst VA is denoted in second color (e.g., red). It should be noted that a series of configurations can be set to determine color, size, position, and purpose of any summary row. Summary Row data fields are interactive. In the summary field 90100, selecting the Best or Worst value may collapse all other fields to show only the value selected, as demonstrated in FIGS. 70D1-70D2. Fields can be expanded to show when a user deselects the summary field value.

Intraocular Pressure 90040 contains two sub data sets, OD and OS, to represent the right and left eye. Within the parent group, results of IOP testing are displayed. At 90045, one will note an icon of a graph. The icon can initiate a correlative graph of several key factors, including but not limited to, Date, IOP, Medications, and Procedures. An icon, such as in 21080 of FIGS. 70A1-70A2 can exist anywhere within a header, and on any portion of the application, to launch a different representation of the data.

In the embodiment of FIG. 71, IOP 90040 contains a Summary Data Representation 90110 displaying the highest recorded value in the column. It should be appreciated that the field has been configured differently than the VA summary field, and a series of configurations can be set to determine color, size, position, and purpose of any summary data representation.

In FIG. 71, Procedures 90050 can display any or all medical procedures performed on a patient. Procedures has a summary field 90120 which denotes a count of each type of procedure which was performed on a patient. Injections 90060 is a segregated group of procedures. It should be noted that any portion of the Data Command Center of the present principles can be configured to display any record, group of records, and calculations based on records. At 90065, a Header denotes the number of days since the last occurrence of an event in that data set. As with Procedures 90050, Injections 90060 also has a summary field 90130 showing the total count for each type of injection procedure within that data set.

In the embodiment of FIG. 71, Medications 90700 display in a graphical data representation. A single bar exists in the example because the patient is only on one medication. All other medications are hidden in accordance with dynamic data representation describe herein. A medication, in this example, displays as a bar beneath said header, originating at the start date and concluding at the end date. The summary field, unlike in previous examples, will display as many events as the medication takes up from the start date through the end date. All events which do not contain an instance of the medication bar will be collapsed until such time as the user deselects the summary field.

In 90080, a data set containing Fundus Photos displays a header denoting the amount of time that has elapsed since the last Fundus Photo occurred. At 2 years 3 months and 1 day, an alert is triggered by a rule which states the diagnostic test should occur within a specified timeframe, highlighting the header field in a different color. A corner informational data representation in the header allows for the user to view the precise reason for the alert. As no Fundus Photos have been performed in over 2 years, and with the display only showing the current year, the importance of the summary field 90140 becomes apparent. The summary field denotes a total of 5 Fundus Photos have occurred within the time of patient care, and the column header denotes there has been over 2 years since the diagnostic was performed. In FIG. 71, Summary fields 90100 through 90150 act and interact differently with the displayed data. Each serves a purpose, and while the purpose can differ, it should be noted that singularly, or in conjunction, all summary fields can be used to alter the display of data, and return said display to its original state.

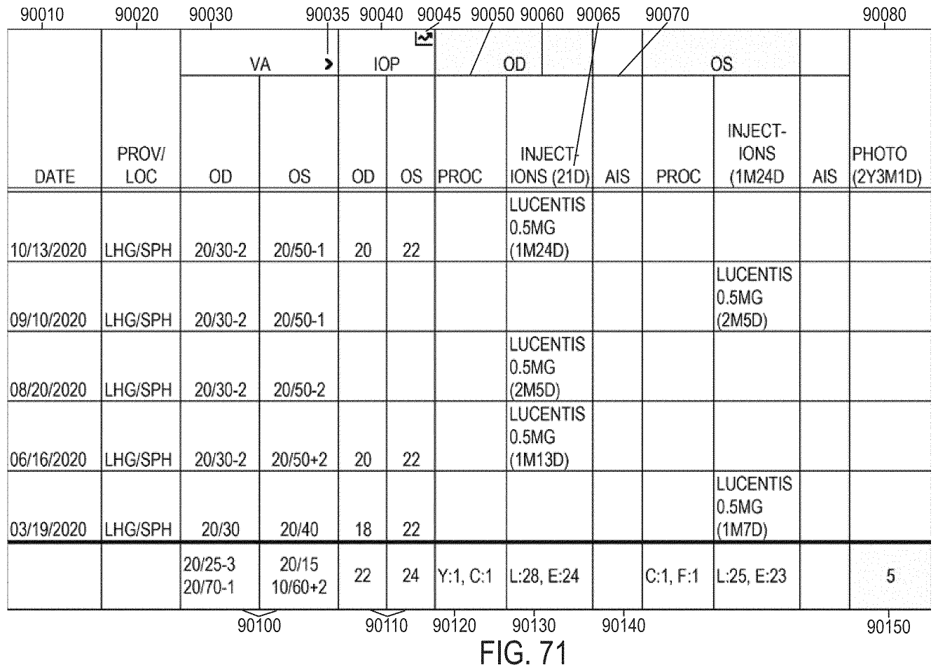

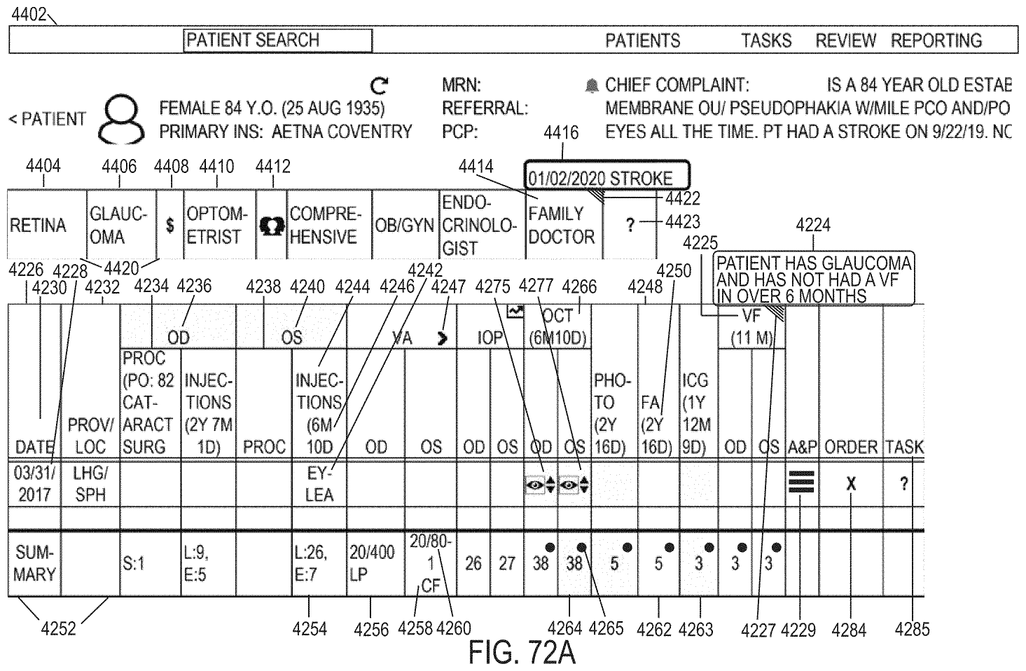

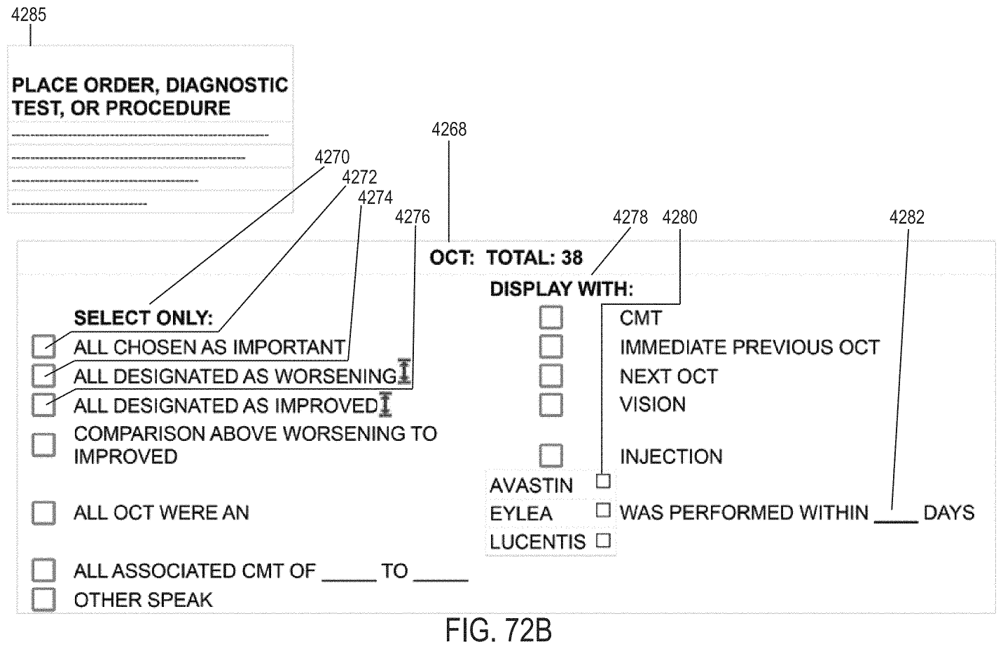

FIGS. 72A and 72B depict an alternate embodiment of a Flowsheet depicting Header and Summary Row functionality as it pertains to Dynamic Data Sets in accordance with an embodiment of the present principles. The embodiment of FIGS. 72A and 72B depict the functionality of three specific rows in the user interface in accordance with an embodiment of the present principles. That is, FIGS. 72A and 72B illustrates how three rows or panels in the user interface 4218 can convey a plethora of information for a healthcare professional in some embodiments. Header 4226 is an example of a header that can appear on each individualized specialty-based provider's actionable dashboard. As illustrated at 4220, different actionable dashboards that have been particularly designed for different providers of different specialties can be accessed. In the embodiment of FIGS. 72A and 72B, a summary row 4252 can be provided on each individualized dashboard for each doctor, specialist, or other user and can be specialized for each user. In the header 4226, the date is represented at 4230. The date can include a time, day, and/or date of a patient visit or the visit of a group of patients. 4232 can include the initials or name of the provider who cared for the patient or if just a location of testing, can include an indication of the location of the test performed. That is, in some embodiments, a medical care provider's initials can be presented at 232, which can also include a location, as providers can have multiple offices. In some embodiments, the information in 4232 can include an abbreviation, description, or identifying factor of which office a patient visited. In FIGS. 72A and 72B, 4228 shows an example of one of many patient encounters overtime.

In FIGS. 72A and 72B, 4236 depicts an example of a column that includes a procedure and, in the embodiment of FIGS. 72A and 72B, is divided into two different sides of a patient's body. In 4236 OD is displayed, which in eye care refers to a patient's right eye. In the case of orthopedics, 4236 could reference a patient's right knee. Similarly, an OS in 4240 represents the left column or in the case of eye care, the left eye, and in the case of orthopedics can refer to a left knee. As illustrated, the OS or left column of the header can be a procedure 4238. Under the column of the left eye is listed, by encounter, identifying procedures such as injections 4244. Item 4242 depicts an injection that has been performed (e.g., injection of Eylea is listed but can represent any data related to the column and row it is under), while 4228 shows the date of the medical encounter which by way of example shows Mar. 21, 2017.

As illustrated at 4234, the header 4226 is able to display that a cataract surgery has been performed and that a postoperative period is counting down. The header 4226 can also display that injections 4246 were last performed six months and ten days ago. 4224 can display a last time this test or item was performed on a patient or any kind of alert for instance if patient is allergic to the item or a condition impacts this test or procedure and 4224 can be on any column or row. Cell 4225 can have any highlights that can inform additional information such as an alert that something has not been done but should be in one year, such as orange, or if not done in two years that can be a severe warning so cell could be red. 4227 is a mechanism for user to learn more details and more information can come up for example 4227. In the embodiment of FIGS. 72A and 72B, 4224 depicts that a test was performed 2 years and 16 days ago. Header 4226 also includes a pop-up 4224 of underlying information. In the embodiment of FIGS. 72A and 72B, the patient has a diagnosis of glaucoma and has not had a visual field in over six months. 4247 shows an expanding mechanism where many more columns can suddenly appear. In this case, it appears in VA (vision) cell and there are many ways to test a vision, which can have different data. To save space on the display, the other methods are hidden until the user clicks on 4247 and then other columns are displayed. Clicking again on 4247 reverses the process and the columns are hidden again.

The summary row 4252 of FIGS. 72A and 72B depict how not only is the total number of something that had occurred in the rows above counted, but it can be divided according to what was performed. That is, in the embodiment of FIGS. 72A and 72B, summary row 4252 is a smart summary column. In the embodiment of FIGS. 72A and 72B, 4254 demonstrates that there were twenty-six Ls and seven Es of which one E (Eylea) is shown at 4242. The embodiment of FIGS. 72A and 72B depicts an example of a retina doctor who performs injections in the right eye in this case, and used “L,” which stands for Lucentis times and “E,” which stands for Eylea. 4256 similarly demonstrates the summary cell in a column of the left eye. 4260 shows the vision in the left eye is 20/80-1 and could also reflect the best number or event that occurred in the entire row overall of dates of service, and can be highlighted to inform the user that it is the best value. 4258 shows CF. In this illustration of a retina surgeon, CF means count fingers, which is very bad vision and is, therefore, red. For the first time in this illustration, a retina surgeon can know, over the time that the doctor has been delivering services, or any doctor, what is being reflected in encounters and rows above. The best vision was 20/80 (4260). The worst was count fingers (4258). This can also be the best blood pressure and the worst blood pressure. Every different specialty in medicine has different ways that it would like to measure the highs and lows in a column. 4262 simply shows the counting of the number that had occurred in all of the encounters. In this case five FAs 4248. Patients may be seen over a hundred times and many medical services are provided. 4252 can be implemented to first show the doctor summary and identify critical items for the doctor to consider and for the first time the user can instantly view whatever is critical. In some embodiments, the user simply clicks on any of the information in 4252 and instantly all the encounters related to that data clicked is displayed in the number of rows 4228 that are needed to show the data. If a user engages by any method 4262, five rows of 4248 (in this case FA), are displayed in 4228 with five rows of 4228, since there are five listed in 4262. If user also wants to view simultaneously 4263 (3 ICG listed), then three additional 4228 will be displayed. So, if 4262 and 4263 are clicked, a total of eight rows of 4228 will be revealed unless some of those two (2) tests were done at the same visit. Clicking again on 4262 and 4263 can reverse the process. 4202 provides another way for the user to sort information and display in 7126.

It is important to note that the Data Command Center of the present principles can measure anything in the row and display it in multiple different ways. The choice could be just to see the high and low as in 4260 and 4258 over a short period of one year or over as many years as there have been encounters. It can also be set to show percentage changes over time. In any case, this summary provides a tremendous amount of information to the provider for enabling rapid decisions.

A panel 4220 can be located at the top, side, or bottom of the display and can provide access for each specialist to different types of healthcare providers or different doctors who want to customize the display. Any type of doctor or dentist or other health care provider of any specialty can be listed. As few or as many as have actionable dashboards that can be accessed immediately with direct access by simply clicking on the specialist's name. For instance, the specialists can be retina 4220, glaucoma doctors 4206, or an optometrist 4210. All three happen to be types of eye doctors. All three could be in the same practice, separate practices, or even in different countries. Each, when clicked on, pulls up an actionable dashboard specially designed for them or their practice in that specialty. 4214 provides an example of a non-eye doctor, in this case, the family doctor.

It is important to note that any health care provider, if given permission by the patient, and each specialty noted in FIGS. 72A and 72B could see the actionable dashboard of the other specialists for as little or as much as each would allow. There is some information in an actionable dashboard to each specialist, practice, or doctor that they might not want others to see, which can be hidden (e.g., payments and costs). In addition, next to each actionable dashboard can also be additional information that can also be pulled up instead of the actionable dashboard itself. For instance, a dollar sign 4208 could be for providing for each practice or actionable dashboard, payments, costs, or any financial matter that can pop-up to show a different type of financial dashboard. 4212 shows an example that can pull up any type of additional information, such as a shared care dashboard between different providers.

4222 illustrates that an entire cell can alert all of the other providers of something important. It can be a color change, or flash, or blink. When activated, it represents that there is some type of important event, for instance, that all providers should know. A pop-up 4216 also can be shown at all times or by hovering over 4222. The popup could represent whatever the important item is to be alerted, for instance, a new diagnosis like that the patient had a stroke on 1/2/20, which all providers would like to know. It can also inform all providers that the patient missed an appointment that was particularly important with that doctor. So, that all specialists would know that and be able to remind the patient. The critical information in 4216 can also be inputted by creating a row 4228 in time order or as the first row that every provider views when they open their personalized flowsheet.

It will be further appreciated that the actionable dashboard can further include a communication center where users can send messages to each other in a HIPAA compliant way. A physician, while seeing a patient, can send a message in one of many ways for instance, by clicking 4223 and a mechanism to send a message to any other doctors caring for the patient, even if not in their own practice but another practice such as 4214 or 4210 and a message sent and added to that patient's actionable dashboard in the other practice. This mechanism can also set an alert, as seen in 4216 or allow any doctor when they believe something is so important for all providers to know to set a row in all providers tools and creating a row inserted. Doctors can also send messages within their own practice such as to their chief technician or the office manager to talk about following up on a patient or also to the billing office that there is a billing problem. Then, staff can report back to the doctor and this message can be imbedded into the smart actionable dashboard so that the next time a doctor sees the patient through icons and columns of correspondence of communication within the practice, the doctor can pull up what was the response to a message they had sent earlier. This response can be read live while treating the patient so that the doctor can take it into perspective while making decisions. The messaging system, attachments, or anything else can be sent to the doctor or health care provider in any way that they would want. Whether through email or the internal messaging system or as a tickler system within the EMR system that automatically toggles back and forth to the actionable dashboard, so the doctors can see their messages at the end of the day or the end of the week, or while seeing the patient. It really helps organize the doctor's life, so this actionable dashboard becomes the communication hub, the switchboard, for the entire practice, while communicating with the health care provider.

FIGS. 72A and 72B further depict an embodiment of how the interaction with a header and summary row can work. When a doctor interacts with the Flowsheet of the embodiment of FIGS. 72A and 72B and element number 4252 and clicks on the numbers. In another embodiment, colored dots can be depicted to enable a doctor to refine further exactly what they would want to see. Clicking on 4264 for instance on the summary row Element 4252 brings up just those encounters when that particular test was done and the 38 OCTs performed would come up and be displayed in 4228 where one such OCT encounter is depicted. If the user wants to sort further, then in one embodiment an example would be clicking on a colored dot in 4265 which enables a user to refine exactly what data the user needs to visualize to make decisions. For instance, upon activation a display panel can display on the screen when 4265 is activated. As such, a user can select 4270 and now many options can be selected to precisely sort and search what the user wants to find from the data. For instance, the user can choose all OCT's 4272 or just choose the OCTs that show worsening with the colored (e.g., red) arrows, 4274 which would select diagnostic tests with colored identification 4275 which could mean many things such as diagnostic tests with worsening result. The user can select to see only tests that are as designated as improved perhaps in this case as another colored (e.g., green) icon 4276. Only the OCTs that have that designation and the time of those encounters would be displayed. As can be seen, many different options on exactly how to refine which OCT can be selected.

In addition, other encounters can be selected to also be displayed along with the initial data encounters selected in 4270 of FIGS. 72A and 72B. Patients can have hundreds of visits and if a user wants to refine what they're looking for, the user can choose and pick out any particular diagnostic tests that share a common features as seen on FIGS. 72A and 72B, 4268, and the user can ask for other encounter dates and rows to be also sorted and displayed such as also showing if injections of Avastin, 4280, were performed within a certain time period 4282 of the OCT's, where criteria was selected to be displayed in 4270.

In FIGS. 72A and 72B, 4266 can be implemented to bring up an ordering panel 4285 or to expand other areas for ordering but always though one user interface and one display. Now the user is enabled to place an order while visualizing relevant data.

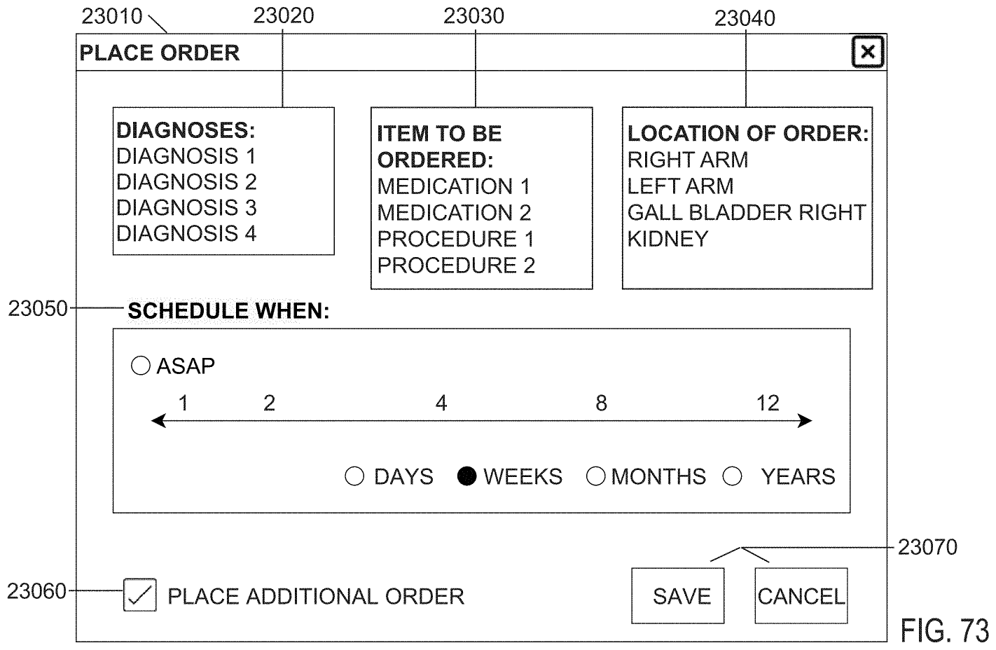

FIG. 73 depicts an example of an Ordering Panel of a Data Command Center of the present principles in accordance with an embodiment of the present principles. FIG. 73 illustrates the required elements for an Ordering Panel for the embodiment of FIG. 73. At 23010, an interface is available to place an order broken down into elements for placing a medical order in the context of Healthcare IT. Placing a medical order may include creating a medical order. Creating a medical order may also include reviewing or modifying a medical order. Diagnoses 23020 denote the condition or conditions of the patient requiring the ordered item. Codified in ICD-10 format or listed by common terminology, a healthcare professional can select one or more associated diagnoses for a patient. An item to be ordered 23030 can be a procedure, diagnostic test, medication, or other medical event for which an order is generally required. In addition to the event, a location can be required such as where on the body or which organ is to be the target of the order. This location can include specifications such as right or left side, which tooth, or other, more specific determinant than simply an arm or a leg. When placing an order, the date or duration can be specified 23050 to create an expectation of when the order is to be fulfilled. User Experience is also a factor in any technological solution, and as such, features can be included to enable easier use, access, or, in the case of 23060, the ability to place multiple orders without having to leave the order interface 23010. An order is completed, or placed, when a committal occurs, such as using the activation of the Save button, or ignored using the Cancel button 23070.

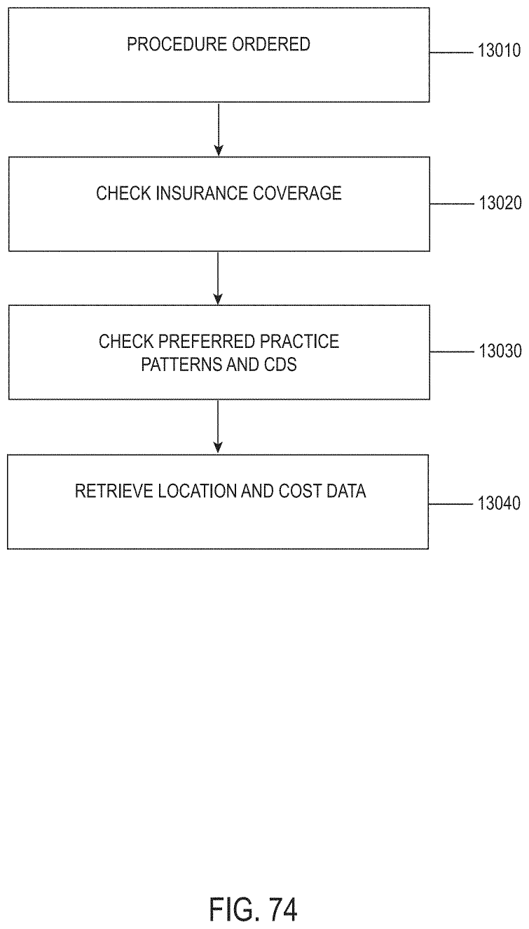

FIG. 74 depicts a flow diagram of an Order Processing method/algorithm in accordance with an embodiment of the present principles. For example, when a procedure is ordered at 13010, the Data Command Center of the present principles evaluates the insurance coverage at 13020. This process is described in greater detail below. Next, the Data Command Center evaluates the Preferred Practice Patterns and other clinical decision support rules at 13030. The last step in the process is to retrieve location, if applicable, and cost data 13040.

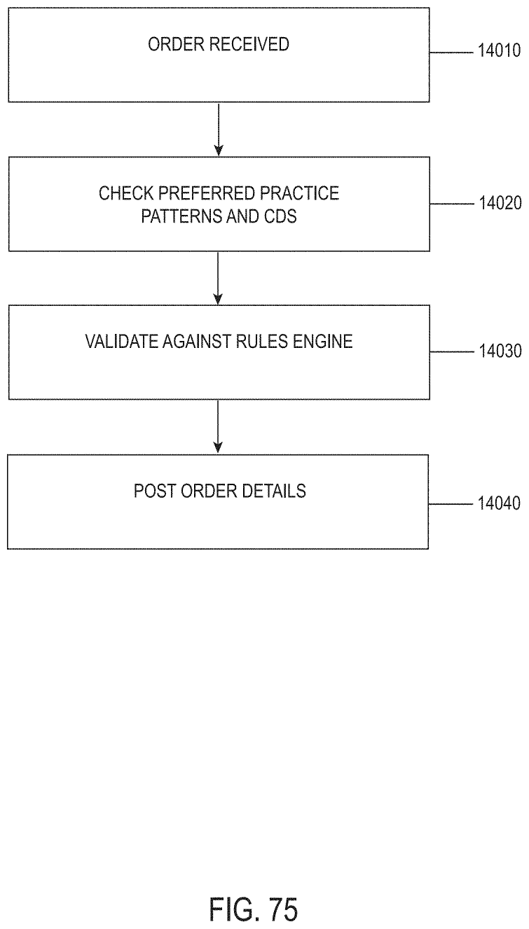

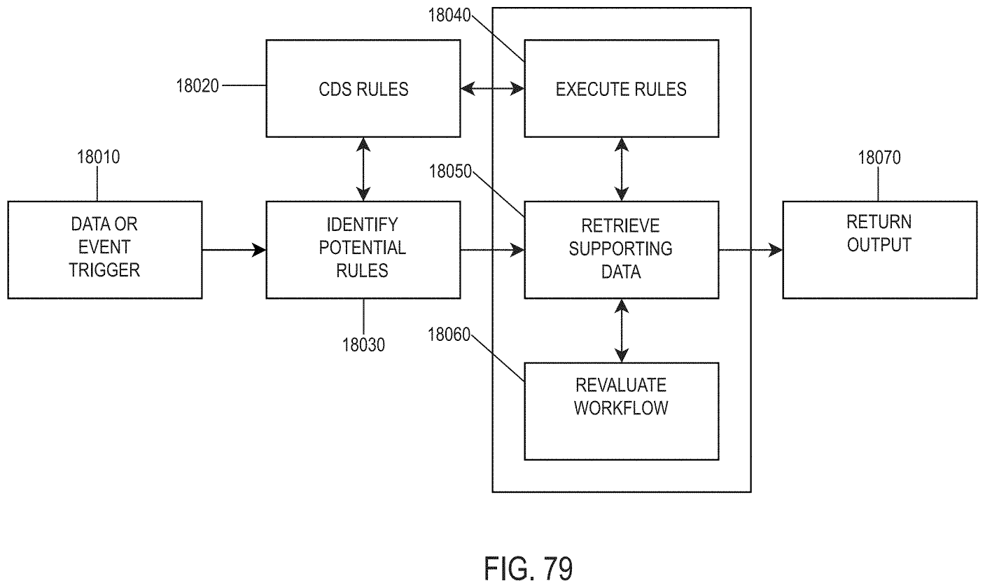

FIG. 75 depicts a flow diagram of an Order Receipt method/algorithm in accordance with an embodiment of the present principles. More specifically, FIG. 75 generally illustrates the process a Data Command Center of the present principles follows when order details are received from an external data source. When an order is received at 14010 the Data Command Center evaluates the Preferred Practice Patterns and other clinical decision support rules at 14020. Data gathered is then evaluated against the Rules Engine (for example 10180 of FIG. 62). Such processes are described in detail below with reference to FIG. 79. The last step in the process is to post and display relevant data to the user 14040.

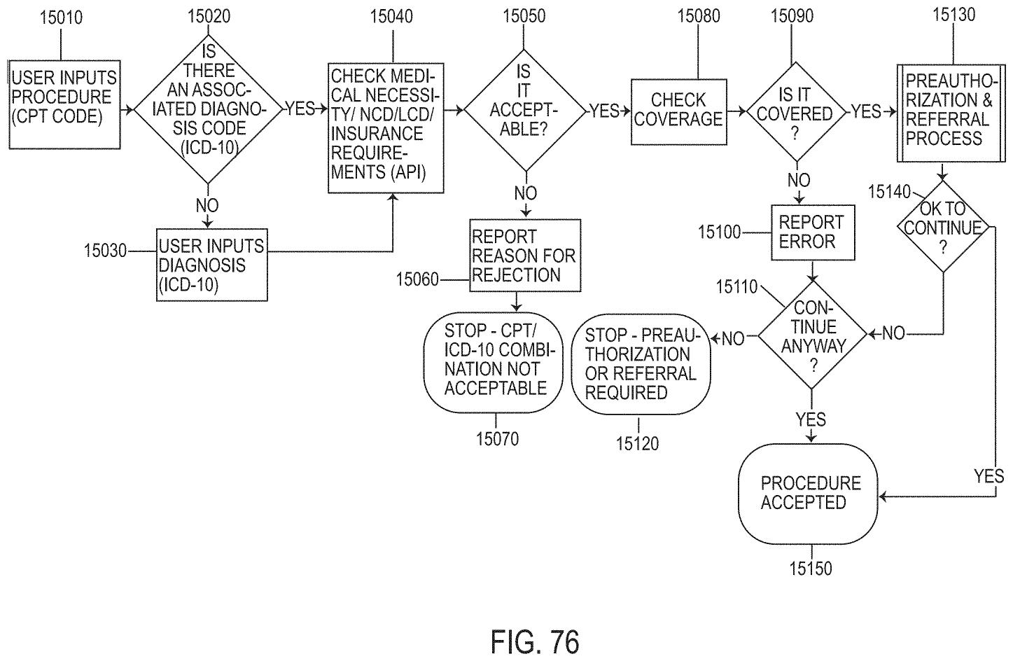

FIG. 76 depicts a functional block diagram of the logic used to check for pre-authorization and/or a referral of an Ordering Process. As illustrated in FIG. 76, when a new procedure is ordered (15010), the Data Command Center reviews the input data (15020) for an associated ICD10 Diagnosis code. If no ICD10 Diagnosis code is found, the user is prompted to provide one (15030). Once the ICD10 Diagnosis code has been associated with the CPT procedure code, the Data Command Center evaluates the data against the applicable guidelines (15040) using a third-party API and determines if the combination of CPT and ICD10 codes is acceptable (15050). If the input is not acceptable (15040), the user is notified of the disposition and provided with a rejection reason (15060). A rejection will terminate the process (15070). On the other hand, if the input is acceptable (15050), the Data Command Center will review the patient's coverage (15080) for the procedure using a third-party API. If the procedure is not covered by the patient's insurance (15090), the system will provide the user with an error code (15100). The patient will then have the option (15110) to continue with the procedure (15150) (knowing it will be paid for out of pocket) or to decline the procedure (15120), which terminates the process. If the procedure is covered (15090), the preauthorization and referral process is invoked (15130). If the preauthorization and referral process (15130) does not approve the procedure (15140), the patient will again have the option (15110) to accept the procedure (15150) (knowing it will be paid for out of pocket) or to decline the procedure (15120), which will terminate the process. If the preauthorization and referral process (15130) approves the procedure (15140), the patient can move forward with the procedure knowing it is covered (15150).

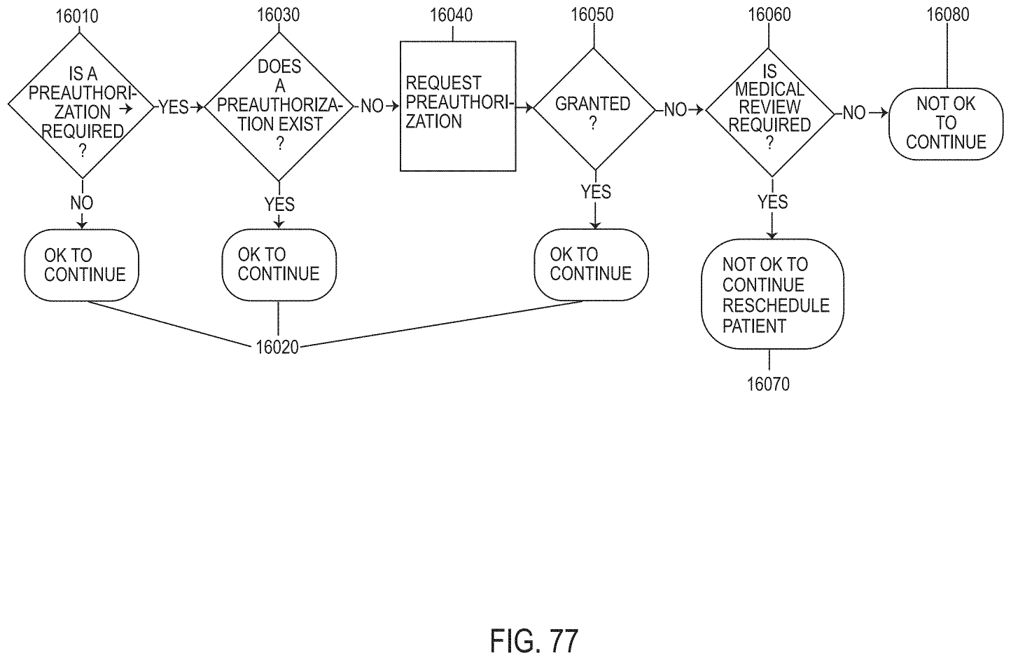

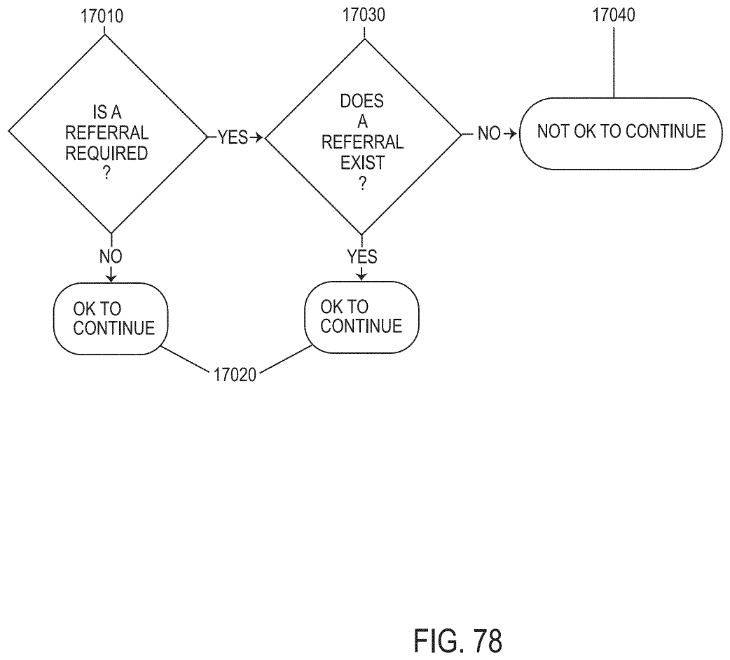

The preauthorization and referral process (15130) of the embodiment of FIG. 76 has two parallel processes, one which evaluates if the procedure requires preauthorization (FIG. 77) and another which evaluates if the procedure requires a referral (FIG. 78). The Precertification process described FIG. 77 is invoked by presentation of a CPT Procedure code and an ICD10 Diagnosis code. It is then determined using a third-party API whether or not a preauthorization is required (16010). If preauthorization (16010) is not required, then the process (16020) proceeds and the process returns an approval message (15140 from FIG. 76). If preauthorization 16010 is required, the system will then check for a pre-existing preauthorization (16030) using the third-party API. If a preauthorization exists, the authorization code is stored and the procedure can continue (16020) and the process again returns an approval message (15140 from FIG. 76). If a preauthorization does not exist, the system will request a preauthorization (16040), again using the third-party API. If the request for preauthorization (16040) is granted (16050), then the process (16020) proceeds and the process returns an approval message (15140 from FIG. 76). If the request for preauthorization (16040) is not granted (16050), then the Data Command Center will evaluate if the request for preauthorization (16040) needs to go through medical review (16060). If the request for preauthorization (16040) needs to go through medical review (16060), then the process (16070) does not proceed and the process escapes to the negative path of 15140 in FIG. 76 to determine whether to continue anyway. If the request for preauthorization (15140) does not need to go through medical review (16060), then the process (16080) does not continue and the process escapes to the negative path of 15140 in FIG. 76 to determine whether to continue anyway.

The referral process illustrated in FIG. 78 is invoked by presentation of a CPT Procedure code and an ICD10 Diagnosis code. It is then determined using a third-party API whether or not a referral is required (17010). If a referral (17010) is not required, then the process (17020) proceeds and the process returns an approval message (15140 from FIG. 76). If a referral (17010) is required, the system will determine if a referral exists. If a referral exists (17030), then the process (17020) proceeds and the process returns an approval message (15140 from FIG. 76). If a referral does not exist, then the process (17040) does not continue and the process escapes to the negative path of 15140 in FIG. 76 to determine whether to continue anyway.

The Command Center clinical decision support logic is implemented in a variety of methods. Pre-authorization, referral management, claims scrubbing, medical necessity checking for compliance with governmental and insurance regulations, and similar rules are embodied in the system through the use of third party systems. Internally, the Rules Engine (10180 of FIG. 62) is an implementation of the if-then style of clinical decision support.

More complex clinical decision support is illustrated in FIG. 79. In FIG. 79, the support is handled through the use of a RETE style (pattern matching, rules-based) algorithm. A RETE rules engine or inference engine embodies a set of rules built around a data model whereby when an event is triggered 18010 (data input or a new day starts) potential rules 18020 are identified (18030) that relate to the data/event. If rules are identified, they are executed (18040) along with the evaluation of workflow rules 18050 using supporting data retrieved (18060) as needed from the patient, insurance, clinical, financial, and imaging data repositories. Once complete, the outcome is returned (18070) to the requesting system for processing. Typically, the output is displayed on the screen for the user to consume or for storage in the designated database in the Command Center. In exemplary embodiments, the Command Center CDS illustrated in FIG. 79 is based on the Health Level (HL7) and Object Management Group (OMG) Decision Support Standards making it flexible and compatible with other similar systems. Those skilled in the art will appreciate that the inference engine may implement conventional artificial intelligence techniques such as those provided commercially by Watson Health and Truven Health Analytics, Inc. to process received data in connection with data repositories to provide diagnostic feedback and the like.

In some embodiments, a Data Command Center of the present principles enables the configuring of alerts in, for example, a medical records dashboard of the present principles. FIG. 47 depicts an embodiment of a medical records dashboard of a Data Command Center in which alerts and tasks can be performed in accordance with an embodiment of the present principles. Indicators such as diagnosis 20510, key results 20520, diagnostic tests 20530, and procedures 20540 are all key factors which can be used to trigger an alert or to send an automated task. In the diagnosis column 20510, glaucoma is indicated for the right eye (OD) and Ocular HTN is indicated for the left eye (OS) as required within the medical conditions dialog box 20550 as triggers. Thresholds are selected from a dropdown menu for key results 20560, for the results column 20520. Visual indicators can be selected 20570 to alert users of a threshold being met, exceeded, or falling below the specified level. A task panel 20590 can be utilized to set parameters at which an automated task will be sent and can involve triggers and time delays as to when the task will be sent 20600. Clinical tests can be validated and cross-referenced 20580 against a variety of conditions 20550, thresholds 20560, events 20610, and relevant factors 20620 to intelligently determine if an alert or task would be created.

FIG. 48 depicts a menu for pre-configuring alerts in accordance with an embodiment of the present principles. That is, in some embodiments, alerts can be configured ahead of time or at point of care for individual or groups of patients. That is, an example of configuring an alert is shown in FIG. 48. In the embodiment of FIG. 48, parameters are defined such as including specific diagnostic or CPT codes 20010, ignoring specific diagnostic or CPT codes 20020, required specific diagnosis or CPT codes 20030, parameters deemed necessary which can be necessarily true 20040 or false 20050, recommended diagnostic tests 20060, patient dependent situations 20070, defined by frequency in color (e.g., yellow) 20080, have a time interval within which to be performed in color (e.g., red) 20090, tasks can trigger if required diagnostic test is not completed 20100/20110, be aligned to a timeline 20120, have tasks auto-generated to a specific person or group 20130, and exceptions which prevent tasks from sending 20140.

In the example of FIG. 48, a Laser or Injection can be a trigger in the first column 20010. Glaucoma and Glaucoma Suspect would be required parameters in the third column 20030. The patient being on topical drops is a requirement deemed necessary in the first half of the fourth column 20040. Visual field, photo, and FAs are recommended tests in the fifth column 20060. Expiry and expiry based on seeing a retina specialist are examples of patient situation dependencies 20070. Frequency can be defined in color (e.g., yellow) for any time increment 20080. Required Timeline can also be specified in a color (e.g., red) for any time increment (20090). In the embodiment of FIG. 48, Tasks are specified to be created 20100 or not 20110 based on completion of the diagnostic test and Timeline can be specified in any time increment (20120). An individual or group can be selected as assignee 20130 and finally, the task trigger can be ignored based on specific criteria such as an IOP threshold or action taken 20140.

In accordance with the present principles, substantially any portion of a display, such as a medical records dashboard of the present principles, can be configured for an alert, including, but not limited to column headers and summaries, individual values within rows and/or columns, whole rows and/or columns, module headers, values displayed within modules, whole modules, and general application alerts outside of rows, columns, and/or modules. In some embodiments, alerts can be configured in accordance with Clinical Trials. Clinical trials exist throughout medicine often to determine efficacy of treatment and can include any number of other validations. As defined, alerts and auto-tasks can be configured for individual patients or groups of patients. In the specific case of clinical trials, a group of patients can be defined with specific criteria. As alerts can be configured by condition, procedure, risk factors, demographics, and a variety of other reasons, alerts can be used to isolate a group of patients deemed eligible for clinical trials. Often, clinical trials are based on medication efficacy.

FIG. 49 depicts an embodiment of a medical records dashboard in which alerts are configured based on medication in accordance with an embodiment of the present principles. As depicted in FIG. 49, alerts can be configured based on medication, instance count of medication, frequency of medication, and can also have secondary or tertiary responses to the outcome 210010. Alerts can be configured based on defined parameters for clinical trials and can send automated tasks in the case of a patient or patients falling outside defined parameters 210020. Results can have predefined thresholds to trigger alerts and/or automated tasks 210030 and procedures, diagnostics 210040, or lack thereof, can also trigger an alert or automated task. Additionally, required actions such as a diagnostic test 210040 can be required within a specific amount of time. It should be noted that the ability to preconfigure rules, alerts, notifications, and tasks, in accordance with clinical trials is an example which can be applied across multiple areas of research and healthcare. The inclusion of financial decision support also expands this functionality to be of interest to pharmacological, insurance, and other entities and organization to denote key requirements for said entities, whether for a single patient, group of patients, group or groups of patients within specific parameters.

In some embodiments of the present principles, if a patient misses an appointment, an auto task can be generated to alert a user/medical care provider that the patient missed the appointment so that another appointment can be scheduled, or to the Clinical trial coordinator if it is part of a research protocol. Parameters of when to create a task, such as two missed appointments in a row, can be set. This enables automatic tracking and enables a user/medical care provider to set parameters knowing the unique individual issues with a patient. A user can determine how important a missed appointment might be for a particular patient at a glance by showing previous data projected in the background, or through one user interface on another monitor the user/medical care provider can cross check and individualize the alerts and tasks since a single missed appointment can be serious for one patient, but not so serious for another.

FIGS. 50A and 50B depicts three different representations of an intelligent alert configuration system overlayed upon several different aspects of an application in accordance with an embodiment of the present principles. The alert configuration system of FIGS. 50A and 50B can, in the case of a procedure 21510, display parameters associated with procedures, patients with like procedures, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. In the case of launching the intelligent alert configuration from a result 21520, a different set of parameters can be specified relevant to that result, patients with similar results, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. An example of required actions 21530 denotes several key actions which can occur upon triggering the alert. These include, but are not limited to, a visual or audio alert, tiers of alerts based on values, notifications to be sent and to whom reminders are to be sent. In the case of launching the intelligent alert configuration from a medication 21540, a different set of parameters can be specified relevant to medications, patients with similar medications, correlations to diagnoses, financial status, risk factors, results, or other relevant criteria, while enabling exclusion criteria and actions to be taken, while also being able to be assigned to a patient, group of patients, all patients, or a patient or group of patients with specified criteria. An example of exclusions 21550 denotes several key actions which can exclude a patient or patients from an alert.

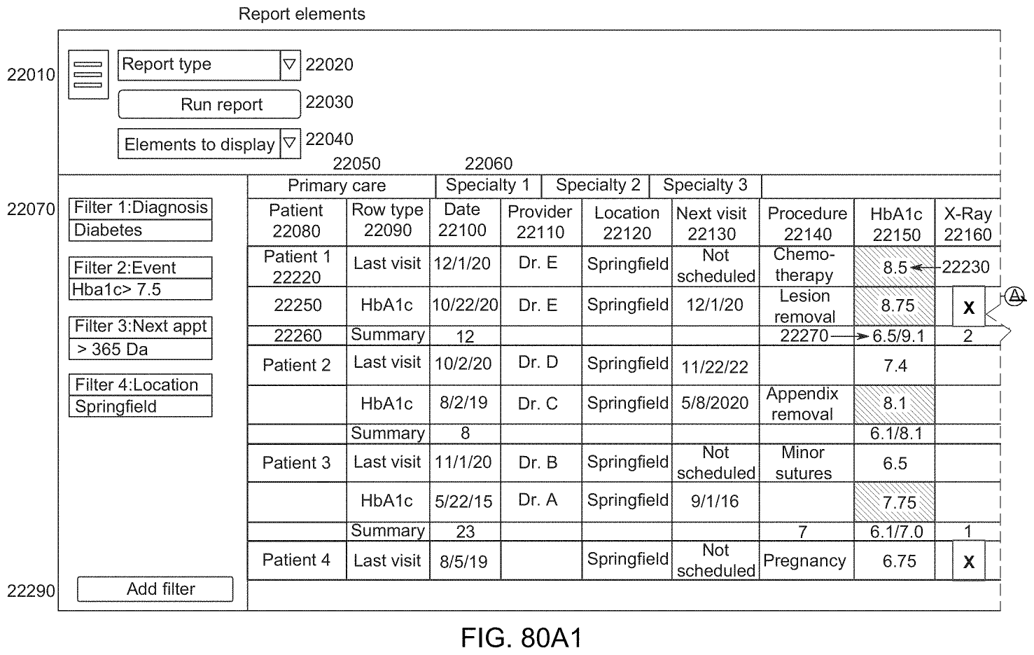

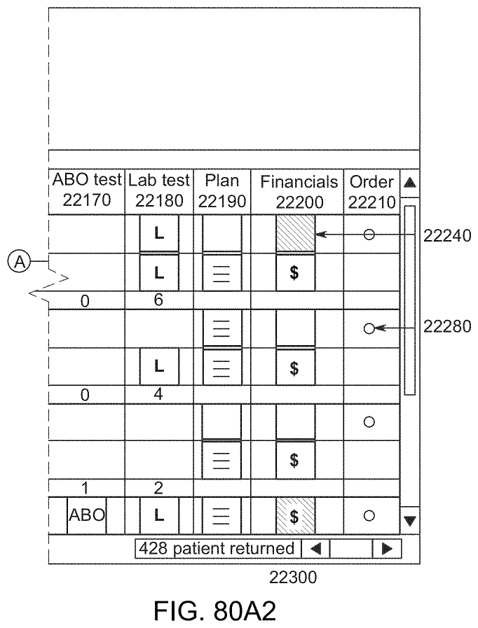

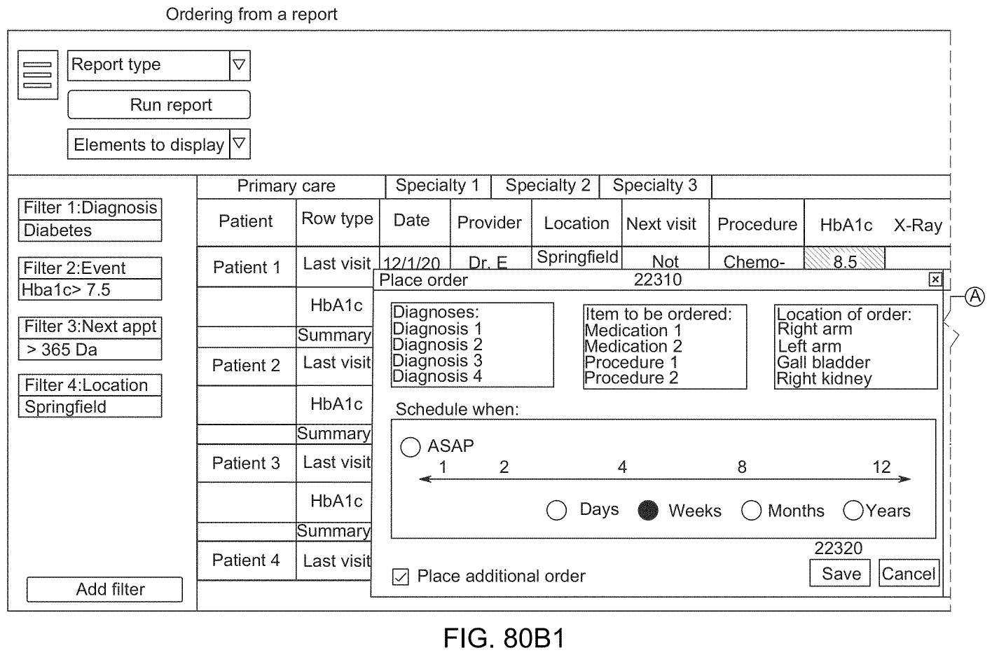

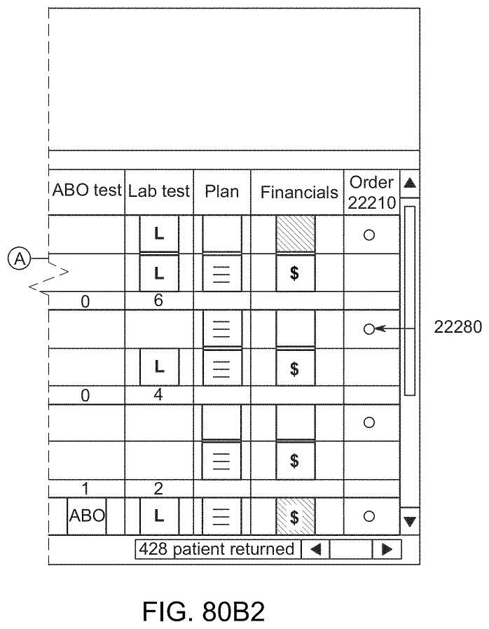

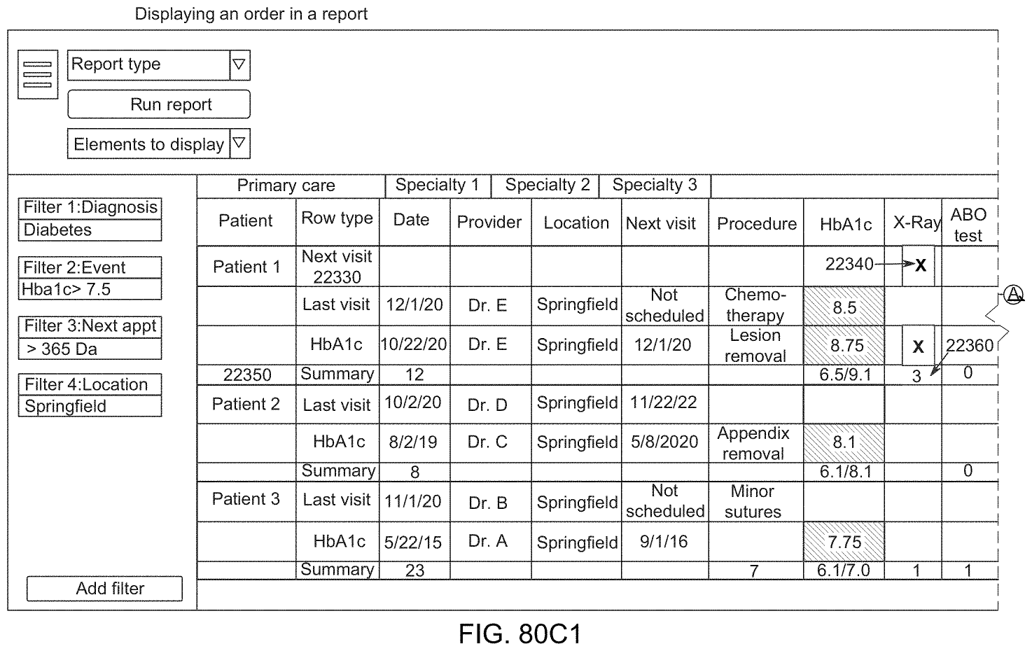

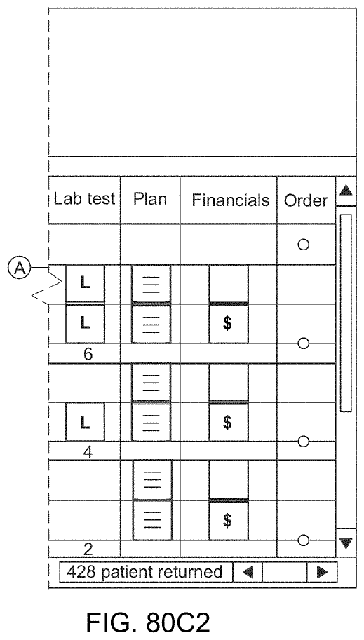

FIGS. 80A1-80C2 (collectively referred to herein as FIG. 80) depict a graphical representation of a Reporting Flowsheet in accordance with an embodiment of the present principles. In some embodiments, a Reporting feature of the present principles can be accessed through a Navigation icon 21010 of, for example, FIGS. 70A1-70A2. In the embodiment of FIG. 80, a header row exists at the top of the report, including a navigation bar 22010 whereby the user can navigate to other areas within the application, thus leaving the report display. Different report types can be selected utilizing the Report Type dropdown menu 22020. The Run Report button 22030 initiates the report process and elements to display can be selected with the Elements to Display dropdown menu 22040. As seen in this example, several rows are returned per patient as listed in the Row Type column 22090. Rows can be included or excluded using the Elements to Display dropdown menu 22040.

Columns can also be configured to display. In the example of FIG. 80, multiple tabs exist 22050-22060 denoting different configurations based on Primary Care 22050 or Medical Specialty 22060. Each tab can be individually customized or can dynamically update based on event triggers as determined by, for example the Rules Engine 10180 of FIG. 62. It should be noted however that tabs are not limited to medical specialty. In some embodiments, the tabs can be disease-specific or based on any other criteria deemed important based on use case and end user requirements.

In the embodiment of FIG. 80, a filter panel 22070 exists on the left side of the report interface. Example filters shown include a Diagnosis filter specifying Diabetes, Event Filter denoting HbA1c higher than 7.5%, Next Appointment greater than 365 days, and a Location of Springfield. Such filters are customizable and can be configured to search any medically relevant data. In the embodiment of FIG. 80, columns consist of:

Patient 22080—The patient name for the displayed rows.

Row Type 22090—The actual type of row being displayed on a line.

Date 22100—The date of the displayed encounter on that row.

Provider 22110—The provider associated with the date of service on that row.

Location 22120—The location of the encounter on that row.

Next Visit 22130—The next scheduled visit as of the date of service displayed on that row.

Procedure 22140—Any relevant medical procedures which occurred on the date of service.

HbA1c 22150—The result of a blood sugar test for the date of service on that row.

X-Ray 22160—Any X-Ray that occurred on the date of service on that row with direct access to the underlying diagnostic test imaging.

ABO Test 22170—Any blood test that occurred on the date of service on that row.

Lab Test 22180—Any laboratory test that occurred on the date of service on that row with direct access to the underlying lab test information.

Assessment and Plan 22190—Doctors notes and plan of treatment that occurred on the date of service on that row with direct access to the assessment and plan.

Financial Data 22200—Any relevant financial or billing information associated with the date of service on that row with direct access to the underlying financial data.

Order 22210—An option to place an order within the context of all relevant patient information displayed within the report.

In the embodiment of FIG. 80, on the first line of the returned results, is displayed Patient 1 22220, the last visit as denoted by the Row Type column 22090, and all relevant information to that visit. At 22230, a result in the HbA1C column has been highlighted or flagged to denote a value outside the acceptable parameters as defined by the Rules Engine 10180 of FIG. 62, and at 22240, an icon in the Financial Data column 22200 has been highlighted or flagged to denote an issue with medical billing or patient payment. Row 2 for Patient 1 22250 is the HbA1c result displayed, as can be selected in the Elements to Display dropdown menu 22040. As with the prior row, all relevant information from this date of service is displayed, as well as icons for direct access to underlying information. In Row 3, the summary row 22260, is not a date of service or encounter, but a summary of relevant information for Patient 1 22220. The Summary row can display the total number of events which occurred, such as 12 in the Date column 22100, denoting the patient has been seen 12 times, but is not limited to totals. At 22270, the highest and lowest HbA1c values are displayed, as this column summary row has been configured to show best/worst results. It should be noted that in accordance with the present principles, each column can have its own summary and ability to summarize based on preconfigured or dynamic criteria.

In FIG. 80, within the Report interface an Order column 22210 is displayed, and at 22280, an icon within the order column 22210 is displayed. The icon can represent a way to initiate the Order Screen 21370 overlain on top of the Report interface. In some embodiments, additional filters can be added using the Add Filter button 22290. In the embodiment of FIGS. 39A, 39B, and 39C, at the bottom of the report, a summary of how many patients as well as the ability to page through the result set is available at 22300. Upon selecting the icon 22280 in the Order column 22210, an order panel 22310 is displayed. Completing the order panel and selecting the Save button 22320 completes the order process. Upon committing the order, a new row displays within the report 22330 showing the next visit. In the embodiment of FIG. 80C1-80C2, the procedure ordered, in this case, an X-Ray 22340, is displayed within the row. The summary row 22350 now displays 3 X-Rays at 22360.

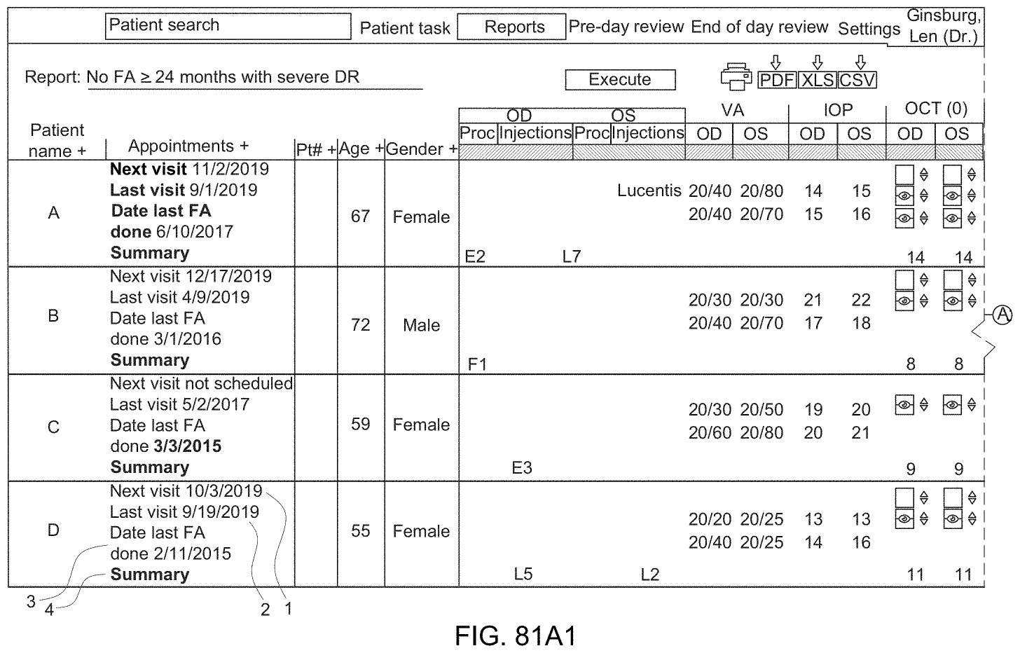

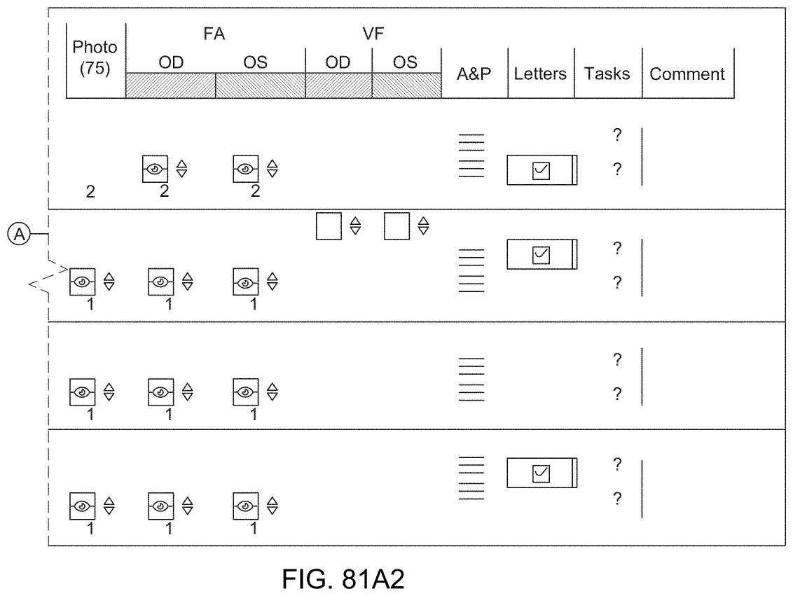

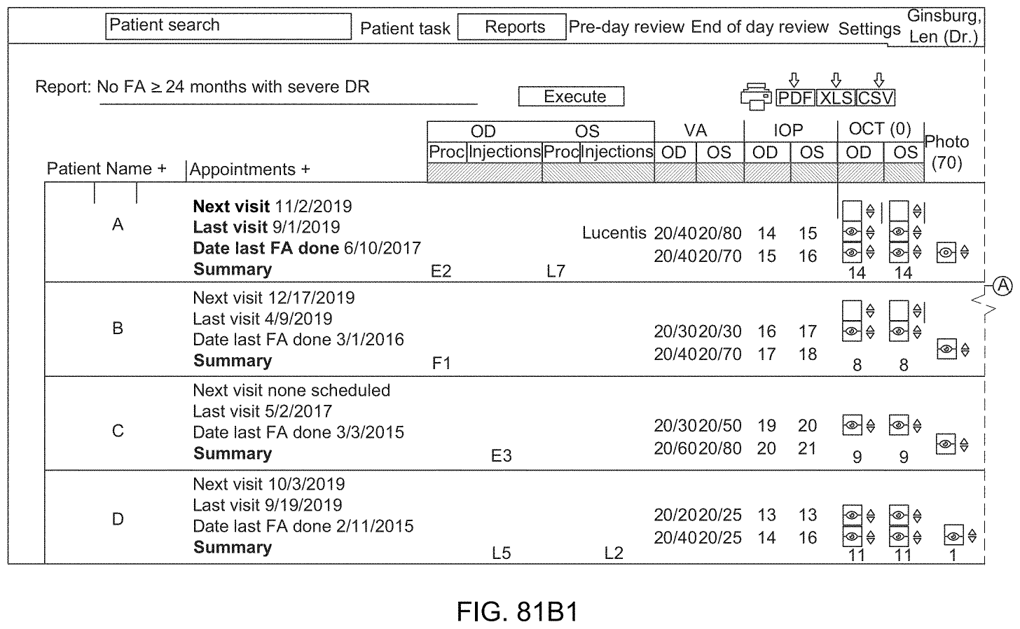

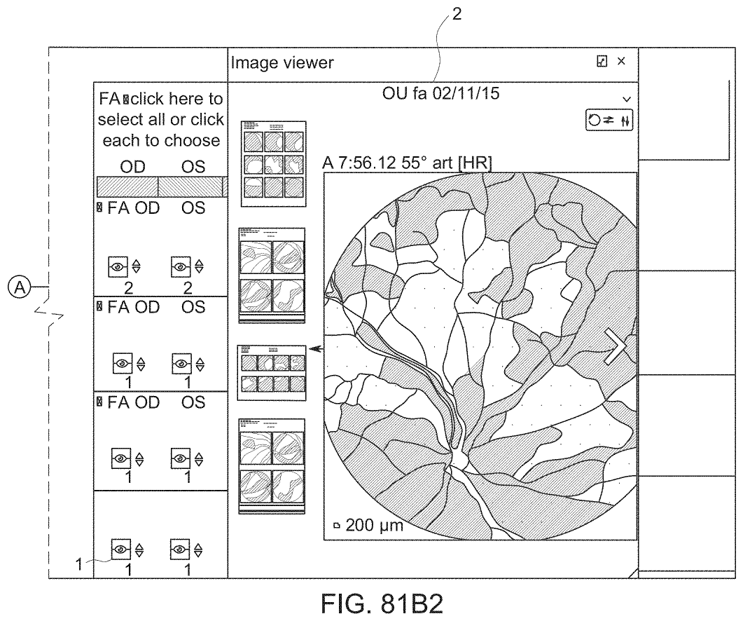

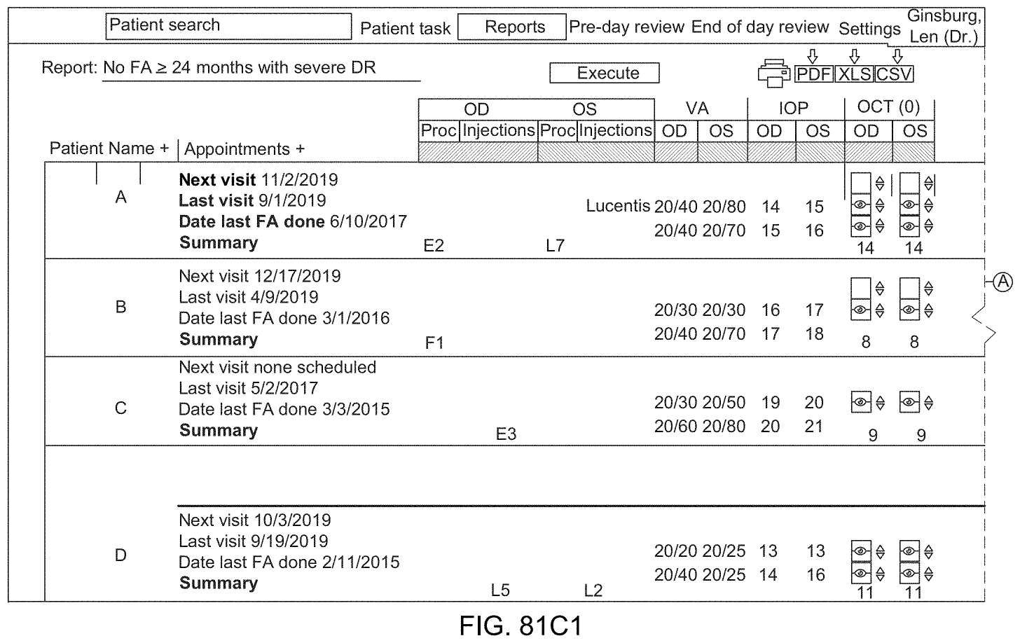

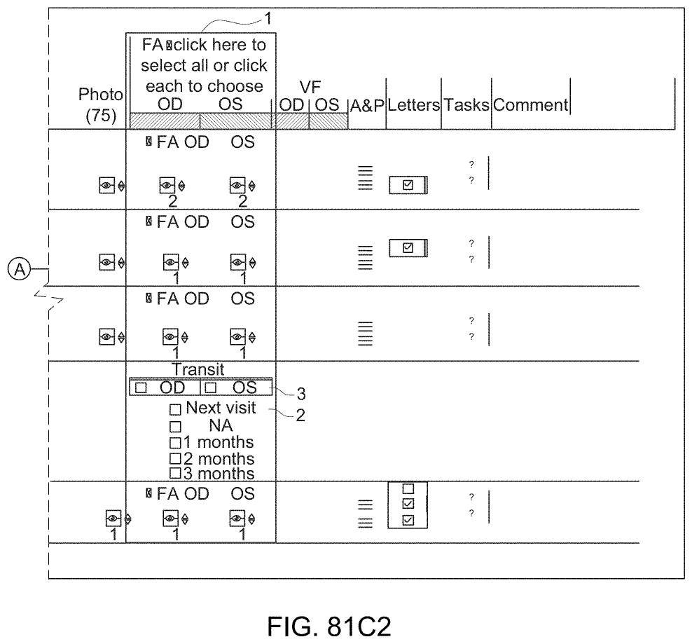

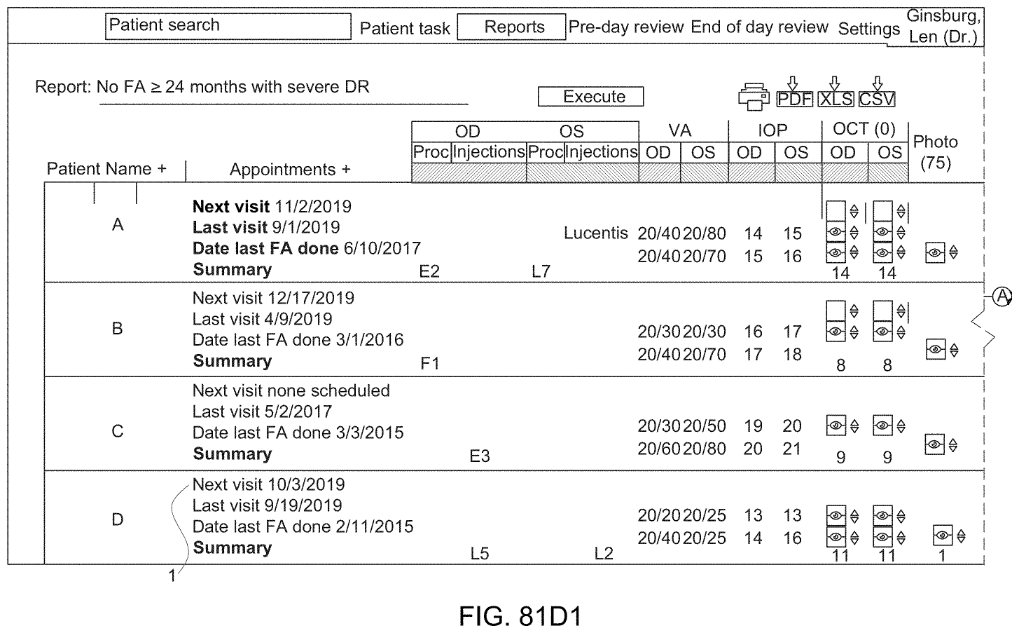

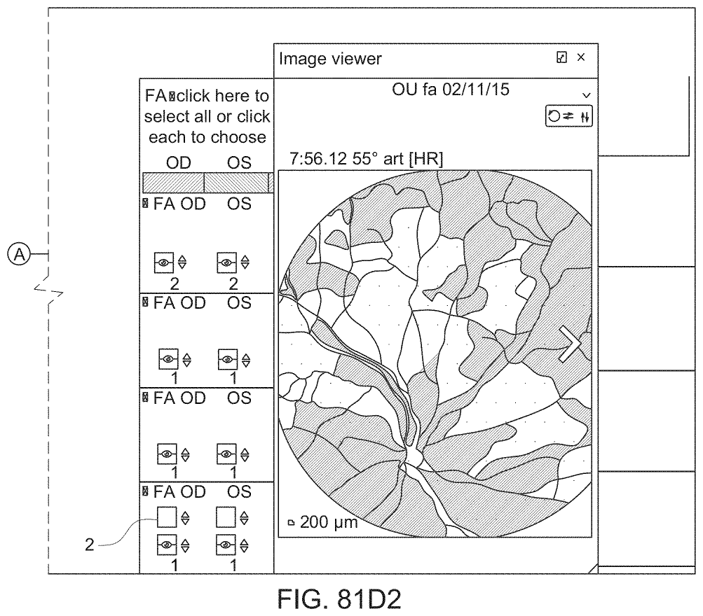

FIGS. 81A-D collectively depict a Report flowsheet that can be used for Ordering in accordance with an embodiment of the present principles. In accordance with embodiments of the present principles, a user is able to order the next visit or today's visit hit complete then go to the next visit hit complete then the next follow-up hit complete or can order all four visits and then hit confirmed at the end because as the orders are placed, a new panel pops up populating instantly as the user places orders. As such, if a mistake is made, a user can, in real time, correct the mistake because the user can see the placed orders on the panels in real time.

For example, and as depicted in FIGS. 81A1 and 81A2, a user needs to know when the next visit is (1), what the last visit was (2), and then most importantly the row that identifies the last time (3) that particular visit the diagnostic test was done. Then, the fourth is a summary row (4), so in context the user has all of the information that the user needs to decide what to do with that patient.

FIGS. 81B1 and 81B2 depict an example of how an image is now pulled up (2). The user is able to look at the fluorescein (1) from 02/01/15, on this patient, and decide if the user wants to order another fluorescein, the subject of this particular report.

FIGS. 81C1 and 81C2 depict an ordering mechanism (1) now that the user has made decisions on what to do on an individual patient since on all of these patients have a common theme could order for all of them. Now in context, the user can order the diagnostic test to be repeated “next visit” (2) or another time. The user can actually take action from the report without leaving the screen. In FIGS. 81A1 and 81A2, a user clicks on the fluorescein and the rows expand giving room to make the order of next visit, or as soon as possible, or any time period and also displays in which eye in this particular case is the first eye photographed, right (3) or left.

FIGS. 81D1 and 81D2 depict a user's selection of a test. FIGS. 81A1 and 81A2 depicts an empty box representing a fluorescein as the test has not been performed yet. When the patient shows up on that next visit, a fluorescein will be done. In accordance with the present principles, users can view reports in context, make medical decisions and then actually place orders without leaving the screen.

The embodiment of FIGS. 81A, 81B, 81C, and 81D only considers a retina Doctor, but every specialty in medicine dentistry and Veterinary medicine can have different tests and the embodiments of the present principles can be applied to each specialty. Such embodiments can show the Doctor in the column, row, panel, pop up or any other method in context that makes medical sense for that particular laboratory, pathology, radiological chemistry testing and procedures and injections of every kind.

Embodiments of a Data Command Center of the present principles can be configured for providing a Patient Evaluation Methodology included as, in at least some embodiments, a Electronic Critical Patient Reactivation (e-CPR) technique. That is, embodiments of the present principles can be configured to provide an adaptive, intelligent system for determining which patients are in critical need of care, utilizing a hybrid user-defined/automated Patient Evaluation Methodology, that can automatically take action to rectify identified issues of concern.

For example, in the ever-changing world of healthcare and healthcare IT, sorting through and identifying which data is truly of importance is key to identifying which patients are in the most urgent need of care. In order to accomplish this task, governments have mandated key data elements be recorded, stored, and shared, with the end goal of improved patient care. The downside to recording all of this data is that no human alive is capable of parsing every detail in a timely manner to make the determination as to which patients are of highest concern, and no human is capable of consuming all data points to establish unforeseen patterns of importance. Only through advancements in computing and artificial intelligence can the mass amounts of data be parsed, sorted, prioritized, and acted upon with any level of efficiency. With recent changes requiring the sharing of medical data between EHRs, there still exists no mechanism for alerting doctors or patients as to key factors which may exist in one system, but not within another. No position has ever been defined that requires a person to oversee this interrelationship of medical data, nor would a single person, or even large groups of people, be capable, in an efficient manner, to act upon such vast amounts of information quickly enough to truly manage patient care.

Electronic Critical Patient Reactivation (e-CPR) in accordance with the present principles brings to bear the full power of technological advancement and artificial intelligence to manage a task that existing Patient Reactivation Systems were previously incapable of accomplishing on their own. Through the utilization of established datasets, machine learning, and interoperability, a solution has been realized that can truly accomplish this Herculean task. A system capable of, but not limited to, identifying patients which meet the following criteria, as well as identifying previously unknown criteria, is now possible:
Meet similar Demographic criteria, even with Medical Records in disparate systems or care provided by other providers, that may also meet other key indicators for criticality.
Exhibit Risk Factors, even with Medical Records in disparate systems or care provided by other providers, that may also meet other key indicators for criticality.
Exhibit Conditions, even with Medical Records in disparate systems or care provided by other providers, that may also meet other key indicators for criticality.
Exhibit Critical Conditions, even with Medical Records in disparate systems or care provided by other providers, that may also meet other key indicators for criticality.
Have undergone Procedures, even with Medical Records in disparate systems or care provided by other providers, that may also meet other key indicators for criticality.
Have undergone Critical Procedures, even with Medical Records in disparate systems or care provided by other providers, that may also meet other key indicators for criticality.
Have Diagnostic Test Results, even with Medical Records in disparate systems or care provided by other providers, that may also meet other key indicators for criticality.
Have Critical Diagnostic Test Results, even with Medical Records in disparate systems or care provided by other providers, that may also meet other key indicators for criticality.
Are being seen by specific Specialties or Physicians, even with Medical Records in disparate systems or care provided by other providers, that may also meet other key indicators for criticality.
Have specific Appointment Types, even with Medical Records in disparate systems or care provided by other providers, that may also meet other key indicators for criticality.
Have Future Events Scheduled, even with Medical Records in disparate systems or care provided by other providers, that may also meet other key indicators for criticality.
Have a history of Canceled or Missed Appointments, even with Medical Records in disparate systems or care provided by other providers, that may also meet other key indicators for criticality.

Additionally, with the advent of machine learning, even events or categories of events not previously known can be identified utilizing an algorithm that not only identifies cause and effect but can also deduce cause by effect. In existing patient reactivation algorithms, if key factors are identified beforehand, patients may be identified which meet the criteria. e-CPR New Category Identification accounts for, but is not limited to, evaluating:
Pre-Identified Factors
Existing Data
Historical Event Factors
Current Event Factors
Future Scheduled Event Factors
Key Events
Key Results

In each of the areas described above, the evaluation is not limited to a linear parsing of data. Each step may be accounted for, but e-CPR utilizes machine learning to identify when patterns exist that were not previously identified, and automatically begins accounting for the newly acquired data. As an example, a doctor or patient reactivation system can know that a patient with diabetes needs to be seen every 1-2 years, and a patient with diabetes and heart disease may need to be seen every 6-12 months, but can be completely unaware that patients within 50 miles of a specific zip code are exhibiting complications requiring them to be seen every 3-6 months. The complications may not have yet been identified or correlated to this location, but utilizing advanced pattern analysis, e-CPR can parse all patient records and identify a pattern of worsening outcomes linked by locality. The root cause can be a factory with faulty filtration, environmental conditions, or even a localized outbreak. Such contributing factors would not have been identifiable without first connecting the data that patients with specific conditions have worsening outcomes, and that the patients exist within a certain locality. With the local population seeking healthcare at several different facilities, each facility may not notice the increased pathology due to the small sample size and focus on individual patient care. Only through correlating several factors from all locations in the area do such issues come to light.

In another example, a specific patient can have a history of relatively minor risk factors, conditions, and/or procedures, but poor compliance with maintaining a proper schedule of doctor visits. Such a patient can see 4-5 different providers, all at different locations, or can only visit a hospital for emergency care when conditions become unbearable. No one provider may be aware of the history of missed appointments because they are only missing a few appointments at each provider. In such an example, the reason for the missed appointments now becomes a higher priority. If the patient is missing key appointments with specialists they have been referred to see, such a patient is in danger of becoming critical. As mentioned, this can lead the patient to visit the emergency room for conditions that would have best been addressed through routine care. Identifying such a patient is critical, not just for individual patient care, but also for determining factors to identify similar patients before they reach this critical state, such as socio-economic conditions, language barriers, lack of transportation, and the like.

Identification is only the first step in electronic Critical Patient Reactivation. After proper identification, the most important factor is ensuring patient compliance. Existing patient reactivation systems implement many established means of communication to send a reminder to the patient to come in, by mail, email, text, and/or automated or manual phone call. In some more advanced patient reactivation systems, responses to communications may be tracked and accounted for, such as a missed phone call may be followed up on x more times, and a report can even be generated to show non-compliant patients. With e-CPR's advanced communication management, method of communication with a given patient is not limited to patient or practice preference. Similar to identifying critical patients for reactivation, an algorithm is used to determine the most effective means of communication. Historical data is compiled and analyzed, not just for the individual patient, but also correlated against other patient with similar age, conditions, locality, and other key factors, to determine that a certain patient can prefer a text message between the hours of 8 AM and 5 PM Monday through Friday, a cellular phone call between 5 and 6 PM on the same days, and 10 AM through 8 PM on weekend, but desires a home phone call outside of those times. Historical data may also point to a dramatic inability to contact a patient through established methods, thus determining a certified letter or even an in-person visit may be required to ensure said notification is delivered and received. By compiling and correlating all available data, e-CPR's AI can determine not only the most effective means of communication, but the most effective times to communicate via a specific method.

e-CPR is not limited to a single action or set of actions in response to an attempt to contact and bring a patient back in. Several steps may be predefined, but, as is the strength of e-CPR, additional steps may be defined or redefined based on current or future responses. E-CPR is also not limited to patient communication in an attempt to reactivate a critical patient. With connections throughout the care process, e-CPR has the ability to send tasks to schedulers, visually notify doctors at point of care, and even reach out directly to all of the patient's doctors, to immediately make them aware of the need for the patient to be seen for a specified reason. A process of the present principles can implement several conditions, steps, requirements, and triggers to ensure that the patient is never lost within the system. FIG. 66 depicts a Table having example parameters of a process for e-CPR in accordance with the present principles.

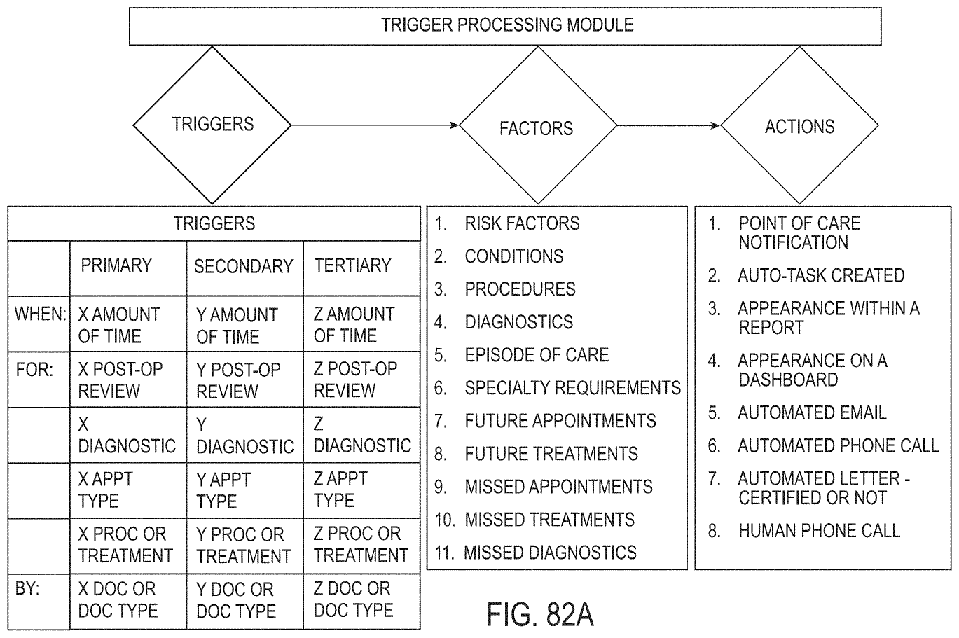

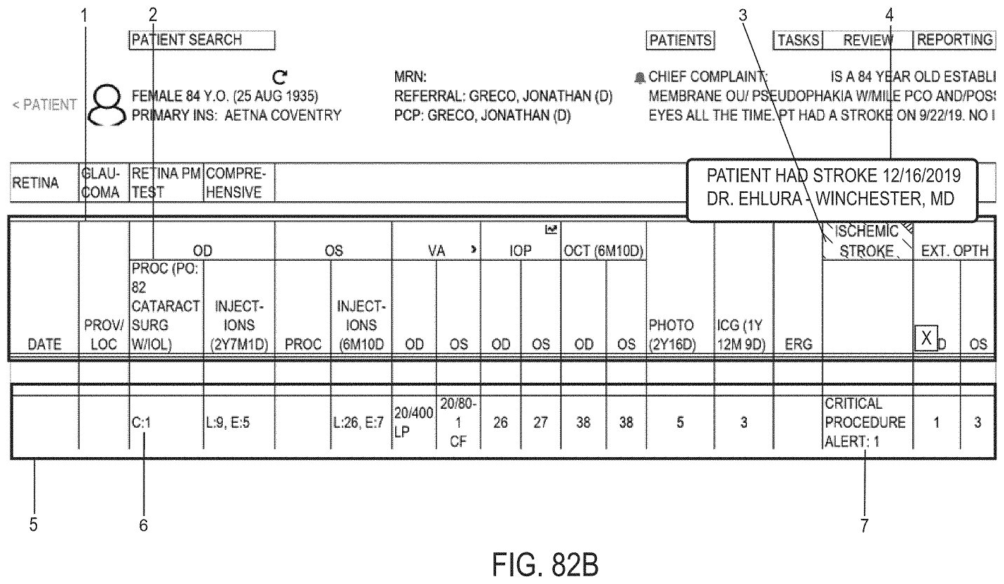

An example of such a process is depicted in FIG. 82A. While the example of FIG. 82A depicts a total of three triggers, triggers are not limited by number, nor are the steps or information contained within meant to express a limitation or linear process. Multiple and complex arrangements of triggers, counter-triggers, and multi-dimensional triggers can be implemented as needed to accomplish the requirements of electronic Critical Patient Reactivation of the present principles. It should also be noted that, in FIG. 82A, the Y-Axis display listing When, For, and By, is not limited to these values or this number of values. Pre-defined criteria, as well as criteria determined at time of execution, can add, remove, or otherwise alter this criteria list.

In the process of FIG. 82A, actions to be taken can include, but are not limited to:
Point of care notification to a Provider
Auto-Task created to a Scheduler
Appearance within a report
Appearance on a dashboard
Automated email to Patient, Practice, Provider, Scheduler
Automated phone call to Patient, Practice, Provider, Scheduler
Automate letter, Certified or not, to Patient, Practice, Provider, Scheduler
Human phone call to Patient, Practice, Provider, Scheduler
Human visit to Patient, Practice, Provider, Scheduler

As noted above, in embodiments of the present principles several key factors are compared and can affect the outcome of the critical patient reactivation workflow, either by triggering an action, or satisfying a requirement. Triggering an action occurs when a requirement is met for a trigger. For example, if a requirement for a patient with specified risk factors and conditions is not met, as in an obese diabetic patient with glaucoma not receiving a diagnostic test to track their condition within 6 months, and action may trigger a point of care notification to the specialist tracking the disease. If the requirement is not met, and a second threshold occurs, such as not receiving the diagnostic test in 18 months, an auto-task and alert on a dashboard can trigger to the schedulers to ensure the patient is scheduled for the diagnostic. A third requirement can occur if the first and second requirements are not met, which can trigger a series of notifications to the assigned specialist, and possibly all users providing care for the patient, the schedulers, and the patient, to ensure the patient is receiving proper care.

In accordance with the present principles, should additional data become available during the course of the above described process, such as a new procedure or condition being recorded, the algorithm can be reinitiated or refactored as required based on the newly acquired information. Should a patient commit to a future event, such as scheduling an appointment, which satisfies existing criteria, an additional step can be created to ensure the future commitment is met. If it is not, the patient can reenter the previous workflow, or newly defined requirements can be established.

Critical Patient Indicators of the present principles can directly interact with DHRpro. For example, FIG. 82A depicts a flow diagram of a trigger processing method in accordance with an embodiment of the present principles. As illustrated in FIG. 82B, at element 1, the DHRpro Header Row is displayed. Such a row denotes column names as well as key indicators regarding the contents of said column. At element 2, an indicator for a Cataract Surgery Post-Operative Period is clearly denoted. This indicator states that the patient is in the 82nd day of a 90-day Post-Operative Period. As such, an Ophthalmologist would clearly recognize the criticality of said patient. A column which would not normally be displayed, element 3, Ischemic Stroke, is automatically added to the Ophthalmologist's dashboard by way of Patient Evaluation Methodology, to show that said patient has had a Critical Procedure. By way of hovering over, selecting, or otherwise interacting with said column, more information may be displayed as shown in element 4, denoting the procedure, date, physician, and location where the stroke was recorded.

In FIG. 82B, element 5 denotes a DHRpro Summary Row. Such a row denotes counts, totals, min/max, assessments, best/worse, et al values based on the contents of said column. At element 6, a Count of procedures is displayed, in this case, C: 1 to denote one cataract procedure has been performed in the right eye. Finally, at element 7, a summary for the newly added column, Ischemic Stroke, is displayed denoting that a Critical Procedure Alert exists in the column. This is an example of the display of a Point of Care notification to a Provider.

Indicators can also exist within columns, within rows, outside of rows or columns, attached to modules, panels, in popups, pop outs, pop overs, and by other methods used to notify a provider. Indicators are not limited to visual indicators and may employ sound, voice recordings, and other audio methods of notification. Indicators may also include vibrational feedback and other means of notification available based on the media used to access e-CPR and/or DHRpro.

In some embodiments, a Data Command Center of the present principles is enabled to provide a Customizable, Correlative Graph (CCG). That is, the Data Command Center is able to collate data and visualize correlation between different, related datapoints, each with their own distinct visualizations. Novel to customizable visualizations is to display an array of customized visualizations correlated on a comparative axis or axes. This customized, correlative display consists of one or more visualizations of Command Center data, horizontally, vertically, on a Z axis, or on multiple axes displaying multiple events, results, and/or calculations. In some embodiments, the Customizable, Correlative Line Graph display can be launched from within a Patient Flowsheet using a button, keystroke, or series of keystrokes such as the icon shown in 21080 of FIGS. 70A1-70A2. Upon launch, the Customizable, Correlative Line Graph generates a view and can display as a popup, popover, pop out, or otherwise in relation to, but not limited by the launch point. The Graph can overlay or adjoin the underlying Flowsheet in opaque or transparent states, be pinned to the Flowsheet, and/or may hover over or aside said Flowsheet.

Upon initiating the Customizable, Correlative Graph, a series of actions are performed to determine data and format of data displayed. Preconfigured CCG displays can be stored in tables or generated at runtime based on key considerations such as those laid out in Dynamic Data Representations described above.

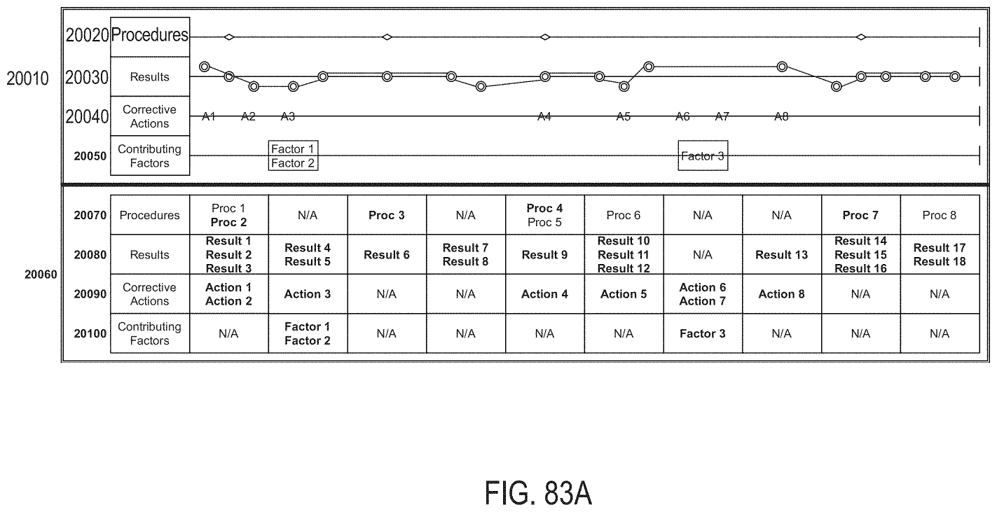

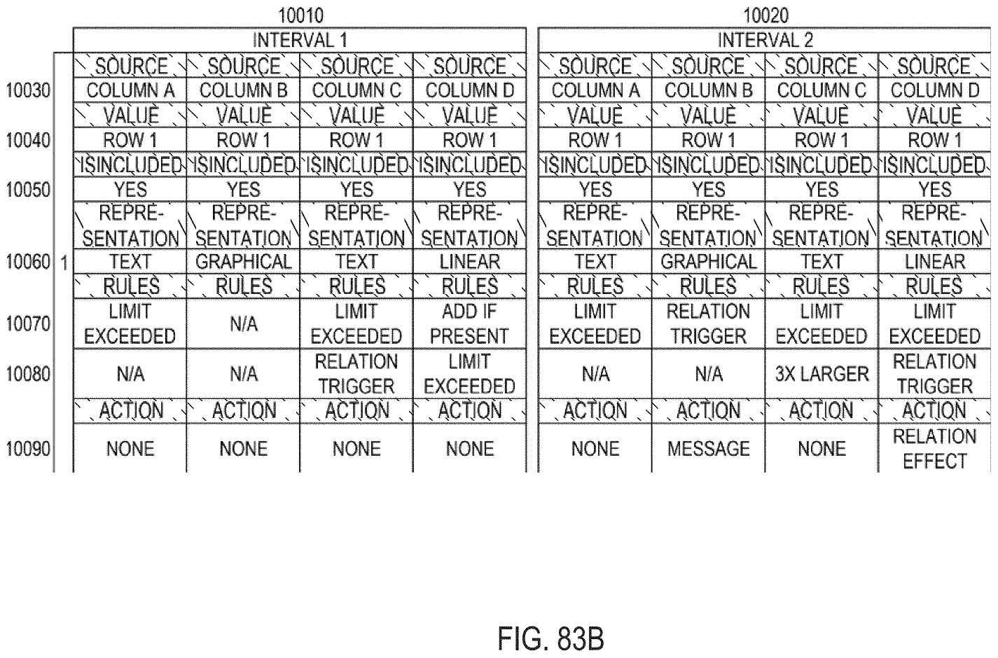

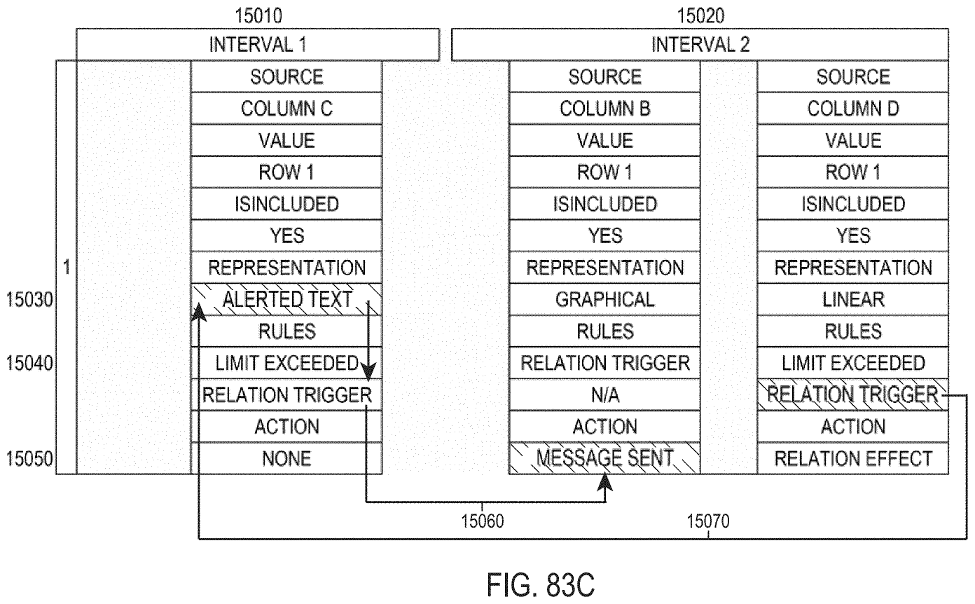

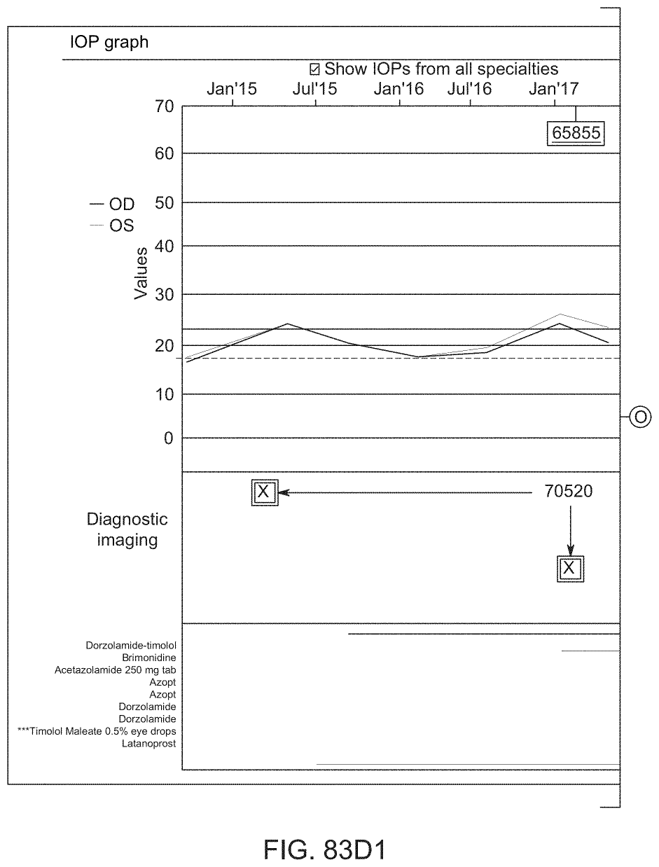

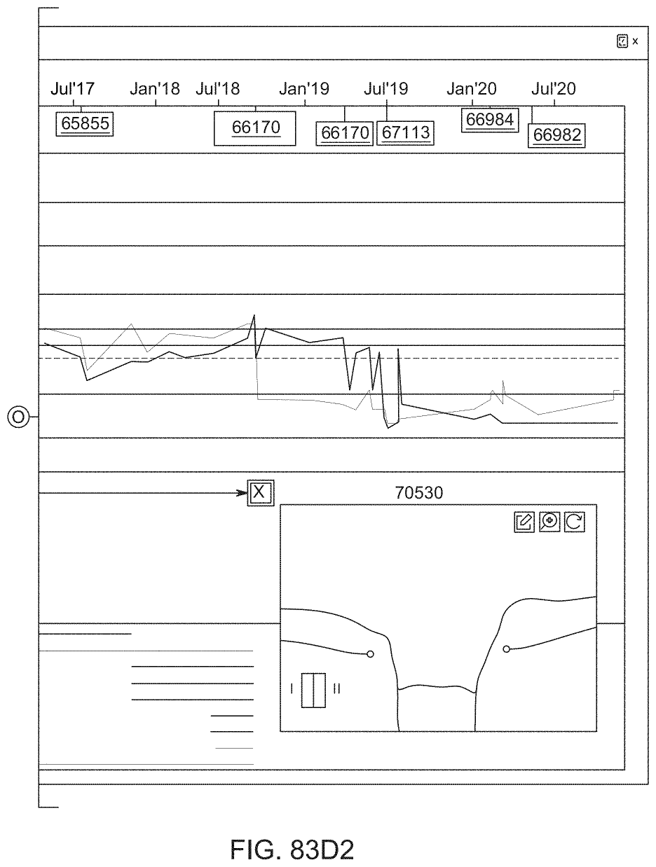

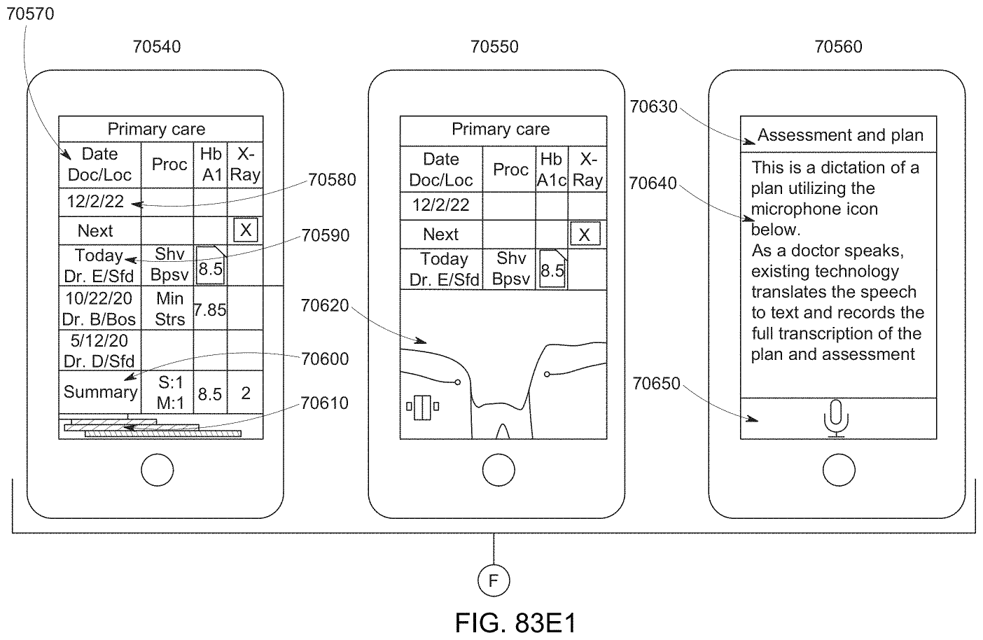

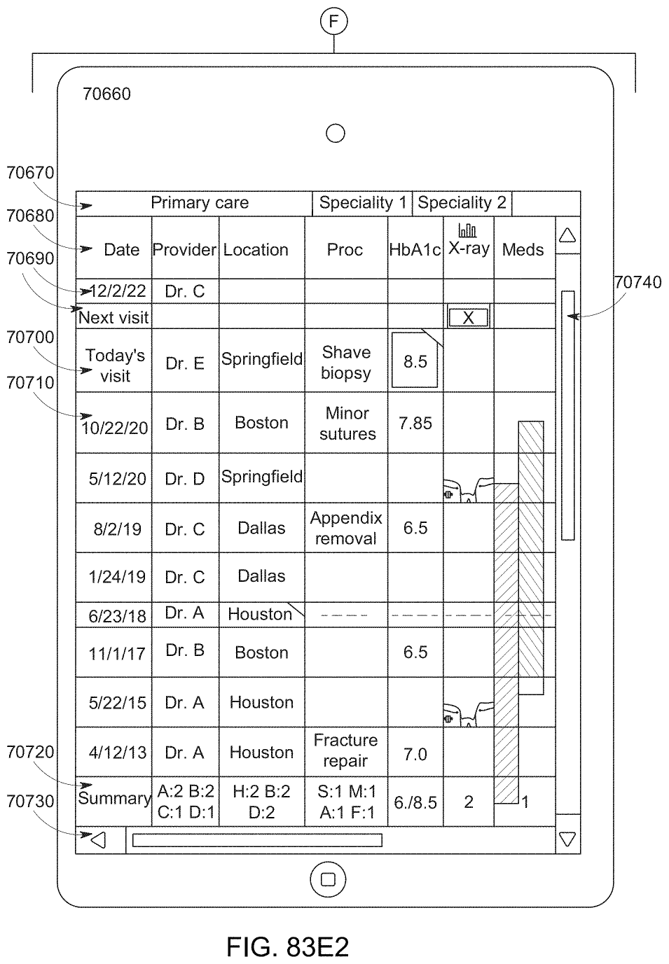

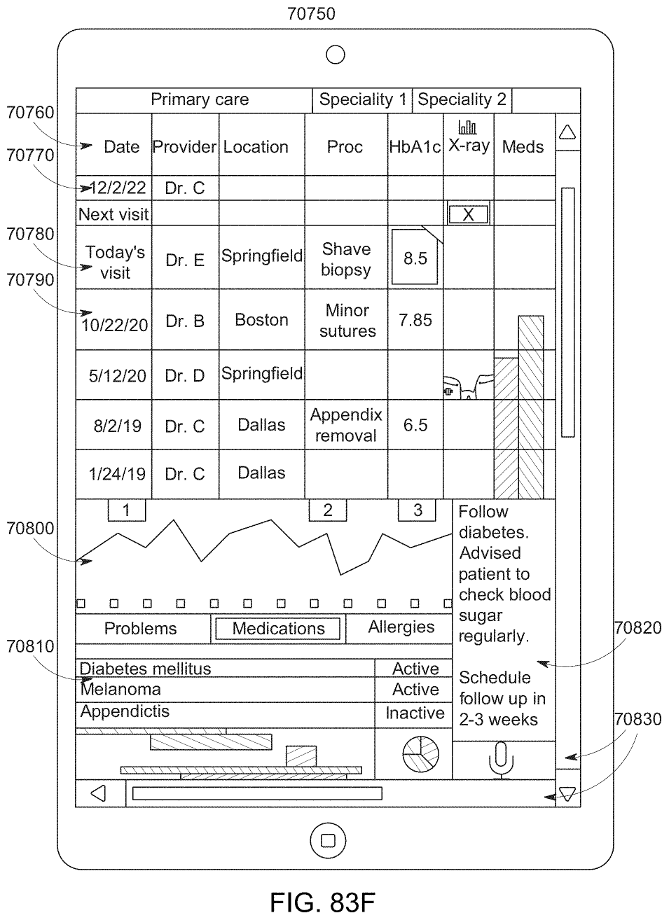

FIG. 83A illustrates a horizontal correlative graph in accordance with an embodiment of the present principles. In the embodiment of FIG. 83A, available data (20060) is visualized graphically (20010), as a series of events (20070) graphed against a timeline (20020), correlated with a series of results (20080, 20030), a series of actions (20090, 20040), and a series of contributing factors (20100, 20050). Any number of relevant details and categories of data can be correlated as needed.

FIG. 83B illustrates a series of Data Visualization Storage configurations in accordance with an embodiment of the present principles. In the embodiment of FIG. 83B, data visualization is achieved with a series of configurations to determine what and how to display. In some embodiments, Source Data (10010) consists of at least one of a Value (10030), of at least one of an Inclusion/Exclusion Rule (10050), of at least one of a Visual Representation Configuration (10060), of at least one of a set of governing Rules (10070, 10080), and of at least one of a set of Actions to be taken (10090). The data can be visualized across multiple intervals (10010, 10020).

FIG. 83C illustrates interactions between data sets in accordance with an embodiment of the present principles. In the embodiment of FIG. 83C, data stored or generated at runtime can be correlated and evaluated against other data stored or generated at runtime, reinitiating the process until no further changes occur. In such a process, data recorded for interval 1, as illustrated in 15010 of FIG. 83C, can be correlated and evaluated against data recorded for interval 2 (15020). At 15030, Alerted Text in column C is displayed, which was previously listed as Text in column C 10050 of FIG. 83B. This can be triggered by a series of actions occurring, such as a Limit Exceeded Rule invoking a Relation Trigger 15040. Such a Relation Trigger directly affects 15070 the field specified in the Relation Trigger, in this case, reevaluation of the Representation in Column C 15030 of Interval 1 15010. Subsequently, the text of Column C 15030, now invokes a Relation Trigger 15040 of its own, which directly affects 15060 the field specified in the Relation Trigger, in this case, reevaluation of the rules for Column B of Interval 2 15020. As such, a Message is now sent.

Configurations stored can be correlated between intervals, within the same interval, from different source data, and between separate categorizations of visualized data. Utilizing the methodology, one can assume any change in source data may initiate refactoring of the evaluation process, as well as any change, addition, or deletion in displayed data can initiate refactoring of the evaluation process. Furthermore, data added for future consideration, such as future appointments or orders, can be evaluated, displayed, and reevaluated.

Rendered Customizable, Correlative Graphs can be interacted with in such ways as to turn on or off represented values in a similar manner to expanding/collapsing/filtering of Dynamic Data Representations, i.e. turning on or off subsections of data, individual visualizations categorized by logical grouping, selecting only specific elements to display. Selecting data representations within the display, and/or moving elements between positions to achieve a different view, can also be affected based on principals described in FIGS. 70A1-70G2 and throughout the teachings herein. It should be noted that additional visualizations can be added, additional flags derived, and a series of rules explained throughout the teachings herein to manifest in the final rendering.

It should be noted that the single axis representation of the CSG of the present principles described above and represented in the Figures does not preclude multi-dimensional representations with multiple parallel representations as well as multiple perpendicular, or otherwise non-parallel representations.

The Customizable, Correlative Graph of the present principles reaches its logical end at which point all data is rendered, processing of rendered data has occurred, and any/all necessary actions have been taken based on the processed data, including, but not limited to, Flags, Alerts, Clinical Decision Support, and Auto-Tasks. Auto-updates to patient data can initiate refactoring of the Customizable, Correlative Graph.

FIGS. 83D1 and 83D2 illustrate direct access and parameter setting on a horizontal graph in accordance with an embodiment of the present principles. In the embodiment of FIGS. 83D1 and 83D2, at 70510, a dotted line is displayed denoting a threshold for pressure. Such a line defines the limit at which this patient, or any patient if configured globally, may reach a point at which action is required. Above this line, an event is triggered. This can result in an alert, message, task, or other notification. As the data is interactive, one can drag this line higher or lower to adjust the threshold, updating respective rules to now account for the new threshold set. Illustrated at 70520 are several Diagnostic Images. Each is positioned according to the governing timeline. At 70530, an image is shown being directly accessed from a respective icon, which generates a view of an in-context image viewer. As all data representations can be set to interactive, actions such as generating an image viewer/editor, order panel, bidirectional text editing, or other interface can be accessed.

FIGS. 83E1 and 83E2 illustrate Screen Resizing including several views of limited area displays in accordance with an embodiment of the present principles. Useful for maximizing screen space, these same principals can be applied to smaller devices such as smartphones and tables. 70540-70560 depicts how the Command Center can implement different described functionality to generate different views based on an available screen size. At 70570, concatenated text is displayed in a field to show Date, Doctor, and Location. At 70580, future events are displayed. At 70590, a Today's Visit is displayed. 70600 illustrates the Summary Row, and 70610 can be used to display medications. At 70620 of 70550, an enlarged image is displayed, while the rest of the screen shows relevant data. Direct access to edit a plan, as described herein, displays in 70560. At 70630, the type of information being edited is displayed. At 70640, text that was dictated using existing smartphone capabilities is displayed, and at 70650, the microphone icon common to most smartphones associated with text-to-speech functionality is displayed.

In FIGS. 83E1 and 83E2, 70660 represents a tablet, such as an iPad. 70670 displays specialties, 70680 is the flowsheet header row, 70690 displays future events, 70700 is Today's Visit, 70710 represents past visits, and 70720 is the summary row. In FIGS. 83E1 and 83E2, 70730 and 70740 are scrollbars used to view more information, in some embodiments off screen.

FIG. 83F illustrates another view of limited area display in accordance with an embodiment of the present principles. In FIG. 83F, a second view of a tablet 70750 is depicted. In the embodiment of FIG. 83F, data representations have been resized to display more important information. At 70760, the header row is still displayed, 70770 displays future events, 70780 displays Today's Visit, 70790 displays past visits, and now 70800 displays the horizontal graph described herein. 70810 displays the problems, medications, and allergies data representation described herein. 70820 displays the Assessment and Plan described herein. 70830 displays scroll bars to access more information, in some embodiments off screen.

As described above, a Data Command Center of the present principles is able to intelligently aggregate and display data through a variety of means. In many embodiments described within this patent, a Rules Engine 10180 of FIG. 62 determines which data to show, hide, highlight, send auto-tasks on, and other key functionality of the Data Command Center such as those laid out in Dynamic Data Representations as described above. In further embodiments of FIG. 83B described above, aggregated and filtered data can be displayed in text or graphical manner utilizing various configurations which can be stored in a table or generated during processing. Displayed data can also be aggregated into a correlative line graph (FIG. 83A) aligning multiple modules within a single interface, correlated along a common timeline.

In the Whole-Life View of the present principles, all pre-described functionality is aggregated into a single, intelligent view of a patient's whole life. All relevant data underlies the Whole-Life View, but zoom levels add an additional dimension to what is displayed. At its highest zoom level, only the most important factors are displayed. And its lowest zoom level, flowsheet-level access can be achieved. At each zoom interval, reprocessing of rules can occur to include additional data, differing representations of data, and notifications of key events.

Whole-Life View can be accessed from within the context of a Flowsheet, report, or patient list, utilizing a button, keystroke, or series of keystrokes, to initiate the Whole-Life View, such as the icon shown in 21080 of FIG. 70A. Whole-Life View can display at a preconfigured zoom level, a preferred zoom level, or can utilize rules to determine the required zoom level based on key factors that can be stored in tables or generated on-the-fly based on key considerations such as those laid out in Dynamic Data Representations, and those laid out in the Customizable, Correlative Line Graph FIG. 83A-83C. While within Whole-Life View, zooming can be achieved by selecting a button, keystroke, series of keystrokes, utilizing the mouse, hand gestures, touchscreen, or other logical means of interface. Zooming can also be automated based on rules as defined by the Rules Engine 10180 of FIG. 62, whereby important events can directly affect starting zoom level.

The determination of the importance of data to display in a Whole Life View must account for point-in-time refactoring of data displayed. While a heart attack can be hugely important, overall, if the zoom level is achieved which does not account for when the event occurred, only the events of the specified timeframe will be accounted for in the general view. Critical patient indicators can be implemented to account for events outside of the viewable display. This does not mean that events outside of the viewable timeframe are not of importance and can still affect the display of events in the view, such as the heart attack increasing the importance of a stent procedure within the view. In some embodiments, a weighting system can be implemented to make such determinations.

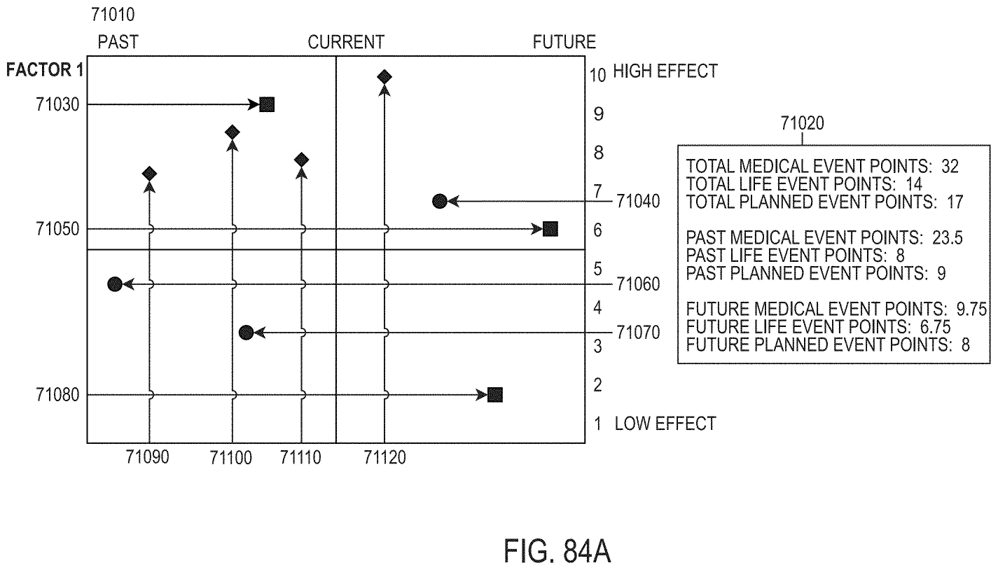

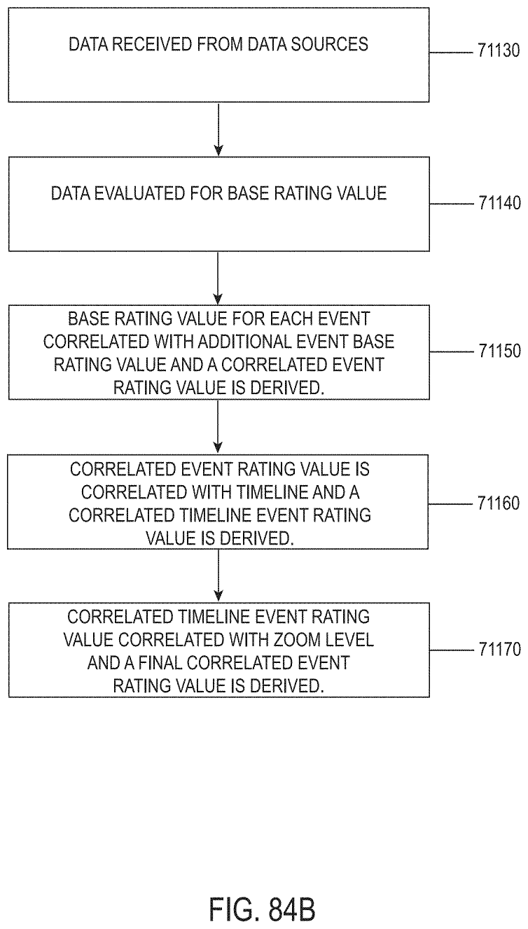

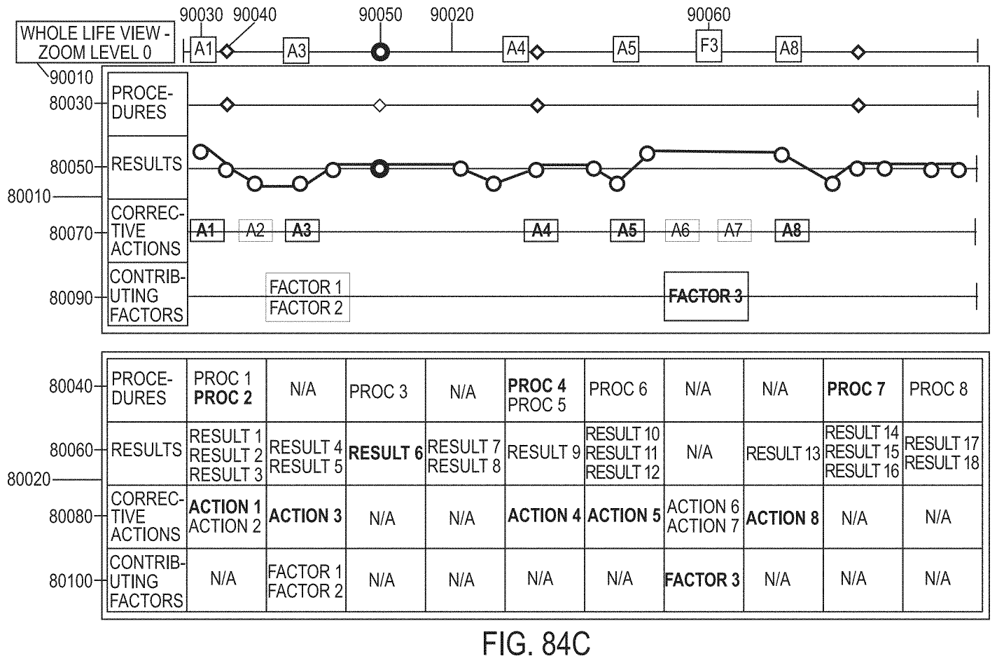

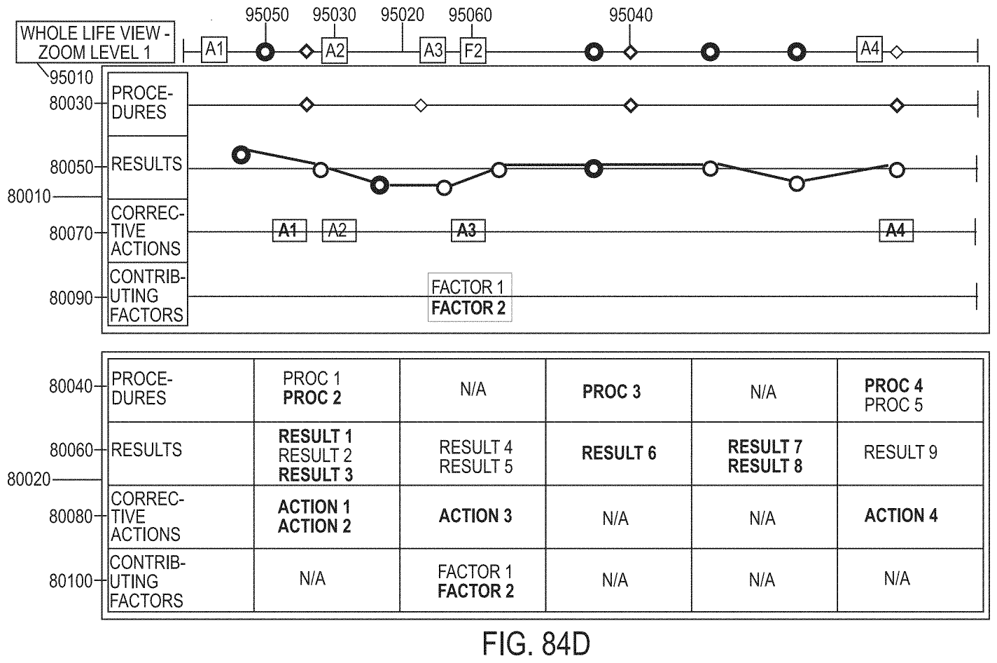

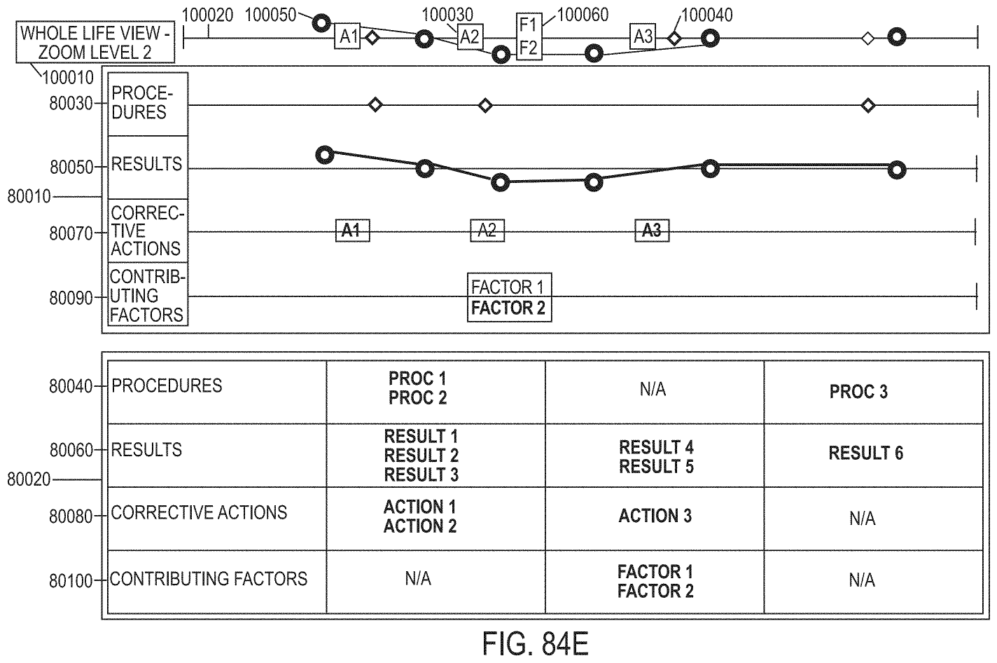

FIG. 84A illustrates a weighting system in accordance with an embodiment of the present principles. In FIG. 84A, 71010 displays a correlation between Effect (importance) and Occurrence (represented in time). 71020 illustrates an overall total of different categories of events, as well as past and future event totals. In the embodiment of FIG. 84A, weighting can occur at an overall level with a full Whole Life View displayed, or in past or present as required by zoom level, and weighting can change based on relevance of events to other events. In this example, a planned event is displayed at 71030. The planned event can be representative of a planned appointment or treatment that was not met, or the planned event can be representative of a medical event. At 71050 and 71080 future planned events are displays. Future planned events could be representative of appointments or orders scheduled for future dates. Past, present, and future planned events can interact such that a missed planned event 71030 can increase the importance of a future planned event, or medical or life events.

In FIG. 84A, at 71040, a view for a future life event is generated. This could be representative of events such as a wedding or important anniversary, which can have an effect on the weight of prior or subsequent events. 71060 and 71070 represent past life events, such as a divorce or loss of a loved one. Life events can affect the overall health and compliance of a patient, and as such are weighted and accounted for. In FIG. 84A, 71090-71110 represent past medical events, procedures, injections, surgeries, diagnostic tests, or any other medical event in the past. Each medical event is weighted and correlated with each other and other events. 71120 represents a future medical event, not a planned event, because a medical professional may know, with increased certainty, of the occurrence of this future event. Such an event can include a transplant or other major procedure for which there is little to no option for the event to not occur.

With such representations of weighting 71010 in accordance with the present principles, an axis for effect ranging from low to high, as well as past to future is enabled. This is only one example of a correlation, although many correlations work together for ultimate determination of the weight, as mentioned by the zoom level refactoring the weight of events. Total event points 71020 rise and fall based on such factors, until a final result for the specified view is determined. Results can be refactored based on changes to source data or actions taken to alter displayed data.

FIG. 84B illustrates a flow diagram of a weighting method in accordance with an embodiment of the present principles. In the embodiment of FIG. 84B, data is received from data source 71130, either external 10010-10110 of FIG. 62, internal from application storage 10160-10180 of FIG. 62 or the Rules Engine 10210 of FIG. 62. Initial data evaluation occurs and a Base Rating Value is assigned based on type and contents of data received 71140. Data evaluation ratings can be stored. Once Base Rating Values are assigned to events, all events are evaluated against each other to determine a Correlated Event Rating Value 71150. This Correlated Event Rating Value accounts for the interactions of key events and can weight one event higher or lower based on the existence of another event. Once all events are evaluated and Correlated Event Rating Values are assigned, each is then reevaluated based on when the event occurred, resulting in a Timeline Event Rating Value 71160. At each step, resulting values can be stored in volatile memory until no longer needed. Once all evaluations of and between data and timeline occur, the view is then accounted for. At 71170, the specified zoom level is factored in, and a Final Correlated Event Rating Value is used to determine the final weight of the values to display.

FIG. 84C illustrates a whole life view zoomed out in accordance with an embodiment of the present principles. In the embodiment of FIG. 84C, Zoom Level 0 (90010) is achieved and the entire timeline of the patient's life is displayed (90020). Underlying data (FIG. 80000-80020) is processed and key datapoints are intelligently determined for display. Configurations then apply to intelligently determine how to represent the datapoints. As an example, these datapoints can include all corrective actions (90030), procedures (90040), results (90050), contributing factors (90060), and any other correlated dataset display deemed necessary for display at this zoom level. Additional items of each type can also be included based on importance determined by the Rules Engine 10180 of FIG. 62, and by implementing the weighting system of FIG. 84A.

FIG. 84D illustrates a whole life view zoomed in to a first level in accordance with an embodiment of the present principles. In the embodiment of FIG. 84D, Zoom Level 1 (95010) is achieved and a subsection of the entire timeline of the patient's life (95020) is displayed in more detail. This higher level of detail can be implemented when viewing any section of the Whole Life View. Underlying data (FIG. 80000-80020) is reprocessed and additional key datapoints are intelligently determined for display. Configurations then apply to intelligently determine how to represent the datapoints (FIG. 80000-80010). These datapoints can include additional corrective actions (95030), procedures (95040), results (95050), contributing factors (95060), and any other correlated dataset display determined for display at this zoom level.

FIG. 84E illustrates a whole life view fully zoomed in in accordance with an embodiment of the present principles. In the embodiment of FIG. 84E, Zoom Level 2 (100010) is achieved and a more highly zoomed subsection of the entire timeline of the patient's life (100020) is displayed in far more detail. Underlying data (FIG. 80000-80020) is reprocessed and all datapoints are determined for display. Configurations then apply to intelligently determine how to represent the datapoints. These datapoints can include all corrective actions (100030), procedures (100040), results (100050), contributing factors (100060), and any other correlated dataset display determined for display at this zoom level, as mentioned before, or a new set of data can be defined based on the increased zoom level affecting weighting as defined in FIG. 84A. Graphical representations can take on higher levels of resolution and accuracy (100050), include predefined colors and alerts, and may allow more discrete interaction. All predefined manner of graphical and textual formatting can be displayed.

In some embodiments, a further level of Zoom can bring a user directly into a patient-specific flowsheet. Zoom can be refocused at any time, in or out, and/or on different areas of the timeline. Events on the timeline can be interacted with in such manner as would within a flowsheet, including, but not limited to, viewing images, updating plans, viewing billing data, sending a task, setting a configuration, or any other means of interaction with which that object has been defined to accept.

Embodiments of Correlative Graphs and Whole Life Displays of the present principles provide aggregating datasets into multiple modules, intelligently correlating the modules along a common axis, each with their own, unique configurations and rules, with the ability to be independently or collectively interacted with, within the context of a patient's entire lifetime of healthcare. In embodiments of the present principles, zoom levels are not limited to a set number and can accommodate all degrees of zoom levels and multi-dimensional representations with multiple parallel representations as well as multiple perpendicular, or otherwise non-parallel representations.

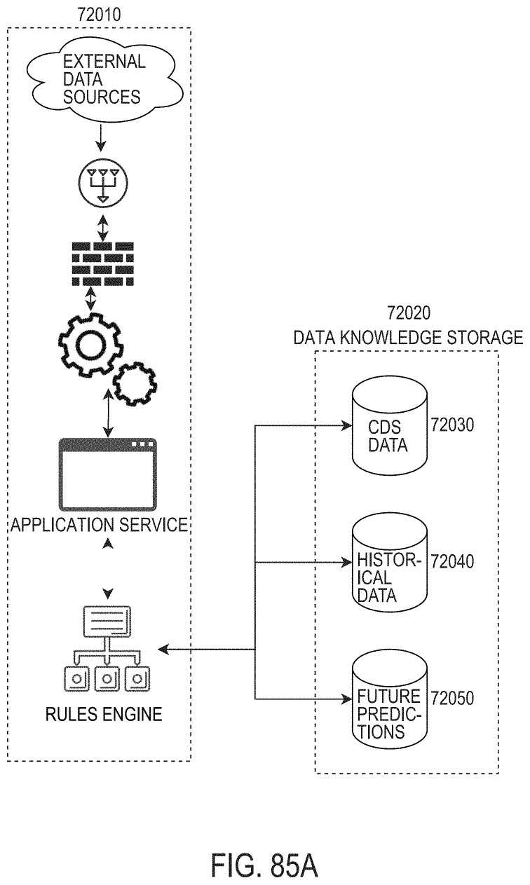

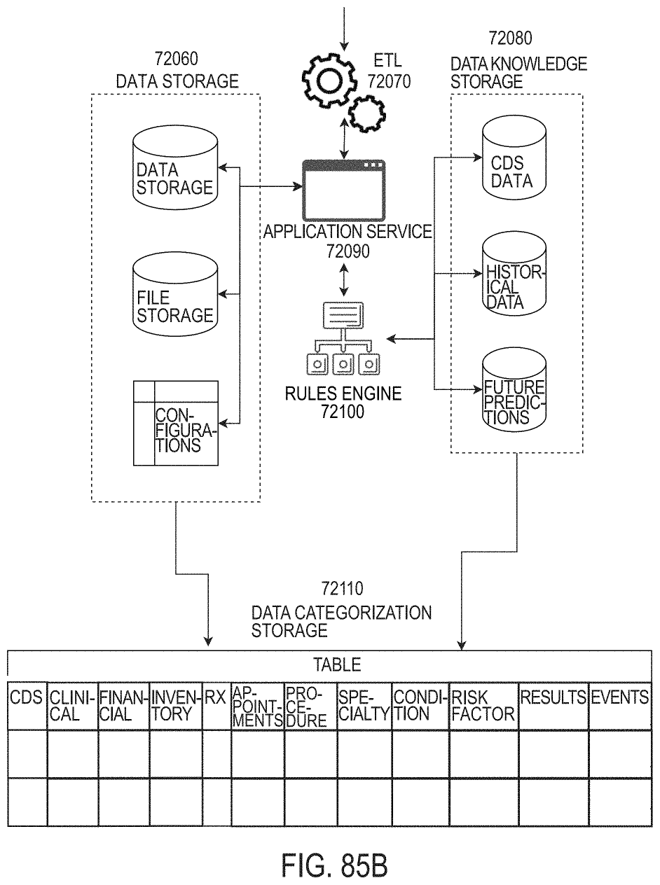

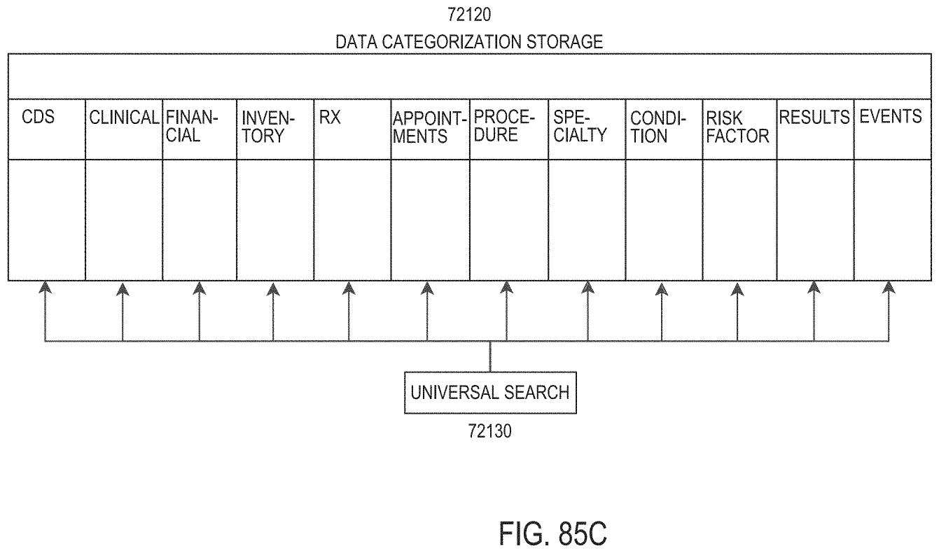

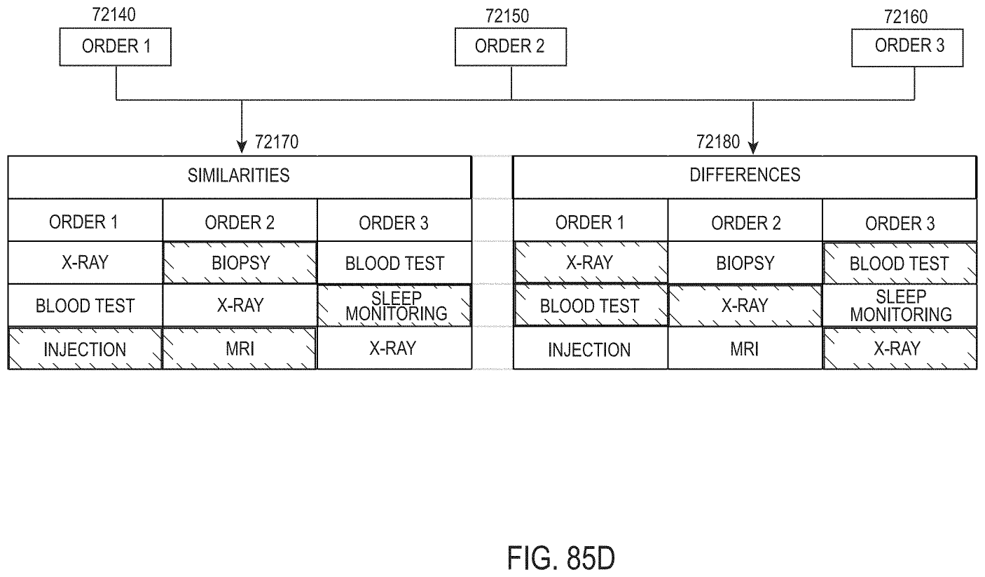

FIG. 85A illustrates a high-level data knowledge storage system in accordance with an embodiment of the present principles. The process for interaction between external data sources 10010-10110 of FIG. 62 and the Rules Engine 10210 of FIG. 62 is summarized in 72010 of FIG. 85A. As such, interaction with all available data is accessible to the Rules Engine. Subsequently, the Rules Engine can implement various storage mechanisms to maintain an accurate account of transformations and states of data. This can be achieved using, as shown in the example, Data Knowledge Storage 72020. Data Knowledge Storage can consist of separate repositories, or a single repository logically divided. Such data can be categorized, as in this example, as Clinical Decision Support data 72030, Historical Data 72040, and Future Predictions 72050. Clinical Decision Support data can be comprised of data directly accessed from External CDS Data Sources 10010 of FIG. 62, or any of the other relevant data sources, and can also be derived or compiled by the application, itself. Historical Data 72040 maintains an active record of augmentation and alteration performed by the Command Center to allow for the application to reference prior states of data at any given time. Future Prediction Data 72050 maintains an active record of predictions made for validation as well as to maintain a record of options available to correlate with actions other than those predicted for.

FIG. 85B illustrates a high-level data categorization system in accordance with an embodiment of the present principles. As previously described, data received from External Data Sources 10010-10110 of FIG. 62 is processed through an ETL process. Illustrated in FIG. 85B as 72070, this ETL process converts data received to standardized formats, which enables data of a similar category to be received from multiple sources. External data received is generally stored in the Data Storage 72060. Such data storage is managed through the Application Service 72090. Data generated by the application is generally stored in the Data Knowledge Storage 72080. Such data storage is managed through the Rules Engine 72100. All data storage is subsequently categorized for easy searchability and reporting purposes in the Data Categorization Storage 72110. Data is categorized into topics derived by requirements described herein. It should be noted that data is not limited to existing in a single category, and all data associated with a category can be stored or associated with its respective category.

FIG. 85C illustrates a high-level data category search system in accordance with an embodiment of the present principles. In embodiments of the Data Command Center of the present principles, search boxes appear to query relevant data. Each search box can be individually configured to return data only related to a specific section of the application, but all search boxes can conform to a standardized Universal Search, as illustrated in FIG. 85C. As data is received, transformed, and categorized, it is this final result of categorization by which a search is performed. The categorized information can reside in data storage such as Data Categorization Storage 72120, in a search-friendly format such as a star schema. The Universal Search box 72130 queries against this pre-categorized data, granting the ability to search data that may have originated from a variety of data sources through a single, universal search mechanism.

FIG. 85D illustrates a high-level data correlation system in accordance with an embodiment of the present principles. In embodiments of the present principles, as data is received from multiple sources, and collated from multiple practices, overlap becomes apparent. One doctor in one office may be attempting to schedule the same test or treatment as another doctor in another office. Or, perhaps, one doctor is scheduling a test or treatment that another has already completed. Such instances lead to wasted time and effort, while increasing cost. As such, when an event occurs, the Command Center searches all relevant data in the category to see if there is an existing record which would suffice the requirement. In the embodiment of FIG. 85D, three orders 72140-72160 are being placed. The Data Command Center of the present principles reviews the orders for Similarities 72170 and Differences 72180. Identified Similarities, in this example, include X-Rays and Blood Tests. It is possible the same X-Ray is required by all requestors, and as such, the Data Command Center can suggest a single order to satisfy all requirements. It is also possible that each Blood Test requires the same or slightly different requirements. The Data Command Center can suggest multiple tests be run on a single sample to reduce redundancy. In the case of the differences, 72180, the Biopsy, Sleep Monitoring, Injection, and MRI are unique. The Data Command Center can still suggest utilization of a single resource, such as one lab, for all diagnostics.

Embodiments of a Data Command Center convert and generate a display/view that efficiently translates clinical decision support (CDS) into a visual display. For instance, an OCT can be correlated in a display with injections, along with the measurements in microns of the OCT, graphing results on a timeline. The application renders efficient representations to effectively assist a user/medical care provider in determining the best course of action for a patient.

In embodiments of the present principles, once individual practice patterns are learned using AI/machine learning techniques, decisions can be customized. All users may not perform their practice in a similar manner, however, there are nationally set guidelines of preferred practice patterns based on condition. Based on these and other relevant information, sets of preferred practices can be programmed to guide users/medical care providers. Through evaluating local, regional, and national practice patterns, best practice can be identified and disseminated among users of embodiments of the present principles.

Although embodiments of the present principles can be applied to all fields of medicine where there are different medications and procedure options for treating any medical condition, described below is an example of a retina surgeon using embodiments of the present principles to treat Diabetic Macular Edema. Information always considered important to the treatment of Diabetic Macular Edema include, but is not limited to:

1. Type of injected medication in an eye

2. Frequency and time interval between injections

3. Impact on the swelling of the very center of the retina, called the fovea, as measured by an OCT; this center thickening measurement is called central macular thickness (CMT)

4. Clinical findings and how this impacts the patient's vision.

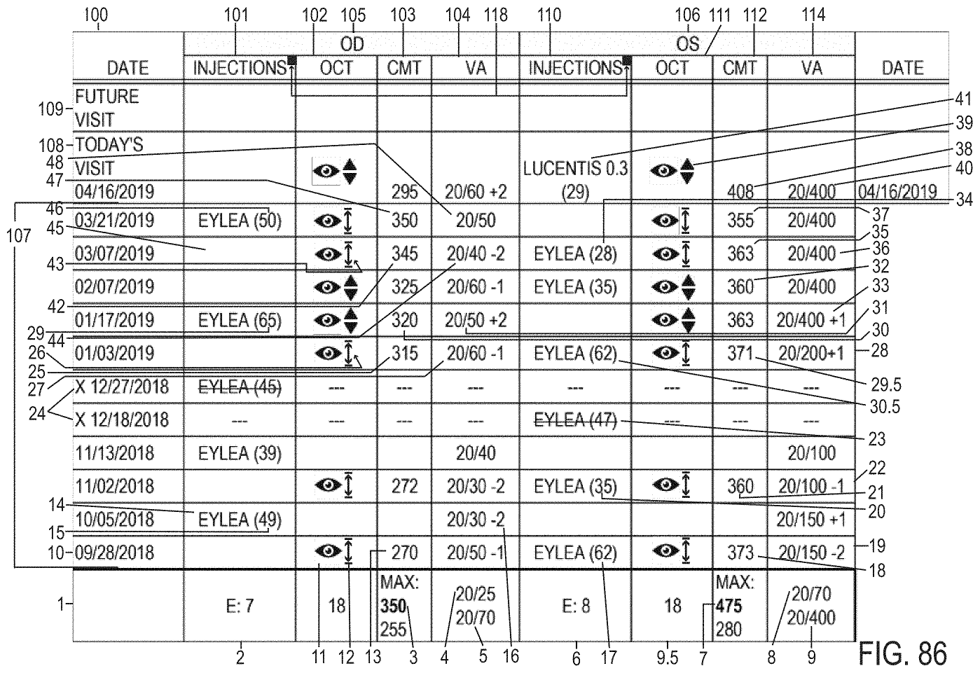

FIG. 86 illustrates a Flowsheet that can be used in predictive analytics in accordance with an embodiment of the present principles. In the embodiment of FIG. 86, 100 is the time of any medical service delivered. 101 is any medication or procedure, such as a laser, surgical intervention or any action taken at any time or date performed at a particular time 100. 102 is any diagnostic test, laboratory resting, imaging or other recording that can be measured to follow the efficacy of any action in 101. 104 is any clinical measurement of any date that can be recorded, such as any vital signs, blood pressure, symptoms, such as a level of pain indicator. Displayed is a measurement of the patient's vision. 103 is the measurement of the thickness of the retina at the center called central macular thickness (CMT). 101 shows the type of medicine that is injected in the eye. 104 represents clinical findings, 103 represents the result of a diagnostic tests, and 101 represents the type of procedure that a doctor would want to review over time. In this example, 101-105 are all right side of the body. 106 and 110-114 represent the same data as 101 to 105, but for the left side of body. It should be noted that other divisions than those shown can be used to correlate to locations on, for example, a patient's body. In FIG. 86, 118 depicts a mechanism for launching a different display, as depicted in FIG. 70A as 21080 and 1 represents a flowsheet summary row.

In FIG. 86, Element 2 is the summary data representation showing the number of injections, illustratively Eyleas. In the embodiment of FIG. 86 there are nine encounters and two cancelled encounters, which are displayed at 24. Since there were seven Eylea but only four are displaying on the nine encounters displayed, it is apparent the patient received three Eyleas before the 9/25/2018 encounter row displayed 10. In the embodiment of FIG. 86, a user is able to scroll and find these other dates or click on the E7 at 2 and just those seven encounters can be shown. Element 6 depicts that a total of eight Eylea injections have been performed in the left eye.

In the embodiment of FIG. 86, 7 shows the worst retina swelling in the left eye in a first color (e.g., red) that measures 475 microns, and the least amount of thickness is 280 microns shown in a second color (e.g., green). It can be determined from FIG. 86 that since 280 is greater than the normal 250, the patient must have presented to the practice with some level of retinal swelling. In FIG. 86, 8 represents the clinical measurement of vision of the left eye and supports the fact that the patient must have presented with some edema because 9 shows the patient's worst vision was 20/400, but 8 shows the best was 20/70, which means on presentation the patient did not see well.

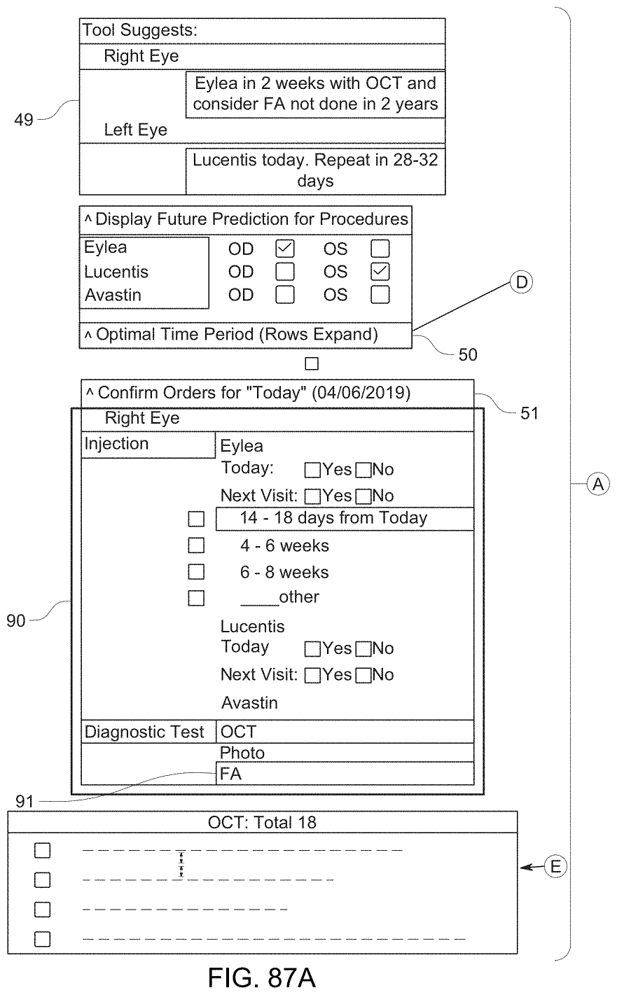

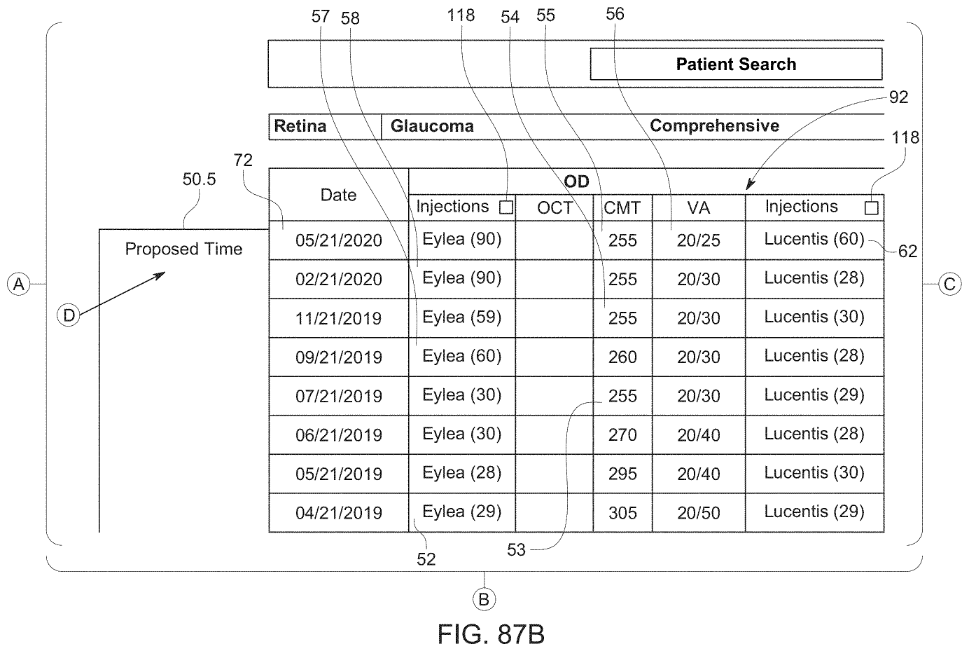

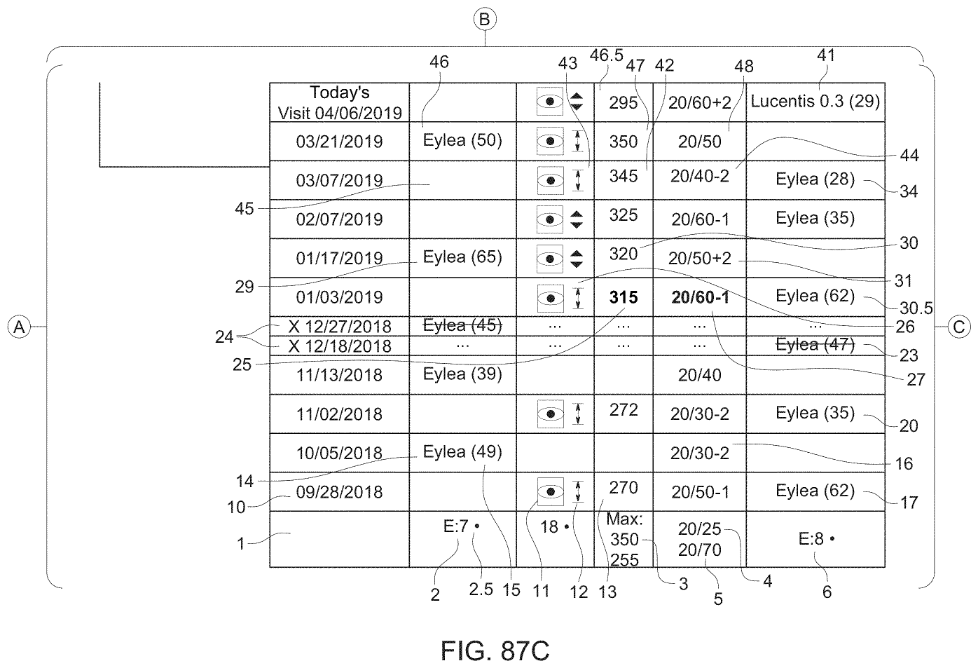

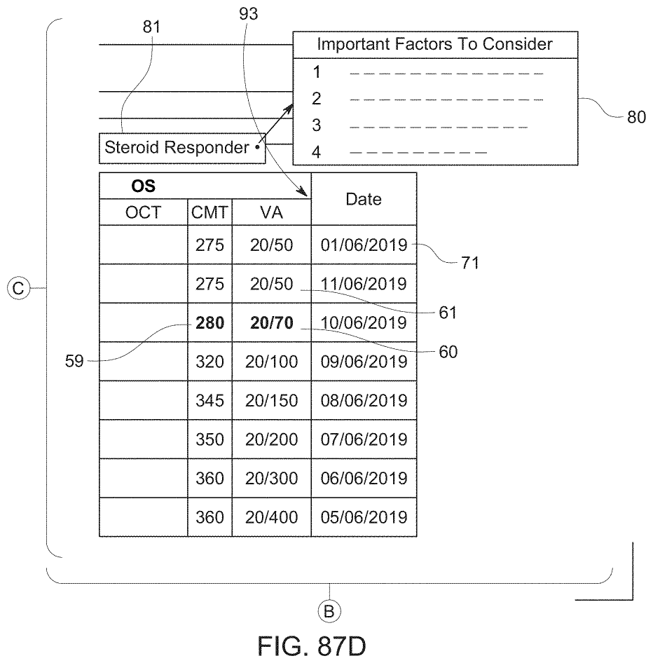

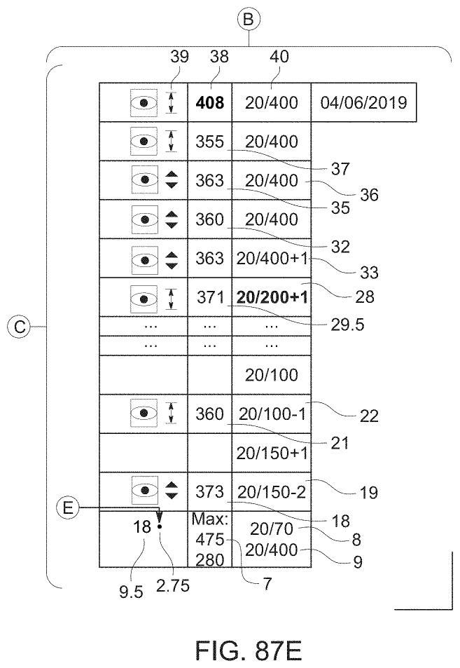

FIGS. 87A, 887B, 87C, 87D, and 87E illustrate a second Flowsheet that can be used in predictive analytics in accordance with an embodiment of the present principles. In the embodiment of FIGS. 87A, 887B, 87C, 87D, and 87E, 81 shows steroid responder in the Clinical Decision Support module and a user/medical care provider can utilize this to see an enlarge view 80. Embodiments of Data Command Center of the present principles are able to search data from all sources on a particular patient as described in FIG. 85C.

For instance, social history, 5 described in greater detail below, displays information about the patient being a smoker and can impact how the patient can respond to the medicine. Knowing what stage diabetes the patient is in, 6 of FIG. 85B, and can be evaluated against diagnostic tests such as evaluating a photograph 6.5 of FIG. 85B. The photo was 27 months ago, hence why in a photograph may be suggested for order 6.75 of FIG. 85B. The fact that a patient missed seven appointments, 7 of FIG. 85B, might explain why a particular appointment is not effective. 3 of FIG. 85B may represent insurance authorization is required, which is critical since each drug can be $2000.00 and insurance of that patient may require authorization before payment is approved.

In FIG. 86, 10 shows the first date of service and 11 is an icon for an image. By interacting with 11, the underlying image is displayed. If the presented data measuring the image 11 as seen in the next column 13, 270 microns, is not enough information for the user/medical care provider to come to a conclusion, the image can be further inspected. Each image of an OCT often has 18-30 slices. Slices can be evaluated for the best/worst thickness of that particular slice of the retina. There might be a particular slice that presents abnormal findings, and as such can be weighted to be alerted or to display as the first image in accordance with the present principles.

In FIG. 86, 12 is a symbol that can have many gradations. Ranking in any method the importance of the improvement, no improvement or worsening of an OCT. Such a scale can be utilized to display the underlying data as improving or worsening. 14 shows an Eylea injection was performed on this date of 10/5/2018 and 15 shows the number of days since the last injection was given, which was 49 days. On 10/5/2018 no OCT was performed. This makes sense because only seven days earlier on 9/28/2018 11 an OCT was performed 11 so there was no need to repeat. There are governing rules for how often an insurance companies will pay for such a test. They usually will not pay under 4 weeks, which is 28 days, which also happens to be the earliest time period that insurance will pay for the specific type of injection associated with this disease. 16 shows that the patient's vision is 20/30, an improvement from the prior visit.

In FIG. 86, 17 shows that in the left eye, Eylea was injected, and it has been 62 days since the last one. 18 shows that the CMT is 373 microns, which is about the mid-point to what the minimum and maximum is as seen in 7. 19 shows that the patient's vision is 20/150. On September 28th, the doctor decided to inject the left eye 17. On October 5th, instead of waiting 62 days 17, since the patient's vision remains decreased at 20/150 19, but we know the best vision of the patient was at one time 20/70 8, the doctor decided to do it more frequently. Therefore, in 20 it is visible that the Eylea is repeated 35 days after the last injection.

In FIG. 86, 21 shows that there has been a little improvement to 360 microns from 18, 373 microns, and that the vision has also slightly improved to 20/100, as seen by comparing the value in 22 to the value in 19. It was then decided on 11/2/2018 to repeat the injection in the right eye 20 and for the next visit, 11/13/2018, which falls in a relatively safe parameter of just 39 days after the previous, to inject the right eye. Sometimes, if the patient does not worsen by spreading out the injections, perhaps stopping injections all together can occur. However, in the embodiment of FIG. 86, on 11/13/2018, the patient is scheduled for an Eylea injection on December 18th for the left eye 23, which is 47 days after the previous left eye was injected.

Depicted in FIG. 86 is an example where on December 18th, the patient has a cancelled visit, 26. In such instances, embodiments of the present principles can now present a notification such as, “This patient has already fallen out of parameters of waiting too long. You must call them immediately to get them in ASAP.” In FIG. 86, at 24, it can be seen that on December 27th the patient had previous scheduled to have Eylea in the right eye, which would have been 45 days after the previous injection of 11/13/2018. 26 depicts that on January 26 an indicator that the OCT is worse. In FIG. 86, 25 depicts that the thickness is 315 microns. Compare that to the visit on 11/2/2018, which was 272 microns and there is a dramatic change in thickness. This is why the alert is displayed. In some embodiments a trigger can be set at a specific percentage change between measurements to alert a display.

In FIG. 86, 27 depicts the 20/60-1 in color (e.g., orange). The reason, the patient has gone from 20/40 on 11/13/2018 and has now decreased their vision by 50% to 20/60-1, is perhaps because of the two cancellations. In some embodiment, 24 can be highlighted (e.g., lit up) to help explain the conclusion. In FIG. 86, at 28 the patient is 20/200+1 OS, which is legal blindness. Such a result can be presented as a more critical alert because there has been a doubling of this patient's result.

In FIG. 86, 29.5 shows 371 microns of thickness compared to 21 at 360 microns. It is not highlighted because the 11 points, percentage wise, is not so much of a drop compared to the right eye that went from 272 to 315 at 25. 30.5 depicts that the doctor chose to do an Eylea injection in the left eye, which was 62 days from the last injection. From FIG. 86, it can be noted that 1/3/2019 was 52 days from the last time an Eylea injection was done in the right eye. The cancelled visit on 12/27/18 shows if not cancelled, the Eylea injection would have been 45 days since the last time, and on 1/3/19 is seven days later. Therefore, if the Eylea injection was performed in the right eye, it would have already been 52 days since the last injection. In some embodiments, 52 days can be displayed at 26, reminding the doctor to take action. In the embodiment of FIG. 86, however, the doctor decided to treat the left eye perhaps, because it had been a significant amount of time since last injection, 62 days shown at 30.5.

From FIG. 86 it can be noted that this patient already had missed appointments, which might be an example of a circumstance in which embodiments of the present principles can suggest injections in both eyes, due to poor patient compliance. In FIG. 86, it can be noted that in 24 and 23, the Eylea was scheduled first in the left and on December 27th it was the right eye. In FIG. 86, at 29, it is noted that the patient returned on 1/17/2019. That is a full two weeks later. As is noted, that is already 65 days after the last injection 26. In FIG. 86, it can be noted that the patient's thickening is continuing to worsen at 320 microns 30.

In FIG. 86, at 31 it can be noted that the vision is slightly better, yet 28 shows legal blindness in the left eye. On 1/17/19, the doctor gets the patient scheduled in 20 days on 2/7/2019. In FIG. 86, because while 2/7/2019 represents 35 days since an injection in the right eye, and 32 shows 360 microns and 33 shows 20/40 vision, it is reasonably good that this left eye is being injected at 35 days. On February 7th, it has only been 20 days since the last injection of the right eye. Therefore, on February 7th, injecting both eyes could not have been performed as insurance payments require waiting at least 28 days. Since, in FIG. 86 it can be noted that it has now been 35 days since an injection occurred in the left eye and this is a more time appropriate injection cycle and there has been a slight improvement to 360 microns 32, a switch may not yet be recommended.

In FIG. 86, 34 in the example of 3/7/2019, it is noted that the patient has returned in the precise 28 days for a repeat injection and 35 shows that there is 363 microns of thickness. This is problematic. There is an increased thickening of 3 microns from 32 when there was 360 microns and the injection occurred only 28 days ago. In FIG. 86, 36 shows no improvement in vision and the patient is now twice as bad as legal blindness. In this instance, the Eylea is being injected and measured at 28 days, which is perfect timing and Eylea seems not to be working. In some embodiment, at 34, the medication could have been switched, but perhaps for this patient it was worth trying it one more time or insurance required Eylea injection instead of another drug as permission to switch may require advanced authorization.

In FIG. 86, 30.5 depicts that the Eylea was injected at 62 days. There was worsening from the previous visit of 20, but that could have been a time factor of a delay because of the cancelled visit depicted in 24. As noted from FIG. 86, the patient does return on 3/21/2019, 37, and the OCT is performed again. From FIG. 86 it can be noted that only two weeks after the injection, a maximum drug effect and the OCT indicates that the central macular thickness has improved to 355 (48) from 363, but the vision has remained 20/400. This is not a satisfactory result. Having an improvement of just eight microns, just two weeks after an injection, embodiments of the present principles can suggest that at the next visit on 4/6/2019 there should be a switch from Eylea.

In FIG. 86, 41 shows that now there is a suggested switch to Lucentis, but 39 shows that there is even further worsening of the central macular thickness. In FIG. 86, 38 is highlighted and depicts that it is substantially worse than 355, and 40 depicts no improvement in vision. From FIG. 86, it is noticeably clear the Eylea is not working because it is only 29 days since the last time the Eylea had been injected. So, now two times in a row at 28 days and 29 days the Eylea has not only not improved the patient's condition, but the patient has been worsening. Therefore, in some embodiments a switch in medications can be automatically recommended.

In FIG. 86,43 shows a colored (e.g., red) icon alert that the OCT is getting worse. 42 depicts that the thickness of the retina has increased to 345 microns from the previous visit of 325 microns. 44 depicts that the vision has not worsened. Nevertheless, on 3/7/2019, with this being the only good eye, FIG. 86 depicts that only the left eye was injected. As depicted in FIG. 86, when the patient returns on 3/21/2019, it had been 50 days and the retina has become more swollen to 350 microns. In FIG. 86, 47 depicts a worsening of another 5 microns from the visit on 3/7/19 42 and the vision has slightly worsened to 20/50, 48.

Embodiments of the present principles described herein enable users/medical providers to visualize the impact of options for care on a patient with the assistance of the predictive analytics of the present principles. In accordance with the present principles, recommendations can be displayed in context with patient data as shown in 41 of FIG. 86 which automatically suggests Lucentis for the left eye on “todays visit.” In some embodiments, pertinent information of why a recommendation is being made is also displayed. In some embodiment, a Data Command Center of the present principles can display a future likely scenario if the user/medical care provider chooses the suggested course of action or, if the user chooses another course of action.

In FIG. 86, 41 depicts an example of a suggestion that the left eye have a Lucentis injection and repeat it every 28 to 30 days and the right eye be treated with Eylea two weeks from “today's visit,” Apr. 6, 2019.

FIGS. 87A, 887B, 87C, 87D, and 87E illustrate a second Flowsheet that can be used in predictive analytics in accordance with an embodiment of the present principles. In the embodiment of FIGS. 87A, 887B, 87C, 87D, and 87E 50 enables a user with an option to display future predictions for different drugs. An option for Eylea in the right eye and Lucentis in the left eye is depicted in FIGS. 87A, 887B, 87C, 87D, and 87E. Future encounter rows 50.5, can be added, showing what the results in the future could be. Reviewing past events for patient compliance can enable a determination of future compliance, and as such adjust projected outcomes accordingly, such as the two missed visits at 24. In FIGS. 87A, 887B, 87C, 87D, and 87E, 41 depicts that the doctor treated with Lucentis in the left eye and Lucentis is repeated every 28 to 30 days all the way through to 11/6/2019, and as such, compliance weighting can increase. In the embodiment of FIGS. 87A, 887B, 87C, 87D, and 87E, 62 depicts that the recommendation is to change from 30 to 60 day intervals for injections. Therefore, as depicted in FIGS. 87A, 887B, 87C, 87D, and 87E, by 1/6/2019, 71 depicts that the left eye has greatly improved with vision now recorded as 20/50, which 8 in the summary row depicts that before this reading in 71, the best vision was 20/70. Therefore, the data of FIGS. 87A, 887B, 87C, 87D, and 87E predicts that continued use of Lucentis would result in better vision than the patient has ever had since vision measurements have been recorded.

65 displays that Eylea is injected that day, but now 9/6/2019 65 displays 500 microns, augmented in red, which could indicate another threshold of the patient's retina becoming especially thickened at 500 and the vision is now worse than 20/400 at count fingers also being displayed in red. A mandatory switch is displayed on 65, suggesting there is likely no good reason to consider Eylea any longer and therefore suggests switching to Lucentis. Now, on 9/6/2019 Lucentis has to be prescribed even though the doctor had initially chosen Eylea over Lucentis and rejected switching to Lucentis back in #41. The display shows the future visit with Lucentis finally a slow improvement.

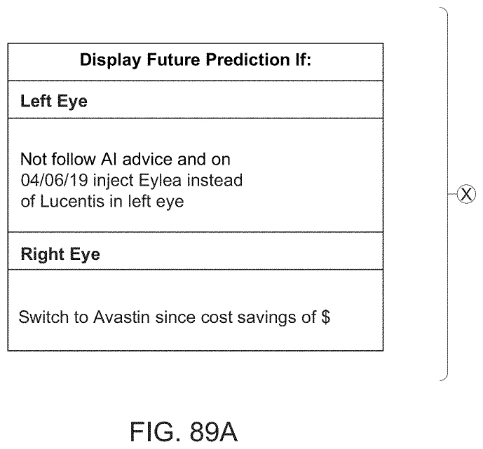

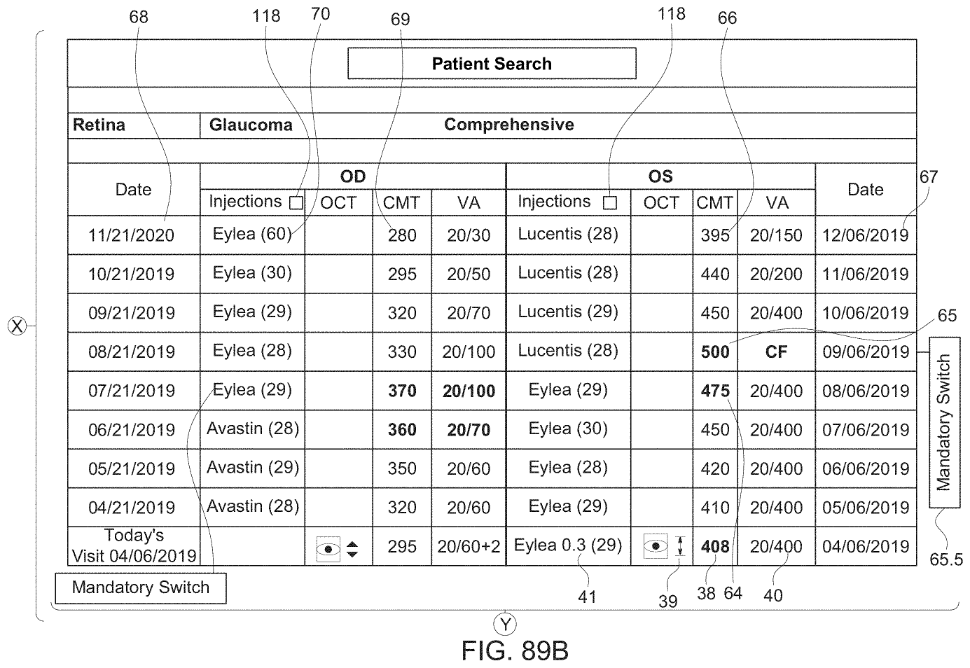

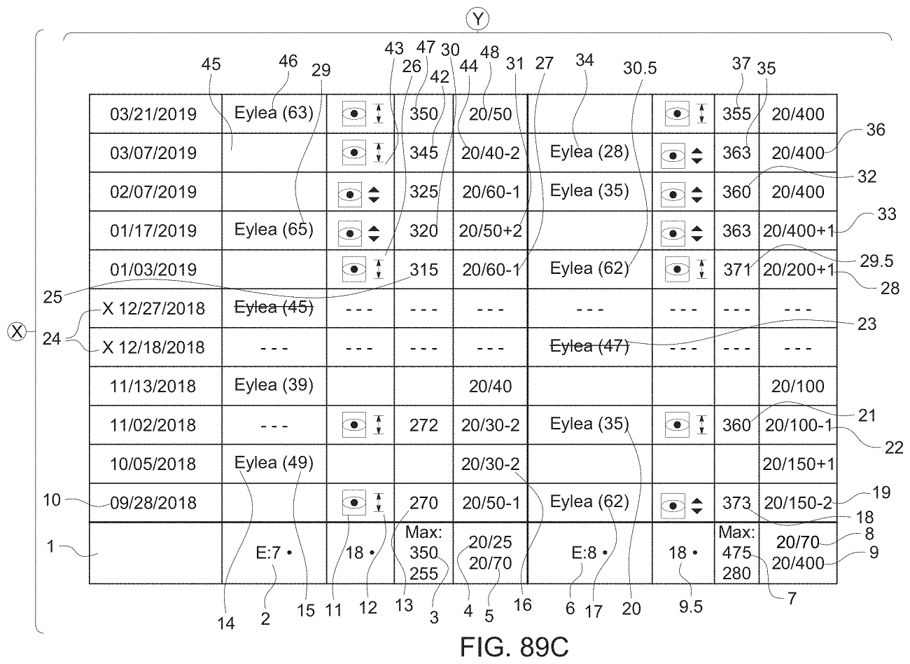

Finally, on 12/6/2019, 67, after four injections of Lucentis, approximately every 28 days, the CMT 66 is 395 and the vision has improved to 20/150. However, compare that to FIGS. 87A, 887B, 87C, 87D, and 87E, which can be toggled back and forth and displayed simultaneously or with transparency, and the doctor can visualize that the patient's vision is three times better in 71 of FIGS. 87A, 887B, 87C, 87D, and 87E, the vision is 20/50 when using Lucentis as initially proposed starting 4/6/19. The CMT 11/6/19 is 275 when using Lucentis as suggested 4/6/19 compared to on 12/6/19 of 67, FIGS. 89A, 89B, and 89C, CMT is 395 and vision is 20/150. It is clear, in this case, to the doctor that statistically the best route would be to follow the recommendation. Not doing so, it is shown the statistical probability even with optimal timing of a 3× improvement in vision with Lucentis versus Eylea 20/50 compared to 20/150.

In the embodiment of FIGS. 87A, 887B, 87C, 87D, and 87E 49 depicts a suggestion that Eylea be used in two weeks from 4/6/2019. This could be due to the fact that in FIGS. 87A, 887B, 87C, 87D, and 87E, 46 shows that Eylea was used on March 21st. In the example of FIGS. 87A, 887B, 87C, 87D, and 87E, the doctor confirms the suggestions and displayed is the statistical likelihood of clinical and diagnostic testing changes using the suggested method of treatment to continue Eylea injections. As such, in FIGS. 87A, 887B, 87C, 87D, and 87E, 52 depicts that, Eylea, if used 29 days later, the CMT is predicted to be 305. In FIGS. 87A, 887B, 87C, 87D, and 87E, on today's visit, 4/6/2019 the CMT is 295, at 46.5, which is a dramatic improvement in the right eye, which can be partly due to the fact that there is only 15 days from the last time 3/21/19, an OCT and Eylea injection were performed, and 15 days perhaps happened to be the maximal effect of Eylea on CMT. With that improvement of 295, depicted at 46.5, the recommendation is made to continue to use Eylea and that is what is being displayed as being done on 4/21/2019 and each month until 57, 9/21/2019, when the injection is performed 60 days later.

In FIGS. 87A, 887B, 87C, 87D, and 87E, 53 displays to the doctor that the CMT is predicted to be 255 on 7/21/2019, which in fact is back to normal which can be highlighted as 255 at 3 in the summary row. In the embodiment of FIGS. 87A, 887B, 87C, 87D, and 87E, that was the patient's best vision and base line of 255, so the predictive scenario displayed can start on 7/21/19 instead of every 30 days to every 60 days. In FIGS. 87A, 887B, 87C, 87D, and 87E, 54 shows the CMT remains at 255 microns even with the injections spread out and it is highlighted because that is the best CMT even with injections now at 60 days instead of 30 days. 58 of FIGS. 87A, 887B, 87C, 87D, and 87E depicts that the injection being spread out to every 90 days, and the predicted values on 5/21/2020, which is 90 days later, are displayed. 55 shows a CMT of 255 and the VA for the first time is 20/25 in 56, which corresponds to 4, the best vision the patient ever had, and, in some embodiments, all can be highlighted to emphasize to the doctor the favorable outcome of following a suggested treatment. Row 72 of FIGS. 87A, 887B, 87C, 87D, and 87E depicts that the result of the suggested treatment is particularly good considering that by 5/21/2020 the patient is now only having injections of Eylea every 90 days and the patients CMT is normal and vision has returned to the best the patient has ever had.

One aspect of the whole life feature is its use as a health planning tool. With this tool, the physicians can visualize the past health status including but not limited to history of physiologic results, medications, procedures, and also visualize potential outcomes for the future under a chosen set of circumstances such as but not limited to effect of different medications.

In one aspect of the whole life feature, data representing potential future health outcomes is generated. This can be done in multiple ways including but not limited to statistical based techniques, machine learning and artificial intelligence based techniques. In one implementation of data generation representing the future health outcomes, the DHR system may initially create a knowledge database. The knowledge database maybe created with expert knowledge or by gathering statistical data or both. As an example, expert opinions about which medication may work best for a patient with a certain type of disease within a certain type of demographic and with certain types of characteristics such as but not limited to age, sex, weight, height etc. may be collected and encoded. Expert opinions may also include more sophistication for example, it may include what may happen if the patient is a smoker and continued to smoke. The knowledge database may also be created using statistical based techniques. In the statistical based technique, patients with a similar disease may be classified using categories suggested by experts. Machine learning and AI technique may also be used to classify such patients. Over time as more data is collected, the effect of different medications or procedures or interventions may become evident for patients that are classified similarly. In a simple non-limiting example, patients with glaucoma may be classified by age, weight, sex, family history and occupation. The effect of different types of medications may be collected as the patient continues to visit the doctor. Overtime, when a statistically significant amount of data has been collected the knowledge base may contain the underlying data representing potential future health outcomes for a given set of patient characteristics. Subsequently, the database may be queried with required inputs to determine such outcomes.

In another aspect of the whole life feature, the future health outcome data is displayed. FIG. 89 shows one example of how the future health outcome data may be displayed. In this example display configuration, the entire display is subdivided into several sections where different types of data are displayed. For example, section 6 shows the actual (past) data including physiologic parameters, medications prescribed and dates of service. Section 8 is a representation of the future as further explained below. Section 10 and 11 illustrate the different medications. Each medication may be color code or tagged uniquely as explained previously. Section 1 illustrates the dates. Section 3 and 4 illustrates two different physiologic parameters spanning the past, present and future. The number of physiologic parameters, the type of parameters to be displayed and analyzed, may be chosen by the physician once the whole life display is invoked. Once the physiologic parameters to be analyzed and displayed are chosen, the data in section 8 (or the future health outcome data) is generated as described previously. Specifically, the DHR tool can create one or multiple queries based on information about the patient and information about how the patients are classified in the knowledgebase. As an example, the knowledge base may contain the underlying data of how a particular medication typically performs for male patients between the ages 50 and 60 with BMI of above 25 for a particular disease. In this case, the query would be constructed by including the age of the patient, the BMI, the disease and the suggested medication. Thus, if the patient has the characteristics of having that specific disease and is between 50 and 60 years of age and has a BMI of over 25, the knowledge database will provide a result. If the disease is glaucoma, an example result may be that the central macular thickness (CMT) decreases over a 12 month time frame with periodic injections of one type of medication. Another query may be formed for the same patient with the same conditions but with a different medication. Here a result may be that according to the data in the knowledge base, the second medication may not result in decreased CMT—it may in fact increase. Such results may be displayed on the display of FIG. 89.

In the example display illustrated in FIG. 89, the physiologic parameters are shown as a line graph (see element 18). In this example, line graph 18 illustrates the CMT values as measured in the previous visits in section 6 (“Actual values”). The physician has chosen to display the effect of medications Eyelea and Lucentis with the patient currently on Eyelea. Section 10 indicates when Eyelea was administered. On the current visit (“Today's visit”), at least two queries were performed; one to determine the effect of Eyelea for patients with similar characteristics and one to determine the effect of Lucentis also with similar characteristics. The underlying data in the knowledge base may show that patients with the particular set of characteristics do not respond well with Eyelea but do respond well with Lucentis. The underlying data may also have a quantified value of how much the CMT is expected to increase or decrease over a period of time. If the increase or decrease is known quantitatively, then the line graph 18 may be extended from today's CMT value to a future value by adding or subtracting from the current value. The future health outcomes may also be displayed as a qualitative result. In other words, in section 8, the DHR can display a message. An example message may be “According to statistical data, this patient is expected to have a decrease of 3% over 1 year with Eyelea”.

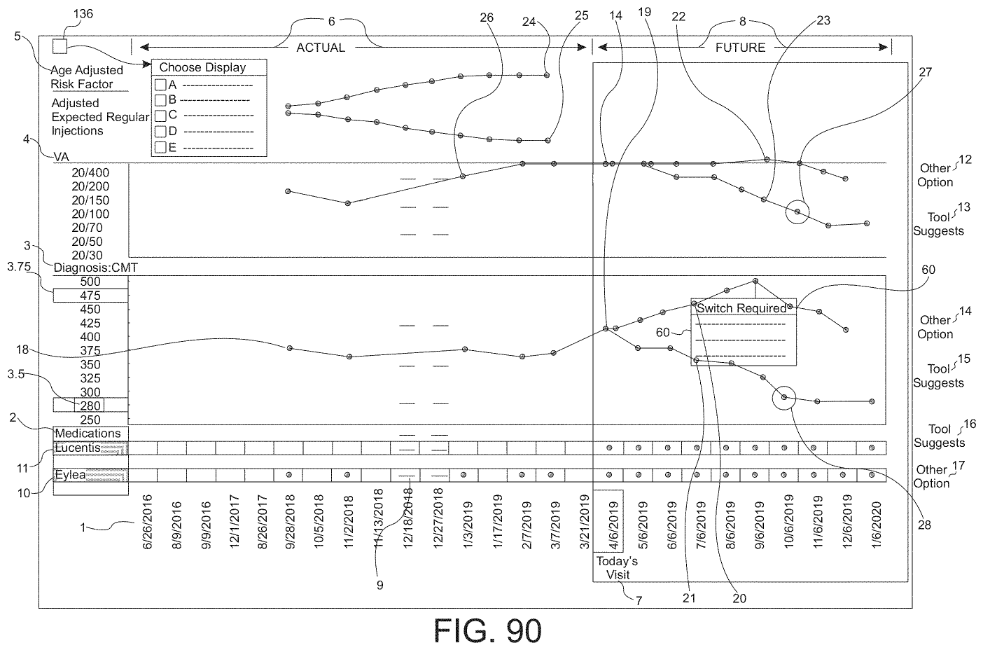

In another concept, representative images are displayed alongside the line graph in either the “Actual” section (Section 6) or the “Future” section (Section 8) as described below. FIG. 90 illustrates the concept. In this example, representations of the OCT images of the eye of the patient is displayed alongside the CMT values in the “Actual” section. The representations may be selected by the physician and the actual image associated with a data point may be displayed on another part of the display such as in the top of the display as illustrated by 37 and 38. The association of the data point to the image may have been done previously by a physician while examining the images and providing information to the patients. Alternatively, automated image analysis software that may include analysis packages based on various techniques such as but not limited to machine learning and AI may be used to choose the representative images.

In another concept, representative images for the “Future” section may also be displayed as described below. Here, of course no actual images of the patient exist because the date has not occurred yet. However, the knowledge database may include images from other patients who share the similar characteristics (or are similarly classified) with the specific patient being examined. Thus, in a prior step before the specific patient is being examined, images from other patients may be collected, classified using one or multiple categories and stored in the knowledge database. As the physician opens the record for a specific patient and activates the whole life feature, queries may be formed by the DHR system and sent to the knowledge database. These queries may then result in images from another one or multiple patients, representation of which may be displayed alongside the results of other queries. As an example, earlier the prediction of CMT was described as it related to which medication was prescribed. In the same manner two sets of images may be displayed from two different patients, both of whom have the same characteristics (or classified similarly) as the specific patient. One set of images may be of a patient on one medication (example Eyelea) and another set of images may be of a patient on another medication (example Lucentis). These types of displays may be useful for various purposes including for the physician to rapidly make decisions about future orders including but not limited to medications or for the physician to educate the patient about his or her condition. In addition, as long as the underlying knowledge database supports it, the physician can investigate “what if” scenarios and visualize the results for analysis or communication.

FIGS. 89A, 89B, and 89C illustrate predictive analytics in accordance with an embodiment of the present principles. In FIGS. 89A, 89B, and 89C, the precited analytics demonstrates to the doctor that the predicted outcome if the doctor chooses to save money and would treat the right eye on 4/21/2019 with Avastin instead of Eylea. In the embodiment of FIGS. 89A, 89B, and 89C CMT increases on 5/21/2019 to 350. By 6/21/2019, the CMT is now highlighted in color (e.g., yellow) because a threshold of worsening is predicted to be reached to an increase in CMT to 360 and the VA worsens to 20/70. In FIGS. 89A, 89B, and 89C, in 3, the worst CMT was 350 and 360 is now augmented. The vision in 5 shows the worst it has been was 20/70 and now, with Avastin on Jun. 21, 2019, the vision has fallen to that poor level 20/70. When the patient comes in at the next visit on 7/21/2019, the prediction highlights the CMT for example in red because it is now significantly worse than even the worst it has ever been at 370 compared to 3 at 350 and the vision is 20/100 on 7/21/2019. Again, significantly worse than in 5. In FIGS. 89A, 89B, and 89C, on 7/21/19, with the vision the lowest ever and CMT also the worst, a mandatory switch to Eylea is suggested, because it has been determined from the data that continuing with Avastin would not be as effective. Therefore, in FIGS. 89A, 89B, and 89C, displayed are the predicted results if Eylea is again started on Jul. 21, 2019, and for the next four visits Eylea is repeated. As depicted in FIGS. 89A, 89B, and 89C, by 11/11/2020 68, the patient's CMT is 280 at 69, which is returning to a more acceptable level, and the vision is predicted to substantially improve to 20/30, but still not the best it has been.

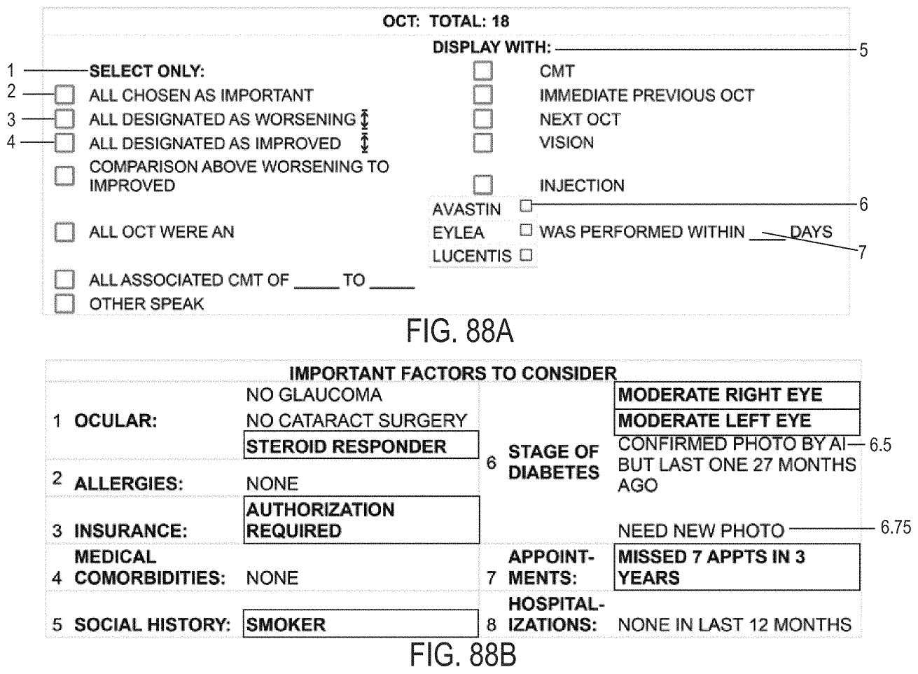

FIG. 90 depicts a multiple panel display that can come up simultaneously with the displays in FIGS. 86, 87, and 88 in accordance with an embodiment of the present principles. In some embodiments, generating a view of FIGS. 90, 91, and 92 can include activating 118 in each of FIGS. 86, 87, and 89. The reverse is also true of FIGS. 90, 91, and 92 (described further below) by activating 136 either the display of FIG. 86, 87, or 89 can be generated or any other configured display can appear. Any number of panels displaying different data and correlating data can be generated on FIG. 90, 91, or 92 and in this whole life view, the panels can be moved around and data can be zoomed into. However, utilizing 136 in FIG. 90-92 enables a user to select what additional data to display.

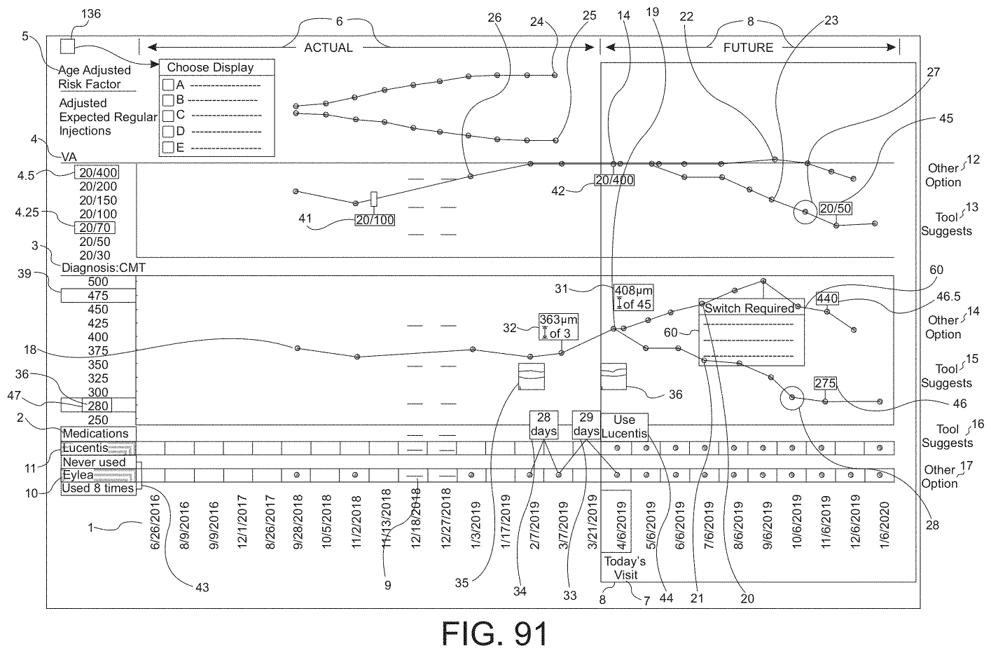

In FIG. 90, 8 displays proposed dates of service in the future, 2 is a medication or a procedure panel, and 10 and 11 display two different color-coded medications, which shows how the patient responds to the treatment and can be compared over time. The effect of these different treatments can be measured in 3 of FIG. 90 where any diagnostic test or procedure can be followed and mapped out depending on what are the results. In FIG. 90, displayed is central macular thickness measured from 250 microns to 500 microns. In some embodiments and as depicted in FIG. 91, 280 can be highlighted in a first color (e.g., green) at 3.5 and 475 microns can be highlighted in a second color (e.g., red) at 3.75 can be highlighted as the best and worst CMT similar to the summary row described in 3 and 7 on FIGS. 87A, 887B, 87C, 87D, and 87E and 89.

In FIG. 90,4 depicts any clinical finding or symptom, illustratively a displayed vision, which is mapped from 20/30 to 20/400 and 5 can depict an age adjusted risk factor or adjusted expected results. In the embodiment of FIG. 90, patients can be shown how they compare to patients with similar demographics, conditions, and treatments. Visually, the doctor can see this as well as the patient, and it can help guide treatment. 24 of FIG. 90 shows how the patient is responding compared to patients shown in 25 with similar conditions and is age adjusted patients with similar issues, and how they would have responded under similar parameters. In FIG. 90, 9 depicts an encounter, with cancelled or missed appointments. The user can now take into consideration if there is a change in the regular plan or resulting measurement that may occur because of a missed appointment. The impact of the missed appointment can be directly visualized.

In FIGS. 90, 13, 15, and 16 are examples of suggestions made by embodiments of the present principles. FIG. 90 correlates to the findings seen in FIGS. 87A, 887B, 87C, 87D, and 87E for the left eye compared to FIGS. 89A, 89B, and 89C in the left eye. 18 of FIG. 90 shows the patient, who was injected with Eylea 10 on Sep. 25, 2018, and the CMT is 373 microns. This can be followed as Eylea injections occur, the CMT is mapped out. At 19 the CMT is shown to be 400 microns and is highlighted in color (e.g., red). This can be set in many ways, but 400 microns is a worsening that can be programmed to create an alert and be highlighted when data represented by a number exceeds a certain threshold. It happens to be correlated by the summary row in FIGS. 89A, 89B, and 89C, at 7. As depicted in FIG. 90, the patient's vision is becoming the worst it has ever been. It can present a dramatic change between Mar. 7, 2019 to now, 408 microns. The CMT with 19. In some embodiments, this is where the decision tree can occur. Does the doctor follow 15, the application suggestion, or another option, 14? Note, a threshold line can be placed on any panel such as 3. If there is a number that a user or the tool feels an action is required if data crosses that threshold, then the user can see if the data is above or below that threshold line. In some embodiments, the user can drag the threshold line and set for an individual patient when actions should be taken.

In FIG. 90, at 21 an improving CMT using Lucentis is displayed in a first color (e.g., orange). 28 shows a continued improvement. In fact, it is highlighted in a second color (e.g., yellow) demonstrating that this patient has now returned to base-line. The CMT is the best it has ever been. In FIG. 90, at 27 it is depicted that the vision has also returned on Oct. 6, 2019 to the best that patient's vision has ever been. So, within each graph there can be great meaning. For instance, additional information about the vision can be obtained on 18. By utilizing 18 anywhere on any visit, the image or clinical data of any kind can appear, and information can be displayed showing the central macular thickness or clinical findings were derived or the actual diagnostic test itself can come up as seen on 35 and 36 of FIG. 91.

FIG. 91 depicts several elements suddenly popping up on the screen highlighted by any method so the doctor can quickly understand the reasoning 31 shows that 19 was an increased thickening with a red arrow of 45 μm from the previous with it being 408 μm on Apr. 6, 2019. simultaneously 32 shows the visit before was 363 μm and was a worsening with a red arrow of 3 microns from the previous OCT. That previous OCT is the one that chosen to display in a thumbnail on 35 and a thumbnail of the OCT which explain the changes seen on 19 and displayed with a thumbnail image of the OCT 36 showing the changes. These thumbnail images 35 and 36 are specifically chosen as the most important images for the doctor to consider. Each OCT image may have 19 slices but the one that's most important to compare is shown.

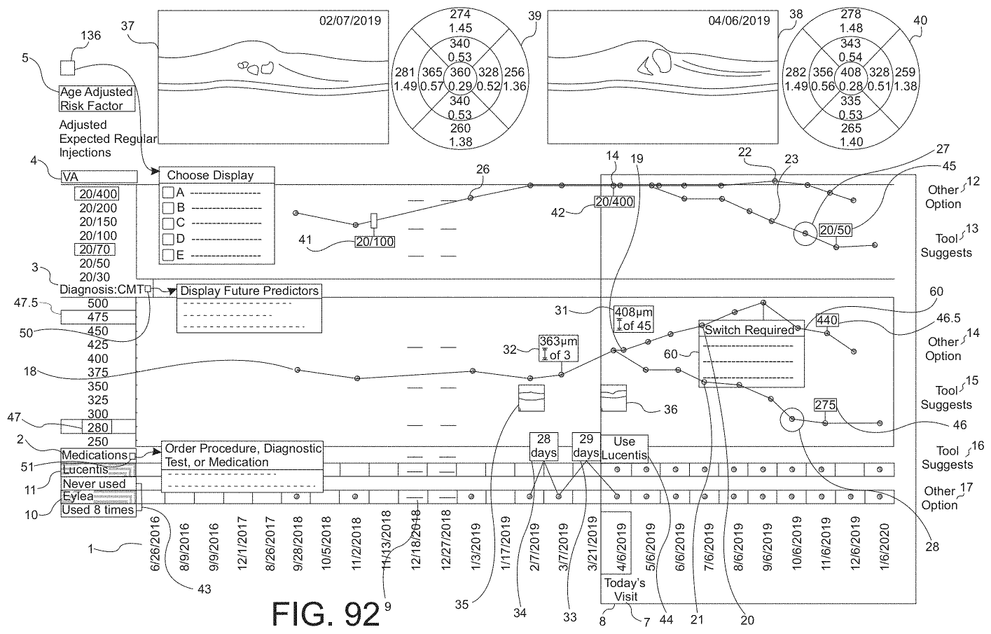

FIG. 92 shows the top panel number five collapsing to make room for the two images 35 and 36, so the doctor can see the enlargement of 35 in 37 shows Feb. 7, 2019° C.T 39 shows multiple measurements with the central measurement being the 360 μm that's most important. 39 shows the enlarged image from Apr. 6, 2019 that was a thumbnail from 36 and again 40 shows the center of all of the different numbers being 408 μm which matches the 31. Now, the tool is able to show the exact images all in context with all other information so doctors can make a decision. Highlighted in 43 is the fact that Eylea was used eight times in the past but Lucentis was never used. This supports the conclusion in 44 where the tool is recommending using Lucentis. Now the tool goes one step further and displays the past compared to the future. 41 shows the vision is 20/100 on Nov. 2, 2018 and it shows an improvement at 20/50 in 45 if the switch is accepted by the doctor to Lucentis. The tool displays 46 and 46.5 comparing the result of predicted results using the different medicines 10 or 11 shown on the line graph of option (Eylea) 10 results in a CMT of 440 μm, 46.5 compared to 46 when the tools suggestion of Lucentis performed and improvement to 275 μm occurs. However, should the user choose not to follow the clinical decision support of the tool in 44 and chooses instead to continue to use Eylea, then when a threshold is reached is alerted and messages can be displayed as seen in 60 in FIG. 90-92. Here the tool has been programmed as described in FIG. 65 to make the recommendation seen in 60, which states switch from current treatment mandated. 22 can display the clinical data why the invention has suggested this CDS recommendation. In one embodiment, the tool can display a predictive image in 38 that shows the tremendous worsening. The tool connects to or in one embodiment has a library of images of the patient's previous images or an archive of images that could represent what the system predicts what the image would look like if a different action is not taken.

In FIGS. 92, 50 and 51 enable a mechanism fora way to bring up onto the single view, a panel that centrals a way to display future predictions for procedures, medications or treatments of any kind as described and shown in 50 of FIGS. 87A, 887B, 87C, 87D, and 87E, and the result through predictive analytics, AI or other means is displayed in 8 of FIG. 91-92. An ordering panel to place orders and confirm orders as described in 90 of FIGS. 87A, 887B, 87C, 87D, and 87E can be similarly on the screen and an example of such a method is by utilizing 50 or 51 of FIG. 92. An ordering panel such as shown on 90, FIGS. 87A, 887B, 87C, 87D, and 87E can also come up on FIG. 82 display so while displaying everything that is relevant, doctors can now confirm their orders seeing the past as well as today's visit and the projected future. These results between two different medicines can also be displayed to the patient to enhance understanding.

The methods and processes described herein may be implemented in software, hardware, or a combination thereof, in different embodiments. In addition, the order of methods can be changed, and various elements can be added, reordered, combined, omitted or otherwise modified. All examples described herein are presented in a non-limiting manner. Various modifications and changes can be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances can be provided for components described herein as a single instance. Boundaries between various components, operations and data stores are somewhat arbitrary, and particular operations are illustrated in the context of specific illustrative configurations. Other allocations of functionality are envisioned and can fall within the scope of claims that follow. Structures and functionality presented as discrete components in the example configurations can be implemented as a combined structure or component. These and other variations, modifications, additions, and improvements can fall within the scope of embodiments as defined in the claims that follow.

In the foregoing description, numerous specific details, examples, and scenarios are set forth in order to provide a more thorough understanding of the present disclosure. It will be appreciated, however, that embodiments of the disclosure can be practiced without such specific details. Further, such examples and scenarios are provided for illustration, and are not intended to limit the disclosure in any way. Those of ordinary skill in the art, with the included descriptions, should be able to implement appropriate functionality without undue experimentation.

References in the specification to “an embodiment,” etc., indicate that the embodiment described can include a particular feature, structure, or characteristic, but every embodiment may not necessarily include the particular feature, structure, or characteristic. Such phrases are not necessarily referring to the same embodiment. Further, when a particular feature, structure, or characteristic is described in connection with an embodiment, it is believed to be within the knowledge of one skilled in the art to affect such feature, structure, or characteristic in connection with other embodiments whether or not explicitly indicated.

Embodiments in accordance with the disclosure can be implemented in hardware, firmware, software, or any combination thereof. Embodiments can also be implemented as instructions stored using one or more machine-readable media, which may be read and executed by one or more processors. A machine-readable medium can include any mechanism for storing or transmitting information in a form readable by a machine (e.g., a computing device or a “virtual machine” running on one or more computing devices). For example, a machine-readable medium can include any suitable form of volatile or non-volatile memory.

Modules, data structures, and the like defined herein are defined as such for ease of discussion and are not intended to imply that any specific implementation details are required. For example, any of the described modules and/or data structures can be combined or divided into sub-modules, sub-processes or other units of computer code or data as can be required by a particular design or implementation.

In the drawings, specific arrangements or orderings of schematic elements can be shown for ease of description. However, the specific ordering or arrangement of such elements is not meant to imply that a particular order or sequence of processing, or separation of processes, is required in all embodiments. In general, schematic elements used to represent instruction blocks or modules can be implemented using any suitable form of machine-readable instruction, and each such instruction can be implemented using any suitable programming language, library, application-programming interface (API), and/or other software development tools or frameworks. Similarly, schematic elements used to represent data or information can be implemented using any suitable electronic arrangement or data structure. Further, some connections, relationships or associations between elements can be simplified or not shown in the drawings so as not to obscure the disclosure.

### EXAMPLES

### Order Examples

1. A computer implemented method of creating medical orders, the method comprising:

generating a dashboard display comprising one or multiple visible panels having data corresponding to different respective medical services;

receiving a request to create an order in response to user interaction with a first one of the multiple panels;

retrieving first medical information as a function of information associated with the panel from which the request was received; and populating a place order panel with the retrieved first medical information.
2. The method of example 1 and further comprising:

receiving order information via the place order panel;

retrieving second medical information in response to the order information; and

populating selected multiple panels with the retrieved second medical information.
3. The method of example 1 wherein the first and second medical information comprise multiple of clinical data, examination data, procedures performed, diagnostic tests and billing information provided in respective ones of the multiple visible panels.
4. The method of example 1 wherein the first one of the multiple panels comprise a procedure panel corresponding to a first procedure, and wherein the second medical information comprises:

diagnostic information correlated to the first procedure for display in a diagnostic panel; and

prior procedures performed correlated to the first procedure for display in the procedure panel.
5. The method of example 4 wherein the second medical information comprises examination data correlated to the first procedure for display in a clinical information panel.
6. The method of example 4 wherein the second medical information comprises medication data correlated to the first procedure for display in a medication information panel.
7. The method of example land further comprising highlighting the second medical information that is displayed in the respective panels.
8. The method of example 1 wherein the first medical information comprises data covering a selected period of time.
9. The method of example 8 wherein the selected period of time comprises past, present and future times.
10. The method of example 1 and further comprising:

receiving user input corresponding to a diagnostic test icon in a diagnostic panel;

retrieving an image corresponding to the diagnostic test; and

displaying the image in an image viewer panel.
12. The method of example 1 wherein the first one of the multiple panels comprises a medication panel, and wherein selection of an order icon from the medication panel generates an overlay for ordering medications.
13. The method of example 1 wherein one of the multiple panels comprises a clinical decision support panel that include clinical decision support information that includes supporting data.
14. The method of example 1 wherein one of the multiple panels comprises a diagnostic panel populated with retrieved first medical information comprising historical diagnoses data.
15. The method of example 1 wherein the first one of the multiple panels comprises a list of patients, and wherein the request to create an order is associated with a selected one of the patients.
16. A machine-readable storage device having instructions for execution by a processor of a machine to cause the processor to perform operations to perform a method, the operations comprising:

generating a dashboard display comprising one or multiple visible panels having data corresponding to different respective medical services;

receiving a request to create an order in response to user interaction with a first one of the multiple panels;

retrieving first medical information as a function of information associated with the panel from which the request was received; and

populating a place order panel with the retrieved first medical information.
17. The device of example 16, the operations further comprising:

receiving order information via the place order panel;

retrieving second medical information in response to the order information; and

populating selected multiple panels with the retrieved second medical information.
18. The device of example 16 wherein the first and second medical information comprise multiple of clinical data, examination data, procedures performed, diagnostic tests and billing information provided in respective ones of the multiple visible panels.
19. The device of example 16 wherein the first one of the multiple panels comprise a procedure panel corresponding to a first procedure, and wherein the second medical information comprises:

diagnostic information correlated to the first procedure for display in a diagnostic panel; and

prior procedures performed correlated to the first procedure for display in the procedure panel.
20. A device comprising:

a processor; and

a memory device coupled to the processor and having a program stored thereon for execution by the processor to perform operations comprising:
generating a dashboard display comprising one or multiple visible panels having data corresponding to different respective medical services;
receiving a request to create an order in response to user interaction with a first one of the multiple panels;
retrieving first medical information as a function of information associated with the panel from which the request was received; and
populating a place order panel with the retrieved first medical information.
21. The device of example 20, the operations further comprising:

receiving order information via the place order panel;

retrieving second medical information in response to the order information; and

populating selected multiple panels with the retrieved second medical information.

### Medication Display Examples

1. A computer implemented method of displaying patient medications over time, the method comprising:

accessing event data representative of patient events, the event data including an event date and a first medication identifier for each patient event;

generating a display including a list of patient events in chronological order in a first axis of the display;

associating the event dates and medication identifier to identify a time period corresponding to the medication; and

representing the time period in the display in a second axis transverse to the first axis and having an attribute spanning the time period corresponding to the patient events that include the first medication.
2. The method of example 1 wherein the attribute is indicative of the type of drug.
3. The method of example 2 wherein the attribute is a color assignable by a user.
4. The method of example 1 wherein the event data includes description of treatments selected from at least one of a procedure and a measured parameter.
5. The method of example 1 wherein the first axis includes a row for each patient event and the second axis includes a column.
6. The method of example and wherein the patient events include a second medication having an attribute different than the first medication.
7. A method for medication management and display in a data command center comprising one or more windows for display and including information from at least one database, the data command center displaying on a screen, using the one or more windows, at least one of medical services, clinical data, examination findings, diagnostic tests, and procedures performed on one or more patients, the one or more windows comprising a plurality of data fields for displaying the information received or derived from the at least one database, wherein the at least one of the medical services, the clinical data, the examination findings, the diagnostic tests, and the procedures are arranged in on the screen according to at least one of a time and a date that the medical services, the clinical data, the examination findings, the diagnostic tests and the procedures were performed on the one or more patients, the method comprising:

determining, from the at least one database and the at least one of the medical services, the clinical data, the examination findings, the diagnostic tests, and the procedures, medications administered to the one or more patients;

generating a respective graphical representation for each of the determined medications administered to the one or more patients; and

displaying at least one generated, respective graphical representation of at least one medication administered to a patient in the at least one or more windows in context with at least the information from the at least one patient database and the at least one of the medical services, the clinical data, the examination findings, the diagnostic tests, and the procedures,

wherein the at least one generated, respective graphical representation of the at least one medication administered to the patient is arranged in on the screen according to at least one of the times and the dates that the at least one medication was being administered to the patient;

wherein the at least one or more windows displaying the graphical representations are dynamically collapsible and expandable.
8. The method of example 7, wherein the at least one generated, respective graphical representation enables a user to immediately identify on sight a respective medication without having to read a name of the medication.
9. The method of example 7, wherein the generated, respective graphical representations represent at least one of individual medications, classes of medications, combinations of medications, or logical groupings of medications.
10. The method of example 7, wherein the generated, respective graphical representations differentiate medications by at least one of color, combinations of colors, or symbols.
11. The method of example 10, wherein the colors are colors standardized by the American Academy of Ophthalmology.
12. The method of example 7, wherein respective graphical representations are generated for and separately listed by each condition of a patient for which medications are being administered.
13. The method of example 7, further comprising:

generating and displaying an alert if a medication associated with a respective one of the one or more patients has changed since a last visit.
14. A data command center visual display system that displays data on a display screen, comprising:

a computing device comprising at least one processor;

a non-transitory computer-readable medium, having stored thereon, software instructions that when executed by the at least one processor of the computing device, cause the computing device to perform operations comprising at least:

linking to and receiving patient related medical records including patient data from at least one patient data source, wherein the patient data includes at least one of medical services, clinical data, examination findings, diagnostic tests, and procedures performed on one or more patients;

determining, from at least one of the patient data and the at least one of the medical services, the clinical data, the examination findings, the diagnostic tests, and the procedures, medications administered to each of the one or more patients;

generating a respective graphical representation for each of the determined medications administered to each of the one or more patients; and

displaying using the one or more windows, at least one of medical services, clinical data, examination findings, diagnostic tests, and procedures performed on one or more patients and at least one generated, respective graphical representation of at least one medication administered to a patient in context with at least one of the patient data and the at least one of the medical services, the clinical data, the examination findings, the diagnostic tests, and the procedures;

wherein the at least one of the medical services, the clinical data, the examination findings, the diagnostic tests, and the procedures are arranged on the screen according to at least one of a time and a date that the medical services, the clinical data, the examination findings, the diagnostic tests and the procedures were performed on the one or more patients;

wherein the at least one generated, respective graphical representation of the at least one medication administered to the patient is arranged on the screen according to at least one of the times and the dates that the at least one medication was being administered to the patient; and

wherein the at least one or more windows displaying the graphical representations are dynamically collapsible and expandable.
15. The data command center visual data system of example 14, wherein the at least one generated, respective graphical representation enables a user to immediately identify on sight a respective medication without having to read a name of the medication.
16. The data command center visual display system of example 14, wherein the generated, respective graphical representations represent at least one of individual medications, classes of medications, combinations of medications, or logical groupings of medications.
17. The data command center visual display system of example 14, wherein the generated, respective graphical representations differentiate medications by at least one of color, combinations of colors, or symbols.
18. The data command center visual display system of example 17, wherein the colors are colors standardized by the American Academy of Ophthalmology.
19. The data command center visual display system of example 14, wherein respective graphical representations are generated for and separately listed by each condition of a patient for which medications are being administered.
20. The data command center visual display system of example 14, wherein the computing device is further configured to:
generate and display an alert if a medication being administered to the one or more patients has changed since a last visit.

### Dynamic Data Examples

1. A method for dynamic data display in a data command center comprising a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method comprising:

receiving patient-related data from the at least one patient database;

displaying patient-related data in predetermined, respective ones of the data entry fields;

in response to an entry or change of patent-related data in at least one of the respective ones of the data entry fields, making a change to the data in at least one other of the respective ones of the data entry fields.
2. The method of example 1, wherein the change to the data in the at least one other of the respective ones of the data entry fields includes at least one of expanding the data representation, truncating the data representation, adding an alert to the data representation, highlighting the data representation, adding a representation to the data entry field indicating that data is missing, adding a graphical representation indicating access to underlying information, adding a thumbnail representation, adding a text link data representation, or including a Summary Data Representation, which illustrates a single data representation comprised of multiple data sources.
3. The method of example 1, wherein data entry fields are expanded or truncated depending on user specialty or interests.
4. A data command center visual display system that displays data on a display screen, comprising:

a computing device comprising at least one processor;

a non-transitory computer-readable medium, having stored thereon, software instructions that when executed by the at least one processor of the computing device, cause the computing device to perform operations comprising at least:

linking to and receiving patient related medical records including patient data from at least one patient data source; and

displaying a medical records dashboard including one or more windows, the medical record dashboard capable of displaying, using the one or more windows, patient data from at least one patient data source including at least one of medical services, clinical data, examination findings, diagnostic tests, and the procedures performed on one or more patients, the one or more windows comprising a plurality of data entry fields, including at least one collapsible data entry field, for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients;

wherein a display of patient data in the medical records dashboard includes dynamic data fields in which:

in response to an entry or change of patent-related data in at least one of the respective ones of the data entry fields, a change is made to the data in at least one other of the respective ones of the data entry fields.
5. The data command center visual display system of example, wherein the change to the data in the at least one other of the respective ones of the data entry fields includes at least one of expanding the data representation, truncating the data representation, adding an alert to the data representation, highlighting the data representation, adding a representation to the data entry field indicating that data is missing, adding a graphical representation indicating access to underlying information, adding a thumbnail representation, adding a text link data representation, or including a Summary Data Representation, which illustrates a single data representation comprised of multiple data sources.
6. The data command center visual display system of example 14 wherein data entry fields are expanded or truncated depending on user specialty or interests.

### Whole Life Examples

1. A computer implemented method comprising:
determining a timeline for a whole life view of data associated with multiple selected parameters for which medical data corresponding to the patient is available;
accessing medical data, including a patient's medical data corresponding to the multiple selected parameters;
conforming the accessed medical data to the timeline; and displaying each of the selected parameters adjacent to each other along the timeline, wherein the display is zoomable along the timeline.
2. The method of example 1 and further comprising:

predicting future values for the selected parameters; and

displaying predicted future values along the timeline.
3. The method of example 2 wherein the predicted future values are selected based on machine learning rules trained on data from multiple patients.
4. The method of example 1 wherein the selected parameters include data related to procedures, labs, diagnoses, and medications.
5. The method of example 1 wherein the multiple selected parameters are selected as a function of a first parameter selected by a user from one of multiple panels displaying different sets of data.
6. The method of example 1 wherein the timeline is selected based on rules, including a first record in time corresponding to at least one of the selected parameters.
7. The method of example 1 wherein the display is temporally zoomable to alter the timeline to show more or less information.
8. The method of example 1 wherein the display comprises graphical representations of the selected parameters including links to images.
9. The method of example 7 wherein the graphical representations of data include procedure codes that operate as links to procedure details.
10. The method of example 1 wherein the medical data includes non-patient data and the non-patient data comprises life expectancy data of patients having a disease that is similar to a disease of the patient.
11. The method of example 1 wherein the medical data includes non-patient data and the non-patient data comprises data corresponding to how a cohort of other patients react to a selected drug.

This disclosure is to be considered as exemplary and not restrictive in character, and all changes and modifications that come within the guidelines of the disclosure are desired to be protected.

What is claimed is:

1. A computer implemented method of creating medical orders, the method comprising:
generating a dashboard display comprising one or multiple visible panels having data corresponding to different respective medical services;
receiving a request to create an order in response to user interaction with a first one of the multiple panels;
retrieving first medical information as a function of information associated with the panel from which the request was received; and
populating a place order panel with the retrieved first medical information.

2. The method of claim 1 and further comprising:
receiving order information via the place order panel;
retrieving second medical information in response to the order information; and
populating selected multiple panels with the retrieved second medical information.

3. The method of claim 1 wherein the first and second medical information comprise multiple of clinical data, examination data, procedures performed, diagnostic tests and billing information provided in respective ones of the multiple visible panels.

4. The method of claim 1 wherein the first one of the multiple panels comprise a procedure panel corresponding to a first procedure, and wherein the second medical information comprises:
diagnostic information correlated to the first procedure for display in a diagnostic panel; and
prior procedures performed correlated to the first procedure for display in the procedure panel.

5. The method of claim 4 wherein the second medical information comprises examination data correlated to the first procedure for display in a clinical information panel.

6. The method of claim 4 wherein the second medical information comprises medication data correlated to the first procedure for display in a medication information panel.

7. The method of claim 1 and further comprising highlighting the second medical information that is displayed in the respective panels.

8. The method of claim 1 wherein the first medical information comprises data covering a selected period of time.

9. The method of claim 8 wherein the selected period of time comprises past, present and future times.

10. The method of claim 1 and further comprising:
receiving user input corresponding to a diagnostic test icon in a diagnostic panel;
retrieving an image corresponding to the diagnostic test; and
displaying the image in an image viewer panel.

11. The method of claim 1 wherein the first one of the multiple panels comprises a medication panel, and wherein selection of an order icon from the medication panel generates an overlay for ordering medications.

12. The method of claim 1 wherein one of the multiple panels comprises a clinical decision support panel that include clinical decision support information that includes supporting data.

13. The method of claim 1 wherein one of the multiple panels comprises a diagnostic panel populated with retrieved first medical information comprising historical diagnoses data.

14. The method of claim 1 wherein the first one of the multiple panels comprises a list of patients, and wherein the request to create an order is associated with a selected one of the patients.

15. A machine-readable storage device having instructions for execution by a processor of a machine to cause the processor to perform operations to perform a method, the operations comprising:
generating a dashboard display comprising one or multiple visible panels having data corresponding to different respective medical services;
receiving a request to create an order in response to user interaction with a first one of the multiple panels;
retrieving first medical information as a function of information associated with the panel from which the request was received; and
populating a place order panel with the retrieved first medical information.

16. The device of claim 15, the operations further comprising:
receiving order information via the place order panel;
retrieving second medical information in response to the order information; and
populating selected multiple panels with the retrieved second medical information.

17. The device of claim 15 wherein the first and second medical information comprise multiple of clinical data, examination data, procedures performed, diagnostic tests and billing information provided in respective ones of the multiple visible panels.

18. The device of claim 15 wherein the first one of the multiple panels comprise a procedure panel corresponding to a first procedure, and wherein the second medical information comprises:
diagnostic information correlated to the first procedure for display in a diagnostic panel; and
prior procedures performed correlated to the first procedure for display in the procedure panel.

19. A device comprising:
a processor; and
a memory device coupled to the processor and having a program stored thereon for execution by the processor to perform operations comprising:
generating a dashboard display comprising one or multiple visible panels having data corresponding to different respective medical services;
receiving a request to create an order in response to user interaction with a first one of the multiple panels;
retrieving first medical information as a function of information associated with the panel from which the request was received; and
populating a place order panel with the retrieved first medical information.

20. The device of claim 19, the operations further comprising:
receiving order information via the place order panel;
retrieving second medical information in response to the order information; and
populating selected multiple panels with the retrieved second medical information.
